(19) United States
(12) Patent Application Publication
(10) Pub. No.: US 2014/0317781 A1
(43) Pub. Date: Oct. 23, 2014

(54) ISOLATED POLYNUCLEOTIDES AND POLYPEPTIDES, TRANSGENIC PLANTS COMPRISING SAME AND USES THEREOF IN IMPROVING ABIOTIC STRESS TOLERANCE, NITROGEN USE EFFICIENCY, BIOMASS, VIGOR OR YIELD OF PLANTS

(71) Applicant: A.B. Seeds Ltd., Lod (IL)

(72) Inventors: Rudy Maor, Rechovot (IL); Iris Nesher, Tel-Aviv (IL); Orly Noivirt-Brik, Givataim (IL); Osnat Yanai-Azulay, Rishon-LeZion (IL)

(21) Appl. No.: 14/354,932

(22) PCT Filed: Oct. 31, 2012
(86) PCT No.: PCT/IL2012/050431

§ 371 Date: Apr. 29, 2014

Related U.S. Application Data
(60) Provisional application No. 61/553,340, filed on Oct. 31, 2011.
(60) Provisional application No. 61/553,355, filed on Oct. 31, 2011.

(51) Int. Cl. C12N 15/82

(52) U.S. Cl. CPC C12N 15/8273; C12N 15/8261; C12N 15/8271
USPC 800/286; 800/278; 800/290; 800/298; 536/23.1; 435/320.1; 800/289

(57) ABSTRACT

Methods of improving abiotic stress tolerance, nitrogen use efficiency, biomass, vigor or yield of a plant are provided. According to an aspect the method comprising expressing within the plant an exogenous polynucleotide having a nucleic acid sequence at least 90% identical to SEQ ID NOs: 103, 101-102, 104-216, 223-227, 264-416, wherein the nucleic acid sequence is capable of regulating abiotic stress tolerance of the plant, thereby improving abiotic stress tolerance, nitrogen use efficiency, biomass, vigor or yield of the plant. Alternatively, the method comprises, expressing within the plant an exogenous polynucleotide which downregulates an activity or expression of a gene encoding an RNAi molecule having a nucleic acid sequence at least 90% identical to a nucleic acid sequence selected from the group consisting of SEQ ID NOs: 1-100, 615-626 and 639, thereby improving abiotic stress tolerance, nitrogen use efficiency, biomass, vigor or yield of a plant.

### FIELD AND BACKGROUND OF THE INVENTION

The present invention, in some embodiments thereof, relates to isolated polynucleotides and polypeptides, transgenic plants comprising same and uses thereof in improving abiotic stress tolerance, nitrogen use efficiency, biomass, vigor or yield of plants

Abiotic stress (ABST) is a collective term for numerous extreme environmental parameters such as drought, high or low salinity, high or low temperature/light, and nutrient imbalances. The major agricultural crops (corn, rice, wheat, canola and soybean) account for over half of total human caloric intake, giving their overall yield and quality vast importance. Abiotic stress causes more than 50% yield loss of the above mentioned major crops. Among the various abiotic stresses, drought is the major factor that limits crop productivity worldwide. Furthermore, drought is associated with increase susceptibility to various diseases. Abiotic-stress-induced dehydration or osmotic stress, in the form of reduced availability of water and disruption of turgor pressure, causes irreversible cellular damage. A water-limiting environment at various plant developmental stages may activate various physiological changes.

Water deficit, salinity and low/high temperatures are stresses that cause plant cellular dehydration, due to transpiration rate that exceeds water uptake. Drought is known to elicit a response in the plant that mainly affects root architecture, causing activation of plant metabolic pathways driven to maximize water assimilation. Improvement of root architecture, i.e. making branched and longer roots, allows the plant to reach water and nutrient/fertilizer deposits located deeper in the soil by an increase in soil coverage. Thus, genes governing enhancement of root architecture may be used to improve drought tolerance.

High salt levels, or salinity, of the soil acts similarly to drought; it prevents roots from extracting water and nutrients and thus reduces the availability of arable land and crop production worldwide, since none of the top five food crops can tolerate excessive salt. Salinity causes a water deficit which leads to osmotic stress (similar to freezing and drought stress) and critically damages biochemical processes. Large land areas throughout the world naturally have high salt levels and thus are currently uncultivable. In regions that rely heavily on agricultural production, soil salinity is a significant problem expected to worsen due to growing population and extreme climatic changes. Since salt accumulates in the upper soil layer where seeds are placed, and may interfere with their germination, salt tolerance is of particular importance early in a plant's lifecycle.

Temperature is a critical factor in germination of many crops. Seedlings as well as mature plants that are exposed to excess heat may experience heat shock, which may arise in various organs when transpiration is insufficient to overcome heat stress. Heat shock damages cellular structures and impairs membrane function and overall protein synthesis (except that of heat shock proteins). Heat stress often accompanies conditions of low water availability, such as drought, and the combined stress can fatally alter plant metabolism. Dehydration invokes survival strategies in plants that include structural (lower surface area) as well as cellular content (increase in oil and soluble material) modifications to prevent evaporation and water loss caused by heat, drought, or salinity.

There is great variability in responses to abiotic stress among different plant species, but differences also exist among varieties and cultivars within the same plant species. Certain plants are inherently more tolerant to abiotic stress than others, making their genotypes an attractive research subject for potential identification of drought associated genes. The response to any stress may involve both stress specific and common stress pathways, and drains energy from the plant, eventually resulting in lowered yield. Thus, distinguishing between the genes activated in each pathway and subsequent manipulation of only specific relevant genes could lead to a partial stress response without the parallel loss in yield.

With a growing world population, increasing demand for food, fuel and fiber, and a changing climate, agriculture faces unprecedented challenges. In general, shortage in water supply is one of the most severe global agricultural problems affecting plant growth and crop yield. To illustrate, large areas of cornfields in the United States may be affected by at least moderate drought in any given year. Excessive efforts are made to alleviate the harmful effects of desertification of the world's arable land. Farmers are seeking advanced, biotechnology-based solutions to enable them to obtain stable high yields and give them the potential to reduce irrigation costs or to grow crops in areas where potable water is a limiting factor. It should be noted that improved ABST will confer plants with improved vigor also under non-stress conditions, resulting in crops having improved biomass and/or yield.

Two major small RNA molecules, microRNAs (miRNAs) or small interfering RNAs (siRNAs), potently regulate specific down-regulation/silencing of a target gene, through RNA interference (RNAi). Both miRNAs and siRNAs are oligonucleotides (20-24 bps) processed from longer RNA precursors by Dicer-like ribonucleases, although the source of their precursors is different (i.e., local single RNA molecules with imperfect stem-loop structures for miRNA, and long, double-stranded precursors potentially from bimolecular duplexes for siRNA). Additional characteristics that differentiate miRNAs from siRNAs are their sequence conservation level between related organisms (high in miRNAs, low to non-existent in siRNAs), regulation of genes unrelated to their locus of origin (typical for miRNAs, infrequent in siRNAs) and the genetic requirements for their respective functions are somewhat dissimilar in many organisms. Despite all their differences, miRNAs and siRNAs are overall chemically and functionally similar and both are incorporated into silencing complexes, wherein they can guide post-transcriptional repression of multiple target genes, and thus function catalytically.

In contrast to the abundance of genes involved in the responses to abiotic stress in plants, there is limited information on small RNA molecules involved in plant response and adaptation to abiotic stress.

### Related Background Art

WO 2011/067745

### SUMMARY OF THE INVENTION

According to an aspect of some embodiments of the present invention there is provided a method of improving abiotic stress tolerance, nitrogen use efficiency, biomass, vigor or yield of a plant, the method comprising expressing within the plant an exogenous polynucleotide having a nucleic acid sequence at least 90% identical to SEQ ID NOs: 103, 101-102, 104-216, 223-227, 264-416, wherein said nucleic acid sequence is capable of regulating abiotic stress tolerance of the plant, thereby improving abiotic stress tolerance, nitrogen use efficiency, biomass, vigor or yield of the plant.

According to an aspect of some embodiments of the present invention there is provided a transgenic plant exogenously expressing a polynucleotide having a nucleic acid sequence at least 90% identical to SEQ ID NOs: 1-216, 223-227, 264-416, 615-626 or 639, wherein said nucleic acid sequence is capable of regulating abiotic stress tolerance of the plant.

According to some embodiments of the invention, said polynucleotide has a nucleic acid sequence selected from the group consisting of SEQ ID NOs: 1-216, 223-227, 264-416, 615-626 or 639.

According to some embodiments of the invention, said exogenous polynucleotide encodes a precursor of said nucleic acid sequence.

According to some embodiments of the invention, said precursor is at least 60% identical to SEQ ID NO: 217-222, 417-421 or 458-614.

According to some embodiments of the invention, said exogenous polynucleotide encodes a miRNA or a precursor thereof.

According to some embodiments of the invention, said exogenous polynucleotide encodes a siRNA.

According to some embodiments of the invention, said exogenous polynucleotide is selected from the group consisting of SEQ ID NO: 103, 101-102, 104-216, 217-222, 223-227, 264-416, 417-421 or 458-614.

According to an aspect of some embodiments of the present invention there is provided an isolated polynucleotide having a nucleic acid sequence at least 90% identical to SEQ ID NO: 16-113, 117-216, wherein said nucleic acid sequence is capable of regulating abiotic stress tolerance of a plant.

According to some embodiments of the invention, said nucleic acid sequence us as set forth in SEQ ID NO: 16-113, 117-216

According to some embodiments of the invention, said polynucleotide encodes a precursor of said nucleic acid sequence.

According to some embodiments of the invention, said polynucleotide encodes a miRNA or a precursor thereof.

According to some embodiments of the invention, said polynucleotide encodes a siRNA.

According to an aspect of some embodiments of the present invention there is provided a method of improving abiotic stress tolerance, nitrogen use efficiency, biomass, vigor or yield of a plant, the method comprising expressing within the plant an exogenous polynucleotide which downregulates an activity or expression of a gene encoding an RNAi molecule having a nucleic acid sequence at least 90% identical to a nucleic acid sequence selected from the group consisting of SEQ ID NOs: 1-100, 615-626 and 639, thereby improving abiotic stress tolerance, nitrogen use efficiency, biomass, vigor or yield of a plant.

According to an aspect of some embodiments of the present invention there is provided a transgenic plant exogenously expressing a polynucleotide which downregulates an activity or expression of a gene encoding an RNAi molecule having a nucleic acid sequence at least 90% identical to a nucleic acid sequence selected from the group consisting of SEQ ID NOs: 1-100, 615-626 and 639.

According to an aspect of some embodiments of the present invention there is provided an isolated polynucleotide which downregulates an activity or expression of a gene encoding an RNAi molecule having a nucleic acid sequence selected from the group consisting of SEQ ID NOs: 1-100, 615-626 and 639, 627-638 and 640.

According to some embodiments of the invention, said polynucleotide encodes a miRNA-Resistant Target as set forth in Tables 14-16.

According to some embodiments of the invention, said polynucleotide encoding miRNA-Resistant Target is as set forth in SEQ ID NO: 877-886, 893-913, 1226-1535.

According to some embodiments of the invention, said isolated polynucleotide encodes a target mimic as set forth in Tables 17-19.

According to some embodiments of the invention, said polynucleotide encoding said target mimic is as set forth in SEQ ID NO:1741-1815.

According to an aspect of some embodiments of the present invention there is provided a method of improving abiotic stress tolerance, nitrogen use efficiency, biomass, vigor or yield of a plant, the method comprising expressing within the plant an exogenous polynucleotide encoding a polypeptide having an amino acid sequence at least 80% homologous to SEQ ID NOs: 1861-1869, 1892-1915, 1921-1924, 1931-1939, 1952-1963, 2010-2014, 2327-2355, 2763-3040, 3044-3163, 3175-3269, 3313-3323, 3458-3944 or 3950-3969, wherein said polypeptide is capable of regulating abiotic stress tolerance of the plant, thereby improving abiotic stress tolerance, nitrogen use efficiency, biomass, vigor or yield of the plant.

According to an aspect of some embodiments of the present invention there is provided a transgenic plant exogenously expressing a polynucleotide encoding a polypeptide having an amino acid sequence at least 80% homologous to SEQ ID NOs: 1816-2014, 2183-2355, 2500-3969, wherein said polypeptide is capable of regulating nitrogen use efficiency of the plant.

According to an aspect of some embodiments of the present invention there is provided a nucleic acid construct comprising a polynucleotide encoding a polypeptide having an amino acid sequence at least 80% homologous to SEQ ID NOs: 1816-2014, 2183-2355, 2500-3969, wherein said polypeptide is capable of regulating abiotic stress tolerance of a plant, and wherein said polynucleotide is under a transcriptional control of a cis-acting regulatory element.

According to some embodiments of the invention, said polynucleotide is selected from the group consisting of SEQ ID NO: 2053-2061, 2080-2101, 2106-2109, 2111-2116, 2126-2136, 2178-2182, 2478-2499, 4185-4418, 4422-4527, 4539-4624, 4661-4670, 4787-5213 and 5219-5238.

According to some embodiments of the invention, said polypeptide is selected from the group consisting of SEQ ID NO: 1861-1869, 1892-1915, 1921-1924, 1931-1939, 1952-1963, 2010-2014, 2327-2355, 2763-3040, 3044-3163, 3175-3269, 3313-3323, 3458-3944 and 3950-3969.

According to an aspect of some embodiments of the present invention there is provided a method of improving abiotic stress tolerance, nitrogen use efficiency, biomass, vigor or yield of a plant, the method comprising expressing within the plant an exogenous polynucleotide which downregulates an activity or expression of a polypeptide having an amino acid sequence at least 80% homologous to SEQ ID NOs: 1816-1860, 1870-1891, 1916-1920, 1925-1930, 1940-1951, 1964-2009, 2183-2326, 2500-2762, 3041-3043, 3164-3174, 3270-3312, 3324-3457, 3945-3949, wherein said polypeptide is capable of regulating abiotic stress tolerance of the plant, thereby improving abiotic stress tolerance, nitrogen use efficiency, biomass, vigor or yield of the plant.

According to an aspect of some embodiments of the present invention there is provided a transgenic plant exogenously expressing a polynucleotide which downregulates an activity or expression of a polypeptide having an amino acid sequence at least 80% homologous to SEQ ID NOs: 1816-1860, 1870-1891, 1916-1920, 1925-1930, 1940-1951, 1964-2009, 2183-2326, 2500-2762, 3041-3043, 3164-3174, 3270-3312, 3324-3457, 3945-3979, wherein said polypeptide is capable of regulating abiotic stress tolerance of the plant.

According to an aspect of some embodiments of the present invention there is provided a nucleic acid construct comprising a polynucleotide which downregulates an activity or expression of a polypeptide having an amino acid sequence at least 80% homologous to SEQ ID NOs: 1816-1860, 1870-1891, 1916-1920, 1925-1930, 1940-1951, 1964-2009, 2183-2326, 2500-2762, 3041-3043, 3164-3174, 3270-3312, 3324-3457, 3945-3949, wherein said polypeptide is capable of regulating abiotic stress tolerance of a plant, said nucleic acid sequence being under the regulation of a cis-acting regulatory element.

According to some embodiments of the invention, said polynucleotide acts by a mechanism selected from the group consisting of sense suppression, antisense suppression, ribozyme inhibition, gene disruption.

According to some embodiments of the invention, said cis-acting regulatory element comprises a promoter.

According to some embodiments of the invention, said promoter comprises a tissue-specific promoter.

According to some embodiments of the invention, said tissue-specific promoter comprises a root specific promoter.

According to some embodiments of the invention, the method further comprises growing the plant under water deprivation conditions.

According to some embodiments of the invention, the method further comprises growing the plant under salinity stress.

According to some embodiments of the invention, the method further comprises growing the plant under high temperature stress.

According to some embodiments of the invention, the method further comprises growing the plant under abiotic stress.

According to some embodiments of the invention, said abiotic stress is selected from the group consisting of salinity, drought, water deprivation, flood, etiolation, low temperature, high temperature, heavy metal toxicity, anaerobiosis, nutrient deficiency, nutrient excess, atmospheric pollution and UV irradiation.

According to some embodiments of the invention, the plant is a dicotyledon.

According to some embodiments of the invention, the plant is a monocotyledon.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a plasmid map of the binary vector pORE-E1, which can be used for plant transformation according to some embodiments of the present invention.

FIG. 2 is a schematic description of miRNA assay including two steps, stem-loop RT and real-time PCR. Stem-loop RT primers bind to at the 3′ portion of miRNA molecules and are reverse transcribed with reverse transcriptase. Then, the RT product is quantified using conventional TaqMan PCR that includes miRNA-specific forward primer and reverse primer. The purpose of tailed forward primer at 5′ is to increase its melting temperature (Tm) depending on the sequence composition of miRNA molecules (Slightly modified from Chen et al. 2005, Nucleic Acids Res 33(20):e179). FIGS. 3A-B are schematic illustrations of an artificial miRNA sequence design for predicted siRNA 55507 (SEQ ID NO: 102) on the backbone of ath-miR172a (SEQ ID NO: 453).

FIGS. 4A-B are schematic illustrations of an artificial miRNA sequence design for predicted siRNA 55937 (SEQ ID NO: 2) on the backbone of ath-miR319a (SEQ ID NO: 455).

### DESCRIPTION OF SPECIFIC EMBODIMENTS OF THE INVENTION

The present invention, in some embodiments thereof, relates to isolated polynucleotides and polypeptides, transgenic plants comprising same and uses thereof in improving abiotic stress tolerance, nitrogen use efficiency, biomass, vigor or yield of plants

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

A number of abnormal environment parameters such as drought, salinity, cold, freezing, high temperature, anoxia, high light intensity and nutrient imbalances etc. are collectively termed as abiotic stresses. Abiotic stresses lead to dehydration or osmotic stress through reduced availability of water for vital cellular functions and maintenance of turgor pressure. Stomata closure, reduced supply of CO₂ and slower rate of biochemical reactions during prolonged periods of dehydration, high light intensity, high and low temperatures lead to high production of Reactive Oxygen Intermediates (ROI) in the chloroplasts causing irreversible cellular damage and photo inhibition.

Understanding the molecular mechanism for providing protection against biotic and abiotic stresses may lead to the identification of genes associated with stress tolerance. Optimum homeostasis is always a key to living organisms for adjusted environments.

While reducing the present invention to practice, the present inventors have uncovered dsRNA sequences that are differentially expressed in maize plants grown under abiotic stress conditions including, salt stress, heat stress and drought, versus maize plants grown under optimal conditions. Following extensive experimentation and screening the present inventors have identified double stranded RNA interfering (RNAi) molecules including siRNA and miRNA sequences that are upregulated or downregulated in roots and leaves, and suggest using same or sequences controlling same in the generation of transgenic plants having improved abiotic stress tolerance.

Each of the above mechanisms may affect water uptake as well as salt absorption and therefore embodiments of the invention further relate to enhancement of abiotic stress tolerance, nitrogen use efficiency, biomass, vigor or yield of the plant.

Thus, according to an aspect of the invention there is provided a method of improving abiotic stress tolerance, nitrogen use efficiency, biomass, vigor or yield of a plant, the method comprising expressing within the plant an exogenous polynucleotide having a nucleic acid sequence at least, 80%, 85%, 90%, 95% or even 100% identical to SEQ ID NOs: 101-216, 217-222, 223-227, 264-416 (Mature all upregulated sequences and homologs of Tables 1-8), wherein said nucleic acid sequence is capable of regulating abiotic stress tolerance of the plant, thereby improving abiotic stress tolerance, nitrogen use efficiency, biomass, vigor or yield of the plant

The phrase “abiotic stress” as used herein refers to any adverse effect on metabolism, growth, viability and/or reproduction of a plant. Abiotic stress can be induced by any of suboptimal environmental growth conditions such as, for example, water deficit or drought, flooding, freezing, low or high temperature, strong winds, heavy metal toxicity, anaerobiosis, high or low nutrient levels (e.g. nutrient deficiency), high or low salt levels (e.g. salinity), atmospheric pollution, high or low light intensities (e.g. insufficient light) or UV irradiation. Abiotic stress may be a short term effect (e.g. acute effect, e.g. lasting for about a week) or alternatively may be persistent (e.g. chronic effect, e.g. lasting for example 10 days or more). The present invention contemplates situations in which there is a single abiotic stress condition or alternatively situations in which two or more abiotic stresses occur.

According to an exemplary embodiment the abiotic stress refers to salinity.

According to another exemplary embodiment the abiotic stress refers to drought.

According to yet another exemplary embodiment the abiotic stress refers to high temperature.

As used herein the phrase “abiotic stress tolerance” or ABST refers to the ability of a plant to endure an abiotic stress without exhibiting substantial physiological or physical damage (e.g. alteration in metabolism, growth, viability and/or reproducibility of the plant).

It will be appreciated that since genes that affect abiotic stress tolerance often modulate any one of root architecture; plant metabolic pathways which may affect nitrogen absorption or localization; and plant surface permeability, it is also suggested that the biomolecular sequences (i.e., nucleic acid and amino acid sequences) of the present invention may also regulate nitrogen use efficiency of the plant.

As used herein the phrase “nitrogen use efficiency (NUE)” refers to a measure of crop production per unit of nitrogen fertilizer input. Fertilizer use efficiency (FUE) is a measure of NUE. Crop production can be measured by biomass, vigor or yield. The plant's nitrogen use efficiency is typically a result of an alteration in at least one of the uptake, spread, absorbance, accumulation, relocation (within the plant) and use of nitrogen absorbed by the plant. Improved abiotic stress tolerance or NUE is with respect to that of a non-transgenic plant (i.e., lacking the transgene of the transgenic plant) of the same species and of the same developmental stage and grown under the same conditions.

As used herein the phrase “nitrogen-limiting conditions” refers to growth conditions which include a level (e.g., concentration) of nitrogen (e.g., ammonium or nitrate) applied which is below the level needed for optimal plant metabolism, growth, reproduction and/or viability.

As used herein the term/phrase “biomass”, “biomass of a plant” or “plant biomass” refers to the amount (e.g., measured in grams of air-dry tissue) of a tissue produced from the plant in a growing season. An increase in plant biomass can be in the whole plant or in parts thereof such as aboveground (e.g. harvestable) parts, vegetative biomass, roots and/or seeds.

As used herein the term/phrase “vigor”, “vigor of a plant” or “plant vigor” refers to the amount (e.g., measured by weight) of tissue produced by the plant in a given time. Increased vigor could determine or affect the plant yield or the yield per growing time or growing area. In addition, early vigor (e.g. seed and/or seedling) results in improved field stand.

As used herein the term/phrase “yield”, “yield of a plant” or “plant yield” refers to the amount (e.g., as determined by weight or size) or quantity (e.g., numbers) of tissues or organs produced per plant or per growing season. Increased yield of a plant can affect the economic benefit one can obtain from the plant in a certain growing area and/or growing time.

According to an exemplary embodiment the yield is measured by cellulose content.

According to another exemplary embodiment the yield is measured by oil content.

According to another exemplary embodiment the yield is measured by protein content.

According to another exemplary embodiment, the yield is measured by seed number per plant or part thereof (e.g., kernel).

A plant yield can be affected by various parameters including, but not limited to, plant biomass; plant vigor; plant growth rate; seed yield; seed or grain quantity; seed or grain quality; oil yield; content of oil, starch and/or protein in harvested organs (e.g., seeds or vegetative parts of the plant); number of flowers (e.g. florets) per panicle (e.g. expressed as a ratio of number of filled seeds over number of primary panicles); harvest index; number of plants grown per area; number and size of harvested organs per plant and per area; number of plants per growing area (e.g. density); number of harvested organs in field; total leaf area; carbon assimilation and carbon partitioning (e.g. the distribution/allocation of carbon within the plant); resistance to shade; number of harvestable organs (e.g. seeds), seeds per pod, weight per seed; and modified architecture [such as increase stalk diameter, thickness or improvement of physical properties (e.g. elasticity)].

As used herein the term “improving” or “increasing” refers to at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90% or greater increase in nitrogen use efficiency, in tolerance to abiotic stress, in yield, in biomass or in vigor of a plant, as compared to a native or wild-type plants [i.e., plants not genetically modified to express the bio-molecules (e.g., polynucleotides) of the invention, e.g., a non-transformed plant of the same species and of the same developmental stage which is grown under the same growth conditions as the transformed plant].

Improved plant abiotic stress tolerance is translated in the field into harvesting similar quantities of yield, while growing on less than optimal conditions (e.g., salinity, heat, cold, drought etc.) or harvesting higher yield when growing under optimal growth conditions.

Improved plant nitrogen use efficiency is translated in the field into either harvesting similar quantities of yield, while implementing less fertilizers, or increased yields gained by implementing the same levels of fertilizers. Thus, improved NUE or FUE has a direct effect on plant yield in the field. Likewise, improved ABST refers to harvesting similar quantities of yield, while negating the need for growth under regulated conditions such as in a green-house or under irrigation.

The term “plant” as used herein encompasses whole plants, ancestors and progeny of the plants and plant parts, including seeds, shoots, stems, roots (including tubers), and isolated plant cells, tissues and organs. The plant may be in any form including suspension cultures, embryos, meristematic regions, callus tissue, leaves, gametophytes, sporophytes, pollen, and microspores.

As used herein the phrase “plant cell” refers to plant cells which are derived and isolated from disintegrated plant cell tissue or plant cell cultures.

As used herein the phrase “plant cell culture” refers to any type of native (naturally occurring) plant cells, plant cell lines and genetically modified plant cells, which are not assembled to form a complete plant, such that at least one biological structure of a plant is not present. Optionally, the plant cell culture of this aspect of the present invention may comprise a particular type of a plant cell or a plurality of different types of plant cells. It should be noted that optionally plant cultures featuring a particular type of plant cell may be originally derived from a plurality of different types of such plant cells.

Any commercially or scientifically valuable plant is envisaged in accordance with these embodiments of the invention. Plants that are particularly useful in the methods of the invention include all plants which belong to the super family Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cyclonia oblonga, Cryptomeria japonica, Cymbopogon spp., Cynthea dealbata, Cyclonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalyptus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, maize, wheat, barley, rye, oat, peanut, pea, lentil and alfalfa, cotton, rapeseed, canola, pepper, sunflower, tobacco, eggplant, eucalyptus, a tree, an ornamental plant, a perennial grass and a forage crop. Alternatively algae and other non-Viridiplantae can be used for the methods of the present invention.

According to some embodiments of the invention, the plant used by the method of the invention is a crop plant including, but not limited to, cotton, Brassica vegetables, oilseed rape, sesame, olive tree, palm oil, banana, wheat, corn or maize, barley, alfalfa, peanuts, sunflowers, rice, oats, sugarcane, soybean, turf grasses, barley, rye, sorghum, sugar cane, chicory, lettuce, tomato, zucchini, bell pepper, eggplant, cucumber, melon, watermelon, beans, hibiscus, okra, apple, rose, strawberry, chile, garlic, pea, lentil, canola, mums, arabidopsis, broccoli, cabbage, beet, quinoa, spinach, squash, onion, leek, tobacco, potato, sugarbeet, papaya, pineapple, mango, Arabidopsis thaliana, and also plants used in horticulture, floriculture or forestry, such as, but not limited to, poplar, fir, eucalyptus, pine, an ornamental plant, a perennial grass and a forage crop, coniferous plants, moss, algae, as well as other plants listed in World Wide Web(dot) nationmaster(dot)com/encyclopedia/Plantae.

According to a specific embodiment of the present invention, the plant comprises corn.

According to a specific embodiment of the present invention, the plant comprises sorghum.

As used herein, the phrase “exogenous polynucleotide” refers to a heterologous nucleic acid sequence which may not be naturally expressed within the plant or which overexpression in the plant is desired. The exogenous polynucleotide may be introduced into the plant in a stable or transient manner, so as to produce a ribonucleic acid (RNA) molecule. It should be noted that the exogenous polynucleotide may comprise a nucleic acid sequence which is identical or partially homologous to an endogenous nucleic acid sequence of the plant.

As mentioned the present teachings are based on the identification of RNA interfering molecular sequences (dsRNA) which modulate abiotic stress tolerance of plants.

According to some embodiments of the present aspect of the invention, the exogenous polynucleotide encodes an RNA interfering molecule.

Since its initial implementation, remarkable progress has been made in plant genetic engineering, and successful enhancements of commercially important crop plants have been reported (e.g., corn, cotton, soybean, canola, tomato). RNA interference (RNAi) is a remarkably potent technique and has steadily been established as the leading method for specific down-regulation/silencing of a target gene, through manipulation of one of two small RNA molecules, microRNAs (miRNAs) or small interfering RNAs (siRNAs). Both miRNAs and siRNAs are oligonucleotides (20-24 bps, i.e., the mature molecule) processed from longer RNA precursors by Dicer-like ribonucleases, although the source of their precursors is different (i.e., local single RNA molecules with imperfect stem-loop structures for miRNA, and long, double-stranded precursors potentially from bimolecular duplexes for siRNA). Additional characteristics that differentiate miRNAs from siRNAs are their sequence conservation level between related organisms (high in miRNAs, low to non-existent in siRNAs), regulation of genes unrelated to their locus of origin (typical for miRNAs, infrequent in siRNAs) and the genetic requirements for their respective functions are somewhat dissimilar in many organisms (Jones-Rhoades et al., 2006, Ann Rev Plant Biol 57:19-53). Despite all their differences, miRNAs and siRNAs are overall chemically and functionally similar and both are incorporated into silencing complexes, wherein they can guide post-transcriptional repression of multiple target genes, and thus function catalytically. Thus, the exogenous polynucleotide encodes a dsRNA interfering molecule or a precursor thereof.

According to some embodiments the exogenous polynucleotide encodes a miRNA or a precursor thereof.

According to other embodiments the exogenous polynucleotide encodes a siRNA or a precursor thereof.

As used herein, the phrase “siRNA” (also referred to herein interchangeably as “small interfering RNA” or “silencing RNA”, is a class of double-stranded RNA molecules, 20-25 nucleotides in length. The most notable role of siRNA is its involvement in the RNA interference (RNAi) pathway, where it interferes with the expression of a specific gene.

The siRNA precursor relates to a long dsRNA structure (at least 90% complementarity) of at least 30 bp.

As used herein, the phrase “microRNA (also referred to herein interchangeably as “miRNA” or “miR”) or a precursor thereof” refers to a microRNA (miRNA) molecule acting as a post-transcriptional regulator. Typically, the miRNA molecules are RNA molecules of about 20 to 22 nucleotides in length which can be loaded into a RISC complex and which direct the cleavage of another RNA molecule, wherein the other RNA molecule comprises a nucleotide sequence essentially complementary to the nucleotide sequence of the miRNA molecule.

Typically, a miRNA molecule is processed from a “pre-miRNA” or as used herein a precursor of a pre-miRNA molecule by proteins, such as DCL proteins, present in any plant cell and loaded onto a RISC complex where it can guide the cleavage of the target RNA molecules.

Pre-microRNA molecules are typically processed from pri-microRNA molecules (primary transcripts). The single stranded RNA segments flanking the pre-microRNA are important for processing of the pri-miRNA into the pre-miRNA. The cleavage site appears to be determined by the distance from the stem-ssRNA junction (Han et al. 2006, Cell 125, 887-901, 887-901).

As used herein, a “pre-miRNA” molecule is an RNA molecule of about 100 to about 200 nucleotides, preferably about 100 to about 130 nucleotides which can adopt a secondary structure comprising a double stranded RNA stem and a single stranded RNA loop (also referred to as “hairpin”) and further comprising the nucleotide sequence of the miRNA (and its complement sequence) in the double stranded RNA stem. According to a specific embodiment, the miRNA and its complement are located about 10 to about 20 nucleotides from the free ends of the miRNA double stranded RNA stem. The length and sequence of the single stranded loop region are not critical and may vary considerably, e.g. between 30 and 50 nt in length. The complementarity between the miRNA and its complement need not be perfect and about 1 to 3 bulges of unpaired nucleotides can be tolerated. The secondary structure adopted by an RNA molecule can be predicted by computer algorithms conventional in the art such as mFOLD. The particular strand of the double stranded RNA stem from the pre-miRNA which is released by DCL activity and loaded onto the RISC complex is determined by the degree of complementarity at the 5′ end, whereby the strand which at its 5′ end is the least involved in hydrogen bounding between the nucleotides of the different strands of the cleaved dsRNA stem is loaded onto the RISC complex and will determine the sequence specificity of the target RNA molecule degradation. However, if empirically the miRNA molecule from a particular synthetic pre-miRNA molecule is not functional (because the “wrong” strand is loaded on the RISC complex), it will be immediately evident that this problem can be solved by exchanging the position of the miRNA molecule and its complement on the respective strands of the dsRNA stem of the pre-miRNA molecule. As is known in the art, binding between A and U involving two hydrogen bounds, or G and U involving two hydrogen bounds is less strong that between G and C involving three hydrogen bounds. Exemplary hairpin sequences are provided in Tables 1-8, below.

Naturally occurring miRNA molecules may be comprised within their naturally occurring pre-miRNA molecules but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds. Some pre-miRNA scaffolds may be preferred over others for their efficiency to be correctly processed into the designed microRNAs, particularly when expressed as a chimeric gene wherein other DNA regions, such as untranslated leader sequences or transcription termination and polyadenylation regions are incorporated in the primary transcript in addition to the pre-microRNA.

According to the present teachings, the dsRNA molecules may be naturally occurring or synthetic.

Basically, siRNA and miRNA behave the same. Each can cleave perfectly complementary mRNA targets and decrease the expression of partially complementary targets.

Thus, the present teachings contemplate expressing an exogenous polynucleotide having a nucleic acid sequence at least 80%, 85%, 90%, 91%, 92%, 93%, 94%, 95%, 96%, 97%, 98% 99% or 100% identical to SEQ ID NOs. 101-216, 217-222, 223-227, 264-416 (Tables 1-8), provided that they regulate abiotic stress tolerance (e.g., heat stress, drought or salinity). Assays for testing the efficacy of transgenes on abiotic stress tolerance are further described hereinbelow.

Alternatively or additionally, the present teachings contemplate expressing an exogenous polynucleotide having a nucleic acid sequence at least 65%, 50%, 75%, 80%, 85%, 90%, 91%, 92%, 93%, 94%, 95%, 96%, 97%, 98% 99% or 100% identical to SEQ ID NOs. 217-222, 417-421, 458-614 (hairpin sequences of Tables 1-8 representing the core maize genes which were upregulated), provided that they regulate abiotic stress tolerance (e.g., heat stress, drought or salinity).

Tables 1-8 below illustrate exemplary miRNA sequences and precursors thereof which over expression are associated with modulation of abiotic stress tolerance.

For example, dsRNA sequences which are up-regulated during salinity stress are listed in Tables 3, 4 and 7.

dsRNA sequences which are up-regulated during heat stress are listed in Tables 5 and 8.

dsRNA sequences which are up-regulated during drought are listed in Tables 1, 2 and 6.

Likewise, Tables 1-8 provide similarly acting siRNA sequences.

The present invention envisages the use of homologous and orthologous sequences of the above RNA interfering molecules. At the precursor level use of homologous sequences can be done to a much broader extend. Thus, in such precursor sequences the degree of homology may be lower in all those sequences not including the mature miRNA or siRNA segment therein.

As used herein, the phrase “stem-loop precursor” refers to a stem loop precursor RNA structure from which the miRNA can be processed. In the case of siRNA, the precursor is typically devoid of a stem-loop structure.

Pre-microRNA molecules are typically processed from pri-microRNA molecules (primary transcripts). The single stranded RNA segments flanking the pre-microRNA are important for processing of the pri-miRNA into the pre-miRNA. The cleavage site appears to be determined by the distance from the stem-ssRNA junction (Han et al. 2006, Cell 125, 887-901, 887-901).

As used herein, a “pre-miRNA” molecule is an RNA molecule of about 100 to about 200 nucleotides, preferably about 100 to about 130 nucleotides which can adopt a secondary structure comprising a double stranded RNA stem and a single stranded RNA loop (also referred to as “hairpin”) and further comprising the nucleotide sequence of the miRNA (and its complement sequence) in the double stranded RNA stem. According to a specific embodiment, the miRNA and its complement are located about 10 to about 20 nucleotides from the free ends of the miRNA double stranded RNA stem. The length and sequence of the single stranded loop region are not critical and may vary considerably, e.g. between 30 and 50 nt in length. The complementarity between the miRNA and its complement need not be perfect and about 1 to 3 bulges of unpaired nucleotides can be tolerated. The secondary structure adopted by an RNA molecule can be predicted by computer algorithms conventional in the art such as mFOLD. The particular strand of the double stranded RNA stem from the pre-miRNA which is released by DCL activity and loaded onto the RISC complex is determined by the degree of complementarity at the 5′ end, whereby the strand which at its 5′ end is the least involved in hydrogen bonding between the nucleotides of the different strands of the cleaved dsRNA stem is loaded onto the RISC complex and will determine the sequence specificity of the target RNA molecule for degradation. However, if empirically the miRNA molecule from a particular synthetic pre-miRNA molecule is not functional (because the “wrong” strand is loaded on the RISC complex), it will be immediately evident that this problem can be solved by exchanging the position of the miRNA molecule and its complement on the respective strands of the dsRNA stem of the pre-miRNA molecule. As is known in the art, binding between A and U involving two hydrogen bonds, or G and U involving two hydrogen bonds is less strong than between G and C involving three hydrogen bonds.

Thus, according to a specific embodiment, the exogenous polynucleotide encodes a stem-loop precursor of the nucleic acid sequence. Such a stem-loop precursor can be at least about 60%, at least about 65%, at least about 70%, at least about 75%, at least about 80%, at least about 85%, at least about 90%, at least about 95% or more identical to SEQ ID NOs: 417-421, 458-614 (homolog precursors which are upregulated as in Tables 1-8), provided that it regulates abiotic stress tolerance (e.g., drought, salinity or heat stress).

Identity (e.g., percent identity) can be determined using any homology comparison software, including for example, the BlastN software of the National Center of Biotechnology Information (NCBI) such as by using default parameters.

Homology (e.g., percent homology, identity+similarity) can be determined using any homology comparison software, including for example, the TBLASTN software of the National Center of Biotechnology Information (NCBI) such as by using default parameters.

According to some embodiments of the invention, the term “homology” or “homologous” refers to identity of two or more nucleic acid sequences; or identity of two or more amino acid sequences.

Homologous sequences include both orthologous and paralogous sequences. The term “paralogous” relates to gene-duplications within the genome of a species leading to paralogous genes. The term “orthologous” relates to homologous genes in different organisms due to ancestral relationship.

One option to identify orthologues in monocot plant species is by performing a reciprocal blast search. This may be done by a first blast involving blasting the sequence-of-interest against any sequence database, such as the publicly available NCBI database which may be found at: Hypertext Transfer Protocol://World Wide Web(dot) ncbi(dot)nlm(dot)nih(dot)gov. The blast results may be filtered. The full-length sequences of either the filtered results or the non-filtered results are then blasted back (second blast) against the sequences of the organism from which the sequence-of-interest is derived. The results of the first and second blasts are then compared. An orthologue is identified when the sequence resulting in the highest score (best hit) in the first blast identifies in the second blast the query sequence (the original sequence-of-interest) as the best hit. Using the same rational a paralogue (homolog to a gene in the same organism) is found. In case of large sequence families, the ClustalW program may be used [Hypertext Transfer Protocol://World Wide Web(dot)ebi(dot)ac(dot) uk/Tools/clustalw2/index(dot)html], followed by a neighbor-joining tree (Hypertext Transfer Protocol://en(dot)wikipedia(dot)org/wiki/Neighbor-joining) which helps visualizing the clustering.

The miRNA or precursor sequence can be provided to the plant as naked RNA or expressed from a nucleic acid expression construct, where it is operaly linked to a regulatory sequence.

Interestingly, while screening for RNAi regulatory sequences, the present inventors have identified a number of miRNA and siRNA sequences which have never been described before.

Thus, according to an aspect of the invention there is provided an isolated polynucleotide having a nucleic acid sequence at least 80%, 85% 90%, 91%, 92%, 93%, 94%, 95%, 96%, 97%, 98% 99% or 100% identical to SEQ ID NO: 16-113, 117-216 (Tables 1-8 predicted dsRNA which are either upregulated or downregulated), wherein said nucleic acid sequence is capable of regulating abiotic stress tolerance of a plant (e.g., salinity, drought or heat stress).

According to a specific embodiment, the isolated polynucleotide encodes a stem-loop precursor of the nucleic acid sequence.

According to a specific embodiment, the stem-loop precursor is at least about 60%, at least about 65%, at least about 70%, at least about 75%, at least about 80%, at least about 85%, at least about 90%, at least about 95% or more identical to the precursor sequence, provided that it is capable of regulating abiotic stress tolerance of a plant (e.g., salinity, drought or heat stress).

According to a specific embodiment, the stem-loop precursor is selected from the group of precursor sequences of SEQ ID NOs: 101-113 and 117-216 (mature of predicted upregulated).

According to a specific embodiment, the stem-loop precursor is selected from the group of precursor sequences of SEQ ID NOs: 16-100.

As mentioned, the present inventors have also identified RNAi sequences which are down regulated under abiotic stress conditions (e.g., salinity, drought or heat stress).

Thus, according to an aspect of the invention there is provided a method of improving abiotic stress tolerance, nitrogen use efficiency, biomass, vigor or yield of a plant, the method comprising expressing within the plant an exogenous polynucleotide which downregulates an activity or expression of a gene encoding an RNAi molecule having a nucleic acid sequence at least 90% homologous to the sequence selected from the group consisting of SEQ ID NOs: 1-100, 615-626, 639 (Tables 1-8 MATURE DOWN-REGULATED), thereby improving, abiotic stress tolerance, nitrogen use efficiency, biomass, vigor or yield of a plant. Precursor hairpin sequences of those miRs are provided in SEQ ID NOs: 627-638 and 640 and homologous sequences (i.e., at least 60%, at least about 65%, at least about 70%, at least about 75%, at least about 80%, at least about 85%, at least about 90%, at least about 95% or more identical to the precursor sequence).

There are various approaches to down regulate RNAi sequences.

As used herein the term “down-regulation” refers to reduced activity or expression of the dsRNA (at least 10%, 20%, 30%, 50%, 60%, 70%, 80%, 90% or 100% reduction in activity or expression) as compared to its activity or expression in a plant of the same species and the same developmental stage not expressing the exogenous polynucleotide.

Nucleic acid agents that down-regulate miR activity include, but are not limited to, a target mimic, a micro-RNA resistant gene and a miRNA inhibitor.

The target mimic or micro-RNA resistant target is essentially complementary to the microRNA provided that one or more of following mismatches are allowed:

(a) a mismatch between the nucleotide at the 5′ end of the microRNA and the corresponding nucleotide sequence in the target mimic or micro-RNA resistant target;

(b) a mismatch between any one of the nucleotides in position 1 to position 9 of the microRNA and the corresponding nucleotide sequence in the target mimic or micro-RNA resistant target; or

(c) three mismatches between any one of the nucleotides in position 12 to position 21 of the microRNA and the corresponding nucleotide sequence in the target mimic or micro-RNA resistant target provided that there are no more than two consecutive mismatches.

The target mimic RNA is essentially similar to the target RNA modified to render it resistant to miRNA induced cleavage, e.g. by modifying the sequence thereof such that a variation is introduced in the nucleotide of the target sequence complementary to the nucleotides 10 or 11 of the miRNA resulting in a mismatch.

Alternatively, a microRNA-resistant target may be implemented. Thus, a silent mutation may be introduced in the microRNA binding site of the target gene so that the DNA and resulting RNA sequences are changed in a way that prevents microRNA binding, but the amino acid sequence of the protein is unchanged. Thus, a new sequence can be synthesized instead of the existing binding site, in which the DNA sequence is changed, resulting in lack of miRNA binding to its target.

Tables 14-19 below provide non-limiting examples of target mimics and target resistant sequences that can be used to down-regulate the activity of the miRs/siRNAs of the invention.

According to a specific embodiment, the target mimic or micro-RNA resistant target is linked to the promoter naturally associated with the pre-miRNA recognizing the target gene and introduced into the plant cell. In this way, the miRNA target mimic or micro-RNA resistant target RNA will be expressed under the same circumstances as the miRNA and the target mimic or micro-RNA resistant target RNA will substitute for the non-target mimic/micro-RNA resistant target RNA degraded by the miRNA induced cleavage.

Non-functional miRNA alleles or miRNA resistant target genes may also be introduced by homologous recombination to substitute the miRNA encoding alleles or miRNA sensitive target genes.

While further reducing the present invention to practice, the present inventors have uncovered through extensive experimentation and screening genes that are targeted by the dsRNA sequences of the present teachings and suggest overexpressing these genes or sequences controlling same in the generation of transgenic plants having improved agricultural traits.

Thus, according to an aspect of the invention there is provided a method of improving abiotic stress tolerance, nitrogen use efficiency, biomass, vigor or yield of a plant, the method comprising expressing within the plant an exogenous polynucleotide encoding a polypeptide having an amino acid sequence at least 80% homologous to SEQ ID NOs: 1861-1869, 1892-1915, 1921-1924, 1931-1939, 1952-1963, 2010-2014, 2327-2355, 2763-3040, 3044-3163, 3175-3269, 3313-3323, 3458-3944 or 3950-3969 (targets of down-regulated miRs of Tables 1-8), wherein said polypeptide is capable of regulating abiotic stress tolerance of the plant, thereby improving abiotic stress tolerance, nitrogen use efficiency, biomass, vigor or yield of the plant.

Generally, the tables provided in the Examples section are to be considered an integral part of the specification.

As used herein a “target gene” refers to a gene that is processed by microRNA or siRNA activity. Typically the gene encodes a polypeptide which expression is downregulated due to microRNA/siRNA processing.

Target genes are typically identified using the WMD3 website (http://wmd3dotweigelworlddotorg/).

As mentioned, the method of the present invention is performed by expressing within a plant an exogenous polynucleotide encoding a target gene of the RNA interfering molecules uncovered by the present inventors, as explained below.

As used herein, the phrase “expressing within the plant an exogenous polynucleotide” refers to upregulating the expression level of an exogenous polynucleotide within the plant e.g., by introducing the exogenous polynucleotide into a plant or plant cell and expressing by recombinant means, as described in detail hereinbelow.

As used herein “expressing” refers to expression at the mRNA level (e.g., in case the target gene expresses an mRNA product but no protein or in the case of expressing the dsRNA) or at the polypeptide level of the desired exogenous polynucleotide.

As used herein, the phrase “exogenous polynucleotide” refers to a heterologous nucleic acid sequence which may not be naturally expressed within the plant or which overexpression in the plant is desired (i.e., overexpression of an endogenous gene). The exogenous polynucleotide may be introduced into the plant in a stable or transient manner, so as to produce a ribonucleic acid (RNA) molecule and/or a polypeptide molecule. The exogenous polynucleotide may comprise a nucleic acid sequence which is identical or partially homologous to an endogenous nucleic acid sequence expressed within the plant.

The term “endogenous” as used herein refers to any polynucleotide or polypeptide which is present and/or naturally expressed within a plant or a cell thereof.

As used herein the term “polynucleotide” refers to a single or double stranded nucleic acid sequence which is isolated and provided in the form of an RNA sequence, a complementary polynucleotide sequence (cDNA), a genomic polynucleotide sequence (e.g. sequence isolated from a chromosome) and/or a composite polynucleotide sequences (e.g., a combination of the above). This term includes polynucleotides and/or oligonucleotides derived from naturally occurring nucleic acid molecules (e.g., RNA or DNA), synthetic polynucleotide and/or oligonucleotide molecules composed of naturally occurring bases, sugars, and covalent internucleoside linkages (e.g., backbone), as well as synthetic polynucleotides and/or oligonucleotides having non-naturally occurring portions, which function similarly to the respective naturally occurring portions.

The term “isolated” refers to at least partially separated from the natural environment e.g., from a plant cell.

Nucleic acid sequences of the polypeptides of some embodiments of the invention may be optimized for expression in a specific plant host. Examples of such sequence modifications include, but are not limited to, an altered G/C content to more closely approach that typically found in the plant species of interest, and the removal of codons atypically found in the plant species commonly referred to as codon optimization.

The phrase “codon optimization” refers to the selection of appropriate DNA nucleotides for use within a structural gene or fragment thereof that approaches codon usage within the plant of interest. Therefore, an optimized gene or nucleic acid sequence refers to a gene in which the nucleotide sequence of a native or naturally occurring gene has been modified in order to utilize statistically-preferred or statistically-favored codons within the plant. The nucleotide sequence typically is examined at the DNA level and the coding region optimized for expression in the plant species determined using any suitable procedure, for example as described in Sardana et al. (1996, Plant Cell Reports 15:677-681). In this method, the standard deviation of codon usage, a measure of codon usage bias, may be calculated by first finding the squared proportional deviation of usage of each codon of the native gene relative to that of highly expressed plant genes, followed by a calculation of the average squared deviation. The formula used is: 1 SDCU=n=1N[(Xn−Yn)/Yn]2/N, where Xn refers to the frequency of usage of codon n in highly expressed plant genes, where Yn to the frequency of usage of codon n in the gene of interest and N refers to the total number of codons in the gene of interest. A table of codon usage from highly expressed genes of dicotyledonous plants is compiled using the data of Murray et al. (1989, Nuc Acids Res. 17:477-498).

One method of optimizing the nucleic acid sequence in accordance with the preferred codon usage for a particular plant cell type is based on the direct use, without performing any extra statistical calculations, of codon optimization tables such as those provided on-line at the Codon Usage Database through the NIAS (National Institute of Agrobiological Sciences) DNA bank in Japan (www.kazusa.or.jp/codon/). The Codon Usage Database contains codon usage tables for a number of different species, with each codon usage table having been statistically determined based on the data present in Genbank.

By using the above tables to determine the most preferred or most favored codons for each amino acid in a particular species (for example, rice), a naturally-occurring nucleotide sequence encoding a protein of interest can be codon optimized for that particular plant species. This is effected by replacing codons that may have a low statistical incidence in the particular species genome with corresponding codons, in regard to an amino acid, that are statistically more favored. However, one or more less-favored codons may be selected to delete existing restriction sites, to create new ones at potentially useful junctions (5′ and 3′ ends to add signal peptide or termination cassettes, internal sites that might be used to cut and splice segments together to produce a correct full-length sequence), or to eliminate nucleotide sequences that may negatively effect mRNA stability or expression.

The naturally-occurring encoding nucleotide sequence may already, in advance of any modification, contain a number of codons that correspond to a statistically-favored codon in a particular plant species. Therefore, codon optimization of the native nucleotide sequence may comprise determining which codons, within the native nucleotide sequence, are not statistically-favored with regards to a particular plant, and modifying these codons in accordance with a codon usage table of the particular plant to produce a codon optimized derivative. A modified nucleotide sequence may be fully or partially optimized for plant codon usage provided that the protein encoded by the modified nucleotide sequence is produced at a level higher than the protein encoded by the corresponding naturally occurring or native gene. Construction of synthetic genes by altering the codon usage is described in for example PCT Patent Application 93/07278.

Target genes which are contemplated according to the present teachings are provided in the polynucleotide sequences encoding polypeptides which comprise amino acid sequences as set forth in SEQ ID NO:1816-2014, 2183-2355, 2501-3970. However the present teachings also relate to orthologs or homologs at least about 60%, at least about 65%, at least about 70%, at least about 75%, at least about 80%, at least about 85%, at least about 90%, or at least about 95% or more identical or similar to SEQ ID NO: 1816-2014, 2183-2355, 2500-3969. Parameters for determining the level of identity are provided hereinbelow.

Alternatively or additionally, target genes which are contemplated according to the present teachings are provided in the polynucleotide sequences which comprise nucleic acid sequences as set forth in SEQ ID NO: 2015-2182, 2356-2499, 3970-5236. However the present teachings also relate to orthologs or homologs at least about 60%, at least about 65%, at least about 70%, at least about 75%, at least about 80%, at least about 85%, at least about 90%, or at least about 95% or more identical or similar to SEQ ID NO: 2015-2182, 2356-2499, 3970-5236 (Tables 20-22).

Homology (e.g., percent homology, identity+similarity) can be determined using any homology comparison software, including for example, the TBLASTN software of the National Center of Biotechnology Information (NCBI) such as by using default parameters, when starting from a polypeptide sequence; or the tBLASTX algorithm (available via the NCBI) such as by using default parameters, which compares the six-frame conceptual translation products of a nucleotide query sequence (both strands) against a protein sequence database.

According to some embodiments of the invention, the term “homology” or “homologous” refers to identity of two or more nucleic acid sequences; or identity of two or more amino acid sequences.

Homologous sequences include both orthologous and paralogous sequences. The term “paralogous” relates to gene-duplications within the genome of a species leading to paralogous genes. The term “orthologous” relates to homologous genes in different organisms due to ancestral relationship.

One option to identify orthologues in monocot plant species is by performing a reciprocal blast search. This may be done by a first blast involving blasting the sequence-of-interest against any sequence database, such as the publicly available NCBI database which may be found at: Hypertext Transfer Protocol://World Wide Web(dot) ncbi(dot)nlm(dot)nih(dot)gov. The blast results may be filtered. The full-length sequences of either the filtered results or the non-filtered results are then blasted back (second blast) against the sequences of the organism from which the sequence-of-interest is derived. The results of the first and second blasts are then compared. An orthologue is identified when the sequence resulting in the highest score (best hit) in the first blast identifies in the second blast the query sequence (the original sequence-of-interest) as the best hit. Using the same rational a paralogue (homolog to a gene in the same organism) is found. In case of large sequence families, the ClustalW program may be used [Hypertext Transfer Protocol://World Wide Web(dot)ebi(dot)ac(dot) uk/Tools/clustalw2/index(dot)html], followed by a neighbor-joining tree (Hypertext Transfer Protocol://en(dot)wikipedia(dot)org/wiki/Neighbor-joining) which helps visualizing the clustering.

As mentioned the present inventors have also identified genes which down-regulation may be done in order to improve their NUE, biomass, vigor, yield and abiotic stress tolerance.

Thus, according to an aspect of the invention there is provided a method of improving abiotic stress tolerance, nitrogen use efficiency, biomass, vigor or yield of a plant, the method comprising expressing within the plant an exogenous polynucleotide which downregulates an activity or expression of a polypeptide having an amino acid sequence at least 80%, 85%, 90%, 95%, or 100% homologous to SEQ ID NOs: 1816-1860, 1870-1891, 1916-1920, 1925-1930, 1940-1951, 1964-2009, 2183-2326, 2500-2762, 3041-3043, 3164-3174, 3270-3312, 3324-3457, 3945-3949 (targets of upregulated miRs shown in Tables 20-22), wherein said polypeptide is capable of regulating abiotic stress tolerance of the plant, thereby improving abiotic stress tolerance, nitrogen use efficiency, biomass, vigor or yield of the plant.

Down regulation of activity or expression is by at least 10%, 20%, 30%, 40%, 50%, 60%, 70%, 80%, 90% or even complete (100%) loss of activity or expression. Assays for measuring gene expression can be effected at the protein level (e.g., Western blot, ELISA) or at the mRNA level such as by RT-PCR.

According to a specific embodiment the amino acid sequence of the target gene is as set forth in SEQ ID NOs: 1816-1860, 1870-1891, 1916-1920, 1925-1930, 1940-1951, 1964-2009, 2183-2326, 2500-2762, 3041-3043, 3164-3174, 3270-3312, 3324-3457, 3945-3949 (targets of upregulated miRs, Tables 20-22).

Alternatively or additionally, the amino acid sequence of the target gene is encoded by a polynucleotide sequence as set forth in SEQ ID NOs: 2015-2052, 2062-2079, 2102-2105, 2110, 2117-2125, 2137-2177, 2356-2477, 3970-4184, 4421-4421, 4528-4538, 4625-4660, 4671-4786, 5214-5218 (targets of upregulated miRs, Tables 20-22).

Examples of polynucleotide downregulating agents that inhibit (also referred to herein as inhibitors or nucleic acid agents) the expression of a target gene are given below.

1. Polynucleotide-Based Inhibition of Gene Expression.

It will be appreciated, that any of these methods when specifically referring to downregulating expression/activity of the target genes can be used, at least in part, to downregulate expression or activity of endogenous RNA molecules.

i. Sense Suppression/Cosuppression

In some embodiments of the invention, inhibition of the expression of target gene may be obtained by sense suppression or cosuppression. For cosuppression, an expression cassette is designed to express an RNA molecule corresponding to all or part of a messenger RNA encoding a target gene in the “sense” orientation. Over-expression of the RNA molecule can result in reduced expression of the native gene. Accordingly, multiple plant lines transformed with the cosuppression expression cassette are screened to identify those that show the greatest inhibition of target gene expression.

The polynucleotide used for cosuppression may correspond to all or part of the sequence encoding the target gene, all or part of the 5′ and/or 3′ untranslated region of a target transcript, or all or part of both the coding sequence and the untranslated regions of a transcript encoding the target gene. In some embodiments where the polynucleotide comprises all or part of the coding region for the target gene, the expression cassette is designed to eliminate the start codon of the polynucleotide so that no protein product will be transcribed.

Cosuppression may be used to inhibit the expression of plant genes to produce plants having undetectable protein levels for the proteins encoded by these genes. See, for example, Broin, et al., (2002) Plant Cell 15:1517-1532. Cosuppression may also be used to inhibit the expression of multiple proteins in the same plant. Methods for using cosuppression to inhibit the expression of endogenous genes in plants are described in Flavell, et al., (1995) Proc. Natl. Acad. Sci. USA 91:3590-3596; Jorgensen, et al., (1996) Plant Mol. Biol. 31:957-973; Johansen and Carrington, (2001) Plant Physiol. 126:930-938; Broin, et al., (2002) Plant Cell 15:1517-1532; Stoutjesdijk, et al., (2002) Plant Physiol. 129:1723-1731; Yu, et al., (2003) Phytochemistry 63:753-763; and U.S. Pat. Nos. 5,035,323, 5,283,185 and 5,952,657; each of which is herein incorporated by reference. The efficiency of cosuppression may be increased by including a poly-dt region in the expression cassette at a position 3′ to the sense sequence and 5′ of the polyadenylation signal. See, US Patent Publication Number 20020058815, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,185 and 5,035,323; herein incorporated by reference.

Transcriptional gene silencing (TGS) may be accomplished through use of hpRNA constructs wherein the inverted repeat of the hairpin shares sequence identity with the promoter region of a gene to be silenced. Processing of the hpRNA into short RNAs which can interact with the homologous promoter region may trigger degradation or methylation to result in silencing. (Aufsatz, et al., (2002) PNAS 99(4):16499-16506; Mette, et al., (2000) EMBO J. 19(19):5194-5201)

ii. Antisense Suppression

In some embodiments of the invention, inhibition of the expression of the target gene may be obtained by antisense suppression. For antisense suppression, the expression cassette is designed to express an RNA molecule complementary to all or part of a messenger RNA encoding the target gene. Over-expression of the antisense RNA molecule can result in reduced expression of the native gene. Accordingly, multiple plant lines transformed with the antisense suppression expression cassette are screened to identify those that show the greatest inhibition of target gene expression.

The polynucleotide for use in antisense suppression may correspond to all or part of the complement of the sequence encoding the target gene, all or part of the complement of the 5′ and/or 3′ untranslated region of the target gene transcript, or all or part of the complement of both the coding sequence and the untranslated regions of a transcript encoding the target gene. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. Furthermore, portions of the antisense nucleotides may be used to disrupt the expression of the target gene. Generally, sequences of at least 50 nucleotides, 100 nucleotides, 200 nucleotides, 300, 500, 550, 500, 550 or greater may be used. Methods for using antisense suppression to inhibit the expression of endogenous genes in plants are described, for example, in Liu, et al., (2002) Plant Physiol. 129:1732-1753 and U.S. Pat. No. 5,759,829, which is herein incorporated by reference. Efficiency of antisense suppression may be increased by including a poly-dt region in the expression cassette at a position 3′ to the antisense sequence and 5′ of the polyadenylation signal. See, US Patent Publication Number 20020058815.

iii. Double-Stranded RNA Interference

In some embodiments of the invention, inhibition of the expression of a target gene may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of target gene expression. Methods for using dsRNA interference to inhibit the expression of endogenous plant genes are described in Waterhouse, et al., (1998) Proc. Natl. Acad. Sci. USA 95:13959-13965, Liu, et al., (2002) Plant Physiol. 129:1732-1753, and WO 99/59029, WO 99/53050, WO 99/61631, and WO 00/59035;

iv. Hairpin RNA Interference and Intron-Containing Hairpin RNA Interference

In some embodiments of the invention, inhibition of the expression of one or more target gene may be obtained by hairpin RNA (hpRNA) interference or intron-containing hairpin RNA (ihpRNA) interference. These methods are highly efficient at downregulating the expression of endogenous genes. See, Waterhouse and Helliwell, (2003) Nat. Rev. Genet. 5:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz, (2000) Proc. Natl. Acad. Sci. USA 97:5985-5990; Stoutjesdijk, et al., (2002) Plant Physiol. 129:1723-1731; and Waterhouse and Helliwell, (2003) Nat. Rev. Genet. 5:29-38. Methods for using hpRNA interference to inhibit or silence the expression of genes are described, for example, in Chuang and Meyerowitz, (2000) Proc. Natl. Acad. Sci. USA 97:5985-5990; Stoutjesdijk, et al., (2002) Plant Physiol. 129:1723-1731; Waterhouse and Helliwell, (2003) Nat. Rev. Genet. 5:29-38; Pandolfini, et al., BMC Biotechnology 3:7, and US Patent Publication Number 20030175965; each of which is herein incorporated by reference. A transient assay for the efficiency of hpRNA constructs to silence gene expression in vivo has been described by Panstruga, et al., (2003) Mol. Biol. Rep. 30:135-150, herein incorporated by reference.

ForihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 507:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 507:319-320; Wesley, et al., (2001) Plant J. 27:581-590; Wang and Waterhouse, (2001) Curr. Opin. Plant Biol. 5:156-150; Waterhouse and Helliwell, (2003) Nat. Rev. Genet. 5:29-38; Helliwell and Waterhouse, (2003) Methods 30:289-295, and US Patent Publication Number 20030180955, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00905, herein incorporated by reference.

v. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for target gene). Methods of using amplicons to inhibit the expression of endogenous plant genes are described, for example, in Angell and Baulcombe, (1997) EMBO J. 16:3675-3685, Angell and Baulcombe, (1999) Plant J. 20:357-362, and U.S. Pat. No. 6,656,805, each of which is herein incorporated by reference.

vi. Ribozymes

In some embodiments, the polynucleotide expressed by the expression cassette of the invention is catalytic RNA or has ribozyme activity specific for the messenger RNA of target gene. Thus, the polynucleotide causes the degradation of the endogenous messenger RNA, resulting in reduced expression of the target gene. This method is described, for example, in U.S. Pat. No. 5,987,071, herein incorporated by reference.

2. Gene Disruption

In some embodiments of the present invention, the activity of a miRNA or a target gene is reduced or eliminated by disrupting the gene encoding the target polypeptide. The gene encoding the target polypeptide may be disrupted by any method known in the art. For example, in one embodiment, the gene is disrupted by transposon tagging. In another embodiment, the gene is disrupted by mutagenizing plants using random or targeted mutagenesis, and selecting for plants that have reduced response regulator activity.

Recombinant expression is effected by cloning the nucleic acid of interest (e.g., miRNA, target gene, silencing agent etc) into a nucleic acid expression construct under the expression of a plant promoter.

In other embodiments of the invention, synthetic single stranded nucleic acids are used as miRNA inhibitors. A miRNA inhibitor is typically between about 17 to 25 nucleotides in length and comprises a 5′ to 3′ sequence that is at least 90% complementary to the 5′ to 3′ sequence of a mature miRNA. In certain embodiments, a miRNA inhibitor molecule is 17, 18, 19, 20, 21, 22, 23, 24, or 25 nucleotides in length, or any range derivable therein. Moreover, a miRNA inhibitor has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA.

As mentioned, the polynucleotide sequences of the invention can be provided to the plant as naked RNA or expressed from a nucleic acid expression construct, where it is operaly linked to a regulatory sequence.

According to a specific embodiment of the invention, there is provided a nucleic acid construct comprising a nucleic acid sequence encoding a miRNA or siRNA or a precursor thereof as described herein, said nucleic acid sequence being under a transcriptional control of a regulatory sequence such as a fiber-cell specific promoter.

Alternatively or additionally, there is provided a nucleic acid construct comprising a nucleic acid sequence encoding an inhibitor of the miRNA or siRNA sequences as described herein (e.g., target mimic, miR resistant target or miR inhibitor), said nucleic acid sequence being under a transcriptional control of a regulatory sequence such as a tissue (e.g., root) specific promoter.

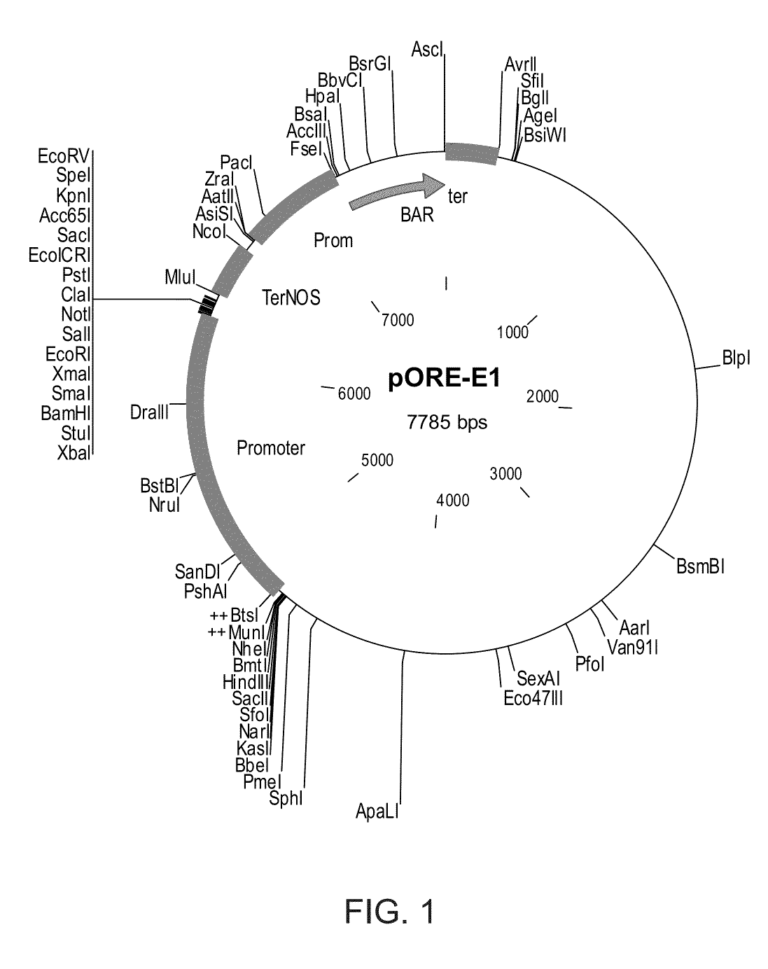

An exemplary nucleic acid construct which can be used for plant transformation include, the pORE E2 binary vector (FIG. 1) in which the relevant polynucleotide sequence is ligated under the transcriptional control of a promoter.

A coding nucleic acid sequence is “operably linked” or “transcriptionally linked to a regulatory sequence (e.g., promoter)” if the regulatory sequence is capable of exerting a regulatory effect on the coding sequence linked thereto. Thus the regulatory sequence controls the transcription of the miRNA or precursor thereof.

The term “regulatory sequence”, as used herein, means any DNA, that is involved in driving transcription and controlling (i.e., regulating) the timing and level of transcription of a given DNA sequence, such as a DNA coding for a miRNA or siRNA, precursor or inhibitor of same. For example, a 5′ regulatory region (or “promoter region”) is a DNA sequence located upstream (i.e., 5′) of a coding sequence and which comprises the promoter and the 5′-untranslated leader sequence. A 3′ regulatory region is a DNA sequence located downstream (i.e., 3′) of the coding sequence and which comprises suitable transcription termination (and/or regulation) signals, including one or more polyadenylation signals.

For the purpose of the invention, the promoter is a plant-expressible promoter. As used herein, the term “plant-expressible promoter” means a DNA sequence which is capable of controlling (initiating) transcription in a plant cell. This includes any promoter of plant origin, but also any promoter of non-plant origin which is capable of directing transcription in a plant cell, i.e., certain promoters of viral or bacterial origin Thus, any suitable promoter sequence can be used by the nucleic acid construct of the present invention. According to some embodiments of the invention, the promoter is a constitutive promoter, a tissue-specific promoter or an inducible promoter (e.g. an abiotic stress-inducible promoter).

Suitable constitutive promoters include, for example, hydroperoxide lyase (HPL) promoter, CaMV 35S promoter (Odell et al, Nature 313:810-812, 1985); maize Ubi 1 (Christensen et al., Plant Sol. Biol. 18:675-689, 1992); rice actin (McElroy et al., Plant Cell 2:163-171, 1990); pEMU (Last et al, Theor. Appl. Genet. 81:581-588, 1991); CaMV 19S (Nilsson et al, Physiol. Plant 100:456-462, 1997); GOS2 (de Pater et al, Plant J November; 2(6):837-44, 1992); ubiquitin (Christensen et al, Plant MoI. Biol. 18: 675-689, 1992); Rice cyclophilin (Bucholz et al, Plant MoI Biol. 25(5):837-43, 1994); Maize H3 histone (Lepetit et al, MoI. Gen. Genet. 231: 276-285, 1992); Actin 2 (An et al, Plant J. 10(1); 107-121, 1996) and Synthetic Super MAS (Ni et al., The Plant Journal 7: 661-76, 1995). Other constitutive promoters include those in U.S. Pat. Nos. 5,659,026, 5,608,149; 5,608,144; 5,604,121; 5,569,597: 5,466,785; 5,399,680; 5,268,463; and 5,608,142.

Suitable tissue-specific promoters include, but not limited to, leaf-specific promoters [such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant MoI. Biol. 23:1129-1138, 1993; and Matsuoka et al., Proc. Natl. Acad. Sci. USA 90:9586-9590, 1993], seed-preferred promoters [e.g., from seed specific genes (Simon, et al., Plant MoI. Biol. 5. 191, 1985; Scofield, et al., J. Biol. Chem. 262: 12202, 1987; Baszczynski, et al., Plant MoI. Biol. 14: 633, 1990), Brazil Nut albumin (Pearson' et al., Plant MoI. Biol. 18: 235-245, 1992), legumin (Ellis, et al. Plant MoI. Biol. 10: 203-214, 1988), Glutelin (rice) (Takaiwa, et al., MoI. Gen. Genet. 208: 15-22, 1986; Takaiwa, et al., FEBS Letts. 221: 43-47, 1987), Zein (Matzke et al., Plant MoI Biol, 143)323-32 1990), napA (Stalberg, et al., Planta 199: 515-519, 1996), Wheat SPA (Albanietal, Plant Cell, 9: 171-184, 1997), sunflower oleosin (Cummins, et al, Plant MoI. Biol. 19: 873-876, 1992)], endosperm specific promoters [e.g., wheat LMW and HMW, glutenin-1 (MoI Gen Genet. 216:81-90, 1989; NAR 17:461-2), wheat a, b and g gliadins (EMBO3: 1409-15, 1984), Barley ltrl promoter, barley Bl, C, D hordein (Theor Appl Gen 98:1253-62, 1999; Plant J 4:343-55, 1993; MoI Gen Genet. 250:750-60, 1996), Barley DOF (Mena et al., The Plant Journal, 116(1): 53-62, 1998), Biz2 (EP99106056.7), Synthetic promoter (Vicente-Carbajosa et al., Plant J. 13: 629-640, 1998), rice prolamin NRP33, rice-globulin GIb-I (Wu et al., Plant Cell Physiology 39(8) 885-889, 1998), rice alpha-globulin REB/OHP-1 (Nakase et al. Plant MoI. Biol. 33: 513-S22, 1997), rice ADP-glucose PP (Trans Res 6:157-68, 1997), maize ESR gene family (Plant J 12:235-46, 1997), sorghum gamma-kafirin (PMB 32:1029-35, 1996); e.g., the Napin promoter], embryo specific promoters [e.g., rice OSH1 (Sato et al, Proc. Natl. Acad. Sci. USA, 93: 8117-8122), KNOX (Postma-Haarsma et al, Plant MoI. Biol. 39:257-71, 1999), rice oleosin (Wu et at, J. Biochem., 123:386, 1998)], and flower-specific promoters [e.g., AtPRP4, chalene synthase (chsA) (Van der Meer, et al., Plant MoI. Biol. 15, 95-109, 1990), LAT52 (Twell et al., MoI. Gen Genet. 217:240-245; 1989), apetala-3]. Also contemplated are root-specific promoters such as the ROOTP promoter described in Vissenberg K, et al. Plant Cell Physiol. 2005 January; 46(1):192-200.

The nucleic acid construct of some embodiments of the invention can further include an appropriate selectable marker and/or an origin of replication.

The nucleic acid construct of some embodiments of the invention can be utilized to stably or transiently transform plant cells. In stable transformation, the exogenous polynucleotide is integrated into the plant genome and as such it represents a stable and inherited trait. In transient transformation, the exogenous polynucleotide is expressed by the cell transformed but it is not integrated into the genome and as such it represents a transient trait.

When naked RNA or DNA is introduced into a cell, the polynucleotides may be synthesized using any method known in the art, including either enzymatic syntheses or solid-phase syntheses. These are especially useful in the case of short polynucleotide sequences with or without modifications as explained above. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

There are various methods of introducing foreign genes into both monocotyledonous and dicotyledonous plants (Potrykus, L, Annu. Rev. Plant. Physiol, Plant. MoI. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer (e.g., T-DNA using Agrobacterium tumefaciens or Agrobacterium rhizogenes); see for example, Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S, and Arntzen, C. J., Butterworth Publishers, Boston, Mass. (1989) p. 93-112.

(ii) Direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. See, e.g., Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

According to a specific embodiment of the present invention, the exogenous polynucleotide is introduced into the plant by infecting the plant with a bacteria, such as using a floral dip transformation method (as described in further detail in Example 5, of the Examples section which follows).

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. For this reason it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by the present invention.

Transient transformation can be effected by any of the direct DNA transfer methods described above or by viral infection using modified plant viruses.

Viruses that have been shown to be useful for the transformation of plant hosts include CaMV, Tobacco mosaic virus (TMV), brome mosaic virus (BMV) and Bean Common Mosaic Virus (BV or BCMV). Transformation of plants using plant viruses is described in U.S. Pat. No. 4,855,237 (bean golden mosaic virus; BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants are described in WO 87/06261. According to some embodiments of the invention, the virus used for transient transformations is avirulent and thus is incapable of causing severe symptoms such as reduced growth rate, mosaic, ring spots, leaf roll, yellowing, streaking, pox formation, tumor formation and pitting. A suitable avirulent virus may be a naturally occurring avirulent virus or an artificially attenuated virus. Virus attenuation may be effected by using methods well known in the art including, but not limited to, sub-lethal heating, chemical treatment or by directed mutagenesis techniques such as described, for example, by Kurihara and Watanabe (Molecular Plant Pathology 4:259-269, 2003), Galon et al. (1992), Atreya et al. (1992) and Huet et al. (1994).

Suitable virus strains can be obtained from available sources such as, for example, the American Type culture Collection (ATCC) or by isolation from infected plants. Isolation of viruses from infected plant tissues can be effected by techniques well known in the art such as described, for example by Foster and Tatlor, Eds. “Plant Virology Protocols From Virus Isolation to Transgenic Resistance (Methods in Molecular Biology (Humana Pr), VoI 81)”, Humana Press, 1998. Briefly, tissues of an infected plant believed to contain a high concentration of a suitable virus, preferably young leaves and flower petals, are ground in a buffer solution (e.g., phosphate buffer solution) to produce a virus infected sap which can be used in subsequent inoculations.

Construction of plant RNA viruses for the introduction and expression of non-viral exogenous polynucleotide sequences in plants is demonstrated by the above references as well as by Dawson, W. O. et al, Virology (1989) 172:285-292; Takamatsu et al. EMBO J. (1987) 6:307-311; French et al. Science (1986) 231:1294-1297; Takamatsu et al. FEBS Letters (1990) 269:73-76; and U.S. Pat. No. 5,316,931.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat proteins which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired sequence.

In addition to the above, the nucleic acid molecule of the present invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference.

Regardless of the method of transformation, propagation or regeneration, the present invention also contemplates a transgenic plant exogenously expressing the polynucleotide of the invention.

According to a specific embodiment, the transgenic plant exogenously expresses a polynucleotide having a nucleic acid sequence at least 80%, 85%, 90%, 95% or even 100% identical to SEQ ID NOs: 1-216, 217-222, 223-227, 264-416, 417-421, 458-614, 615-626, 627-638, 639 or 640 (Tables 1-8), wherein said nucleic acid sequence is capable of regulating abiotic stress tolerance (e.g., salinity, heat stress or drought) of the plant.

According to further embodiments, the exogenous polynucleotide encodes a precursor of said nucleic acid sequence.

According to yet further embodiments, the stem-loop precursor is at least 60%, 65%, 70%, 75%, 80%, 85%, 90%, 95% or even 100% identical to SEQ ID NO: 217-222, 417-421, 458-614, 627-638 or 640 (precursor sequences of Tables 1-8) but importantly comprises a sequence that is at least 90% identical to SEQ ID NOs: 1-216, 217-222, 223-227, 264-416, 615-626 or 639 (Tables 1-8 including all the mature sequences). More specifically the exogenous polynucleotide is selected from the group consisting of SEQ ID NO: 1-216, 217-222, 223-227, 264-416, 417-421, 458-614, 615-626, 627-638, 639 or 640.

Alternatively, there is provided a transgenic plant exogenously expressing a polynucleotide which downregulates an activity or expression of a gene encoding an RNAi molecule having a nucleic acid sequence selected from the group consisting of SEQ ID NOs: 1-100, 615-626, 627-638, 639 or 640.

More specifically, the transgenic plant expresses the nucleic acid agent of Tables 14-19. Even more specifically, to improve the agricultural traits of the transgenic plant, it expresses a nucleic acid agent of Tables 14, 15a, 16a and 17-19.

Also provided are transgenic plants over expressing the target gene of the invention such as exogenously expressing polypeptide sequences which comprise amino acid sequence selected from the group consisting of SEQ ID NOs: 1816-1860, 1870-1891, 1916-1920, 1925-1930, 1940-1951, 1964-2009, 2183-2326, 2500-2762, 3041-3043, 3164-3174, 3270-3312, 3324-3457, 3945-3949 (targets of upregulated dsRNAs of Tables 20-22) or homologs/orthologs of same (at least about 60%, 65%, 70%, 75%, 80%, 85%, 90%, 95% or higher level of homology as described above).

Accordingly, the present teachings also contemplate nucleic acid expression constructs and plants which comprise the same expressing polynucleotide sequences at least about 60%, 65%, 70%, 75%, 80%, 85%, 90%, 95% or higher level of identity to SEQ ID NOs: 2015-2052, 2062-2079, 2102-2105, 2110, 2117-2125, 2137-2177, 2355-2477, 3970-4184, 4419-4421, 4528-4539, 4625-4660, 4671-4786, 5214-5218 (targets of upregulated dsRNAs of Tables 20-22).

Also contemplated are transgenic plants which express any of the polynucleotide or polypeptide sequences of the present invention (SEQ ID NOs: 1-640, 877-886, 893-913, 932-1012, 1226-1535, 1617-5237 and homologs thereof). This is important for analyzing the significance of those sequences in regulating abiotic stress tolerance and biomass, NUE, vigor or yield.

Also contemplated are hybrids of the above described transgenic plants. A “hybrid plant” refers to a plant or a part thereof resulting from a cross between two parent plants, wherein one parent is a genetically engineered plant of the invention (transgenic plant expressing an exogenous RNAi sequence or a precursor thereof). Such a cross can occur naturally by, for example, sexual reproduction, or artificially by, for example, in vitro nuclear fusion. Methods of plant breeding are well-known and within the level of one of ordinary skill in the art of plant biology.

Since abiotic stress tolerance, nitrogen use efficiency as well as yield, vigor or biomass of the plant can involve multiple genes acting additively or in synergy (see, for example, in Quesda et al., Plant Physiol. 130:951-063, 2002), the invention also envisages expressing a plurality of exogenous polynucleotides in a single host plant to thereby achieve superior effect on abiotic stress tolerance, efficiency of nitrogen use, yield, vigor and biomass of the plant.

Expressing a plurality of exogenous polynucleotides in a single host plant can be effected by co-introducing multiple nucleic acid constructs, each including a different exogenous polynucleotide, into a single plant cell. The transformed cell can then be regenerated into a mature plant using the methods described hereinabove. Alternatively, expressing a plurality of exogenous polynucleotides in a single host plant can be effected by co-introducing into a single plant-cell a single nucleic-acid construct including a plurality of different exogenous polynucleotides. Such a construct can be designed with a single promoter sequence which can transcribe a polycistronic messenger RNA including all the different exogenous polynucleotide sequences. Alternatively, the construct can include several promoter sequences each linked to a different exogenous polynucleotide sequence.

The plant cell transformed with the construct including a plurality of different exogenous polynucleotides can be regenerated into a mature plant, using the methods described hereinabove.

Alternatively, expressing a plurality of exogenous polynucleotides can be effected by introducing different nucleic acid constructs, including different exogenous polynucleotides, into a plurality of plants. The regenerated transformed plants can then be cross-bred and resultant progeny selected for superior yield or fiber traits as described above, using conventional plant breeding techniques.

Expression of the miRNAs/siRNAs of the present invention or precursors thereof can be qualified using methods which are well known in the art such as those involving gene amplification e.g., PCR or RT-PCR or Northern blot or in-situ hybridization.

According to some embodiments of the invention, the plant expressing the exogenous polynucleotide(s) is grown under stress (abiotic) or normal conditions (e.g., biotic conditions and/or abiotic conditions with sufficient water, optimal temperature and salt). Such conditions, which depend on the plant being grown, are known to those skilled in the art of agriculture, and are further, described above. Examples 7-9 hereinbelow provides specific assays for measuring abiotic stress tolerance.

According to some embodiments of the invention, the method further comprises growing the plant expressing the exogenous polynucleotide(s) under abiotic stress or nitrogen limiting conditions. Non-limiting examples of abiotic stress conditions include, water deprivation, drought, excess of water (e.g., flood, waterlogging), freezing, low temperature, high temperature, strong winds, heavy metal toxicity, anaerobiosis, nutrient deficiency, nutrient excess, salinity, atmospheric pollution, intense light, insufficient light, or UV irradiation, etiolation and atmospheric pollution.

Thus, the invention encompasses plants exogenously expressing the polynucleotide(s), the nucleic acid constructs of the invention.

Methods of determining the level in the plant of the RNA transcribed from the exogenous polynucleotide are well known in the art and include, for example, Northern blot analysis, reverse transcription polymerase chain reaction (RT-PCR) analysis (including quantitative, semi-quantitative or real-time RT-PCR) and RNA-m situ hybridization.

The sequence information and annotations uncovered by the present teachings can be harnessed in favor of classical breeding. Thus, sub-sequence data of those polynucleotides described above, can be used as markers for marker assisted selection (MAS), in which a marker is used for indirect selection of a genetic determinant or determinants of a trait of interest (e.g., tolerance to abiotic stress). Nucleic acid data of the present teachings (DNA or RNA sequence) may contain or be linked to polymorphic sites or genetic markers on the genome such as restriction fragment length polymorphism (RFLP), microsatellites and single nucleotide polymorphism (SNP), DNA fingerprinting (DFP), amplified fragment length polymorphism (AFLP), expression level polymorphism, and any other polymorphism at the DNA or RNA sequence.

Examples of marker assisted selections include, but are not limited to, selection for a morphological trait (e.g., a gene that affects form, coloration, male sterility or resistance such as the presence or absence of awn, leaf sheath coloration, height, grain color, aroma of rice); selection for a biochemical trait (e.g., a gene that encodes a protein that can be extracted and observed; for example, isozymes and storage proteins); selection for a biological trait (e.g., pathogen races or insect biotypes based on host pathogen or host parasite interaction can be used as a marker since the genetic constitution of an organism can affect its susceptibility to pathogens or parasites).

The polynucleotides described hereinabove can be used in a wide range of economical plants, in a safe and cost effective manner.

Plant lines exogenously expressing the polynucleotide of the invention can be screened to identify those that show the greatest increase of the desired plant trait.

Thus, according to an additional embodiment of the present invention, there is provided a method of evaluating a trait of a plant, the method comprising: (a) expressing in a plant or a portion thereof the nucleic acid construct; and (b) evaluating a trait of a plant as compared to a wild type plant of the same type; thereby evaluating the trait of the plant.

Thus, the effect of the transgene (the exogenous polynucleotide) on different plant characteristics may be determined any method known to one of ordinary skill in the art.

Thus, for example, tolerance to abiotic stress conditions may be compared in transformed plants {i.e., expressing the transgene) compared to non-transformed (wild type) plants exposed to the same stress conditions (e.g. water deprivation, salt stress e.g. salinity, suboptimal temperature, osmotic stress, and the like), using the following assays.

Methods of qualifying plants as being tolerant or having improved tolerance to abiotic stress or limiting nitrogen levels are well known in the art and are further described hereinbelow.

Fertilizer use efficiency—To analyze whether the transgenic plants are more responsive to fertilizers, plants are grown in agar plates or pots with a limited amount of fertilizer, as described, for example, in Yanagisawa et al (Proc Natl Acad Sci USA. 2004; 101:7833-8). The plants are analyzed for their overall size, time to flowering, yield, protein content of shoot and/or grain. The parameters checked are the overall size of the mature plant, its wet and dry weight, the weight of the seeds yielded, the average seed size and the number of seeds produced per plant. Other parameters that may be tested are: the chlorophyll content of leaves (as nitrogen plant status and the degree of leaf verdure is highly correlated), amino acid and the total protein content of the seeds or other plant parts such as leaves or shoots, oil content, etc. Similarly, instead of providing nitrogen at limiting amounts, phosphate or potassium can be added at increasing concentrations. Again, the same parameters measured are the same as listed above. In this way, nitrogen use efficiency (NUE), phosphate use efficiency (PUE) and potassium use efficiency (KUE) are assessed, checking the ability of the transgenic plants to thrive under nutrient restraining conditions.

Nitrogen use efficiency—To analyze whether the transgenic plants (e.g., Arabidopsis plants) are more responsive to nitrogen, plant are grown in 0.75-3 millimolar (mM, nitrogen deficient conditions) or 6-10 mM (optimal nitrogen concentration). Plants are allowed to grow for additional 25 days or until seed production. The plants are then analyzed for their overall size, time to flowering, yield, protein content of shoot and/or grain/seed production. The parameters checked can be the overall size of the plant, wet and dry weight, the weight of the seeds yielded, the average seed size and the number of seeds produced per plant. Other parameters that may be tested are: the chlorophyll content of leaves (as nitrogen plant status and the degree of leaf greenness is highly correlated), amino acid and the total protein content of the seeds or other plant parts such as leaves or shoots and oil content. Transformed plants not exhibiting substantial physiological and/or morphological effects, or exhibiting higher measured parameters levels than wild-type plants, are identified as nitrogen use efficient plants.

Nitrogen Use efficiency assay using plantlets—The assay is done according to Yanagisawa-S. et al. with minor modifications (“Metabolic engineering with Dof1 transcription factor in plants: Improved nitrogen assimilation and growth under low-nitrogen conditions” Proc. Natl. Acad. Sci. USA 101, 7833-7838). Briefly, transgenic plants which are grown for 7-10 days in 0.5×MS [Murashige-Skoog] supplemented with a selection agent are transferred to two nitrogen-limiting conditions: MS media in which the combined nitrogen concentration (NH₄NO₃ and KNO₃) was 0.75 mM (nitrogen deficient conditions) or 6-15 mM (optimal nitrogen concentration). Plants are allowed to grow for additional 30-40 days and then photographed, individually removed from the Agar (the shoot without the roots) and immediately weighed (fresh weight) for later statistical analysis. Constructs for which only T1 seeds are available are sown on selective media and at least 20 seedlings (each one representing an independent transformation event) are carefully transferred to the nitrogen-limiting media. For constructs for which T2 seeds are available, different transformation events are analyzed. Usually, 20 randomly selected plants from each event are transferred to the nitrogen-limiting media allowed to grow for 3-4 additional weeks and individually weighed at the end of that period. Transgenic plants are compared to control plants grown in parallel under the same conditions. Mock-transgenic plants expressing the uidA reporter gene (GUS) under the same promoter or transgenic plants carrying the same promoter but lacking a reporter gene are used as control.

Nitrogen determination—The procedure for N (nitrogen) concentration determination in the structural parts of the plants involves the potassium persulfate digestion method to convert organic N to NO₃⁻ (Purcell and King 1996 Argon. J. 88:111-113, the modified Cd⁻ mediated reduction of NO₃⁻ to NO₂⁻ (Vodovotz 1996 Biotechniques 20:390-394) and the measurement of nitrite by the Griess assay (Vodovotz 1996, supra). The absorbance values are measured at 550 nm against a standard curve of NaNO₂. The procedure is described in details in Samonte et al. 2006 Agron. J. 98:168-176.

Tolerance to abiotic stress (e.g. tolerance to drought or salinity) can be evaluated by determining the differences in physiological and/or physical condition, including but not limited to, vigor, growth, size, or root length, or specifically, leaf color or leaf area size of the transgenic plant compared to a non-modified plant of the same species grown under the same conditions. Other techniques for evaluating tolerance to abiotic stress include, but are not limited to, measuring chlorophyll fluorescence, photosynthetic rates and gas exchange rates. Further assays for evaluating tolerance to abiotic stress are provided hereinbelow and in the Examples section which follows.

Drought tolerance assay—Soil-based drought screens are performed with plants overexpressing the polynucleotides detailed above. Seeds from control Arabidopsis plants, or other transgenic plants overexpressing nucleic acid of the invention are germinated and transferred to pots. Drought stress is obtained after irrigation is ceased. Transgenic and control plants are compared to each other when the majority of the control plants develop severe wilting. Plants are re-watered after obtaining a significant fraction of the control plants displaying a severe wilting. Plants are ranked comparing to controls for each of two criteria: tolerance to the drought conditions and recovery (survival) following re-watering.

Quantitative parameters of tolerance measured include, but are not limited to, the average wet and dry weight, growth rate, leaf size, leaf coverage (overall leaf area), the weight of the seeds yielded, the average seed size and the number of seeds produced per plant. Transformed plants not exhibiting substantial physiological and/or morphological effects, or exhibiting higher biomass than wild-type plants, are identified as drought stress tolerant plants

Salinity tolerance assay—Transgenic plants with tolerance to high salt concentrations are expected to exhibit better germination, seedling vigor or growth in high salt. Salt stress can be effected in many ways such as, for example, by irrigating the plants with a hyperosmotic solution, by cultivating the plants hydroponically in a hyperosmotic growth solution (e.g., Hoagland solution with added salt), or by culturing the plants in a hyperosmotic growth medium [e.g., 50% Murashige-Skoog medium (MS medium) with added salt]. Since different plants vary considerably in their tolerance to salinity, the salt concentration in the irrigation water, growth solution, or growth medium can be adjusted according to the specific characteristics of the specific plant cultivar or variety, so as to inflict a mild or moderate effect on the physiology and/or morphology of the plants (for guidelines as to appropriate concentration see, Bernstein and Kafkafi, Root Growth Under Salinity Stress In: Plant Roots, The Hidden Half 3rd ed. Waisel Y, Eshel A and Kafkafi U. (editors) Marcel Dekker Inc., New York, 2002, and reference therein).

For example, a salinity tolerance test can be performed by irrigating plants at different developmental stages with increasing concentrations of sodium chloride (for example 50 mM, 150 mM, 300 mM NaCl) applied from the bottom and from above to ensure even dispersal of salt. Following exposure to the stress condition the plants are frequently monitored until substantial physiological and/or morphological effects appear in wild type plants. Thus, the external phenotypic appearance, degree of chlorosis and overall success to reach maturity and yield progeny are compared between control and transgenic plants. Quantitative parameters of tolerance measured include, but are not limited to, the average wet and dry weight, growth rate, leaf size, leaf coverage (overall leaf area), the weight of the seeds yielded, the average seed size and the number of seeds produced per plant. Transformed plants not exhibiting substantial physiological and/or morphological effects, or exhibiting higher biomass than wild-type plants, are identified as abiotic stress tolerant plants.

Osmotic tolerance test—Osmotic stress assays (including sodium chloride and PEG assays) are conducted to determine if an osmotic stress phenotype was sodium chloride-specific or if it was a general osmotic stress related phenotype. Plants which are tolerant to osmotic stress may have more tolerance to drought and/or freezing. For salt and osmotic stress experiments, the medium is supplemented for example with 50 mM, 100 mM, 200 mM NaCl or 15%, 20% or 25% PEG.

Cold stress tolerance—One way to analyze cold stress is as follows. Mature (25 day old) plants are transferred to 4° C. chambers for 1 or 2 weeks, with constitutive light. Later on plants are moved back to greenhouse. Two weeks later damages from chilling period, resulting in growth retardation and other phenotypes, are compared between control and transgenic plants, by measuring plant weight (wet and dry), and by comparing growth rates measured as time to flowering, plant size, yield, and the like.

Heat stress tolerance—One way to measure heat stress tolerance is by exposing the plants to temperatures above 34° C. for a certain period. Plant tolerance is examined after transferring the plants back to 22° C. for recovery and evaluation after 5 days relative to internal controls (non-transgenic plants) or plants not exposed to neither cold or heat stress.

The biomass, vigor and yield of the plant can also be evaluated using any method known to one of ordinary skill in the art. Thus, for example, plant vigor can be calculated by the increase in growth parameters such as leaf area, fiber length, rosette diameter, plant fresh weight and the like per time.

As mentioned, the increase of plant yield can be determined by various parameters. For example, increased yield of rice may be manifested by an increase in one or more of the following: number of plants per growing area, number of panicles per plant, number of spikelets per panicle, number of flowers per panicle, increase in the seed filling rate, increase in thousand kernel weight (1000-weight), increase oil content per seed, increase starch content per seed, among others. An increase in yield may also result in modified architecture, or may occur because of modified architecture. Similarly, increased yield of soybean may be manifested by an increase in one or more of the following: number of plants per growing area, number of pods per plant, number of seeds per pod, increase in the seed filling rate, increase in thousand seed weight (1000-weight), reduce pod shattering, increase oil content per seed, increase protein content per seed, among others. An increase in yield may also result in modified architecture, or may occur because of modified architecture.

Thus, the present invention is of high agricultural value for increasing tolerance of plants to abiotic stress as well as promoting the yield, biomass and vigor of commercially desired crops.

According to another embodiment of the present invention, there is provided a food or feed comprising the plants or a portion thereof of the present invention.

In a further aspect the invention, the transgenic plants of the present invention or parts thereof are comprised in a food or feed product (e.g., dry, liquid, paste). A food or feed product is any ingestible preparation containing the transgenic plants, or parts thereof, of the present invention, or preparations made from these plants. Thus, the plants or preparations are suitable for human (or animal) consumption, i.e. the transgenic plants or parts thereof are more readily digested. Feed products of the present invention further include a oil or a beverage adapted for animal consumption.

It will be appreciated that the transgenic plants, or parts thereof, of the present invention may be used directly as feed products or alternatively may be incorporated or mixed with feed products for consumption. Furthermore, the food or feed products may be processed or used as is. Exemplary feed products comprising the transgenic plants, or parts thereof, include, but are not limited to, grains, cereals, such as oats, e.g. black oats, barley, wheat, rye, sorghum, corn, vegetables, leguminous plants, especially soybeans, root vegetables and cabbage, or green forage, such as grass or hay.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Differential Expression of miRNAs in Maize Plant Under Optimal Versus Abiotic Stress Conditions

Experimental Procedures

Plant Material

Corn seeds were obtained from Galil seeds (Israel). Corn variety GSO308 was used in all experiments. Plants were grown at 24° C. under a 16 hr light: 8 hr dark regime.

Drought Induction

Corn seeds were germinated and grown at 22° C. in soil under normal conditions for 3-4 weeks. Seedlings were then used for experimental assays of each of the following abiotic stresses: drought, salinity and heat shock. For drought induction, irrigation of the stress group was completely stopped for four or six days.

Salt Induction

For salinity induction, irrigation with regular water was substituted by irrigation with 300 mM NaCl solution in the stress group, for overall 2-3 irrigations in a period of four or six days.

Heat Induction

For induction of heat shock, the stress group plants were exposed to a high temperature (37° C.) for one hour.

For all stress analyses, tissue samples from both experimental groups are then used for RNA analysis, as described below.

Total RNA Extraction

Total RNA of leaf or root samples from four to eight biological repeats were extracted using the mirVana™ kit (Ambion, Austin, Tex.) by pooling 3-4 plants to one biological repeat. RNA samples from the two experimental groups of each assay were then loaded onto a microarray for small RNA expression comparison and subsequent identification of differential small RNAs, as described below.

Microarray Design

Custom microarrays were manufactured by Agilent Technologies by in situ synthesis. The first generation microarray consisted of a total of 13619 non-redundant DNA probes, the majority of which arose from deep sequencing data and included different small RNA molecules (i.e. miRNA, siRNA and predicted small RNA sequences), with each probe being printed once. An in-depth analysis of the first generation microarray, which included hybridization experiments as well as structure and orientation verifications on all its small RNAs, resulted in the formation of an improved, second generation, microarray. The second generation microarray consists of a total 4721 non-redundant DNA 45-nucleotide long probes for all known plant small RNAs, with 912 sequences (19.32%) from Sanger version 15 and the rest (3809), encompassing miRNAs (968=20.5%), siRNAs (1626=34.44%) and predicted small RNA sequences (1215=25.74%), from deep sequencing data accumulated by the inventors, with each probe being printed in triplicate. An additional microarray, consisting of 707 sequences from Sanger version 15 was also used in this invention.

Results

Wild type maize plants were allowed to grow at standard, optimal conditions or stress conditions for a period of time as specified above, at the end of which they were evaluated for stress tolerance. Three to four plants from each group were grouped as a biological repeat. Four to eight biological repeats were obtained for each group, and RNA was extracted from leaf or root tissue. The expression level of the maize small RNAs was analyzed by high throughput microarray to identify small RNAs that were differentially expressed between the experimental groups.

Tables 1-5 below present sequences that were found to be differentially expressed in corn grown under drought conditions (lasting four or six days) compared to optimal growth conditions. To clarify, the sequence of an up-regulated miRNA is induced under stress conditions and the sequence of a down-regulated miRNA is repressed under stress conditions.

TABLE 1
Differentially Expressed Small RNAs in Plants Growing
under Drought (4 days) versus Optimal Conditions.

| | Mature | Stem | | |
|---|---|---|---|---|
| | Sequence/ | Loop/ | | |
| | SEQ ID | SEQ ID | | Leaf |
| Mir Name | NO: | NO: | Direction | 4 d |
| Predicted folded 24- | 101 | | up | 2.65 |
| nts-long seq 52214 | | | | |
| Predicted folded 24- | 1 | | down | 2.64 |
| nts-long seq 52255 | | | | |
| Predicted siRNA 55507 | 102 | | up | 4.82 |
| Predicted siRNA 55629 | 103 | | up | 4.97 |
| Predicted siRNA 55869 | 104 | | up | 2.23 |
| Predicted siRNA 55937 | 2 | | down | 2.93 |
| Predicted siRNA 55979 | 105 | | up | 2.58 |
| Predicted siRNA 56759 | 106 | | up | 2.22 |
| Predicted siRNA 57049 | 107 | | up | 2.14 |
| Predicted siRNA 57283 | 108 | | up | 2.52 |
| Predicted siRNA 58170 | 3 | | down | 2.22 |
| Predicted zma mir 47934 | 4 | | down | 2.16 |
| Predicted zma mir 48043 | 109 | | up | 2.04 |
| Predicted zma mir 48120 | 5 | | down | 7.44 |
| Predicted zma mir 48193 | 110 | | up | 3.19 |
| Predicted zma mir 48408 | 6 | | down | 3.58 |
| Predicted zma mir 48451 | 7 | | down | 2.69 |
| Predicted zma mir 48462 | 8 | | down | 3.4 |
| Predicted zma mir 48514 | 111 | | up | 2.2 |
| Predicted zma mir 48520 | 9 | | down | 2.12 |
| Predicted zma mir 48669 | 10 | | down | 2.78 |
| Predicted zma mir 48682 | 11 | | down | 3.11 |
| Predicted zma mir 48841 | 12 | | down | 4.52 |
| Predicted zma mir 48966 | 13 | | down | 3.02 |
| Predicted zma mir 49156 | 14 | | down | 2.08 |
| Predicted zma mir 49199 | 15 | | down | 2.04 |
| Predicted zma mir 50109 | 112 | | up | 2.18 |
| Predicted zma mir 50425 | 113 | | up | 2.31 |
| tae-miR1125 | 114 | 217 | up | 2.07 |

TABLE 2
Differentially Expressed Small RNAs in Plants Growing
under Drought (6 days) versus Optimal Conditions.

| | Mature | | | | Fold | Fold |
|---|---|---|---|---|---|---|
| | Sequence/ | Stem Loop/ | old | | Change | Change |
| Mir Name | SEQ ID NO: | SEQ ID NO: | numbers | Direction | Leaf 6 d | Root 6 d |
| ath-miR164c | 115 | 218 | 218 | up | 1.94 | 1.51 |
| osa- | 116 | 219 | 219 | up | 3.69 | |
| miR2907a | | | | | | |
| Predicted | 117 | | | up | 3.94 | |
| folded 24- | | | | | | |
| nts-long seq | | | | | | |
| 52214 | | | | | | |
| Predicted | 16 | | | down | 2.95 | |
| folded 24- | | | | | | |
| nts-long seq | | | | | | |
| 52255 | | | | | | |
| Predicted | 118 | | | up | 2.09 | |
| folded 24- | | | | | | |
| nts-long seq | | | | | | |
| 52285 | | | | | | |
| Predicted | 119 | | | up | 2.99 | |
| folded 24- | | | | | | |
| nts-long seq | | | | | | |
| 52953 | | | | | | |
| Predicted | 120 | | | up | 4.24 | |
| folded 24- | | | | | | |
| nts-long seq | | | | | | |
| 53693 | | | | | | |
| Predicted | 121 | | | up | 9.27 | |
| siRNA 55507 | | | | | | |
| Predicted | 122 | | | up | 8.68 | |
| siRNA 55629 | | | | | | |
| Predicted | 123 | | | up | 2.93 | |
| siRNA 55775 | | | | | | |
| Predicted | 124 | | | up | 3.08 | |
| siRNA 55869 | | | | | | |
| Predicted | 17 | | | down | 2.82 | |
| siRNA 55937 | | | | | | |
| Predicted | 18 | | | down | 2.86 | |
| siRNA 56066 | | | | | | |
| Predicted | 125 | | | up | 2.69 | |
| siRNA 56759 | | | | | | |
| Predicted | 126 | | | up | 2.67 | |
| siRNA 57049 | | | | | | |
| Predicted | 127 | | | up | 3.91 | |
| siRNA 57283 | | | | | | |
| Predicted | 19 | | | down | 5.99 | |
| siRNA 58170 | | | | | | |
| Predicted | 128 | | | up | 2.73 | |
| siRNA 58574 | | | | | | |
| Predicted | 129 | | | up | 4.18 | |
| siRNA 60433 | | | | | | |
| Predicted | 20 | | | down | 2.47 | |
| zma mir | | | | | | |
| 47934 | | | | | | |
| Predicted | 130 | | | up | 2.4 | |
| zma mir | | | | | | |
| 48043 | | | | | | |
| Predicted | 21 | | | down | 9.3 | |
| zma mir | | | | | | |
| 48120 | | | | | | |
| Predicted | 131 | | | up | 5.34 | |
| zma mir | | | | | | |
| 48193 | | | | | | |
| Predicted | 22 | | | down | 3.23 | |
| zma mir | | | | | | |
| 48408 | | | | | | |
| Predicted | 23 | | | down | 2.69 | |
| zma mir | | | | | | |
| 48451 | | | | | | |
| Predicted | 24 | | | down | 5.49 | |
| zma mir | | | | | | |
| 48462 | | | | | | |
| Predicted | 25 | | | down | 6.19 | |
| zma mir | | | | | | |
| 48520 | | | | | | |
| Predicted | 26 | | | down | 2.98 | |
| zma mir | | | | | | |
| 48653 | | | | | | |
| Predicted | 27 | | | down | 2.33 | |
| zma mir | | | | | | |
| 48669 | | | | | | |
| Predicted | 28 | | | down | 2.86 | |
| zma mir | | | | | | |
| 48682 | | | | | | |
| Predicted | 29 | | | down | 5.01 | |
| zma mir | | | | | | |
| 48841 | | | | | | |
| Predicted | 30 | | | down | 2.54 | |
| zma mir | | | | | | |
| 48966 | | | | | | |
| Predicted | 31 | | | down | 4.39 | |
| zma mir | | | | | | |
| 49156 | | | | | | |
| Predicted | 32 | | | down | 2.8 | |
| zma mir | | | | | | |
| 49199 | | | | | | |
| sbi-miR164c | 132 | 220 | 220 | up | 1.86 | |
| tae-miR1125 | 133 | 222 | 221 | up | 2.58 | |

TABLE 3
Differentially Expressed Small RNAs in Plants Growing
under High Salt (4 days) versus Optimal Conditions

| | | | Fold |
|---|---|---|---|
| | | | Change |
| | SEQ ID | | Leaf |
| Name Mir | NO: | Direction | 4 d |
| folded 24-nts-long | 33 | down | 2.34 |
| Predicted seq 54187 | | | |
| siRNA 54673 Predicted | 134 | up | 2.08 |
| siRNA 54895 Predicted | 135 | up | 2.17 |
| siRNA 55242 Predicted | 136 | up | 2.09 |
| siRNA 55246 Predicted | 137 | up | 2.64 |
| siRNA 55344 Predicted | 138 | up | 2.35 |
| siRNA 55402 Predicted | 139 | up | 2.46 |
| siRNA 55909 Predicted | 140 | up | 2.06 |
| siRNA 56060 Predicted | 141 | up | 2.31 |
| siRNA 56305 Predicted | 142 | up | 2.43 |
| siRNA 56314 Predicted | 143 | up | 2.31 |
| siRNA 56506 Predicted | 144 | up | 2.3 |
| siRNA 56651 Predicted | 145 | up | 2.42 |
| siRNA 57169 Predicted | 146 | up | 2.25 |
| siRNA 57197 Predicted | 147 | up | 2.15 |
| siRNA 58212 Predicted | 148 | up | 2.66 |
| siRNA 59035 Predicted | 149 | up | 6.12 |
| siRNA 59453 Predicted | 150 | up | 2.73 |
| zma mir 47990 Predicted | 34 | down | 2.69 |
| zma mir 48459 Predicted | 45 (35) | down | 2.37 |
| zma mir 48490 Predicted | 151 | up | 3.12 |
| zma mir 48753 Predicted | 36 | down | 2.11 |
| zma mir 48783 Predicted | 152 | up | 2.35 |
| zma mir 48824 Predicted | 37 | down | 2.08 |
| zma mir 48848 Predicted | 38 | down | 2.06 |
| zma mir 49575 Predicted | 39 | down | 2.49 |
| zma mir 49817 Predicted | 40 | down | 2.8 |
| zma mir 49855 Predicted | 41 | down | 2.69 |
| zma mir 49862 Predicted | 52 (42) | down | 2.16 |
| zma mir 50145 Predicted | 153 | up | 3.92 |

TABLE 4
Differentially Expressed Small RNAs in Plants Growing
under High Salt (6 days) versus Optimal Conditions

| | | | Fold |
|---|---|---|---|
| | | | Change |
| | SEQ ID | | Leaf |
| Mir Name | NO: | Direction | 6 d |
| Predicted folded 24- | 43 | down | 2.2 |
| nts-long seq 54187 | | | |
| Predicted siRNA 54673 | 154 | up | 5.57 |
| Predicted siRNA 54895 | 155 | up | 6.11 |
| Predicted siRNA 55242 | 156 | up | 5.91 |
| Predicted siRNA 55246 | 157 | up | 6.3 |
| Predicted siRNA 55344 | 158 | up | 8.56 |
| Predicted siRNA 55402 | 159 | up | 4.96 |
| Predicted siRNA 55909 | 160 | up | 2.58 |
| Predicted siRNA 56060 | 161 | up | 2.95 |
| Predicted siRNA 56305 | 162 | up | 4.66 |
| Predicted siRNA 56314 | 163 | up | 5.97 |
| Predicted siRNA 56506 | 164 | up | 5.98 |
| Predicted siRNA 56651 | 165 | up | 5.5 |
| Predicted siRNA 57169 | 166 | up | 5.52 |
| Predicted siRNA 57197 | 167 | up | 3.98 |
| Predicted siRNA 58212 | 168 | up | 3.29 |
| Predicted siRNA 59035 | 169 | up | 8.47 |
| Predicted siRNA 59453 | 170 | up | 2.12 |
| Predicted zma mir 47990 | 44 | down | 4.22 |
| Predicted zma mir 48459 | 45 (35) | down | 3.21 |
| Predicted zma mir 48490 | 171 | up | 2.71 |
| Predicted zma mir 48753 | 46 | down | 3.3 |
| Predicted zma mir 48783 | 172 | up | 2.69 |
| Predicted zma mir 48824 | 47 | down | 2.17 |
| Predicted zma mir 48848 | 48 | down | 2.38 |
| Predicted zma mir 49575 | 49 | down | 2.07 |
| Predicted zma mir 49817 | 50 | down | 3.26 |
| Predicted zma mir 49855 | 51 | down | 3.01 |
| Predicted zma mir 49862 | 52 (42) | down | 2.88 |
| Predicted zma mir 50145 | 173 | up | 3.66 |

TABLE 5
Differentially Expressed Small RNAs in Plants Growing
under Heat Shock (1 hour) versus Optimal Conditions

| | | | Fold |
|---|---|---|---|
| | | | Change |
| | SEQ ID | | Leaf |
| Mir Name | NO: | Direction | 1 hour |
| Predicted folded 24- | 174 | up | 2.7 |
| nts-long seq 50957 | | | |
| Predicted folded 24- | 175 | up | 3.56 |
| nts-long seq 51391 | | | |
| Predicted folded 24- | 176 | up | 2.47 |
| nts-long seq 51709 | | | |
| Predicted folded 24- | 177 | up | 2.46 |
| nts-long seq 52606 | | | |
| Predicted folded 24- | 178 | up | 2.47 |
| nts-long seq 52682 | | | |
| Predicted folded 24- | 53 | down | 2.25 |
| nts-long seq 52724 | | | |
| Predicted folded 24- | 179 | up | 4.42 |
| nts-long seq 53851 | | | |
| Predicted folded 24- | 54 | down | 4.51 |
| nts-long seq 53866 | | | |
| Predicted siRNA 54548 | 180 | up | 3.72 |
| Predicted siRNA 54566 | 181 | up | 4.34 |
| Predicted siRNA 54666 | 182 | up | 2.42 |
| Predicted siRNA 54735 | 55 | down | 4.98 |
| Predicted siRNA 55208 | 56 | down | 3.8 |
| Predicted siRNA 55684 | 183 | up | 3.16 |
| Predicted siRNA 55793 | 184 | up | 2.62 |
| Predicted siRNA 55824 | 185 | up | 6.85 |
| Predicted siRNA 55968 | 186 | up | 2.35 |
| Predicted siRNA 56154 | 187 | up | 2.03 |
| Predicted siRNA 56225 | 188 | up | 3.61 |
| Predicted siRNA 56396 | 57 | down | 3.08 |
| Predicted siRNA 56582 | 189 | up | 2.51 |
| Predicted siRNA 56658 | 190 | up | 2.19 |
| Predicted siRNA 56664 | 191 | up | 3.07 |
| Predicted siRNA 56791 | 58 | down | 4.75 |
| Predicted siRNA 56885 | 192 | up | 2.24 |
| Predicted siRNA 57061 | 193 | up | 3.15 |
| Predicted siRNA 57689 | 59 | down | 2.69 |
| Predicted siRNA 58105 | 194 | up | 2.55 |
| Predicted siRNA 58108 | 60 | down | 6.63 |
| Predicted siRNA 58158 | 61 | down | 3.98 |
| Predicted siRNA 58387 | 195 | up | 2.77 |
| Predicted siRNA 58717 | 196 | up | 3.46 |
| Predicted siRNA 58720 | 62 | down | 3.54 |
| Predicted siRNA 58740 | 63 | down | 2.88 |
| Predicted siRNA 59056 | 64 | down | 2.29 |
| Predicted siRNA 59211 | 65 | down | 5.31 |
| Predicted siRNA 59300 | 66 | down | 3.04 |
| Predicted siRNA 59379 | 67 | down | 2.32 |
| Predicted siRNA 59410 | 68 | down | 6.53 |
| Predicted siRNA 59474 | 69 | down | 6.01 |
| Predicted siRNA 59580 | 197 | up | 3.95 |
| Predicted siRNA 59736 | 70 | down | 3.34 |
| Predicted siRNA 59799 | 71 | down | 2.22 |
| Predicted siRNA 59800 | 72 | down | 2 |
| Predicted siRNA 59817 | 198 | up | 7.86 |
| Predicted siRNA 59820 | 73 | down | 3.79 |
| Predicted siRNA 59851 | 74 | down | 9.21 |
| Predicted siRNA 59918 | 75 | down | 9.26 |
| Predicted siRNA 59935 | 76 | down | 2.06 |
| Predicted siRNA 59937 | 77 | down | 2.61 |
| Predicted siRNA 59987 | 199 | up | 12.68 |
| Predicted siRNA 60036 | 200 | up | 2.16 |
| Predicted siRNA 60421 | 78 | down | 6.25 |
| Predicted siRNA 60533 | 79 | down | 6.03 |
| Predicted siRNA 60635 | 201 | up | 3.01 |
| Predicted siRNA 60718 | 202 | up | 2.18 |
| Predicted siRNA 60742 | 203 | up | 2.07 |
| Predicted siRNA 60833 | 80 | down | 2.45 |
| Predicted siRNA 60993 | 204 | up | 2.37 |
| Predicted siRNA 61212 | 81 | down | 2.14 |
| Predicted siRNA 61236 | 82 | down | 2.27 |
| Predicted zma mir 47966 | 205 | up | 3.04 |
| Predicted zma mir 48327 | 83 | down | 2.04 |
| Predicted zma mir 48479 | 206 | up | 3.92 |
| Predicted zma mir 48482 | 207 | up | 2.91 |
| Predicted zma mir 48489 | 84 | down | 2.98 |
| Predicted zma mir 48790 | 208 | up | 2.04 |
| Predicted zma mir 48905 | 209 | up | 3.72 |
| Predicted zma mir 49248 | 85 | down | 4.94 |
| Predicted zma mir 49259 | 210 | up | 3.77 |
| Predicted zma mir 49310 | 86 | down | 4.45 |
| Predicted zma mir 49642 | 211 | up | 4.3 |
| Predicted zma mir 49718 | 212 | up | 3.57 |
| Predicted zma mir 49952 | 87 | down | 2.41 |
| Predicted zma mir 50085 | 213 | up | 2.43 |
| Predicted zma mir 50120 | 88 | down | 2.92 |
| Predicted zma mir 50166 | 89 | down | 2.9 |
| Predicted zma mir 50256 | 90 | down | 6.25 |
| Predicted zma mir 50289 | 91 | down | 2.01 |
| Predicted zma mir 50388 | 92 | down | 2.43 |
| Predicted zma mir 50449 | 214 | up | 2.25 |
| Predicted zma mir 50453 | 93 | down | 7.88 |
| Predicted zma mir 50480 | 94 | down | 2.21 |
| Predicted zma mir 50481 | 95 | down | 2.3 |
| Predicted zma mir 50483 | 96 | down | 2.23 |
| Predicted zma mir 50486 | 215 | up | 2.34 |
| Predicted zma mir 50522 | 216 | up | 2.73 |
| Predicted zma mir 50570 | 97 | down | 2.07 |
| Predicted zma mir 50682 | 98 | down | 2.21 |
| Predicted zma mir 50695 | 99 | down | 2.02 |
| Predicted zma mir 50701 | 100 | down | 3.38 |

### Example 2

### Identification of Homologous and Orthologous Sequences of Differential Small RNAs Associated with Enhanced Abiotic Stress Tolerance

The small RNA sequences of the invention that were either down- or up-regulated under abiotic stress conditions were examined for homologous and orthologous sequences using the miRBase database (www.mirbase.org/) and the Plant MicroRNA Database (PMRD, http://bioinformatics.cau.edu.cn/PMRD). The mature miRNA sequences that are homologous or orthologous to the miRNAs of the invention (listed in Tables 1-5 above) are found using miRNA public databases, having at least 75% identity of the mature small RNA, and are summarized in Tables 6-8 below.

TABLE 6
Homologs of Small RNAs Listed in Tables 1-2 Above
(Differentially Expressed Under Drought Stress)

| | | | | | | Homolog | |
|---|---|---|---|---|---|---|---|
| | Mature | | | | | Stem-loop | |
| Mir | Sequence/ | Mir | Homologs | Sequence/ | Homolog | Sequence/ | |
| Name | SEQ ID NO: | Length | Names | SEQ ID NO: | Length | SEQ ID NO: | % Identity |
| Predicted | 133 | 21 | aly- | 223 | 21 | 417 | 0.95 |
| zma mir | | | miR168a* | | | | |
| 50425 | | | | | | | |
| | | | hvu- | 224 | 24 | 418 | 0.95 |
| | | | miR168-3p | | | | |
| | | | osa- | 225 | 24 | 419 | 0.95 |
| | | | miR168a- | | | | |
| | | | 3p | | | | |
| | | | zma- | 226 | 20 | 420 | 0.9 |
| | | | miR168a* | | | | |
| | | | zma- | 227 | 20 | 421 | 0.95 |
| | | | miR168b* | | | | |
| osa- | 116 | 22 | osa- | 264 | 22 | 458 | 1 |
| miR2907a | | | miR2907b | | | | |
| | | | osa- | 265 | 22 | 459 | 1 |
| | | | miR2907c | | | | |
| | | | osa- | 266 | 22 | 460 | 1 |
| | | | miR2907d | | | | |
| ath- | 115 | 21 | aly- | 267 | 21 | 461 | 0.95 |
| miR164c | | | miR164a | | | | |
| | | | aly- | 268 | 21 | 462 | 0.95 |
| | | | miR164b | | | | |
| | | | aly- | 269 | 21 | 463 | 1 |
| | | | miR164c | | | | |
| | | | ath- | 270 | 21 | 464 | 0.95 |
| | | | miR164a | | | | |
| | | | ath- | 271 | 21 | 465 | 0.95 |
| | | | miR164b | | | | |
| | | | bdi- | 272 | 21 | 466 | 0.95 |
| | | | miR164a | | | | |
| | | | bdi- | 273 | 21 | 467 | 0.95 |
| | | | miR164b | | | | |
| | | | bdi- | 274 | 21 | 468 | 0.95 |
| | | | miR164c | | | | |
| | | | bdi- | 275 | 21 | 469 | 0.95 |
| | | | miR164d | | | | |
| | | | bdi- | 276 | 21 | 470 | 0.95 |
| | | | miR164e | | | | |
| | | | bdi- | 277 | 21 | 471 | 0.86 |
| | | | miR164f | | | | |
| | | | bna- | 278 | 21 | 472 | 0.95 |
| | | | miR164 | | | | |
| | | | bra- | 279 | 21 | 473 | 0.95 |
| | | | miR164a | | | | |
| | | | cav- | 280 | 21 | 474 | 0.95 |
| | | | miR164 | | | | |
| | | | csi-miR164 | 281 | 21 | 475/605 | 0.95 |
| | | | ctr-miR164 | 282 | 21 | 476 | 0.95 |
| | | | far- | 283 | 21 | 477 | 0.9 |
| | | | miR164a | | | | |
| | | | far- | 284 | 21 | 478 | 0.9 |
| | | | miR164b | | | | |
| | | | gar- | 285 | 21 | 479 | 0.86 |
| | | | miR164 | | | | |
| | | | ghr- | 286 | 21 | 480 | 0.95 |
| | | | miR164 | | | | |
| | | | gma- | 287 | 21 | 481 | 0.95 |
| | | | miR164 | | | | |
| | | | ini-miR164 | 288 | 21 | 482 | 0.9 |
| | | | mtr- | 289 | 21 | 483 | 0.95 |
| | | | miR164a | | | | |
| | | | mtr- | 290 | 21 | 484 | 0.95 |
| | | | miR164b | | | | |
| | | | mtr- | 291 | 21 | 485 | 0.95 |
| | | | miR164c | | | | |
| | | | mtr- | 292 | 21 | 486 | 0.9 |
| | | | miR164d | | | | |
| | | | osa- | 293 | 21 | 487 | 0.95 |
| | | | miR164a | | | | |
| | | | osa- | 294 | 21 | 488 | 0.95 |
| | | | miR164b | | | | |
| | | | osa- | 295 | 21 | 489 | 0.9 |
| | | | miR164c | | | | |
| | | | osa- | 296 | 21 | 490 | 0.95 |
| | | | miR164d | | | | |
| | | | osa- | 297 | 21 | 491 | 0.95 |
| | | | miR164e | | | | |
| | | | osa- | 298 | 21 | 492 | 0.95 |
| | | | miR164f | | | | |
| | | | ppl- | 299 | 21 | 493 | 0.95 |
| | | | miR164 | | | | |
| | | | ptc- | 300 | 21 | 494 | 0.95 |
| | | | miR164a | | | | |
| | | | ptc- | 301 | 21 | 495 | 0.95 |
| | | | miR164b | | | | |
| | | | ptc- | 302 | 21 | 496 | 0.95 |
| | | | miR164c | | | | |
| | | | ptc- | 303 | 21 | 497 | 0.95 |
| | | | miR164d | | | | |
| | | | ptc- | 304 | 21 | 498 | 0.95 |
| | | | miR164e | | | | |
| | | | ptc- | 305 | 21 | 499 | 0.9 |
| | | | miR164f | | | | |
| | | | rco- | 306 | 21 | 500 | 0.95 |
| | | | miR164a | | | | |
| | | | rco- | 307 | 21 | 501 | 0.95 |
| | | | miR164b | | | | |
| | | | rco- | 308 | 21 | 502 | 0.95 |
| | | | miR164c | | | | |
| | | | rco- | 309 | 21 | 503 | 0.9 |
| | | | miR164d | | | | |
| | | | sbi- | 310 | 21 | 504 | 0.95 |
| | | | miR164 | | | | |
| | | | sbi- | 311 | 21 | 505 | 0.95 |
| | | | miR164b | | | | |
| | | | sbi- | 312 | 21 | 506 | 0.9 |
| | | | miR164c | | | | |
| | | | sbi- | 313 | 21 | 507 | 0.95 |
| | | | miR164d | | | | |
| | | | sbi- | 314 | 21 | 508 | 0.95 |
| | | | miR164e | | | | |
| | | | tae- | 315 | 21 | 509 | 0.95 |
| | | | miR164 | | | | |
| | | | tae- | 316 | 21 | 510 | 0.95 |
| | | | miR164b | | | | |
| | | | tcc- | 317 | 21 | 511 | 0.95 |
| | | | miR164a | | | | |
| | | | tcc- | 318 | 21 | 512 | 0.95 |
| | | | miR164b | | | | |
| | | | tcc- | 319 | 21 | 513 | 0.9 |
| | | | miR164c | | | | |
| | | | vvi- | 320 | 21 | 514 | 0.95 |
| | | | miR164a | | | | |
| | | | vvi- | 321 | 21 | 515 | 0.9 |
| | | | miR164b | | | | |
| | | | vvi- | 322 | 21 | 516 | 0.95 |
| | | | miR164c | | | | |
| | | | vvi- | 323 | 21 | 517 | 0.95 |
| | | | miR164d | | | | |
| | | | zma- | 324 | 21 | 518 | 0.95 |
| | | | miR164a | | | | |
| | | | zma- | 325 | 21 | 519 | 0.95 |
| | | | miR164b | | | | |
| | | | zma- | 326 | 21 | 520 | 0.95 |
| | | | miR164c | | | | |
| | | | zma- | 327 | 21 | 521 | 0.95 |
| | | | miR164d | | | | |
| | | | zma- | 411 | 21 | 522/606 | 0.90/0.95 |
| | | | miR164e | | | | |
| | | | zma- | 412 | 21 | 523/607 | 0.95 |
| | | | miR164f | | | | |
| | | | zma- | 330 | 21 | 524/608 | 0.95 |
| | | | miR164g | | | | |
| | | | zma- | 413 | 21 | 525/609 | 0.95 |
| | | | miR164h | | | | |
| sbi- | 132 | 21 | aly- | 332 | 21 | 526 | 0.86 |
| miR164c | | | miR164a | | | | |
| | | | aly- | 333 | 21 | 527 | 0.86 |
| | | | miR164b | | | | |
| | | | aly- | 334 | 21 | 528 | 0.9 |
| | | | miR164c | | | | |
| | | | ath- | 335 | 21 | 529 | 0.86 |
| | | | miR164a | | | | |
| | | | ath- | 336 | 21 | 530 | 0.86 |
| | | | miR164b | | | | |
| | | | ath- | 337 | 21 | 531 | 0.9 |
| | | | miR164c | | | | |
| | | | bdi- | 338 | 21 | 532 | 0.86 |
| | | | miR164a | | | | |
| | | | bdi- | 339 | 21 | 533 | 0.86 |
| | | | miR164b | | | | |
| | | | bdi- | 340 | 21 | 534 | 0.86 |
| | | | miR164c | | | | |
| | | | bdi- | 341 | 21 | 535 | 0.86 |
| | | | miR164d | | | | |
| | | | bdi- | 342 | 21 | 536 | 0.86 |
| | | | miR164e | | | | |
| | | | bdi- | 343 | 21 | 537 | 0.76 |
| | | | miR164f | | | | |
| | | | bna- | 344 | 21 | 538 | 0.86 |
| | | | miR164 | | | | |
| | | | bra- | 345 | 21 | 539 | 0.86 |
| | | | miR164a | | | | |
| | | | cav- | 346 | 21 | 540 | 0.86 |
| | | | miR164 | | | | |
| | | | csi-miR164 | 347 | 21 | 541/611 | 0.86 |
| | | | ctr-miR164 | 348 | 21 | 542 | 0.86 |
| | | | far- | 349 | 21 | 543 | 0.81 |
| | | | miR164a | | | | |
| | | | far- | 350 | 21 | 544 | 0.81 |
| | | | miR164b | | | | |
| | | | gar- | 351 | 21 | 545 | 0.86 |
| | | | miR164 | | | | |
| | | | ghr- | 352 | 21 | 546 | 0.86 |
| | | | miR164 | | | | |
| | | | gma- | 353 | 21 | 547 | 0.86 |
| | | | miR164 | | | | |
| | | | ini-miR164 | 354 | 21 | 548 | 0.86 |
| | | | mtr- | 355 | 21 | 549 | 0.86 |
| | | | miR164a | | | | |
| | | | mtr- | 356 | 21 | 550 | 0.86 |
| | | | miR164b | | | | |
| | | | mtr- | 357 | 21 | 551 | 0.86 |
| | | | miR164c | | | | |
| | | | mtr- | 358 | 21 | 552 | 0.81 |
| | | | miR164d | | | | |
| | | | osa- | 359 | 21 | 553 | 0.86 |
| | | | miR164a | | | | |
| | | | osa- | 360 | 21 | 554 | 0.86 |
| | | | miR164b | | | | |
| | | | osa- | 361 | 21 | 555 | 0.81 |
| | | | miR164c | | | | |
| | | | osa- | 362 | 21 | 556 | 0.86 |
| | | | miR164d | | | | |
| | | | osa- | 363 | 21 | 557 | 0.95 |
| | | | miR164e | | | | |
| | | | osa- | 364 | 21 | 558 | 0.86 |
| | | | miR164f | | | | |
| | | | ppl- | 365 | 21 | 559 | 0.86 |
| | | | miR164 | | | | |
| | | | ptc- | 366 | 21 | 560 | 0.86 |
| | | | miR164a | | | | |
| | | | ptc- | 367 | 21 | 561 | 0.86 |
| | | | miR164b | | | | |
| | | | ptc- | 368 | 21 | 562 | 0.86 |
| | | | miR164c | | | | |
| | | | ptc- | 369 | 21 | 563 | 0.86 |
| | | | miR164d | | | | |
| | | | ptc- | 370 | 21 | 564 | 0.86 |
| | | | miR164e | | | | |
| | | | ptc- | 371 | 21 | 565 | 0.81 |
| | | | miR164f | | | | |
| | | | rco- | 372 | 21 | 566 | 0.86 |
| | | | miR164a | | | | |
| | | | rco- | 373 | 21 | 567 | 0.86 |
| | | | miR164b | | | | |
| | | | rco- | 374 | 21 | 568 | 0.86 |
| | | | miR164c | | | | |
| | | | rco- | 375 | 21 | 569 | 0.81 |
| | | | miR164d | | | | |
| | | | sbi- | 376 | 21 | 570 | 0.86 |
| | | | miR164 | | | | |
| | | | sbi- | 377 | 21 | 571 | 0.86 |
| | | | miR164b | | | | |
| | | | sbi- | 378 | 21 | 572 | 0.86 |
| | | | miR164d | | | | |
| | | | sbi- | 379 | 21 | 573 | 0.86 |
| | | | miR164e | | | | |
| | | | tae- | 380 | 21 | 574 | 0.86 |
| | | | miR164 | | | | |
| | | | tae- | 381 | 21 | 575 | 0.86 |
| | | | miR164b | | | | |
| | | | tcc- | 382 | 21 | 576 | 0.86 |
| | | | miR164a | | | | |
| | | | tcc- | 383 | 21 | 577 | 0.86 |
| | | | miR164b | | | | |
| | | | tcc- | 384 | 21 | 578 | 0.81 |
| | | | miR164c | | | | |
| | | | vvi- | 385 | 21 | 579 | 0.86 |
| | | | miR164a | | | | |
| | | | vvi- | 386 | 21 | 580 | 0.81 |
| | | | miR164b | | | | |
| | | | vvi- | 387 | 21 | 581 | 0.86 |
| | | | miR164c | | | | |
| | | | vvi- | 388 | 21 | 582 | 0.86 |
| | | | miR164d | | | | |
| | | | zma- | 389 | 21 | 583 | 0.86 |
| | | | miR164a | | | | |
| | | | zma- | 390 | 21 | 584 | 0.86 |
| | | | miR164b | | | | |
| | | | zma- | 391 | 21 | 585 | 0.86 |
| | | | miR164c | | | | |
| | | | zma- | 392 | 21 | 586 | 0.86 |
| | | | miR164d | | | | |
| | | | zma- | 414 | 21 | 587/611 | 1.00/0.86 |
| | | | miR164e | | | | |
| | | | zma- | 415 | 21 | 588/612 | 0.86 |
| | | | miR164f | | | | |
| | | | zma- | 395 | 21 | 589/613 | 0.86 |
| | | | miR164g | | | | |
| | | | zma- | 416 | 21 | 590/614 | 0.90/0.86 |
| | | | miR164h | | | | |
| Predicted | 112 | 21 | aly- | 397 | 21 | 591 | 0.9 |
| zma mir | | | miR827 | | | | |
| 50109 | | | | | | | |
| | | | ath- | 398 | 21 | 592 | 0.9 |
| | | | miR827 | | | | |
| | | | bdi- | 399 | 21 | 593 | 1 |
| | | | miR827 | | | | |
| | | | csi- | 400 | 21 | 594 | 0.95 |
| | | | miR827 | | | | |
| | | | ghr- | 401 | 21 | 595 | 0.95 |
| | | | miR827a | | | | |
| | | | ghr- | 402 | 21 | 596 | 0.95 |
| | | | miR827b | | | | |
| | | | ghr- | 403 | 21 | 597 | 0.95 |
| | | | miR827c | | | | |
| | | | osa- | 404 | 21 | 598 | 0.86 |
| | | | miR827 | | | | |
| | | | osa- | 405 | 21 | 599 | 1 |
| | | | miR827a | | | | |
| | | | osa- | 406 | 21 | 600 | 1 |
| | | | miR827b | | | | |
| | | | ptc- | 407 | 21 | 601 | 0.86 |
| | | | miR827 | | | | |
| | | | ssp- | 408 | 21 | 602 | 1 |
| | | | miR827 | | | | |
| | | | tcc- | 409 | 21 | 603 | 0.95 |
| | | | miR827 | | | | |
| | | | zma- | 410 | 21 | 604 | 1 |
| | | | miR827 | | | | |

TABLE 7
Homologs of Small RNAs Listed in Tables 3-4 Above
(Differentially Expressed Under Salinity Stress)

| | Mature | | | Homolog | | Stem Loop | |
|---|---|---|---|---|---|---|---|
| Mir | Sequence/ | Mir | Homologs | Sequence/ | Homolog | Sequence/ | |
| Name | SEQ ID NO: | Length | Names | SEQ ID NO: | Length | SEQ ID NO: | % Identity |
| Predicted | 52 | 21 | bdi- | 615 | 21 | 627 | 0.9 |
| zma mir | | | miR528 | | | | |
| 49862 | | | | | | | |
| | | | osa- | 616 | 21 | 628 | 0.9 |
| | | | miR528 | | | | |
| | | | sbi- | 617 | 21 | 629 | 0.9 |
| | | | miR528 | | | | |
| | | | ssp- | 618 | 21 | 630 | 0.9 |
| | | | miR528 | | | | |
| | | | zma- | 619 | 21 | 631 | 0.9 |
| | | | miR528a | | | | |
| | | | zma- | 620 | 21 | 632 | 0.9 |
| | | | miR528b | | | | |
| Predicted | 45 | 21 | bdi- | 621 | 21 | 633 | 0.9 |
| zma mir | | | miR528 | | | | |
| 48459 | | | | | | | |
| | | | osa- | 622 | 21 | 634 | 0.9 |
| | | | miR528 | | | | |
| | | | sbi- | 623 | 21 | 635 | 0.9 |
| | | | miR528 | | | | |
| | | | ssp- | 624 | 21 | 636 | 0.9 |
| | | | miR528 | | | | |
| | | | zma- | 625 | 21 | 637 | 0.9 |
| | | | miR528a | | | | |
| | | | zma- | 626 | 21 | 638 | 0.9 |
| | | | miR528b | | | | |

TABLE 8
Homologs of Small RNAs Listed in Table 5 Above (Differentially
Expressed Under Heat Shock Stress)

| | | | | | | Stem Loop | |
|---|---|---|---|---|---|---|---|
| Mir | Mature | Mir | Homologs | Sequence/ | Homolog | Sequence/ | |
| Name | Sequence | Length | Names | SEQ ID NO: | Length | SEQ ID NO: | % Identity |
| Predicted | 85 | 21 | zma- | 639 | 21 | 640 | 0.9 |
| zma mir | | | miR398b* | | | | |
| 49248 | | | | | | | |

### Example 3

### Verification of Expression of Small RNA Molecules Associated with Abiotic Stress

Small RNAs that are potentially associated with improved abiotic or biotic stress tolerance are first identified by proprietary computational algorithms that analyze RNA expression profiles alongside publicly available gene and protein databases. A high throughput screening is performed on microarrays loaded with miRNAs that were found to be differential under multiple stress and optimal environmental conditions and in different plant tissues. Following identification of small RNA molecules potentially involved in maize abiotic stress tolerance using bioinformatics tools, the actual mRNA levels in an experiment are determined using reverse transcription assay followed by quantitative Real-Time PCR (qRT-PCR) analysis. RNA levels are compared between different tissues, developmental stages, growing conditions and/or genetic backgrounds incorporated in each experiment. A correlation analysis between mRNA levels in different experimental conditions/genetic backgrounds is applied and used as evidence for the role of the gene in the plant.

Experimental Procedures

Root and leaf samples are freshly excised from maize plants grown as described above on Murashige-Skoog (Duchefa). Experimental plants are grown either under optimal irrigation conditions, salt levels or temperatures to be used as a control group, or under stressful conditions of prolonged water deprivation, high salt concentrations and a heat shock treatment at a temperature higher than 34° C. to be used as stress-induced groups to assess the drought, salinity and heat shock tolerance, respectively, of control versus transgenic plants. Total RNA is extracted from the different tissues, using mirVana™ commercial kit (Ambion) following the protocol provided by the manufacturer. For measurement and verification of messenger RNA (mRNA) expression level of all genes, reverse transcription followed by quantitative real time PCR (qRT-PCR) is performed on total RNA extracted from each plant tissue (i.e., roots and leaves) from each experimental group as described above. To elaborate, reverse transcription is performed on 1 μg total RNA, using a miScript Reverse Transcriptase kit (Qiagen), following the protocol suggested by the manufacturer. Quantitative RT-PCR is performed on cDNA (0.1 ng/μl final concentration), using a miScript SYBR GREEN PCR (Qiagen) forward (based on the miR sequence itself) and reverse primers (supplied with the kit). All qRT-PCR reactions are performed in triplicates using an ABI7500 real-time PCR machine, following the recommended protocol for the machine. To normalize the expression level of miRNAs associated with enhanced NUE between the different tissues and growing conditions of the maize plants, normalizer miRNAs are selected and used for comparison. Normalizer miRNAs, which are miRNAs with unchanged expression level between tissues and growing conditions, are custom selected for each experiment. The normalization procedure consists of second-degree polynomial fitting to a reference data (which is the median vector of all the data—excluding outliers) as described by Rosenfeld et al (2008, Nat Biotechnol, 26(4):462-469). A summary of primers for the differential small RNA molecules that will be used in the qRT-PCR validation and analysis is presented in Tables 9-11 below.

TABLE 9
Primers for qRT-PCR Analysis of small RNAs
Differentially Expressed in Drought

| | Primer Sequence/ | Primer | |
|---|---|---|---|
| miR Name | SEQ ID NO: | length | Tm |
| ath-miR164c | 641 | 21 | 64 |
| osa-miR2907a | 642 | 20 | 67 |
| Predicted folded 24-nts-long seq | 643 | 24 | 66 |
| 52214 | | | |
| Predicted folded 24-nts-long seq | 644 | 24 | 59 |
| 52255 | | | |
| Predicted folded 24-nts-long seq | 645 | 24 | 64 |
| 52285 | | | |
| Predicted folded 24-nts-long seq | 646 | 24 | 64 |
| 52953 | | | |
| Predicted folded 24-nts-long seq | 647 | 24 | 62 |
| 53693 | | | |
| Predicted siRNA 55507 | 648 | 22 | 59 |
| Predicted siRNA 55629 | 649 | 22 | 62 |
| Predicted siRNA 55775 | 650 | 21 | 61 |
| Predicted siRNA 55869 | 651 | 23 | 61 |
| Predicted siRNA 55937 | 652 | 23 | 60 |
| Predicted siRNA 55979 | 653 | 24 | 60 |
| Predicted siRNA 56066 | 654 | 25 | 59 |
| Predicted siRNA 56759 | 655 | 23 | 59 |
| Predicted siRNA 57049 | 656 | 23 | 61 |
| Predicted siRNA 57283 | 657 | 21 | 66 |
| Predicted siRNA 58170 | 658 | 22 | 60 |
| Predicted siRNA 58574 | 659 | 23 | 59 |
| Predicted siRNA 60433 | 660 | 24 | 66 |
| Predicted siRNA 60529 | 661 | 22 | 61 |
| Predicted zma mir 47934 | 662 | 25 | 60 |
| Predicted zma mir 48043 | 663 | 21 | 61 |
| Predicted zma mir 48120 | 664 | 22 | 61 |
| Predicted zma mir 48193 | 665 | 26 | 60 |
| Predicted zma mir 48408 | 666 | 21 | 58 |
| Predicted zma mir 48451 | 667 | 26 | 60 |
| Predicted zma mir 48462 | 668 | 26 | 59 |
| Predicted zma mir 48514 | 669 | 22 | 61 |
| Predicted zma mir 48520 | 670 | 29 | 59 |
| Predicted zma mir 48653 | 671 | 23 | 58 |
| Predicted zma mir 48669 | 672 | 24 | 60 |
| Predicted zma mir 48682 | 673 | 23 | 59 |
| Predicted zma mir 48841 | 674 | 24 | 59 |
| Predicted zma mir 48966 | 675 | 25 | 61 |
| Predicted zma mir 49156 | 676 | 26 | 60 |
| Predicted zma mir 49199 | 677 | 22 | 60 |
| Predicted zma mir 50109 | 678 | 25 | 59 |
| Predicted zma mir 50425 | 679 | 22 | 60 |
| sbi-miR164c | 680 | 21 | 58 |
| tae-miR1125 | 682 | 24 | 66 |

TABLE 10
Primers for qRT-PCR Analysis of small RNAs
Differentially Expressed in Salt Stress

| | SEQ ID | Primer | |
|---|---|---|---|
| miR Name | NO: | length | Tm |
| Predicted folded 24-nts-long seq | 683 | 24 | 65 |
| 54187 | | | |
| Predicted siRNA 54673 | 684 | 24 | 63 |
| Predicted siRNA 54895 | 685 | 23 | 59 |
| Predicted siRNA 55242 | 686 | 21 | 63 |
| Predicted siRNA 55246 | 687 | 27 | 60 |
| Predicted siRNA 55344 | 688 | 22 | 60 |
| Predicted siRNA 55402 | 689 | 23 | 60 |
| Predicted siRNA 55909 | 690 | 24 | 63 |
| Predicted siRNA 56060 | 691 | 22 | 59 |
| Predicted siRNA 56305 | 692 | 21 | 62 |
| Predicted siRNA 56314 | 693 | 23 | 59 |
| Predicted siRNA 56506 | 694 | 26 | 59 |
| Predicted siRNA 56651 | 695 | 22 | 64 |
| Predicted siRNA 57169 | 696 | 24 | 64 |
| Predicted siRNA 57197 | 697 | 25 | 59 |
| Predicted siRNA 58212 | 698 | 18 | 60 |
| Predicted siRNA 59035 | 699 | 22 | 61 |
| Predicted siRNA 59453 | 700 | 18 | 60 |
| Predicted zma mir 47990 | 701 | 26 | 59 |
| Predicted zma mir 48459 | 702 | 23 | 61 |
| Predicted zma mir 48490 | 703 | 25 | 59 |
| Predicted zma mir 48753 | 704 | 24 | 59 |
| Predicted zma mir 48783 | 705 | 24 | 60 |
| Predicted zma mir 48824 | 706 | 22 | 60 |
| Predicted zma mir 48848 | 707 | 23 | 60 |
| Predicted zma mir 49575 | 708 | 21 | 59 |
| Predicted zma mir 49817 | 709 | 22 | 59 |
| Predicted zma mir 49855 | 710 | 27 | 59 |
| Predicted zma mir 49862 | 711 | 21 | 60 |
| Predicted zma mir 50145 | 712 | 24 | 60 |

TABLE 11
Primers for qRT-PCR Analysis of small RNAs
Differentially Expressed in Heat Stress

| | SEQ ID | Primer | |
|---|---|---|---|
| miR Name | NO: | length | Tm |
| Predicted folded 24-nts-long seq | 713 | 24 | 59 |
| 50957 | | | |
| Predicted folded 24-nts-long seq | 714 | 24 | 64 |
| 51391 | | | |
| Predicted folded 24-nts-long seq | 715 | 24 | 64 |
| 51709 | | | |
| Predicted folded 24-nts-long seq | 716 | 24 | 65 |
| 52606 | | | |
| Predicted folded 24-nts-long seq | 717 | 24 | 65 |
| 52682 | | | |
| Predicted folded 24-nts-long seq | 718 | 24 | 66 |
| 52724 | | | |
| Predicted folded 24-nts-long seq | 719 | 26 | 59 |
| 53851 | | | |
| Predicted folded 24-nts-long seq | 720 | 24 | 64 |
| 53866 | | | |
| Predicted siRNA 54548 | 721 | 24 | 60 |
| Predicted siRNA 54566 | 722 | 22 | 66 |
| Predicted siRNA 54666 | 723 | 22 | 62 |
| Predicted siRNA 54735 | 724 | 24 | 63 |
| Predicted siRNA 55208 | 725 | 26 | 59 |
| Predicted siRNA 55684 | 726 | 22 | 60 |
| Predicted siRNA 55793 | 727 | 25 | 59 |
| Predicted siRNA 55824 | 728 | 23 | 59 |
| Predicted siRNA 55968 | 729 | 24 | 61 |
| Predicted siRNA 56154 | 730 | 24 | 61 |
| Predicted siRNA 56225 | 731 | 24 | 61 |
| Predicted siRNA 56396 | 732 | 24 | 60 |
| Predicted siRNA 56582 | 733 | 21 | 61 |
| Predicted siRNA 56658 | 734 | 24 | 67 |
| Predicted siRNA 56664 | 735 | 21 | 62 |
| Predicted siRNA 56791 | 736 | 26 | 59 |
| Predicted siRNA 56885 | 737 | 24 | 59 |
| Predicted siRNA 57061 | 738 | 22 | 62 |
| Predicted siRNA 57689 | 739 | 20 | 61 |
| Predicted siRNA 58105 | 740 | 24 | 64 |
| Predicted siRNA 58108 | 741 | 20 | 61 |
| Predicted siRNA 58158 | 742 | 21 | 64 |
| Predicted siRNA 58387 | 743 | 27 | 60 |
| Predicted siRNA 58717 | 744 | 24 | 61 |
| Predicted siRNA 58720 | 745 | 20 | 60 |
| Predicted siRNA 58740 | 746 | 24 | 59 |
| Predicted siRNA 59056 | 747 | 23 | 59 |
| Predicted siRNA 59211 | 748 | 21 | 60 |
| Predicted siRNA 59300 | 749 | 20 | 64 |
| Predicted siRNA 59379 | 750 | 20 | 59 |
| Predicted siRNA 59410 | 751 | 22 | 60 |
| Predicted siRNA 59474 | 752 | 19 | 61 |
| Predicted siRNA 59580 | 753 | 24 | 64 |
| Predicted siRNA 59736 | 754 | 19 | 61 |
| Predicted siRNA 59799 | 755 | 20 | 64 |
| Predicted siRNA 59800 | 756 | 20 | 63 |
| Predicted siRNA 59817 | 757 | 21 | 60 |
| Predicted siRNA 59820 | 758 | 20 | 59 |
| Predicted siRNA 59851 | 759 | 22 | 60 |
| Predicted siRNA 59918 | 760 | 21 | 60 |
| Predicted siRNA 59935 | 761 | 25 | 66 |
| Predicted siRNA 59937 | 762 | 25 | 64 |
| Predicted siRNA 59987 | 763 | 22 | 60 |
| Predicted siRNA 60036 | 764 | 24 | 61 |
| Predicted siRNA 60421 | 765 | 27 | 59 |
| Predicted siRNA 60533 | 766 | 20 | 63 |
| Predicted siRNA 60635 | 767 | 29 | 59 |
| Predicted siRNA 60718 | 768 | 25 | 59 |
| Predicted siRNA 60742 | 769 | 24 | 59 |
| Predicted siRNA 60833 | 770 | 21 | 60 |
| Predicted siRNA 60993 | 771 | 25 | 60 |
| Predicted siRNA 61212 | 772 | 21 | 59 |
| Predicted siRNA 61236 | 773 | 25 | 60 |
| Predicted zma mir 47966 | 774 | 26 | 59 |
| Predicted zma mir 48327 | 775 | 22 | 59 |
| Predicted zma mir 48479 | 776 | 24 | 60 |
| Predicted zma mir 48482 | 777 | 24 | 59 |
| Predicted zma mir 48489 | 778 | 22 | 60 |
| Predicted zma mir 48790 | 779 | 22 | 59 |
| Predicted zma mir 48905 | 780 | 25 | 59 |
| Predicted zma mir 49248 | 781 | 21 | 62 |
| Predicted zma mir 49259 | 782 | 22 | 60 |
| Predicted zma mir 49310 | 783 | 21 | 63 |
| Predicted zma mir 49642 | 784 | 22 | 66 |
| Predicted zma mir 49718 | 785 | 22 | 59 |
| Predicted zma mir 49952 | 786 | 23 | 67 |
| Predicted zma mir 50085 | 787 | 25 | 59 |
| Predicted zma mir 50120 | 788 | 23 | 61 |
| Predicted zma mir 50166 | 789 | 22 | 60 |
| Predicted zma mir 50256 | 790 | 22 | 61 |
| Predicted zma mir 50289 | 791 | 19 | 58 |
| Predicted zma mir 50388 | 792 | 24 | 60 |
| Predicted zma mir 50449 | 793 | 23 | 59 |
| Predicted zma mir 50453 | 794 | 22 | 61 |
| Predicted zma mir 50480 | 795 | 20 | 64 |
| Predicted zma mir 50481 | 796 | 20 | 64 |
| Predicted zma mir 50483 | 797 | 20 | 63 |
| Predicted zma mir 50486 | 798 | 22 | 61 |
| Predicted zma mir 50522 | 799 | 22 | 61 |
| Predicted zma mir 50570 | 800 | 20 | 60 |
| Predicted zma mir 50682 | 801 | 23 | 59 |
| Predicted zma mir 50695 | 802 | 22 | 58 |
| Predicted zma mir 50701 | 803 | 19 | 60 |

Alternative RT-PCR Validation Method of Selected microRNAs of the Invention

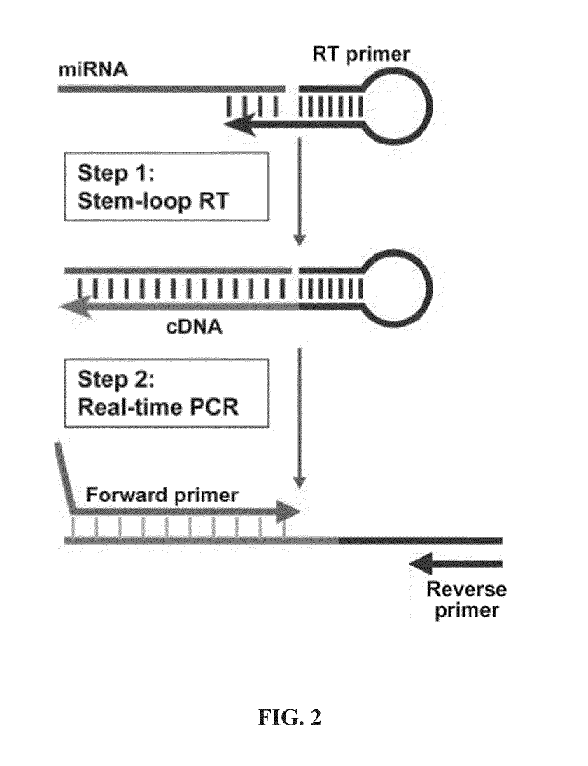

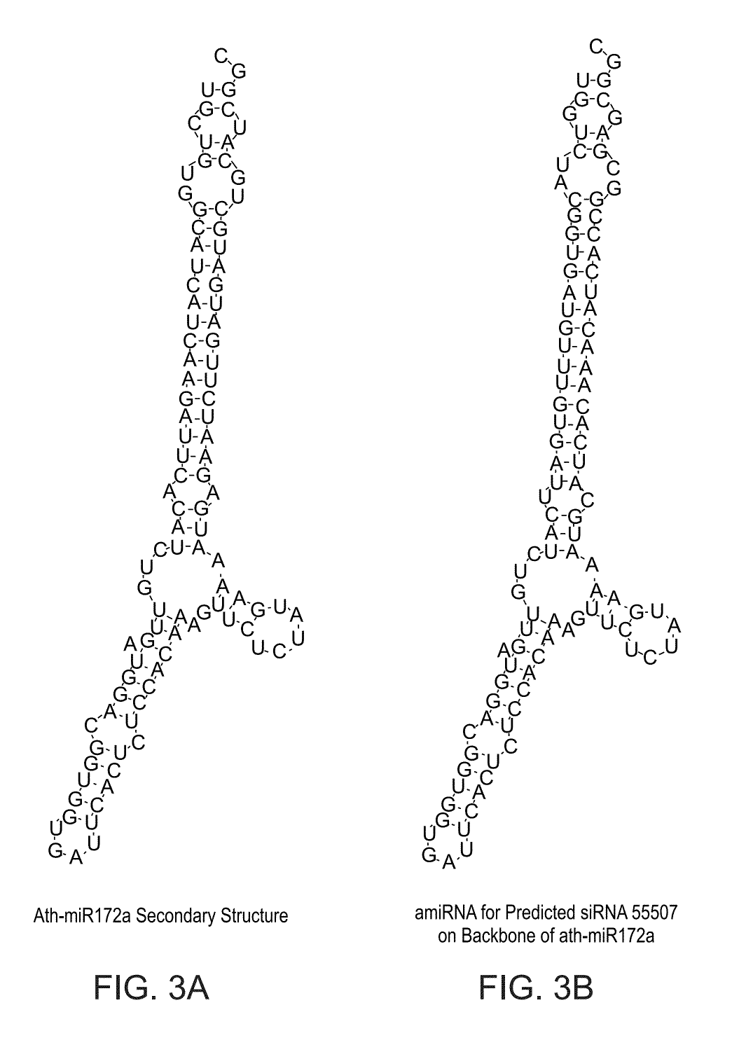

A novel microRNA quantification method has been applied using stem-loop RT followed by PCR analysis (Chen C, Ridzon D A, Broomer A J, Zhou Z, Lee D H, Nguyen J T, Barbisin M, Xu N L, Mahuvakar V R, Andersen M R, Lao K Q, Livak K J, Guegler K J. 2005, Nucleic Acids Res 33(20):e179; Varkonyi-Gasic E, Wu R, Wood M, Walton E F, Hellens R P. 2007, Plant Methods 3:12) (see FIG. 2). This highly accurate method allows the detection of less abundant miRNAs. In this method, stem-loop RT primers are used, which provide higher specificity and efficiency to the reverse transcription process. While the conventional method relies on polyadenylated (poly (A)) tail and thus becomes sensitive to methylation because of the susceptibility of the enzymes involved, in this novel method the reverse transcription step is transcriptspecific and insensitive to methylation. Reverse transcriptase reactions contained RNA samples including purified total RNA, 50 nM stem-loop RT primer (see Tables 12a-c, synthesized by Sigma), and using the SuperScript II reverse transcriptase (Invitrogen). A mix of up to 12 stem-loop RT primers may be used in each reaction, and the forward primers are such that the last 6 nucleotides are replaced with a GC rich sequence. For the PCR step, each miRNA has a custom forward primer, while only miRNAs exhibiting technical difficulties using the stem loop universal reverse primer (5′-GTGCAGGGTCCGAGGT-3′-SEQ ID NO: 228) get custom reverse primer as well. Note, SL-RT stands for stem loop reverse transcription, SL-F are the forward primers, SL-R are the reverse primers.

TABLE 12a
Stem Loop Reverse Transcriptase Primers for RT-PCR Validation
of Differential Mirs under Drought Stress

| | | SEQ ID | Primer |
|---|---|---|---|
| Mir Name | Primer Name | NO: | Length |
| Predicted folded 24- | Pred zma 52255-SL- | 804 | 51 |
| nts-long seq 52255 | RT | | |
| | Pred zma 52255-SL-F | 805 | 21 |
| Predicted siRNA | Pred siRNA 55629-SL- | 806 | 50 |
| 55629 | RT | | |
| | Pred siRNA 55629-SL- | 807 | 20 |
| | F | | |
| Predicted siRNA | Pred zma 55775-SL- | 808 | 50 |
| 55775 | RT | | |
| | Pred zma 55775-SL-F | 809 | 21 |
| Predicted siRNA | Pred zma 55869-SL- | 810 | 50 |
| 55869 | RT | | |
| | Pred zma 55869-SL-F | 811 | 21 |
| Predicted siRNA | Pred zma 55937-SL- | 812 | 50 |
| 55937 | RT | | |
| | Pred zma 55937-SL-F | 813 | 20 |
| Predicted siRNA | Pred zma 58170-SL- | 814 | 50 |
| 58170 | RT | | |
| | Pred zma 58170-SL-F | 815 | 19 |
| Predicted zma mir | Pred zma 47934-SL- | 816 | 50 |
| 47934 | RT | | |
| | Pred zma 47934-SL-F | 817 | 22 |
| Predicted zma mir | Pred zma 48043-SL- | 818 | 50 |
| 48043 | RT | | |
| | Pred zma 48043-SL-F | 819 | 21 |
| Predicted zma mir | Pred zma mir 48193- | 820 | 50 |
| 48193 | SL-RT | | |
| | Pred zma mir 48193- | 821 | 22 |
| | SL-F | | |
| | Pred zma 48193 -SL-R | 822 | 24 |
| Predicted zma mir | Pred zma 48408-SL- | 823 | 50 |
| 48408 | RT | | |
| | Pred zma 48408-SL-F | 824 | 21 |

TABLE 12b
Stem Loop Reverse Transcriptase Primers for RT-PCR Validation
of Differential Mirs under Salinity Stress

| | | Primer | |
|---|---|---|---|
| | | Sequence/ | Primer |
| Mir Name | Primer Name | SEQ ID NO: | Length |
| Predicted siRNA | Pred zma 54895- | 825 | 50 |
| 54895 | SL-RT | | |
| | Pred zma 54895- | 826 | 22 |
| | SL-F | | |
| Predicted siRNA | Pred zma 55344- | 827 | 50 |
| 55344 | SL-RT | | |
| | Pred zma 55344- | 828 | 20 |
| | SL-F | | |
| Predicted siRNA | Pred zma 56506- | 829 | 50 |
| 56506 | SL-RT | | |
| | Pred zma 56506- | 830 | 24 |
| | SL-F | | |
| Predicted siRNA | Pred zma 59035- | 831 | 19 |
| 59035 | SL-F | | |
| | Pred 59035-SL-RT | 832 | 50 |
| Predicted zma mir | Pred zma 47990- | 833 | 50 |
| 47990 | SL-RT | | |
| | Pred zma 47990- | 834 | 22 |
| | SL-F | | |
| Predicted zma mir | Pred zma 48459- | 835 | 51 |
| 48459 | SL-RT | | |
| | Pred zma 48459- | 836 | 18 |
| | SL-F | | |
| Predicted zma mir | Pred zma 49817- | 837 | 51 |
| 49817 | SL-RT | | |
| | Pred zma 49817- | 838 | 18 |
| | SL-F | | |
| Predicted zma mir | Pred zma 49855- | 839 | 50 |
| 49855 | SL-RT | | |
| | Pred zma 49855- | 840 | 21 |
| | SL-F | | |
| Predicted zma mir | Pred zma 49862- | 841 | 18 |
| 49862 | SL-F | | |
| | Pred zma 49862- | 842 | 51 |
| | SL-RT | | |
| Predicted zma mir | Pred zma 50145- | 843 | 50 |
| 50145 | SL-RT | | |
| | Pred zma 50145- | 844 | 20 |
| | SL-F | | |

TABLE 12c
Stem Loop Reverse Transcriptase Primers for RT-PCR Validation
of Differential Mirs under Heat Shock Stress

| | | Primer | |
|---|---|---|---|
| | | Sequence/ | Primer |
| Mir Name | Primer Name | SEQ ID NO: | Length |
| Predicted folded 24-nts-long | Pred zma 53851- | 845 | 50 |
| seq 53851 | SL-RT | | |
| | Pred zma 53851- | 846 | 24 |
| | SL-F | | |
| Predicted folded 24-nts-long | Pred zma 53866- | 847 | 51 |
| seq 53866 | SL-RT | | |
| | Pred zma 53866- | 848 | 23 |
| | SL-F | | |
| Predicted siRNA 54566 | Pred zma 54566- | 849 | 50 |
| | SL-RT | | |
| | Pred zma 54566- | 850 | 20 |
| | SL-F | | |
| Predicted siRNA 55824 | Pred zma 55824- | 851 | 50 |
| | SL-RT | | |
| | Pred zma 55824- | 852 | 20 |
| | SL-F | | |
| Predicted siRNA 58108 | Pred zma 58108- | 853 | 50 |
| | SL-RT | | |
| | Pred zma 58108- | 854 | 17 |
| | SL-F | | |
| Predicted siRNA 59817 | Pred zma 59817- | 855 | 50 |
| | SL-RT | | |
| | Pred zma 59817- | 856 | 19 |
| | SL-F | | |
| Predicted siRNA 59851 | Pred zma 59851- | 857 | 50 |
| | SL-RT | | |
| | Pred zma 59851- | 858 | 18 |
| | SL-F | | |
| Predicted siRNA 59918 | Pred zma 59918- | 859 | 50 |
| | SL-RT | | |
| | Pred zma 59918- | 860 | 20 |
| | SL-F | | |
| Predicted siRNA 59987 | Pred zma 59987- | 861 | 50 |
| | SL-RT | | |
| | Pred zma 59987- | 862 | 20 |
| | SL-F | | |
| | Pred zma 59987- | 863 | 24 |
| | SL-R | | |
| Predicted zma mir 48479 | Pred zma 48479- | 864 | 22 |
| | SL-F | | |
| | Pred zma 48479- | 865 | 50 |
| | SL-RT | | |
| Predicted zma mir 49248 | Pred zma 49248- | 866 | 50 |
| | SL-RT | | |
| | Pred zma 49248- | 867 | 19 |
| | SL-F | | |
| Predicted zma mir 49642 | Pred zma 49642- | 868 | 50 |
| | SL-RT | | |
| | Pred zma 49642- | 869 | 18 |
| | SL-F | | |
| Predicted zma mir 50256 | Pred zma 50256- | 870 | 50 |
| | SL-RT | | |
| | Pred zma 50256- | 871 | 20 |
| | SL-F | | |
| Predicted zma mir 50453 | Pred zma 50453- | 872 | 50 |
| | SL-RT | | |
| | Pred zma 50453- | 873 | 20 |
| | SL-F | | |

### Example 4

### Results of RT-PCR Validation of Selected miRNAs of the Invention

An RT-PCR analysis was run on selected microRNAs of the invention, using the conventional and stem-loop RT primers as described in Tables 9-11 and 12a-c in Example 3 above. Total RNA was extracted from either leaf or root tissues of maize plants grown as described above, and was used as a template for RT-PCR analysis. Expression level and directionality of several up-regulated and down-regulated microRNAs that were found to be differential on the microarray analysis were verified. Results are summarized in Tables 13a-b below.

TABLE 13a
Summary of RT-PCR Verification Results on
Selected miRNAs using Conventional Method

| Trait | miR Name | p-Value | Fold-change |
|---|---|---|---|
| Drought | Predicted folded 24-nts- | 3.80E−03 | 1.82 (−) |
| | long seq 52255 | | |
| | Predicted zma mir | 1.50E−03 | 1.66 (+) |
| | 48043 | | |
| | Predicted siRNA | 5.20E−03 | 1.51 (+) |
| | 55775 | | |

TABLE 13b
Summary of RT-PCR Verification Results on Selected
miRNAs using Stem Loop RT (Alternative) Method

| Trait | miR Name | p-Value | Fold-change |
|---|---|---|---|
| Drought | Predicted folded 24- | 3.30E−02 | 3.12 (−) |
| | nts-long seq 52255 | | |
| | Predicted siRNA 55869 | 5.30E−02 | 2.00 (+) |
| | Predicted siRNA 55629 | 2.70E−02 | 1.43 (+) |
| Salinity | Predicted zma mir 50145 | 4.20E−02 | 1.75 (+) |
| | Predicted zma mir 49817 | 2.20E−03 | 3.28 (−) |
| | Predicted zma mir 47990 | 5.10E−02 | 3.64 (−) |
| | Predicted siRNA 54895 | 1.70E−04 | 3.98 (+) |
| | Predicted siRNA 55344 | 7.40E−06 | 3.31 (+) |
| | Predicted siRNA 56506 | 1.20E−03 | 2.17 (+) |
| | Predicted zma mir 48459 | 1.50E−03 | 4.81 (−) |
| Heat Shock | Predicted siRNA 59987 | 1.20E−04 | 5.40 (+) |
| | Predicted siRNA 54566 | 4.80E−02 | 1.52 (+) |
| | Predicted siRNA 59851 | 7.60E−05 | 10.30 (−) |
| | Predicted zma mir 50453 | 7.40E−05 | 7.24 (−) |
| | Predicted folded 24- | 6.50E−03 | 4.34 (−) |
| | nts-long seq 53866 | | |

### Example 5

### Gene Cloning Strategies for miRNA and siRNA Molecules and Creation of Binary Vectors for Plant Expression

The best validated miRNA sequences are cloned into pORE-E1 binary vectors (FIG. 1) for the generation of transgenic plants. The full-length precursor sequence comprising the hairpin sequence of each selected miRNA, is synthesized by Genscript (USA). The resulting clone is digested with appropriate restriction enzymes and inserted into the Multi Cloning Site (MCS) of a similarly digested binary vector through ligation using T4 DNA ligase enzyme (Promega, Madison, Wis., USA).

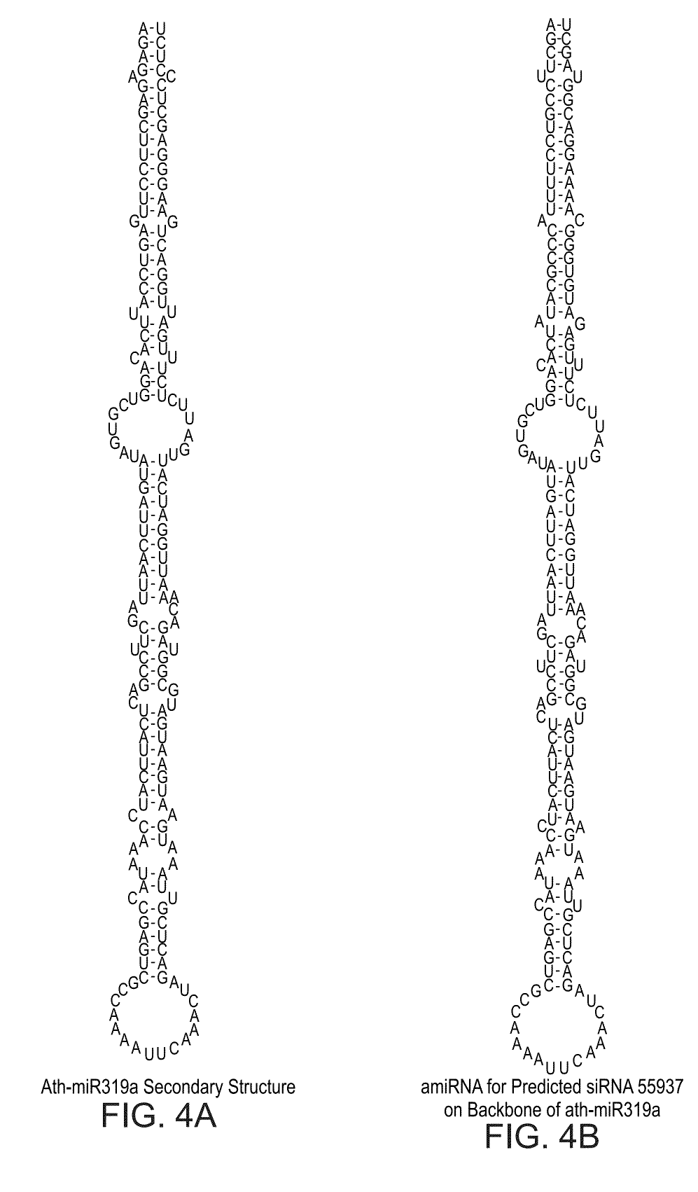

In order to clone siRNA sequences, which have different secondary structures than those of miRNA sequences, a method of artificial microRNA (amiRNA) is implemented, where a plant miRNA precursor is modified to express a small RNA sequence that is not related to the original miRNA produced by the precursor. In this method, the mature siRNA sequence replaces the mature sequence of a specific known miRNA (e.g., miR172a and miR319a) but uses its hairpin backbone for amiRNA expression (Schwab et al., 2006, Plant Cell 18(5): 1121-1133). Moreover, the miRNA* sequences are altered such that both structural and energetic features of the miRNA precursor are retained. Examples for such artificial miRNA constructs using either miR172a (Arabidopsis mature sequence AGAAUCUUGAUGAUGCUGCAU SEQ ID NO: 453, stem loop UGCUGUGGCAUCAUCAAGAUUCACAUCUGUUGAUGGACGGUGGUGAUUC ACUCUCCACAAAGUUCUCUAUGAAAAUGAGAAUCUUGAUGAUGCUGCAU CGGC SEQ ID NO: 454) or miR319a (Arabidopsis mature sequence UUGGACUGAAGGGAGCUCCCU SEQ ID NO: 455, stem loop AGAGAGAGCUUCCUUGAGUCCAUUCACAGGUCGUGAUAUGAUUCAAUUA GCUUCCGACUCAUUCAUCCAAAUACCGAGUCGCCAAAAUUCAAACUAGAC UCGUUAAAUGAAUGAAUGAUGCGGUAGACAAAUUGGAUCAUUGAUUCUC UUUGAUUGGACUGAAGGGAGCUCCCUCU SEQ ID NO: 456), as a backbone are presented in FIGS. 3 and 4, respectively.

### Example 6

### Generation of Transgenic Model Plants Expressing the Abiotic Stress Associated Small RNAs

Arabidoposis thaliana transformation is performed using the floral dip procedure following a slightly modified version of the published protocol (Clough and Bent, 1998, Plant J 16(6): 735-43; and Desfeux et al., 2000, Plant Physiol 123(3): 895-904). Briefly, T0 Plants are planted in small pots filled with soil. The pots are covered with aluminum foil and a plastic dome, kept at 4° C. for 3-4 days, then uncovered and incubated in a growth chamber at 24° C. under 16 hr light: 8 hr dark cycles. A week prior to transformation all individual flowering stems are removed to allow for growth of multiple flowering stems instead. A single colony of Agrobacterium (GV3101) carrying the binary vectors (pORE-E1), harboring the miRNA hairpin sequences with additional flanking sequences both upstream and downstream of it, is cultured in LB medium supplemented with kanamycin (50 mg/L) and gentamycin (25 mg/L). Three days prior to transformation, each culture is incubated at 28° C. for 48 hrs, shaking at 180 rpm. The starter culture is split the day before transformation into two cultures, which are allowed to grow further at 28° C. for 24 hours at 180 rpm. Pellets containing the agrobacterium cells are obtained by centrifugation of the cultures at 5000 rpm for 15 minutes. The pellets are re-suspended in an infiltration medium (10 mM MgCl₂, 5% sucrose, 0.044 μM BAP (Sigma) and 0.03% Tween 20) prepared with double-distilled water.

Transformation of T0 plants is performed by inverting each plant into the agrobacterium suspension, keeping the flowering stem submerged for 5 minutes. Following inoculation, each plant is blotted dry for 5 minutes on both sides, and placed sideways on a fresh covered tray for 24 hours at 22° C. Transformed (transgenic) plants are then uncovered and transferred to a greenhouse for recovery and maturation. The transgenic T0 plants are grown in the greenhouse for 3-5 weeks until the seeds are ready, which are then harvested from plants and kept at room temperature until sowing.

### Example 7

### Selection of Transgenic Arabidopsis Plants Expressing the Abiotic Stress Genes According to Expression Level

Arabidopsis seeds are sown and Basta (Bayer) is sprayed for the first time on 1-2 weeks old seedlings, at least twice every few days. Only resistant plants, which are heterozygous for the transgene, survive. PCR on the genomic gene sequence is performed on the surviving seedlings using primers pORE-F2 (fwd, 5′-TTTAGCGATGAACTTCACTC-3′, SEQ ID NO: 457) and a custom designed reverse primer based on each small RNA sequence.

### Example 8

### Abiotic Stress Tolerance Assessments in Control and Transgenic Plants

Transgenic plants with tolerance to abiotic stress in the form of extreme deficiency in water, high salt concentrations, or heat shock are expected to exhibit better overall survival and growth compared to control non-transgenic plants. Since different plants vary considerably in their tolerance to drought, salinity and heat shock stresses, the duration of drought effected, concentration of salt applied and duration of exposure to high temperature, respectively, can be tailored to the specific plant cultivar or variety (for guidelines specifically to appropriate salt concentrations see, Bernstein and Kafkafi, Root Growth Under Salinity Stress In: Plant Roots, The Hidden Half 3rd ed. Waisel Y, Eshel A and Kafkafi U. (editors) Marcel Dekker Inc., New York, 2002).

Transgenic Arabidopsis plants are allowed to grow until seed production followed by an evaluation of their drought tolerance. Quantitative parameters of tolerance measured include, but are not limited to, the overall size and yield, average wet and dry weight, growth rate, leaf size, leaf coverage (overall leaf area), the weight of the seeds yielded, the average seed size and the number of seeds produced per plant. Under normal conditions, transgenic plants exhibit a phenotype equivalent or superior to that of the wild type plants. Following stress induction i.e., growth under stress, transformed plants not exhibiting substantial physiological and/or morphological effects, or exhibiting higher measured parameters levels compared to wild-type plants, are identified as abiotic stress tolerant plants.

Corn seeds were germinated and grown at 22° C. in soil under normal conditions for 3-4 weeks. Seedlings were then used for experimental assays of each of the following abiotic stresses: drought, salinity and heat shock. Generally, each stress assay includes an internal control group of plants that is continuously grown under optimal conditions. For drought induction, irrigation of the stress group was completely stopped for four or six days. For salinity induction, irrigation with regular water is substituted by irrigation with 300 mM NaCl solution in the stress group, for overall 2-3 irrigations in a period of four or six days. For induction of heat shock, the stress group plants are exposed to a high temperature (37° C.) for one hour. For all stress analyses, tissue samples from both experimental groups are then used for RNA analysis, as described below.

Soil-Based Drought Tolerance Assay

Screens are performed with transgenic plants over-expressing the differential small RNAs detailed above. Briefly, seeds from control Arabidopsis plants, or other transgenic plants over-expressing the small RNA molecule of the invention are germinated and transferred to pots. Drought stress is obtained after irrigation is ceased and the two plant types (transgenic and control plants) are compared when most control plants develop severe wilting, concurrently, rehydration of the plants is initiated at this point. Transgenic plants are ranked on two levels compared to controls: (1) tolerance to drought conditions, and (2) recovery (survival) following re-watering.

To illustrate and elaborate on the above drought tolerance assays of any given wild type plant compared to a corresponding transgenic plant (in which a drought-associated miRNA has been over-expressed), two different approaches are taken as follows:

Lethal drought stress—whereby wild type (used as a control) and transgenic plants (1-3 weeks old) are grown under prolonged extreme drought conditions (duration varies in accordance with plant species). Next, a recovery attempt is implemented during which plants are regularly irrigated and survival level is estimated in the two plant groups 1-2 days post irrigation initiation. While the control (wild type) plant is not expected to survive this extreme stress, the transgenic plant is expected to demonstrate some improved drought tolerance, usually within hours of re-hydration.

Non-lethal drought stress—whereby wild type (used as a control) and transgenic plants (1-3 weeks old) are grown under regular short-term cycles of drought and re-hydration steps, such that re-hydration is applied when general visible drought symptoms (e.g., evident decrease in turgor pressure of lower leaves) emerge in the experimental plants. This drought/irrigation alternating treatment continues until the flowering stage of the plants is reached, followed by an evaluation of dry matter weight. Both wild type and transgenic plants are expected to survive this non-lethal stress, however, measurable differences in drought tolerance are demonstrated by increased yield of the transgenic compared with the wild type plants.

Drought Tolerance Assay Using Sorbitol

Another assay designed to assess whether transgenic plants are more tolerant to drought or severe water deprivation, involves induction of an osmotic stress by the non-ionic osmolyte sorbitol (Mazel et al., 2004, Plant Physiol 134: 118-128). Control and transgenic plants are germinated and grown in plant-agar plates for 4 days, after which they are transferred to plates containing 500 mM sorbitol, to cause delayed growth. Following 7 days of stress treatment, control and transgenic plants are compared by measuring plant weight (wet and dry), yield, and by growth rates measured as time to flowering.

Methods for Salinity Tolerance Assessment

Osmotic stress assays, such as chloride and mannitol assays, are aimed to determine whether an osmotic stress phenotype is sodium chloride-specific or a result of a general osmotic stress. Plants which are tolerant to osmotic stress may also exhibit tolerance to drought and/or freezing. For salt and osmotic stress germination experiments, the medium is supplemented with 50, 100, or 200 mM NaCl or 100 mM, 200 mM NaCl, 400 mM mannitol.

Methods for Heat Stress Tolerance Assessment

Heat stress tolerance is achieved by exposing the plants to temperatures above 34° C. for a certain period, dependent on the plant and in accordance with the above-guidelines. Plant tolerance is examined after transferring the plants back to 22° C. for recovery and evaluation after 5 days relative to internal controls (non-transgenic plants) or plants not exposed to neither cold or heat stress.

Methods for Cold Stress Tolerance Assessment

To analyze cold stress, mature (25 day old) plants are transferred to 4° C. chambers for 1 or 2 weeks, with constitutive light. Next, plants are moved back to the greenhouse for 2 weeks to recover. Following the recovery period, chilling damages such as growth retardation are determined based on measurements of plant weight (wet and dry) and growth rates (e.g. time to flowering, plant size, yield, etc) taken on control and transgenic plants.

### Example 9

### Evaluating Changes in Root Architecture in Transgenic Plants

Many key traits in modern agriculture can be explained by changes in the root architecture of the plant. Root size and depth have been shown to logically correlate with drought tolerance and fertilizer use efficiency, since deeper and more branched root systems provide better coverage of the soil and can access water stored in deeper soil layers.

To test whether the transgenic plants produce a modified root structure, plants can be grown in agar plates placed vertically. A digital picture of the plates is taken every few days and the maximal length and total area covered by the plant roots are assessed. From every construct created, several independent transformation events are checked in replicates. To assess significant differences between root features, statistical test, such as a Student's t-test, is employed in order to identify enhanced root features and to provide a statistical value to the findings.

### Example 10

### Method for Generating Transgenic Maize Plants with Enhanced or Reduced Small RNA Regulation of Target Genes

Target prediction enables two contrasting strategies; an enhancement (positive) or a reduction (negative) of small RNA regulation. Both these strategies have been used in plants and have resulted in significant phenotype alterations. For complete in-vivo assessment of the phenotypic effects of the differential small RNAs of this invention, the inventors plan to implement both over-expression and down-regulation methods on the small RNA molecules found to associate with abiotic stress tolerance as listed in Tables 1-5. In the case of small RNAs that were up-regulated under abiotic stress conditions, an enhancement in abiotic stress tolerance can theoretically be achieved by maintaining their directionality, i.e. over-expressing them. Conversely, in the case of small RNAs that were down-regulated under abiotic stress conditions, enhancement in tolerance can be achieved by reduction of their regulation. Reduction of small RNA regulation of target genes can be accomplished in one of two approaches:

Expressing a miRNA-Resistant Target

In this method, silent mutations are introduced in the miRNA binding site of the target gene so that the DNA and resulting RNA sequences are changed to prevent miRNA binding, but the amino acid sequence of the protein is unchanged.

For design of miRNA-resistant target sequences for the small RNA molecules of the invention, optimization of the nucleic acid sequence in accordance with the preferred codon usage for a particular plant species is required. Tables such as those provided on-line at the Codon Usage Database through the NCBI (National Center for Biotechnology Information) webpage (Hypertext Transfer Protocol://World Wide Web (dot)ncbi(dot)nlm(dot)nih(dot)gov/Taxonomy/Utils/wprintgc(dot)cgi) were used. The Genbank database contains codon usage tables for a number of different species, with its Table 11 (The Bacterial, Archaeal and Plant Plastid Code) being the most relevant for plant species of this invention.

Expressing a Target-Mimic Sequence

Plant miRNAs usually lead to cleavage of their targeted gene, with this cleavage typically occurring between bases 10 and 11 of the miRNA. This position is therefore especially sensitive to mismatches between the miRNA and the target. It was found that expressing a DNA sequence that could potentially be targeted by a miRNA, but contains three extra nucleotides (ATC), and thus creating a bulge in a key position (between the two nucleotides that are predicted to hybridize with bases 10-11 of the miRNA), can inhibit the regulation of that miRNA on its native targets (Franco-Zorilla et al., 2007, Nat Genet. 39(8):1033-1037).

This type of sequence is referred to as a “target-mimic”. Inhibition of the miRNA regulation is presumed to occur through physically capturing the miRNA by the target-mimic sequence and titering-out the miRNA, thereby reducing its abundance. This method was used to reduce the amount and, consequentially, the regulation of miRNA 399 in Arabidopsis.

Tables 14-16 below present miRNA-resistant target examples and Tables 17-19 below present target mimic examples for differential (downregulated) miRNAs under drought, salinity and heat-shock stress, respectively.

TABLE 14
miRNA-Resistant Target Examples for miRNAs which
were Downregulated under Drought Stress.

| | | Original | ORF | Mutated | |
|---|---|---|---|---|---|
| | Protein | Nucleotide | nucleotide | Nucleotide | NCBI Mir |
| Mir | Sequence/ | Sequence/ | seq/ | Sequence/ | Binding |
| name | SEQ ID NO: | SEQ ID NO: | SEQ ID NO: | SEQ ID NO: | Site |
| Predicted | 874 | 875 | 876 | 877 | 1224-1245 |
| siRNA | | | | 878 | 1224-1245 |
| 55937 | | | | 879 | 1224-1245 |
| | | | | 880 | 1224-1245 |
| | | | | 881 | 1224-1245 |
| | | | | 882 | 1224-1245 |
| | | | | 883 | 1224-1245 |
| | | | | 884 | 1224-1245 |
| | | | | 885 | 1224-1245 |
| | | | | 886 | 1224-1245 |

TABLE 15a
miRNA-Resistant Target Examples for miRNAs which
were Downregulated under Salinity Stress.

| | Original | ORF | Mutated | |
|---|---|---|---|---|
| | Nucleotide | nucleotide | Nucleotide | NCBI Mir |
| Mir | Sequence/ | seq/ | Sequence/ | Binding |
| name | SEQ ID NO: | SEQ ID NO: | SEQ ID NO: | Site |
| Predicted | 887 | 890 | 893 | 100-120 |
| zma mir | | | 894 | 100-120 |
| 48459 | | | 895 | 100-120 |
| | | | 896 | 100-120 |
| | | | 897 | 100-120 |
| | | | 898 | 100-120 |
| | | | 899 | 100-120 |
| | | | 900 | 100-120 |
| | | | 901 | 100-120 |
| | | | 902 | 100-120 |
| Predicted | 888 | 891 | 903 | 414-434 |
| zma mir | | | 904 | 414-434 |
| 48824 | | | 905 | 414-434 |
| | | | 906 | 414-434 |
| | | | 907 | 414-434 |
| | | | 908 | 414-434 |
| | | | 909 | 414-434 |
| Predicted | 889 | 892 | 910 | 515-535 |
| zma mir | | | 911 | 515-535 |
| 49862 | | | 912 | 515-535 |
| | | | 913 | 515-535 |

TABLE 15b
miRNA-Resistant Target Examples for miRNAs which
were Upregulated under Salinity Stress

| | Original | ORF | Mutated | |
|---|---|---|---|---|
| | Nucleotide | nucleotide | Nucleotide | NCBI Mir |
| Mir | Sequence/ | seq/ | Sequence/ | Binding |
| name | SEQ ID NO: | SEQ ID NO: | SEQ ID NO: | Site |
| Predicted | 914 | 923 | 932 | 280-301 |
| siRNA | | | 933 | 280-301 |
| 56314 | | | 934 | 280-301 |
| | | | 935 | 280-301 |
| | | | 936 | 280-301 |
| | | | 937 | 280-301 |
| | | | 938 | 280-301 |
| | | | 939 | 280-301 |
| | | | 940 | 280-301 |
| | | | 941 | 280-301 |
| Predicted | 915 | 924 | 942 | 176-193 |
| siRNA | | | 943 | 176-193 |
| 58212 | | | 944 | 176-193 |
| | | | 945 | 176-193 |
| | 916 | 925 | 946 | 970-987 |
| | | | 947 | 970-987 |
| | | | 948 | 970-987 |
| | | | 949 | 970-987 |
| | | | 950 | 970-987 |
| | | | 951 | 970-987 |
| | | | 952 | 970-987 |
| | 917 | 926 | 953 | 1294-1311 |
| | | | 954 | 1294-1311 |
| | | | 955 | 1294-1311 |
| | | | 956 | 1294-1311 |
| | | | 957 | 1294-1311 |
| | | | 958 | 1294-1311 |
| | | | 959 | 1294-1311 |
| | | | 960 | 1294-1311 |
| | | | 961 | 1294-1311 |
| | | | 962 | 1294-1311 |
| | 918 | 927 | 963 | 183-200 |
| | | | 964 | 183-200 |
| | | | 965 | 183-200 |
| | | | 966 | 183-200 |
| | | | 967 | 183-200 |
| | | | 968 | 183-200 |
| | | | 969 | 183-200 |
| | | | 970 | 183-200 |
| | | | 971 | 183-200 |
| | | | 972 | 183-200 |
| | 919 | 928 | 973 | 130-147 |
| | | | 974 | 130-147 |
| | | | 975 | 130-147 |
| | | | 976 | 130-147 |
| | | | 977 | 130-147 |
| | | | 978 | 130-147 |
| | | | 979 | 130-147 |
| | | | 980 | 130-147 |
| | | | 981 | 130-147 |
| | | | 982 | 130-147 |
| Predicted | 920 | 929 | 983 | 527-544 |
| siRNA | | | 984 | 527-544 |
| 59453 | | | 985 | 527-544 |
| | | | 986 | 527-544 |
| | | | 987 | 527-544 |
| | | | 988 | 527-544 |
| | | | 989 | 527-544 |
| | | | 990 | 527-544 |
| | | | 991 | 527-544 |
| | | | 992 | 527-544 |
| | 921 | 930 | 993 | 1165-1182 |
| | | | 994 | 1165-1182 |
| | | | 995 | 1165-1182 |
| | | | 996 | 1165-1182 |
| | | | 997 | 1165-1182 |
| | | | 998 | 1165-1182 |
| | | | 999 | 1165-1182 |
| | | | 1000 | 1165-1182 |
| | | | 1001 | 1165-1182 |
| | | | 1002 | 1165-1182 |
| | 922 | 931 | 1003 | 1135-1152 |
| | | | 1004 | 1135-1152 |
| | | | 1005 | 1135-1152 |
| | | | 1006 | 1135-1152 |
| | | | 1007 | 1135-1152 |
| | | | 1008 | 1135-1152 |
| | | | 1009 | 1135-1152 |
| | | | 1010 | 1135-1152 |
| | | | 1011 | 1135-1152 |
| | | | 1012 | 1135-1152 |

TABLE 16a
miRNA-Resistant Target Examples for miRNAs which were Downregulated during Heat Shock.

| | | | | | Original | ORF | Mutated | NCBI |
|---|---|---|---|---|---|---|---|---|
| | Mir | Homolog | | Protein | Nucleotide | nucleotide | Nucleotide | Mir |
| Mir | sequence/ | NCBI | | Sequence/ | Sequence/ | seq/ | Sequence/ | Binding |
| name | SEQ ID NO: | Accession | Organism | SEQ ID NO: | SEQ ID NO: | SEQ ID NO: | SEQ ID NO: | Site |
| Predicted | 59 | ACR33787 | Zea mays | 1013 | 1084 | 1155 | | |
| siRNA 57689 | | | | | | | 1226 | 822-839 |
| | | | | | | | 1227 | 822-839 |
| | | | | | | | 1228 | 822-839 |
| | | | | | | | 1229 | 822-839 |
| | | | | | | | 1230 | 822-839 |
| | | ACF83391 | Zea mays | 1014 | 1085 | 1156 | | |
| | | | | | | | 1231 | 575-592 |
| | | | | | | | 1232 | 575-592 |
| | | | | | | | 1233 | 575-592 |
| | | | | | | | 1234 | 575-592 |
| | | | | | | | 1235 | 575-592 |
| Predicted | 60 | XP_002455452 | Sorghum bicolor | 1015 | 1086 | 1157 | | |
| siRNA 58108 | | | | | | | 1236 | 617-634 |
| | | | | | | | 1237 | 617-634 |
| | | | | | | | 1238 | 617-634 |
| | | | | | | | 1239 | 617-634 |
| | | | | | | | 1240 | 617-634 |
| | | NP_001132904 | Zea mays | 1016 | 1087 | 1158 | | |
| | | | | | | | 1241 | 1540-1557 |
| | | | | | | | 1242 | 1540-1557 |
| | | | | | | | 1243 | 1540-1557 |
| | | | | | | | 1244 | 1540-1557 |
| | | | | | | | 1245 | 1540-1557 |
| | | XP_002451348 | Sorghum bicolor | 1017 | 1088 | 1159 | | |
| | | | | | | | 1246 | 1564-1581 |
| | | | | | | | 1247 | 1564-1581 |
| | | | | | | | 1248 | 1564-1581 |
| | | NP_001143089 | Zea mays | 1018 | 1089 | 1160 | | |
| | | | | | | | 1249 | 1528-1545 |
| | | | | | | | 1250 | 1528-1545 |
| | | | | | | | 1251 | 1528-1545 |
| | | | | | | | 1252 | 1528-1545 |
| | | | | | | | 1253 | 1528-1545 |
| Predicted | 61 | ACL53547 | Zea mays | 1019 | 1090 | 1161 | | |
| siRNA 58158 | | | | | | | 1254 | 1179-1199 |
| | | | | | | | 1255 | 1179-1199 |
| | | | | | | | 1256 | 1179-1199 |
| | | | | | | | 1257 | 1179-1199 |
| | | XP_002460562 | Sorghum bicolor | 1020 | 1091 | 1162 | | |
| | | | | | | | 1258 | 1382-1402 |
| | | | | | | | 1259 | 1382-1402 |
| | | | | | | | 1260 | 1382-1402 |
| | | | | | | | 1261 | 1382-1402 |
| | | | | | | | 1262 | 1382-1402 |
| | | ACN31936 | Zea mays | 1021 | 1092 | 1163 | | |
| | | | | | | | 1263 | 1393-1413 |
| | | | | | | | 1264 | 1393-1413 |
| | | | | | | | 1265 | 1393-1413 |
| | | | | | | | 1266 | 1393-1413 |
| | | NP_001183878 | Zea mays | 1022 | 1093 | 1164 | | |
| | | | | | | | 1267 | 1180-1200 |
| | | | | | | | 1268 | 1180-1200 |
| | | | | | | | 1269 | 1180-1200 |
| | | | | | | | 1270 | 1180-1200 |
| Predicted | 64 | NP_001169291 | Zea mays | 1023 | 1094 | 1165 | | |
| siRNA 59056 | | | | | | | 1271 | 353-371 |
| | | | | | | | 1272 | 353-371 |
| | | | | | | | 1273 | 353-371 |
| | | | | | | | 1274 | 353-371 |
| | | XP_002437665 | Sorghum bicolor | 1024 | 1095 | 1166 | | |
| | | | | | | | 1275 | 264-282 |
| | | | | | | | 1276 | 264-282 |
| | | | | | | | 1277 | 264-282 |
| | | | | | | | 1278 | 264-282 |
| | | | | | | | 1279 | 264-282 |
| | | ACL52777 | Zea mays | 1025 | 1096 | 1167 | | |
| | | | | | | | 1280 | 30-Dec |
| | | | | | | | 1281 | 30-Dec |
| | | | | | | | 1282 | 30-Dec |
| | | | | | | | 1283 | 30-Dec |
| | | | | | | | 1284 | 30-Dec |
| | | NP_001140626 | Zea mays | 1026 | 1097 | 1168 | | |
| | | | | | | | 1285 | 328-346 |
| | | | | | | | 1286 | 328-346 |
| | | | | | | | 1287 | 328-346 |
| | | | | | | | 1288 | 328-346 |
| | | | | | | | 1289 | 328-346 |
| | | NP_001143033 | Zea mays | 1027 | 1098 | 1169 | | |
| | | | | | | | 1290 | 60-78 |
| | | | | | | | 1291 | 60-78 |
| | | | | | | | 1292 | 60-78 |
| | | | | | | | 1293 | 60-78 |
| | | | | | | | 1294 | 60-78 |
| | | NP_001146570 | Zea mays | 1028 | 1099 | 1170 | | |
| | | | | | | | 1295 | 1713-1731 |
| | | | | | | | 1296 | 1713-1731 |
| | | | | | | | 1297 | 1713-1731 |
| | | | | | | | 1298 | 1713-1731 |
| | | | | | | | 1299 | 1713-1731 |
| Predicted | 66 | XP_002453411 | Sorghum bicolor | 1029 | 1100 | 1171 | | |
| siRNA 59300 | | | | | | | 1300 | 2064-2083 |
| | | NP_001144625 | Zea mays | 1030 | 1101 | 1172 | | |
| | | | | | | | 1301 | 237-256 |
| | | | | | | | 1302 | 237-256 |
| | | | | | | | 1303 | 237-256 |
| | | | | | | | 1304 | 237-256 |
| | | | | | | | 1305 | 237-256 |
| | | ADX60172 | Zea mays | 1031 | 1102 | 1173 | | |
| | | | | | | | 1306 | 1838-1857 |
| Predicted | 67 | XP_002464695 | Sorghum bicolor | 1032 | 1103 | 1174 | | |
| siRNA 59379 | | | | | | | 1307 | 35-52 |
| | | | | | | | 1308 | 35-52 |
| | | | | | | | 1309 | 35-52 |
| | | | | | | | 1310 | 35-52 |
| Predicted | 69 | XP_002466400 | Sorghum bicolor | 1033 | 1104 | 1175 | | |
| siRNA 59474 | | | | | | | 1311 | 278-296 |
| | | | | | | | 1312 | 278-296 |
| | | | | | | | 1313 | 278-296 |
| | | | | | | | 1314 | 278-296 |
| | | NP_001151137 | Zea mays | 1034 | 1105 | 1176 | | |
| | | | | | | | 1315 | 597-615 |
| | | | | | | | 1316 | 597-615 |
| | | | | | | | 1317 | 597-615 |
| | | | | | | | 1318 | 597-615 |
| | | | | | | | 1319 | 597-615 |
| | | ACG50012 | Zea mays | 1035 | 1106 | 1177 | | |
| | | | | | | | 1320 | 41-59 |
| | | | | | | | 1321 | 41-59 |
| | | | | | | | 1322 | 41-59 |
| | | | | | | | 1323 | 41-59 |
| | | XP_002465546 | Sorghum bicolor | 1036 | 1107 | 1178 | | |
| | | | | | | | 1324 | 387-405 |
| | | | | | | | 1325 | 387-405 |
| | | | | | | | 1326 | 387-405 |
| | | | | | | | 1327 | 387-405 |
| | | | | | | | 1328 | 387-405 |
| | | NP_001149657 | Zea mays | 1037 | 1108 | 1179 | | |
| | | | | | | | 1329 | 279-297 |
| | | | | | | | 1330 | 279-297 |
| | | | | | | | 1331 | 279-297 |
| | | | | | | | 1332 | 279-297 |
| Predicted | 70 | ACF84208 | Zea mays | 1038 | 1109 | 1180 | | |
| siRNA 59736 | | | | | | | 1333 | 491-509 |
| | | | | | | | 1334 | 491-509 |
| | | | | | | | 1335 | 491-509 |
| | | | | | | | 1336 | 491-509 |
| | | | | | | | 1337 | 491-509 |
| | | NP_001183626 | Zea mays | 1039 | 1110 | 1181 | | |
| | | | | | | | 1338 | 225-243 |
| | | | | | | | 1339 | 225-243 |
| | | | | | | | 1340 | 225-243 |
| | | | | | | | 1341 | 225-243 |
| | | | | | | | 1342 | 225-243 |
| Predicted | 71 | ACR34837 | Zea mays | 1040 | 1111 | 1182 | | |
| siRNA 59799 | | | | | | | 1343 | 154-173 |
| Predicted | 72 | NP_001132616 | Zea mays | 1041 | 1112 | 1183 | | |
| siRNA 59800 | | | | | | | 1344 | 343-362 |
| | | | | | | | 1345 | 343-362 |
| | | | | | | | 1346 | 343-362 |
| | | | | | | | 1347 | 343-362 |
| | | | | | | | 1348 | 343-362 |
| Predicted | 75 | XP_002458387 | Sorghum bicolor | 1042 | 1113 | 1184 | | |
| siRNA 59918 | | | | | | | 1349 | 801-821 |
| | | | | | | | 1350 | 801-821 |
| | | | | | | | 1351 | 801-821 |
| | | | | | | | 1352 | 801-821 |
| | | | | | | | 1353 | 801 -821 |
| Predicted zma | 91 | NP_001183850 | Zea mays | 1043 | 1114 | 1185 | | |
| mir 50289 | | | | | | | 1354 | 1414-1432 |
| | | | | | | | 1355 | 1414-1432 |
| | | | | | | | 1356 | 1414-1432 |
| | | | | | | | 1357 | 1414-1432 |
| | | | | | | | 1358 | 1414-1432 |
| | | NP_001168893 | Zea mays | 1044 | 1115 | 1186 | | |
| | | | | | | | 1359 | 1395-1413 |
| Predicted zma | 94 | NP_001147862 | Zea mays | 1045 | 1116 | 1187 | | |
| mir 50480 | | | | | | | 1360 | 626-645 |
| | | | | | | | 1361 | 626-645 |
| | | | | | | | 1362 | 626-645 |
| | | | | | | | 1363 | 626-645 |
| | | | | | | | 1364 | 626-645 |
| | | ACN27570 | Zea mays | 1046 | 1117 | 1188 | | |
| | | | | | | | 1365 | 1304-1323 |
| | | | | | | | 1366 | 1304-1323 |
| | | | | | | | 1367 | 1304-1323 |
| | | | | | | | 1368 | 1304-1323 |
| | | NP_001151285 | Zea mays | 1047 | 1118 | 1189 | | |
| | | | | | | | 1369 | 670-689 |
| | | | | | | | 1370 | 670-689 |
| | | | | | | | 1371 | 670-689 |
| | | | | | | | 1372 | 670-689 |
| | | | | | | | 1373 | 670-689 |
| | | NP_001168251 | Zea mays | 1048 | 1119 | 1190 | | |
| | | | | | | | 1374 | 458-477 |
| | | | | | | | 1375 | 458-477 |
| | | | | | | | 1376 | 458-477 |
| | | | | | | | 1377 | 458-477 |
| | | | | | | | 1378 | 458-477 |
| | | ACN27595 | Zea mays | 1049 | 1120 | 1191 | | |
| | | | | | | | 1379 | 858-877 |
| | | | | | | | 1380 | 858-877 |
| | | | | | | | 1381 | 858-877 |
| | | | | | | | 1382 | 858-877 |
| | | | | | | | 1383 | 858-877 |
| | | NP_001159284 | Zea mays | 1050 | 1121 | 1192 | | |
| | | | | | | | 1384 | 1743-1762 |
| | | | | | | | 1385 | 1743-1762 |
| | | | | | | | 1386 | 1743-1762 |
| | | | | | | | 1387 | 1743-1762 |
| | | | | | | | 1388 | 1743-1762 |
| | | NP_001159342 | Zea mays | 1051 | 1122 | 1193 | | |
| | | | | | | | 1389 | 1751-1770 |
| | | | | | | | 1390 | 1751-1770 |
| | | | | | | | 1391 | 1751-1770 |
| | | | | | | | 1392 | 1751-1770 |
| | | | | | | | 1393 | 1751-1770 |
| | | NP_001146934 | Zea mays | 1052 | 1123 | 1194 | | |
| | | | | | | | 1394 | 151-170 |
| | | | | | | | 1395 | 151-170 |
| | | | | | | | 1396 | 151-170 |
| | | | | | | | 1397 | 151-170 |
| | | XP_002449308 | Sorghum bicolor | 1053 | 1124 | 1195 | | |
| | | | | | | | 1398 | 409-428 |
| | | | | | | | 1399 | 409-428 |
| | | | | | | | 1400 | 409-428 |
| | | | | | | | 1401 | 409-428 |
| | | | | | | | 1402 | 409-428 |
| | | NP_001144625 | Zea mays | 1054 | 1125 | 1196 | | |
| | | | | | | | 1403 | 237-256 |
| | | | | | | | 1404 | 237-256 |
| | | | | | | | 1405 | 237-256 |
| | | | | | | | 1406 | 237-256 |
| | | | | | | | 1407 | 237-256 |
| | | NP_001151654 | Zea mays | 1055 | 1126 | 1197 | | |
| | | | | | | | 1408 | 118-137 |
| | | | | | | | 1409 | 118-137 |
| | | | | | | | 1410 | 118-137 |
| | | | | | | | 1411 | 118-137 |
| | | | | | | | 1412 | 118-137 |
| Predicted zma | 95 | NP_001140853 | Zea mays | 1056 | 1127 | 1198 | | |
| mir 50481 | | | | | | | 1413 | 506-525 |
| | | | | | | | 1414 | 506-525 |
| | | | | | | | 1415 | 506-525 |
| | | | | | | | 1416 | 506-525 |
| | | | | | | | 1417 | 506-525 |
| | | ACN35719 | Zea mays | 1057 | 1128 | 1199 | | |
| | | | | | | | 1418 | 669-688 |
| | | | | | | | 1419 | 669-688 |
| | | | | | | | 1420 | 669-688 |
| | | | | | | | 1421 | 669-688 |
| | | | | | | | 1422 | 669-688 |
| | | NP_001130351 | Zea mays | 1058 | 1129 | 1200 | | |
| | | | | | | | 1423 | 1174-1193 |
| | | | | | | | 1424 | 1174-1193 |
| | | | | | | | 1425 | 1174-1193 |
| | | | | | | | 1426 | 1174-1193 |
| | | | | | | | 1427 | 1174-1193 |
| | | ACF84329 | Zea mays | 1059 | 1130 | 1201 | | |
| | | | | | | | 1428 | 223-242 |
| | | | | | | | 1429 | 223-242 |
| | | | | | | | 1430 | 223-242 |
| | | | | | | | 1431 | 223-242 |
| | | | | | | | 1432 | 223-242 |
| | | XP_002463817 | Sorghum bicolor | 1060 | 1131 | 1202 | | |
| | | | | | | | 1433 | 1298-1317 |
| | | | | | | | 1434 | 1298-1317 |
| | | | | | | | 1435 | 1298-1317 |
| | | | | | | | 1436 | 1298-1317 |
| | | XP_002465627 | Sorghum bicolor | 1061 | 1132 | 1203 | | |
| | | | | | | | 1437 | 1990-2009 |
| | | | | | | | 1438 | 1990-2009 |
| | | | | | | | 1439 | 1990-2009 |
| | | | | | | | 1440 | 1990-2009 |
| | | | | | | | 1441 | 1990-2009 |
| | | NP_001105211 | Zea mays | 1062 | 1133 | 1204 | | |
| | | | | | | | 1442 | 911-930 |
| | | | | | | | 1443 | 911-930 |
| | | | | | | | 1444 | 911-930 |
| | | | | | | | 1445 | 911-930 |
| | | | | | | | 1446 | 911-930 |
| | | NP_001158910 | Zea mays | 1063 | 1134 | 1205 | | |
| | | | | | | | 1447 | 491-510 |
| | | | | | | | 1448 | 491-510 |
| | | | | | | | 1449 | 491-510 |
| | | | | | | | 1450 | 491-510 |
| | | ACF84241 | Zea mays | 1064 | 1135 | 1206 | | |
| | | | | | | | 1451 | 1477-1496 |
| | | | | | | | 1452 | 1477-1496 |
| | | | | | | | 1453 | 1477-1496 |
| | | | | | | | 1454 | 1477-1496 |
| | | NP_001132755 | Zea mays | 1065 | 1136 | 1207 | | |
| | | | | | | | 1455 | 591-610 |
| | | | | | | | 1456 | 591-610 |
| | | | | | | | 1457 | 591-610 |
| | | | | | | | 1458 | 591-610 |
| | | | | | | | 1459 | 591-610 |
| | | NP_001144625 | Zea mays | 1066 | 1137 | 1208 | | |
| | | | | | | | 1460 | 237-256 |
| | | | | | | | 1461 | 237-256 |
| | | | | | | | 1462 | 237-256 |
| | | | | | | | 1463 | 237-256 |
| | | | | | | | 1464 | 237-256 |
| | | XP_002453372 | Sorghum bicolor | 1067 | 1138 | 1209 | | |
| | | | | | | | 1465 | 26-45 |
| | | | | | | | 1466 | 26-45 |
| | | | | | | | 1467 | 26-45 |
| | | | | | | | 1468 | 26-45 |
| | | | | | | | 1469 | 26-45 |
| Predicted zma | 96 | XP_002439337 | Sorghum bicolor | 1068 | 1139 | 1210 | | |
| mir 50483 | | | | | | | 1470 | 592-611 |
| | | ACF80701 | Zea mays | 1069 | 1140 | 1211 | | |
| | | | | | | | 1471 | 897-916 |
| | | | | | | | 1472 | 897-916 |
| | | | | | | | 1473 | 897-916 |
| | | | | | | | 1474 | 897-916 |
| | | | | | | | 1475 | 897-916 |
| | | XP_002455492 | Sorghum bicolor | 1070 | 1141 | 1212 | | |
| | | | | | | | 1476 | 342-361 |
| | | | | | | | 1477 | 342-361 |
| | | | | | | | 1478 | 342-361 |
| | | | | | | | 1479 | 342-361 |
| | | | | | | | 1480 | 342-361 |
| | | NP_001142046 | Zea mays | 1071 | 1142 | 1213 | | |
| | | | | | | | 1481 | 360-379 |
| | | | | | | | 1482 | 360-379 |
| | | | | | | | 1483 | 360-379 |
| | | | | | | | 1484 | 360-379 |
| | | XP_002451328 | Sorghum bicolor | 1072 | 1143 | 1214 | | |
| | | | | | | | 1485 | 1185-1204 |
| | | | | | | | 1486 | 1185-1204 |
| | | | | | | | 1487 | 1185-1204 |
| | | | | | | | 1488 | 1185-1204 |
| | | | | | | | 1489 | 1185-1204 |
| | | NP_001142230 | Zea mays | 1073 | 1144 | 1215 | | |
| | | | | | | | 1490 | 199-218 |
| | | ACN26514 | Zea mays | 1074 | 1145 | 1216 | | |
| | | | | | | | 1491 | 429-448 |
| | | | | | | | 1492 | 429-448 |
| | | | | | | | 1493 | 429-448 |
| | | | | | | | 1494 | 429-448 |
| | | | | | | | 1495 | 429-448 |
| | | NP_001152292 | Zea mays | 1075 | 1146 | 1217 | | |
| | | | | | | | 1496 | 230-249 |
| | | | | | | | 1497 | 230-249 |
| | | | | | | | 1498 | 230-249 |
| | | | | | | | 1499 | 230-249 |
| | | | | | | | 1500 | 230-249 |
| | | ACF81426 | Zea mays | 1076 | 1147 | 1218 | | |
| | | | | | | | 1501 | 690-709 |
| | | | | | | | 1502 | 690-709 |
| | | | | | | | 1503 | 690-709 |
| | | | | | | | 1504 | 690-709 |
| | | | | | | | 1505 | 690-709 |
| | | NP_001141352 | Zea mays | 1077 | 1148 | 1219 | | |
| | | | | | | | 1506 | 240-259 |
| | | | | | | | 1507 | 240-259 |
| | | | | | | | 1508 | 240-259 |
| | | | | | | | 1509 | 240-259 |
| | | | | | | | 1510 | 240-259 |
| | | XP_002437204 | Sorghum bicolor | 1078 | 1149 | 1220 | | |
| | | | | | | | 1511 | 447-466 |
| | | | | | | | 1512 | 447-466 |
| | | | | | | | 1513 | 447-466 |
| | | | | | | | 1514 | 447-466 |
| | | | | | | | 1515 | 447-466 |
| | | NP_001141965 | Zea mays | 1079 | 1150 | 1221 | | |
| | | | | | | | 1516 | 993-1012 |
| | | | | | | | 1517 | 993-1012 |
| | | | | | | | 1518 | 993-1012 |
| | | | | | | | 1519 | 993-1012 |
| | | | | | | | 1520 | 993-1012 |
| | | NP_001130136 | Zea mays | 1080 | 1151 | 1222 | | |
| | | | | | | | 1521 | 750-769 |
| | | | | | | | 1522 | 750-769 |
| | | | | | | | 1523 | 750-769 |
| | | | | | | | 1524 | 750-769 |
| | | | | | | | 1525 | 750-769 |
| | | XP_002464517 | Sorghum bicolor | 1081 | 1152 | 1223 | | |
| | | | | | | | 1526 | 345-364 |
| | | | | | | | 1527 | 345-364 |
| | | | | | | | 1528 | 345-364 |
| | | | | | | | 1529 | 345-364 |
| | | | | | | | 1530 | 345-364 |
| | | NP_001147443 | Zea mays | 1082 | 1153 | 1224 | | |
| | | | | | | | 1531 | 830-849 |
| Predicted zma | 98 | ACG38830 | Zea mays | 1083 | 1154 | 1225 | | |
| mir 50682 | | | | | | | 1532 | 561-580 |
| | | | | | | | 1533 | 561-580 |
| | | | | | | | 1534 | 561-580 |
| | | | | | | | 1535 | 561-580 |

TABLE 16b
miRNA-Resistant Target Examples for miRNAs which were Upregulated during Heat Shock

| | | | | | Original | ORF | Mutated | |
|---|---|---|---|---|---|---|---|---|
| | | Homolog | | Protein | Nucleotide | nucleotide | Nucleotide | NCBI Mir |
| Mir | | NCBI | | Sequence/ | Sequence/ | seq/ | Sequence/ | Binding |
| name | SEQ ID NO: | Accession | Organism | SEQ ID NO: | SEQ ID NO: | SEQ ID NO: | SEQ ID NO: | Site |
| Predicted folded | 177 | NP_001169556 | Zea mays | 1536 | 1563 | 1590 | | |
| 24-nts-long | | | | | | | 1617 | 1561-1584 |
| seq 52606 | | | | | | | 1618 | 1561-1584 |
| | | | | | | | 1619 | 1561-1584 |
| | | | | | | | 1620 | 1561-1584 |
| Predicted siRNA | 181 | NP_001152619 | Zea mays | 1537 | 1564 | 1591 | | |
| 54566 | | | | | | | 1621 | 837-858 |
| | | | | | | | 1622 | 837-858 |
| | | | | | | | 1623 | 837-858 |
| | | | | | | | 1624 | 837-858 |
| | | NP_001130342 | Zea mays | 1538 | 1565 | 1592 | | |
| | | | | | | | 1625 | 212-233 |
| | | | | | | | 1626 | 212-233 |
| | | | | | | | 1627 | 212-233 |
| | | | | | | | 1628 | 212-233 |
| | | | | | | | 1629 | 212-233 |
| Predicted siRNA | 182 | ACN33370 | Zea mays | 1539 | 1566 | 1593 | | |
| 54666 | | | | | | | 1630 | 207-228 |
| | | | | | | | 1631 | 207-228 |
| | | | | | | | 1632 | 207-228 |
| | | | | | | | 1633 | 207-228 |
| | | | | | | | 1634 | 207-228 |
| | | ACR38139 | Zea mays | 1540 | 1567 | 1594 | | |
| | | | | | | | 1635 | 680-701 |
| | | | | | | | 1636 | 680-701 |
| | | | | | | | 1637 | 680-701 |
| | | NP_001141527 | Zea mays | 1541 | 1568 | 1595 | | |
| | | | | | | | 1638 | 848-869 |
| | | | | | | | 1639 | 848-869 |
| | | | | | | | 1640 | 848-869 |
| | | | | | | | 1641 | 848-869 |
| | | | | | | | 1642 | 848-869 |
| | | NP_001130841 | Zea mays | 1542 | 1569 | 1596 | | |
| | | | | | | | 1643 | 327-348 |
| | | | | | | | 1644 | 327-348 |
| | | | | | | | 1645 | 327-348 |
| | | | | | | | 1646 | 327-348 |
| | | | | | | | 1647 | 327-348 |
| | | NP_001183778 | Zea mays | 1543 | 1570 | 1597 | | |
| | | | | | | | 1648 | 588-609 |
| | | | | | | | 1649 | 588-609 |
| | | | | | | | 1650 | 588-609 |
| | | | | | | | 1651 | 588-609 |
| | | | | | | | 1652 | 588-609 |
| Predicted siRNA | 183 | NP_001150152 | Zea mays | 1544 | 1571 | 1598 | | |
| 55684 | | | | | | | 1653 | 231-252 |
| | | | | | | | 1654 | 231-252 |
| | | | | | | | 1655 | 231-252 |
| | | | | | | | 1656 | 231-252 |
| | | | | | | | 1657 | 231-252 |
| | | NP_001146149 | Zea mays | 1545 | 1572 | 1599 | | |
| | | | | | | | 1658 | 252-273 |
| | | | | | | | 1659 | 252-273 |
| | | | | | | | 1660 | 252-273 |
| | | | | | | | 1661 | 252-273 |
| | | | | | | | 1662 | 252-273 |
| | | NP_001141527 | Zea mays | 1546 | 1573 | 1600 | | |
| | | | | | | | 1663 | 846-867 |
| | | | | | | | 1664 | 846-867 |
| | | | | | | | 1665 | 846-867 |
| | | | | | | | 1666 | 846-867 |
| | | | | | | | 1667 | 846-867 |
| Predicted siRNA | 190 | NP_001105185 | Zea mays | 1547 | 1574 | 1601 | | |
| 56658 | | | | | | | 1668 | 102-125 |
| | | | | | | | 1669 | 102-125 |
| | | | | | | | 1670 | 102-125 |
| | | | | | | | 1671 | 102-125 |
| | | | | | | | 1672 | 102-125 |
| Predicted siRNA | 192 | NP_001147862 | Zea mays | 1548 | 1575 | 1602 | | |
| 56885 | | | | | | | 1673 | 1287-1308 |
| | | | | | | | 1674 | 1287-1308 |
| | | | | | | | 1675 | 1287-1308 |
| | | | | | | | 1676 | 1287-1308 |
| | | | | | | | 1677 | 1287-1308 |
| | | XP_002467897 | Sorghum bicolor | 1549 | 1576 | 1603 | | |
| | | | | | | | 1678 | 188-209 |
| | | | | | | | 1679 | 188-209 |
| | | | | | | | 1680 | 188-209 |
| | | | | | | | 1681 | 188-209 |
| | | | | | | | 1682 | 188-209 |
| | | NP_001062056 | Oryza sativa | 1550 | 1577 | 1604 | | |
| | | | Japonica Group | | | | 1683 | 1145-1166 |
| | | | | | | | 1684 | 1145-1166 |
| | | | | | | | 1685 | 1145-1166 |
| | | | | | | | 1686 | 1145-1166 |
| | | | | | | | 1687 | 1145-1166 |
| Predicted siRNA | 203 | NP_001131832 | Zea mays | 1551 | 1578 | 1605 | | |
| 60742 | | | | | | | 1688 | 542-563 |
| | | | | | | | 1689 | 542-563 |
| | | | | | | | 1690 | 542-563 |
| | | | | | | | 1691 | 542-563 |
| | | | | | | | 1692 | 542-563 |
| Predicted siRNA | 80 | NP_001145763 | Zea mays | 1552 | 1579 | 1606 | | |
| 60833 | | | | | | | 1693 | 434-451 |
| | | | | | | | 1694 | 434-451 |
| | | | | | | | 1695 | 434-451 |
| | | | | | | | 1696 | 434-451 |
| | | | | | | | 1697 | 434-451 |
| | | NP_001149387 | Zea mays | 1553 | 1580 | 1607 | | |
| | | | | | | | 1698 | 673-690 |
| | | | | | | | 1699 | 673-690 |
| | | | | | | | 1700 | 673-690 |
| | | | | | | | 1701 | 673-690 |
| | | | | | | | 1702 | 673-690 |
| Predicted zma | 207 | XP_002465048 | Sorghum bicolor | 1554 | 1581 | 1608 | | |
| mir 48482 | | | | | | | 1703 | 380-401 |
| | | | | | | | 1704 | 380-401 |
| | | | | | | | 1705 | 380-401 |
| | | | | | | | 1706 | 380-401 |
| | | | | | | | 1707 | 380-401 |
| Predicted zma | 210 | BAJ84854 | Hordeum vulgare | 1555 | 1582 | 1609 | | |
| mir49259 | | | subsp. vulgare | | | | 1708 | 456-477 |
| | | | | | | | 1709 | 456-477 |
| | | | | | | | 1710 | 456-477 |
| | | | | | | | 1711 | 456-477 |
| | | | | | | | 1712 | 456-477 |
| Predicted zma | 211 | XP_002448827 | Sorghum bicolor | 1556 | 1583 | 1610 | | |
| mir 49642 | | | | | | | 1713 | 129-150 |
| | | ACN25803 | Zea mays | 1557 | 1584 | 1611 | | |
| | | | | | | | 1714 | 1138-1159 |
| | | | | | | | 1715 | 1138-1159 |
| | | | | | | | 1716 | 1138-1159 |
| | | | | | | | 1717 | 1138-1159 |
| | | | | | | | 1718 | 1138-1159 |
| | | ACO72994 | Zea mays | 1558 | 1585 | 1612 | | |
| | | | | | | | 1719 | 174-195 |
| | | | | | | | 1720 | 174-195 |
| | | | | | | | 1721 | 174-195 |
| | | | | | | | 1722 | 174-195 |
| | | | | | | | 1723 | 174-195 |
| Predicted zma | 213 | XP_002452943 | Sorghum bicolor | 1559 | 1586 | 1613 | | |
| mir 50085 | | | | | | | 1724 | 820-840 |
| | | | | | | | 1725 | 820-840 |
| | | | | | | | 1726 | 820-840 |
| | | | | | | | 1727 | 820-840 |
| | | | | | | | 1728 | 820-840 |
| | | NP_001150320 | Zea mays | 1560 | 1587 | 1614 | | |
| | | | | | | | 1729 | 349-369 |
| | | | | | | | 1730 | 349-369 |
| | | XP_002436903 | Sorghum bicolor | 1561 | 1588 | 1615 | | |
| | | | | | | | 1731 | 813-833 |
| | | | | | | | 1732 | 813-833 |
| | | | | | | | 1733 | 813-833 |
| | | | | | | | 1734 | 813-833 |
| | | | | | | | 1735 | 813-833 |
| Predicted zma | 215 | ACN35534 | Zea mays | 1562 | 1589 | 1616 | | |
| mir 50486 | | | | | | | 1736 | 352-373 |
| | | | | | | | 1737 | 352-373 |
| | | | | | | | 1738 | 352-373 |
| | | | | | | | 1739 | 352-373 |

TABLE 17
Target Mimic Examples for miRNAs which were
Downregulated under Drought Stress.

| | | Bulge in |
|---|---|---|
| | | Target Binding |
| | Mir Sequence/ | Sequence/ |
| Mir Name | SEQ ID NO: | SEQ ID NO: |
| Predicted folded 24-nts-long seq | 1 | 1741 |
| 52255 | | |
| Predicted siRNA 55937 | 2 | 1742 |
| Predicted siRNA 56066 | 18 | 1743 |
| Predicted siRNA 58170 | 3 | 1744 |
| Predicted zma mir 47934 | 4 | 1745 |
| Predicted zma mir 48120 | 5 | 1746 |
| Predicted zma mir 48408 | 6 | 1747 |
| Predicted zma mir 48451 | 7 | 1748 |
| Predicted zma mir 48462 | 8 | 1749 |
| Predicted zma mir 48520 | 9 | 1750 |
| Predicted zma mir 48653 | 26 | 1751 |
| Predicted zma mir 48669 | 10 | 1752 |
| Predicted zma mir 48682 | 11 | 1753 |
| Predicted zma mir 48841 | 12 | 1754 |
| Predicted zma mir 48966 | 13 | 1755 |
| Predicted zma mir 49156 | 14 | 1756 |
| Predicted zma mir 49199 | 15 | 1757 |

TABLE 18
Target Mimic Examples for miRNAs which
were Downregulated during Salt Stress.

| | | Bulge in |
|---|---|---|
| | | Target Binding |
| | Mir Sequence/ | Sequence/ |
| Mir Name | SEQ ID NO: | SEQ ID NO: |
| Predicted folded 24-nts-long seq | 33 | 1758 |
| 54187 | | |
| Predicted zma mir 47990 | 34 | 1759 |
| Predicted zma mir 48459 | 35 | 1760 |
| Predicted zma mir 48753 | 36 | 1761 |
| Predicted zma mir 48824 | 37 | 1762 |
| Predicted zma mir 48848 | 38 | 1763 |
| Predicted zma mir 49575 | 39 | 1764 |
| Predicted zma mir 49817 | 40 | 1765 |
| Predicted zma mir 49855 | 41 | 1766 |
| Predicted zma mir 49862 | 42 | 1767 |

TABLE 19
Target Mimic Examples for miRNAs which
were Downregulated during Heat Shock

| | | Bulge in |
|---|---|---|
| | | Target Binding |
| | Mir Sequence/ | Sequence/ |
| Mir Name | SEQ ID NO: | SEQ ID NO: |
| Predicted folded 24-nts-long seq | 53 | 1768 |
| 52724 | | |
| Predicted folded 24-nts-long seq | 54 | 1769 |
| 53866 | | |
| Predicted siRNA 54735 | 55 | 1770 |
| Predicted siRNA 55208 | 56 | 1771 |
| Predicted siRNA 56396 | 57 | 1772 |
| Predicted siRNA 56791 | 58 | 1773 |
| Predicted siRNA 57689 | 59 | 1774 |
| Predicted siRNA 58108 | 60 | 1775 |
| Predicted siRNA 58158 | 61 | 1776 |
| Predicted siRNA 58720 | 62 | 1777 |
| Predicted siRNA 58740 | 63 | 1778 |
| Predicted siRNA 59056 | 64 | 1779 |
| Predicted siRNA 59211 | 65 | 1780 |
| Predicted siRNA 59300 | 66 | 1781 |
| Predicted siRNA 59379 | 67 | 1782 |
| Predicted siRNA 59410 | 68 | 1783 |
| Predicted siRNA 59474 | 69 | 1784 |
| Predicted siRNA 59736 | 70 | 1785 |
| Predicted siRNA 59799 | 71 | 1786 |
| Predicted siRNA 59800 | 72 | 1787 |
| Predicted siRNA 59820 | 73 | 1788 |
| Predicted siRNA 59851 | 74 | 1789 |
| Predicted siRNA 59918 | 75 | 1790 |
| Predicted siRNA 59935 | 76 | 1791 |
| Predicted siRNA 59937 | 77 | 1792 |
| Predicted siRNA 60421 | 78 | 1793 |
| Predicted siRNA 60533 | 79 | 1794 |
| Predicted siRNA 60833 | 80 | 1795 |
| Predicted siRNA 61212 | 81 | 1796 |
| Predicted siRNA 61236 | 82 | 1797 |
| Predicted zma mir 48327 | 83 | 1798 |
| Predicted zma mir 48489 | 84 | 1799 |
| Predicted zma mir 49248 | 85 | 1800 |
| Predicted zma mir 49310 | 86 | 1801 |
| Predicted zma mir 49952 | 87 | 1802 |
| Predicted zma mir 50120 | 88 | 1803 |
| Predicted zma mir 50166 | 89 | 1804 |
| Predicted zma mir 50256 | 90 | 1805 |
| Predicted zma mir 50289 | 91 | 1806 |
| Predicted zma mir 50388 | 92 | 1807 |
| Predicted zma mir 50453 | 93 | 1808 |
| Predicted zma mir 50480 | 94 | 1809 |
| Predicted zma mir 50481 | 95 | 1810 |
| Predicted zma mir 50483 | 96 | 1811 |
| Predicted zma mir 50570 | 97 | 1812 |
| Predicted zma mir 50682 | 98 | 1813 |
| Predicted zma mir 50695 | 99 | 1814 |
| Predicted zma mir 50701 | 100 | 1815 |

### Example 11

### Target Gene Identification Using Bioinformatic Tools

Homologous or orthologous genes to the genes of interest in maize and/or Arabidopsis are found through a proprietary tool that analyzes publicly available genomic as well as expression and gene annotation databases from multiple plant species. Homologous and orthologous protein and nucleotide sequences of target genes of the small RNA sequences of the invention, were found using BLAST having at least 70% identity on at least 60% of the entire master gene length, and are summarized in Table 6-8 below.

TABLE 20
Targets of small RNAs listed in Tables 1 and 2 above

| | Mir | Homolog | Nucleotide | | | | | |
|---|---|---|---|---|---|---|---|---|
| Mir | Binding | NCBI | NCBI GI | | | | Protein | Nucleotide |
| name | Position | Accession | number | Annotation | Identity | Organism | seq id no: | seq id no: |
| ath-miR164c | 758-778 | CAH56057 | 60098047 | hypothetical protein [Zea mays] | 1 | Zea mays | 1816 | 2015 |
| | | XP_002441978 | 242083105 | hypothetical protein SORBIDRAFT_08g006330 | 0.793296 | Sorghum bicolor | 1817 | 2016 |
| | | | | [Sorghum bicolor] &gt:gi\|241942671\|gb\|EES15816.1\| | | | | |
| | | | | hypothetical protein SORBIDRAFT_08g006330 | | | | |
| | | | | [Sorghum bicolor] | | | | |
| | | EAZ00836 | 54362548 | hypothetical protein OsI_22867 [Oryza sativa Indica | 0.701117 | Oryza sativa | 1818 | |
| | | | | Group] | | Indica Group | | |
| | | NP_001057578 | 115467957 | Os06g0344900 [Oryza sativa Japonica Group] | 0.703911 | Oryza sativa | 1819 | 2017 |
| | | | | &gt:gi\|54291113\|dbj\|BAD61787.1\| putative NAM | | Japonica Group | | |
| | | | | [Oryza sativa Japonica Group] | | | | |
| | | | | &gt:gi\|113595618\|dbj\|BAF19492.1\| Os06g0344900 | | | | |
| | | | | [Oryza sativa Japonica Group] | | | | |
| | 671-691 | ADX60129 | 323388648 | NAC transcription factor [Zea mays] | 1 | Zea mays | 1820 | 2018 |
| | | ACF86180 | 194704191 | unknown [Zea mays] | 1 | Zea mays | 1821 | 2019 |
| | | NP_001148231 | 226507011 | NAC domain-containing protein 21/22 [Zea mays] | 0.956364 | Zea mays | 1822 | 2020 |
| | | | | &gt:gi\|195616832\|gb\|ACG30246.1\| NAC domain- | | | | |
| | | | | containing protein 21/22 [Zea mays] | | | | |
| | | XP_002443999 | 242078460 | hypothetical protein SORBIDRAFT_07g005610 | 0.854545 | Sorghum bicolor | 1823 | 2021 |
| | | | | [Sorghum bicolor] &gt:gi\|241940349\|gb\|EES13494.1\| | | | | |
| | | | | hypothetical protein SORBIDRAFT_07g005610 | | | | |
| | | | | [Sorghum bicolor] | | | | |
| | 409-429 | NP_00116879 | 293334660 | hypothetical protein LOC100382594 [Zea mays] | 1 | Zea mays | 1824 | 2022 |
| | | | | &gt:gi\|223973065\|gb\|ACN30720.1\| unknown [Zea mays] | | | | |
| | | XP_002463116 | 242050743 | hypothetical protein SORBIDRAFT_02g038150 | 0.944532 | Sorghum bicolor | 1825 | 2023 |
| | | | | [Sorghum bicolor] &gt:gi\|241926493\|gb\|EER99637.1\| | | | | |
| | | | | hypothetical protein SORBIDRAFT_02g038150 | | | | |
| | | | | [Sorghum bicolor] | | | | |
| | | NP_001060161 | 115473124 | Os07g0592200 [Oryza sativa Japonica Group] | 0.852615 | Oryza sativa | 1826 | 2024 |
| | | | | &gt:gi\|29027762\|dbj\|BAC65898.1\| putative CND41, | | Japonica Group | | |
| | | | | chloroplast nucleoid DNA binding protein | | | | |
| | | | | [Oryza sativa Japonica Group] | | | | |
| | | | | &gt:gi\|113611697\|dbj\|BAF22075.1\| Os07g0592200 | | | | |
| | | | | [Oryza sativa Japonica Group] | | | | |
| | | EEE67511 | 54398660 | hypothetical protein OsJ_24961 [Oryza sativa Japonica | 0.852615 | Oryza sativa | 1827 | |
| | | | | Group] | | Japonica Group | | |
| | | EEC82371 | 54362548 | hypothetical protein OsI_26705 [Oryza sativa Indica | 0.85103 | Oryza sativa | 1828 | |
| | | | | Group] | | Indica Group | | |
| | | BAK03251 | 326511103 | predicted protein [Hordeum vulgare subsp. vulgare] | 0.806656 | Hordeum vulgare | 1829 | 2025 |
| | | | | | | subsp. vulgare | | |
| | | BAK02500 | 326501421 | predicted protein [Hordeum vulgare subsp. vulgare] | 0.805071 | Hordeum vulgare | 1830 | 2026 |
| | | | | | | subsp. vulgare | | |
| | 854-874 | ACG42560 | 195646183 | CUC2 [Zea mays] | 1 | Zea mays | 1831 | 2027 |
| | | XP_002452517 | 242062455 | hypothetical protein SORBIDRAFT_04g027290 | 0.843902 | Sorghum bicolor | 1832 | 2028 |
| | | | | [Sorghum bicolor] &gt:gi\|241932348\|gb\|EES05493.1\| | | | | |
| | | | | hypothetical protein SORBIDRAFT_04g027290 | | | | |
| | | | | [Sorghum bicolor] | | | | |
| | 953-973 | ACN28126 | 223948084 | unknown [Zea mays] | 1 | Zea mays | 1833 | 2029 |
| | | NP_001132469 | 212723361 | hypothetical protein LOC100193925 [Zea mays] | 0.922652 | Zea mays | 1834 | 2030 |
| | | | | &gt:gi\|194694468\|gb\|ACF81318.1\| unknown [Zea mays] | | | | |
| | | ACG34790 | 195625919 | hypothetical protein [Zea mays] | 0.911602 | Zea mays | 1835 | 2031 |
| | | XP_002455261 | 242052230 | hypothetical protein SORBIDRAFT_03g007360 | 0.737569 | Sorghum bicolor | 1836 | 2032 |
| | | | | [Sorghum bicolor] &gt:gi\|241927236\|gb\|EES00381.1\| | | | | |
| | | | | hypothetical protein SORBIDRAFT_03g007360 | | | | |
| | | | | [Sorghum bicolor] | | | | |
| osa-miR2907a | 205-226 | XP_002448756 | 242077639 | hypothetical protein SORBIDRAFT_06g032630 | 1 | Sorghum bicolor | 1837 | 2033 |
| | | | | [Sorghum bicolor] &gt:gi\|241939939\|gb\|EES13084.1\| | | | | |
| | | | | hypothetical protein SORBIDRAFT_06g032630 | | | | |
| | | | | [Sorghum bicolor] | | | | |
| | | NP_001143260 | 226499897 | hypothetical protein LOC100275789 [Zea mays] | 0.797468 | Zea mays | 1838 | 2034 |
| | | | | &gt:gi\|195616738\|gb\|ACG30199.1\| hypothetical | | | | |
| | | | | protein [Zea mays] | | | | |
| | | NP_001143008 | 226500337 | hypothetical protein LOC100275471 [Zea mays] | 0.794304 | Zea mays | 1839 | 2035 |
| | | | | &gt:gi\|195612812\|gb\|ACG28236.1\| hypothetical | | | | |
| | | | | protein [Zea mays] | | | | |
| | 797-818 | XP_002458417 | 242058542 | hypothetical protein SORBIDRAFT_03g033100 | 1 | Sorghum bicolor | 1840 | 2036 |
| | | | | [Sorghum bicolor] &gt:gi\|241930392\|gb\|EES03537.1\| | | | | |
| | | | | hypothetical protein SORBIDRAFT_03g033100 | | | | |
| | | | | [Sorghum bicolor] | | | | |
| | | NP_001151156 | 226529416 | LOC100284789 [Zea mays] | 0.852518 | Zea mays | 1841 | 2037 |
| | | | | &gt:gi\|195644686\|gb\|ACG41811.1\| phosphatase | | | | |
| | | | | phospho1 [Zea mays] | | | | |
| | | ACG25081 | 195606501 | phosphatase phospho1 [Zea mays] | 0.848921 | Zea mays | 1842 | 2038 |
| | | NP_001130133 | 212275705 | hypothetical protein LOC100191227 [Zea mays] | 0.845324 | Zea mays | 1843 | 2039 |
| | | | | &gt:gi\|194688368\|gb\|ACF78268.1\| unknown [Zea mays] | | | | |
| | | | | &gt:gi\|195606422\|gb\|ACG25041.1\| phosphatase | | | | |
| | | | | phospho1 [Zea mays] | | | | |
| | | | | &gt:gi\|195606828\|gb\|ACG25244.1\| phosphatase | | | | |
| | | | | phospho1 [Zea mays] | | | | |
| | | BAJ95704 | 326507253 | predicted protein [Hordeum vulgare subsp. vulgare] | 0.78777 | Hordeum vulgare | 1844 | 2040 |
| | | | | | | subsp. vulgare | | |
| | | BAJ88911 | 326525728 | predicted protein [Hordeum vulgare subsp. vulgare] | 0.784173 | Hordeum vulgare | 1845 | 2041 |
| | | | | | | subsp. vulgare | | |
| | | EAY75640 | 54362548 | hypothetical protein OsI_03545 [Oryza sativa Indica | 0.780576 | Oryza sativa | 1846 | |
| | | | | Group] | | Indica Group | | |
| | | ABI95993 | 115361862 | acid phosphatase [Oryza sativa Japonica Group] | 0.773381 | Oryza sativa | 1847 | 2042 |
| | | | | | | Japonica Group | | |
| | | NP_001044089 | 115439618 | Os01g0720400 [Oryza sativa Japonica Group] | 0.773381 | Oryza sativa | 1848 | 2043 |
| | | | | &gt:gi\|18461219\|dbj\|BAB84416.1\| phosphatase-like | | Japonica Group | | |
| | | | | [Oryza sativa Japonica Group] | | | | |
| | | | | &gt:gi\|113533620\|dbj\|BAF06003.1\| Os01g0720400 | | | | |
| | | | | [Oryza sativa Japonica Group] | | | | |
| Predicted | 264-285 | XP_002462124 | 242048755 | hypothetical protein SORBIDRAFT_02g019550 | 1 | Sorghum bicolor | 1849 | 2044 |
| siRNA 55507 | | | | [Sorghum bicolor] &gt:gi\|241925501\|gb\|EER98645.1\| | | | | |
| | | | | hypothetical protein SORBIDRAFT_02g019550 | | | | |
| | | | | [Sorghum bicolor] | | | | |
| | | NP_001132708 | 212720661 | hypothetical protein LOC100194191 [Zea mays] | 0.880952 | Zea mays | 1850 | 2045 |
| | | | | &gt:gi\|194695164\|gb\|ACF81666.1\| unknown | | | | |
| | | | | [Zea mays] | | | | |
| Predicted | 399-420 | AAA33496 | 293901 | Zea mI [Zea mays] | 1 | Zea mays | 1851 | 2046 |
| siRNA 55629 | | | | | | | | |
| | | 2HCZ_X | | Chain X, Crystal Structure Of Expb1 (Zea M 1), A | 0.973822 | Zea mays | 1852 | |
| | | | | Beta-Expansin And Group-1 Pollen Allergen From Maize | | | | |
| | 765-786 | NP_001105209 | 162460197 | expansin-B9 precursor [Zea mays] | 1 | Zea mays | 1853 | 2047 |
| | | | | &gt:gi\|115502389\|sp\|Q07154.2\|EXPB9_MAIZE | | | | |
| | | | | RecName: Full = Expansin-B9; AltName: Full = Beta- | | | | |
| | | | | expansin-1b; AltName: Full = Pollen allergen Zea m 1; | | | | |
| | | | | AltName: Full = ZmEXPB9; AltName: Allergen = Zea m | | | | |
| | | | | 1; Flags: Precursor &gt:gi\|28630919\|gb\|AAO45607.1\| | | | | |
| | | | | beta-expansin 9 protein [Zea mays] | | | | |
| | | P58738 | | RecName: Full=Expansin-B1; AltName: Full=Allergen | 0.977695 | Zea mays | 1854 | |
| | | | | Zea m 1d; AltName: Full=Beta-expansin-1a; AltName: | | | | |
| | | | | Full = Pollen allergen Zea m 1; AltName: | | | | |
| | | | | Full = ZmEXPB1; AltName: Allergen = Zea m 1; Flags: | | | | |
| | | | | Precursor &gt:gi\|28630923\|gb\|AAO45608.1\| beta- | | | | |
| | | | | expansin 1 protein [Zea mays] | | | | |
| | | | | &gt:gi\|224035093\|gb\|ACN36622.1\| unknown [Zea mays] | | | | |
| | | AAK56124 | 14193760 | beta-expansin 1 [Zea mays] | 0.973978 | Zea mays | 1855 | 2048 |
| | | ACF86930 | 194705691 | unknown [Zea mays] | 0.97026 | Zea mays | 1856 | 2049 |
| | | ACA23876 | 168419913 | Pas n 1 allergen precursor [Paspalum notatum] | 0.836431 | Paspalum notatum | 1857 | 2050 |
| | | XP_002467539 | 242040288 | hypothetical protein SORBIDRAFT_01g029840 | 0.802974 | Sorghum bicolor | 1858 | 2051 |
| | | | | [Sorghum bicolor] &gt:gi\|241921393\|gb\|EER94537.1\| | | | | |
| | | | | hypothetical protein SORBIDRAFT_01g029840 | | | | |
| | | | | [Sorghum bicolor] | | | | |
| | | NP_001065305 | 115483269 | Os10g0548600 [Oryza sativa Japonica Group] | 0.732342 | Oryza sativa | 1859 | 2052 |
| | | | | &gt:gi\|75232354\|sp\|Q7XCG7.1\|EXPB9_ORYSJ | | Japonica Group | | |
| | | | | RecName: Full = Expansin-B9; AltName: Full = Beta- | | | | |
| | | | | expansin-9; AltName: Full = OsEXPB9; AltName: | | | | |
| | | | | Full = OsaEXPb1.6; Flags: Precursor | | | | |
| | | | | &gt:gi\|8118437\|gb\|AAF72990.1\|AF261277_1 beta- | | | | |
| | | | | expansin [Oryza sativa] | | | | |
| | | | | &gt:gi\|13876539\|gb\|AAK43515.1\|AC020666_25 beta- | | | | |
| | | | | expansin [Oryza sativa Japonica Group] | | | | |
| | | | | &gt:gi\|31433387\|gb\|AAP54906.1\| Beta-expansin 1a | | | | |
| | | | | precursor, putative, expressed [Oryza sativa Japonica | | | | |
| | | | | Group] &gt:gi\|33338561\|gb\|AAQ13902.1\| pollen | | | | |
| | | | | allergen [Oryza sativa] | | | | |
| | | | | &gt:gi\|113639837\|dbj\|BAF27142.1\| Os10g0548600 | | | | |
| | | | | [Oryza sativa Japonica Group] | | | | |
| | | | | &gt:gi\|125575604\|gb\|EAZ16888.1\| hypothetical protein | | | | |
| | | | | OsJ_32365 [Oryza sativa Japonica Group] | | | | |
| | | | | &gt:gi\|215704498\|dbj\|BAG93932.1\| unnamed protein | | | | |
| | | | | product [Oryza sativa Japonica Group] | | | | |
| | | EAY79426 | 54362548 | hypothetical protein OsI_34559 [Oryza sativa Indica | 0.728625 | Oryza sativa | 1860 | |
| | | | | Group] | | Indica Group | | |
| Predicted | 1289-1310 | XP_002445918 | 242082298 | hypothetical protein SORBIDRAFT_07g028020 | 1 | Sorghum bicolor | 1861 | 2053 |
| siRNA 55937 | | | | [Sorghum bicolor] &gt:gi\|241942268\|gb\|EES15413.1\| | | | | |
| | | | | hypothetical protein SORBIDRAFT_07g028020 | | | | |
| | | | | [Sorghum bicolor] | | | | |
| | | AAY89657 | 68303941 | beta 1,2-xylosyltransferase [Zea mays] | 0.926499 | Zea mays | 1862 | 2054 |
| | | NP_001105845 | 162464153 | Beta-1,2-xylosyltransferase [Zea mays] | 0.914894 | Zea mays | 1863 | 2055 |
| | | | | &gt:gi\|83715789\|emb\|CAJ47425.1\| Beta-1,2- | | | | |
| | | | | xylosyltransferase [Zea mays] | | | | |
| | | CAI11448 | 55956967 | beta-2-xylosyltransferase [Saccharum officinarum] | 0.870406 | Saccharum officinarum | 1864 | 2056 |
| | | CAJ47422 | 83715782 | Beta-1,2-xylosyltransferase [Sorghum bicolor] | 0.831721 | Sorghum bicolor | 1865 | 2057 |
| | | NP_001062177 | 115477161 | Os08g0503800 [Oryza sativa Japonica Group] | 0.808511 | Oryza sativa | 1866 | 2058 |
| | | | | &gt:gi\|42408140\|dbj\|BAD09279.1\| putative beta 1,2- | | Japonica Group | | |
| | | | | xylosyltransferase [Oryza sativa Japonica Group] | | | | |
| | | | | &gt:gi\|113624146\|dbj\|BAF24091.1\| Os08g0503800 | | | | |
| | | | | [Oryza sativa Japonica Group] | | | | |
| | | BAJ90540 | 326495835 | predicted protein [Hordeum vulgare subsp. vulgare] | 0.775629 | Hordeum vulgare | 1867 | 2059 |
| | | | | | | subsp. vulgare | | |
| | | CAJ47421 | 83715780 | Beta-1,2-xylosyltransferase [Hordeum vulgare] | 0.783366 | Hordeum vulgare | 1868 | 2060 |
| | 1225-1246 | ACG29798 | 195615935 | beta-2-xylosyltransferase [Zea mays] | 1 | Zea mays | 1869 | 2061 |
| Predicted | 336-356 | XP_002465700 | 242036610 | hypothetical protein SORBIDRAFT_01g044050 | 1 | Sorghum bicolor | 1870 | 2062 |
| siRNA 57283 | | | | [Sorghum bicolor] &gt:gi\|241919554\|gb\|EER92698.1\| | | | | |
| | | | | hypothetical protein SORBIDRAFT_01g044050 | | | | |
| | | | | [Sorghum bicolor] | | | | |
| | | NP_001105083 | 162463855 | serine acetyltransferase2 [Zea mays] | 0.877814 | Zea mays | 1871 | 2063 |
| | | | | &gt:gi\|25991549\|gb\|AAN76865.1\|AF453838_1 satase | | | | |
| | | | | isoform II [Zea mays] | | | | |
| | | EAY88901 | 54362548 | hypothetical protein OsI_10380 [Oryza sativa Indica | 0.794212 | Oryza sativa | 1872 | |
| | | | | Group] | | Indica Group | | |
| | | NP_001049265 | 297600483 | Os03g0196600 [Oryza sativa Japonica Group] | 0.800643 | Oryza sativa | 1873 | 2064 |
| | | | | &gt:gi\|122224506\|sp\|Q10QH1.1\|SAT4_ORYSJ | | Japonica Group | | |
| | | | | RecName: Full = Probable serine acetyltransferase 4; | | | | |
| | | | | AltName: Full = OsSERAT2; 2 | | | | |
| | | | | &gt:gi\|108706662\|gb\|ABF94457.1\| satase isoform II, | | | | |
| | | | | putative, expressed [Oryza sativa Japonica Group] | | | | |
| | | | | &gt:gi\|255674283\|dbj\|BAF11179.2\| Os03g0196600 | | | | |
| | | | | [Oryza sativa Japonica Group] | | | | |
| | | BAJ93017 | 326523692 | predicted protein [Hordeum vulgare subsp. vulgare] | 0.784566 | Hordeum vulgare | 1874 | 2065 |
| | | | | | | subsp. vulgare | | |
| | | NP_001049193 | 297600437 | Os03g0185000 [Oryza sativa Japonica Group] | 0.752412 | Oryza sativa | 1875 | 2066 |
| | | | | &gt:gi\|223635827\|sp\|Q0DUI1.2\|SAT3_ORYSJ | | Japonica Group | | |
| | | | | RecName: Full = Probable serine acetyltransferase 3; | | | | |
| | | | | AltName: Full = OsSERAT2; 1 | | | | |
| | | | | &gt:gi\|108706556\|gb\|ABF94351.1\| satase isoform II, | | | | |
| | | | | putative [Oryza sativa Japonica Group] | | | | |
| | | | | &gt:gi\|255674260\|dbj\|BAF11107.2\| Os03g0185000 | | | | |
| | | | | [Oryza sativa Japonica Group] | | | | |
| | 647-667 | XP_002452644 | 242062709 | hypothetical protein SORBIDRAFT_04g029800 | 1 | Sorghum bicolor | 1876 | 2067 |
| | | | | [Sorghum bicolor] &gt:gi\|241932475\|gb\|EES05620.1\| | | | | |
| | | | | hypothetical protein SORBIDRAFT_04g029800 | | | | |
| | | | | [Sorghum bicolor] | | | | |
| | | ACN27676 | 223947184 | unknown [Zea mays] | 0.979592 | Zea mays | 1877 | 2068 |
| | | NP_001151821 | 226506715 | px19-like protein [Zea mays] | 0.97449 | Zea mays | 1878 | 2069 |
| | | | | &gt:gi\|195649987\|gb\|ACG44461.1\| px19-like protein | | | | |
| | | | | [Zea mays] | | | | |
| | | BAJ98658 | 326498460 | predicted protein [Hordeum vulgare subsp. vulgare] | 0.857143 | Hordeum vulgare | 1879 | 2070 |
| | | | | &gt:gi\|326526295\|dbj\|BAJ97164.1\| predicted protein | | subsp. vulgare | | |
| | | | | [Hordeum vulgare subsp. vulgare] | | | | |
| | | NP_001047936 | 115448312 | Os02g0717900 [Oryza sativa Japonica Group] | 0.862245 | Oryza sativa | 1880 | 2071 |
| | | | | &gt:gi\|45735865\|dbj\|BAD12899.1\| unknown protein | | Japonica Group | | |
| | | | | [Oryza sativa Japonica Group] | | | | |
| | | | | &gt:gi\|113537467\|dbj\|BAF09850.1\| Os02g0717900 | | | | |
| | | | | [Oryza sativa Japonica Group] | | | | |
| | | | | &gt:gi\|125540909\|gb\|EAY87304.1\| hypothetical protein | | | | |
| | | | | OsI_08707 [Oryza sativa Indica Group] | | | | |
| | | | | &gt:gi\|215697545\|dbj\|BAG91539.1\| unnamed protein | | | | |
| | | | | product [Oryza sativa Japonica Group] | | | | |
| | | EAZ24403 | 54398660 | hypothetical protein OsJ_08158 [Oryza sativa Japonica | 0.852041 | Oryza sativa | 1881 | |
| | | | | Group] | | Japonica Group | | |
| | 77-97 | XP_002459575 | 242043407 | hypothetical protein SORBIDRAFT_02g006870 | 1 | Sorghum bicolor | 1882 | 2072 |
| | | | | [Sorghum bicolor] &gt:gi\|241922952\|gb\|EER96096.1\| | | | | |
| | | | | hypothetical protein SORBIDRAFT_02g006870 | | | | |
| | | | | [Sorghum bicolor] | | | | |
| | | CAH39852 | 74473394 | putative protein kinase [Zea mays] | 0.927063 | Zea mays | 1883 | 2073 |
| | | | | &gt:gi\|238015268\|gb\|ACR38669.1\| unknown | | | | |
| | | | | [Zea mays] | | | | |
| | | CAH55606 | 76057830 | putative protein kinase [Zea mays] | 0.915547 | Zea mays | 1884 | 2074 |
| | | NP_001105820 | 162461746 | putative protein kinase [Zea mays] | 0.888676 | Zea mays | 1885 | 2075 |
| | | | | &gt:gi\|74473391\|emb\|CAH39850.1\| putative protein | | | | |
| | | | | kinase [Zea mays] | | | | |
| | | NP_001064960 | 115482733 | Os10g0497600 [Oryza sativa Japonica Group] | 0.882917 | Oryza sativa | 1886 | 2076 |
| | | | | &gt:gi\|78708852\|gb\|ABB47827.1\| Protein kinase | | Japonica Group | | |
| | | | | domain containing protein, expressed [Oryza sativa | | | | |
| | | | | Japonica Group] &gt:gi\|113639569\|dbj\|BAF26874.1\| | | | | |
| | | | | Os10g0497600 [Oryza sativa Japonica Group] | | | | |
| | | | | &gt:gi\|215736900\|dbj\|BAG95829.1\| unnamed protein | | | | |
| | | | | product [Oryza sativa Japonica Group] | | | | |
| | | XP_002440368 | 242089070 | hypothetical protein SORBIDRAFT_09g030440 | 0.879079 | Sorghum bicolor | 1887 | 2077 |
| | | | | [Sorghum bicolor] &gt:gi\|241945653\|gb\|EES18798.1\| | | | | |
| | | | | hypothetical protein SORBIDRAFT_09g030440 | | | | |
| | | | | [Sorghum bicolor] | | | | |
| | | CAH39851 | 74473392 | putative protein kinase [Zea mays] | 0.886756 | Zea mays | 1888 | 2078 |
| | | EEC67247 | 54362548 | hypothetical protein OsI_34186 [Oryza sativa Indica | 0.884837 | Oryza sativa | 1889 | |
| | | | | Group] | | Indica Group | | |
| | | ACR36632 | 238011193 | unknown [Zea mays] | 0.884837 | Zea mays | 1890 | 2079 |
| | | | | &gt:gi\|238013118\|gb\|ACR37594.1\| unknown | | | | |
| | | | | [Zea mays] | | | | |
| | | EEE51214 | 54398660 | hypothetical protein OsJ_32036 [Oryza sativa Japonica | 0.882917 | Oryza sativa | 1891 | |
| | | | | Group] | | Japonica Group | | |
| Predicted | 0-19 | ACG30934 | 195618207 | 60S ribosomal protein L19-3 [Zea mays] | 1 | Zea mays | 1892 | 2080 |
| siRNA 58170 | | | | &gt:gi\|195635187\|gb\|ACG37062.1\| 60S ribosomal | | | | |
| | | | | protein L19-3 [Zea mays] | | | | |
| | | | | &gt:gi\|223973915\|gb\|ACN31145.1\| unknown | | | | |
| | | | | [Zea mays] | | | | |
| | | XP_002453326 | 242064073 | hypothetical protein SORBIDRAFT_04g003890 | 0.961353 | Sorghum bicolor | 1893 | 2081 |
| | | | | [Sorghum bicolor] &gt:gi\|241933157\|gb\|EES06302.1\| | | | | |
| | | | | hypothetical protein SORBIDRAFT_04g003890 | | | | |
| | | | | [Sorghum bicolor] | | | | |
| | | XP_002458037 | 242057782 | hypothetical protein SORBIDRAFT_03g025960 | 0.94686 | Sorghum bicolor | 1894 | 2082 |
| | | | | [Sorghum bicolor] &gt:gi\|241930012\|gb\|EES03157.1\| | | | | |
| | | | | hypothetical protein SORBIDRAFT_03g025960 | | | | |
| | | | | [Sorghum bicolor] | | | | |
| | | NP_001150484 | 226499705 | 60S ribosomal protein L19-3 [Zea mays] | 0.942029 | Zea mays | 1895 | 2083 |
| | | | | &gt:gi\|195639566\|gb\|ACG39251.1\| 60S ribosomal | | | | |
| | | | | protein L19-3 [Zea mays] | | | | |
| | | | | &gt:gi\|238006088\|gb\|ACR34079.1\| unknown | | | | |
| | | | | [Zea mays] | | | | |
| | | ACF82857 | 194697545 | unknown [Zea mays] | 0.932367 | Zea mays | 1896 | 2084 |
| | | NP_001148813 | 226503680 | 60S ribosomal protein L19-3 [Zea mays] | 0.922705 | Zea mays | 1897 | 2085 |
| | | | | &gt:gi\|195622310\|gb\|ACG32985.1\| 60S ribosomal | | | | |
| | | | | protein L19-3 [Zea mays] | | | | |
| | | EAY90757 | 54362548 | hypothetical protein OsI_12360 [Oryza sativa Indica | 0.913043 | Oryza sativa | 1898 | |
| | | | | Group] | | Indica Group | | |
| | | AAP05800 | 29837750 | putative ribosomal protein L19 [Oryza sativa Japonica | 0.903382 | Oryza sativa | 1899 | 2086 |
| | | | | Group] &gt:gi\|50399976\|gb\|AAT76364.1\| putative | | Japonica Group | | |
| | | | | ribosomal protein L19 [Oryza sativa Japonica Group] | | | | |
| | | | | &gt:gi\|108709489\|gb\|ABF97284.1\| 60S ribosomal | | | | |
| | | | | protein L19-3, putative, expressed [Oryza sativa | | | | |
| | | | | Japonica Group] &gt:gi\|125586925\|gb\|EAZ27589.1\| | | | | |
| | | | | hypothetical protein OsJ_11537 [Oryza sativa Japonica | | | | |
| | | | | Group] | | | | |
| | | NP_001050051 | 115452900 | Os03g0337800 [Oryza sativa Japonica Group] | 0.874396 | Oryza sativa | 1900 | 2087 |
| | | | | &gt:gi\|108708037\|gb\|ABF95832.1\| 60S ribosomal | | Japonica Group | | |
| | | | | protein L19-1, putative, expressed [Oryza sativa | | | | |
| | | | | Japonica Group] &gt:gi\|113548522\|dbj\|BAF11965.1\| | | | | |
| | | | | Os03g0337800 [Oryza sativa Japonica Group] | | | | |
| | | | | &gt:gi\|125543790\|gb\|EAY89929.1\| hypothetical protein | | | | |
| | | | | OsI_11477 [Oryza sativa Indica Group] | | | | |
| | | | | &gt:gi\|125586185\|gb\|EAZ26849.1\| hypothetical protein | | | | |
| | | | | OsJ_10765 [Oryza sativa Japonica Group] | | | | |
| | | | | &gt:gi\|215734967\|dbj\|BAG95689.1\| unnamed protein | | | | |
| | | | | product [Oryza sativa Japonica Group] | | | | |
| | | | | &gt:gi\|215765261\|dbj\|BAG86958.1\| unnamed protein | | | | |
| | | | | product [Oryza sativa Japonica Group] | | | | |
| | | NP_001136706 | 219362984 | hypothetical protein LOC100216841 [Zea mays] | 0.884058 | Zea mays | 1901 | 2088 |
| | | | | &gt:gi\|194696712\|gb\|ACF82440.1\| unknown | | | | |
| | | | | [Zea mays] | | | | |
| | 98-117 | EEC83614 | 54362548 | hypothetical protein OsI_29322 [Oryza sativa Indica | 1 | Oryza sativa | 1902 | |
| | | | | Group] | | Indica Group | | |
| | | NP_001061858 | 115476523 | Os08g0430700 [Oryza sativa Japonica Group] | 0.994937 | Oryza sativa | 1903 | 2089 |
| | | | | &gt:gi\|38175475\|dbj\|BAD01172.1\| putative UVB- | | Japonica Group | | |
| | | | | resistance protein UVR8 [Oryza sativa Japonica Group] | | | | |
| | | | | &gt:gi\|113623827\|dbj\|BAF23772.1\| Os08g0430700 | | | | |
| | | | | [Oryza sativa Japonica Group] | | | | |
| | | | | &gt:gi\|215734996\|dbj\|BAG95718.1\| unnamed protein | | | | |
| | | | | product [Oryza sativa Japonica Group] | | | | |
| | | | | &gt:gi\|222640598\|gb\|EEE68730.1\| hypothetical protein | | | | |
| | | | | OsJ_27407 [Oryza sativa Japonica Group] | | | | |
| | | NP_001141854 | 226495690 | hypothetical protein LOC100273996 [Zea mays] | 0.868354 | Zea mays | 1904 | 2090 |
| | | | | &gt:gi\|194706180\|gb\|ACF87174.1\| unknown [Zea mays] | | | | |
| | | BAJ86407 | 326506177 | predicted protein [Hordeum vulgare subsp. vulgare] | 0.868354 | Hordeum vulgare | 1905 | 2091 |
| | | | | | | subsp. vulgare | | |
| | | XP_002283479 | 225451992 | PREDICTED: hypothetical protein [Vitis vinifera] | 0.78481 | Vitis vinifera | 1906 | 2092 |
| | | | | &gt:gi\|296087299\|emb\|CBI33673.3\| unnamed protein | | | | |
| | | | | product [Vitis vinifera] | | | | |
| | | XP_002329325 | 224127629 | predicted protein [Populus trichocarpa] | 0.777215 | Populus trichocarpa | 1907 | 2093 |
| | | | | &gt:gi\|222870779\|gb\|EEF07910.1\| predicted protein | | | | |
| | | | | [Populus trichocarpa] | | | | |
| | | XP_002516745 | 255551397 | Ran GTPase binding protein, putative [Ricinus communis] | 0.767089 | Ricinus communis | 1908 | 2094 |
| | | | | &gt:gi\|223544118\|gb\|EEF45643.1\| Ran | | | | |
| | | | | GTPase binding protein, putative [Ricinus communis] | | | | |
| | | ADE76711 | 294462323 | unknown [Picea sitchensis] | 0.886076 | Picea sitchensis | 1909 | 2095 |
| | | XP_002873752 | 297811736 | regulator of chromosome condensation family protein | 0.764557 | Arabidopsis lyrata | 1910 | 2096 |
| | | | | [Arabidopsis lyrata subsp. lyrata] | | subsp. lyrata | | |
| | | | | &gt:gi\|297319589\|gb\|EFH50011.1\| regulator of | | | | |
| | | | | chromosome condensation family protein | | | | |
| | | | | [Arabidopsis lyrata subsp. lyrata] | | | | |
| | 82-101 | NP_001169682 | 293336964 | hypothetical protein LOC100383563 [Zea mays] | 1 | Zea mays | 1911 | 2097 |
| | | | | &gt:gi\|224030677\|gb\|ACN34414.1\| unknown [Zea mays] | | | | |
| | | | | &gt:gi\|224030857\|gb\|ACN34504.1\| unknown | | | | |
| | | | | [Zea mays] | | | | |
| | | NP_001047275 | 115446990 | Os02g0588500 [Oryza sativa Japonica Group] | 0.741036 | Oryza sativa | 1912 | 2098 |
| | | | | &gt:gi\|46806229\|dbj\|BAD17453.1\| putative GPI- | | Japonica Group | | |
| | | | | anchored protein [Oryza sativa Japonica Group] | | | | |
| | | | | &gt:gi\|113536806\|dbj\|BAF09189.1\| Os02g0588500 | | | | |
| | | | | [Oryza sativa Japonica Group] | | | | |
| | | | | &gt:gi\|125582687\|gb\|EAZ23618.1\| hypothetical protein | | | | |
| | | | | OsJ_07316 [Oryza sativa Japonica Group] | | | | |
| | 190-209 | XP_002452366 | 242062153 | hypothetical protein SORBIDRAFT_04g024440 | 1 | Sorghum bicolor | 1913 | 2099 |
| | | | | [Sorghum bicolor] &gt:gi\|241932197\|gb\|EES05342.1\| | | | | |
| | | | | hypothetical protein SORBIDRAFT_04g024440 | | | | |
| | | | | [Sorghum bicolor] | | | | |
| | 105-124 | NP_001140994 | 226507777 | hypothetical protein LOC100273073 [Zea mays] | 1 | Zea mays | 1914 | 2100 |
| | | | | &gt:gi\|194702086\|gb\|ACF85127.1\| unknown | | | | |
| | | | | [Zea mays] | | | | |
| | | NP_197108 | 186523277 | regulator of chromosome condensation repeat- | 0.77892 | Arabidopsis thaliana | 1915 | 2101 |
| | | | | containing protein [Arabidopsis thaliana] | | | | |
| | | | | &gt:gi\|9755650\|emb\|CAC01803.1\| UVB-resistance | | | | |
| | | | | protein-like [Arabidopsis thaliana] | | | | |
| | | | | &gt:gi\|114213505\|gb\|ABI54335.1\| At5g16040 | | | | |
| | | | | [Arabidopsis thaliana] | | | | |
| | | | | &gt:gi\|332004857\|gb\|AED92240.1\| regulator of | | | | |
| | | | | chromosome condensation repeat-containing protein | | | | |
| | | | | [Arabidopsis thaliana] | | | | |
| Predicted | 804-827 | XP_002451473 | 242060367 | hypothetical protein SORBIDRAFT_04g002490 | 1 | Sorghum bicolor | 1916 | 2102 |
| siRNA 60433 | | | | [Sorghum bicolor] &gt:gi\|241931304\|gb\|EES04449.1\| | | | | |
| | | | | hypothetical protein SORBIDRAFT_04g002490 | | | | |
| | | | | [Sorghum bicolor] | | | | |
| | | NP_001168856 | 293336630 | hypothetical protein LOC100382661 [Zea mays] | 0.875 | Zea mays | 1917 | 2103 |
| | | | | &gt:gi\|223973357\|gb\|ACN30866.1\| unknown | | | | |
| | | | | [Zea mays] | | | | |
| | | EEC72413 | 54362548 | hypothetical protein OsI_05713 [Oryza sativa Indica | 0.810897 | Oryza sativa | 1918 | |
| | | | | Group] &gt:gi\|222622105\|gb\|EEE56237.1\| hypothetical | | Indica Group | | |
| | | | | protein OsJ_05240 [Oryza sativa Japonica Group] | | | | |
| | | NP_001045780 | 115444000 | Os02g0129900 [Oryza sativa Japonica Group] | 0.785256 | Oryza sativa | 1919 | 2104 |
| | | | | &gt:gi\|41053058\|dbj\|BAD07988.1\| hypothetical protein | | Japonica Group | | |
| | | | | [Oryza sativa Japonica Group] | | | | |
| | | | | &gt:gi\|113535311\|dbj\|BAF07694.1\| Os02g0129900 | | | | |
| | | | | [Oryza sativa Japonica Group] | | | | |
| | | BAJ92423 | 326516535 | predicted protein [Hordeum vulgare subsp. vulgare] | 0.791667 | Hordeum vulgare | 1920 | 2105 |
| | | | | &gt:gi\|326523493\|dbj\|BAJ92917.1\| predicted protein | | subsp. vulgare | | |
| | | | | [Hordeum vulgare subsp. vulgare] | | | | |
| Predicted | 840-861 | XP_002456624 | 242054956 | hypothetical protein SORBIDRAFT_03g039630 | 1 | Sorghum bicolor | 1921 | 2106 |
| zma mir | | | | [Sorghum bicolor] &gt:gi\|241928599\|gb\|EES01744.1\| | | | | |
| 47934 | | | | hypothetical protein SORBIDRAFT_03g039630 | | | | |
| | | | | [Sorghum bicolor] | | | | |
| | | NP_001149037 | 226528422 | protein phosphatase 2C [Zea mays] | 0.876214 | Zea mays | 1922 | 2107 |
| | | | | &gt:gi\|195624162\|gb\|ACG33911.1\| protein phosphatase | | | | |
| | | | | 2C [Zea mays] | | | | |
| | | NP_001044788 | 115441016 | Os01g0846300 [Oryza sativa Japonica Group] | 0.742718 | Oryza sativa | 1923 | 2108 |
| | | | | &gt:gi\|75285811\|sp\|Q5N9N2.1\|P2C09_ORYSJ | | Japonica Group | | |
| | | | | RecName: Full = Probable protein phosphatase 2C 9; | | | | |
| | | | | Short = OsPP2C09 &gt:gi\|56784698\|dbj\|BAD81824.1\| | | | | |
| | | | | putative protein phosphatase 2C [Oryza sativa Japonica | | | | |
| | | | | Group] &gt:gi\|113534319\|dbj\|BAF06702.1\| | | | | |
| | | | | Os01g0846300 [Oryza sativa Japonica Group] | | | | |
| | | | | &gt:gi\|218189363\|gb\|EEC71790.1\| hypothetical protein | | | | |
| | | | | OsI_04415 [Oryza sativa Indica Group] | | | | |
| | | | | &gt:gi\|222619530\|gb\|EEE55662.1\| hypothetical protein | | | | |
| | | | | OsJ_04060 [Oryza sativa Japonica Group] | | | | |
| | | BAK01548 | 326489128 | predicted protein [Hordeum vulgare subsp. vulgare] | 0.73301 | Hordeum vulgare | 1924 | 2109 |
| | | | | | | subsp. vulgare | | |
| Predicted | 2885-2905 | AAL76004 | 18568260 | putative gag-pol polyprotein [Zea mays] | 1 | Zea mays | 1925 | |
| zma mir | 2677-2697 | AAN40030 | 23928433 | putative gag-pol polyprotein [Zea mays] | 1 | Zea mays | 1926 | |
| 48043 | 2926-2946 | AAN40035 | 23928433 | putative gag-pol polyprotein [Zea mays] | 1 | Zea mays | 1927 | |
| | 2425-2445 | AAL66760 | 18254408 | putative gag protein [Zea mays] | 1 | Zea mays | 1928 | |
| | 114-134 | AAL75488 | 46200524 | putative pol protein [Zea mays] | 1 | Zea mays | 1929 | |
| | 225-245 | AAD09018 | 4185305 | polyprotein [Zea mays] | 1 | Zea mays | 1930 | 2110 |
| Predicted | 102-122 | NP_001169451 | 293335512 | hypothetical protein LOC100383322 [Zea mays] | 1 | Zea mays | 1931 | 2111 |
| zma mir | | | | &gt:gi\|224029469\|gb\|ACN33810.1\| unknown | | | | |
| 48408 | | | | [Zea mays] | | | | |
| | | EEC68244 | 54362548 | hypothetical protein OsI_36259 [Oryza sativa Indica | 0.856471 | Oryza sativa | 1932 | |
| | | | | Group] | | Indica Group | | |
| | | NP_001067969 | 115485650 | Os11g0514800 [Oryza sativa Japonica Group] | 0.851765 | Oryza sativa | 1933 | 2112 |
| | | | | &gt:gi\|77551091\|gb\|ABA93888.1\| expressed protein | | Japonica Group | | |
| | | | | [Oryza sativa Japonica Group] | | | | |
| | | | | &gt:gi\|113645191\|dbj\|BAF28332.1\| Os11g0514800 | | | | |
| | | | | [Oryza sativa Japonica Group] | | | | |
| | | | | &gt:gi\|125577286\|gb\|EAZ18508.1\| hypothetical protein | | | | |
| | | | | OsJ_34037 [Oryza sativa Japonica Group] | | | | |
| | | | | &gt:gi\|215686713\|dbj\|BAG88966.1\| unnamed protein | | | | |
| | | | | product [Oryza sativa Japonica Group] | | | | |
| | | | | &gt:gi\|215708828\|dbj\|BAG94097.1\| unnamed protein | | | | |
| | | | | product [Oryza sativa Japonica Group] | | | | |
| | 35-55 | XP_002463174 | 242050859 | hypothetical protein SORBIDRAFT_02g039100 | 1 | Sorghum bicolor | 1934 | 2113 |
| | | | | [Sorghum bicolor] &gt:gi\|241926551\|gb\|EER99695.1\| | | | | |
| | | | | hypothetical protein SORBIDRAFT_02g039100 | | | | |
| | | | | [Sorghum bicolor] | | | | |
| | | NP_001150845 | 226504299 | DGCR14 protein [Zea mays] | 0.873727 | Zea mays | 1935 | 2114 |
| | | | | &gt:gi\|195642346\|gb\|ACG40641.1\| DGCR14 protein | | | | |
| | | | | [Zea mays] | | | | |
| | | BAJ99113 | 326502969 | predicted protein [Hordeum vulgare subsp. vulgare] | 0.843177 | Hordeum vulgare | 1936 | 2115 |
| | | | | | | subsp. vulgare | | |
| | | EAZ01087 | 54362548 | hypothetical protein OsI_23114 [Oryza sativa Indica | 0.830957 | Oryza sativa | 1937 | |
| | | | | Group] | | Indica Group | | |
| | | NP_001057711 | 115468223 | Os06g0502900 [Oryza sativa Japonica Group] | 0.835031 | Oryza sativa | 1938 | 2116 |
| | | | | &gt:gi\|52076439\|dbj\|BAD45267.1\| putative ES2 protein | | Japonica Group | | |
| | | | | [Oryza sativa Japonica Group] | | | | |
| | | | | &gt:gi\|113595751\|dbj\|BAF19625.1\| Os06g0502900 | | | | |
| | | | | [Oryza sativa Japonica Group] | | | | |
| | | | | &gt:gi\|215737222\|dbj\|BAG96151.1\| unnamed protein | | | | |
| | | | | product [Oryza sativa Japonica Group] | | | | |
| | | | | &gt:gi\|215767859\|dbj\|BAH00088.1\| unnamed protein | | | | |
| | | | | product [Oryza sativa Japonica Group] | | | | |
| | | EAZ37129 | 54398660 | hypothetical protein OsJ_21470 [Oryza sativa Japonica | 0.775967 | Oryza sativa | 1939 | |
| | | | | Group] | | Japonica Group | | |
| Predicted | 63-84 | NP_001148466 | 226493425 | protein phosphatase 2C [Zea mays] | 1 | Zea mays | 1940 | 2117 |
| zma mir | | | | &gt:gi\|195619560\|gb\|ACG31610.1\| protein phosphatase | | | | |
| 48514 | | | | 2C [Zea mays] | | | | |
| | | NP_001042103 | 115434689 | Os01g0164600 [Oryza sativa Japonica Group] | 0.787234 | Oryza sativa | 1941 | 2118 |
| | | | | &gt:gi\|75164086\|sp\|Q942P9.1\|P2C01_ORYSJ | | Japonica Group | | |
| | | | | RecName: Full = Probable protein phosphatase 2C 1; | | | | |
| | | | | Short = OsPP2C01 &gt:gi\|15528748\|dbj\|BAB64790.1\| | | | | |
| | | | | putative senescence-associated protein [Oryza sativa | | | | |
| | | | | Japonica Group] &gt:gi\|21327992\|dbj\|BAC00581.1\| | | | | |
| | | | | putative senescence-associated protein [Oryza sativa | | | | |
| | | | | Japonica Group] &gt:gi\|113531634\|dbj\|BAF04017.1\| | | | | |
| | | | | Os01g0164600 [Oryza sativa Japonica Group] | | | | |
| | | | | &gt:gi\|125569151\|gb\|EAZ10666.1\| hypothetical protein | | | | |
| | | | | OsJ_00496 [Oryza sativa Japonica Group] | | | | |
| | | EAY72662 | 54362548 | hypothetical protein OsI_00528 [Oryza sativa Indica | 0.787234 | Oryza sativa | 1942 | |
| | | | | Group] | | Indica Group | | |
| | | BAJ94449 | 326494659 | predicted protein [Hordeum vulgare subsp. vulgare] | 0.784195 | Hordeum vulgare | 1943 | 2119 |
| | | | | | | subsp. vulgare | | |
| | 54-75 | XP_002438740 | 242096499 | hypothetical protein SORBIDRAFT_10g025330 | 1 | Sorghum bicolor | 1944 | 2120 |
| | | | | [Sorghum bicolor] &gt:gi\|241916963\|gb\|EER90107.1\| | | | | |
| | | | | hypothetical protein SORBIDRAFT_10g025330 | | | | |
| | | | | [Sorghum bicolor] | | | | |
| | | ACG29270 | 195614879 | amino acid permease [Zea mays] | 0.952479 | Zea mays | 1945 | 2121 |
| | | NP_001130863 | 212274856 | hypothetical protein LOC100191967 [Zea mays] | 0.952479 | Zea mays | 1946 | 2122 |
| | | | | &gt:gi\|194690296\|gb\|ACF79232.1\| unknown [Zea mays] | | | | |
| | | | | &gt:gi\|194707684\|gb\|ACF87926.1\| unknown [Zea mays] | | | | |
| | | | | &gt:gi\|224029673\|gb\|ACN33912.1\| unknown [Zea mays] | | | | |
| | | NP_001058189 | 115469179 | Os06g0644700 [Oryza sativa Japonica Group] | 0.867769 | Oryza sativa | 1947 | 2123 |
| | | | | &gt:gi\|51535520\|dbj\|BAD37439.1\| amino acid | | Japonica | | |
| | | | | transporter-like protein [Oryza sativa Japonica Group] | | Group | | |
| | | | | &gt:gi\|113596229\|dbj\|BAF20103.1\| Os06g0644700 | | | | |
| | | | | [Oryza sativa Japonica Group] | | | | |
| | | EAZ01859 | 54362548 | hypothetical protein OsI_23880 [Oryza sativa Indica | 0.873967 | Oryza sativa | 1948 | |
| | | | | Group] | | Indica Group | | |
| | | BAJ85749 | 326495305 | predicted protein [Hordeum vulgare subsp. vulgare] | 0.845041 | Hordeum vulgare | 1949 | 2124 |
| | | | | | | subsp. vulgare | | |
| | | BAJ95660 | 326507165 | predicted protein [Hordeum vulgare subsp. vulgare] | 0.797521 | Hordeum vulgare | 1950 | 2125 |
| | | | | | | subsp. vulgare | | |
| | | EEE66107 | 54398660 | hypothetical protein OsJ_22140 [Oryza sativa Japonica | 0.743802 | Oryza sativa | 1951 | |
| | | | | Group] | | Japonica Group | | |
| Predicted | 1615-1636 | NP_001130670 | 212275146 | hypothetical protein LOC100191773 [Zea mays] | 1 | Zea mays | 1952 | 2126 |
| zma mir | | | | &gt:gi\|194689790\|gb\|ACF78979.1\| unknown | | | | |
| 48669 | | | | [Zea mays] | | | | |
| | | ACL54094 | 219887438 | unknown [Zea mays] | 0.940397 | Zea mays | 1953 | 2127 |
| | | XP_002438836 | 242096691 | hypothetical protein SORBIDRAFT_10g026920 | 0.763797 | Sorghum bicolor | 1954 | 2128 |
| | | | | [Sorghum bicolor] &gt:gi\|241917059\|gb\|EER90203.1\| | | | | |
| | | | | hypothetical protein SORBIDRAFT_10g026920 | | | | |
| | | | | [Sorghum bicolor] | | | | |
| Predicted | 416-437 | AAL76004 | 18568260 | putative gag-pol polyprotein [Zea mays] | 1 | Zea mays | 1955 | |
| zma mir | 1232-1253 | XP_002449124 | 242067694 | hypothetical protein SORBIDRAFT_05g005470 | 1 | Sorghum bicolor | 1956 | 2129 |
| 48682 | | | | [Sorghum bicolor] &gt:gi\|241934967\|gb\|EES08112.1\| | | | | |
| | | | | hypothetical protein SORBIDRAFT_05g005470 | | | | |
| | | | | [Sorghum bicolor] | | | | |
| | | ACG29168 | 195614675 | aldehyde dehydrogenase, dimeric NADP-preferring | 0.916318 | Zea mays | 1957 | 2130 |
| | | | | [Zea mays] | | | | |
| | | NP_001168661 | 293331192 | hypothetical protein LOC100382449 [Zea mays] | 0.90795 | Zea mays | 1958 | 2131 |
| | | | | &gt:gi\|223950009\|gb\|ACN29088.1\| unknown | | | | |
| | | | | [Zea mays] | | | | |
| | | NP_001148092 | 226509003 | aldehyde dehydrogenase, dimeric NADP-preferring | 0.914226 | Zea mays | 1959 | 2132 |
| | | | | [Zea mays] &gt:gi\|195615734\|gb\|ACG29697.1\| | | | | |
| | | | | aldehyde dehydrogenase, dimeric NADP-preferring | | | | |
| | | | | [Zea mays] | | | | |
| | | ACF85700 | 194703231 | unknown [Zea mays] | 0.912134 | Zea mays | 1960 | 2133 |
| | | NP_001065921 | 115484518 | Os11g0186200 [Oryza sativa Japonica Group] | 0.803347 | Oryza sativa | 1961 | 2134 |
| | | | | &gt:gi\|62954909\|gb\|AAY23278.1\| aldehyde | | Japonica Group | | |
| | | | | dehydrogenase, putative [Oryza sativa Japonica Group] | | | | |
| | | | | &gt:gi\|108864076\|gb\|ABA91775.2\| aldehyde | | | | |
| | | | | dehydrogenase family protein, expressed [Oryza sativa | | | | |
| | | | | Japonica Group] &gt:gi\|113644625\|dbj\|BAF27766.1\| | | | | |
| | | | | Os11g0186200 [Oryza sativa Japonica Group] | | | | |
| | | | | &gt:gi\|215737694\|dbj\|BAG96824.1\| unnamed protein | | | | |
| | | | | product [Oryza sativa Japonica Group] | | | | |
| | | | | &gt:gi\|215737793\|dbj\|BAG96923.1\| unnamed protein | | | | |
| | | | | product [Oryza sativa Japonica Group] | | | | |
| | | | | &gt:gi\|218185391\|gb\|EEC67818.1\| hypothetical protein | | | | |
| | | | | OsI_35395 [Oryza sativa Indica Group] | | | | |
| | | | | &gt:gi\|222615645\|gb\|EEE51777.1\| hypothetical protein | | | | |
| | | | | OsJ_33226 [Oryza sativa Japonica Group] | | | | |
| | | AAX96338 | 45860991 | aldehyde dehydrogenase, putative [Oryza sativa | 0.803347 | Oryza sativa | 1962 | 2135 |
| | | | | Japonica Group] | | Japonica Group | | |
| | | BAK01937 | 326492306 | predicted protein [Hordeum vulgare subsp. vulgare] | 0.763598 | Hordeum vulgare | 1963 | 2136 |
| | | | | | | subsp. vulgare | | |
| Predicted | 1977-1997 | ADU32889 | 315493433 | embryonic flower 1 protein [Eulaliopsis binata] | 1 | Eulaliopsis binata | 1964 | 2137 |
| zma mir | | ABC69154 | 85062576 | EMF-like [Zea mays] | 0.923445 | Zea mays | 1965 | 2138 |
| 50109 | | NP_001105530 | 162461707 | VEF family protein [Zea mays] | 0.934609 | Zea mays | 1966 | 2139 |
| | | | | &gt:gi\|29569111\|gb\|AAO84022.1\| VEF family protein | | | | |
| | | | | [Zea mays] &gt:gi\|60687422\|gb\|AAX35735.1\| | | | | |
| | | | | embryonic flower 2 [Zea mays] | | | | |
| | | ABB77210 | 82469918 | EMF2 [Dendrocalamus latiflorus] | 0.805423 | Dendrocalamus latiflorus | 1967 | 2140 |
| | | AAX78232 | 62275660 | embryonic flower 2 [Triticum aestivum] | 0.797448 | Triticum aestivum | 1968 | 2141 |
| | | NP_001062825 | 115478459 | Os09g0306800 [Oryza sativa Japonica Group] | 0.757576 | Oryza sativa | 1969 | 2142 |
| | | | | &gt:gi\|255678755\|dbj\|BAF24739.2\| Os09g0306800 | | Japonica Group | | |
| | | | | [Oryza sativa Japonica Group] | | | | |
| | | BAD36510 | 51091694 | putative VEF family protein [Oryza sativa Japonica | 0.757576 | Oryza sativa | 1970 | |
| | | | | Group] | | Japonica Group | | |
| | | ADU32890 | 315493435 | embryonic flower 2 protein [Eulaliopsis binata] | 0.757576 | Eulaliopsis binata | 1971 | 2143 |
| | | BAJ99275 | 326503299 | predicted protein [Hordeum vulgare subsp. vulgare] | 0.76874 | Hordeum vulgare | 1972 | 2144 |
| | | | | | | subsp. vulgare | | |
| | | BAD99131 | 66796110 | HvEMF2b [Hordeum vulgare] | 0.770335 | Hordeum vulgare | 1973 | 2145 |
| | 1748-1768 | NP_001105530 | 162461707 | VEF family protein [Zea mays] | 1 | Zea mays | 1974 | 2146 |
| | | | | &gt:gi\|29569111\|gb\|AAO84022.1\| VEF family protein | | | | |
| | | | | [Zea mays] &gt:gi\|60687422\|gb\|AAX35735.1\| | | | | |
| | | | | embryonic flower 2 [Zea mays] | | | | |
| | 580-600 | XP_002454482 | 242066385 | hypothetical protein SORBIDRAFT_04g031920 | 1 | Sorghum bicolor | 1975 | 2147 |
| | | | | [Sorghum bicolor] &gt:gi\|241934313\|gb\|EES07458.1\| | | | | |
| | | | | hypothetical protein SORBIDRAFT_04g031920 | | | | |
| | | | | [Sorghum bicolor] | | | | |
| | | ACN30672 | 223972968 | unknown [Zea mays] | 0.942529 | Zea mays | 1976 | 2148 |
| | | NP_001183461 | 308044322 | hypothetical protein LOC100501893 [Zea mays] | 0.941092 | Zea mays | 1977 | 2149 |
| | | | | &gt:gi\|238011698\|gb\|ACR36884.1\| unknown | | | | |
| | | | | [Zea mays] | | | | |
| | | Q6EPQ3 | | RecName: Full = SPX domain-containing membrane | 0.87069 | Oryza sativa | 1978 | |
| | | | | protein Os02g45520 | | Japonica Group | | |
| | | | | &gt:gi\|306756291\|sp\|A2X8A7.2\|SPXM1_ORYSI | | | | |
| | | | | RecName: Full = SPX domain-containing membrane | | | | |
| | | | | protein OsI_08463 &gt:gi\|50252990\|dbj\|BAD29241.1\| | | | | |
| | | | | SPX (SYG1/Pho81/XPR1) domain-containing protein- | | | | |
| | | | | like [Oryza sativa Japonica Group] | | | | |
| | | | | &gt:gi\|50253121\|dbj\|BAD29367.1\| SPX | | | | |
| | | | | (SYG1/Pho81/XPR1) domain-containing protein-like | | | | |
| | | | | [Oryza sativa Japonica Group] | | | | |
| | | BAJ95234 | 326502341 | predicted protein [Hordeum vulgare subsp. vulgare] | 0.83477 | Hordeum vulgare | 1979 | 2150 |
| | | | | | | subsp. vulgare | | |
| | | CAD41659 | 38605939 | OSJNBa0019K04.6 [Oryza sativa Japonica Group] | 0.808908 | Oryza sativa | 1980 | |
| | | | | &gt:gi\|25591348\|gb\|EAZ31698.1\| hypothetical protein | | Japonica Group | | |
| | | | | OsJ_15847 [Oryza sativa Japonica Group] | | | | |
| | | NP_001053611 | 115460021 | Os04g0573000 [Oryza sativa Japonica Group] | 0.808908 | Oryza sativa | 1981 | 2151 |
| | | | | &gt:gi\|306756012\|sp\|B8AT51.1\|SPXM2_ORYSI | | Japonica Group | | |
| | | | | RecName: Full = SPX domain-containing membrane | | | | |
| | | | | protein OsI_17046 | | | | |
| | | | | &gt:gi\|306756288\|sp\|Q0JAW2.2\|SPXM2_ORYSJ | | | | |
| | | | | RecName: Full = SPX domain-containing membrane | | | | |
| | | | | protein Os04g0573000 | | | | |
| | | | | &gt:gi\|215694614\|dbj\|BAG89805.1\| unnamed protein | | | | |
| | | | | product [Oryza sativa Japonica Group] | | | | |
| | | | | &gt:gi\|218195403\|gb\|EEC77830.1\| hypothetical protein | | | | |
| | | | | OsI_17046 [Oryza sativa Indica Group] | | | | |
| | | | | &gt:gi\|255675707\|dbj\|BAF15525.2\| Os04g0573000 | | | | |
| | | | | [Oryza sativa Japonica Group] | | | | |
| | | CAH66957 | 116309919 | OSIGBa0147H17.5 [Oryza sativa Indica Group] | 0.806034 | Oryza sativa | 1982 | 2152 |
| | | | | | | Indica Group | | |
| | | XP_002446964 | 242074055 | hypothetical protein SORBIDRAFT_06g025950 | 0.784483 | Sorghum bicolor | 1983 | 2153 |
| | | | | [Sorghum bicolor] &gt:gi\|241938147\|gb\|EES11292.1\| | | | | |
| | | | | hypothetical protein SORBIDRAFT_06g025950 | | | | |
| | | | | [Sorghum bicolor] | | | | |
| | | XP_002282540 | 225426756 | PREDICTED: hypothetical protein [Vitis vinifera] | 0.721264 | Vitis vinifera | 1984 | 2154 |
| | | | | &gt:gi\|297742609\|emb\|CBI34758.3\| unnamed protein | | | | |
| | | | | product [Vitis vinifera] | | | | |
| | 965-985 | XP_002462548 | 242049607 | hypothetical protein SORBIDRAFT_02g027920 | 1 | Sorghum bicolor | 1985 | 2155 |
| | | | | [Sorghum bicolor] &gt:gi\|241925925\|gb\|EER99069.1\| | | | | |
| | | | | hypothetical protein SORBIDRAFT_02g027920 | | | | |
| | | | | [Sorghum bicolor] | | | | |
| | | NP_001145770 | 226498793 | hypothetical protein LOC100279277 [Zea mays] | 0.881919 | Zea mays | 1986 | 2156 |
| | | | | &gt:gi\|219884365\|gb\|ACL52557.1\| unknown | | | | |
| | | | | [Zea mays] | | | | |
| | | ACN34477 | 224030802 | unknown [Zea mays] | 0.852399 | Zea mays | 1987 | 2157 |
| | | NP_001145176 | 226530255 | hypothetical protein LOC100278416 [Zea mays] | 0.852399 | Zea mays | 1988 | 2158 |
| | | | | &gt:gi\|195652339\|gb\|ACG45637.1\| hypothetical | | | | |
| | | | | protein [Zea mays] | | | | |
| | 1075-1095 | NP_001130294 | 212274814 | hypothetical protein LOC100191388 [Zea mays] | 1 | Zea mays | 1989 | 2159 |
| | | | | &gt:gi\|194688768\|gb\|ACF78468.1\| unknown | | | | |
| | | | | [Zea mays] | | | | |
| | | NP_001062638 | 115478085 | Os09g0135400 [Oryza sativa Japonica Group] | 0.786982 | Oryza sativa | 1990 | 2160 |
| | | | | &gt:gi\|47848428\|dbj\|BAD22285.1\| putative | | Japonica Group | | |
| | | | | octicosapeptide/Phox/Bem1p (PB1) domain-containing | | | | |
| | | | | protein [Oryza sativa Japonica Group] | | | | |
| | | | | &gt:gi\|113630871\|dbj\|BAF24552.1\| Os09g0135400 | | | | |
| | | | | [Oryza sativa Japonica Group] | | | | |
| sbi-miR164c | 161-181 | AAL83928 | 19070614 | D-type cyclin [Zea mays] | 1 | Zea mays | 1991 | 2161 |
| | | ACL54102 | 219887454 | unknown [Zea mays] | 0.974359 | Zea mays | 1992 | 2162 |
| | | XP_002444532 | 242079526 | hypothetical protein SORBIDRAFT_07g023350 | 0.720513 | Sorghum bicolor | 1993 | 2163 |
| | | | | [Sorghum bicolor] &gt:gi\|241940882\|gb\|EES14027.1\| | | | | |
| | | | | hypothetical protein SORBIDRAFT_07g023350 | | | | |
| | | | | [Sorghum bicolor] | | | | |
| | | NP_001152238 | 226502938 | cyclin delta-2 [Zea mays] | 0.702564 | Zea mays | 1994 | 2164 |
| | | | | &gt:gi\|195654177\|gb\|ACG46556.1\| | | | | |
| | | | | cyclin delta-2 [Zea mays] | | | | |
| | 467-487 | ACN31118 | 223973860 | unknown [Zea mays] | 1 | Zea mays | 1995 | 2165 |
| | 347-367 | XP_002460244 | 242044745 | hypothetical protein SORBIDRAFT_02g025260 | 1 | Sorghum bicolor | 1996 | 2166 |
| | | | | [Sorghum bicolor] &gt:gi\|241923621\|gb\|EER96765.1\| | | | | |
| | | | | hypothetical protein SORBIDRAFT_02g025260 | | | | |
| | | | | [Sorghum bicolor] | | | | |
| | | ACG44940 | 195650944 | uncharacterized ACR [Zea mays] | 0.904145 | Zea mays | 1997 | 2167 |
| | | NP_001130182 | 212275603 | hypothetical protein LOC100191276 [Zea mays] | 0.904145 | Zea mays | 1998 | 2168 |
| | | | | &gt:gi\|194688486\|gb\|ACF78327.1\| unknown | | | | |
| | | | | [Zea mays] | | | | |
| | | BAK00654 | 326524541 | predicted protein [Hordeum vulgare subsp. vulgare] | 0.740933 | Hordeum vulgare | 1999 | 2169 |
| | | | | | | subsp. vulgare | | |
| | | BAJ99672 | 326512633 | predicted protein [Hordeum vulgare subsp. vulgare] | 0.738342 | Hordeum vulgare | 2000 | 2170 |
| | | | | | | subsp. vulgare | | |
| | | NP_001063233 | 115479278 | Os09g0428900 [Oryza sativa Japonica Group] | 0.725389 | Oryza sativa | 2001 | 2171 |
| | | | | &gt:gi\|50726497\|dbj\|BAD34105.1\| VirR/VirH-like | | Japonica Group | | |
| | | | | protein [Oryza sativa Japonica Group] | | | | |
| | | | | &gt:gi\|113631466\|dbj\|BAF25147.1\| Os09g0428900 | | | | |
| | | | | [Oryza sativa Japonica Group] | | | | |
| | | | | &gt:gi\|215704829\|dbj\|BAG94857.1\| unnamed protein | | | | |
| | | | | product [Oryza sativa Japonica Group] | | | | |
| | | EEE69749 | 54398660 | hypothetical protein OsJ_29445 | 0.725389 | Oryza sativa | 2002 | |
| | | | | [Oryza sativa Japonica Group] | | Japonica Group | | |
| | | EEC84614 | 54362548 | hypothetical protein OsI_31450 [Oryza sativa Indica | 0.735751 | Oryza sativa | 2003 | |
| | | | | Group] | | Indica Group | | |
| | 357-377 | ACN25505 | 223942842 | unknown [Zea mays] | 1 | Zea mays | 2004 | 2172 |
| | | NP_001147770 | 226503046 | NAC domain protein NAC5 [Zea mays] | 0.979885 | Zea mays | 2005 | 2173 |
| | | | | &gt:gi\|195613638\|gb\|ACG28649.1\| NAC domain | | | | |
| | | | | protein NAC5 [Zea mays] | | | | |
| | | XP_002452340 | 242062101 | hypothetical protein SORBIDRAFT_04g023990 | 0.83046 | Sorghum bicolor | 2006 | 2174 |
| | | | | [Sorghum bicolor] &gt:gi\|241932171\|gb\|EES05316.1\| | | | | |
| | | | | hypothetical protein SORBIDRAFT_04g023990 | | | | |
| | | | | [Sorghum bicolor] | | | | |
| | 450-470 | NP_001130182 | 212275603 | hypothetical protein LOC100191276 [Zea mays] | 1 | Zea mays | 2007 | 2175 |
| | | | | &gt:gi\|194688486\|gb\|ACF78327.1\| unknown [Zea | | | | |
| | | | | mays] | | | | |
| | | XP_002460244 | 242044745 | hypothetical protein SORBIDRAFT_02g025260 | 0.838095 | Sorghum bicolor | 2008 | 2176 |
| | | | | [Sorghum bicolor] &gt:gi\|241923621\|gb\|EER96765.1\| | | | | |
| | | | | hypothetical protein SORBIDRAFT_02g025260 | | | | |
| | | | | [Sorghum bicolor] | | | | |
| | 320-340 | ACF87609 | 194707049 | unknown [Zea mays] | 1 | Zea mays | 2009 | 2177 |

TABLE 21
Targets of small RNAs listed in Tables 3 and 4 above

| | Mir | Homolog | Nucleotide | | | | | |
|---|---|---|---|---|---|---|---|---|
| Mir | Binding | NCBI | NCBI | | | | Protein | Nucleotide |
| name | Position | Accession | GI number | Annotation | Identity | Organism | seq id no: | seq id no: |
| Predicted | 1161-1184 | NP_001169815 | 293337028 | hypothetical protein LOC100383707 [Zea mays] | 1 | Zea mays | 2183 | 2356 |
| folded | | | | &gt:gi\|224031809\|gb\|ACN34980.1\| unknown [Zea mays] | | | | |
| 24-nts-long | | | | | | | | |
| seq 54187 | | | | | | | | |
| Predicted | 448-469 | XP_002456346 | 242054400 | hypothetical protein SORBIDRAFT_03g034380 | 1 | Sorghum | 2184 | 2357 |
| siRNA | | | | [Sorghum bicolor] &gt:gi\|241928321\|gb\|EES01466.1\| | | bicolor | | |
| 54895 | | | | hypothetical protein SORBIDRAFT_03g034380 | | | | |
| | | | | [Sorghum bicolor] | | | | |
| | | NP_001168171 | 293333303 | hypothetical protein LOC100381924 [Zea mays] | 0.948753 | Zea mays | 2185 | 2358 |
| | | | | &gt:gi\|223946473\|gb\|ACN27320.1\| unknown [Zea mays] | | | | |
| | | BAK02879 | 326504983 | predicted protein [Hordeum vulgare subsp. vulgare] | 0.858726 | Hordeum | 2186 | 2359 |
| | | | | | | vulgare | | |
| | | | | | | subsp. | | |
| | | | | | | vulgare | | |
| | | NP_001044228 | 115439896 | Os01g0746200 [Oryza sativa Japonica Group] | 0.855956 | Oryza | 2187 | 2360 |
| | | | | &gt:gi\|21902047\|dbj\|BAC05596.1\| putative | | sativa | | |
| | | | | nucleoporin Nup75 [Oryza sativa Japonica Group] | | Japonica | | |
| | | | | &gt:gi\|13533759\|dbj\|BAF06142.1\|Os01g0746200 | | Group | | |
| | | | | [Oryza sativa Japonica Group] | | | | |
| | | EEE55373 | 54398660 | hypothetical protein OsJ_03432 [Oryza sativa Japonica | 0.822715 | Oryza | 2188 | |
| | | | | Group] | | sativa | | |
| | | | | | | Japonica | | |
| | | | | | | Group | | |
| | | BAD87530 | 21902027 | putative nucleoporin Nup75 [Oryza sativa Japonica | 0.709141 | Oryza | 2189 | |
| | | | | Group] | | sativa | | |
| | | | | | | Japonica | | |
| | | | | | | Group | | |
| | | EEC71467 | 54362548 | hypothetical protein OsI_03713 [Oryza sativa Indica | 0.732687 | Oryza | 2190 | |
| | | | | Group] | | sativa | | |
| | | | | | | Indica | | |
| | | | | | | Group | | |
| Predicted | 207-227 | NP_001169431 | 293335380 | hypothetical protein LOC100383302 [Zea mays] | 1 | Zea mays | 2191 | 2361 |
| siRNA | | | | &gt:gi\|224029331\|gb\|ACN33741.1\| unknown [Zea mays] | | | | |
| 55242 | | | | | | | | |
| Predicted | 1392-1415 | XP_002468341 | 242041892 | hypothelical protein SORBIDRAFT_01g044140 | 1 | Sorghum | 2192 | 2362 |
| siRNA | | | | [Sorghum bicolor] &gt:gi\|241922195\|gb\|EER95339.1\| | | bicolor | | |
| 55246 | | | | hypothetical protein SORBIDRAFT_01g044140 | | | | |
| | | | | [Sorghum bicolor] | | | | |
| | | NP_001151876 | 226503834 | membrane protein [Zea mays] | 0.81 | Zea mays | 2193 | 2363 |
| | | | | &gt:gi\|195650527\|gb\|ACG44731.1\| membrane protein | | | | |
| | | | | [Zea mays] | | | | |
| | | ACR36360 | 238010649 | unknown [Zea mays] | 0.81137 | Zea mays | 2194 | 2364 |
| | | NP_001148205 | 226506697 | membrane protein [Zea mays] | 0.857881 | Zea mays | 2195 | 2365 |
| | | | | &gt:gi\|195616684\|gb\|ACG30172.1\| membrane protein | | | | |
| | | | | [Zea mays] | | | | |
| | 1356-1379 | ACN34728 | 224031304 | unknown [Zea mays] | 1 | Zea mays | 2196 | 2366 |
| Predicted | 18-38 | XP_002463627 | 242032464 | hypothetical protein SORBIDRAFT_01g003230 | 1 | Sorghum | 2197 | 2367 |
| siRNA | | | | [Sorghum bicolor] &gt:gi\|241917481\|gb\|EER90625.1\| | | bicolor | | |
| 55402 | | | | hypothetical protein SORBIDRAFT_01g003230 | | | | |
| | | | | [Sorghum bicolor] | | | | |
| | | NP_001151779 | 226493505 | CRAL/TRIO domain containing protein [Zea mays] | 0.93038 | Zea mays | 2198 | 2368 |
| | | | | &gt:gi\|95649633\|gb\|ACG44284.1\| CRAL/TRIO | | | | |
| | | | | domain containing protein [Zea mays] | | | | |
| | | | | &gt:gi\|223945249\|gb\|ACN26708.1\| unknown [Zea mays] | | | | |
| | | | | &gt:gi\|224028997\|gb\|ACN33574.1\| unknown | | | | |
| | | | | [Zea mays] | | | | |
| | | NP_001151588 | 226492275 | CRAL/TRIO domain containing protein [Zea mays] | 0.92827 | Zea mays | 2199 | 2369 |
| | | | | &gt:gi\|195647970\|gb\|ACG43453.1\| CRAL/TRIO | | | | |
| | | | | domain containing protein [Zea mays] | | | | |
| | | ACN25417 | 223942666 | unknown [Zea mays] | 0.93038 | Zea mays | 2200 | 2370 |
| | | | | &gt:gi\|223946125\|gb\|ACN27146.1\| unknown [Zea mays] | | | | |
| | | NP_001051723 | 115456244 | Os03g0820900 [Oryza sativa Japonica Group] | 0.78481 | Oryza | 2201 | 2371 |
| | | | | &gt:gi\|29124131\|gb\|AAO65872.1\| unknown protein | | sativa | | |
| | | | | [Oryza sativa Japonica Group] | | Japonica | | |
| | | | | &gt:gi\|108711795\|gb\|ABF99590.1\| CRAL/TRIO | | Group | | |
| | | | | domain containing protein, expressed | | | | |
| | | | | [Oryza sativa Japonica Group] | | | | |
| | | | | &gt:gi\|108711796\|gb\|ABF99591.1\| | | | | |
| | | | | CRAL/TRIO domain containing protein, expressed | | | | |
| | | | | [Oryza sativa Japonica Group] | | | | |
| | | | | &gt:gi\|113550194\|dbj\|BAF13637.1\| Os03g0820900 | | | | |
| | | | | [Oryza sativa Japonica Group] | | | | |
| | | | | &gt:gi\|215704136\|dbj\|BAG92976.1\| unnamed protein | | | | |
| | | | | product [Oryza sativa Japonica Group] | | | | |
| | | BAK03907 | 326519964 | predicted protein [Hordeum vulgare subsp. vulgare] | 0.759494 | Hordeum | 2202 | 2372 |
| | | | | | | vulgare | | |
| | | | | | | subsp. | | |
| | | | | | | vulgare | | |
| Predicted | 2164-2185 | XP_002453411 | 242064243 | hypothetical protein SORBIDRAFT_04g005580 | 1 | Sorghum | 2203 | 2373 |
| siRNA | | | | [Sorghum bicolor] &gt:gi\|241933242\|gb\|EES06387.1\| | | bicolor | | |
| 56314 | | | | hypothetical protein SORBIDRAFT_04g005580 | | | | |
| | | | | [Sorghum bicolor] | | | | |
| | | NP_001104863 | 162460461 | response regulator 9 [Zea mays] | 0.939778 | Zea mays | 2204 | 2374 |
| | | | | &gt:gi\|14189890\|dbj\|BAB55874.1\| response regulator | | | | |
| | | | | 9 [Zea mays] | | | | |
| | | ADX60157 | 323388704 | ARR-B transcription factor [Zea mays] | 0.939778 | Zea mays | 2205 | 2375 |
| | | NP_001046097 | 115444634 | Os02g0182100 [Oryza sativa Japonica Group] | 0.743265 | Oryza | 2206 | 2376 |
| | | | | &gt:gi\|49388028\|dbj\|BAD25144.1\| putative response | | sativa | | |
| | | | | regulator 9 [Oryza sativa Japonica Group] | | Japonica | | |
| | | | | &gt:gi\|113535628\|dbj\|BAF08011.1\| Os02g0182100 | | Group | | |
| | | | | [Oryza sativa Japonica Group] | | | | |
| | | | | &gt:gi\|118790764\|tpd\|FAA00257.1\| TPA: response | | | | |
| | | | | regulator [Oryza sativa Japonica Group] | | | | |
| | | | | &gt:gi\|215713506\|dbj\|BAG94643.1\| unnamed protein | | | | |
| | | | | product [Oryza sativa Japonica Group] | | | | |
| | | | | &gt:gi\|222622313\|gb\|EEE56445.1\| hypothetical protein | | | | |
| | | | | OsJ_05635 [Oryza sativa Japonica Group] | | | | |
| | | EAY84742 | 54362548 | hypothetical protein OsI_06112 [Oryza sativa Indica | 0.740095 | Oryza | 2207 | |
| | | | | Group] | | sativa | | |
| | | | | | | Indica | | |
| | | | | | | Group | | |
| | | BAJ94963 | 326500593 | predicted protein [Hordeum vulgare subsp. vulgare] | 0.719493 | Hordeum | 2208 | 2377 |
| | | | | | | vulgare | | |
| | | | | | | subsp. | | |
| | | | | | | vulgare | | |
| | 366-387 | XP_002463544 | 242032298 | hypothetical protein SORBIDRAFT_01g001720 | 1 | Sorghum | 2209 | 2378 |
| | | | | [Sorghum bicolor] &gt:gi\|241917398\|gb\|EER90542.1\| | | bicolor | | |
| | | | | hypothetical protein SORBIDRAFT_01g001720 | | | | |
| | | | | [Sorghum bicolor] | | | | |
| | | NP_001146073 | 226508205 | hypothetical protein LOC100279605 [Zea mays] | 0.916342 | Zea mays | 2210 | 2379 |
| | | | | &gt:gi\|219885573\|gb\|ACL53161.1\| unknown [Zea mays] | | | | |
| | | AAX96076 | 45430072 | diphthamide biosynthesis protein 2 [Oryza sativa | 0.774319 | Oryza | 2211 | |
| | | | | Japonica Group] &gt:gi\|77549704\|gb\|ABA92501.1\| | | sativa | | |
| | | | | diphthamide biosynthesis protein 2 containing protein, | | Japonica | | |
| | | | | expressed [Oryza sativa Japonica Group] | | Group | | |
| | | | | &gt:gi\|125576808\|gb\|EAZ18030.1\| hypothetical | | | | |
| | | | | protein OsJ_33577 [Oryza sativa Japonica Group] | | | | |
| | | NP_001067661 | 297611609 | Os11g0265600 [Oryza sativa Japonica Group] | 0.762646 | Oryza | 2212 | 2380 |
| | | | | &gt:gi\|255679981\|dbj\|BAF28024.2\| Os11g0265600 | | sativa | | |
| | | | | [Oryza sativa Japonica Group] | | Japonica | | |
| | | | | | | Group | | |
| | | BAJ96431 | 326516877 | predicted protein [Hordeum vulgare subsp. vulgare] | 0.743191 | Hordeum | 2213 | 2381 |
| | | | | | | vulgare | | |
| | | | | | | subsp. | | |
| | | | | | | vulgare | | |
| Predicted | 836-853 | NP_001044108 | 115439656 | Os01g0723600 [Oryza sativa Japonica Group] | 1 | Oryza | 2214 | 2382 |
| siRNA | | | | &gt:gi\|68571765\|sp\|Q8S2E5.1\|KPRS3_ORYSJ | | sativa | | |
| 58212 | | | | RecName: Full = Ribose-phosphate pyrophosphokinase | | Japonica | | |
| | | | | 3, chloroplastic; AltName: Full = Phosphoribosyl | | Group | | |
| | | | | pyrophosphate synthase 3; Flags: Precursor | | | | |
| | | | | &gt:gi\|20160549\|dbj\|BAB89498.1\| putative | | | | |
| | | | | phosphoribosyl pyrophosphate synthase [Oryza sativa | | | | |
| | | | | Japonica Group] &gt:gi\|113533639\|dbj\|BAF06022.1\| | | | | |
| | | | | Os01g0723600 [Oryza sativa Japonica Group] | | | | |
| | | | | &gt:gi\|215766650\|dbj\|BAG98878.1\| unnamed protein | | | | |
| | | | | product [Oryza sativa Japonica Group] | | | | |
| | | EAY75668 | 54362548 | hypothetical protein OsI_03574 [Oryza sativa Indica | 0.9511 | Oryza | 2215 | |
| | | | | Group] | | sativa Indica | | |
| | | | | | | Group | | |
| | | XP_002456270 | 242054248 | hypothetical protein SORBIDRAFT_03g033270 | 0.843521 | Sorghum | 2216 | 2383 |
| | | | | [Sorghum bicolor] &gt:gi\|241928245\|gb\|EES01390.1\| | | bicolor | | |
| | | | | hypothetical protein SORBIDRAFT_03g033270 | | | | |
| | | | | [Sorghum bicolor] | | | | |
| | | EAZ13389 | 54398660 | hypothetical protein OsJ_03307 [Oryza sativa Japonica | 0.784841 | Oryza | 2217 | |
| | | | | Group] | | sativa | | |
| | | | | | | Japonica | | |
| | | | | | | Group | | |
| | | BAJ93948 | 326488558 | predicted protein [Hordeum vulgare subsp. vulgare] | 0.770171 | Hordeum | 2218 | 2384 |
| | | | | | | vulgare | | |
| | | | | | | subsp. | | |
| | | | | | | vulgare | | |
| | | NP_001144250 | 226496474 | hypothetical protein LOC100277120 [Zea mays] | 0.721271 | Zea mays | 2219 | 2385 |
| | | | | &gt:gi\|95639012\|gb\|ACG38974.1\| hypothetical | | | | |
| | | | | protein [Zea mays] | | | | |
| | 174-191 | XP_002439476 | 242087286 | hypothetical protein SORBIDRAFT_09g007410 | 1 | Sorghum | 2220 | 2386 |
| | | | | [Sorghum bicolor] &gt:gi\|241944761\|gb\|EES17906.1\| | | bicolor | | |
| | | | | hypothetical protein SORBIDRAFT_09g007410 | | | | |
| | | | | [Sorghum bicolor] | | | | |
| | | NP_001130714 | 212274518 | hypothetical protein LOC100191818 [Zea mays] | 0.974522 | Zea mays | 2221 | 2387 |
| | | | | &gt:gi\|195621524\|gb\|ACG32592.1\| ubiquitin- | | | | |
| | | | | conjugating enzyme E2 16 [Zea mays] | | | | |
| | | | | &gt:gi\|195638874\|gb\|ACG38905.1\| ubiquitin- | | | | |
| | | | | conjugating enzyme E2 16 [Zea mays] | | | | |
| | | | | &gt:gi\|224035185\|gb\|ACN36668.1\| unknown [Zea mays] | | | | |
| | | ACF79042 | 194689915 | unknown [Zea mays] | 0.968153 | Zea mays | 2222 | 2388 |
| | | | | &gt:gi\|224033341\|gb\|ACN35746.1\| unknown [Zea mays] | | | | |
| | | NP_001047515 | 115447470 | Os02g0634800 [Oryza sativa Japonica Group] | 0.961783 | Oryza | 2223 | 2389 |
| | | | | &gt:gi\|49387540\|dbj\|BAD25096.1\| putative ubiquitin- | | sativa | | |
| | | | | conjugating enzyme E2 [Oryza sativa Japonica Group] | | Japonica | | |
| | | | | &gt:gi\|49388188\|dbj\|BAD25314.1\| putative ubiquitin- | | Group | | |
| | | | | conjugating enzyme E2 [Oryza sativa Japonica Group] | | | | |
| | | | | &gt:gi\|113537046\|dbj\|BAF09429.1\| Os02g0634800 | | | | |
| | | | | [Oryza sativa Japonica Group] | | | | |
| | | | | &gt:gi\|215765381\|dbj\|BAG87078.1\| unnamed protein | | | | |
| | | | | product [Oryza sativa Japonica Group] | | | | |
| | | NP_001150370 | 226529098 | ubiquitin-conjugating enzyme E2 16 [Zea mays] | 0.961783 | Zea mays | 2224 | 2390 |
| | | | | &gt:gi\|195638722\|gb\|ACG38829.1\| ubiquitin- | | | | |
| | | | | conjugating enzyme E2 16 [Zea mays] | | | | |
| | | | | &gt:gi\|223944475\|gb\|ACN26321.1\| unknown [Zea | | | | |
| | | | | mays] | | | | |
| | | XP_002284068 | 225429994 | PREDICTED: hypothetical protein isoform 2 [Vitis vinifera] | 0.942675 | Vitis | 2225 | 2391 |
| | | | | &gt:gi\|225429997\|ref\|XP_002284062.1\| | | vinifera | | |
| | | | | PREDICTED: hypothetical protein isoform 1 [Vitis vinifera] | | | | |
| | | | | &gt:gi\|296081873\|emb\|CBI20878.3\| unnamed | | | | |
| | | | | protein product [Vitis vinifera] | | | | |
| | | ADV04064 | 317159578 | protein binding/ubiquitin-protein ligase 4 [Hevea brasiliensis] | 0.923567 | Hevea | 2226 | 2392 |
| | | | | | | brasiliensis | | |
| | | ACA24139 | 168472726 | ubiquitin-conjugating enzyme [Lolium temulentum] | 0.936306 | Lolium | 2227 | 2393 |
| | | | | | | temulentum | | |
| | | XP_002872181 | 297808594 | predicted protein [Arabidopsis lyrata subsp. lyrata] | 0.929936 | Arabidopsis | 2228 | 2394 |
| | | | | &gt:gi\|297836036\|ref\|XP_002885900.1\| hypothetical | | lyrata | | |
| | | | | protein ARALYDRAFT_899617 [Arabidopsis lyrata | | subsp. | | |
| | | | | subsp. lyrata] &gt:gi\|297318018\|gb\|EFH48440.1\| | | lyrata | | |
| | | | | predicted protein [Arabidopsis lyrata subsp. lyrata] | | | | |
| | | | | &gt:gi\|297331740\|gb\|EFH62159.1\| hypothetical | | | | |
| | | | | protein ARALYDRAFT_899617 [Arabidopsis lyrata | | | | |
| | | | | subsp. lyrata] | | | | |
| | | NP_568476 | 145358439 | putative ubiquitin-conjugating enzyme E2 21 | 0.929936 | Arabidopsis | 2229 | 2395 |
| | | | | [Arabidopsis thaliana] | | thaliana | | |
| | | | | &gt:gi\|79328676\|ref\|NP_001031939.1\| putative | | | | |
| | | | | ubiquitin-conjugating enzyme E2 21 [Arabidopsis thaliana] | | | | |
| | | | | &gt:gi\|75330089\|sp\|Q8LGF7.1\|PEX4_ARATH | | | | |
| | | | | RecName: Full = Protein PEROXIN-4; Short = AtPEX4; | | | | |
| | | | | AltName: Full = Probable ubiquitin-conjugating enzyme | | | | |
| | | | | E2 21; AltName: Full = Ubiquitin carrier protein 21 | | | | |
| | | | | &gt:gi\|21536556\|gb\|AAM60888.1\| E2, ubiquitin- | | | | |
| | | | | conjugating enzyme, putative [Arabidopsis thaliana] | | | | |
| | | | | &gt:gi\|66354452\|gb\|AAY44861.1\| ubiquitinating | | | | |
| | | | | enzyme [Arabidopsis thaliana] | | | | |
| | | | | &gt:gi\|98961101\|gb\|ABF59034.1\| At5g25760 | | | | |
| | | | | [Arabidopsis thaliana] | | | | |
| | | | | &gt:gi\|332006099\|gb\|AED93482.1\| putative ubiquitin- | | | | |
| | | | | conjugating enzyme E2 21 [Arabidopsis thaliana] | | | | |
| | | | | &gt:gi\|332006100\|gb\|AED93483.1\| putative ubiquitin- | | | | |
| | | | | conjugating enzyme E2 21 [Arabidopsis thaliana] | | | | |
| | 53-70 | XP_002443820 | 242078102 | hypothetical protein SORBIDRAFT_07g002770 | 1 | Sorghum | 2230 | 2396 |
| | | | | [Sorghum bicolor] | | bicolor | | |
| | | | | &gt:gi\|18481702\|gb\|AAL73524.1\|AF466200_3 | | | | |
| | | | | tryptophan synthase beta-subunit [Sorghum bicolor] | | | | |
| | | | | &gt:gi\|241940170\|gb\|EES13315.1\| hypothetical protein | | | | |
| | | | | SORBIDRAFT_07g002770 [Sorghum bicolor] | | | | |
| | | P43284 | | RecName: Full = Tryptophan synthase beta chain 2, | 0.834034 | Zea mays | 2231 | |
| | | | | chloroplastic; AltName: Full = Orange pericarp 2; Flags: | | | | |
| | | | | Precursor &gt:gi\|168574\|gb\|AAA33491.1\| tryptophan | | | | |
| | | | | synthase beta-subunit [Zea mays] | | | | |
| | | EAZ05512 | 54362548 | hypothetical protein OsI_27728 [Oryza sativa Indica | 0.848739 | Oryza | 2232 | |
| | | | | Group] | | sativa | | |
| | | | | | | Indica | | |
| | | | | | | Group | | |
| | | P43283 | | RecName: Full = Tryptophan synthase beta chain 1; | 0.802521 | Zea mays | 2233 | |
| | | | | AltName: Full = Orange pericarp 1 | | | | |
| | | | | &gt:gi\|168572\|gb\|AAA33490.1\| tryptophan synthase | | | | |
| | | | | beta-subunit [Zea mays] | | | | |
| | | ADZ04637 | 325260807 | hypothetical protein [Oryza glaberrima] | 0.848739 | Oryza | 2234 | 2397 |
| | | | | | | glaberrima | | |
| | | XP_002281484 | 225461049 | PREDICTED: hypothetical protein [Vitis vinifera] | 0.752101 | Vitis | 2235 | 2398 |
| | | | | | | vinifera | | |
| | | CBI23954 | 270256979 | unnamed protein product [Vitis vinifera] | 0.752101 | Vitis | 2236 | 2399 |
| | | | | | | vinifera | | |
| | | NP_200292 | 145359253 | tryptophan synthase beta chain [Arabidopsis thaliana] | 0.754202 | Arabidopsis | 2237 | 2400 |
| | | | | &gt:gi\|136251\|sp\|P14671.1\|TRPB1_ARATH | | thaliana | | |
| | | | | RecName: Full = Tryptophan synthase beta chain 1, | | | | |
| | | | | chloroplastic; Flags: Precursor | | | | |
| | | | | &gt:gi\|14194117\|gb\|AAK56253.1\|AF367264_1 | | | | |
| | | | | AT5g54810/MBG8_7 [Arabidopsis thaliana] | | | | |
| | | | | &gt:gi\|166892\|gb\|AAA32878.1\| tryptophan synthase | | | | |
| | | | | beta subunit [Arabidopsis thaliana] | | | | |
| | | | | &gt:gi\|9758261\|dbj\|BAB08760.1\| tryptophan synthase | | | | |
| | | | | beta chain 1 precursor [Arabidopsis thaliana] | | | | |
| | | | | &gt:gi\|21592983\|gb\|AAM64932.1\| tryptophan synthase | | | | |
| | | | | beta chain 1 precursor [Arabidopsis thaliana] | | | | |
| | | | | &gt:gi\|22137210\|gb\|AAM91450.1\| | | | | |
| | | | | AT5g54810/MBG8_7 [Arabidopsis thaliana] | | | | |
| | | | | &gt:gi\|110742593\|dbj\|BAE99210.1\| tryptophan | | | | |
| | | | | synthase beta chain 1 precursor [Arabidopsis thaliana] | | | | |
| | | | | &gt:gi\|332009160\|gb\|AED96543.1\| tryptophan | | | | |
| | | | | synthase beta chain [Arabidopsis thaliana] | | | | |
| | | BAD83779 | 57157620 | tryptophan synthase beta subunit [Polygonum tinctorium] | 0.747899 | Polygonum | 2238 | 2401 |
| | | | | | | tinctorium | | |
| | 1275-1292 | ACF84664 | 194701159 | unknown [Zea mays] | 1 | Zea mays | 2239 | 2402 |
| | | NP_001132166 | 239049858 | hypothetical protein LOC100193588 [Zea mays] | 0.961303 | Zea mays | 2240 | 2403 |
| | | | | &gt:gi\|238908670\|gb\|ACF80898.2\| unknown [Zea mays] | | | | |
| | | XP_002463588 | 242032386 | hypothetical protein SORBIDRAFT_01g002540 | 0.847251 | Sorghum | 2241 | 2404 |
| | | | | [Sorghum bicolor] &gt:gi\|241917442\|gb\|EER90586.1\| | | bicolor | | |
| | | | | hypothetical protein SORBIDRAFT_01g002540 | | | | |
| | | | | [Sorghum bicolor] | | | | |
| | | NP_001142611 | 226501615 | hypothetical protein LOC100274879 [Zea mays] | 0.796334 | Zea mays | 2242 | 2405 |
| | | | | &gt:gi\|195607300\|gb\|ACG25480.1\| hypothetical | | | | |
| | | | | protein [Zea mays] | | | | |
| | 193-210 | ADX60181 | 323388752 | NLP transcription factor [Zea mays] | 1 | Zea mays | 2243 | 2406 |
| | | NP_001151037 | 226532035 | RWP-RK domain containing protein [Zea mays] | 0.996979 | Zea mays | 2244 | 2407 |
| | | | | &gt:gi\|195643826\|gb\|ACG41381.1\| RWP-RK domain | | | | |
| | | | | containing protein [Zea mays] | | | | |
| | | ACF82353 | 194696537 | unknown [Zea mays] | 0.996979 | Zea mays | 2245 | 2408 |
| | | XP_002448339 | 242076805 | hypothetical protein SORBIDRAFT_06g025420 | 0.912387 | Sorghum | 2246 | 2409 |
| | | | | [Sorghum bicolor] &gt:gi\|241939522\|gb\|EES12667.1\| | | bicolor | | |
| | | | | hypothetical protein SORBIDRAFT_06g025420 | | | | |
| | | | | [Sorghum bicolor] | | | | |
| | 191-208 | XP_002442705 | 242084559 | hypothetical protein SORBIDRAFT_08g001540 | 1 | Sorghum | 2247 | 2410 |
| | | | | [Sorghum bicolor] &gt:gi\|241943398\|gb\|EES16543.1\| | | bicolor | | |
| | | | | hypothetical protein SORBIDRAFT_08g001540 | | | | |
| | | | | [Sorghum bicolor] | | | | |
| | | ACN27961 | 223947754 | unknown [Zea mays] | 0.954268 | Zea mays | 2248 | 2411 |
| | | | | &gt:gi\|223950179\|gb\|ACN29173.1\| unknown [Zea mays] | | | | |
| | | | | &gt:gi\|224030475\|gb\|ACN34313.1\| unknown | | | | |
| | | | | [Zea mays] | | | | |
| | | ACN33779 | 224029406 | unknown [Zea mays] | 0.882622 | Zea mays | 2249 | 2412 |
| | | NP_001148977 | 226508337 | LOC100282597 [Zea mays] | 0.881098 | Zea mays | 2250 | 2413 |
| | | | | &gt:gi\|195623744\|gb\|ACG33702.1\| alpha-N- | | | | |
| | | | | arabinofuranosidase A precursor [Zea mays] | | | | |
| | | EEC67598 | 54362548 | hypothetical protein OsI_34967 [Oryza sativa Indica | 0.890244 | Oryza | 2251 | |
| | | | | Group] | | sativa | | |
| | | | | | | Indica | | |
| | | | | | | Group | | |
| | | NP_001065660 | 115483996 | Os11g0131900 [Oryza sativa Japonica Group] | 0.890244 | Oryza | 2252 | 2414 |
| | | | | &gt:gi\|108863956\|gb\|ABA91355.2\| Alpha-L- | | sativa | | |
| | | | | arabinofuranosidase C-terminus family protein, | | Japonica | | |
| | | | | expressed [Oryza sativa Japonica Group] | | Group | | |
| | | | | &gt:gi\|108863957\|gb\|ABG22345.1\| Alpha-L- | | | | |
| | | | | arabinofuranosidase C-terminus family protein, | | | | |
| | | | | expressed [Oryza sativa Japonica Group] | | | | |
| | | | | &gt:gi\|108863958\|gb\|ABG22346.1\| Alpha-L- | | | | |
| | | | | arabinofuranosidase C-terminus family protein, | | | | |
| | | | | expressed [Oryza sativa Japonica Group] | | | | |
| | | | | &gt:gi\|13644364\|dbj\|BAF27505.1\| Os11g0131900 | | | | |
| | | | | [Oryza sativa Japonica Group] | | | | |
| | | | | &gt:gi\|215694468\|dbj\|BAG89431.1\| unnamed protein | | | | |
| | | | | product [Oryza sativa Japonica Group] | | | | |
| | | | | &gt:gi\|222615454\|gb\|EEE51586.1\| hypothetical protein | | | | |
| | | | | OsJ_32826 [Oryza sativa Japonica Group] | | | | |
| | | AAK21880 | 13398413 | arabinoxylan arabinofuranohydrolase isoenzyme | 0.873476 | Hordeum | 2253 | 2415 |
| | | | | AXAH-II [Hordeum vulgare] | | vulgare | | |
| | | NP_001066062 | 115487149 | Os12g0128700 [Oryza sativa Japonica Group] | 0.875 | Oryza | 2254 | 2416 |
| | | | | &gt:gi\|77553575\|gb\|ABA96371.1\| Alpha-L- | | sativa | | |
| | | | | arabinofuranosidase C-terminus family protein, | | Japonica | | |
| | | | | expressed [Oryza sativa Japonica Group] | | Group | | |
| | | | | &gt:gi\|108862132\|gb\|ABA96370.2\| Alpha-L | | | | |
| | | | | arabinofuranosidase C-terminus family protein, | | | | |
| | | | | expressed [Oryza sativa Japonica Group] | | | | |
| | | | | &gt:gi\|113648569\|dbj\|BAF29081.1\| Os12g0128700 | | | | |
| | | | | [Oryza sativa Japonica Group] | | | | |
| | | EEC68793 | 54362548 | hypothetical protein OsI_37345 [Oryza sativa Indica | 0.875 | Oryza | 2255 | |
| | | | | Group] | | sativa | | |
| | | | | | | Indica | | |
| | | | | | | Group | | |
| | | EEE52698 | 54398660 | hypothetical protein OsJ_35098 [Oryza sativa Japonica | 0.855183 | Oryza | 2256 | |
| | | | | Group] | | sativa | | |
| | | | | | | Japonica | | |
| | | | | | | Group | | |
| | 39-56 | XP_002460589 | 242045435 | hypothetical protein SORBIDRAFT_02g031360 | 1 | Sorghum | 2257 | 2417 |
| | | | | [Sorghum bicolor] | | bicolor | | |
| | | | | &gt:gi\|241923966\|gb\|EER97110.1\| | | | | |
| | | | | hypothetical protein SORBIDRAFT_02g031360 | | | | |
| | | | | [Sorghum bicolor] | | | | |
| | | NP_001170202 | 293335825 | hypothetical protein LOC100384152 [Zea mays] | 0.932534 | Zea mays | 2258 | 2418 |
| | | | | &gt:gi\|74011495\|ref\|XP_548544.2\| PREDICTED: | | | | |
| | | | | similar to solute carrier family 15, member 4 [Canis | | | | |
| | | | | familiaris] &gt:gi\|224034265\|gb\|ACN36208.1\| | | | | |
| | | | | unknown [Zea mays] | | | | |
| | 1336-1353 | XP_002448678 | 242077483 | hypothetical protein SORBIDRAFT_06g031350 | 1 | Sorghum | 2259 | 2419 |
| | | | | [Sorghum bicolor] &gt:gi\|241939861\|gb\|EES13006.1\| | | bicolor | | |
| | | | | hypothetical protein SORBIDRAFT_06g031350 | | | | |
| | | | | [Sorghum bicolor] | | | | |
| | | ACV84254 | 258618872 | LOX6 [Sorghum bicolor] | 0.986711 | Sorghum | 2260 | 2420 |
| | | | | | | bicolor | | |
| | | NP_001105976 | 162463393 | lipoxygenase6 [Zea mays] | 0.864895 | Zea mays | 2261 | 2421 |
| | | | | &gt:gi\|84626289\|gb\|ABC59689.1\| lipoxygenase [Zea | | | | |
| | | | | mays] &gt:gi\|223949113\|gb\|ACN28640.1\| unknown | | | | |
| | | | | [Zea mays] &gt:gi\|224030589\|gb\|ACN34370.1\| | | | | |
| | | | | unknown [Zea mays] | | | | |
| | | | | &gt:gi\|238009650\|gb\|ACR35860.1\| unknown [Zea mays] | | | | |
| | | ACL5319 | 219885648 | unknown [Zea mays] | 0.844961 | Zea mays | 2262 | 2422 |
| | | | | &gt:gi\|224030301\|gb\|ACN34226.1\| unknown [Zea mays] | | | | |
| | | | | &gt:gi\|224030481\|gb\|ACN34316.1\| unknown | | | | |
| | | | | [Zea mays] | | | | |
| | 165-182 | NP_001145144 | 226499907 | hypothetical protein LOC100278375 [Zea mays] | 1 | Zea mays | 2263 | 2423 |
| | | | | &gt:gi\|195651893\|gb\|ACG45414.1\| hypothetical | | | | |
| | | | | protein [Zea mays] | | | | |
| | | XP_002444131 | 242078724 | hypothetical protein SORBIDRAFT_07g009350 | 0.875 | Sorghum | 2264 | 2424 |
| | | | | [Sorghum bicolor] &gt:gi\|241940481\|gb\|EES13626.1\| | | bicolor | | |
| | | | | hypothetical protein SORBIDRAFT_07g009350 | | | | |
| | | | | [Sorghum bicolor] | | | | |
| | 560-577 | NP_001169179 | 293336717 | hypothetical protein LOC100383029 [Zea mays] | 1 | Zea mays | 2265 | 2425 |
| | | | | &gt:gi\|223975337\|gb\|ACN31856.1\| unknown [Zea mays] | | | | |
| | | XP_002463802 | 242032814 | hypothetical protein SORBIDRAFT_01g006430 | 0.816964 | Sorghum | 2266 | 2426 |
| | | | | [Sorghum bicolor] &gt:gi\|241917656\|gb\|EER90800.1\| | | bicolor | | |
| | | | | hypothetical protein SORBIDRAFT_01g006430 | | | | |
| | | | | [Sorghum bicolor] | | | | |
| | 1379-1396 | XP_002458253 | 242058214 | hypothetical protein SORBIDRAFT_03g029970 | 1 | Sorghum | 2267 | 2427 |
| | | | | [Sorghum bicolor] &gt:gi\|241930228\|gb\|EES03373.1\| | | bicolor | | |
| | | | | hypothetical protein SORBIDRAFT_03g029970 | | | | |
| | | | | [Sorghum bicolor] | | | | |
| | | NP_001105776 | 162457954 | inorganic phosphate transporter 6 [Zea mays] | 0.927798 | Zea mays | 2268 | 2428 |
| | | | | &gt:gi\|60677369\|emb\|CAH25731.1\| phosphate | | | | |
| | | | | transporter [Zea mays] | | | | |
| | | | | &gt:gi\|65335927\|gb\|AAY42390.1\| inorganic phosphate | | | | |
| | | | | transporter 6 [Zea mays] | | | | |
| | | | | &gt:gi\|221047212\|gb\|ACL98053.1\| inorganic | | | | |
| | | | | phosphate transporter 6 [Zea mays] | | | | |
| | | NP_001043759 | 115438958 | Os01g0657100 [Oryza sativa Japonica Group] | 0.799639 | Oryza | 2269 | 2429 |
| | | | | &gt:gi\|75306389\|sp\|Q94DB8.1\|PT111_ORYSJ | | sativa | | |
| | | | | RecName: Full = Inorganic phosphate transporter 1-11; | | Japonica | | |
| | | | | Short = OsPT11; Short = OsPht1; 11; AltName: | | Group | | |
| | | | | Full = H(+)/Pi cotransporter | | | | |
| | | | | &gt:gi\|5290037\|dbj\|BAB63731.1\| putative phosphate | | | | |
| | | | | transport protein PT1 [Oryza sativa Japonica Group] | | | | |
| | | | | &gt:gi\|23600435\|gb\|AAN39041.1\| high affinity | | | | |
| | | | | phosphate transporter 11 [Oryza sativa Japonica Group] | | | | |
| | | | | &gt:gi\|23600477\|gb\|AAN39052.1\| putative | | | | |
| | | | | phosphate transporter OsPT11 [Oryza sativa Japonica | | | | |
| | | | | Group] &gt:gi\|113533290\|dbj\|BAF05673.1\| | | | | |
| | | | | Os01g0657100 [Oryza sativa Japonica Group] | | | | |
| | | | | &gt:gi\|125527126\|gb\|EAY75240.1\| hypothetical | | | | |
| | | | | protein OsI_03128 [Oryza sativa Indica Group] | | | | |
| | | | | &gt:gi\|125571445\|gb\|EAZ12960.1\| hypothetical | | | | |
| | | | | protein OsJ_02880 [Oryza sativa Japonica Group] | | | | |
| | | AAM14592 | 20162452 | phosphate transporter [Oryza sativa Indica Group] | 0.799639 | Oryza | 2270 | 2430 |
| | | | | | | sativa | | |
| | | | | | | Indica | | |
| | | | | | | Group | | |
| | 38-55 | ACL54346 | 219887942 | unknown [Zea mays] | 1 | Zea mays | 2271 | 2431 |
| | | NP_001144869 | 226528390 | hypothetical protein LOC100277964 [Zea mays] | 1 | Zea mays | 2272 | 2432 |
| | | | | &gt:gi\|195648236\|gb\|ACG43586.1\| hypothetical | | | | |
| | | | | protein [Zea mays] | | | | |
| | | XP_002464507 | 242034224 | hypothetical protein SORBIDRAFT_01g019820 | 0.820738 | Sorghum | 2273 | 2433 |
| | | | | [Sorghum bicolor] &gt:gi\|241918361\|gb\|EER91505.1\| | | bicolor | | |
| | | | | hypothetical protein SORBIDRAFT_01g019820 | | | | |
| | | | | [Sorghum bicolor] | | | | |
| | 241-258 | XP_002446139 | 242072405 | hypothetical protein SORBIDRAFT_06g002240 | 1 | Sorghum | 2274 | 2434 |
| | | | | [Sorghum bicolor] &gt:gi\|241937322\|gb\|EES10467.1\| | | bicolor | | |
| | | | | hypothetical protein SORBIDRAFT_06g002240 | | | | |
| | | | | [Sorghum bicolor] | | | | |
| | | ACN34366 | 224030580 | unknown [Zea mays] | 0.936508 | Zea mays | 2275 | 2435 |
| | | NP_001147785 | 226500959 | amino acid permease [Zea mays] | 0.924162 | Zea mays | 2276 | 2436 |
| | | | | &gt:gi\|195613758\|gb\|ACG28709.1\| amino acid | | | | |
| | | | | permease [Zea mays] | | | | |
| | | XP_002453115 | 242063651 | hypothetical protein SORBIDRAFT_04g000290 | 0.742504 | Sorghum | 2277 | 2437 |
| | | | | [Sorghum bicolor] &gt:gi\|241932946\|gb\|EES06091.1\| | | bicolor | | |
| | | | | hypothetical protein SORBIDRAFT_04g000290 | | | | |
| | | | | [Sorghum bicolor] | | | | |
| | | XP_002438439 | 242095897 | hypothetical protein SORBIDRAFT_10g019640 | 0.724868 | Sorghum | 2278 | 2438 |
| | | | | [Sorghum bicolor] &gt:gi\|241916662\|gb\|EER89806.1\| | | bicolor | | |
| | | | | hypothetical protein SORBIDRAFT_10g019640 | | | | |
| | | | | [Sorghum bicolor] | | | | |
| | | NP_001045585 | 115443610 | Os02g0101000 [Oryza sativa Japonica Group] | 0.705467 | Oryza | 2279 | 2439 |
| | | | | &gt:gi\|41053220\|dbj\|BAD08181.1\| putative amino acid | | sativa | | |
| | | | | transport protein [Oryza sativa Japonica Group] | | Japonica | | |
| | | | | | | Group | | |
| | | | | &gt:gi\|113535116\|dbj\|BAF07499.1\| Os02g0101000 | | | | |
| | | | | [Oryza sativa Japonica Group] | | | | |
| | | | | &gt:gi\|215704334\|dbj\|BAG93768.1\| unnamed protein | | | | |
| | | | | product [Oryza sativa Japonica Group] | | | | |
| | | | | &gt:gi\|218189857\|gb\|EEC72284.1\| hypothetical | | | | |
| | | | | protein OsI_05452 [Oryza sativa Indica Group] | | | | |
| | | | | &gt:gi\|222621988\|gb\|EEE56120.1\| hypothetical protein | | | | |
| | | | | OsJ_04987 [Oryza sativa Japonica Group] | | | | |
| | | NP_001136459 | 219362430 | hypothetical protein LOC100216569 [Zea mays] | 0.717813 | Zea mays | 2280 | 2440 |
| | | | | &gt:gi\|94695786\|gb\|ACF81977.1\| unknown | | | | |
| | | | | [Zea mays] | | | | |
| | 110-127 | ACG28457 | 195613253 | chlorophyll a-b binding protein 8 [Zea mays] | 1 | Zea mays | 2281 | 2441 |
| | | | | &gt:gi\|238013956\|gb\|ACR38013.1\| unknown | | | | |
| | | | | [Zea mays] | | | | |
| | | ABJ99590 | 116519120 | type III chlorophyll a/b-binding protein [Lycoris aurea] | 0.861423 | Lycoris | 2282 | 2442 |
| | | | | | | aurea | | |
| | | ABJ99591 | 116519124 | type III chlorophyll a/b-binding protein [Lycoris aurea] | 0.853933 | Lycoris | 2283 | 2443 |
| | | | | | | aurea | | |
| | | 1609235A | | chlorophyll a/b binding protein | 0.835206 | Solanum | 2284 | |
| | | | | | | lycopersicum | | |
| | | P27522 | | RecName: Full = Chlorophyll a-b binding protein 8, | 0.835206 | Solanum | 2285 | |
| | | | | chloroplastic; AltName: Full = LHCI type III CAB-8; | | lycopersicum | | |
| | | | | Flags: Precursor &gt:gi\|19182\|emb\|CAA33330.1\| Type | | | | |
| | | | | III chlorophyll a/b-binding protein [Solanum lycopersicum] | | | | |
| | | XP_002273201 | 225436256 | PREDICTED: hypothetical protein [Vitis vinifera] | 0.816479 | Vitis | 2286 | 2444 |
| | | | | | | vinifera | | |
| | | BAD25284 | 49388136 | putative chlorophyll a/b-binding protein type III | 0.827715 | Oryza | 2287 | 2445 |
| | | | | precursor [Oryza sativa Japonica Group] | | sativa | | |
| | | | | &gt:gi\|49388341\|dbj\|BAD25451.1\| putative | | Japonica | | |
| | | | | chlorophyll a/b-binding protein type III precursor | | Group | | |
| | | | | [Oryza sativa Japonica Group] | | | | |
| | | | | &gt:gi\|125538483\|gb\|EAY84878.1\| hypothetical | | | | |
| | | | | protein OsI_06243 [Oryza sativa Indica Group] | | | | |
| | | | | &gt:gi\|125581168\|gb\|EAZ22099.1\| hypothetical | | | | |
| | | | | protein OsJ_05758 [Oryza sativa Japonica Group] | | | | |
| | | | | &gt:gi\|215678980\|dbj\|BAG96410.1\| unnamed protein | | | | |
| | | | | product [Oryza sativa Japonica Group] | | | | |
| | | | | &gt:gi\|215679371\|dbj\|BAG96511.1\| unnamed protein | | | | |
| | | | | product [Oryza sativa Japonica Group] | | | | |
| | | | | &gt:gi\|215686386\|dbj\|BAG87647.1\| unnamed protein | | | | |
| | | | | product [Oryza sativa Japonica Group] | | | | |
| | | | | &gt:gi\|215737482\|dbj\|BAG96612.1\| unnamed protein | | | | |
| | | | | product [Oryza sativa Japonica Group] | | | | |
| | | | | &gt:gi\|215737505\|dbj\|BAG96635.1\| unnamed protein | | | | |
| | | | | product [Oryza sativa Japonica Group] | | | | |
| | | | | &gt:gi\|215737512\|dbj\|BAG96642.1\| unnamed protein | | | | |
| | | | | product [Oryza sativa Japonica Group] | | | | |
| | | | | &gt:gi\|215737560\|dbj\|BAG96690.1\| unnamed protein | | | | |
| | | | | product [Oryza sativa Japonica Group] | | | | |
| | | | | &gt:gi\|215737570\|dbj\|BAG96700.1\| unnamed protein | | | | |
| | | | | product [Oryza sativa Japonica Group] | | | | |
| | | | | &gt:gi\|215737580\|dbj\|BAG96710.1\| unnamed protein | | | | |
| | | | | product [Oryza sativa Japonica Group] | | | | |
| | | | | &gt:gi\|215737621\|dbj\|BAG96751.1\| unnamed protein | | | | |
| | | | | product [Oryza sativa Japonica Group] | | | | |
| | | | | &gt:gi\|215737634\|dbj\|BAG96764.1\| unnamed protein | | | | |
| | | | | product [Oryza sativa Japonica Group] | | | | |
| | | | | &gt:gi\|215737653\|dbj\|BAG96783.1\| unnamed protein | | | | |
| | | | | product [Oryza sativa Japonica Group] | | | | |
| | | | | &gt:gi\|215737729\|dbj\|BAG96859.1\| unnamed protein | | | | |
| | | | | product [Oryza sativa Japonica Group] | | | | |
| | | | | &gt:gi\|215737785\|dbj\|BAG96915.1\| unnamed protein | | | | |
| | | | | product [Oryza sativa Japonica Group] | | | | |
| | | | | &gt:gi\|215765646\|dbj\|BAG87343.1\| unnamed protein | | | | |
| | | | | product [Oryza sativa Japonica Group] | | | | |
| | | | | &gt:gi\|215767462\|dbj\|BAG99690.1\| unnamed protein | | | | |
| | | | | product [Oryza sativa Japonica Group] | | | | |
| | | XP_002888084 | 297840404 | hypothetical protein ARALYDRAFT_475174 | 0.805243 | Arabidopsis | 2288 | 2446 |
| | | | | [Arabidopsis lyrata subsp. lyrata] | | lyrata | | |
| | | | | &gt:gi\|297333925\|gb\|EFH64343.1\| hypothetical | | subsp. | | |
| | | | | protein ARALYDRAFT_475174 [Arabidopsis lyrata | | lyrata | | |
| | | | | subsp. lyrata] | | | | |
| | | NP_176347 | 30696581 | light-harvesting complex I chlorophyll a/b binding | 0.801498 | Arabidopsis | 2289 | 2447 |
| | | | | protein 3 [Arabidopsis thaliana] | | thaliana | | |
| | | | | &gt:gi\|334183551\|ref\|NP_001185280.1\| light- | | | | |
| | | | | harvesting complex I chlorophyll a/b binding protein 3 | | | | |
| | | | | [Arabidopsis thaliana] | | | | |
| | | | | &gt:gi\|4585882\|gb\|AAD25555.1\|AC005850_12PSI | | | | |
| | | | | type III chlorophyll a/b-binding protein [Arabidopsis thaliana] | | | | |
| | | | | &gt:gi\|16649019\|gb\|AAL24361.1\| PSI type | | | | |
| | | | | III chlorophyll a/b-binding protein [Arabidopsis thaliana] | | | | |
| | | | | &gt:gi\|20260044\|gb\|AAM13369.1\| PSI type | | | | |
| | | | | III chlorophyll a/b-binding protein [Arabidopsis thaliana] | | | | |
| | | | | &gt:gi\|332195725\|gb\|AEE33846.1\| light- | | | | |
| | | | | harvesting complex I chlorophyll a/b binding protein 3 | | | | |
| | | | | [Arabidopsis thaliana] | | | | |
| | | | | &gt:gi\|332195727\|gb\|AEE33848.1\| light-harvesting | | | | |
| | | | | complex I chlorophyll a/b binding protein 3 | | | | |
| | | | | [Arabidopsis thaliana] | | | | |
| | | XP_002321218 | 224131949 | light-harvesting complex I protein Lhca3 [Populus | 0.805243 | Populus | 2290 | 2448 |
| | | | | trichocarpa] &gt:gi\|118485656\|gb\|ABK94678.1\| | | trichocarpa | | |
| | | | | unknown [Populus trichocarpa] | | | | |
| | | | | &gt:gi\|222861991\|gb\|EEE99533.1\| light-harvesting | | | | |
| | | | | complex I protein Lhca3 [Populus trichocarpa] | | | | |
| | 211-228 | NP_001145838 | 226500499 | hypothetical protein LOC100279347 [Zea mays] | 1 | Zea mays | 2291 | 2449 |
| | | | | &gt:gi\|219884649\|gb\|ACL52699.1\| unknown | | | | |
| | | | | [Zea mays] | | | | |
| | 437-454 | NP_001060945 | 115474696 | Os08g0135900 [Oryza sativa Japonica Group] | 1 | Oryza | 2292 | 2450 |
| | | | | &gt:gi\|38636821\|dbj\|BAD03061.1\| putative tryptophan | | sativa | | |
| | | | | synthase beta-subunit [Oryza sativa Japonica Group] | | Japonica | | |
| | | | | &gt:gi\|46390803\|dbj\|BAD16309.1\| putative tryptophan | | Group | | |
| | | | | synthase beta-subunit [Oryza sativa Japonica Group] | | | | |
| | | | | &gt:gi\|113622914\|dbj\|BAF22859.1\| Os08g0135900 | | | | |
| | | | | [Oryza sativa Japonica Group] | | | | |
| | | | | &gt:gi\|125602115\|gb\|EAZ41440.1\| hypothetical | | | | |
| | | | | protein OsJ_25962 [Oryza sativa Japonica Group] | | | | |
| | | | | &gt:gi\|215704181\|dbj\|BAG93021.1\| unnamed protein | | | | |
| | | | | product [Oryza sativa Japonica Group] | | | | |
| | | O50046 | | RecName: Full = Tryptophan synthase beta chain 2, | 0.753715 | Camptotheca | 2293 | |
| | | | | chloroplastic; Flags: Precursor | | acuminata | | |
| | | | | &gt:gi\|2792520\|gb\|AAB97087.1\| tryptophan synthase | | | | |
| | | | | beta subunit [Camptotheca acuminata ] | | | | |
| | | | | &gt:gi\|2801771\|gb\|AAB97526.1\| tryptophan synthase | | | | |
| | | | | beta [Camptotheca acuminata] | | | | |
| | | NP_194437 | 186514016 | tryptophan synthase beta chain [Arabidopsis thaliana] | 0.745223 | Arabidopsis | 2294 | 2451 |
| | | | | &gt:gi\|1174779\|sp\|P25269.2\|TRBP2_ARATH | | thaliana | | |
| | | | | RecName: Full = Tryptophan synthase beta chain 2, | | | | |
| | | | | chloroplastic; Flags: Precursor | | | | |
| | | | | &gt:gi\|166894\|gb\|AAA32879.1\| tryptophan synthase | | | | |
| | | | | beta-subunit [Arabidopsis thaliana] | | | | |
| | | | | &gt:gi\|4490703\|emb\|CAB38837.1\| tryptophan synthase | | | | |
| | | | | beta-subunit (TSB2) [Arabidopsis thaliana] | | | | |
| | | | | &gt:gi\|7269560\|emb\|CAB79562.1\| tryptophan synthase | | | | |
| | | | | beta-subunit (TSB2) [Arabidopsis thaliana] | | | | |
| | | | | &gt:gi\|21536585\|gb\|AAM60917.1\| tryptophan synthase | | | | |
| | | | | beta-subunit TSB2 [Arabidopsis thaliana] | | | | |
| | | | | &gt:gi\|27808592\|gb\|AAO24576.1\| At4g27070 | | | | |
| | | | | [Arabidopsis thaliana] | | | | |
| | | | | &gt:gi\|110736231\|dbj\|BAF00086.1\| tryptophan | | | | |
| | | | | synthase beta-subunit [Arabidopsis thaliana] | | | | |
| | | | | &gt:gi\|332659896\|gb\|AEE85296.1\| tryptophan | | | | |
| | | | | synthase beta chain [Arabidopsis thaliana] | | | | |
| | 671-688 | XP_002459719 | 242043695 | hypothetical protein SORBIDRAFT_02g009320 | 1 | Sorghum | 2295 | 2452 |
| | | | | [Sorghum bicolor] &gt:gi\|241923096\|gb\|EER96240.1\| | | bicolor | | |
| | | | | hypothetical protein SORBIDRAFT_02g009320 | | | | |
| | | | | [Sorghum bicolor] | | | | |
| | | NP_001148038 | 226505789 | PVR3-like protein [Zea mays] | 0.788462 | Zea mays | 2296 | 2453 |
| | | | | &gt:gi\|195615460\|gb\|ACG29560.1\| PVR3-like protein | | | | |
| | | | | [Zea mays] | | | | |
| | 237-254 | ACN28677 | 223949186 | unknown [Zea mays] | 1 | Zea mays | 2297 | 2454 |
| | | CAE00460 | 66346994 | branched-chain amino acid aminotransferase | 0.761787 | Hordeum | 2298 | 2455 |
| | | | | [Hordeum vulgare subsp. vulgare] | | vulgare | | |
| | | | | | | subsp. | | |
| | | | | | | vulgare | | |
| | | NP_001049461 | 297600593 | Os03g0231600 [Oryza sativa Japonica Group] | 0.771712 | Oryza | 2299 | 2456 |
| | | | | &gt:gi\|27311239\|gb\|AAO00685.1\| Unknown protein | | sativa | | |
| | | | | [Oryza sativa Japonica Group] | | Japonica | | |
| | | | | &gt:gi\|27356673\|gb\|AAO06962.1\| Putative branched- | | Group | | |
| | | | | chain amino acid aminotransferase [Oryza sativa | | | | |
| | | | | Japonica Group] &gt:gi\|108706990\|gb\|ABF94785.1\| | | | | |
| | | | | Branched-chain-amino-acid aminotransferase 5, | | | | |
| | | | | chloroplast precursor, putative, expressed [Oryza sativa | | | | |
| | | | | Japonica Group] &gt:gi\|113547932\|dbj\|BAF11375.1\| | | | | |
| | | | | Os03g0231600 [Oryza sativa Japonica Group] | | | | |
| | | | | &gt:gi\|125542999\|gb\|EAY89138.1\| hypothetical | | | | |
| | | | | protein OsI_10629 [Oryza sativa Indica Group] | | | | |
| | | | | &gt:gi\|215740973\|dbj\|BAG97468.1\| unnamed protein | | | | |
| | | | | product [Oryza sativa Japonica Group] | | | | |
| | | | | &gt:gi\|215767850\|db;\|BAH00079.1\| unnamed protein | | | | |
| | | | | product [Oryza sativa Japonica Group] | | | | |
| | | ABF94786 | 108705663 | Branched-chain-amino-acid aminotransferase 5, | 0.771712 | Oryza | 2300 | |
| | | | | chloroplast precursor, putative, expressed [Oryza sativa | | sativa | | |
| | | | | Japonica Group] | | Japonica | | |
| | | | | | | Group | | |
| | | EEE58645 | 54398660 | hypothetical protein OsJ_10024 [Oryza sativa Japonica | 0.74938 | Oryza | 2301 | |
| | | | | Group] | | sativa | | |
| | | | | | | Japonica | | |
| | | | | | | Group | | |
| | 237-254 | NP_001147646 | 226510491 | branched-chain-amino-acid aminotransferase [Zea | 1 | Zea mays | 2302 | 2457 |
| | | | | mays] &gt:gi\|195612830\|gb\|ACG28245.1\| branched- | | | | |
| | | | | chain-amino-acid aminotransferase [Zea mays] | | | | |
| | | ABF94786 | 108705663 | Branched-chain-amino-acid aminotransferase 5, | 0.754854 | Oryza | 2303 | |
| | | | | chloroplast precursor, putative, expressed [Oryza sativa | | sativa | | |
| | | | | Japonica Group] | | Japonica | | |
| | | | | | | Group | | |
| | 130-147 | XP_002467798 | 242040806 | hypothetical protein SORBIDRAFT_01g034300 | 1 | Sorghum | 2304 | 2458 |
| | | | | [Sorghum bicolor] &gt:gi\|241921652\|gb\|EER94796.1\| | | bicolor | | |
| | | | | hypothetical protein SORBIDRAFT_01g034300 | | | | |
| | | | | [Sorghum bicolor] | | | | |
| | | ACG35330 | 195626999 | cytochrome P450 CYP709H1 [Zea mays] | 0.897004 | Zea mays | 2305 | 2459 |
| | | BAJ94885 | 326498044 | predicted protein [Hordeum vulgare subsp. vulgare] | 0.807116 | Hordeum | 2306 | 2460 |
| | | | | | | vulgare | | |
| | | | | | | subsp. | | |
| | | | | | | vulgare | | |
| | 26-Sep | NP_001150615 | 226531913 | photoperiod responsive protein [Zea mays] | 1 | Zea mays | 2307 | 2461 |
| | | | | &gt:gi\|195640592\|gb\|ACG39764.1\| photoperiod | | | | |
| | | | | responsive protein [Zea mays] | | | | |
| | | BAK02161 | 326497152 | predicted protein [Hordeum vulgare subsp. vulgare] | 0.79955 | Hordeum | 2308 | 2462 |
| | | | | | | vulgare | | |
| | | | | | | subsp. | | |
| | | | | | | vulgare | | |
| | | XP_002446455 | 242073037 | hypothetical protein SORBIDRAFT_06g016300 | 0.837838 | Sorghum | 2309 | 2463 |
| | | | | [Sorghum bicolor] &gt:gi\|241937638\|gb\|EES10783.1\| | | bicolor | | |
| | | | | hypothetical protein SORBIDRAFT_06g016300 | | | | |
| | | | | [Sorghum bicolor] | | | | |
| | | NP_001052772 | 115458343 | Os04g0418500 [Oryza sativa Japonica Group] | 0.759009 | Oryza | 2310 | 2464 |
| | | | | &gt:gi\|38605834\|emb\|CAE02914.3\| | | sativa | | |
| | | | | OSJNBb0108J11.6 [Oryza sativa Japonica Group] | | Japonica | | |
| | | | | &gt:gi\|113564343\|dbj\|BAF14686.1\| Os04g0418500 | | Group | | |
| | | | | [Oryza sativa Japonica Group] | | | | |
| | | NP_001047062 | 115446564 | Os02g0540700 [Oryza sativa Japonica Group] | 0.70045 | Oryza | 2311 | 2465 |
| | | | | &gt:gi\|50251283\|dbj\|BAD28063.1\| putative | | sativa | | |
| | | | | photoperiod responsive protein [Oryza sativa Japonica | | Japonica | | |
| | | | | Group] &gt:gi\|113536593\|dbj\|BAF08976.1\| | | Group | | |
| | | | | Os02g0540700 [Oryza sativa Japonica Group] | | | | |
| | | EAY86195 | 54362548 | hypothetical protein OsI_07571 [Oryza sativa Indica | 0.70045 | Oryza | 2312 | |
| | | | | Group] | | sativa | | |
| | | | | | | Indica | | |
| | | | | | | Group | | |
| Predicted | 553-570 | NP_001105321 | 162460315 | homologue of Arabidopsis gene AGAMOUS [Zea mays] | 1 | Zea mays | 2313 | 2466 |
| siRNA | | | | &gt:gi\|309574\|gb\|AAA02933.1\| homologue of | | | | |
| 59453 | | | | Arabidopsis gene AGAMOUS [Zea mays] | | | | |
| | 1821-1838 | NP_001130613 | 212274880 | hypothetical protein LOC100191712 [Zea mays] | 1 | Zea mays | 2314 | 2467 |
| | | | | &gt:gi\|194689638\|gb\|ACF78903.1\| unknown | | | | |
| | | | | [Zea mays] | | | | |
| | | XP_002437583 | 242094185 | hypothetical protein SORBIDRAFT_10g029780 | 0.935018 | Sorghum | 2315 | 2468 |
| | | | | [Sorghum bicolor] &gt:gi\|241915806\|gb\|EER88950.1\| | | bicolor | | |
| | | | | hypothetical protein SORBIDRAFT_10g029780 | | | | |
| | | | | [Sorghum bicolor] | | | | |
| | | BAJ87739 | 326511450 | predicted protein [Hordeum vulgare subsp. vulgare] | 0.797834 | Hordeum | 2316 | 2469 |
| | | | | | | vulgare | | |
| | | | | | | subsp. | | |
| | | | | | | vulgare | | |
| | | NP_001058556 | 115469913 | Os06g0712300 [Oryza sativa Japonica Group] | 0.790614 | Oryza | 2317 | 2470 |
| | | | | &gt:gi\|53792883\|dbj\|BAD54060.1\| putative JD1 | | sativa | | |
| | | | | [Oryza sariva Japonica Group] | | Japonica | | |
| | | | | &gt:gi\|113596596\|dbj\|BAF20470.1\| Os06g0712300 | | Group | | |
| | | | | [Oryza sativa Japonica Group] | | | | |
| | | EEC81300 | 54362548 | hypothetical protein OsI_24436 [Oryza sativa Indica | 0.787004 | Oryza | 2318 | |
| | | | | Group] | | sativa | | |
| | | | | | | Indica | | |
| | | | | | | Group | | |
| | | EEE66342 | 54398660 | hypothetical protein OsJ_22632 [Oryza sativa Japonica | 0.725632 | Oryza | 2319 | |
| | | | | Group] | | sativa | | |
| | | | | | | Japonica | | |
| | | | | | | Group | | |
| | 1068-1085 | NP_001159145 | 259490077 | hypothetical protein LOC100304227 [Zea mays] | 1 | Zea mays | 2320 | 2471 |
| | | | | &gt:gi\|223942267\|gb\|ACN25217.1\| unknown | | | | |
| | | | | [Zea mays] | | | | |
| | 65-82 | ACN25589 | 223943010 | unknown [Zea mays] | 1 | Zea mays | 2321 | 2472 |
| | | NP_001149752 | 226531311 | receptor-like protein kinase RK20-1 [Zea mays] | 1 | Zea mays | 2322 | 2473 |
| | | | | &gt:gi\|195631560\|gb\|ACG36675.1\| receplor-like | | | | |
| | | | | protein kinase RK20-1 [Zea mays] | | | | |
| | | NP_001140883 | 226532087 | hypothetical protein LOC100272959 [Zea mays] | 1 | Zea mays | 2323 | 2474 |
| | | | | &gt:gi\|194701580\|gb\|ACF84874.1\| unknown | | | | |
| | | | | [Zea mays] | | | | |
| | | XP_002462965 | 242050441 | hypothetical protein SORBIDRAFT_02g035450 | 0.738617 | Sorghum | 2324 | 2475 |
| | | | | [Sorghum bicolor] &gt:gi\|241926342\|gb\|EER99486.1\| | | bicolor | | |
| | | | | hypothetical protein SORBIDRAFT_02g035450 | | | | |
| | | | | [Sorghum bicolor] | | | | |
| | | XP_002462964 | 242050439 | hypothetical protein SORBIDRAFT_02g035440 | 0.74199 | Sorghum | 2325 | 2476 |
| | | | | [Sorghum bicolor] &gt:gi\|241926341\|gb\|EER99485.1\| | | bicolor | | |
| | | | | hypothetical protein SORBIDRAFT_02g035440 | | | | |
| | | | | [Sorghum bicolor] | | | | |
| | | BAJ96500 | 326517015 | predicted protein [Hordeum vulgare subsp. vulgare] | 0.706577 | Hordeum | 2326 | 2477 |
| | | | | | | vulgare | | |
| | | | | | | subsp. | | |
| | | | | | | vulgare | | |
| Predicted | 787-808 | EEC67667 | 54362548 | hypothetical protein OsI_35091 [Oryza sativa Indica | 1 | Oryza | 2327 | |
| zma | | | | Group] | | sativa | | |
| mir | | | | | | Indica | | |
| 47990 | | | | | | Group | | |
| | | NP_001065737 | 115484150 | Os11g0146700 [Oryza sativa Japonica Group] | 0.98741 | Oryza | 2328 | 2478 |
| | | | | &gt:gi\|108863991\|gb\|ABA91466.2\| expressed protein | | sativa | | |
| | | | | [Oryza sativa Japonica Group] | | Japonica | | |
| | | | | &gt:gi\|113644441\|dbj\|BAF27582.1\| Os11g0146700 | | Group | | |
| | | | | [Oryza sativa Japonica Group] | | | | |
| | | | | &gt:gi\|215704119\|dbj\|BAG92959.1\| unnamed protein | | | | |
| | | | | product [Oryza sativa Japonica Group] | | | | |
| | | | | &gt:gi\|222615514\|gb\|EEE51646.1\| hypothetical protein | | | | |
| | | | | OsJ_32953 [Oryza sativa Japonica Group] | | | | |
| | | XP_002441707 | 242082563 | hypothetical protein SORBIDRAFT_08g001030 | 0.839928 | Sorghum | 2329 | 2479 |
| | | | | [Sorghum bicolor] &gt:gi\|241942400\|gb\|EES15545.1\| | | bicolor | | |
| | | | | hypothetical protein SORBIDRAFT_08g001030 | | | | |
| | | | | [Sorghum bicolor] | | | | |
| | | EAY82236 | 54362548 | hypothetical protein OsI_37441 [Oryza sativa Indica | 0.890288 | Oryza | 2330 | |
| | | | | Group] | | sativa | | |
| | | | | | | Indica | | |
| | | | | | | Group | | |
| | | NP_001105988 | 162458579 | putative splicing factor [Zea mays] | 0.841727 | Zea mays | 2331 | 2480 |
| | | | | &gt:gi\|134035227\|gb\|ABO47657.1\| putative splicing | | | | |
| | | | | factor [Zea mays] &gt:gi\|134035229\|gb\|ABO47658.1\| | | | | |
| | | | | putative splicing factor [Zea mays] | | | | |
| | | ACG42564 | 195646191 | hypothetical protein [Zea mays] | 0.838129 | Zea mays | 2332 | 2481 |
| | | NP_001066137 | 297612660 | Os12g0143400 [Oryza sativa Japonica Group] | 0.877698 | Oryza | 2333 | 2482 |
| | | | | &gt:gi\|77553658\|gb\|ABA96454.1\| expressed protein | | sativa | | |
| | | | | [Oryza sativa Japonica Group] | | Japonica | | |
| | | | | &gt:gi\|125578472\|gb\|EAZ19618.1\| hypothetical | | Group | | |
| | | | | protein OsJ_35195 [Oryza sativa Japonica Group] | | | | |
| | | | | &gt:gi\|255670045\|dbj\|BAF29156.2\| Os12g0143400 | | | | |
| | | | | [Oryza sativa Japonica Group] | | | | |
| | | BAJ86249 | 326503485 | predicted protein [Hordeum vulgare subsp. vulgare] | 0.827338 | Hordeum | 2334 | 2483 |
| | | | | | | vulgare | | |
| | | | | | | subsp. | | |
| | | | | | | vulgare | | |
| | | XP_002274576 | 225444598 | PREDICTED: hypothetical protein isoform 1 [Vitis | 0.733813 | Vitis | 2335 | 2484 |
| | | | | vinifera] &gt:gi\|297738505\|emb\|CBI27750.3\| unnamed | | vinifera | | |
| | | | | protein product [Vitis vinifera] | | | | |
| Predicted | 222-242 | XP_002456622 | 242054952 | hypothetical protein SORBIDRAFT_03g039570 | 1 | Sorghum | 2336 | 2485 |
| zma | | | | [Sorghum bicolor] &gt:gi\|241928597\|gb\|EES01742.1\| | | bicolor | | |
| mir | | | | hypothetical protein SORBIDRAFT_03g039570 | | | | |
| 48459 | | | | [Sorghum bicolor] | | | | |
| | | ACG47007 | 195655078 | L-ascorbate oxidase precursor [Zea mays] | 0.939551 | Zea mays | 2337 | 2486 |
| | | NP_001105921 | 162463583 | putative laccase [Zea mays] | 0.915371 | Zea mays | 2338 | 2487 |
| | | | | &gt:gi\|84618783\|emb\|CAJ30497.1\| putative laccase | | | | |
| | | | | [Zea mays] | | | | |
| | | NP_001105874 | 162461267 | putative laccase [Zea mays] | 0.721934 | Zea mays | 2339 | 2488 |
| | | | | &gt:gi\|84618777\|emb\|CAJ30498.1\| putative laccase | | | | |
| | | | | [Zea mays] | | | | |
| | | Q0DHL5 | | RecName: Full = Putative laccase-11; AltName: | 0.715026 | Oryza | 2340 | |
| | | | | Full = Benzenediol: oxygen oxidoreductase 11; | | sativa | | |
| | | | | AltName: Full = Diphenol oxidase 11; AltName: | | Japonica | | |
| | | | | Full = Urishiol oxidase 11 | | Group | | |
| | | | | &gt:gi\|222631843\|gb\|EEE63975.1\| hypothetical protein | | | | |
| | | | | OsJ_18801 [Oryza sativa Japonica Group] | | | | |
| | | XP_002441216 | 242090766 | hypothetical protein SORBIDRAFT_09g022460 | 0.71848 | Sorghum | 2341 | 2489 |
| | | | | [Sorghum bicolor] | | bicolor | | |
| | | | | &gt:gi\|241946501\|gb\|EES19646.1\| | | | | |
| | | | | hypothetical protein SORBIDRAFT_09g022460 | | | | |
| | | | | [Sorghum bicolor] | | | | |
| Predicted | 493-513 | NP_001151803 | 226532099 | tubulin--tyrosine ligase-like protein 12 [Zea mays] | 1 | Zea mays | 2342 | 2490 |
| zma | | | | &gt:gi\|195649775\|gb\|ACG44355.1\| tubulin--tyrosine | | | | |
| mir | | | | ligase-like protein 12 [Zea mays] | | | | |
| 48824 | | | | | | | | |
| | | NP_001049155 | 115451108 | Os03g0179000 [Oryza sativa Japonica Group] | 0.833716 | Oryza | 2343 | 2491 |
| | | | | &gt:gi\|108706494\|gb\|ABF94289.1\| Tubulin-tyrosine | | sativa | | |
| | | | | ligase family protein, expressed [Oryza sativa Japonica | | Japonica | | |
| | | | | Group] &gt:gi\|113547626\|dbj\|BAF11069.1\| | | Group | | |
| | | | | Os03g0179000 | | | | |
| | | | | [Oryza sativa Japonica Group] | | | | |
| | | EEE58434 | 54398660 | hypothetical protein OsJ_09642 [Oryza sativa Japonica | 0.833716 | Oryza | 2344 | |
| | | | | Group] | | sativa | | |
| | | | | | | Japonica | | |
| | | | | | | Group | | |
| | | ACN32043 | 223975710 | unknown [Zea mays] | 0.821101 | Zea mays | 2345 | 2492 |
| | | BAJ96002 | 326512041 | predicted protein [Hordeum vulgare subsp. vulgare] | 0.811927 | Hordeum | 2346 | 2493 |
| | | | | | | vulgare | | |
| | | | | | | subsp. | | |
| | | | | | | vulgare | | |
| | | EEC74627 | 54362548 | hypothetical protein OsI_10249 [Oryza sativa Indica | 0.794725 | Oryza | 2347 | |
| | | | | Group] | | sativa | | |
| | | | | | | Indica | | |
| | | | | | | Group | | |
| | | AAO13468 | 26665711 | Hypothetical protein [Oryza sativa Japonica Group] | 0.787844 | Oryza | 2348 | |
| | | | | | | sativa | | |
| | | | | | | Japonica | | |
| | | | | | | Group | | |
| | | ABF94288 | 108705663 | Tubulin-tyrosine ligase family protein, expressed | 0.744266 | Oryza | 2349 | |
| | | | | [Oryza sativa Japonica Group] | | sativa | | |
| | | | | | | Japonica | | |
| | | | | | | Group | | |
| Predicted | 639-659 | XP_002436323 | 242091665 | hypothetical protein SORBIDRAFT_10g000450 | 1 | Sorghum | 2350 | 2494 |
| zma | | | | [Sorghum bicolor] &gt:gi\|241914546\|gb\|EER87690.1\| | | bicolor | | |
| mir | | | | hypothetical protein SORBIDRAFT_10g000450 | | | | |
| 49862 | | | | [Sorghum bicolor] | | | | |
| | | NP_001056548 | 115465897 | Os06g0104100 [Oryza sativa Japonica Group] | 0.847682 | Oryza | 2351 | 2495 |
| | | | | &gt:gi\|24413977\|dbj\|BAC22228.1\| unknown protein | | sativa | | |
| | | | | [Oryza sativa Japonica Group] | | Japonica | | |
| | | | | &gt:gi\|113594588\|dbj\|BAF18462.1\| Os06g0104100 | | Group | | |
| | | | | [Oryza sativa Japonica Group] | | | | |
| | | | | &gt:gi\|215766729\|dbj\|BAG98957.1\| unnamed protein | | | | |
| | | | | product [Oryza sativa Japonica Group] | | | | |
| | | | | &gt:gi\|218197401\|gb\|EEC79828.1\| hypothetical | | | | |
| | | | | protein OsI_21284 [Oryza sativa Indica Group] | | | | |
| | | | | &gt:gi\|222634813\|gb\|EEE64945.1\| hypothetical protein | | | | |
| | | | | OsJ_19819 [Oryza sativa Japonica Group] | | | | |
| | | BAK06666 | 326504749 | predicted protein [Hordeum vulgare subsp. vulgare] | 0.847682 | Hordeum | 2352 | 2496 |
| | | | | | | vulgare | | |
| | | | | | | subsp. | | |
| | | | | | | vulgare | | |
| | | NP_001151045 | 226532945 | phytoene synthase [Zea mays] | 0.86755 | Zea mays | 2353 | 2497 |
| | | | | &gt:gi\|195643862\|gb\|ACG41399.1\| phytoene synthase | | | | |
| | | | | [Zea mays] | | | | |
| | | ACF84120 | 194700071 | unknown [Zea mays] | 0.768212 | Zea mays | 2354 | 2498 |
| | | | | &gt:gi\|224028481\|gb\|ACN33316.1\| unknown [Zea | | | | |
| | | | | mays] &gt:gi\|224033023\|gb\|ACN35587.1\| unknown | | | | |
| | | | | [Zea mays] | | | | |
| | | NP_001132196 | 212722785 | hypothetical protein LOC100193624 [Zea mays] | 0.768212 | Zea mays | 2355 | 2499 |
| | | | | &gt:gi\|194692646\|gb\|ACF80407.1\| unknown [Zea mays] | | | | |
| | | | | &gt:gi\|194693728\|gb\|ACF80948.1\| unknown | | | | |
| | | | | [Zea mays] &gt:gi\|224035777\|gb\|ACN36964.1\| | | | | |
| | | | | unknown [Zea mays] | | | | |
| | | | | &gt:gi\|238014538\|gb\|ACR38304.1\| unknown [Zea mays] | | | | |

TABLE 22
Targets of small RNAs listed in Table 5 above

| | Mir | Homolog | Nucleotide | | | | | |
|---|---|---|---|---|---|---|---|---|
| Mir | Binding | NCBI | NCBI | | | | Protein | Nucleotide |
| name | Position | Accession | GI number | Annotation | Identity | Organism | seq id no: | seq id no: |
| Predicted | | XP_848677 | 74013161 | PREDICTED: similar to Retrovirus-related Pol | 1 | Canis familiaris | 2500 | 3970 |
| folded 24- | | | | polyprotein from transposon 297 [Canis familiaris] | | | | |
| nts-long | | | | | | | | |
| seq 51391 | | | | | | | | |
| | | AAL66751 | 18254408 | putative gag-pol precursor [Zea mays] | 0.79257 | Zea mays | 2501 | |
| | | | | &gt:gi\|33113975\|gb\|AAP94597.1\| putative gag-pol | | | | |
| | | | | precursor [Zea mays] | | | | |
| | | AAL75982 | 18568234 | putative prpol [Zea mays] | 0.806811 | Zea mays | 2502 | |
| Predicted | 215-238 | XP_002465068 | 242035346 | hypothetical protein SORBIDRAFT_01g031560 | 1 | Sorghum bicolor | 2503 | 3971 |
| folded 24- | | | | [Sorghum bicolor] | | | | |
| nts-long | | | | &gt:gi\|241918922\|gb\|EER92066.1\| | | | | |
| seq 52606 | | | | hypothetical protein SORBIDRAFT_01g031560 | | | | |
| | | | | [Sorghum bicolor] | | | | |
| | | NP_001147458 | 226528231 | LOC100281067 [Zea mays] | 0.847599 | Zea mays | 2504 | 3972 |
| | | | | &gt:gi\|195611562\|gb\|ACG27611.1\| indole-3-acetate | | | | |
| | | | | beta-glucosyltransferase [Zea mays] | | | | |
| | | BAK04808 | 326529722 | predicted protein | 0.73904 | Hordeum vulgare subsp. vulgare | 2505 | 3973 |
| | | | | [Hordeum vulgare subsp. vulgare] | | | | |
| | 372-395 | NP_001169556 | 293331934 | hypothetical protein LOC100383435 [Zea mays] | 1 | Zea mays | 2506 | 3974 |
| | | | | &gt:gi\|224030083\|gb\|ACN34117.1\| | | | | |
| | | | | unknown [Zea mays] | | | | |
| | | ACN35262 | 224032372 | unknown [Zea mays] | 1 | Zea mays | 2507 | 3975 |
| | | XP_002438139 | 242095297 | hypothetical protein SORBIDRAFT_10g008670 | 0.85906 | Sorghum bicolor | 2508 | 3976 |
| | | | | [Sorghum bicolor] | | | | |
| | | | | &gt:gi\|241916362\|gb\|EER89506.1\| | | | | |
| | | | | hypothetical protein SORBIDRAFT_10g008670 | | | | |
| | | | | [Sorghum bicolor] | | | | |
| | 211-234 | XP_002468043 | 242041296 | hypothetical protein SORBIDRAFT_01g038540 | 1 | Sorghum bicolor | 2509 | 3977 |
| | | | | [Sorghum bicolor] | | | | |
| | | | | &gt:gi\|241921897\|gb\|EER95041.1\| | | | | |
| | | | | hypothetical protein SORBIDRAFT_01g038540 | | | | |
| | | | | [Sorghum bicolor] | | | | |
| | | CAC13988 | 10880264 | phosphoinositide-specific phospholipase C | 0.906621 | Digitaria sanguinalis | 2510 | 3978 |
| | | | | [Digitaria sanguinalis] | | | | |
| | | ACF87551 | 194706933 | unknown [Zea mays] | 0.879457 | Zea mays | 2511 | 3979 |
| | | EEE58849 | 54398660 | hypothetical protein OsJ_10440 | 0.842105 | Oryza sativa Japonica Group | 2512 | |
| | | | | [Oryza sativa Japonica Group] | | | | |
| | | EEC75018 | 54362548 | hypothetical protein OsI_11098 | 0.83871 | Oryza sativa Indica Group | 2513 | |
| | | | | [Oryza sativa Indica Group] | | | | |
| | | NP_001049792 | 297600758 | Os03g0289300 [Oryza sativa Japonica Group] | 0.842105 | Oryza sativa Japonica Group | 2514 | 3980 |
| | | | | &gt:gi\|255674423\|dbj\|BAF11706.2\| Os03g0289300 | | | | |
| | | | | [Oryza sativa Japonica Group] | | | | |
| | | NP_001130371 | 212275184 | hypothetical protein LOC100191466 [Zea mays] | 0.726655 | Zea mays | 2515 | 3981 |
| | | | | &gt:gi\|194688956\|gb\|ACF78562.1\| | | | | |
| | | | | unknown [Zea mays] | | | | |
| | | ABF95389 | 108705663 | phosphoinositide-specific phospholipase C, putative, | 0.711375 | Oryza sativa Japonica Group | 2516 | 3982 |
| | | | | expressed [Oryza sativa Japonica Group] | | | | |
| | | XP_002463446 | 242051403 | hypothetical protein SORBIDRAFT_02g044010 | 0.721562 | Sorghum bicolor | 2517 | 3983 |
| | | | | [Sorghum bicolor] | | | | |
| | | | | &gt:gi\|241926823\|gb\|EER99967.1\| | | | | |
| | | | | hypothetical protein SORBIDRAFT_02g044010 | | | | |
| | | | | [Sorghum bicolor] | | | | |
| | 48-71 | ACG39668 | 195640399 | hypothetical protein [Zea mays] | 1 | Zea mays | 2518 | 3984 |
| | | NP_001130595 | 212276329 | hypothetical protein LOC100191694 [Zea mays] | 0.987562 | Zea mays | 2519 | 3985 |
| | | | | &gt:gi\|194689586\|gb\|ACF78877.1\| unknown | | | | |
| | | | | [Zea mays] | | | | |
| | | XP_002460510 | 242045277 | hypothetical protein SORBIDRAFT_02g029650 | 0.930348 | Sorghum bicolor | 2520 | 3986 |
| | | | | [Sorghum bicolor] | | | | |
| | | | | &gt:gi\|241923887\|gb\|EER97031.1\| | | | | |
| | | | | hypothetical protein SORBIDRAFT_02g029650 | | | | |
| | | | | [Sorghum bicolor] | | | | |
| | | NP_001063644 | 115480100 | Os09g0512700 [Oryza sativa Japonica Group] | 0.776119 | Oryza sativa Japonica Group | 2521 | 3987 |
| | | | | &gt:gi\|113631877\|dbj\|BAF25558.1\| Os09g0512700 | | | | |
| | | | | [Oryza sativa Japonica Group] | | | | |
| | | | | &gt:gi\|215765318\|dbj\|BAG87015.1\| unnamed protein | | | | |
| | | | | product [Oryza sativa Japonica Group] | | | | |
| | | EAZ09731 | 54362548 | hypothetical protein OsI_32019 | 0.773632 | Oryza sativa Indica Group | 2522 | |
| | | | | [Oryza sativa Indica Group] | | | | |
| | | BAK01473 | 326532193 | predicted protein [Hordeum vulgare subsp. vulgare] | 0.701493 | Hordeum vulgare subsp. vulgare | 2523 | 3988 |
| Predicted | 968-991 | NP_001168323 | 293337020 | hypothetical protein LOC100382090 [Zea mays] | 1 | Zea mays | 2524 | 3989 |
| folded 24- | | | | &gt:gi\|223947455\|gb\|ACN27811.1\| | | | | |
| nts-long | | | | unknown [Zea mays] | | | | |
| seq 52682 | | | | | | | | |
| | | XP_002447163 | 242074453 | hypothetical protein SORBIDRAFT_06g029640 | 0.881423 | Sorghum bicolor | 2525 | 3990 |
| | | | | [Sorghum bicolor] | | | | |
| | | | | &gt:gi\|241938346\|gb\|EES11491.1\| | | | | |
| | | | | hypothetical protein SORBIDRAFT_06g029640 | | | | |
| | | | | [Sorghum bicolor] | | | | |
| | | BAK03641 | 326515455 | predicted protein [Hordeum vulgare subsp. vulgare] | 0.758893 | Hordeum vulgare subsp. vulgare | 2526 | 3991 |
| | | CAH68069 | 90399260 | H0105C05.5 [Oryza sativa Indica Group] | 0.768775 | Oryza sativa Indica Group | 2527 | 3992 |
| | | CAI44640 | 58532108 | OSJNBb0015D13.11 [Oryza sativa Japonica Group] | 0.768775 | Oryza sativa Japonica Group | 2528 | 3993 |
| | | | | &gt:gi\|125549861\|gb\|EAY95683.1\| hypothetical | | | | |
| | | | | protein OsI_17548 [Oryza sativa Indica Group] | | | | |
| | | EAZ32093 | 54398660 | hypothetical protein OsJ_16284 | 0.766798 | Oryza sativa Japonica Group | 2529 | |
| | | | | [Oryza sativa Japonica Group] | | | | |
| | 604-627 | NP_001146934 | 226505051 | ovate protein [Zea mays] | 1 | Zea mays | 2530 | 3994 |
| | | | | &gt:gi\|195605346\|gb\|ACG24503.1\| ovate protein | | | | |
| | | | | [Zea mays] | | | | |
| Predicted | 130-151 | XP_002441411 | 242091156 | hypothetical protein SORBIDRAFT_09g026210 | 1 | Sorghum bicolor | 2531 | 3995 |
| siRNA | | | | [Sorghum bicolor] | | | | |
| 54566 | | | | &gt:gi\|241946696\|gb\|EES19841.1\| | | | | |
| | | | | hypothetical protein SORBIDRAFT_09g026210 | | | | |
| | | | | [Sorghum bicolor] | | | | |
| | | NP_001130268 | 212276204 | hypothetical protein LOC100191362 [Zea mays] | 0.956863 | Zea mays | 2532 | 3996 |
| | | | | &gt:gi\|194688708\|gb\|ACF78438.1\| unknown | | | | |
| | | | | [Zea mays] | | | | |
| | | | | &gt:gi\|195639384\|gb\|ACG39160.1\| | | | | |
| | | | | seed maturation protein [Zea mays] | | | | |
| | | | | &gt:gi\|19886847\|gb\|ACL53798.1\| unknown | | | | |
| | | | | [Zea mays] | | | | |
| | | NP_001056102 | 115465004 | Os05g0526400 [Oryza sativa Japonica Group] | 0.854902 | Oryza sativa Japonica Group | 2533 | 3997 |
| | | | | &gt:gi\|52353496\|gb\|AAU44062.1\| unknown protein | | | | |
| | | | | [Oryza sativa Japonica Group] | | | | |
| | | | | &gt:gi\|113579653\|dbj\|BAF18016.1\| Os05g0526400 | | | | |
| | | | | [Oryza sativa Japonica Group] | | | | |
| | | | | &gt:gi\|215737410\|dbj\|BAG96540.1\| unnamed protein | | | | |
| | | | | product [Oryza sativa Japonica Group] | | | | |
| | | | | &gt:gi\|215768410\|dbj\|BAH00639.1\| unnamed protein | | | | |
| | | | | product [Oryza sativa Japonica Group] | | | | |
| | | | | &gt:gi\|222632297\|gb\|EEE64429.1\| hypothetical | | | | |
| | | | | protein OsJ_19273 [Oryza sativa Japonica Group] | | | | |
| | | EAY98759 | 54362548 | hypothetical protein OsI_20691 | 0.839216 | Oryza sativa Indica Group | 2534 | |
| | | | | [Oryza sativa Indica Group] | | | | |
| | | NP_001149653 | 226529556 | LOC100283279 [Zea mays] | 0.827451 | Zea mays | 2535 | 3998 |
| | | | | &gt:gi\|195629032\|gb\|ACG36248.1\| seed | | | | |
| | | | | maturation protein [Zea mays] | | | | |
| | | | | &gt:gi\|223975305\|gb\|ACN31840.1\| unknown | | | | |
| | | | | [Zea mays] | | | | |
| | | BAJ89699 | 326487429 | predicted protein | 0.792157 | Hordeum vulgare subsp. vulgare | 2536 | 3999 |
| | | | | [Hordeum vulgare subsp. vulgare] | | | | |
| | | NP_001130478 | 212274956 | hypothetical protein LOC100191576 [Zea mays] | 0.811765 | Zea mays | 2537 | 4000 |
| | | | | &gt:gi\|194689240\|gb\|ACF78704.1\| unknown | | | | |
| | | | | [Zea mays] | | | | |
| | | | | &gt:gi\|194700430\|gb\|ACF84299.1\| unknown | | | | |
| | | | | [Zea mays] | | | | |
| | | | | &gt:gi\|195657175\|gb\|ACG48055.1\| seed | | | | |
| | | | | maturation protein [Zea mays] | | | | |
| | | | | &gt:gi\|238007632\|gb\|ACR34851.1\| unknown | | | | |
| | | | | [Zea mays] | | | | |
| | | EAY75742 | 54362548 | hypothetical protein OsI_03655 | 0.807843 | Oryza sativa Indica Group | 2538 | |
| | | | | [Oryza sativa Indica Group] | | | | |
| | | NP_001044177 | 115439794 | Os01g0736900 [Oryza sativa Japonica Group] | 0.8 | Oryza sativa Japonica Group | 2539 | 4001 |
| | | | | &gt:gi\|20160502\|dbj\|BAB89453.1\| putative 24 kDa | | | | |
| | | | | seed maturation protein [Oryza sativa Japonica Group] | | | | |
| | | | | &gt:gi\|113533708\|dbj\|BAF06091.1\| Os01g0736900 | | | | |
| | | | | [Oryza sativa Japonica Group] | | | | |
| | | | | &gt:gi\|215694473\|dbj\|BAG89426.1\| unnamed protein | | | | |
| | | | | product [Oryza sativa Japonica Group] | | | | |
| | | | | &gt:gi\|215768164\|dbj\|BAH00393.1\| unnamed protein | | | | |
| | | | | product [Oryza sativa Japonica Group] | | | | |
| | 74-95 | ACG32549 | 195621437 | seed maturation protein [Zea mays] | 1 | Zea mays | 2540 | 4002 |
| | 151-172 | XP_002463496 | 242032202 | hypothetical protein SORBIDRAFT_01g000790 | 1 | Sorghum bicolor | 2541 | 4003 |
| | | | | [Sorghum bicolor] | | | | |
| | | | | &gt:gi\|241917350\|gb\|EER90494.1\| | | | | |
| | | | | hypothetical protein SORBIDRAFT_01g000790 | | | | |
| | | | | [Sorghum bicolor] | | | | |
| | | NP_001144754 | 226493369 | hypothetical protein LOC100277804 [Zea mays] | 0.882834 | Zea mays | 2542 | 4004 |
| | | | | &gt:gi\|195646528\|gb\|ACG42732.1\| hypothetical | | | | |
| | | | | protein [Zea mays] | | | | |
| | | NP_001051933 | 115456664 | Os03g0853900 [Oryza sativa Japonica Group] | 0.722071 | Oryza sativa Japonica Group | 2543 | 4005 |
| | | | | &gt:gi\|29126338\|gb\|AAO66530.1\| putative p21 | | | | |
| | | | | C-terminal-binding protein (alternative splicing | | | | |
| | | | | products) [Oryza sativa Japonica Group] | | | | |
| | | | | &gt:gi\|108712161\|gb\|ABF99956.1\| expressed protein | | | | |
| | | | | [Oryza sativa Japonica Group] | | | | |
| | | | | &gt:gi\|113550404\|dbj\|BAF13847.1\| Os03g0853900 | | | | |
| | | | | [Oryza sativa Japonica Group] | | | | |
| | | | | &gt:gi\|125546494\|gb\|EAY92633.1\| hypothetical | | | | |
| | | | | protein OsI_14377 [Oryza sativa Indica Group] | | | | |
| | | | | &gt:gi\|125588683\|gb\|EAZ29347.1\| hypothetical | | | | |
| | | | | protein OsJ_13413 [Oryza sativa Japonica Group] | | | | |
| | | | | &gt:gi\|215737372\|dbj\|BAG96301.1\| unnamed protein | | | | |
| | | | | product [Oryza sativa Japonica Group] | | | | |
| | | BAK02069 | 326492571 | predicted protein [Hordeum vulgare subsp. vulgare] | 0.722071 | Hordeum vulgare subsp. vulgare | 2544 | 4006 |
| | | | | &gt:gi\|326505672\|dbj\|BAJ95507.1\| predicted protein | | | | |
| | | | | [Hordeum vulgare subsp. vulgare] | | | | |
| | 842-863 | NP_001152619 | 226506333 | lactoylglutathione lyase [Zea mays] | 1 | Zea mays | 2545 | 4007 |
| | | | | &gt:gi\|195658267\|gb\|ACG48601.1\| | | | | |
| | | | | lactoylglutathione lyase [Zea mays] | | | | |
| | | NP_001149571 | 226500125 | lactoylglutathione lyase [Zea mays] | 0.77027 | Zea mays | 2546 | 4008 |
| | | | | &gt:gi\|194700264\|gb\|ACF84216.1\| unknown | | | | |
| | | | | [Zea mays] | | | | |
| | | | | &gt:gi\|195628124\|gb\|ACG35892.1\| | | | | |
| | | | | lactoylglutathione lyase [Zea mays] | | | | |
| | | XP_002439371 | 242087076 | hypothetical protein SORBIDRAFT_09g005270 | 0.752252 | Sorghum bicolor | 2547 | 4009 |
| | | | | [Sorghum bicolor] | | | | |
| | | | | &gt:gi\|241944656\|gb\|EES17801.1\| | | | | |
| | | | | hypothetical protein SORBIDRAFT_09g005270 | | | | |
| | | | | [Sorghum bicolor] | | | | |
| | 23-Feb | XP_002466636 | 242038482 | hypothetical protein SORBIDRAFT_01g011390 | 1 | Sorghum bicolor | 2548 | 4010 |
| | | | | [Sorghum bicolor] | | | | |
| | | | | &gt:gi\|241920490\|gb\|EER93634.1\| | | | | |
| | | | | hypothetical protein SORBIDRAFT_01g011390 | | | | |
| | | | | [Sorghum bicolor] | | | | |
| | | NP_001105326 | 162460990 | indole-3-acetate beta-glucosyltransferase [Zea mays] | 0.833333 | Zea mays | 2549 | 4011 |
| | | | | &gt:gi\|2501499\|sp\|Q41819.1\|IABG_MAIZE | | | | |
| | | | | RecName: Full = Indole-3-acetate beta- | | | | |
| | | | | glucosyltransferase; AltName: Full = (Uridine 5&apos:- | | | | |
| | | | | diphosphate-glucose: indol-3-ylacetyl)-beta-D-glucosyl | | | | |
| | | | | transferase; AltName: Full = IAA-Glu synthase | | | | |
| | | | | &gt:gi\|548195\|gb\|AAA59054.1\| IAA-glu synthetase | | | | |
| | | | | [Zea mays] | | | | |
| | 213-234 | NP_001130342 | 212276285 | hypothetical protein LOC100191437 [Zea mays] | 1 | Zea mays | 2550 | 4012 |
| | | | | &gt:gi\|194688890\|gb\|ACF78529.1\| unknown | | | | |
| | | | | [Zea mays] | | | | |
| | 256-277 | ABF01001 | 93211179 | lipoxygenase [Zea mays] | 1 | Zea mays | 2551 | 4013 |
| | | ABF01002 | 93211181 | lipoxygenase [Zea mays] | 0.887293 | Zea mays | 2552 | 4014 |
| | | NP_001105981 | 162464185 | lipoxygenase11 [Zea mays] | 0.886188 | Zea mays | 2553 | 4015 |
| | | | | &gt:gi\|84626299\|gb\|ABC59694.1\| | | | | |
| | | | | lipoxygenase [Zea mays] | | | | |
| | | ACL53739 | 219886728 | unknown [Zea mays] | 0.80663 | Zea mays | 2554 | 4016 |
| | | ACN33257 | 224028362 | unknown [Zea mays] | 0.798895 | Zea mays | 2555 | 4017 |
| | | BAK01953 | 326492338 | predicted protein | 0.733702 | Hordeum vulgare subsp. vulgare | 2556 | 4018 |
| | | | | [Hordeum vulgare subsp. vulgare] | | | | |
| | | Q8GSM2 | | RecName: Full = Lipoxygenase 2.3, chloroplastic; | 0.731492 | Hordeum vulgare subsp. vulgare | 2557 | |
| | | | | AltName: Full = LOX2: Hv: 3; Flags: Precursor | | | | |
| | | | | &gt:gi\|25809278\|emb\|CAD45187.1\| lipoxygenase 2 | | | | |
| | | | | [Hordeum vulgare subsp. vulgare] | | | | |
| | | NP_001046180 | 115444800 | Os02g0194700 [Oryza sativa Japonica Group] | 0.710497 | Oryza sativa Japonica Group | 2558 | 4019 |
| | | | | &gt:gi\|49388109\|dbj\|BAD25240.1\| putative | | | | |
| | | | | Lipoxygenase 2.3, chloroplast precursor | | | | |
| | | | | [Oryza sativa Japonica Group] | | | | |
| | | | | &gt:gi\|113535711\|dbj\|BAF08094.1\| | | | | |
| | | | | Os02g0194700 [Oryza sativa Japonica Group] | | | | |
| | 1784-1805 | XP_002461846 | 242048199 | hypothetical protein SORBIDRAFT_02g009170 | 1 | Sorghum bicolor | 2559 | 4020 |
| | | | | [Sorghum bicolor] | | | | |
| | | | | &gt:gi\|241925223\|gb\|EER98367.1\| | | | | |
| | | | | hypothetical protein SORBIDRAFT_02g009170 | | | | |
| | | | | [Sorghum bicolor] | | | | |
| | | NP_001183207 | 308081147 | hypothetical protein LOC100501591 [Zea mays] | 0.853949 | Zea mays | 2560 | 4021 |
| | | | | &gt:gi\|238010066\|gb\|ACR36068.1\| unknown | | | | |
| | | | | [Zea mays] | | | | |
| | | BAC84446 | 50508886 | putative receptor-type protein kinase LRK1 | 0.731744 | Oryza sativa Japonica Group | 2561 | |
| | | | | [Oryza sativa Japonica Group] | | | | |
| | | | | &gt:gi\|50509358\|dbj\|BAD30821.1\| | | | | |
| | | | | putative receptor-type protein kinase LRK1 | | | | |
| | | | | [Oryza sativa Japonica Group] | | | | |
| | | EAZ03505 | 54362548 | hypothetical protein OsI_25645 | 0.721311 | Oryza sativa Indica Group | 2562 | |
| | | | | [Oryza sativa Indica Group] | | | | |
| | | NP_001175142 | 297725556 | Os07g0283125 [Oryza sativa Japonica Group] | 0.722802 | Oryza sativa Japonica Group | 2563 | 4022 |
| | | | | &gt:gi\|125599841\|gb\|EAZ39417.1\| hypothetical | | | | |
| | | | | protein OsJ_23853 [Oryza sativa Japonica Group] | | | | |
| | | | | &gt:gi\|255677673\|dbj\|BAH93870.1\| Os07g0283125 | | | | |
| | | | | [Oryza sativa Japonica Group] | | | | |
| | 257-278 | NP_001105980 | 162464002 | lipoxygenase10 [Zea mays] | 1 | Zea mays | 2564 | 4023 |
| | | | | &gt:gi\|84626297\|gb\|ABC59693.1\| lipoxygenase | | | | |
| | | | | [Zea mays] | | | | |
| Predicted | 216-237 | NP_001131499 | 212720749 | hypothetical protein LOC100192836 [Zea mays] | 1 | Zea mays | 2565 | 4024 |
| siRNA | | | | &gt:gi\|194691702\|gb\|ACF79935.1\| unknown | | | | |
| 54666 | | | | [Zea mays] | | | | |
| | | XP_002465188 | 242035586 | hypothetical protein SORBIDRAFT_01g033660 | 0.710594 | Sorghum bicolor | 2566 | 4025 |
| | | | | [Sorghum bicolor] | | | | |
| | | | | &gt:gi\|241919042\|gb\|EER92186.1\| | | | | |
| | | | | hypothetical protein SORBIDRAFT_01g033660 | | | | |
| | | | | [Sorghum bicolor] | | | | |
| | 37-58 | XP_002465922 | 242037054 | hypothetical protein SORBIDRAFT_01g048270 | 1 | Sorghum bicolor | 2567 | 4026 |
| | | | | [Sorghum bicolor] | | | | |
| | | | | &gt:gi\|241919776\|gb\|EER92920.1\| | | | | |
| | | | | hypothetical protein SORBIDRAFT_01g048270 | | | | |
| | | | | [Sorghum bicolor] | | | | |
| | | NP_001149726 | 226502948 | 60S ribosomal protein L5-1 [Zea mays] | 0.900662 | Zea mays | 2568 | 4027 |
| | | | | &gt:gi\|195629822\|gb\|ACG36552.1\| 60S | | | | |
| | | | | ribosomal protein L5-1 [Zea mays] | | | | |
| | | ACR38045 | 238014019 | unknown [Zea mays] | 0.89404 | Zea mays | 2569 | 4028 |
| | | NP_001151284 | 226510053 | 60S ribosomal protein L5-1 [Zea mays] | 0.887417 | Zea mays | 2570 | 4029 |
| | | | | &gt:gi\|195645524\|gb\|ACG42230.1\| 60S | | | | |
| | | | | ribosomal protein L5-1 [Zea mays] | | | | |
| | | BAJ89012 | 326527920 | predicted protein | 0.887417 | Hordeum vulgare subsp. vulgare | 2571 | 4030 |
| | | | | [Hordeum vulgare subsp. vulgare] | | | | |
| | | A2WXX3 | | RecName: Full = 60S ribosomal protein L5-1 | 0.857616 | Oryza sativa Indica Group | 2572 | |
| | | | | &gt:gi\|218189527\|gb\|EEC71954.1\| hypothetical | | | | |
| | | | | protein OsI_04779 [Oryza sativa Indica Group] | | | | |
| | | NP_001045083 | 115441606 | Os01g0896800 [Oryza sativa Japonica Group] | 0.857616 | Oryza sativa Japonica Group | 2573 | 4031 |
| | | | | &gt:gi\|122222375\|sp\|Q0JGY1.1\|RL51_ORYSJ | | | | |
| | | | | RecName: Full = 60S ribosomal protein L5-1 | | | | |
| | | | | &gt:gi\|56785265\|dbj\|BAD82174.1\| putative ribosomal | | | | |
| | | | | protein L5 [Oryza sativa Japonica Group] | | | | |
| | | | | &gt:gi\|113534614\|dbj\|BAF06997.1\| Os01g0896800 | | | | |
| | | | | [Oryza sativa Japonica Group] | | | | |
| | | | | &gt:gi\|169244415\|gb\|ACA50481.1\| 60S ribosomal | | | | |
| | | | | protein L5 [Oryza sativa Japonica Group] | | | | |
| | | | | &gt:gi\|215737756\|dbj\|BAG96886.1\| unnamed protein | | | | |
| | | | | product [Oryza sativa Japonica Group] | | | | |
| | | BAD82173 | 22093582 | putative ribosomal protein L5 | 0.854305 | Oryza sativa Japonica Group | 2574 | |
| | | | | [Oryza sativa Japonica Group] | | | | |
| | | | | &gt:gi\|169244413\|gb\|ACA50480.1\| 60S | | | | |
| | | | | ribosomal protein L5 [Oryza sativa Japonica Group] | | | | |
| | | | | &gt:gi\|218189526\|gb\|EEC71953.1\| hypothetical | | | | |
| | | | | protein OsI_04778 [Oryza sativa Indica Group] | | | | |
| | | | | &gt:gi\|222619675\|gb\|EEE55807.1\| hypothetical | | | | |
| | | | | protein OsJ_04400 [Oryza sativa Japonica Group] | | | | |
| | | BAC06273 | 21952828 | putative 60S ribosomal protein L5 | 0.847682 | Oryza sativa Japonica Group | 2575 | |
| | | | | [Oryza sativa Japonica Group] | | | | |
| | | | | &gt:gi\|125572963\|gb\|EAZ14478.1\| | | | | |
| | | | | hypothetical protein OsJ_04401 | | | | |
| | | | | [Oryza sativa Japonica Group] | | | | |
| | | Q8L4L4 | | RecName: Full = 60S ribosomal protein L5-2 | 0.844371 | Oryza sativa Japonica Group | 2576 | |
| | | | | &gt:gi\|313103586\|pdb\|3IZ5\|Q Chain Q, Localization | | | | |
| | | | | Of The Large Subunit Ribosomal Proteins Into A 5.5 | | | | |
| | | | | A Cryo-Em Map Of Triticum Aestivum Translating | | | | |
| | | | | 80s Ribosome &gt:gi\|315113258\|pdb\|3IZR\|Q Chain | | | | |
| | | | | Q, Localization Of The Large Subunit Ribosomal | | | | |
| | | | | Proteins Into A 5.5 A Cryo-Em Map Of | | | | |
| | | | | Triticum Aestivum Translating 80s Ribosome | | | | |
| | | | | &gt:gi\|21952857\|dbj\|BAC06272.1\| putative 60S | | | | |
| | | | | ribosomal protein L5 [Oryza sativa Japonica Group] | | | | |
| | 292-313 | NP_001136748 | 219362396 | hypothetical protein LOC100216889 [Zea mays] | 1 | Zea mays | 2577 | 4032 |
| | | | | &gt:gi\|194696888\|gb\|ACF82528.1\| unknown | | | | |
| | | | | [Zea mays] | | | | |
| | | XP_002441226 | 242090786 | hypothetical protein SORBIDRAFT_09g022730 | 0.738806 | Sorghum bicolor | 2578 | 4033 |
| | | | | [Sorghum bicolor] | | | | |
| | | | | &gt:gi\|241946511\|gb\|EES19656.1\| | | | | |
| | | | | hypothetical protein SORBIDRAFT_09g022730 | | | | |
| | | | | [Sorghum bicolor] | | | | |
| | 376-397 | XP_002436516 | 242092051 | hypothetical protein SORBIDRAFT_10g004010 | 1 | Sorghum bicolor | 2579 | 4034 |
| | | | | [Sorghum bicolor] | | | | |
| | | | | &gt:gi\|241914739\|gb\|EER87883.1\| | | | | |
| | | | | hypothetical protein SORBIDRAFT_10g004010 | | | | |
| | | | | [Sorghum bicolor] | | | | |
| | | NP_001145738 | 226529448 | hypothetical protein LOC100279245 [Zea mays] | 0.920388 | Zea mays | 2580 | 4035 |
| | | | | &gt:gi\|219884233\|gb\|ACL52491.1\| unknown | | | | |
| | | | | [Zea mays] | | | | |
| | | NP_001159047 | 259490760 | hypothetical protein LOC100304073 [Zea mays] | 0.904854 | Zea mays | 2581 | 4036 |
| | | | | &gt:gi\|195644974\|gb\|ACG41955.1\| hypothetical | | | | |
| | | | | protein [Zea mays] | | | | |
| | | EEC80051 | 54362548 | hypothetical protein OsI_21754 | 0.786408 | Oryza sativa Indica Group | 2582 | |
| | | | | [Oryza sativa Indica Group] | | | | |
| | | EEE65126 | 54398660 | hypothetical protein OsJ_20196 | 0.782524 | Oryza sativa Japonica Group | 2583 | |
| | | | | [Oryza sativa Japonica Group] | | | | |
| | 362-383 | NP_001149452 | 226510424 | LOC100283078 [Zea mays] | 1 | Zea mays | 2584 | 4037 |
| | | | | &gt:gi\|195627344\|gb\|ACG35502.1\| | | | | |
| | | | | lipopolysaccharide-modifying protein | | | | |
| | | | | [Zea mays] | | | | |
| | | XP_002443683 | 242077828 | hypothetical protein SORBIDRAFT_07g000280 | 0.726667 | Sorghum bicolor | 2585 | 4038 |
| | | | | [Sorghum bicolor] | | | | |
| | | | | &gt:gi\|241940033\|gb\|EES13178.1\| | | | | |
| | | | | hypothetical protein SORBIDRAFT_07g000280 | | | | |
| | | | | [Sorghum bicolor] | | | | |
| | 211-232 | ACN33370 | 224028588 | unknown [Zea mays] | 1 | Zea mays | 2586 | 4039 |
| | | XP_002447369 | 242074865 | hypothetical protein SORBIDRAFT_06g033830 | 0.865526 | Sorghum bicolor | 2587 | 4040 |
| | | | | [Sorghum bicolor] | | | | |
| | | | | &gt:gi\|241938552\|gb\|EES11697.1\| | | | | |
| | | | | hypothetical protein SORBIDRAFT_06g033830 | | | | |
| | | | | [Sorghum bicolor] | | | | |
| | | CAB53491 | 5777612 | CAA303718.1 protein [Oryza sativa] | 0.735941 | Oryza sativa | 2588 | 4041 |
| | | | | &gt:gi\|90399023\|emb\|CAJ86143.1\| H0701F11.9 | | | | |
| | | | | [Oryza sativa Indica Group] | | | | |
| | | | | &gt:gi\|116311977\|emb\|CAJ86335.1\| H0814G11.2 | | | | |
| | | | | [Oryza sativa Indica Group] | | | | |
| | | EAY96137 | 54362548 | hypothetical protein OsI_18016 | 0.718826 | Oryza sativa Indica Group | 2589 | |
| | | | | [Oryza sativa Indica Group] | | | | |
| | | EEE61953 | 54398660 | hypothetical protein OsJ_16714 | 0.706601 | Oryza sativa Japonica Group | 2590 | |
| | | | | [Oryza sativa Japonica Group] | | | | |
| | 128-149 | XP_002451458 | 242060337 | hypothetical protein SORBIDRAFT_04g002290 | 1 | Sorghum bicolor | 2591 | 4042 |
| | | | | [Sorghum bicolor] | | | | |
| | | | | &gt:gi\|241931289\|gb\|EES04434.1\| | | | | |
| | | | | hypothetical protein SORBIDRAFT_04g002290 | | | | |
| | | | | [Sorghum bicolor] | | | | |
| | | NP_001146328 | 226493387 | hypothetical protein LOC100279904 [Zea mays] | 0.824798 | Zea mays | 2592 | 4043 |
| | | | | &gt:gi\|219886655\|gb\|ACL53702.1\| unknown | | | | |
| | | | | [Zea mays] | | | | |
| | 544-565 | XP_002454331 | 242066083 | hypothetical protein SORBIDRAFT_04g028790 | 1 | Sorghum bicolor | 2593 | 4044 |
| | | | | [Sorghum bicolor] | | | | |
| | | | | &gt:gi\|241934162\|gb\|EES07307.1\| | | | | |
| | | | | hypothetical protein SORBIDRAFT_04g028790 | | | | |
| | | | | [Sorghum bicolor] | | | | |
| | | NP_001057318 | 115467437 | Os06g0256900 [Oryza sativa Japonica Group] | 0.875 | Oryza sativa Japonica Group | 2594 | 4045 |
| | | | | &gt:gi\|75253245\|sp\|Q652F9.1\|GUN17_ORYSJ | | | | |
| | | | | RecName: Full = Endoglucanase 17; AltName: | | | | |
| | | | | Full = Endo-1,4-beta glucanase 17; AltName: | | | | |
| | | | | Full = OsGLU13; Flags: Precursor | | | | |
| | | | | &gt:gi\|52077266\|dbj\|BAD46308.1\| | | | | |
| | | | | putative endo-1,4-beta-glucanase precursor | | | | |
| | | | | [Oryza sativa Japonica Group] | | | | |
| | | | | &gt:gi\|113595358\|dbj\|BAF19232.1\| | | | | |
| | | | | Os06g0256900 [Oryza sativa Japonica Group] | | | | |
| | | | | &gt:gi\|125596749\|gb\|EAZ36529.1\| hypothetical | | | | |
| | | | | protein OsJ_20865 [Oryza sativa Japonica Group] | | | | |
| | | NP_001048033 | 115448506 | Os02g0733300 [Oryza sativa Japonica Group] | 0.890873 | Oryza sativa Japonica Group | 2595 | 4046 |
| | | | | &gt:gi\|75225300\|sp\|Q6Z2J3.1\|GUN6_ORYSJ | | | | |
| | | | | RecName: Full = Endoglucanase 6; AltName: | | | | |
| | | | | Full = Endo-1,4-beta glucanase 6; AltName: | | | | |
| | | | | Full = OsCel9E; Flags: Precursor | | | | |
| | | | | &gt:gi\|46390665\|dbj\|BAD16147.1\| | | | | |
| | | | | putative endo-1,4-beta-glucanase | | | | |
| | | | | [Oryza sativa Japonica Group] | | | | |
| | | | | &gt:gi\|113537564\|dbj\|BAF09947.1\| Os02g0733300 | | | | |
| | | | | [Oryza sativa Japonica Group] | | | | |
| | | | | &gt:gi\|215704889\|dbj\|BAG94917.1\| unnamed protein | | | | |
| | | | | product [Oryza sativa Japonica Group] | | | | |
| | | | | &gt:gi\|215740598\|dbj\|BAG97254.1\| unnamed protein | | | | |
| | | | | product [Oryza sativa Japonica Group] | | | | |
| | | ACG39876 | 195640815 | endoglucanase 1 precursor [Zea mays] | 0.934524 | Zea mays | 2596 | 4047 |
| | | BAJ85886 | 326495579 | predicted protein | 0.853175 | Hordeum vulgare subsp. vulgare | 2597 | 4048 |
| | | | | [Hordeum vulgare subsp. vulgare] | | | | |
| | | BAK01727 | 326489492 | predicted protein | 0.823413 | Hordeum vulgare subsp. vulgare | 2598 | 4049 |
| | | | | [Hordeum vulgare subsp. vulgare] | | | | |
| | | ACN25509 | 223942850 | unknown [Zea mays] | 0.801587 | Zea mays | 2599 | 4050 |
| | | NP_001136728 | 219363410 | hypothetical protein LOC100216867 [Zea mays] | 0.81746 | Zea mays | 2600 | 4051 |
| | | | | &gt:gi\|194699722\|gb\|ACF83945.1\| unknown | | | | |
| | | | | [Zea mays] | | | | |
| | 778-799 | XP_002437997 | 242095013 | hypothetical protein SORBIDRAFT_10g006130 | 1 | Sorghum bicolor | 2601 | 4052 |
| | | | | [Sorghum bicolor] | | | | |
| | | | | &gt:gi\|241916220\|gb\|EER89364.1\| | | | | |
| | | | | hypothetical protein SORBIDRAFT_10g006130 | | | | |
| | | | | [Sorghum bicolor] | | | | |
| | | AAK73111 | 166008041 | serine threonine kinase 1 [Zea mays] | 0.93593 | Zea mays | 2602 | |
| | | | | &gt:gi\|145309042\|gb\|ABP57732.1\| Tassel serine | | | | |
| | | | | threonine kinase 1 [Zea mays] | | | | |
| | | ACA21852 | 168251061 | serine threonine kinase 1 [Zea mays] | 0.896985 | Zea mays | 2603 | |
| | | EEE65245 | 54398660 | hypothetical protein OsJ_20418 | 0.815327 | Oryza sativa Japonica Group | 2604 | |
| | | | | [Oryza sativa Japonica Group] | | | | |
| | | BAK06108 | 326499234 | predicted protein | 0.802764 | Hordeum vulgare subsp. vulgare | 2605 | 4053 |
| | | | | [Hordeum vulgare subsp. vulgare] | | | | |
| | | EAY99981 | 54362548 | hypothetical protein OsI_21985 | 0.762563 | Oryza sativa Indica Group | 2606 | |
| | | | | [Oryza sativa Indica Group] | | | | |
| | 103-124 | XP_002468218 | 242041646 | hypothetical protein SORBIDRAFT_01g041990 | 1 | Sorghum bicolor | 2607 | 4054 |
| | | | | [Sorghum bicolor] | | | | |
| | | | | &gt:gi\|241922072\|gb\|EER95216.1\| | | | | |
| | | | | hypothetical protein SORBIDRAFT_01g041990 | | | | |
| | | | | [Sorghum bicolor] | | | | |
| | | NP_001150369 | 226490909 | LOC100283999 [Zea mays] | 0.962085 | Zea mays | 2608 | 4055 |
| | | | | &gt:gi\|195638716\|gb\|ACG38826.1\| | | | | |
| | | | | maf-like protein CV_0124 | | | | |
| | | | | [Zea mays] | | | | |
| | | BAJ91214 | 326505949 | predicted protein | 0.914692 | Hordeum vulgare subsp. vulgare | 2609 | 4056 |
| | | | | [Hordeum vulgare subsp. vulgare] | | | | |
| | | ABF94776 | 108705663 | Maf family protein, putative, expressed | 0.914692 | Oryza sativa Japonica Group | 2610 | 4057 |
| | | | | [Oryza sativa Japonica Group] | | | | |
| | | EEC74808 | 54362548 | hypothetical protein OsI_10622 | 0.881517 | Oryza sativa Indica Group | 2611 | |
| | | | | [Oryza sativa Indica Group] | | | | |
| | | | | &gt:gi\|222624509\|gb\|EEE58641.1\| | | | | |
| | | | | hypothetical protein OsJ_10016 | | | | |
| | | | | [Oryza sativa Japonica Group] | | | | |
| | 556-577 | NP_001170197 | 293334350 | hypothetical protein LOC100384147 [Zea mays] | 1 | Zea mays | 2612 | 4058 |
| | | | | &gt:gi\|224034241\|gb\|ACN36196.1\| unknown | | | | |
| | | | | [Zea mays] | | | | |
| | | XP_002468612 | 242042434 | hypothetical protein SORBIDRAFT_01g048980 | 0.928846 | Sorghum bicolor | 2613 | 4059 |
| | | | | [Sorghum bicolor] | | | | |
| | | | | &gt:gi\|241922466\|gb\|EER95610.1\| | | | | |
| | | | | hypothetical protein SORBIDRAFT_01g048980 | | | | |
| | | | | [Sorghum bicolor] | | | | |
| | | NP_001048804 | 115450406 | Os03g0123300 [Oryza sativa Japonica Group] | 0.857692 | Oryza sativa Japonica Group | 2614 | 4060 |
| | | | | &gt:gi\|108705924\|gb\|ABF93719.1\| Fizzy-related | | | | |
| | | | | protein, putative, expressed | | | | |
| | | | | [Oryza sativa Japonica Group] | | | | |
| | | | | &gt:gi\|113547275\|dbj\|BAF10718.1\| | | | | |
| | | | | Os03g0123300 [Oryza sativa Japonica Group] | | | | |
| | | | | &gt:gi\|215697879\|dbj\|BAG92072.1\| unnamed protein | | | | |
| | | | | product [Oryza sativa Japonica Group] | | | | |
| | | AAN74839 | 24942863 | Putative cell cycle switch protein | 0.851923 | Oryza sativa Japonica Group | 2615 | 4061 |
| | | | | [Oryza sativa Japonica Group] | | | | |
| | | BAK01604 | 326489240 | predicted protein | 0.778846 | Hordeum vulgare subsp. vulgare | 2616 | 4062 |
| | | | | [Hordeum vulgare subsp. vulgare] | | | | |
| | 1689-1710 | ACN35345 | 224032538 | unknown [Zea mays] | 1 | Zea mays | 2617 | 4063 |
| | 40-61 | ACR35596 | 238009121 | unknown [Zea mays] | 1 | Zea mays | 2618 | 4064 |
| | | NP_001167964 | 293337176 | hypothetical protein LOC100381680 [Zea mays] | 0.723005 | Zea mays | 2619 | 4065 |
| | | | | &gt:gi\|223945177\|gb\|ACN26672.1\| unknown | | | | |
| | | | | [Zea mays] | | | | |
| | 135-156 | XP_002437370 | 242093759 | hypothetical protein SORBIDRAFT_10g025700 | 1 | Sorghum bicolor | 2620 | 4066 |
| | | | | [Sorghum bicolor] | | | | |
| | | | | &gt:gi\|241915593\|gb\|EER88737.1\| | | | | |
| | | | | hypothetical protein SORBIDRAFT_10g025700 | | | | |
| | | | | [Sorghum bicolor] | | | | |
| | | NP_001169745 | 293337166 | hypothetical protein LOC100383626 [Zea mays] | 0.957878 | Zea mays | 2621 | 4067 |
| | | | | &gt:gi\|224031377\|gb\|ACN34764.1\| unknown | | | | |
| | | | | [Zea mays] | | | | |
| | | EEC81091 | 54362548 | hypothetical protein OsI_23922 | 0.840874 | Oryza sativa Indica Group | 2622 | |
| | | | | [Oryza sativa Indica Group] | | | | |
| | | NP_001058222 | 115469245 | Os06g0650600 [Oryza sativa Japonica Group] | 0.840874 | Oryza sativa Japonica Group | 2623 | 4068 |
| | | | | &gt:gi\|51534986\|dbj\|BAD38110.1\| endomembrane | | | | |
| | | | | protein 70-like [Oryza sativa Japonica Group] | | | | |
| | | | | &gt:gi\|113596262\|dbj\|BAF20136.1\| Os06g0650600 | | | | |
| | | | | [Oryza sativa Japonica Group] | | | | |
| | | | | &gt:gi\|215694831\|dbj\|BAG90022.1\| unnamed protein | | | | |
| | | | | product [Oryza sativa Japonica Group] | | | | |
| | | ACG29725 | 195615789 | transmembrane 9 superfamily protein member | 0.806552 | Zea mays | 2624 | 4069 |
| | | | | 4 [Zea mays] | | | | |
| | | NP_001146016 | 226492185 | hypothetical protein LOC100279547 [Zea mays] | 0.803432 | Zea mays | 2625 | 4070 |
| | | | | &gt:gi\|219885331\|gb\|ACL53040.1\| unknown | | | | |
| | | | | [Zea mays] | | | | |
| | | XP_002437369 | 242093757 | hypothetical protein SORBIDRAFT_10g025690 | 0.809672 | Sorghum bicolor | 2626 | 4071 |
| | | | | [Sorghum bicolor] | | | | |
| | | | | &gt:gi\|241915592\|gb\|EER88736.1\| | | | | |
| | | | | hypothetical protein SORBIDRAFT_10g025690 | | | | |
| | | | | [Sorghum bicolor] | | | | |
| | | XP_002445411 | 242081284 | hypothetical protein SORBIDRAFT_07g016310 | 0.798752 | Sorghum bicolor | 2627 | 4072 |
| | | | | [Sorghum bicolor] | | | | |
| | | | | &gt:gi\|241941761\|gb\|EES14906.1\| | | | | |
| | | | | hypothetical protein SORBIDRAFT_07g016310 | | | | |
| | | | | [Sorghum bicolor] | | | | |
| | | NP_001130383 | 212275585 | hypothetical protein LOC100191479 [Zea mays] | 0.794072 | Zea mays | 2628 | 4073 |
| | | | | &gt:gi\|194688986\|gb\|ACF78577.1\| unknown | | | | |
| | | | | [Zea mays] | | | | |
| | | | | &gt:gi\|195614790\|gb\|ACG29225.1\| | | | | |
| | | | | transmembrane 9 superfamily protein member 2 | | | | |
| | | | | precursor [Zea mays] | | | | |
| | | XP_002529382 | 255576994 | Endosomal P24A protein precursor, putative | 0.792512 | Ricinus communis | 2629 | 4074 |
| | | | | [Ricinus communis] | | | | |
| | | | | &gt:gi\|223531130\|gb\|EEF32978.1\| | | | | |
| | | | | Endosomal P24A protein precursor, putative | | | | |
| | | | | [Ricinus communis] | | | | |
| | 698-719 | ACR38139 | 238014207 | unknown [Zea mays] | 1 | Zea mays | 2630 | 4075 |
| | | ACG26030 | 195608399 | homeobox domain containing protein | 0.987302 | Zea mays | 2631 | 4076 |
| | | | | [Zea mays] | | | | |
| | | XP_002465335 | 242035880 | hypothetical protein SORBIDRAFT_01g036670 | 0.873016 | Sorghum bicolor | 2632 | 4077 |
| | | | | [Sorghum bicolor] | | | | |
| | | | | &gt:gi\|241919189\|gb\|EER92333.1\| | | | | |
| | | | | hypothetical protein SORBIDRAFT_01g036670 | | | | |
| | | | | [Sorghum bicolor] | | | | |
| | | NP_001049985 | 115452768 | Os03g0325600 [Oryza sativa Japonica Group] | 0.704762 | Oryza sativa Japonica Group | 2633 | 4078 |
| | | | | &gt:gi\|122247076\|sp\|Q10M29.1\|WOX6_ORYSJ | | | | |
| | | | | RecName: Full = WUSCHEL-related homeobox 6; | | | | |
| | | | | AltName: Full = OsWOX6 | | | | |
| | | | | &gt:gi\|108707914\|gb\|ABF95709.1\| Homeobox | | | | |
| | | | | domain containing protein, expressed | | | | |
| | | | | [Oryza sativa Japonica Group] | | | | |
| | | | | &gt:gi\|113548456\|dbj\|BAF11899.1\| | | | | |
| | | | | Os03g0325600 [Oryza sativa Japonica Group] | | | | |
| | | A2XG77 | | RecName: Full = WUSCHEL-related homeobox 6; | 0.704762 | Oryza sativa Indica Group | 2634 | |
| | | | | AltName: Full = OsWOX6 | | | | |
| | | | | &gt:gi\|125543698\|gb\|EAY89837.1\| | | | | |
| | | | | hypothetical protein OsI_11385 | | | | |
| | | | | [Oryza sativa Indica Group] | | | | |
| | 328-349 | NP_001130841 | 212276171 | hypothetical protein LOC100191945 [Zea mays] | 1 | Zea mays | 2635 | 4079 |
| | | | | &gt:gi\|194690250\|gb\|ACF79209.1\| unknown | | | | |
| | | | | [Zea mays] | | | | |
| | | | | &gt:gi\|195636434\|gb\|ACG37685.1\| | | | | |
| | | | | hypothetical protein [Zea mays] | | | | |
| | | | | &gt:gi\|195640568\|gb\|ACG39752.1\| hypothetical | | | | |
| | | | | protein [Zea mays] | | | | |
| | | NP_001144387 | 226531256 | hypothetical protein LOC100277315 [Zea mays] | 0.891304 | Zea mays | 2636 | 4080 |
| | | | | &gt:gi\|195641390\|gb\|ACG40163.1\| hypothetical | | | | |
| | | | | protein [Zea mays] | | | | |
| | | EEE67740 | 54398660 | hypothetical protein OsJ_25431 | 0.791925 | Oryza sativa Japonica Group | 2637 | |
| | | | | [Oryza sativa Japonica Group] | | | | |
| | | EAZ05000 | 54362548 | hypothetical protein OsI_27180 | 0.785714 | Oryza sativa Indica Group | 2638 | |
| | | | | [Oryza sativa Indica Group] | | | | |
| | | BAC15474 | 22775614 | unknown protein [Oryza sativa Japonica Group] | 0.782609 | Oryza sativa Japonica Group | 2639 | 4081 |
| | | | | &gt:gi\|50510136\|dbj\|BAD31101.1\| unknown protein | | | | |
| | | | | [Oryza sativa Japonica Group] | | | | |
| | | BAJ85816 | 326495439 | predicted protein | 0.742236 | Hordeum vulgare subsp. vulgare | 2640 | 4082 |
| | | | | [Hordeum vulgare subsp. vulgare] | | | | |
| | | BAJ87777 | 326513515 | predicted protein | 0.742236 | Hordeum vulgare subsp. vulgare | 2641 | 4083 |
| | | | | [Hordeum vulgare subsp. vulgare] | | | | |
| | 259-280 | XP_002465189 | 242035588 | hypothetical protein SORBIDRAFT_01g033670 | 1 | Sorghum bicolor | 2642 | 4084 |
| | | | | [Sorghum bicolor] | | | | |
| | | | | &gt:gi\|241919043\|gb\|EER92187.1\| | | | | |
| | | | | hypothetical protein SORBIDRAFT_01g033670 | | | | |
| | | | | [Sorghum bicolor] | | | | |
| | 589-610 | AAV64214 | 55741072 | stk [Zea mays] | 1 | Zea mays | 2643 | 4085 |
| | | EEC74143 | 54362548 | hypothetical protein OsI_09217 | 0.707953 | Oryza sativa Indica Group | 2644 | |
| | | | | [Oryza sativa Indica Group] | | | | |
| | | NP_001048340 | 115449120 | Os02g0787200 [Oryza sativa Japonica Group] | 0.706649 | Oryza sativa Japonica Group | 2645 | 4086 |
| | | | | &gt:gi\|47497167\|dbj\|BAD19215.1\| putative serine | | | | |
| | | | | threonine kinase [Oryza sativa Japonica Group] | | | | |
| | | | | &gt:gi\|47497752\|dbj\|BAD19852.1\| putative serine | | | | |
| | | | | threonine kinase [Oryza sativa Japonica Group] | | | | |
| | | | | &gt:gi\|113537871\|dbj\|BAF10254.1\| Os02g0787200 | | | | |
| | | | | [Oryza sativa Japonica Group] | | | | |
| | 89-110 | NP_001147835 | 226528692 | lectin-like receptor kinase 7 [Zea mays] | 1 | Zea mays | 2646 | 4087 |
| | | | | &gt:gi\|195614030\|gb\|ACG28845.1\| lectin-like | | | | |
| | | | | receptor kinase 7 [Zea mays] | | | | |
| | | XP_002463621 | 242032452 | hypothetical protein SORBIDRAFT_01g003030 | 0.860704 | Sorghum bicolor | 2647 | 4088 |
| | | | | [Sorghum bicolor] | | | | |
| | | | | &gt:gi\|241917475\|gb\|EER90619.1\| | | | | |
| | | | | hypothetical protein SORBIDRAFT_01g003030 | | | | |
| | | | | [Sorghum bicolor] | | | | |
| | 169-190 | XP_002467786 | 242040782 | hypothetical protein SORBIDRAFT_01g034030 | 1 | Sorghum bicolor | 2648 | 4089 |
| | | | | [Sorghum bicolor] | | | | |
| | | | | &gt:gi\|241921640\|gb\|EER94784.1\| | | | | |
| | | | | hypothetical protein SORBIDRAFT_01g034030 | | | | |
| | | | | [Sorghum bicolor] | | | | |
| | | NP_001141138 | 226530821 | hypothetical protein LOC100273224 [Zea mays] | 0.903353 | Zea mays | 2649 | 4090 |
| | | | | &gt:gi\|194702834\|gb\|ACF85501.1\| unknown | | | | |
| | | | | [Zea mays] | | | | |
| | | NP_001141473 | 226499929 | hypothetical protein LOC100273583 [Zea mays] | 0.90927 | Zea mays | 2650 | 4091 |
| | | | | &gt:gi\|194704720\|gb\|ACF86444.1\| unknown | | | | |
| | | | | [Zea mays] | | | | |
| | | BAK05639 | 326491078 | predicted protein | 0.745562 | Hordeum vulgare subsp. vulgare | 2651 | 4092 |
| | | | | [Hordeum vulgare subsp. vulgare] | | | | |
| | | NP_001050230 | 115453258 | Os03g0378000 [Oryza sativa Japonica Group] | 0.755424 | Oryza sativa Japonica Group | 2652 | 4093 |
| | | | | &gt:gi\|108708445\|gb\|ABF96240.1\| GDA1/CD39 | | | | |
| | | | | family protein, expressed | | | | |
| | | | | [Oryza sativa Japonica Group] | | | | |
| | | | | &gt:gi\|113548701\|dbj\|BAF12144.1\| | | | | |
| | | | | Os03g0378000 [Oryza sativa Japonica Group] | | | | |
| | | | | &gt:gi\|215704674\|dbj\|BAG94302.1\| unnamed protein | | | | |
| | | | | product [Oryza sativa Japonica Group] | | | | |
| | | EEC75360 | 54362548 | hypothetical protein OsI_11798 | 0.751479 | Oryza sativa Indica Group | 2653 | |
| | | | | [Oryza sativa Indica Group] | | | | |
| | | EEE59145 | 54398660 | hypothetical protein OsJ_11044 | 0.749507 | Oryza sativa Japonica Group | 2654 | |
| | | | | [Oryza sativa Japonica Group] | | | | |
| | 178-199 | XP_002463998 | 242033206 | hypothetical protein SORBIDRAFT_01g010290 | 1 | Sorghum bicolor | 2655 | 4094 |
| | | | | [Sorghum bicolor] | | | | |
| | | | | &gt:gi\|241917852\|gb\|EER90996.1\| | | | | |
| | | | | hypothetical protein SORBIDRAFT_01g010290 | | | | |
| | | | | [Sorghum bicolor] | | | | |
| | | NP_001151626 | 226498593 | atypical receptor-like kinase MARK [Zea mays] | 0.873563 | Zea mays | 2656 | 4095 |
| | | | | &gt:gi\|195648190\|gb\|ACG43563.1\| atypical | | | | |
| | | | | receptor-like kinase MARK [Zea mays] | | | | |
| | | NP_001105207 | 162459880 | atypical receptor-like kinase MARK [Zea mays] | 0.906609 | Zea mays | 2657 | 4096 |
| | | | | &gt:gi\|37778684\|gb\|AAO83390.1\| atypical | | | | |
| | | | | receptor-like kinase MARK [Zea mays] | | | | |
| | | AAK18840 | 13249436 | putative receptor kinase | 0.760057 | Oryza sativa Japonica Group | 2658 | 4097 |
| | | | | [Oryza sativa Japonica Group] | | | | |
| | | | | &gt:gi\|108710729\|gb\|ABF98524.1\| atypical | | | | |
| | | | | receptor-like kinase MARK, putative, expressed | | | | |
| | | | | [Oryza sativa Japonica Group] | | | | |
| | 376-397 | NP_001146675 | 226530240 | hypothetical protein LOC100280275 [Zea mays] | 1 | Zea mays | 2659 | 4098 |
| | | | | &gt:gi\|219888259\|gb\|ACL54504.1\| unknown | | | | |
| | | | | [Zea mays] | | | | |
| | | ACN28169 | 223948170 | unknown [Zea mays] | 0.727083 | Zea mays | 2660 | 4099 |
| | 1237-1258 | NP_001131617 | 239048016 | hypothetical protein LOC100192969 [Zea mays] | 1 | Zea mays | 2661 | 4100 |
| | | | | &gt:gi\|238908597\|gb\|ACF80108.2\| unknown | | | | |
| | | | | [Zea mays] | | | | |
| | | EAZ00403 | 54362548 | hypothetical protein OsI_22418 | 0.869565 | Oryza sativa Indica Group | 2662 | |
| | | | | [Oryza sativa Indica Group] | | | | |
| | | NP_001136728 | 219363410 | hypothetical protein LOC100216867 [Zea mays] | 0.804348 | Zea mays | 2663 | 4101 |
| | | | | &gt:gi\|194699722\|gb\|ACF83945.1\| unknown | | | | |
| | | | | [Zea mays] | | | | |
| | 72-93 | XP_002438795 | 242096609 | hypothetical protein SORBIDRAFT_10g026410 | 1 | Sorghum bicolor | 2664 | 4102 |
| | | | | [Sorghum bicolor] | | | | |
| | | | | &gt:gi\|241917018\|gb\|EER90162.1\| | | | | |
| | | | | hypothetical protein SORBIDRAFT_10g026410 | | | | |
| | | | | [Sorghum bicolor] | | | | |
| | | NP_001146201 | 226533323 | hypothetical protein LOC100279771 [Zea mays] | 0.931174 | Zea mays | 2665 | 4103 |
| | | | | &gt:gi\|219886163\|gb\|ACL53456.1\| unknown | | | | |
| | | | | [Zea mays] | | | | |
| | | EAZ01968 | 54362548 | hypothetical protein OsI_24000 | 0.717949 | Oryza sativa Indica Group | 2666 | |
| | | | | [Oryza sativa Indica Group] | | | | |
| | | BAD45848 | 47169781 | putative microtubule-associated protein | 0.717949 | Oryza sativa Japonica Group | 2667 | 4104 |
| | | | | [Oryza sativa Japonica Group] | | | | |
| | | | | &gt:gi\|52077384\|dbj\|BAD46424.1\| | | | | |
| | | | | putative microtubule-associated protein | | | | |
| | | | | [Oryza sativa Japonica Group] | | | | |
| | | | | &gt:gi\|125598116\|gb\|EAZ37896.1\| hypothetical | | | | |
| | | | | protein OsJ_22246 [Oryza sativa Japonica Group] | | | | |
| | | | | &gt:gi\|215695188\|dbj\|BAG90379.1\| | | | | |
| | | | | unnamed protein product [Oryza sativa Japonica Group] | | | | |
| | 778-799 | NP_001183778 | 308081574 | hypothetical protein LOC100502371 [Zea mays] | 1 | Zea mays | 2668 | 4105 |
| | | | | &gt:gi\|18092335\|gb\|AAL59227.1\|AF448416_5 serine | | | | |
| | | | | threonine kinase [Zea mays] | | | | |
| | 35-56 | XP_002437861 | 242094741 | hypothetical protein SORBIDRAFT_10g003930 | 1 | Sorghum bicolor | 2669 | 4106 |
| | | | | [Sorghum bicolor] | | | | |
| | | | | &gt:gi\|241916084\|gb\|EER89228.1\| | | | | |
| | | | | hypothetical protein SORBIDRAFT_10g003930 | | | | |
| | | | | [Sorghum bicolor] | | | | |
| | | ACN31616 | 223974856 | unknown [Zea mays] | 0.891008 | Zea mays | 2670 | 4107 |
| | | NP_001168581 | 293337218 | hypothetical protein LOC100382365 [Zea mays] | 0.899183 | Zea mays | 2671 | 4108 |
| | | | | &gt:gi\|223944685\|gb\|ACN26426.1\| unknown | | | | |
| | | | | [Zea mays] | | | | |
| | | | | &gt:gi\|223949323\|gb\|ACN28745.1\| | | | | |
| | | | | unknown [Zea mays] | | | | |
| | | EEE65121 | 54398660 | hypothetical protein OsJ_20187 | 0.825613 | Oryza sativa Japonica Group | 2672 | |
| | | | | [Oryza sativa Japonica Group] | | | | |
| | | BAJ90380 | 326494221 | predicted protein [Hordeum vulgare subsp. vulgare] | 0.803815 | Hordeum vulgare subsp. vulgare | 2673 | 4109 |
| | | | | &gt:gi\|326494274\|dbj\|BAJ90406.1\| predicted protein | | | | |
| | | | | [Hordeum vulgare subsp. vulgare] | | | | |
| | | ABL11233 | 118725124 | UCW116, putative lipase | 0.803815 | Hordeum vulgare subsp. vulgare | 2674 | 4110 |
| | | | | [Hordeum vulgare subsp. vulgare] | | | | |
| | | NP_001141565 | 226507421 | hypothetical protein LOC100273681 [Zea mays] | 0.779292 | Zea mays | 2675 | 4111 |
| | | | | &gt:gi\|194705086\|gb\|ACF86627.1\| unknown | | | | |
| | | | | [Zea mays] | | | | |
| | | ACG38031 | 195637125 | alpha-L-fucosidase 2 precursor [Zea mays] | 0.779292 | Zea mays | 2676 | 4112 |
| | | XP_002453668 | 242064757 | hypothetical protein SORBIDRAFT_04g010100 | 0.760218 | Sorghum bicolor | 2677 | 4113 |
| | | | | [Sorghum bicolor] | | | | |
| | | | | &gt:gi\|241933499\|gb\|EES06644.1\| | | | | |
| | | | | hypothetical protein SORBIDRAFT_04g010100 | | | | |
| | | | | [Sorghum bicolor] | | | | |
| | | NP_001056858 | 115466517 | Os06g0157000 [Oryza sativa Japonica Group] | 0.757493 | Oryza sativa Japonica Group | 2678 | 4114 |
| | | | | &gt:gi\|113594898\|dbj\|BAF18772.1\| Os06g0157000 | | | | |
| | | | | [Oryza sativa Japonica Group] | | | | |
| Predicted | 214-235 | NP_001131499 | 212720749 | hypothetical protein LOC100192836 [Zea mays] | 1 | Zea mays | 2679 | 4115 |
| siRNA | | | | &gt:gi\|494691702\|gb\|ACF79935.1\| unknown | | | | |
| 55684 | | | | [Zea mays] | | | | |
| | 275-296 | NP_001150152 | 226497009 | LOC100283781 [Zea mays] | 1 | Zea mays | 2680 | 4116 |
| | | | | &gt:gi\|195637168\|gb\|ACG38052.1\| cathepsin B-like | | | | |
| | | | | cysteine proteinase 3 precursor [Zea mays] | | | | |
| | | ACY38050 | 262217336 | cathepsin B [Dactylis glomerata] | 0.786744 | Dactylis glomerata | 2681 | 4117 |
| | | BAG90919 | 32978477 | unnamed protein product | 0.789625 | Oryza sativa Japonica Group | 2682 | 4118 |
| | | | | [Oryza sativa Japonica Group] | | | | |
| | | CAQ00112 | 194352767 | papain-like cysteine proteinase | 0.743516 | Hordeum vulgare subsp. vulgare | 2683 | 4119 |
| | | | | [Hordeum vulgare subsp. vulgare] | | | | |
| | | | | &gt:gi\|326488519\|dbj\|BAJ93928.1\| predicted | | | | |
| | | | | protein [Hordeum vulgare subsp. vulgare] | | | | |
| | | | | &gt:gi\|326508126\|dbj\|BAJ99330.1\| predicted | | | | |
| | | | | protein [Hordeum vulgare subsp. vulgare] | | | | |
| | | CAC83720 | 40643249 | cathepsin B [Hordeum vulgare subsp. vulgare] | 0.74928 | Hordeum vulgare subsp. vulgare | 2684 | 4120 |
| | | | | &gt:gi\|326494236\|dbj\|BAJ90387.1\| predicted | | | | |
| | | | | protein [Hordeum vulgare subsp. vulgare] | | | | |
| | | | | &gt:gi\|326499864\|dbj\|BAJ90767.1\| predicted | | | | |
| | | | | protein [Hordeum vulgare subsp. vulgare] | | | | |
| | | BAJ90118 | 326490901 | predicted protein [Hordeum vulgare subsp. vulgare] | 0.737752 | Hordeum vulgare subsp. vulgare | 2685 | 4121 |
| | | | | &gt:gi\|326508404\|dbj\|BAJ99469.1\| predicted | | | | |
| | | | | protein [Hordeum vulgare subsp. vulgare] | | | | |
| | | | | &gt:gi\|326514912\|dbj\|BAJ99817.1\| predicted | | | | |
| | | | | protein [Hordeum vulgare subsp. vulgare] | | | | |
| | | CAA46810 | 21692 | cathepsin B [Triticum aestivum] | 0.73487 | Triticum aestivum | 2686 | 4122 |
| | | CAA46811 | 21698 | cathepsin B [Triticum aestivum] | 0.717579 | Triticum aestivum | 2687 | 4123 |
| | 260-281 | NP_001146149 | 226505901 | hypothetical protein LOC100279718 [Zea mays] | 1 | Zea mays | 2688 | 4124 |
| | | | | &gt:gi\|219885973\|gb\|ACL53361.1\| unknown | | | | |
| | | | | [Zea mays] | | | | |
| | | XP_002463310 | 242051131 | hypothetical protein SORBIDRAFT_02g041560 | 0.802419 | Sorghum bicolor | 2689 | 4125 |
| | | | | [Sorghum bicolor] | | | | |
| | | | | &gt:gi\|241926687\|gb\|EER99831.1\| | | | | |
| | | | | hypothetical protein SORBIDRAFT_02g041560 | | | | |
| | | | | [Sorghum bicolor] | | | | |
| | 654-675 | NP_001143915 | 226500557 | hypothetical protein LOC100276723 [Zea mays] | 1 | Zea mays | 2690 | 4126 |
| | | | | &gt:gi\|195629462\|gb\|ACG36372.1\| hypothetical | | | | |
| | | | | protein [Zea mays] | | | | |
| | | XP_002458638 | 242058984 | hypothetical protein SORBIDRAFT_03g037170 | 0.948171 | Sorghum bicolor | 2691 | 4127 |
| | | | | [Sorghum bicolor] | | | | |
| | | | | &gt:gi\|241930613\|gb\|EES03758.1\| | | | | |
| | | | | hypothetical protein SORBIDRAFT_03g037170 | | | | |
| | | | | [Sorghum bicolor] | | | | |
| | | NP_001144570 | 226528574 | hypothetical protein LOC100277579 [Zea mays] | 0.838415 | Zea mays | 2692 | 4128 |
| | | | | &gt:gi\|195643964\|gb\|ACG41450.1\| hypothetical | | | | |
| | | | | protein [Zea mays] | | | | |
| | | EAY76156 | 54362548 | hypothetical protein OsI_04089 | 0.844512 | Oryza sativa Indica Group | 2693 | |
| | | | | [Oryza sativa Indica Group] | | | | |
| | | NP_001044532 | 115440504 | Os01g0800300 [Oryza sativa Japonica Group] | 0.844512 | Oryza sativa Japonica Group | 2694 | 4129 |
| | | | | &gt:gi\|19570984\|dbj\|BAB86411.1\| unknown | | | | |
| | | | | protein [Oryza sativa Japonica Group] | | | | |
| | | | | &gt:gi\|20804736\|dbj\|BAB92422.1\| unknown | | | | |
| | | | | protein [Oryza sativa Japonica Group] | | | | |
| | | | | &gt:gi\|113534063\|dbj\|BAF06446.1\| Os01g0800300 | | | | |
| | | | | [Oryza sativa Japonica Group] | | | | |
| | | | | &gt:gi\|215704187\|dbj\|BAG93027.1\| unnamed protein | | | | |
| | | | | product [Oryza sativa Japonica Group] | | | | |
| | | | | &gt:gi\|215704815\|dbj\|BAG94843.1\| unnamed protein | | | | |
| | | | | product [Oryza sativa Japonica Group] | | | | |
| | | | | &gt:gi\|215704819\|dbj\|BAG94847.1\| unnamed protein | | | | |
| | | | | product [Oryza sativa Japonica Group] | | | | |
| | | | | &gt:gi\|215741020\|dbj\|BAG97515.1\| unnamed protein | | | | |
| | | | | product [Oryza sativa Japonica Group] | | | | |
| | | BAK06822 | 326513163 | predicted protein | 0.783537 | Hordeum vulgare subsp. vulgare | 2695 | 4130 |
| | | | | [Hordeum vulgare subsp. vulgare] | | | | |
| | | XP_002457313 | 242056334 | hypothetical protein SORBIDRAFT_03g005440 | 0.759146 | Sorghum bicolor | 2696 | 4131 |
| | | | | [Sorghum bicolor] | | | | |
| | | | | &gt:gi\|241929288\|gb\|EES02433.1\| | | | | |
| | | | | hypothetical protein SORBIDRAFT_03g005440 | | | | |
| | | | | [Sorghum bicolor] | | | | |
| | 13-34 | XP_002465221 | 242035652 | hypothetical protein SORBIDRAFT_01g034400 | 1 | Sorghum bicolor | 2697 | 4132 |
| | | | | [Sorghum bicolor] | | | | |
| | | | | &gt:gi\|241919075\|gb\|EER92219.1\| | | | | |
| | | | | hypothetical protein SORBIDRAFT_01g034400 | | | | |
| | | | | [Sorghum bicolor] | | | | |
| | | ACF85210 | 194702251 | unknown [Zea mays] | 0.970696 | Zea mays | 2698 | 4133 |
| | | | | &gt:gi\|223942479\|gb\|ACN25323.1\| | | | | |
| | | | | unknown [Zea mays] | | | | |
| | | NP_001050195 | 115453188 | Os03g0369800 [Oryza sativa Japonica Group] | 0.879121 | Oryza sativa Japonica Group | 2699 | 4134 |
| | | | | &gt:gi\|12039356\|gb\|AAG46143.1\|AC082644_25 | | | | |
| | | | | putative vesicle soluble NSF attachment protein | | | | |
| | | | | receptor [Oryza sativa Japonica Group] | | | | |
| | | | | &gt:gi\|108708370\|gb\|ABF96165.1\| Novel plant | | | | |
| | | | | SNARE 11, putative, expressed | | | | |
| | | | | [Oryza sativa Japonica Group] | | | | |
| | | | | &gt:gi\|113548666\|dbj\|BAF12109.1\| | | | | |
| | | | | Os03g0369800 [Oryza sativa Japonica Group] | | | | |
| | | | | &gt:gi\|125586400\|gb\|EAZ27064.1\| hypothetical | | | | |
| | | | | protein OsJ_10993 [Oryza sativa Japonica Group] | | | | |
| | | | | &gt:gi\|215694052\|dbj\|BAG89251.1\| unnamed protein | | | | |
| | | | | product [Oryza sativa Japonica Group] | | | | |
| | | EAY90177 | 54362548 | hypothetical protein OsI_11742 | 0.879121 | Oryza sativa Indica Group | 2700 | |
| | | | | [Oryza sativa Indica Group] | | | | |
| | | AAU94636 | 53829386 | SNARE 12 [Oryza sativa Japonica Group] | 0.875458 | Oryza sativa Japonica Group | 2701 | 4135 |
| | | EEE67665 | 54398660 | hypothetical protein OsJ_25287 | 0.791209 | Oryza sativa Japonica Group | 2702 | |
| | | | | [Oryza sativa Japonica Group] | | | | |
| | | XP_002461096 | 242046449 | hypothetical protein SORBIDRAFT_02g040620 | 0.802198 | Sorghum bicolor | 2703 | 4136 |
| | | | | [Sorghum bicolor] | | | | |
| | | | | &gt:gi\|241924473\|gb\|EER97617.1\| | | | | |
| | | | | hypothetical protein SORBIDRAFT_02g040620 | | | | |
| | | | | [Sorghum bicolor] | | | | |
| | | NP_001151516 | 226502489 | novel plant SNARE 11 [Zea mays] | 0.798535 | Zea mays | 2704 | 4137 |
| | | | | &gt:gi\|195647358\|gb\|ACG43147.1\| | | | | |
| | | | | novel plant SNARE 11 [Zea mays] | | | | |
| | | | | &gt:gi\|219887387\|gb\|ACL54068.1\| | | | | |
| | | | | unknown [Zea mays] | | | | |
| | | NP_001060402 | 115473606 | Os07g0637400 [Oryza sativa Japonica Group] | 0.791209 | Oryza sativa Japonica Group | 2705 | 4138 |
| | | | | &gt:gi\|23237830\|dbj\|BAC16405.1\| vesicle soluble | | | | |
| | | | | NSF attachment protein receptor-like | | | | |
| | | | | protein [Oryza sativa Japonica Group] | | | | |
| | | | | &gt:gi\|113611938\|dbj\|BAF22316.1\| Os07g0637400 | | | | |
| | | | | [Oryza sativa Japonica Group] | | | | |
| | | | | &gt:gi\|215695299\|dbj\|BAG90490.1\| unnamed protein | | | | |
| | | | | product [Oryza sativa Japonica Group] | | | | |
| | | AAU94637 | 53829388 | SNARE 13 [Oryza sativa Japonica Group] | 0.791209 | Oryza sativa Japonica Group | 2706 | 4139 |
| | 295-316 | AAX11351 | 59895950 | cathepsin B-like cysteine protease | 1 | Oryza sativa Japonica Group | 2707 | 4140 |
| | | | | [Oryza sativa Japonica Group] | | | | |
| | | | | &gt:gi\|125551767\|gb\|EAY97476.1\| | | | | |
| | | | | hypothetical protein OsI_19406 | | | | |
| | | | | [Oryza sativa Indica Group] | | | | |
| | | | | &gt:gi\|215694023\|dbj\|BAG89222.1\| unnamed | | | | |
| | | | | protein product [Oryza sativa Japonica Group] | | | | |
| | | | | &gt:gi\|215712372\|dbj\|BAG94499.1\| unnamed | | | | |
| | | | | protein product [Oryza sativa Japonica Group] | | | | |
| | | | | &gt:gi\|215765382\|dbj\|BAG87079.1\| unnamed | | | | |
| | | | | protein product [Oryza sativa Japonica Group] | | | | |
| | | | | &gt:gi\|222631058\|gb\|EEE63190.1\| hypothetical | | | | |
| | | | | protein OsJ_17999 [Oryza sativa Japonica Group] | | | | |
| | | BAJ90198 | 326492683 | predicted protein | 0.726257 | Hordeum vulgare subsp. vulgare | 2708 | 4141 |
| | | | | [Hordeum vulgare subsp. vulgare] | | | | |
| | 554-575 | NP_001170197 | 293334350 | hypothetical protein LOC100384147 [Zea mays] | 1 | Zea mays | 2709 | 4142 |
| | | | | &gt:gi\|224034241\|gb\|ACN36196.1\| unknown | | | | |
| | | | | [Zea mays] | | | | |
| | 526-547 | NP_001106022 | 162461199 | putative growth-regulating factor 3 [Zea mays] | 1 | Zea mays | 2710 | 4143 |
| | | | | &gt:gi\|146008369\|gb\|ABQ01216.1\| putative | | | | |
| | | | | growth-regulating factor 3 [Zea mays] | | | | |
| | | ACG42410 | 195645883 | atGRF5 [Zea mays] | 0.9825 | Zea mays | 2711 | 4144 |
| | | XP_002454651 | 242066723 | hypothetical protein SORBIDRAFT_04g034800 | 0.805 | Sorghum bicolor | 2712 | 4145 |
| | | | | [Sorghum bicolor] | | | | |
| | | | | &gt:gi\|241934482\|gb\|EES07627.1\| | | | | |
| | | | | hypothetical protein SORBIDRAFT_04g034800 | | | | |
| | | | | [Sorghum bicolor] | | | | |
| | | NP_001048288 | 115449016 | Os02g0776900 [Oryza sativa Japonica Group] | 0.725 | Oryza sativa Japonica Group | 2713 | 4146 |
| | | | | &gt:gi\|51039831\|tpg\|DAA05205.1\| TPA_exp: | | | | |
| | | | | growth-regulating factor 1 | | | | |
| | | | | [Oryza sativa (japonica cultivar-group)] | | | | |
| | | | | &gt:gi\|113537819\|dbj\|BAF10202.1\| | | | | |
| | | | | Os02g0776900 [Oryza sativa Japonica Group] | | | | |
| | | AAF17567 | 6573148 | growth-regulating factor 1 [Oryza sativa] | 0.7275 | Oryza sativa | 2714 | 4147 |
| | | | | &gt:gi\|125541338\|gb\|EAY87733.1\| hypothetical | | | | |
| | | | | protein OsI_09149 [Oryza sativa Indica Group] | | | | |
| | | NP_001106027 | 162461967 | putative growth-regulating factor 9 [Zea mays] | 0.7125 | Zea mays | 2715 | 4148 |
| | | | | &gt:gi\|146008476\|gb\|ABQ01222.1\| putative | | | | |
| | | | | growth-regulating factor 9 [Zea mays] | | | | |
| | 257-278 | XP_002465189 | 242035588 | hypothetical protein SORBIDRAFT_01g033670 | 1 | Sorghum bicolor | 2716 | 4149 |
| | | | | [Sorghum bicolor] | | | | |
| | | | | &gt:gi\|241919043\|gb\|EER92187.1\| | | | | |
| | | | | hypothetical protein SORBIDRAFT_01g033670 | | | | |
| | | | | [Sorghum bicolor] | | | | |
| Predicted | 247-270 | NP_001105185 | 162463524 | sigma factor protein [Zea mays] | 1 | Zea mays | 2717 | 4150 |
| siRNA | | | | &gt:gi\|20159761\|gb\|AAM12034.1\| | | | | |
| 56658 | | | | sigma factor protein [Zea mays] | | | | |
| | | ACN32028 | 223975680 | unknown [Zea mays] | 0.924296 | Zea mays | 2718 | 4151 |
| | | XP_002440362 | 242089058 | hypothetical protein SORBIDRAFT_09g030350 | 0.727113 | Sorghum bicolor | 2719 | 4152 |
| | | | | [Sorghum bicolor] | | | | |
| | | | | &gt:gi\|241945647\|gb\|EES18792.1\| | | | | |
| | | | | hypothetical protein SORBIDRAFT_09g030350 | | | | |
| | | | | [Sorghum bicolor] | | | | |
| Predicted | 170-191 | NP_001147862 | 226497613 | LOC100281472 [Zea mays] | 1 | Zea mays | 2720 | 4153 |
| siRNA | | | | &gt:gi\|195614188\|gb\|ACG28924.1\| | | | | |
| 56885 | | | | transparent testa 12 protein [Zea mays] | | | | |
| | | BAJ87592 | 326511155 | predicted protein [Hordeum vulgare subsp. vulgare] | 0.778468 | Hordeum vulgare subsp. vulgare | 2721 | 4154 |
| | | | | &gt:gi\|326521392\|dbj\|BAJ96899.1\| predicted | | | | |
| | | | | protein [Hordeum vulgare subsp. vulgare] | | | | |
| | 483-504 | XP_002467897 | 242041004 | hypothetical protein SORBIDRAFT_01g036050 | 1 | Sorghum bicolor | 2722 | 4155 |
| | | | | [Sorghum bicolor] | | | | |
| | | | | &gt:gi\|241921751\|gb\|EER94895.1\| | | | | |
| | | | | hypothetical protein SORBIDRAFT_01g036050 | | | | |
| | | | | [Sorghum bicolor] | | | | |
| | | NP_001146010 | 226508499 | hypothetical protein LOC100279541 [Zea mays] | 0.945342 | Zea mays | 2723 | 4156 |
| | | | | &gt:gi\|219885317\|gb\|ACL53033.1\| | | | | |
| | | | | unknown [Zea mays] | | | | |
| | | | | &gt:gi\|223944401\|gb\|ACN26284.1\| | | | | |
| | | | | unknown [Zea mays] | | | | |
| | | Q8VXB5 | | RecName: Full = Putative potassium transporter 8; | 0.880745 | Oryza sativa Japonica Group | 2724 | |
| | | | | AltName: Full = OsHAK8 | | | | |
| | | | | &gt:gi\|108708033\|gb\|ABF95828.1\| | | | | |
| | | | | Potassium transporter 2, putative, expressed | | | | |
| | | | | [Oryza sativa Japonica Group] | | | | |
| | | | | &gt:gi\|125586181\|gb\|EAZ26845.1\| | | | | |
| | | | | hypothetical protein OsJ_10761 | | | | |
| | | | | [Oryza sativa Japonica Group] | | | | |
| | | EAY89924 | 54362548 | hypothetical protein OsI_11472 | 0.879503 | Oryza sativa Indica Group | 2725 | |
| | | | | [Oryza sativa Indica Group] | | | | |
| | | NP_001060637 | 115474076 | Os07g0679000 [Oryza sativa Japonica Group] | 0.807453 | Oryza sativa Japonica Group | 2726 | 4157 |
| | | | | &gt:gi\|75232649\|sp\|Q7XIV8.1\|HAK9_ORYSJ | | | | |
| | | | | RecName: Full = Probable potassium transporter 9; | | | | |
| | | | | AltName: Full = OsHAK9 | | | | |
| | | | | &gt:gi\|18250702\|emb\|CAD20999.1\| putative potasium | | | | |
| | | | | transporter [Oryza sativa Japonica Group] | | | | |
| | | | | &gt:gi\|33146437\|dbj\|BAC79545.1\| | | | | |
| | | | | putative potassium transporter | | | | |
| | | | | [Oryza sativa Japonica Group] | | | | |
| | | | | &gt:gi\|113612173\|dbj\|BAF2255.1\| Os07g0679000 | | | | |
| | | | | [Oryza sativa Japonica Group] | | | | |
| | | | | &gt:gi\|125559610\|gb\|EAZ05146.1\| hypothetical | | | | |
| | | | | protein OsI_27340 [Oryza sativa Indica Group] | | | | |
| | | BAE93160 | 91204711 | potassium transporter [Phragmites australis] | 0.804969 | Phragmites australis | 2727 | 4158 |
| | | BAE93159 | 91204709 | potassium transporter [Phragmites australis] | 0.803727 | Phragmites australis | 2728 | 4159 |
| | | BAE93158 | 91204707 | potassium transporter [Phragmites australis] | 0.807453 | Phragmites australis | 2729 | 4160 |
| | | EAZ41092 | 54398660 | hypothetical protein OsJ_25584 | 0.781366 | Oryza sativa Japonica Group | 2730 | |
| | | | | [Oryza sativa Japonica Group] | | | | |
| | | BAJ87873 | 326513707 | predicted protein [Hordeum vulgare subsp. vulgare] | 0.762733 | Hordeum vulgare subsp. vulgare | 2731 | 4161 |
| | | | | &gt:gi\|326525935\|dbj\|BAJ93144.1\| predicted | | | | |
| | | | | protein [Hordeum vulgare subsp. vulgare] | | | | |
| | 378-399 | XP_002438701 | 242096421 | hypothetical protein SORBIDRAFT_10g024660 | 1 | Sorghum bicolor | 2732 | 4162 |
| | | | | [Sorghum bicolor] | | | | |
| | | | | &gt:gi\|241916924\|gb\|EER90068.1\| | | | | |
| | | | | hypothetical protein SORBIDRAFT_10g024660 | | | | |
| | | | | [Sorghum bicolor] | | | | |
| | | NP_001147472 | 226504515 | potassium transporter 10 [Zea mays] | 0.937805 | Zea mays | 2733 | 4163 |
| | | | | &gt:gi\|195611632\|gb\|ACG27646.1\| | | | | |
| | | | | potassium transporter 10 [Zea mays] | | | | |
| | | BAK03495 | 326515163 | predicted protein | 0.831707 | Hordeum vulgare subsp. vulgare | 2734 | 4164 |
| | | | | [Hordeum vulgare subsp. vulgare] | | | | |
| | | CAD21000 | 18250703 | putative potasium transporter | 0.820732 | Oryza sativa Japonica Group | 2735 | 4165 |
| | | | | [Oryza sativa Japonica Group] | | | | |
| | | NP_001058116 | 115469033 | Os06g0625900 [Oryza sativa Japonica Group] | 0.828049 | Oryza sativa Japonica Group | 2736 | 4166 |
| | | | | &gt:gi\|62900352\|sp\|Q67VS5.1\|HAK10_ORYSJ | | | | |
| | | | | RecName: Full = Potassium transporter 10; | | | | |
| | | | | AltName: Full = OsHAK10 | | | | |
| | | | | &gt:gi\|18250690\|emb\|CAD20993.1\| | | | | |
| | | | | putative potasium transporter | | | | |
| | | | | [Oryza sativa Japonica Group] | | | | |
| | | | | &gt:gi\|51535727\|dbj\|BAD37744.1\| | | | | |
| | | | | putative potassium transporter KUP3p | | | | |
| | | | | [Oryza sativa Japonica Group] | | | | |
| | | | | &gt:gi\|113596156\|dbj\|BAF20030.1\| | | | | |
| | | | | Os06g0625900 [Oryza sativa Japonica Group] | | | | |
| | | CAN75895 | 123699834 | hypothetical protein VITISV_038658 | 0.7 | Vitis vinifera | 2737 | |
| | | | | [Vitis vinifera] | | | | |
| | 402-423 | XP_002463387 | 242051285 | hypothetical protein SORBIDRAFT_02g042930 | 1 | Sorghum bicolor | 2738 | 4167 |
| | | | | [Sorghum bicolor] | | | | |
| | | | | &gt:gi\|241926764\|gb\|EER99908.1\| | | | | |
| | | | | hypothetical protein SORBIDRAFT_02g042930 | | | | |
| | | | | [Sorghum bicolor] | | | | |
| | | NP_001141423 | 226503931 | hypothetical protein LOC100273533 [Zea mays] | 0.934343 | Zea mays | 2739 | 4168 |
| | | | | &gt:gi\|194704534\|gb\|ACF86351.1\| | | | | |
| | | | | unknown [Zea mays] | | | | |
| | | | | &gt:gi\|223945057\|gb\|ACN26612.1\| | | | | |
| | | | | unknown [Zea mays] | | | | |
| | | | | &gt:gi\|223948037\|gb\|ACN28102.1\| | | | | |
| | | | | unknown [Zea mays] | | | | |
| | 31-52 | ABA95869 | 108862058 | protein kinase family protein, putative, expressed | 1 | Oryza sativa Japonica Group | 2740 | |
| | | | | [Oryza sativa Japonica Group] | | | | |
| | | | | &gt:gi\|215769321\|dbj\|BAH01550.1\| unnamed protein | | | | |
| | | | | product [Oryza sativa Japonica Group] | | | | |
| | | EEE52818 | 54398660 | hypothetical protein OsJ_35327 | 0.956848 | Oryza sativa Japonica Group | 2741 | |
| | | | | [Oryza sativa Japonica Group] | | | | |
| | | EEC68910 | 54362548 | hypothetical protein OsI_37582 | 0.840525 | Oryza sativa Indica Group | 2742 | |
| | | | | [Oryza sativa Indica Group] | | | | |
| | | NP_001151569 | 226498197 | ATP binding protein [Zea mays] | 0.840525 | Zea mays | 2743 | 4169 |
| | | | | &gt:gi\|195647818\|gb\|ACG43377.1\| | | | | |
| | | | | ATP binding protein [Zea mays] | | | | |
| | | ACR35824 | 238009577 | unknown [Zea mays] | 0.838649 | Zea mays | 2744 | 4170 |
| | | NP_001141661 | 239047296 | hypothetical protein LOC100273787 [Zea mays] | 0.836773 | Zea mays | 2745 | 4171 |
| | | | | &gt:gi\|238908867\|gb\|ACF86813.2\| | | | | |
| | | | | unknown [Zea mays] | | | | |
| | | BAJ91271 | 326506063 | predicted protein [Hordeum vulgare subsp. vulgare] | 0.78424 | Hordeum vulgare subsp. vulgare | 2746 | 4172 |
| | | BAJ88388 | 326522683 | predicted protein [Hordeum vulgare subsp. vulgare] | 0.78424 | Hordeum vulgare subsp. vulgare | 2747 | 4173 |
| | | NP_001152374 | 226504771 | ATP binding protein [Zea mays] | 0.705441 | Zea mays | 2748 | 4174 |
| | | | | &gt:gi\|195655683\|gb\|ACG47309.1\| | | | | |
| | | | | ATP binding protein [Zea mays] | | | | |
| | | XP_002449038 | 242067522 | hypothetical protein SORBIDRAFT_05g003840 | 0.716698 | Sorghum bicolor | 2749 | 4175 |
| | | | | [Sorghum bicolor] | | | | |
| | | | | &gt:gi\|241934881\|gb\|EES08026.1\| | | | | |
| | | | | hypothetical protein SORBIDRAFT_05g003840 | | | | |
| | | | | [Sorghum bicolor] | | | | |
| | 819-840 | EEE68876 | 54398660 | hypothetical protein OsJ_27688 | 1 | Oryza sativa Japonica Group | 2750 | |
| | | | | [Oryza sativa Japonica Group] | | | | |
| | | EEC83753 | 54362548 | hypothetical protein OsI_29621 | 0.997436 | Oryza sativa Indica Group | 2751 | |
| | | | | [Oryza sativa Indica Group] | | | | |
| | 132-153 | XP_002441141 | 242090616 | hypothetical protein SORBIDRAFT_09g021160 | 1 | Sorghum bicolor | 2752 | 4176 |
| | | | | [Sorghum bicolor] | | | | |
| | | | | &gt:gi\|241946426\|gb\|EES19571.1\| | | | | |
| | | | | hypothetical protein SORBIDRAFT_09g021160 | | | | |
| | | | | [Sorghum bicolor] | | | | |
| | | NP_001105120 | 162461887 | potassium channel protein ZMK2 [Zea mays] | 0.906921 | Zea mays | 2753 | 4177 |
| | | | | &gt:gi\|5830781\|emb\|CAB54856.1\| potassium | | | | |
| | | | | channel protein ZMK2 [Zea mays] | | | | |
| | | NP_001147796 | 226503366 | potassium channel AKT2/3 [Zea mays] | 0.896181 | Zea mays | 2754 | 4178 |
| | | | | &gt:gi\|195613792\|gb\|ACG28726.1\| potassium | | | | |
| | | | | channel AKT2/3 [Zea mays] | | | | |
| | | XP_002441145 | 242090624 | hypothetical protein SORBIDRAFT_09g021210 | 0.872315 | Sorghum bicolor | 2755 | 4179 |
| | | | | [Sorghum bicolor] | | | | |
| | | | | &gt:gi\|241946430\|gb\|EES19575.1\| | | | | |
| | | | | hypothetical protein SORBIDRAFT_09g021210 | | | | |
| | | | | [Sorghum bicolor] | | | | |
| | | XP_002441143 | 242090620 | hypothetical protein SORBIDRAFT_09g021190 | 0.874702 | Sorghum bicolor | 2756 | 4180 |
| | | | | [Sorghum bicolor] | | | | |
| | | | | &gt:gi\|241946428\|gb\|EES19573.1\| | | | | |
| | | | | hypothetical protein SORBIDRAFT_09g021190 | | | | |
| | | | | [Sorghum bicolor] | | | | |
| | | Q75HP9 | | RecName: Full = Potassium channel AKT2 | 0.791169 | Oryza sativa Japonica Group | 2757 | |
| | | | | &gt:gi\|46391141\|gb\|AAS90668.1\| putative potassium | | | | |
| | | | | channel protein [Oryza sativa Japonica Group] | | | | |
| | | | | &gt:gi\|222631670\|gb\|EEE63802.1\| hypothetical | | | | |
| | | | | protein OsJ_18626 [Oryza sativa Japonica Group] | | | | |
| | | EAY98139 | 54362548 | hypothetical protein OsI_20054 | 0.791169 | Oryza sativa Indica Group | 2758 | |
| | | | | [Oryza sativa Indica Group] | | | | |
| | | ABE99811 | 93138732 | inwardly rectifying potassium channel | 0.74821 | Hordeum vulgare | 2759 | 4181 |
| | | | | AKT2 [Hordeum vulgare] | | | | |
| | | | | &gt:gi\|326499398\|dbj\|BAJ86010.1\| predicted | | | | |
| | | | | protein [Hordeum vulgare subsp. vulgare] | | | | |
| | | BAJ86681 | 326507875 | predicted protein | 0.747017 | Hordeum vulgare subsp. vulgare | 2760 | 4182 |
| | | | | [Hordeum vulgare subsp. vulgare] | | | | |
| | | BAJ96949 | 326523876 | predicted protein | 0.747017 | Hordeum vulgare subsp. vulgare | 2761 | 4183 |
| | | | | [Hordeum vulgare subsp. vulgare] | | | | |
| | 1374-1395 | NP_001062056 | 115476919 | Os08g0480000 [Oryza sativa Japonica Group] | 1 | Oryza sativa Japonica Group | 2762 | 4184 |
| | | | | &gt:gi\|42408579\|dbj\|BAD09756.1\| putative ripening | | | | |
| | | | | regulated protein [Oryza sativa Japonica Group] | | | | |
| | | | | &gt:gi\|113624025\|dbj\|BAF23970.1\| Os08g0480000 | | | | |
| | | | | [Oryza sativa Japonica Group] | | | | |
| | | | | &gt:gi\|215695384\|dbj\|BAG90575.1\| unnamed protein | | | | |
| | | | | product [Oryza sativa Japonica Group] | | | | |
| Predicted | 213-230 | ACR33787 | 237908822 | cytochrome P450 monooxygenase [Zea mays] | 1 | Zea mays | 2763 | 4185 |
| siRNA | | | | | | | | |
| 57689 | | | | | | | | |
| | | XP_002456213 | 242054134 | hypothetical protein SORBIDRAFT_03g032220 | 0.790262 | Sorghum bicolor | 2764 | 4186 |
| | | | | [Sorghum bicolor] | | | | |
| | | | | &gt:gi\|241928188\|gb\|EES01333.1\| | | | | |
| | | | | hypothetical protein SORBIDRAFT_03g032220 | | | | |
| | | | | [Sorghum bicolor] | | | | |
| | | EAY75509 | 54362548 | hypothetical protein OsI_03409 | 0.70412 | Oryza sativa Indica Group | 2765 | |
| | | | | [Oryza sativa Indica Group] | | | | |
| | | NP_001172524 | 297720324 | Os01g0701300 [Oryza sativa Japonica Group] | 0.705993 | Oryza sativa Japonica Group | 2766 | 4187 |
| | | | | &gt:gi\|255673594\|dbj\|BAH91254.1\| Os01g0701300 | | | | |
| | | | | [Oryza sativa Japonica Group] | | | | |
| | 809-826 | XP_002439318 | 242086970 | hypothetical protein SORBIDRAFT_09g004290 | 1 | Sorghum bicolor | 2767 | 4188 |
| | | | | [Sorghum bicolor] | | | | |
| | | | | &gt:gi\|241944603\|gb\|EES17748.1\| | | | | |
| | | | | hypothetical protein SORBIDRAFT_09g004290 | | | | |
| | | | | [Sorghum bicolor] | | | | |
| | | NP_001132852 | 212721551 | hypothetical protein LOC100194344 [Zea mays] | 0.989362 | Zea mays | 2768 | 4189 |
| | | | | &gt:gi\|195606262\|gb\|ACG24961.1\| | | | | |
| | | | | 60S ribosomal protein L18 [Zea mays] | | | | |
| | | | | &gt:gi\|195625914\|gb\|ACG34787.1\| | | | | |
| | | | | 60S ribosomal protein L18 [Zea mays] | | | | |
| | | | | &gt:gi\|195640242\|gb\|ACG39589.1\| | | | | |
| | | | | 60S ribosomal protein L18 [Zea mays] | | | | |
| | | | | &gt:gi\|238006204\|gb\|ACR34137.1\| unknown | | | | |
| | | | | [Zea mays] | | | | |
| | | NP_001054688 | 115462176 | Os05g0155100 [Oryza sativa Japonica Group] | 0.968085 | Oryza sativa Japonica Group | 2769 | 4190 |
| | | | | &gt:gi\|54291850\|gb\|AAV32218.1\| | | | | |
| | | | | cytoplasmic ribosomal protein L18 | | | | |
| | | | | [Oryza sativa Japonica Group] | | | | |
| | | | | &gt:gi\|113578239\|dbj\|BAF16602.1\| Os05g0155100 | | | | |
| | | | | [Oryza sativa Japonica Group] | | | | |
| | | | | &gt:gi\|125550901\|gb\|EAY96610.1\| hypothetical | | | | |
| | | | | protein OsI_18515 [Oryza sativa Indica Group] | | | | |
| | | | | &gt:gi\|149392184\|gb\|ABR25945.1\| | | | | |
| | | | | 60S ribosomal protein 118 | | | | |
| | | | | [Oryza sativa Indica Group] | | | | |
| | | | | &gt:gi\|215692755\|dbj\|BAG88175.1\| unnamed protein | | | | |
| | | | | product [Oryza sativa Japonica Group] | | | | |
| | | | | &gt:gi\|215767982\|dbj\|BAH00211.1\| unnamed protein | | | | |
| | | | | product [Oryza sativa Japonica Group] | | | | |
| | | | | &gt:gi\|222630251\|gb\|EEE62383.1\| hypothetical | | | | |
| | | | | protein OsJ_17172 [Oryza sativa Japonica Group] | | | | |
| | | BAJ90421 | 326494303 | predicted protein | 0.925532 | Hordeum vulgare subsp. vulgare | 2770 | 4191 |
| | | | | [Hordeum vulgare subsp. vulgare] | | | | |
| | | | | &gt:gi\|326527807\|dbj\|BAJ88976.1\| predicted | | | | |
| | | | | protein [Hordeum vulgare subsp. vulgare] | | | | |
| | | 3IZ5_R | | Chain R, Localization Of The Large Subunit | 0.920213 | Triticum aestivum | 2771 | |
| | | | | Ribosomal Proteins Into A 5.5 A Cryo-Em Map Of | | | | |
| | | | | Triticum Aestivum Translating 80s Ribosome | | | | |
| | | | | &gt:gi\|315113257\|pdb\|3IZR\|R Chain R, Localization | | | | |
| | | | | Of The Large Subunit Ribosomal Proteins Into A 5.5 | | | | |
| | | | | A Cryo-Em Map Of Triticum Aestivum Translating | | | | |
| | | | | 80s Ribosome &gt:gi\|57471708\|gb\|AAW50985.1\| | | | | |
| | | | | ribosomal protein L18 [Triticum aestivum] | | | | |
| | | XP_002461201 | 242046909 | hypothetical protein SORBIDRAFT_02g042750 | 0.93617 | Sorghum bicolor | 2772 | 4192 |
| | | | | [Sorghum bicolor] | | | | |
| | | | | &gt:gi\|241924578\|gb\|EER97722.1\| | | | | |
| | | | | hypothetical protein SORBIDRAFT_02g042750 | | | | |
| | | | | [Sorghum bicolor] | | | | |
| | | XP_002465302 | 242035814 | hypothetical protein SORBIDRAFT_01g035860 | 0.941489 | Sorghum bicolor | 2773 | 4193 |
| | | | | [Sorghum bicolor] | | | | |
| | | | | &gt:gi\|241919156\|gb\|EER92300.1\| | | | | |
| | | | | hypothetical protein SORBIDRAFT_01g035860 | | | | |
| | | | | [Sorghum bicolor] | | | | |
| | | NP_001131538 | 212721317 | hypothetical protein LOC100192878 [Zea mays] | 0.93617 | Zea mays | 2774 | 4194 |
| | | | | &gt:gi\|195606022\|gb\|ACG24841.1\| | | | | |
| | | | | 60S ribosomal protein L18 [Zea mays] | | | | |
| | | | | &gt:gi\|195620212\|gb\|ACG31936.1\| | | | | |
| | | | | 60S ribosomal protein L18 [Zea mays] | | | | |
| | | NP_001050069 | 115452936 | Os03g0341100 [Oryza sativa Japonica Group] | 0.909574 | Oryza sativa Japonica Group | 2775 | 4195 |
| | | | | &gt:gi\|108708066\|gb\|ABF95861.1\| | | | | |
| | | | | 60S ribosomal protein L18, putative, expressed | | | | |
| | | | | [Oryza sativa Japonica Group] | | | | |
| | | | | &gt:gi\|113548540\|dbj\|BAF11983.1\| | | | | |
| | | | | Os03g0341100 [Oryza sativa Japonica Group] | | | | |
| | | | | &gt:gi\|218192805\|gb\|EEC75232.1\| hypothetical | | | | |
| | | | | protein OsI_11506 [Oryza sativa Indica Group] | | | | |
| | | | | &gt:gi\|222624903\|gb\|EEE59035.1\| hypothetical | | | | |
| | | | | protein OsJ_10788 [Oryza sativa Japonica Group] | | | | |
| | | NP_001060617 | 115474036 | Os07g0674700 [Oryza sativa Japonica Group] | 0.898936 | Oryza sativa Japonica Group | 2776 | 4196 |
| | | | | &gt:gi\|34393858\|dbj\|BAC83538.1\| putative | | | | |
| | | | | cytoplasmic ribosomal protein L18 | | | | |
| | | | | [Oryza sativa Japonica Group] | | | | |
| | | | | &gt:gi\|50509811\|dbj\|BAD31974.1\| | | | | |
| | | | | putative cytoplasmic ribosomal protein L18 | | | | |
| | | | | [Oryza sativa Japonica Group] | | | | |
| | | | | &gt:gi\|113612153\|dbj\|BAF22531.1\| Os07g0674700 | | | | |
| | | | | [Oryza sativa Japonica Group] | | | | |
| | | | | &gt:gi\|125559582\|gb\|EAZ05118.1\| hypothetical | | | | |
| | | | | protein OsI_27311 [Oryza sativa Indica Group] | | | | |
| | | | | &gt:gi\|215694724\|dbj\|BAG89915.1\| unnamed protein | | | | |
| | | | | product [Oryza sativa Japonica Group] | | | | |
| | | | | &gt:gi\|222637674\|gb\|EEE67806.1\| hypothetical | | | | |
| | | | | protein OsJ_25556 [Oryza sativa Japonica Group] | | | | |
| | 562-579 | XP_002445227 | 242080916 | hypothetical protein SORBIDRAFT_07g006330 | 1 | Sorghum bicolor | 2777 | 4197 |
| | | | | [Sorghum bicolor] | | | | |
| | | | | &gt:gi\|241941577\|gb\|EES14722.1\| | | | | |
| | | | | hypothetical protein SORBIDRAFT_07g006330 | | | | |
| | | | | [Sorghum bicolor] | | | | |
| | | NP_001168868 | 293333741 | hypothetical protein LOC100382673 [Zea mays] | 0.920875 | Zea mays | 2778 | 4198 |
| | | | | &gt:gi\|223973427\|gb\|ACN30901.1\| unknown | | | | |
| | | | | [Zea mays] | | | | |
| | | NP_001131176 | 212721253 | hypothetical protein LOC100192484 [Zea mays] | 0.914141 | Zea mays | 2779 | 4199 |
| | | | | &gt:gi\|194690786\|gb\|ACF79477.1\| unknown | | | | |
| | | | | [Zea mays] | | | | |
| | | EEC83121 | 54362548 | hypothetical protein OsI_28280 | 0.787879 | Oryza sativa Indica Group | 2780 | |
| | | | | [Oryza sativa Indica Group] | | | | |
| | | NP_001061275 | 115475356 | Os08g0224200 [Oryza sativa Japonica Group] | 0.786195 | Oryza sativa Japonica Group | 2781 | 4200 |
| | | | | &gt:gi\|38636667\|dbj\|BAD02987.1\| putative | | | | |
| | | | | Rubisco subunit binding-protein beta subunit | | | | |
| | | | | [Oryza sativa Japonica Group] | | | | |
| | | | | &gt:gi\|113623244\|dbj\|BAF23189.1\| | | | | |
| | | | | Os08g0224200 [Oryza sativa Japonica Group] | | | | |
| | | | | &gt:gi\|215715262\|dbj\|BAG95013.1\| unnamed | | | | |
| | | | | protein product [Oryza sativa Japonica Group] | | | | |
| | | EEE68253 | 54398660 | hypothetical protein OsJ_26460 | 0.740741 | Oryza sativa Japonica Group | 2782 | |
| | | | | [Oryza sativa Japonica Group] | | | | |
| | 544-561 | ACF83391 | 194698613 | unknown [Zea mays] | 1 | Zea mays | 2783 | 4201 |
| | | NP_001105647 | 162458977 | LOC542653 [Zea mays] | 0.993569 | Zea mays | 2784 | 4202 |
| | | | | &gt:gi\|28950670\|gb\|AAO63267.1\| | | | | |
| | | | | legumin-like protein [Zea mays] | | | | |
| | | ACG36012 | 195628363 | legumin-like protein [Zea mays] | 0.987138 | Zea mays | 2785 | 4203 |
| | | XP_002440480 | 242089294 | hypothetical protein SORBIDRAFT_09g001680 | 0.874598 | Sorghum bicolor | 2786 | 4204 |
| | | | | [Sorghum bicolor] | | | | |
| | | | | &gt:gi\|241945765\|gb\|EES18910.1\| | | | | |
| | | | | hypothetical protein SORBIDRAFT_09g001680 | | | | |
| | | | | [Sorghum bicolor] | | | | |
| | | NP_001150066 | 226498887 | legumin-like protein [Zea mays] | 0.881029 | Zea mays | 2787 | 4205 |
| | | | | &gt:gi\|195607700\|gb\|ACG25680.1\| | | | | |
| | | | | legumin-like protein [Zea mays] | | | | |
| | | | | &gt:gi\|195636432\|gb\|ACG37684.1\| | | | | |
| | | | | legumin-like protein [Zea mays] | | | | |
| | | | | &gt:gi\|224033321\|gb\|ACN35736.1\| | | | | |
| | | | | unknown [Zea mays] | | | | |
| | | NP_001105062 | 162459029 | legumin-like protein [Zea mays] | 0.893891 | Zea mays | 2788 | 4206 |
| | | | | &gt:gi\|28950668\|gb\|AAO63266.1\| | | | | |
| | | | | legumin-like protein [Zea mays] | | | | |
| | | ACG36544 | 195629805 | legumin-like protein [Zea mays] | 0.890675 | Zea mays | 2789 | 4207 |
| | | BAJ94492 | 326494745 | predicted protein | 0.85209 | Hordeum vulgare subsp. vulgare | 2790 | 4208 |
| | | | | [Hordeum vulgare subsp. vulgare] | | | | |
| | | | | &gt:gi\|326511543\|dbj\|BAJ91916.1\| predicted | | | | |
| | | | | protein [Hordeum vulgare subsp. vulgare] | | | | |
| | | NP_001054469 | 115461738 | Os05g0116000 [Oryza sativa Japonica Group] | 0.784566 | Oryza sativa Japonica Group | 2791 | 4209 |
| | | | | &gt:gi\|52353520\|gb\|AAU44086.1\| putative | | | | |
| | | | | legumin [Oryza sativa Japonica Group] | | | | |
| | | | | &gt:gi\|55168333\|gb\|AAV44198.1\| putative | | | | |
| | | | | legumin [Oryza sativa Japonica Group] | | | | |
| | | | | &gt:gi\|113578020\|dbj\|BAF16383.1\| Os05g0116000 | | | | |
| | | | | [Oryza sativa Japonica Group] | | | | |
| | | | | &gt:gi\|125550596\|gb\|EAY96305.1\| hypothetical | | | | |
| | | | | protein OsI_18204 [Oryza sativa Indica Group] | | | | |
| | | | | &gt:gi\|215764963\|dbj\|BAG86660.1\| unnamed | | | | |
| | | | | protein product [Oryza sativa Japonica Group] | | | | |
| | | | | &gt:gi\|215768422\|dbj\|BAH00651.1\| unnamed | | | | |
| | | | | protein product [Oryza sativa Japonica Group] | | | | |
| | | EEE62108 | 54398660 | hypothetical protein OsJ_16892 | 0.78135 | Oryza sativa Japonica Group | 2792 | |
| | | | | [Oryza sativa Japonica Group] | | | | |
| | 849-866 | XP_002458930 | 242059568 | hypothetical protein SORBIDRAFT_03g042880 | 1 | Sorghum bicolor | 2793 | 4210 |
| | | | | [Sorghum bicolor] | | | | |
| | | | | &gt:gi\|241930905\|gb\|EES04050.1\| | | | | |
| | | | | hypothetical protein SORBIDRAFT_03g042880 | | | | |
| | | | | [Sorghum bicolor] | | | | |
| | | NP_001169575 | 293331910 | hypothetical protein LOC100383455 [Zea mays] | 0.900249 | Zea mays | 2794 | 4211 |
| | | | | &gt:gi\|224030179\|gb\|ACN34165.1\| unknown | | | | |
| | | | | [Zea mays] | | | | |
| | | EEC71972 | 54362548 | hypothetical protein OsI_04809 | 0.869077 | Oryza sativa Indica Group | 2795 | |
| | | | | [Oryza sativa Indica Group] | | | | |
| | | NP_001045108 | 115441656 | Os01g0901000 [Oryza sativa Japonica Group] | 0.86409 | Oryza sativa Japonica Group | 2796 | 4212 |
| | | | | &gt:gi\|56784865\|dbj\|BAD82105.1\| putative | | | | |
| | | | | bg55 [Oryza sativa Japonica Group] | | | | |
| | | | | &gt:gi\|113534639\|dbj\|BAF07022.1\| Os01g0901000 | | | | |
| | | | | [Oryza sativa Japonica Group] | | | | |
| | | | | &gt:gi\|215715204\|dbj\|BAG94955.1\| unnamed | | | | |
| | | | | protein product [Oryza sativa Japonica Group] | | | | |
| | | BAB90523 | 20161598 | B1065G12.5 [Oryza sativa Japonica Group] | 0.814214 | Oryza sativa Japonica Group | 2797 | 4213 |
| | | BAK03281 | 326512747 | predicted protein | 0.837905 | Hordeum vulgare subsp. vulgare | 2798 | 4214 |
| | | | | [Hordeum vulgare subsp. vulgare] | | | | |
| | | EEE55827 | 54398660 | hypothetical protein OsJ_04433 | 0.820449 | Oryza sativa Japonica Group | 2799 | |
| | | | | [Oryza sativa Japonica Group] | | | | |
| | 1153-1170 | NP_001151583 | 226492901 | WRKY74 - superfamily of TFs having WRKY and | 1 | Zea mays | 2800 | 4215 |
| | | | | zinc finger domains [Zea mays] | | | | |
| | | | | &gt:gi\|195647918\|gb\|ACG43427.1\| WRKY74 - | | | | |
| | | | | superfamily of TFs having WRKY and zinc | | | | |
| | | | | finger domains [Zea mays] | | | | |
| | | XP_002460068 | 242044393 | hypothetical protein SORBIDRAFT_02g022280 | 0.774834 | Sorghum bicolor | 2801 | 4216 |
| | | | | [Sorghum bicolor] | | | | |
| | | | | &gt:gi\|241923445\|gb\|EER96589.1\| | | | | |
| | | | | hypothetical protein SORBIDRAFT_02g022280 | | | | |
| | | | | [Sorghum bicolor] | | | | |
| Predicted | 822-839 | XP_002455452 | 242052612 | hypothetical protein SORBIDRAFT_03g011010 | 1 | Sorghum bicolor | 2802 | 4217 |
| siRNA | | | | [Sorghum bicolor] | | | | |
| 58108 | | | | &gt:gi\|63087720\|emb\|CAI93175.1\| | | | | |
| | | | | glycosyltransferase [Sorghum bicolor] | | | | |
| | | | | &gt:gi\|241927427\|gb\|EES00572.1\| hypothetical | | | | |
| | | | | protein SORBIDRAFT_03g011010 | | | | |
| | | | | [Sorghum bicolor] | | | | |
| | | NP_001105783 | 162459421 | glycosyltransferase [Zea mays] | 0.876471 | Zea mays | 2803 | 4218 |
| | | | | &gt:gi\|63087722\|emb\|CAI93176.1\| | | | | |
| | | | | glycosyltransferase [Zea mays] | | | | |
| | 285-302 | NP_001132904 | 212723947 | hypothetical protein LOC100194403 [Zea mays] | 1 | Zea mays | 2804 | 4219 |
| | | | | &gt:gi\|194695716\|gb\|ACF81942.1\| unknown | | | | |
| | | | | [Zea mays] | | | | |
| | | AEF30550 | 333385002 | serine/threonine protein kinase Stpk-B | 0.77037 | Triticum aestivum | 2805 | 4220 |
| | | | | [Triticum aestivum] | | | | |
| | | AEF30549 | 333385000 | serine/threonine protein kinase Stpk-D | 0.762963 | Triticum aestivum | 2806 | 4221 |
| | | | | [Triticum aestivum] | | | | |
| | | BAJ93188 | 326526022 | predicted protein | 0.762963 | Hordeum vulgare subsp. vulgare | 2807 | 4222 |
| | | | | [Hordeum vulgare subsp. vulgare] | | | | |
| | | AEF30546 | 333384994 | serine/threonine protein kinase Stpk-V | 0.760494 | Dasypyrum villosum | 2808 | 4223 |
| | | | | [Dasypyrum villosum] | | | | |
| | | | | &gt:gi\|333384997\|gb\|AEF30547.1\| | | | | |
| | | | | serine/threonine protein kinase Stpk-V | | | | |
| | | | | [Dasypyrum villosum] | | | | |
| | | AEF30548 | 333384998 | serine/threonine protein kinase Stpk-A | 0.760494 | Triticum aestivum | 2809 | 4224 |
| | | | | [Triticum aestivum] | | | | |
| | | NP_001045995 | 115444430 | Os02g0165100 [Oryza sativa Japonica Group] | 0.745679 | Oryza sativa Japonica Group | 2810 | 4225 |
| | | | | &gt:gi\|49388058\|dbj\|BAD25172.1\| putative receptor | | | | |
| | | | | protein kinase PERK [Oryza sativa Japonica Group] | | | | |
| | | | | &gt:gi\|49388415\|dbj\|BAD25548.1\| putative receptor | | | | |
| | | | | protein kinase PERK [Oryza sativa Japonica Group] | | | | |
| | | | | &gt:gi\|113535526\|dbj\|BAF07909.1\| Os02g0165100 | | | | |
| | | | | [Oryza sativa Japonica Group] | | | | |
| | | | | &gt:gi\|215694876\|dbj\|BAG90067.1\| unnamed | | | | |
| | | | | protein product [Oryza sativa Japonica Group] | | | | |
| | | EEE56371 | 54398660 | hypothetical protein OsJ_05508 | 0.74321 | Oryza sativa Japonica Group | 2811 | |
| | | | | [Oryza sativa Japonica Group] | | | | |
| | 677-694 | EEE56138 | 54398660 | hypothetical protein OsJ_05018 | 1 | Oryza sativa Japonica Group | 2812 | |
| | | | | [Oryza sativa Japonica Group] | | | | |
| | | NP_001045614 | 115443668 | Os02g0104700 [Oryza sativa Japonica Group] | 0.98829 | Oryza sativa Japonica Group | 2813 | 4226 |
| | | | | &gt:gi\|75131025\|sp\|Q6YPG5.1\|NOS_ORYSJ | | | | |
| | | | | RecName: Full = Putative nitric oxide synthase | | | | |
| | | | | &gt:gi\|40363768\|dbj\|BAD06278.1\| putative | | | | |
| | | | | GTPase [Oryza sativa Japonica Group] | | | | |
| | | | | &gt:gi\|41052546\|dbj\|BAD07538.1\| putative | | | | |
| | | | | GTPase [Oryza sativa Japonica Group] | | | | |
| | | | | &gt:gi\|113535145\|dbj\|BAF07528.1\| Os02g0104700 | | | | |
| | | | | [Oryza sativa Japonica Group] | | | | |
| | | EAY84101 | 54362548 | hypothetical protein OsI_05484 | 0.985948 | Oryza sativa Indica Group | 2814 | |
| | | | | [Oryza sativa Indica Group] | | | | |
| | | NP_001168044 | 293331104 | hypothetical protein LOC100381773 [Zea mays] | 0.894614 | Zea mays | 2815 | 4227 |
| | | | | &gt:gi\|223945667\|gb\|ACN26917.1\| unknown | | | | |
| | | | | [Zea mays] | | | | |
| | | ADK47527 | 301015572 | NOA1/RIF1 protein [Medicago truncatula] | 0.730679 | Medicago truncatula | 2816 | 4228 |
| | 640-657 | XP_002446751 | 242073629 | hypothetical protein SORBIDRAFT_06g021760 | 1 | Sorghum bicolor | 2817 | 4229 |
| | | | | [Sorghum bicolor] | | | | |
| | | | | &gt:gi\|241937934\|gb\|EES11079.1\| | | | | |
| | | | | hypothetical protein SORBIDRAFT_06g021760 | | | | |
| | | | | [Sorghum bicolor] | | | | |
| | | NP_001053236 | 115459271 | Os04g0502800 [Oryza sativa Japonica Group] | 0.883362 | Oryza sativa Japonica Group | 2818 | 4230 |
| | | | | &gt:gi\|113564807\|dbj\|BAF15150.1\| Os04g0502800 | | | | |
| | | | | [Oryza sativa Japonica Group] | | | | |
| | | BAK00120 | 326519493 | predicted protein | 0.866209 | Hordeum vulgare subsp. vulgare | 2819 | 4231 |
| | | | | [Hordeum vulgare subsp. vulgare] | | | | |
| | | EEE61279 | 54398660 | hypothetical protein OsJ_15362 | 0.881647 | Oryza sativa Japonica Group | 2820 | |
| | | | | [Oryza sativa Japonica Group] | | | | |
| | | CAE02931 | 70663914 | OSJNBa0014K14.3 | 0.881647 | Oryza sativa Japonica Group | 2821 | 4232 |
| | | | | [Oryza sativa Japonica Group] | | | | |
| | | CAH67585 | 116310792 | OSIGBa0112M24.2 | 0.883362 | Oryza sativa Indica Group | 2822 | 4233 |
| | | | | [Oryza sativa Indica Group] | | | | |
| | | EEC77582 | 54362548 | hypothetical protein OsI_16534 | 0.883362 | Oryza sativa Indica Group | 2823 | |
| | | | | [Oryza sativa Indica Group] | | | | |
| | | NP_001168646 | 293332436 | hypothetical protein LOC100382433 [Zea mays] | 0.70669 | Zea mays | 2824 | 4234 |
| | | | | &gt:gi\|223949891\|gb\|ACN29029.1\| unknown | | | | |
| | | | | [Zea mays] | | | | |
| | 927-944 | XP_002451348 | 242060117 | hypothetical protein SORBIDRAFT_04g000530 | 1 | Sorghum bicolor | 2825 | 4235 |
| | | | | [Sorghum bicolor] | | | | |
| | | | | &gt:gi\|241931179\|gb\|EES04324.1\| | | | | |
| | | | | hypothetical protein SORBIDRAFT_04g000530 | | | | |
| | | | | [Sorghum bicolor] | | | | |
| | | BAK03882 | 326519914 | predicted protein | 0.838828 | Hordeum vulgare subsp. vulgare | 2826 | 4236 |
| | | | | [Hordeum vulgare subsp. vulgare] | | | | |
| | 1528-1545 | NP_001143089 | 226498753 | hypothetical protein LOC100275564 [Zea mays] | 1 | Zea mays | 2827 | 4237 |
| | | | | &gt:gi\|195614134\|gb\|ACG28897.1\| hypothetical | | | | |
| | | | | protein [Zea mays] | | | | |
| | | XP_002453857 | 242065135 | hypothetical protein SORBIDRAFT_04g019720 | 0.866758 | Sorghum bicolor | 2828 | 4238 |
| | | | | [Sorghum bicolor] | | | | |
| | | | | &gt:gi\|241933688\|gb\|EES06833.1\| | | | | |
| | | | | hypothetical protein SORBIDRAFT_04g019720 | | | | |
| | | | | [Sorghum bicolor] | | | | |
| | | EAY74088 | 54362548 | hypothetical protein OsI_01975 | 0.739011 | Oryza sativa Indica Group | 2829 | |
| | | | | [Oryza sativa Indica Group] | | | | |
| | | BAB61143 | 14587202 | unknown protein [Oryza sativa Japonica Group] | 0.737637 | Oryza sativa Japonica Group | 2830 | 4239 |
| | | | | &gt:gi\|15408639\|dbj\|BAB64056.1\| unknown protein | | | | |
| | | | | [Oryza sativa Japonica Group] | | | | |
| | | | | &gt:gi\|125570433\|gb\|EAZ11948.1\| hypothetical | | | | |
| | | | | protein OsJ_01821 [Oryza sativa Japonica Group] | | | | |
| | | | | &gt:gi\|215741489\|dbj\|BAG97984.1\| unnamed protein | | | | |
| | | | | product [Oryza sativa Japonica Group] | | | | |
| Predicted | 1481-1501 | ACL53547 | 219886344 | unknown [Zea mays] | 1 | Zea mays | 2831 | 4240 |
| siRNA | | | | | | | | |
| 58158 | | | | | | | | |
| | | ACL54434 | 219888118 | unknown [Zea mays] | 0.964789 | Zea mays | 2832 | 4241 |
| | | ACF88046 | 194707923 | unknown [Zea mays] | 0.964789 | Zea mays | 2833 | 4242 |
| | | ACG44902 | 195650868 | ureide permease 2 [Zea mays] | 0.960094 | Zea mays | 2834 | 4243 |
| | | NP_001066832 | 115488689 | Os12g0502800 [Oryza sativa Japonica Group] | 0.852113 | Oryza sativa Japonica Group | 2835 | 4244 |
| | | | | &gt:gi\|77555848\|gb\|ABA98644.1\| Ureide permease 2, | | | | |
| | | | | putative, expressed [Oryza sativa Japonica Group] | | | | |
| | | | | &gt:gi\|113649339\|dbj\|BAF29851.1\| Os12g0502800 | | | | |
| | | | | [Oryza sativa Japonica Group] | | | | |
| | | | | &gt:gi\|215707179\|dbj\|BAG93639.1\| unnamed protein | | | | |
| | | | | product [Oryza sativa Japonica Group] | | | | |
| | | | | &gt:gi\|215741152\|dbj\|BAG97647.1\| unnamed protein | | | | |
| | | | | product [Oryza sativa Japonica Group] | | | | |
| | | ABA98643 | 108862058 | Ureide permease 2, putative, expressed | 0.852113 | Oryza sativa Japonica Group | 2836 | |
| | | | | [Oryza sativa Japonica Group] | | | | |
| | | | | &gt:gi\|218186904\|gb\|EEC69331.1\| | | | | |
| | | | | hypothetical protein OsI_38433 | | | | |
| | | | | [Oryza sativa Indica Group] | | | | |
| | | | | &gt:gi\|222617132\|gb\|EEE53264.1\| | | | | |
| | | | | hypothetical protein OsJ_36199 | | | | |
| | | | | [Oryza sativa Japonica Group] | | | | |
| | | BAK08299 | 326530038 | predicted protein | 0.826291 | Hordeum vulgare subsp. vulgare | 2837 | 4245 |
| | | | | [Hordeum vulgare subsp. vulgare] | | | | |
| | | NP_001183878 | 308081201 | hypothetical protein LOC100502471 [Zea mays] | 0.873239 | Zea mays | 2838 | 4246 |
| | | | | &gt:gi\|238015200\|gb\|ACR38635.1\| unknown | | | | |
| | | | | [Zea mays] | | | | |
| | | ACF22675 | 193848478 | ureide permease 2-like protein 2 | 0.701878 | Brachypodium distachyon | 2839 | |
| | | | | [Brachypodium distachyon] | | | | |
| | 113-133 | XP_002463060 | 242050631 | hypothetical protein SORBIDRAFT_02g036920 | 1 | Sorghum bicolor | 2840 | 4247 |
| | | | | [Sorghum bicolor] | | | | |
| | | | | &gt:gi\|241926437\|gb\|EER99581.1\| | | | | |
| | | | | hypothetical protein SORBIDRAFT_02g036920 | | | | |
| | | | | [Sorghum bicolor] | | | | |
| | | NP_001168223 | 293336038 | hypothetical protein LOC100381982 [Zea mays] | 0.94206 | Zea mays | 2841 | 4248 |
| | | | | &gt:gi\|223946819\|gb\|ACN27493.1\| unknown | | | | |
| | | | | [Zea mays] | | | | |
| | | EEC82301 | 54362548 | hypothetical protein OsI_26552 | 0.76824 | Oryza sativa Indica Group | 2842 | |
| | | | | [Oryza sativa Indica Group] | | | | |
| | | NP_001060053 | 115472908 | Os07g0571700 [Oryza sativa Japonica Group] | 0.76824 | Oryza sativa Japonica Group | 2843 | 4249 |
| | | | | &gt:gi\|34394216\|dbj\|BAC84668.1\| putative organic | | | | |
| | | | | cation transporter [Oryza sativa Japonica Group] | | | | |
| | | | | &gt:gi\|113611589\|dbj\|BAF21967.1\| Os07g0571700 | | | | |
| | | | | [Oryza sativa Japonica Group] | | | | |
| | 1396-1416 | XP_002460562 | 242045381 | hypothetical protein SORBIDRAFT_02g030730 | 1 | Sorghum bicolor | 2844 | 4250 |
| | | | | [Sorghum bicolor] | | | | |
| | | | | &gt:gi\|241923939\|gb\|EER97083.1\| | | | | |
| | | | | hypothetical protein SORBIDRAFT_02g030730 | | | | |
| | | | | [Sorghum bicolor] | | | | |
| | | XP_002460563 | 242045383 | hypothetical protein SORBIDRAFT_02g030740 | 0.893069 | Sorghum bicolor | 2845 | 4251 |
| | | | | [Sorghum bicolor] | | | | |
| | | | | &gt:gi\|241923940\|gb\|EER97084.1\| | | | | |
| | | | | hypothetical protein SORBIDRAFT_02g030740 | | | | |
| | | | | [Sorghum bicolor] | | | | |
| | | NP_001168434 | 293334700 | hypothetical protein LOC100382204 [Zea mays] | 0.881188 | Zea mays | 2846 | 4252 |
| | | | | &gt:gi\|223948277\|gb\|ACN28222.1\| unknown | | | | |
| | | | | [Zea mays] | | | | |
| | | XP_002460167 | 242044591 | hypothetical protein SORBIDRAFT_02g023770 | 0.875248 | Sorghum bicolor | 2847 | 4253 |
| | | | | [Sorghum bicolor] | | | | |
| | | | | &gt:gi\|241923544\|gb\|EER96688.1\| | | | | |
| | | | | hypothetical protein SORBIDRAFT_02g023770 | | | | |
| | | | | [Sorghum bicolor] | | | | |
| | | XP_002460561 | 242045379 | hypothetical protein SORBIDRAFT_02g030720 | 0.786139 | Sorghum bicolor | 2848 | 4254 |
| | | | | [Sorghum bicolor] | | | | |
| | | | | &gt:gi\|241923938\|gb\|EER97082.1\| | | | | |
| | | | | hypothetical protein SORBIDRAFT_02g030720 | | | | |
| | | | | [Sorghum bicolor] | | | | |
| | 1610-1630 | ACN31936 | 223975496 | unknown [Zea mays] | 1 | Zea mays | 2849 | 4255 |
| | 1382-1402 | NP_001183878 | 308081201 | hypothetical protein LOC100502471 [Zea mays] | 1 | Zea mays | 2850 | 4256 |
| | | | | &gt:gi\|238015200\|gb\|ACR38635.1\| unknown | | | | |
| | | | | [Zea mays] | | | | |
| | 479-499 | XP_002460117 | 242044491 | hypothetical protein SORBIDRAFT_02g023000 | 1 | Sorghum bicolor | 2851 | 4257 |
| | | | | [Sorghum bicolor] | | | | |
| | | | | &gt:gi\|241923494\|gb\|EER96638.1\| | | | | |
| | | | | hypothetical protein SORBIDRAFT_02g023000 | | | | |
| | | | | [Sorghum bicolor] | | | | |
| | | ACR37870 | 238013669 | unknown [Zea mays] | 0.946809 | Zea mays | 2852 | 4258 |
| | | NP_001143802 | 226505329 | hypothetical protein LOC100276574 [Zea mays] | 0.93883 | Zea mays | 2853 | 4259 |
| | | | | &gt:gi\|195627382\|gb\|ACG35521.1\| hypothetical | | | | |
| | | | | protein [Zea mays] | | | | |
| | | BAD23415 | 48716714 | hydrolase-like protein [Oryza sativa Japonica Group] | 0.702128 | Oryza sativa Japonica Group | 2854 | 4260 |
| | | | | &gt:gi\|50726197\|dbj\|BAD33716.1\| hydrolase-like | | | | |
| | | | | protein [Oryza sativa Japonica Group] | | | | |
| | | BAJ92435 | 326516559 | predicted protein | 0.744681 | Hordeum vulgare subsp. vulgare | 2855 | 4261 |
| | | | | [Hordeum vulgare subsp. vulgare] | | | | |
| | | EEC84446 | 54362548 | hypothetical protein OsI_31065 | 0.742021 | Oryza sativa Indica Group | 2856 | |
| | | | | [Oryza sativa Indica Group] | | | | |
| | | | | &gt:gi\|222641433\|gb\|EEE69565.1\| | | | | |
| | | | | hypothetical protein OsJ_29077 | | | | |
| | | | | [Oryza sativa Japonica Group] | | | | |
| | 577-597 | XP_002456713 | 242055134 | hypothetical protein SORBIDRAFT_03g041220 | 1 | Sorghum bicolor | 2857 | 4262 |
| | | | | [Sorghum bicolor] | | | | |
| | | | | &gt:gi\|241928688\|gb\|EES01833.1\| | | | | |
| | | | | hypothetical protein SORBIDRAFT_03g041220 | | | | |
| | | | | [Sorghum bicolor] | | | | |
| | | NP_001147599 | 226506121 | POT family protein [Zea mays] | 0.790353 | Zea mays | 2858 | 4263 |
| | | | | &gt:gi\|195612430\|gb\|ACG28045.1\| POT | | | | |
| | | | | family protein [Zea mays] | | | | |
| Predicted | 1081-1100 | ACR37939 | 238013807 | unknown [Zea mays] | 1 | Zea mays | 2859 | 4264 |
| siRNA | | | | | | | | |
| 58740 | | | | | | | | |
| | | NP_001150768 | 226495128 | LOC100284401 [Zea mays] | 0.996764 | Zea mays | 2860 | 4265 |
| | | | | &gt:gi\|195641698\|gb\|ACG40317.1\| | | | | |
| | | | | ubiquitinating enzyme [Zea mays] | | | | |
| | | NP_001049901 | 115452600 | Os03g0308000 [Oryza sativa Japonica Group] | 0.76699 | Oryza sativa Japonica Group | 2861 | 4266 |
| | | | | &gt:gi\|108707750\|gb\|ABF95545.1\| | | | | |
| | | | | ubiquitinating enzyme, putative, expressed | | | | |
| | | | | [Oryza sativa Japonica Group] | | | | |
| | | | | &gt:gi\|113548372\|dbj\|BAF11815.1\| | | | | |
| | | | | Os03g0308000 [Oryza sativa Japonica Group] | | | | |
| | | | | &gt:gi\|215713428\|dbj\|BAG94565.1\| unnamed | | | | |
| | | | | protein product [Oryza sativa Japonica Group] | | | | |
| | | EEC75094 | 54362548 | hypothetical protein OsI_11252 | 0.760518 | Oryza sativa Indica Group | 2862 | |
| | | | | [Oryza sativa Indica Group] | | | | |
| | | BAJ86904 | 326509022 | predicted protein | 0.760518 | Hordeum vulgare subsp. vulgare | 2863 | 4267 |
| | | | | [Hordeum vulgare subsp. vulgare] | | | | |
| | | EEE58922 | 54398660 | hypothetical protein OsJ_10572 | 0.737864 | Oryza sativa Japonica Group | 2864 | |
| | | | | [Oryza sativa Japonica Group] | | | | |
| Predicted | 410-428 | NP_001169291 | 293331114 | hypothetical protein LOC100383155 [Zea mays] | 1 | Zea mays | 2865 | 4268 |
| siRNA | | | | &gt:gi\|224028455\|gb\|ACN33303.1\| unknown | | | | |
| 59056 | | | | [Zea mays] | | | | |
| | 211-229 | NP_001052652 | 115458103 | Os04g0394200 [Oryza sativa Japonica Group] | 1 | Oryza sativa Japonica Group | 2866 | 4269 |
| | | | | &gt:gi\|21740743\|emb\|CAD40552.1\| | | | | |
| | | | | OSJNBa0072K14.5 [Oryza sativa Japonica Group] | | | | |
| | | | | &gt:gi\|113564223\|dbj\|BAF14566.1\| Os04g0394200 | | | | |
| | | | | [Oryza sativa Japonica Group] | | | | |
| | | | | &gt:gi\|116309381\|emb\|CAH66460.1\| H0718E12.4 | | | | |
| | | | | [Oryza sativa Indica Group] | | | | |
| | | | | &gt:gi\|125590215\|gb\|EAZ30565.1\| hypothetical | | | | |
| | | | | protein OsJ_14615 [Oryza sativa Japonica Group] | | | | |
| | | | | &gt:gi\|215768038\|dbj\|BAH00267.1\| unnamed | | | | |
| | | | | protein product [Oryza sativa Japonica Group] | | | | |
| | | ACG35848 | 195628035 | dihydrolipoyllysine-residue succinyltransferase | 0.884091 | Zea mays | 2867 | 4270 |
| | | | | component of 2-oxoglutarate dehydrogenase complex | | | | |
| | | | | [Zea mays] | | | | |
| | | NP_001147014 | 226509379 | dihydrolipoyllysine-residue succinyltransferase | 0.881818 | Zea mays | 2868 | 4271 |
| | | | | component of 2-oxoglutarate dehydrogenase complex | | | | |
| | | | | [Zea mays] | | | | |
| | | | | &gt:gi\|195606476\|gb\|ACG25068.1\| | | | | |
| | | | | dihydrolipoyllysine-residue succinyltransferase | | | | |
| | | | | component of 2-oxoglutarate dehydrogenase complex | | | | |
| | | | | [Zea mays] | | | | |
| | | ACR38672 | 238015273 | unknown [Zea mays] | 0.827273 | Zea mays | 2869 | 4272 |
| | | BAJ96018 | 326512073 | predicted protein | 0.813636 | Hordeum vulgare subsp. vulgare | 2870 | 4273 |
| | | | | [Hordeum vulgare subsp. vulgare] | | | | |
| | | BAD22992 | 48716367 | putative 2-oxoglutarate dehydrogenase E2 | 0.734091 | Oryza sativa Japonica Group | 2871 | 4274 |
| | | | | subunit [Oryza sativa Japonica Group] | | | | |
| | 28-Oct | NP_001045255 | 115441950 | Os01g0925300 [Oryza sativa Japonica Group] | 1 | Oryza sativa Japonica Group | 2872 | 4275 |
| | | | | &gt:gi\|57899394\|dbj\|BAD88041.1\| putative | | | | |
| | | | | zisp [Oryza sativa Japonica Group] | | | | |
| | | | | &gt:gi\|57900122\|dbj\|BAD88184.1\| putative | | | | |
| | | | | zisp [Oryza sativa Japonica Group] | | | | |
| | | | | &gt:gi\|113534786\|dbj\|BAF07169.1\| Os01g0925300 | | | | |
| | | | | [Oryza sativa Japonica Group] | | | | |
| | | EAZ14683 | 54398660 | hypothetical protein OsJ_04607 | 0.961259 | Oryza sativa Japonica Group | 2873 | |
| | | | | [Oryza sativa Japonica Group] | | | | |
| | | BAB89658 | 20160711 | P0482D04.5 [Oryza sativa Japonica Group] | 0.949153 | Oryza sativa Japonica Group | 2874 | 4276 |
| | | EEC72069 | 54362548 | hypothetical protein OsI_05001 | 0.932203 | Oryza sativa Indica Group | 2875 | |
| | | | | [Oryza sativa Indica Group] | | | | |
| | | NP_001130802 | 212274974 | hypothetical protein LOC100191906 [Zea mays] | 0.849879 | Zea mays | 2876 | 4277 |
| | | | | &gt:gi\|194690056\|gb\|ACF79112.1\| unknown | | | | |
| | | | | [Zea mays] | | | | |
| | | | | &gt:gi\|194690150\|gb\|ACF79159.1\| | | | | |
| | | | | unknown [Zea mays] | | | | |
| | | | | &gt:gi\|195635627\|gb\|ACG37282.1\| | | | | |
| | | | | palmitoyltransferase ZDHHC9 [Zea mays] | | | | |
| | | | | &gt:gi\|224030505\|gb\|ACN34328.1\| unknown | | | | |
| | | | | [Zea mays] | | | | |
| | | BAK03564 | 326515301 | predicted protein | 0.8523 | Hordeum vulgare subsp. vulgare | 2877 | 4278 |
| | | | | [Hordeum vulgare subsp. vulgare] | | | | |
| | | XP_002459011 | 242059730 | hypothetical protein SORBIDRAFT_03g044420 | 0.769976 | Sorghum bicolor | 2878 | 4279 |
| | | | | [Sorghum bicolor] | | | | |
| | | | | &gt:gi\|241930986\|gb\|EES04131.1\| | | | | |
| | | | | hypothetical protein SORBIDRAFT_03g044420 | | | | |
| | | | | [Sorghum bicolor] | | | | |
| | 137-155 | NP_001056918 | 115466637 | Os06g0167100 [Oryza sativa Japonica Group] | 1 | Oryza sativa Japonica Group | 2879 | 4280 |
| | | | | &gt:gi\|55296045\|dbj\|BAD67607.1\| putative | | | | |
| | | | | gamma-adaptin 1 [Oryza sativa Japonica Group] | | | | |
| | | | | &gt:gi\|113594958\|dbj\|BAF18832.1\| Os06g0167100 | | | | |
| | | | | [Oryza sativa Japonica Group] | | | | |
| | | | | &gt:gi\|215678749\|dbj\|BAG95186.1\| unnamed protein | | | | |
| | | | | product [Oryza sativa Japonica Group] | | | | |
| | | | | &gt:gi\|222635027\|gb\|EEE65159.1\| hypothetical | | | | |
| | | | | protein OsJ_20260 [Oryza sativa Japonica Group] | | | | |
| | | EEC80086 | 54362548 | hypothetical protein OsI_21821 | 0.95977 | Oryza sativa Indica Group | 2880 | |
| | | | | [Oryza sativa Indica Group] | | | | |
| | | BAK00520 | 326524272 | predicted protein | 0.889655 | Hordeum vulgare subsp. vulgare | 2881 | 4281 |
| | | | | [Hordeum vulgare subsp. vulgare] | | | | |
| | | ACG28758 | 195613855 | AP-1 complex subunit gamma-1 [Zea mays] | 0.894253 | Zea mays | 2882 | 4282 |
| | | XP_002454734 | 242066889 | hypothetical protein SORBIDRAFT_04g036416 | 0.821839 | Sorghum bicolor | 2883 | 4283 |
| | | | | [Sorghum bicolor] | | | | |
| | | | | &gt:gi\|241934565\|gb\|EES07710.1\| | | | | |
| | | | | hypothetical protein SORBIDRAFT_04g036416 | | | | |
| | | | | [Sorghum bicolor] | | | | |
| | | EEC74194 | 54362548 | hypothetical protein OsI_09342 | 0.825287 | Oryza sativa Indica Group | 2884 | |
| | | | | [Oryza sativa Indica Group] | | | | |
| | | XP_002521026 | 255560014 | AP-1 complex subunit gamma-2, putative | 0.733333 | Ricinus communis | 2885 | 4284 |
| | | | | [Ricinus communis] | | | | |
| | | | | &gt:gi\|223539863\|gb\|EEF41443.1\| | | | | |
| | | | | AP-1 complex subunit gamma-2, putative | | | | |
| | | | | [Ricinus communis] | | | | |
| | | XP_002265190 | 225424712 | PREDICTED: hypothetical protein [Vitis vinifera] | 0.732184 | Vitis vinifera | 2886 | 4285 |
| | | | | &gt:gi\|296086533\|emb\|CBI32122.3\| unnamed protein | | | | |
| | | | | product [Vitis vinifera] | | | | |
| | 431-449 | XP_002456313 | 242054334 | hypothetical protein SORBIDRAFT_03g033840 | 1 | Sorghum bicolor | 2887 | 4286 |
| | | | | [Sorghum bicolor] | | | | |
| | | | | &gt:gi\|241928288\|gb\|EES01433.1\| | | | | |
| | | | | hypothetical protein SORBIDRAFT_03g033840 | | | | |
| | | | | [Sorghum bicolor] | | | | |
| | | NP_001168082 | 293331612 | hypothetical protein LOC100381816 [Zea mays] | 0.89852 | Zea mays | 2888 | 4287 |
| | | | | &gt:gi\|223945895\|gb\|ACN27031.1\| unknown | | | | |
| | | | | [Zea mays] | | | | |
| | | XP_002458462 | 242058632 | hypothetical protein SORBIDRAFT_03g034130 | 0.701903 | Sorghum bicolor | 2889 | 4288 |
| | | | | [Sorghum bicolor] | | | | |
| | | | | &gt:gi\|241930437\|gb\|EES03582.1\| | | | | |
| | | | | hypothetical protein SORBIDRAFT_03g034130 | | | | |
| | | | | [Sorghum bicolor] | | | | |
| | 84-102 | ACN30664 | 223972952 | unknown [Zea mays] | 1 | Zea mays | 2890 | 4289 |
| | 626-644 | NP_001150636 | 226532023 | dihydrolipoyllysine-residue succinyltransferase | 1 | Zea mays | 2891 | 4290 |
| | | | | component of 2-oxoglutarate dehydrogenase complex | | | | |
| | | | | [Zea mays] | | | | |
| | | | | &gt:gi\|195640766\|gb\|ACG39851.1\| | | | | |
| | | | | dihydrolipoyllysine-residue succinyltransferase | | | | |
| | | | | component of 2-oxoglutarate dehydrogenase | | | | |
| | | | | complex [Zea mays] | | | | |
| | 235-253 | XP_002447329 | 242074785 | hypothetical protein SORBIDRAFT_06g033040 | 1 | Sorghum bicolor | 2892 | 4291 |
| | | | | [Sorghum bicolor] | | | | |
| | | | | &gt:gi\|241938512\|gb\|EES11657.1\| | | | | |
| | | | | hypothetical protein SORBIDRAFT_06g033040 | | | | |
| | | | | [Sorghum bicolor] | | | | |
| | | ACN26581 | 223944994 | unknown [Zea mays] | 0.912548 | Zea mays | 2893 | 4292 |
| | | XP_002448044 | 242076215 | hypothetical protein SORBIDRAFT_06g020130 | 0.901141 | Sorghum bicolor | 2894 | 4293 |
| | | | | [Sorghum bicolor] | | | | |
| | | | | &gt:gi\|241939227\|gb\|EES12372.1\| | | | | |
| | | | | hypothetical protein SORBIDRAFT_06g020130 | | | | |
| | | | | [Sorghum bicolor] | | | | |
| | | ADT92202 | 315259985 | N-acetylglucosaminyl-phosphatidylinositol | 0.912548 | Zea mays | 2895 | 4294 |
| | | | | de-N-acetylase-like protein [Zea mays] | | | | |
| | | BAJ97962 | 326488700 | predicted protein | 0.802281 | Hordeum vulgare subsp. vulgare | 2896 | 4295 |
| | | | | [Hordeum vulgare subsp. vulgare] | | | | |
| | | EEC78258 | 54362548 | hypothetical protein OsI_17935 | 0.787072 | Oryza sativa Indica Group | 2897 | |
| | | | | [Oryza sativa Indica Group] | | | | |
| | | NP_001054275 | 115461349 | Os04g0678800 [Oryza sativa Japonica Group] | 0.78327 | Oryza sativa Japonica Group | 2898 | 4296 |
| | | | | &gt:gi\|32487388\|emb\|CAE05722.1\| | | | | |
| | | | | OSJNBb0017I01.2 [Oryza sativa Japonica Group] | | | | |
| | | | | &gt:gi\|113565846\|dbj\|BAF16189.1\| Os04g0678800 | | | | |
| | | | | [Oryza sativa Japonica Group] | | | | |
| | | | | &gt:gi\|215701450\|dbj\|BAG92874.1\| unnamed | | | | |
| | | | | protein product [Oryza sativa Japonica Group] | | | | |
| | | | | &gt:gi\|215704470\|dbj\|BAG93904.1\| unnamed | | | | |
| | | | | protein product [Oryza sativa Japonica Group] | | | | |
| | | | | &gt:gi\|222629778\|gb\|EEE61910.1\| hypothetical | | | | |
| | | | | protein OsJ_16635 [Oryza sativa Japonica Group] | | | | |
| | 535-553 | ACF85503 | 194702837 | unknown [Zea mays] | 1 | Zea mays | 2899 | 4297 |
| | | | | &gt:gi\|223949391\|gb\|ACN28779.1\| unknown | | | | |
| | | | | [Zea mays] | | | | |
| | | NP_001149059 | 226497141 | LOC100282679 [Zea mays] | 0.948718 | Zea mays | 2900 | 4298 |
| | | | | &gt:gi\|195624404\|gb\|ACG34032.1\| | | | | |
| | | | | chaperone protein dnaJ [Zea mays] | | | | |
| | | ACF86076 | 194703983 | unknown [Zea mays] | 0.95338 | Zea mays | 2901 | 4299 |
| | | XP_002437726 | 242094471 | hypothetical protein SORBIDRAFT_10g001410 | 0.909091 | Sorghum bicolor | 2902 | 4300 |
| | | | | [Sorghum bicolor] | | | | |
| | | | | &gt:gi\|241915949\|gb\|EER89093.1\| | | | | |
| | | | | hypothetical protein SORBIDRAFT_10g001410 | | | | |
| | | | | [Sorghum bicolor] | | | | |
| | | NP_001056623 | 115466047 | Os06g0116800 [Oryza sativa Japonica Group] | 0.778555 | Oryza sativa Japonica Group | 2903 | 4301 |
| | | | | &gt:gi\|55296201\|dbj\|BAD67919.1\| putative | | | | |
| | | | | GFA2 [Oryza sativa Japonica Group] | | | | |
| | | | | &gt:gi\|113594663\|dbj\|BAF18537.1\| Os06g0116800 | | | | |
| | | | | [Oryza sativa Japonica Group] | | | | |
| | | | | &gt:gi\|215765163\|dbj\|BAG86860.1\| unnamed | | | | |
| | | | | protein product [Oryza sativa Japonica Group] | | | | |
| | | | | &gt:gi\|218197458\|gb\|EEC79885.1\| hypothetical | | | | |
| | | | | protein OsI_21393 [Oryza sativa Indica Group] | | | | |
| | | | | &gt:gi\|222637666\|gb\|EEE67798.1\| hypothetical | | | | |
| | | | | protein OsJ_25536 [Oryza sativa Japonica Group] | | | | |
| | 275-293 | XP_002437665 | 242094349 | hypothetical protein SORBIDRAFT_10g000420 | 1 | Sorghum bicolor | 2904 | 4302 |
| | | | | [Sorghum bicolor] | | | | |
| | | | | &gt:gi\|241915888\|gb\|EER89032.1\| | | | | |
| | | | | hypothetical protein SORBIDRAFT_10g000420 | | | | |
| | | | | [Sorghum bicolor] | | | | |
| | | ACN30602 | 223972828 | unknown [Zea mays] | 0.838323 | Zea mays | 2905 | 4303 |
| | | NP_001150955 | 226491174 | glutamine cyclotransferase [Zea mays] | 0.835329 | Zea mays | 2906 | 4304 |
| | | | | &gt:gi\|195643198\|gb\|ACG41067.1\| glutamine | | | | |
| | | | | cyclotransferase precursor [Zea mays] | | | | |
| | | NP_001056544 | 115465889 | Os06g0103700 [Oryza sativa Japonica Group] | 0.736527 | Oryza sativa Japonica Group | 2907 | 4305 |
| | | | | &gt:gi\|55296762\|dbj\|BAD67954.1\| putative | | | | |
| | | | | glutamine cyclotransferase precursor | | | | |
| | | | | [Oryza sativa Japonica Group] | | | | |
| | | | | &gt:gi\|113594584\|dbj\|BAF18458.1\| | | | | |
| | | | | Os06g0103700 [Oryza sativa Japonica Group] | | | | |
| | | | | &gt:gi\|215687230\|dbj\|BAG91795.1\| unnamed | | | | |
| | | | | protein product [Oryza sativa Japonica Group] | | | | |
| | 103-121 | ACL52777 | 219884804 | unknown [Zea mays] | 1 | Zea mays | 2908 | 4306 |
| | | XP_002453425 | 242064271 | hypothetical protein SORBIDRAFT_04g005820 | 0.819588 | Sorghum bicolor | 2909 | 4307 |
| | | | | [Sorghum bicolor] | | | | |
| | | | | &gt:gi\|241933256\|gb\|EES06401.1\| | | | | |
| | | | | hypothetical protein SORBIDRAFT_04g005820 | | | | |
| | | | | [Sorghum bicolor] | | | | |
| | 315-333 | NP_001150068 | 226506409 | N-acetylglucosaminyl-phosphatidylinositol | 1 | Zea mays | 2910 | 4308 |
| | | | | de-N-acetylase [Zea mays] | | | | |
| | | | | &gt:gi\|195636450\|gb\|ACG37693.1\| | | | | |
| | | | | N-acetylglucosaminyl-phosphatidylinositol | | | | |
| | | | | de-N-acetylase [Zea mays] | | | | |
| | 329-347 | NP_001140626 | 226493992 | hypothetical protein LOC100272700 [Zea mays] | 1 | Zea mays | 2911 | 4309 |
| | | | | &gt:gi\|194700230\|gb\|ACF84199.1\| unknown | | | | |
| | | | | [Zea mays] | | | | |
| | 315-333 | XP_002448044 | 242076215 | hypothetical protein SORBIDRAFT_06g020130 | 1 | Sorghum bicolor | 2912 | 4310 |
| | | | | [Sorghum bicolor] | | | | |
| | | | | &gt:gi\|241939227\|gb\|EES12372.1\| | | | | |
| | | | | hypothetical protein SORBIDRAFT_06g020130 | | | | |
| | | | | [Sorghum bicolor] | | | | |
| | 1361-1379 | ADT92191 | 315259978 | putative potassium efflux system | 1 | Zea mays | 2913 | |
| | | | | protein family [Zea mays] | | | | |
| | | ADT92192 | 315259985 | putative potassium efflux system | 0.990308 | Zea mays | 2914 | |
| | | | | family protein [Zea mays] | | | | |
| | | XP_002448794 | 242077715 | hypothetical protein SORBIDRAFT_06g033310 | 0.881938 | Sorghum bicolor | 2915 | 4311 |
| | | | | [Sorghum bicolor] | | | | |
| | | | | &gt:gi\|241939977\|gb\|EES13122.1\| | | | | |
| | | | | hypothetical protein SORBIDRAFT_06g033310 | | | | |
| | | | | [Sorghum bicolor] | | | | |
| | | NP_001054299 | 115461397 | Os04g0682800 [Oryza sativa Japonica Group] | 0.807048 | Oryza sativa Japonica Group | 2916 | 4312 |
| | | | | &gt:gi\|38345563\|emb\|CAE03437.2\| | | | | |
| | | | | OSJNBa0032F06.20 [Oryza sativa Japonica Group] | | | | |
| | | | | &gt:gi\|113565870\|dbj\|BAF16213.1\| Os04g0682800 | | | | |
| | | | | [Oryza sativa Japonica Group] | | | | |
| | | | | &gt:gi\|215768459\|dbj\|BAH00688.1\| unnamed | | | | |
| | | | | protein product [Oryza sativa Japonica Group] | | | | |
| | | BAJ87826 | 326513613 | predicted protein | 0.769163 | Hordeum vulgare subsp. vulgare | 2917 | 4313 |
| | | | | [Hordeum vulgare subsp. vulgare] | | | | |
| | | BAJ89683 | 326487397 | predicted protein | 0.768282 | Hordeum vulgare subsp. vulgare | 2918 | 4314 |
| | | | | [Hordeum vulgare subsp. vulgare] | | | | |
| | 38-56 | XP_002436836 | 242092691 | hypothetical protein SORBIDRAFT_10g009750 | 1 | Sorghum bicolor | 2919 | 4315 |
| | | | | [Sorghum bicolor] | | | | |
| | | | | &gt:gi\|241915059\|gb\|EER88203.1\| | | | | |
| | | | | hypothetical protein SORBIDRAFT_10g009750 | | | | |
| | | | | [Sorghum bicolor] | | | | |
| | 81-99 | NP_001143033 | 226529719 | hypothetical protein LOC100275501 [Zea mays] | 1 | Zea mays | 2920 | 4316 |
| | | | | &gt:gi\|195613282\|gb\|ACG28471.1\| hypothetical | | | | |
| | | | | protein [Zea mays] | | | | |
| | 2226-2244 | NP_001142912 | 226498003 | hypothetical protein LOC100275344 [Zea mays] | 1 | Zea mays | 2921 | 4317 |
| | | | | &gt:gi\|195611342\|gb\|ACG27501.1\| hypothetical | | | | |
| | | | | protein [Zea mays] | | | | |
| | | XP_002452898 | 242063217 | hypothetical protein SORBIDRAFT_04g034580 | 0.941261 | Sorghum bicolor | 2922 | 4318 |
| | | | | [Sorghum bicolor] | | | | |
| | | | | &gt:gi\|241932729\|gb\|EES05874.1\| | | | | |
| | | | | hypothetical protein SORBIDRAFT_04g034580 | | | | |
| | | | | [Sorghum bicolor] | | | | |
| | | BAK07849 | 326525157 | predicted protein | 0.84384 | Hordeum vulgare subsp. vulgare | 2923 | 4319 |
| | | | | [Hordeum vulgare subsp. vulgare] | | | | |
| | | Q69T58 | | RecName: Full = Homeobox-leucine zipper protein | 0.808023 | Oryza sativa Japonica Group | 2924 | |
| | | | | ROC8; AltName: Full = GLABRA 2-like homeobox | | | | |
| | | | | protein 8; AltName: Full = HD-ZIP protein ROC8; | | | | |
| | | | | AltName: Full = Homeodomain transcription factor | | | | |
| | | | | ROC8; AltName: Full = Protein RICE OUTERMOST | | | | |
| | | | | CELL-SPECIFIC 8 | | | | |
| | | BAD35894 | 51091189 | putative homeobox | 0.808023 | Oryza sativa Japonica Group | 2925 | 4320 |
| | | | | [Oryza sativa Japonica Group] | | | | |
| | | XP_002438026 | 242095071 | hypothetical protein SORBIDRAFT_10g006820 | 0.789398 | Sorghum bicolor | 2926 | 4321 |
| | | | | [Sorghum bicolor] | | | | |
| | | | | &gt:gi\|241916249\|gb\|EER89393.1\| | | | | |
| | | | | hypothetical protein SORBIDRAFT_10g006820 | | | | |
| | | | | [Sorghum bicolor] | | | | |
| | | EEC80207 | 54362548 | hypothetical protein OsI_22100 | 0.724928 | Oryza sativa Indica Group | 2927 | |
| | | | | [Oryza sativa Indica Group] | | | | |
| | 384-402 | NP_001146570 | 226496306 | hypothetical protein LOC100280166 [Zea mays] | 1 | Zea mays | 2928 | 4322 |
| | | | | &gt:gi\|219887859\|gb\|ACL54304.1\| unknown | | | | |
| | | | | [Zea mays] | | | | |
| | | NP_001063308 | 297609549 | Os09g0446200 [Oryza sativa Japonica Group] | 0.924125 | Oryza sativa Japonica Group | 2929 | 4323 |
| | | | | &gt:gi\|255678935\|dbj\|BAF25222.2\| Os09g0446200 | | | | |
| | | | | [Oryza sativa Japonica Group] | | | | |
| | | EEC84667 | 54362548 | hypothetical protein OsI_31569 | 0.815175 | Oryza sativa Indica Group | 2930 | |
| | | | | [Oryza sativa Indica Group] | | | | |
| Predicted | 443-462 | XP_002454541 | 242066503 | hypothetical protein SORBIDRAFT_04g032980 | 1 | Sorghum bicolor | 2931 | 4324 |
| siRNA | | | | [Sorghum bicolor] | | | | |
| 59211 | | | | &gt:gi\|241934372\|gb\|EES07517.1\| | | | | |
| | | | | hypothetical protein SORBIDRAFT_04g032980 | | | | |
| | | | | [Sorghum bicolor] | | | | |
| | | ACR36020 | 238009969 | unknown [Zea mays] | 0.951667 | Zea mays | 2932 | 4325 |
| | | NP_001147126 | 226494332 | cell division cycle protein 23 [Zea mays] | 0.948333 | Zea mays | 2933 | 4326 |
| | | | | &gt:gi\|195607482\|gb\|ACG25571.1\| cell | | | | |
| | | | | division cycle protein 23 [Zea mays] | | | | |
| | | EEC73723 | 54362548 | hypothetical protein OsI_08332 | 0.878333 | Oryza sativa Indica Group | 2934 | |
| | | | | [Oryza sativa Indica Group] | | | | |
| | | NP_001047624 | 115447688 | Os02g0656300 [Oryza sativa Japonica Group] | 0.876667 | Oryza sativa Japonica Group | 2935 | 4327 |
| | | | | &gt:gi\|49388560\|dbj\|BAD25679.1\| putative | | | | |
| | | | | cell division cycle protein 23 | | | | |
| | | | | [Oryza sativa Japonica Group] | | | | |
| | | | | &gt:gi\|113537155\|dbj\|BAF09538.1\| | | | | |
| | | | | Os02g0656300 [Oryza sativa Japonica Group] | | | | |
| | | | | &gt:gi\|222623375\|gb\|EEE57507.1\| | | | | |
| | | | | hypothetical protein OsJ_07790 | | | | |
| | | | | [Oryza sativa Japonica Group] | | | | |
| | | CAC39070 | 14140112 | anaphase-promoting complex subunit | 0.875 | Oryza sativa | 2936 | 4328 |
| | | | | 8-like protein [Oryza sativa] | | | | |
| | | BAK03657 | 326517476 | predicted protein | 0.885 | Hordeum vulgare subsp. vulgare | 2937 | 4329 |
| | | | | [Hordeum vulgare subsp. vulgare] | | | | |
| | | XP_002266966 | 225463150 | PREDICTED: hypothetical protein | 0.701667 | Vitis vinifera | 2938 | 4330 |
| | | | | [Vitis vinifera] | | | | |
| | | CAN83330 | 147844944 | hypothetical protein VITISV_005847 | 0.701667 | Vitis vinifera | 2939 | 4331 |
| | | | | [Vitis vinifera] | | | | |
| | 1053-1072 | XP_002438417 | 242095853 | hypothetical protein SORBIDRAFT_10g017800 | 1 | Sorghum bicolor | 2940 | 4332 |
| | | | | [Sorghum bicolor] | | | | |
| | | | | &gt:gi\|241916640\|gb\|EER89784.1\| | | | | |
| | | | | hypothetical protein SORBIDRAFT_10g017800 | | | | |
| | | | | [Sorghum bicolor] | | | | |
| | | NP_001141152 | 226494465 | hypothetical protein LOC100273238 [Zea mays] | 0.92435 | Zea mays | 2941 | 4333 |
| | | | | &gt:gi\|194702930\|gb\|ACF85549.1\| unknown | | | | |
| | | | | [Zea mays] | | | | |
| | | ACF85911 | 194703653 | unknown [Zea mays] | 0.898345 | Zea mays | 2942 | 4334 |
| | | NP_001057632 | 115468065 | Os06g0474500 [Oryza sativa Japonica Group] | 0.869976 | Oryza sativa Japonica Group | 2943 | 4335 |
| | | | | &gt:gi\|51090811\|dbj\|BAD35288.1\| putative | | | | |
| | | | | ZmEBE-1 protein [Oryza sativa Japonica Group] | | | | |
| | | | | &gt:gi\|113595672\|dbj\|BAF19546.1\| Os06g0474500 | | | | |
| | | | | [Oryza sativa Japonica Group] | | | | |
| | | | | &gt:gi\|215706920\|dbj\|BAG93380.1\| unnamed | | | | |
| | | | | protein product [Oryza sativa Japonica Group] | | | | |
| | | | | &gt:gi\|215740902\|dbj\|BAG97058.1\| unnamed | | | | |
| | | | | protein product [Oryza sativa Japonica Group] | | | | |
| | | | | &gt:gi\|222635579\|gb\|EEE65711.1\| hypothetical | | | | |
| | | | | protein OsJ_21346 [Oryza sativa Japonica Group] | | | | |
| | | EAZ00932 | 54362548 | hypothetical protein OsI_22962 | 0.867612 | Oryza sativa Indica Group | 2944 | |
| | | | | [Oryza sativa Indica Group] | | | | |
| Predicted | 528-547 | NP_001152385 | 226507515 | lyase [Zea mays] | 1 | Zea mays | 2945 | 4336 |
| siRNA | | | | &gt:gi\|195655769\|gb\|ACG47352.1\| | | | | |
| 59300 | | | | lyase [Zea mays] | | | | |
| | | XP_002441731 | 242082611 | hypothetical protein SORBIDRAFT_08g001440 | 0.766575 | Sorghum bicolor | 2946 | 4337 |
| | | | | [Sorghum bicolor] | | | | |
| | | | | &gt:gi\|241942424\|gb\|EES15569.1\| | | | | |
| | | | | hypothetical protein SORBIDRAFT_08g001440 | | | | |
| | | | | [Sorghum bicolor] | | | | |
| | 1224-1243 | XP_002445025 | 242080512 | hypothetical protein SORBIDRAFT_07g003020 | 1 | Sorghum bicolor | 2947 | 4338 |
| | | | | [Sorghum bicolor] | | | | |
| | | | | &gt:gi\|241941375\|gb\|EES14520.1\| | | | | |
| | | | | hypothetical protein SORBIDRAFT_07g003020 | | | | |
| | | | | [Sorghum bicolor] | | | | |
| | | NP_001146372 | 226528117 | hypothetical protein LOC100279950 [Zea mays] | 0.892514 | Zea mays | 2948 | 4339 |
| | | | | &gt:gi\|219886865\|gb\|ACL53807.1\| unknown | | | | |
| | | | | [Zea mays] | | | | |
| | | XP_002445026 | 242080514 | hypothetical protein SORBIDRAFT_07g003040 | 0.869482 | Sorghum bicolor | 2949 | 4340 |
| | | | | [Sorghum bicolor] & | | | | |
| | | | | gt:gi\|241941376\|gb\|EES14521.1\| | | | | |
| | | | | hypothetical protein SORBIDRAFT_07g003040 | | | | |
| | | | | [Sorghum bicolor] | | | | |
| | | NP_001169175 | 293335560 | hypothetical protein LOC100383025 [Zea mays] | 0.856046 | Zea mays | 2950 | 4341 |
| | | | | &gt:gi\|223975313\|gb\|ACN31844.1\| unknown | | | | |
| | | | | [Zea mays] | | | | |
| | | | | &gt:gi\|223975749\|gb\|ACN32062.1\| | | | | |
| | | | | unknown [Zea mays] | | | | |
| | | BAD11769 | 42794043 | tryptophan decarboxylase [Hordeum vulgare] | 0.809981 | Hordeum vulgare | 2951 | 4342 |
| | | NP_001060969 | 115474744 | Os08g0140500 [Oryza sativa Japonica Group] | 0.829175 | Oryza sativa Japonica Group | 2952 | 4343 |
| | | | | &gt:gi\|42761330\|dbj\|BAD11583.1\| putative | | | | |
| | | | | Aromatic-L-amino-acid decarboxylase | | | | |
| | | | | [Oryza sativa Japonica Group] | | | | |
| | | | | &gt:gi\|45736124\|dbj\|BAD13170.1\| putative | | | | |
| | | | | Aromatic-L-amino-acid decarboxylase | | | | |
| | | | | [Oryza sativa Japonica Group] | | | | |
| | | | | &gt:gi\|113622938\|dbj\|BAF22883.1\| | | | | |
| | | | | Os08g0140500 [Oryza sativa Japonica Group] | | | | |
| | | | | &gt:gi\|125560099\|gb\|EAZ05547.1\| hypothetical | | | | |
| | | | | protein OsI_27762 [Oryza sativa Indica Group] | | | | |
| | | BAD11768 | 42794041 | tryptophan decarboxylase | 0.809981 | Hordeum vulgare subsp. spontaneum | 2953 | 4344 |
| | | | | [Hordeum vulgare subsp. spontaneum] | | | | |
| | | NP_001060968 | 115474742 | Os08g0140300 [Oryza sativa Japonica Group] | 0.821497 | Oryza sativa Japonica Group | 2954 | 4345 |
| | | | | &gt:gi\|42761328\|dbj\|BAD11581.1\| putative | | | | |
| | | | | Aromatic-L-amino-acid decarboxylase | | | | |
| | | | | [Oryza sativa Japonica Group] | | | | |
| | | | | &gt:gi\|113622937\|dbj\|BAF22882.1\| | | | | |
| | | | | Os08g0140300 [Oryza sativa Japonica Group] | | | | |
| | | | | &gt:gi\|125560097\|gb\|EAZ05545.1\| hypothetical | | | | |
| | | | | protein OsI_27760 [Oryza sativa Indica Group] | | | | |
| | | | | &gt:gi\|125602145\|gb\|EAZ41470.1\| hypothetical | | | | |
| | | | | protein OsJ_25993 [Oryza sativa Japonica Group] | | | | |
| | | | | &gt:gi\|215697229\|dbj\|BAG91223.1\| unnamed | | | | |
| | | | | protein product [Oryza sativa Japonica Group] | | | | |
| | | ADE48535 | 293612213 | putative decarboxylase protein | 0.744722 | Triticum aestivum | 2955 | |
| | | | | [Triticum aestivum] | | | | |
| | 94-113 | BAJ97340 | 326528636 | predicted protein | 1 | Hordeum vulgare subsp. spontaneum | 2956 | 4346 |
| | | | | [Hordeum vulgare subsp. vulgare] | | | | |
| | | NP_001168594 | 293334714 | hypothetical protein LOC100382378 [Zea mays] | 0.872204 | Zea mays | 2957 | 4347 |
| | | | | &gt:gi\|223949419\|gb\|ACN28793.1\| unknown | | | | |
| | | | | [Zea mays] | | | | |
| | | A2YGP6 | | RecName: Full = UDP-sugar pyrophosphorylase | 0.865815 | Oryza sativa Indica Group | 2958 | |
| | | NP_001058482 | 115469765 | Os06g0701200 [Oryza sativa Japonica Group] | 0.84984 | Oryza sativa Japonica Group | 2959 | 4348 |
| | | | | &gt:gi\|75112500\|sp\|Q5Z8Y4.1\|USP_ORYSJ | | | | |
| | | | | RecName: Full = UDP-sugar pyrophosphorylase | | | | |
| | | | | &gt:gi\|53792734\|dbj\|BAD53770.1\| | | | | |
| | | | | UDP-N-acetylglucosamine pyrophosphorylase-like | | | | |
| | | | | [Oryza sativa Japonica Group] | | | | |
| | | | | &gt:gi\|113596522\|dbj\|BAF20396.1\| Os06g0701200 | | | | |
| | | | | [Oryza sativa Japonica Group] | | | | |
| | | | | &gt:gi\|215686708\|dbj\|BAG88961.1\| unnamed | | | | |
| | | | | protein product [Oryza sativa Japonica Group] | | | | |
| | | NP_001152310 | 226501637 | LOC100285949 [Zea mays] | 0.84984 | Zea mays | 2960 | 4349 |
| | | | | &gt:gi\|195654965\|gb\|ACG46950.1\| | | | | |
| | | | | UDP-sugar pyrophospharylase [Zea mays] | | | | |
| | | EEC81262 | 54362548 | hypothetical protein OsI_24356 | 0.84984 | Oryza sativa Indica Group | 2961 | |
| | | | | [Oryza sativa Indica Group] | | | | |
| | | EEE66302 | 54398660 | hypothetical protein OsJ_22533 | 0.833866 | Oryza sativa Japonica Group | 2962 | |
| | | | | [Oryza sativa Japonica Group] | | | | |
| | | Q0GZS3 | | RecName: Full = UDP-sugar pyrophospharylase; | 0.72524 | Cucumis melo | 2963 | |
| | | | | AltName: Full = UDP-galaclose/glucose | | | | |
| | | | | pyrophosphorylase; Short = UGGPase | | | | |
| | | | | &gt:gi\|88954061\|gb\|ABD59006.1\| | | | | |
| | | | | UDP-galactose/glucose pyrophosphorylase | | | | |
| | | | | [Cucumis melo] | | | | |
| | | XP_002864182 | 297792594 | hypothetical protein ARALYDRAFT_495327 | 0.71885 | Arabidopsis lyrata subsp. lyrata | 2964 | 4350 |
| | | | | [Arabidopsis lyrata subsp. lyrata] | | | | |
| | | | | &gt:gi\|297310017\|gb\|EFH40441.1\| hypothetical | | | | |
| | | | | protein ARALYDRAFT_495327 | | | | |
| | | | | [Arabidopsis lyrata subsp. lyrata] | | | | |
| | | NP_568775 | 145359167 | UDP-sugar pyrophosphorylase [Arabidopsis thaliana] | 0.722045 | Arabidopsis thaliana | 2965 | 4351 |
| | | | | &gt:gi\|75168956\|sp\|Q9C5I1.1\|USP_ARATH | | | | |
| | | | | RecName: Full = UDP-sugar pyrophosphorylase; | | | | |
| | | | | Short = AtUSP | | | | |
| | | | | &gt:gi\|13430648\|gb\|AAK25946.1\|AF360236_1 | | | | |
| | | | | unknown protein [Arabidopsis thaliana] | | | | |
| | | | | &gt:gi\|14532822\|gb\|AAK64093.1\| unknown | | | | |
| | | | | protein [Arabidopsis thaliana] | | | | |
| | | | | &gt:gi\|84181457\|gb\|ABC55066.1\| nonspecific | | | | |
| | | | | UDP-sugar pyrophosphorylase | | | | |
| | | | | [Arabidopsis thaliana] | | | | |
| | | | | &gt:gi\|332008851\|gb\|AED96234.1\| | | | | |
| | | | | UDP-sugar pyrophosphorylase | | | | |
| | | | | [Arabidopsis thaliana] | | | | |
| | 1268-1287 | XP_002443799 | 242078060 | hypothetical protein SORBIDRAFT_07g002260 | 1 | Sorghum bicolor | 2966 | 4352 |
| | | | | [Sorghum bicolor] | | | | |
| | | | | &gt:gi\|241940149\|gb\|EES13294.1\| | | | | |
| | | | | hypothetical protein SORBIDRAFT_07g002260 | | | | |
| | | | | [Sorghum bicolor] | | | | |
| | | NP_001170379 | 293334010 | hypothetical protein LOC100384362 [Zea mays] | 0.918103 | Zea mays | 2967 | 4353 |
| | | | | &gt:gi\|224035469\|gb\|ACN36810.1\| unknown | | | | |
| | | | | [Zea mays] | | | | |
| | | XP_002454589 | 242066599 | hypothetical protein SORBIDRAFT_04g033960 | 0.851293 | Sorghum bicolor | 2968 | 4354 |
| | | | | [Sorghum bicolor] | | | | |
| | | | | &gt:gi\|241934420\|gb\|EES07565.1\| | | | | |
| | | | | hypothetical protein SORBIDRAFT_04g033960 | | | | |
| | | | | [Sorghum bicolor] | | | | |
| | | XP_002443797 | 242078056 | hypothetical protein SORBIDRAFT_07g002250 | 0.788793 | Sorghum bicolor | 2969 | 4355 |
| | | | | [Sorghum bicolor] | | | | |
| | | | | &gt:gi\|241940147\|gb\|EES13292.1\| | | | | |
| | | | | hypothetical protein SORBIDRAFT_07g002250 | | | | |
| | | | | [Sorghum bicolor] | | | | |
| | | NP_001147827 | 226502679 | LHT1 [Zea mays] | 0.784483 | Zea mays | 2970 | 4356 |
| | | | | &gt:gi\|195613982\|gb\|ACG28821.1\| | | | | |
| | | | | LHT1 [Zea mays] | | | | |
| | | NP_001152139 | 226498595 | LHT1 [Zea mays] | 0.75431 | Zea mays | 2971 | 4357 |
| | | | | &gt:gi\|195653153\|gb\|ACG46044.1\| | | | | |
| | | | | LHT1 [Zea mays] | | | | |
| | | CAD89802 | 30409135 | histidine amino acid transporter | 0.784483 | Oryza sativa Indica Group | 2972 | 4358 |
| | | | | [Oryza sativa Indica Group] | | | | |
| | | XP_002443796 | 242078054 | hypothetical protein SORBIDRAFT_07g002240 | 0.75431 | Sorghum bicolor | 2973 | 4359 |
| | | | | [Sorghum bicolor] | | | | |
| | | | | &gt:gi\|241940146\|gb\|EES13291.1\| | | | | |
| | | | | hypothetical protein SORBIDRAFT_07g002240 | | | | |
| | | | | [Sorghum bicolor] | | | | |
| | 241-260 | NP_001144625 | 226498917 | hypothetical protein LOC100277643 [Zea mays] | 1 | Zea mays | 2974 | 4360 |
| | | | | &gt:gi\|195644788\|gb\|ACG41862.1\| hypothetical | | | | |
| | | | | protein [Zea mays] | | | | |
| | | BAJ94382 | 326494525 | predicted protein | 0.762195 | Hordeum vulgare subsp. vulgare | 2975 | 4361 |
| | | | | [Hordeum vulgare subsp. vulgare] | | | | |
| | | NP_001047900 | 115448240 | Os02g0711100 [Oryza sativa Japonica Group] | 0.75 | Oryza sativa Japonica Group | 2976 | 4362 |
| | | | | &gt:gi\|41052650\|dbj\|BAD07498.1\| pentatricopeptide | | | | |
| | | | | (PPR) repeat-containing protein-like | | | | |
| | | | | [Oryza sativa Japonica Group] | | | | |
| | | | | &gt:gi\|41052866\|dbj\|BAD07780.1\| pentatricopeptide | | | | |
| | | | | (PPR) repeat-containing protein-like | | | | |
| | | | | [Oryza sativa Japonica Group] | | | | |
| | | | | &gt:gi\|113537431\|dbj\|BAF09814.1\| Os02g0711100 | | | | |
| | | | | [Oryza sativa Japonica Group] | | | | |
| | | | | &gt:gi\|125540864\|gb\|EAY87259.1\| hypothetical | | | | |
| | | | | protein OsI_08660 [Oryza sativa Indica Group] | | | | |
| | | | | &gt:gi\|215687282\|dbj\|BAG91847.1\| unnamed | | | | |
| | | | | protein product [Oryza sativa Japonica Group] | | | | |
| | 1395-1414 | XP_002458633 | 242058974 | hypothetical protein SORBIDRAFT_03g037090 | 1 | Sorghum bicolor | 2977 | 4363 |
| | | | | [Sorghum bicolor] | | | | |
| | | | | &gt:gi\|241930608\|gb\|EES03753.1\| | | | | |
| | | | | hypothetical protein SORBIDRAFT_03g037090 | | | | |
| | | | | [Sorghum bicolor] | | | | |
| | | NP_001146020 | 226507335 | hypothetical protein LOC100279551 [Zea mays] | 0.858388 | Zea mays | 2978 | 4364 |
| | | | | &gt:gi\|219885345\|gb\|ACL53047.1\| unknown | | | | |
| | | | | [Zea mays] | | | | |
| | | BAJ95510 | 326505677 | predicted protein | 0.840959 | Hordeum vulgare subsp. vulgare | 2979 | 4365 |
| | | | | [Hordeum vulgare subsp. vulgare] | | | | |
| | | NP_001142614 | 226529843 | hypothetical protein LOC100274883 [Zea mays] | 0.814815 | Zea mays | 2980 | 4366 |
| | | | | &gt:gi\|224030957\|gb\|ACN34554.1\| unknown | | | | |
| | | | | [Zea mays] | | | | |
| | | AAX96247 | 45592979 | hypothetical protein LOC_Os11g25920 | 0.795207 | Oryza sativa Japonica Group | 2981 | |
| | | | | [Oryza sativa Japonica Group] | | | | |
| | | EEC83703 | 54362548 | hypothetical protein OsI_29522 | 0.786492 | Oryza sativa Indica Group | 2982 | |
| | | | | [Oryza sativa Indica Group] | | | | |
| Predicted | 50-67 | NP_001149733 | 226509895 | UBA and UBX domain-containing protein [Zea mays] | 1 | Zea mays | 2983 | 4367 |
| siRNA | | | | &gt:gi\|195629900\|gb\|ACG36591.1\| UBA and UBX | | | | |
| 59379 | | | | domain-containing protein [Zea mays] | | | | |
| | | XP_002446630 | 242073387 | hypothetical protein SORBIDRAFT_06g019230 | 0.926282 | Sorghum bicolor | 2984 | 4368 |
| | | | | [Sorghum bicolor] | | | | |
| | | | | &gt:gi\|241937813\|gb\|EES10958.1\| | | | | |
| | | | | hypothetical protein SORBIDRAFT_06g019230 | | | | |
| | | | | [Sorghum bicolor] | | | | |
| | | ACN31131 | 223973886 | unknown [Zea mays] | 0.807692 | Zea mays | 2985 | 4369 |
| | | NP_001148755 | 226496278 | LOC100282371 [Zea mays] | 0.910256 | Zea mays | 2986 | 4370 |
| | | | | &gt:gi\|195621900\|gb\|ACG32780.1\| UBA and UBX | | | | |
| | | | | domain-containing protein [Zea mays] | | | | |
| | | NP_001053018 | 115458835 | Os04g0464500 [Oryza sativa Japonica Group] | 0.753205 | Oryza sativa Japonica Group | 2987 | 4371 |
| | | | | &gt:gi\|113564589\|dbj\|BAF14932.1\| Os04g0464500 | | | | |
| | | | | [Oryza sativa Japonica Group] | | | | |
| | | CAE04359 | 32489876 | OSJNBa0060P14.10 [Oryza sativa Japonica Group] | 0.740385 | Oryza sativa Japonica Group | 2988 | 4372 |
| | | | | &gt:gi\|125548622\|gb\|EAY94444.1\| hypothetical | | | | |
| | | | | protein OsI_16215 [Oryza sativa Indica Group] | | | | |
| | | | | &gt:gi\|125590658\|gb\|EAZ31008.1\| hypothetical | | | | |
| | | | | protein OsJ_15090 [Oryza sativa Japonica Group] | | | | |
| Predicted | 83-101 | XP_002455291 | 242052290 | hypothetical protein SORBIDRAFT_03g007910 | 1 | Sorghum bicolor | 2989 | 4373 |
| siRNA | | | | [Sorghum bicolor] | | | | |
| 59474 | | | | &gt:gi\|241927266\|gb\|EES00411.1\| | | | | |
| | | | | hypothetical protein SORBIDRAFT_03g007910 | | | | |
| | | | | [Sorghum bicolor] | | | | |
| | | NP_001130987 | 212275100 | hypothetical protein LOC100192092 [Zea mays] | 0.7813 | Zea mays | 2990 | 4374 |
| | | | | &gt:gi\|194690638\|gb\|ACF79403.1\| unknown | | | | |
| | | | | [Zea mays] | | | | |
| | | | | &gt:gi\|224030713\|gb\|ACN34432.1\| | | | | |
| | | | | unknown [Zea mays] | | | | |
| | | NP_001130824 | 212275671 | hypothetical protein LOC100191928 [Zea mays] | 0.73851 | Zea mays | 2991 | 4375 |
| | | | | &gt:gi\|194690210\|gb\|ACF79189.1\| unknown | | | | |
| | | | | [Zea mays] | | | | |
| | | ACN27042 | 223945916 | unknown [Zea mays] | 0.787639 | Zea mays | 2992 | 4376 |
| | | NP_001130385 | 212275649 | hypothetical protein LOC100191481 [Zea mays] | 0.786054 | Zea mays | 2993 | 4377 |
| | | | | &gt:gi\|194688994\|gb\|ACF78581.1\| unknown | | | | |
| | | | | [Zea mays] | | | | |
| | | ACG48121 | 195657306 | glycosyltransferase [Zea mays] | 0.784469 | Zea mays | 2994 | 4378 |
| | 267-285 | XP_002466400 | 242038010 | hypothetical protein SORBIDRAFT_01g007120 | 1 | Sorghum bicolor | 2995 | 4379 |
| | | | | [Sorghum bicolor] | | | | |
| | | | | &gt:gi\|241920254\|gb\|EER93398.1\| | | | | |
| | | | | hypothetical protein SORBIDRAFT_01g007120 | | | | |
| | | | | [Sorghum bicolor] | | | | |
| | | ACF79259 | 194690349 | unknown [Zea mays] | 0.989071 | Zea mays | 2996 | 4380 |
| | | NP_001130882 | 212275449 | hypothetical protein LOC100191986 [Zea mays] | 0.986339 | Zea mays | 2997 | 4381 |
| | | | | &gt:gi\|195622040\|gb\|ACG32850.1\| | | | | |
| | | | | serine/threonine-protein kinase SAPK8 | | | | |
| | | | | [Zea mays] | | | | |
| | | ACL52689 | 219884628 | unknown [Zea mays] | 0.978142 | Zea mays | 2998 | 4382 |
| | | BAK05495 | 326487645 | predicted protein | 0.945355 | Hordeum vulgare subsp. vulgare | 2999 | 4383 |
| | | | | [Hordeum vulgare subsp. vulgare] | | | | |
| | | NP_001051371 | 115455540 | Os03g0764800 [Oryza sativa Japonica Group] | 0.934426 | Oryza sativa Japonica Group | 3000 | 4384 |
| | | | | &gt:gi\|71153747\|sp\|Q7Y0B9.1\|SAPK8_ORYSJ | | | | |
| | | | | RecName: Full = Serine/threonine-protein kinase | | | | |
| | | | | SAPK8; AltName: Full = Osmotic stress/abscisic | | | | |
| | | | | acid-activated protein kinase 8 | | | | |
| | | | | &gt:gi\|31415944\|gb\|AAP50965.1\| | | | | |
| | | | | putative serine-threonine protein kinase | | | | |
| | | | | [Oryza sativa Japonica Group] | | | | |
| | | | | &gt:gi\|46917344\|dbj\|BAD18004.1\| | | | | |
| | | | | serine/threonine protein kinase SAPK8 | | | | |
| | | | | [Oryza sativa Japonica Group] | | | | |
| | | | | &gt:gi\|108711239\|gb\|ABF99034.1\| | | | | |
| | | | | Serine/threonine-protein kinase SAPK9, | | | | |
| | | | | putative, expressed [Oryza sativa Japonica Group] | | | | |
| | | | | &gt:gi\|113549842\|dbj\|BAF13285.1\| Os03g0764800 | | | | |
| | | | | [Oryza sativa Japonica Group] | | | | |
| | | CAN62745 | 147788087 | hypothetical protein VITISV_025025 | 0.836066 | Vitis vinifera | 3001 | |
| | | | | [Vitis vinifera] | | | | |
| | | XP_002284959 | 225428694 | PREDICTED: hypothetical protein [Vitis vinifera] | 0.836066 | Vitis vinifera | 3002 | 4385 |
| | | | | &gt:gi\|297741336\|emb\|CBI32467.3\| unnamed protein | | | | |
| | | | | product [Vitis vinifera] | | | | |
| | 612-630 | XP_002440597 | 242089528 | hypothetical protein SORBIDRAFT_09g003790 | 1 | Sorghum bicolor | 3003 | 4386 |
| | | | | [Sorghum bicolor] | | | | |
| | | | | &gt:gi\|241945882\|gb\|EES19027.1\| | | | | |
| | | | | hypothetical protein SORBIDRAFT_09g003790 | | | | |
| | | | | [Sorghum bicolor] | | | | |
| | | XP_002439286 | 242086906 | hypothetical protein SORBIDRAFT_09g003800 | 0.933544 | Sorghum bicolor | 3004 | 4387 |
| | | | | [Sorghum bicolor] | | | | |
| | | | | &gt:gi\|3386565\|gb\|AAC28488.1\| | | | | |
| | | | | 1-aminocyclopropane-1-carboxylate oxidase | | | | |
| | | | | [Sorghum bicolor] | | | | |
| | | | | &gt:gi\|241944571\|gb\|EES17716.1\| | | | | |
| | | | | hypothetical protein SORBIDRAFT_09g003800 | | | | |
| | | | | [Sorghum bicolor] | | | | |
| | | AAR25561 | 38607362 | acc oxidase [Zea mays] | 0.933544 | Zea mays | 3005 | 4388 |
| | | NP_001105234 | 162458172 | acc oxidase [Zea mays] | 0.93038 | Zea mays | 3006 | 4389 |
| | | | | &gt:gi\|38607365\|gb\|AAR25562.1\| | | | | |
| | | | | acc oxidase [Zea mays] | | | | |
| | | NP_001141367 | 226495452 | hypothetical protein LOC100273458 [Zea mays] | 0.93038 | Zea mays | 3007 | 4390 |
| | | | | &gt:gi\|194704206\|gb\|ACF86187.1\| unknown | | | | |
| | | | | [Zea mays] | | | | |
| | | BAK00849 | 326526920 | predicted protein | 0.835443 | Hordeum vulgare subsp. vulgare | 3008 | 4391 |
| | | | | [Hordeum vulgare subsp. vulgare] | | | | |
| | | BAJ87146 | 326509860 | predicted protein [Hordeum vulgare subsp. vulgare] | 0.825949 | Hordeum vulgare subsp. vulgare | 3009 | 4392 |
| | | | | &gt:gi\|326510641\|dbj\|BAJ87537.1\| predicted | | | | |
| | | | | protein [Hordeum vulgare subsp. vulgare] | | | | |
| | | NP_001054657 | 115462114 | Os05g0149400 [Oryza sativa Japonica Group] | 0.794304 | Oryza sativa Japonica Group | 3010 | 4393 |
| | | | | &gt:gi\|52353464\|gb\|AAU44031.1\| putative | | | | |
| | | | | 1-aminocyclopropane-1-carboxylate oxidase | | | | |
| | | | | [Oryza sativa Japonica Group] | | | | |
| | | | | &gt:gi\|113578208\|dbj\|BAF16571.1\| Os05g0149400 | | | | |
| | | | | [Oryza sativa Japonica Group] | | | | |
| | | | | &gt:gi\|215686421\|dbj\|BAG87706.1\| unnamed | | | | |
| | | | | protein product [Oryza sativa Japonica Group] | | | | |
| | | | | &gt:gi\|218196096\|gb\|EEC78523.1\| hypothetical | | | | |
| | | | | protein OsI_18467 [Oryza sativa Indica Group] | | | | |
| | 595-613 | NP_001151137 | 226502371 | LOC100284770 [Zea mays] | 1 | Zea mays | 3011 | 4394 |
| | | | | &gt:gi\|195644530\|gb\|ACG41733.1\| anthranilate | | | | |
| | | | | N-benzoyltransferase protein 1 [Zea mays] | | | | |
| | | XP_002462399 | 242049309 | hypothetical protein SORBIDRAFT_02g025010 | 0.896074 | Sorghum bicolor | 3012 | 4395 |
| | | | | [Sorghum bicolor] | | | | |
| | | | | &gt:gi\|241925776\|gb\|EER98920.1\| | | | | |
| | | | | hypothetical protein SORBIDRAFT_02g025010 | | | | |
| | | | | [Sorghum bicolor] | | | | |
| | | BAK06725 | 326504867 | predicted protein [Hordeum vulgare subsp. vulgare] | 0.840647 | Hordeum vulgare subsp. vulgare | 3013 | 4396 |
| | | | | &gt:gi\|326531672\|dbj\|BAJ97840.1\| predicted protein | | | | |
| | | | | [Hordeum vulgare subsp. vulgare] | | | | |
| | | EAZ09138 | 54362548 | hypothetical protein OsI_31408 | 0.82679 | Oryza sativa Indica Group | 3014 | |
| | | | | [Oryza sativa Indica Group] | | | | |
| | | NP_001063206 | 115479224 | Os09g0422000 [Oryza sativa Japonica Group] | 0.82448 | Oryza sativa Japonica Group | 3015 | 4397 |
| | | | | &gt:gi\|50726120\|dbj\|BAD33641.1\| | | | | |
| | | | | putative hydroxycinnamoyl transferase | | | | |
| | | | | [Oryza sativa Japonica Group] | | | | |
| | | | | &gt:gi\|113631439\|dbj\|BAF25120.1\| | | | | |
| | | | | Os09g0422000 [Oryza sativa Japonica Group] | | | | |
| | | | | &gt:gi\|215678844\|dbj\|BAG95281.1\| unnamed | | | | |
| | | | | protein product [Oryza sativa Japonica Group] | | | | |
| | | NP_001147464 | 226494126 | anthranilate N-benzoyltransferase protein 1 | 0.766744 | Zea mays | 3016 | 4398 |
| | | | | [Zea mays] | | | | |
| | | | | &gt:gi\|195611590\|gb\|ACG27625.1\| | | | | |
| | | | | anthranilate N-benzoyltransferase protein 1 | | | | |
| | | | | [Zea mays] | | | | |
| | | ACF87435 | 194706701 | unknown [Zea mays] | 0.764434 | Zea mays | 3017 | 4399 |
| | | XP_002445569 | 242081600 | hypothetical protein SORBIDRAFT_07g021750 | 0.743649 | Sorghum bicolor | 3018 | 4400 |
| | | | | [Sorghum bicolor] | | | | |
| | | | | &gt:gi\|241941919\|gb\|EES15064.1\| | | | | |
| | | | | hypothetical protein SORBIDRAFT_07g021750 | | | | |
| | | | | [Sorghum bicolor] | | | | |
| | | BAJ94002 | 326489856 | predicted protein | 0.741339 | Hordeum vulgare subsp. vulgare | 3019 | 4401 |
| | | | | [Hordeum vulgare subsp. vulgare] | | | | |
| | | BAJ92446 | 326516581 | predicted protein | 0.720554 | Hordeum vulgare subsp. vulgare | 3020 | 4402 |
| | | | | [Hordeum vulgare subsp. vulgare] | | | | |
| | 231-249 | ACG50012 | 195549568 | SnRK2.8 [Zea mays] | 1 | Zea mays | 3021 | 4403 |
| | 342-360 | XP_002444412 | 242079286 | hypothetical protein SORBIDRAFT_07g021570 | 1 | Sorghum bicolor | 3022 | 4404 |
| | | | | [Sorghum bicolor] | | | | |
| | | | | &gt:gi\|241940762\|gb\|EES13907.1\| | | | | |
| | | | | hypothetical protein SORBIDRAFT_07g021570 | | | | |
| | | | | [Sorghum bicolor] | | | | |
| | | NP_001150060 | 226495678 | mitochondrial glycoprotein [Zea mays] | 0.900433 | Zea mays | 3023 | 4405 |
| | | | | &gt:gi\|195636360\|gb\|ACG37648.1\| | | | | |
| | | | | mitochondrial glycoprotein [Zea mays] | | | | |
| | | | | &gt:gi\|224033179\|gb\|ACN35665.1\| unknown | | | | |
| | | | | [Zea mays] | | | | |
| | 98-116 | NP_001183574 | 308081404 | hypothetical protein LOC100502167 [Zea mays] | 1 | Zea mays | 3024 | 4406 |
| | | | | &gt:gi\|238013154\|gb\|ACR37612.1\| unknown | | | | |
| | | | | [Zea mays] | | | | |
| | | NP_001183715 | 308081496 | hypothetical protein LOC100502308 [Zea mays] | 0.931408 | Zea mays | 3025 | 4407 |
| | | | | &gt:gi\|238014104\|gb\|ACR38087.1\| unknown | | | | |
| | | | | [Zea mays] | | | | |
| | | XP_002458491 | 242058690 | hypothetical protein SORBIDRAFT_03g034670 | 0.859206 | Sorghum bicolor | 3026 | 4408 |
| | | | | [Sorghum bicolor] | | | | |
| | | | | &gt:gi\|241930466\|gb\|EES03611.1\| | | | | |
| | | | | hypothetical protein SORBIDRAFT_03g034670 | | | | |
| | | | | [Sorghum bicolor] | | | | |
| | | BAB56055 | 20975281 | WRKY transcription factor-like | 0.707581 | Oryza sativa Japonica Group | 3027 | 4409 |
| | | | | [Oryza sativa Japonica Group] | | | | |
| | | | | &gt:gi\|46394280\|tpg\|DAA05078.1\| | | | | |
| | | | | TPA_inf: WRKY transcription factor 13 | | | | |
| | | | | [Oryza sativa (japonica cultivar-group)] | | | | |
| | | | | &gt:gi\|58042735\|gb\|AAW63711.1\| WRKY13 | | | | |
| | | | | [Oryza sativa Japonica Group] | | | | |
| | | | | &gt:gi\|215695180\|dbj\|BAG90371.1\| unnamed | | | | |
| | | | | protein product [Oryza sativa Japonica Group] | | | | |
| | | EAY75827 | 54362548 | hypothetical protein OsI_03741 | 0.703971 | Oryza sativa Indica Group | 3028 | |
| | | | | [Oryza sativa Indica Group] | | | | |
| | | | | &gt:gi\|132566305\|gb\|ABO34049.1\| | | | | |
| | | | | defense-responsive protein WRKY13 | | | | |
| | | | | [Oryza sativa Indica Group] | | | | |
| | | EAZ13545 | 54398660 | hypothetical protein OsJ_03461 | 0.703971 | Oryza sativa Japonica Group | 3029 | |
| | | | | [Oryza sativa Japonica Group] | | | | |
| | 370-388 | XP_002465546 | 242036302 | hypothetical protein SORBIDRAFT_01g040900 | 1 | Sorghum bicolor | 3030 | 4410 |
| | | | | [Sorghum bicolor] | | | | |
| | | | | &gt:gi\|241919400\|gb\|EER92544.1\| | | | | |
| | | | | hypothetical protein SORBIDRAFT_01g040900 | | | | |
| | | | | [Sorghum bicolor] | | | | |
| | | NP_001149345 | 226504079 | phosphomevalonate kinase [Zea mays] | 0.916016 | Zea mays | 3031 | 4411 |
| | | | | &gt:gi\|195626562\|gb\|ACG35111.1\| | | | | |
| | | | | phosphomevalonate kinase [Zea mays] | | | | |
| | | ACN31689 | 223975002 | unknown [Zea mays] | 0.916016 | Zea mays | 3032 | 4412 |
| | | ACG35008 | 195626355 | phosphomevalonate kinase [Zea mays] | 0.919922 | Zea mays | 3033 | 4413 |
| | | NP_001049577 | 115451952 | Os03g0253100 [Oryza sativa Japonica Group] | 0.865234 | Oryza sativa Japonica Group | 3034 | 4414 |
| | | | | &gt:gi\|108707213\|gb\|ABF95008.1\| | | | | |
| | | | | phosphomevalonate kinase, putative, expressed | | | | |
| | | | | [Oryza sativa Japonica Group] | | | | |
| | | | | &gt:gi\|113548048\|dbj\|BAF11491.1\| Os03g0253100 | | | | |
| | | | | [Oryza sativa Japonica Group] | | | | |
| | | EEE58712 | 54398660 | hypothetical protein OsJ_10169 | 0.865234 | Oryza sativa Japonica Group | 3035 | |
| | | | | [Oryza sativa Japonica Group] | | | | |
| | | EEC74880 | 54362548 | hypothetical protein OsI_10786 | 0.863281 | Oryza sativa Indica Group | 3036 | |
| | | | | [Oryza sativa Indica Group] | | | | |
| | 821-839 | XP_002437161 | 242093341 | hypothetical protein SORBIDRAFT_10g022190 | 1 | Sorghum bicolor | 3037 | 4415 |
| | | | | [Sorghum bicolor] | | | | |
| | | | | &gt:gi\|241915384\|gb\|EER88528.1\| | | | | |
| | | | | hypothetical protein SORBIDRAFT_10g022190 | | | | |
| | | | | [Sorghum bicolor] | | | | |
| | | NP_001105942 | 162463452 | multidrug resistance protein associated 1 | 0.918409 | Zea mays | 3038 | 4416 |
| | | | | [Zea mays] | | | | |
| | | | | &gt:gi\|37694078\|gb\|AAO72315.1\| | | | | |
| | | | | multidrug resistance associated protein 1 | | | | |
| | | | | [Zea mays] | | | | |
| | | | | &gt:gi\|37694080\|gb\|AAO72316.1\| | | | | |
| | | | | multidrug resistance associated protein 1 | | | | |
| | | | | [Zea mays] | | | | |
| | | BAJ96820 | 326521233 | predicted protein | 0.805125 | Hordeum vulgare subsp. vulgare | 3039 | 4417 |
| | | | | [Hordeum vulgare subsp. vulgare] | | | | |
| | 210-228 | NP_001149657 | 226492307 | serine/threonine-protein kinase SAPK8 | 1 | Zea mays | 3040 | 4418 |
| | | | | [Zea mays] | | | | |
| | | | | &gt:gi\|188011171\|gb\|ACD44937.1\| | | | | |
| | | | | osmotic stress/ABA-activated protein kinase | | | | |
| | | | | [Zea mays] | | | | |
| | | | | &gt:gi\|195629240\|gb\|ACG36261.1\| | | | | |
| | | | | serine/threonine-protein kinase SAPK8 | | | | |
| | | | | [Zea mays] | | | | |
| | | | | &gt:gi\|238011080\|gb\|ACR36575.1\| | | | | |
| | | | | unknown [Zea mays] | | | | |
| Predicted | 794-817 | NP_001143962 | 226501495 | hypothetical protein LOC100276777 | 1 | Zea mays | 3041 | 4419 |
| siRNA | | | | [Zea mays] | | | | |
| 59580 | | | | &gt:gi\|195633825\|gb\|ACG36757.1\| | | | | |
| | | | | hypothetical protein [Zea mays] | | | | |
| | | BAJ96330 | 326516675 | predicted protein | 0.78209 | Hordeum vulgare subsp. vulgare | 3042 | 4420 |
| | | | | [Hordeum vulgare subsp. vulgare] | | | | |
| | | NP_001060795 | 115474396 | Os08g0107100 [Oryza sativa Japonica Group] | 0.81791 | Oryza sativa Japonica Group | 3043 | 4421 |
| | | | | &gt:gi\|42408231\|dbj\|BAD09388.1\| unknown | | | | |
| | | | | protein [Oryza sativa Japonica Group] | | | | |
| | | | | &gt:gi\|113622764\|dbj\|BAF22709.1\| Os08g0107100 | | | | |
| | | | | [Oryza sativa Japonica Group] | | | | |
| | | | | &gt:gi\|215694360\|dbj\|BAG89353.1\| unnamed | | | | |
| | | | | protein product [Oryza sativa Japonica Group] | | | | |
| Predicted | 160-178 | XP_002456611 | 242054930 | hypothetical protein SORBIDRAFT_03g039360 | 1 | Sorghum bicolor | 3044 | 4422 |
| siRNA | | | | [Sorghum bicolor] | | | | |
| 59736 | | | | &gt:gi\|241928586\|gb\|EES01731.1\| | | | | |
| | | | | hypothetical protein SORBIDRAFT_03g039360 | | | | |
| | | | | [Sorghum bicolor] | | | | |
| | | NP_001183165 | 308081376 | hypothetical protein LOC100501536 [Zea mays] | 0.984568 | Zea mays | 3045 | 4423 |
| | | | | &gt:gi\|238009750\|gb\|ACR35910.1\| unknown | | | | |
| | | | | [Zea mays] | | | | |
| | | XP_002441219 | 242090772 | hypothetical protein SORBIDRAFT_09g022580 | 0.96142 | Sorghum bicolor | 3046 | 4424 |
| | | | | [Sorghum bicolor] | | | | |
| | | | | &gt:gi\|241946504\|gb\|EES19649.1\| | | | | |
| | | | | hypothetical protein SORBIDRAFT_09g022580 | | | | |
| | | | | [Sorghum bicolor] | | | | |
| | | ACL53191 | 219885632 | unknown [Zea mays] | 0.929012 | Zea mays | 3047 | 4425 |
| | | NP_001068540 | 115486792 | Os11g0703900 [Oryza sativa Japonica Group] | 0.938272 | Oryza sativa Japonica Group | 3048 | 4426 |
| | | | | &gt:gi\|62733235\|gb\|AAX95352.1\| dnaK-type | | | | |
| | | | | molecular chaperone hsp70 - rice (fragment) | | | | |
| | | | | [Oryza sativa Japonica Group] | | | | |
| | | | | &gt:gi\|77552703\|gb\|ABA95500.1\| Heat shock | | | | |
| | | | | cognate 70 kDa protein, putative, expressed | | | | |
| | | | | [Oryza sativa Japonica Group] | | | | |
| | | | | &gt:gi\|113645762\|dbj\|BAF28903.1\| | | | | |
| | | | | Os11g0703900 [Oryza sativa Japonica Group] | | | | |
| | | AAR17080 | 38325814 | heat shock protein 70-3 | 0.935185 | Nicotiana tabacum | 3049 | 4427 |
| | | | | [Nicotiana tabacum] | | | | |
| | | AAB99745 | 2827001 | HSP70 [Triticum aestivum] | 0.936728 | Triticum aestivum | 3050 | 4428 |
| | | BAK00257 | 326519768 | predicted protein | 0.936728 | Hordeum vulgare subsp. vulgare | 3051 | 4429 |
| | | | | [Hordeum vulgare subsp. vulgare] | | | | |
| | 245-263 | XP_002447095 | 242074317 | hypothetical protein SORBIDRAFT_06g028470 | 1 | Sorghum bicolor | 3052 | 4430 |
| | | | | [Sorghum bicolor] | | | | |
| | | | | &gt:gi\|241938278\|gb\|EES11423.1\| | | | | |
| | | | | hypothetical protein SORBIDRAFT_06g028470 | | | | |
| | | | | [Sorghum bicolor] | | | | |
| | | NP_001053867 | 115460533 | Os04g0615100 [Oryza sativa Japonica Group] | 0.819887 | Oryza sativa Japonica Group | 3053 | 4431 |
| | | | | &gt:gi\|38344254\|emb\|CAD41792.2\| | | | | |
| | | | | OSJNBa0008M17.7 [Oryza sativa Japonica Group] | | | | |
| | | | | &gt:gi\|113565438\|dbj\|BAF15781.1\| Os04g0615100 | | | | |
| | | | | [Oryza sativa Japonica Group] | | | | |
| | | | | &gt:gi\|215697586\|dbj\|BAG91580.1\| unnamed | | | | |
| | | | | protein product [Oryza sativa Japonica Group] | | | | |
| | | | | &gt:gi\|218195572\|gb\|EEC77999.1\| hypothetical | | | | |
| | | | | protein OsI_17392 [Oryza sativa Indica Group] | | | | |
| | | | | &gt:gi\|222629547\|gb\|EEE61679.1\| hypothetical | | | | |
| | | | | protein OsJ_16148 [Oryza sativa Japonica Group] | | | | |
| | | NP_001170155 | 293334500 | hypothetical protein LOC100384087 [Zea mays] | 0.714822 | Zea mays | 3054 | 4432 |
| | | | | &gt:gi\|224033871\|gb\|ACN36011.1\| unknown | | | | |
| | | | | [Zea mays] | | | | |
| | | ACR34350 | 238006629 | unknown [Zea mays] | 0.714822 | Zea mays | 3055 | 4433 |
| | 448-466 | ACF84208 | 194700247 | unknown [Zea mays] | 1 | Zea mays | 3056 | 4434 |
| | | | | &gt:gi\|194708648\|gb\|ACF88408.1\| unknown | | | | |
| | | | | [Zea mays] | | | | |
| | | NP_001105049 | 162464284 | D-type cyclin [Zea mays] | 0.98017 | Zea mays | 3057 | 4435 |
| | | | | &gt:gi\|19070613\|gb\|AAL83927.1\|AF351190_1 | | | | |
| | | | | D-type cyclin [Zea mays] | | | | |
| | | ACG35290 | 195626919 | cyclin delta-3 [Zea mays] | 0.963173 | Zea mays | 3058 | 4436 |
| | | ACN27070 | 223945972 | unknown [Zea mays] | 0.750708 | Zea mays | 3059 | 4437 |
| | | ACG25429 | 195607197 | cyclin delta-3 [Zea mays] | 0.747875 | Zea mays | 3060 | 4438 |
| | | XP_002465679 | 242036568 | hypothetical protein SORBIDRAFT_01g043610 | 0.767705 | Sorghum bicolor | 3061 | 4439 |
| | | | | [Sorghum bicolor] | | | | |
| | | | | &gt:gi\|241919533\|gb\|EER92677.1\| | | | | |
| | | | | hypothetical protein SORBIDRAFT_01g043610 | | | | |
| | | | | [Sorghum bicolor] | | | | |
| | | NP_001105863 | 162459778 | cyclin D5,2 [Zea mays] | 0.725212 | Zea mays | 3062 | 4440 |
| | | | | &gt:gi\|61741624\|gb\|AAX54698.1\| | | | | |
| | | | | cyclin D5,3B [Zea mays] | | | | |
| | 167-185 | NP_001055754 | 297604614 | Os05g0460000 [Oryza sativa Japonica Group] | 1 | Oryza sativa Japonica Group | 3063 | 4441 |
| | | | | &gt:gi\|47900318\|gb\|AAT39165.1\| putative hsp70 | | | | |
| | | | | [Oryza sativa Japonica Group] | | | | |
| | | | | &gt:gi\|113579305\|dbj\|BAF17668.1\| Os05g0460000 | | | | |
| | | | | [Oryza sativa Japonica Group] | | | | |
| | | | | &gt:gi\|215769174\|dbj\|BAH01403.1\| | | | | |
| | | | | unnamed protein product | | | | |
| | | | | [Oryza sativa Japonica Group] | | | | |
| | | | | &gt:gi\|222631852\|gb\|EEE63984.1\| hypothetical | | | | |
| | | | | protein OsJ_18811 [Oryza sativa Japonica Group] | | | | |
| | | XP_002311161 | 224098389 | predicted protein [Populus trichocarpa] | 0.94582 | Populus trichocarpa | 3064 | 4442 |
| | | | | &gt:gi\|222850981\|gb\|EEE88528.1\| predicted | | | | |
| | | | | protein [Populus trichocarpa] | | | | |
| | | ACJ11741 | 211906495 | heat shock protein 70 [Gossypium hirsutum] | 0.944272 | Gossypium hirsutum | 3065 | 4443 |
| | | ACD45076 | 188011547 | heat-shock protein 70 [Dactylis glomerata] | 0.948916 | Dactylis glomerata | 3066 | 4444 |
| | 160-178 | NP_001183626 | 308081354 | hypothetical protein LOC100502220 [Zea mays] | 1 | Zea mays | 3067 | 4445 |
| | | | | &gt:gi\|238013512\|gb\|ACR37791.1\| unknown | | | | |
| | | | | [Zea mays] | | | | |
| | | XP_002457205 | 242056118 | hypothetical protein SORBIDRAFT_03g003290 | 0.838791 | Sorghum bicolor | 3068 | 4446 |
| | | | | [Sorghum bicolor] | | | | |
| | | | | &gt:gi\|241929180\|gb\|EES02325.1\| | | | | |
| | | | | hypothetical protein SORBIDRAFT_03g003290 | | | | |
| | | | | [Sorghum bicolor] | | | | |
| | 42-60 | ACG31293 | 195618925 | histone H4 [Zea mays] | 1 | Zea mays | 3069 | 4447 |
| | | | | &gt:gi\|195641748\|gb\|ACG40342.1\| histone | | | | |
| | | | | H4 [Zea mays] | | | | |
| | | ACG31227 | 195618793 | histone H4 [Zea mays] | 0.96875 | Zea mays | 3070 | 4448 |
| | | ACD76815 | 189099146 | histone 4 [Capsella bursa-pastoris] | 0.96875 | Capsella bursa-pastoris | 3071 | 4449 |
| | | 1101277A | | histone H4 | 0.96875 | Triticum aestivum | 3072 | |
| | | P62786 | | RecName: Full = Histone H4 variant TH091 | 0.96875 | Triticum aestivum | 3073 | |
| | | | | &gt:gi\|170747\|gb\|AAA34292.1\| histone H4 | | | | |
| | | | | [Triticum aestivum] | | | | |
| | | ACG31455 | 195619249 | histone H4 [Zea mays] | 0.96875 | Zea mays | 3074 | 4450 |
| | | NP_180441 | 42569420 | histone H4 [Arabidopsis thaliana] | 0.96875 | Arabidopsis thaliana | 3075 | 4451 |
| | | | | &gt:gi\|15231283\|ref\|NP_190179.1\| | | | | |
| | | | | histone H4 [Arabidopsis thaliana] | | | | |
| | | | | &gt:gi\|15232318\|ref\|NP_190941.1\| | | | | |
| | | | | histone H4 [Arabidopsis thaliana] | | | | |
| | | | | &gt:gi\|18390794\|ref\|NP_563793.1\| | | | | |
| | | | | histone H4 [Arabidopsis thaliana] | | | | |
| | | | | &gt:gi\|18390815\|ref\|NP_563797.1\| | | | | |
| | | | | histone H4 [Arabidopsis thaliana] | | | | |
| | | | | &gt:gi\|18424269\|ref\|NP_568911.1\| | | | | |
| | | | | histone H4 [Arabidopsis thaliana] | | | | |
| | | | | &gt:gi\|18424305\|ref\|NP_568918.1\| | | | | |
| | | | | histone H4 [Arabidopsis thaliana] | | | | |
| | | | | &gt:gi\|30680368\|ref\|NP_850939.1\| | | | | |
| | | | | histone H4 [Arabidopsis thaliana] | | | | |
| | | | | &gt:gi\|30692704\|ref\|NP_850660.1\| | | | | |
| | | | | histone H4 [Arabidopsis thaliana] | | | | |
| | | | | &gt:gi\|115447965\|ref\|NP_001047762.1\| | | | | |
| | | | | Os02g0684500 [Oryza sativa Japonica Group] | | | | |
| | | | | &gt:gi\|115450365\|ref\|NP_001048783.1\| | | | | |
| | | | | Os03g0119900 [Oryza sativa Japonica Group] | | | | |
| | | | | &gt:gi\|115460128\|ref\|NP_001053664.1\| | | | | |
| | | | | Os04g0583600 [Oryza sativa Japonica Group] | | | | |
| | | | | &gt:gi\|115464339\|ref\|NP_001055769.1\| | | | | |
| | | | | Os05g0462700 [Oryza sativa Japonica Group] | | | | |
| | | | | &gt:gi\|115464373\|ref\|NP_001055786.1\| | | | | |
| | | | | Os05g0466600 [Oryza sativa Japonica Group] | | | | |
| | | | | &gt:gi\|115479303\|ref\|NP_001063245.1\| | | | | |
| | | | | Os09g0433600 [Oryza sativa Japonica Group] | | | | |
| | | | | &gt:gi\|115480569\|ref\|NP_001063878.1\| | | | | |
| | | | | Os09g0553100 [Oryza sativa Japonica Group] | | | | |
| | | | | &gt:gi\|1115483172\|ref\|NP_001065 179.1\| | | | | |
| | | | | Os10g0539500 [Oryza sativa Japonica Group] | | | | |
| | | | | &gt:gi\|212722314\|ref\|NP_001131585.1\| | | | | |
| | | | | histone H4 [Zea mays] | | | | |
| | | | | &gt:gi\|297597921\|ref\|NP_001044729.2l | | | | |
| | | | | Os01g0835900 [Oryza sativa Japonica Group] | | | | |
| | | | | &gt:gi\|297725773\|ref\|NP_001175250.1\| | | | | |
| | | | | Os07g0549900 [Oryza sativa Japonica Group] | | | | |
| | | | | &gt:gi\|167998046\|ref\|XP_001751729.1\| histone | | | | |
| | | | | H4 [Physcomitrella patens subsp. patens] | | | | |
| | | | | &gt:gi\|168027663\|ref\|XP_001766349.1\| histone | | | | |
| | | | | H4 [Physcomitrella patens subsp. patens] | | | | |
| | | | | &gt:gi\|168031388\|ref\|XP_001768203.1\| histone | | | | |
| | | | | H4 [Physcomitrella patens subsp. patens] | | | | |
| | | | | &gt:gi\|168034381\|ref\|XP_001769691.1\| histone | | | | |
| | | | | H4 [Physcomitrella patens subsp. patens] | | | | |
| | | | | &gt:gi\|168037263\|ref\|XP_001771124.1\| histone | | | | |
| | | | | H4 [Physcomitrella patens subsp. patens] | | | | |
| | | | | &gt:gi\|168042214\|ref\|XP_001773584.1\| histone | | | | |
| | | | | H4 [Physcomitrella patens subsp. patens] | | | | |
| | | | | &gt:gi\|168046645\|ref\|XP_001775783.1\| | | | | |
| | | | | histone H4 [Physcomitrella patens subsp. patens] | | | | |
| | | | | &gt:gi\|168054207\|ref\|XP_001779524.1\| predicted | | | | |
| | | | | protein [Physcomitrella patens subsp. patens] | | | | |
| | | | | &gt:gi\|168055941\|ref\|XP_001779981.1\| histone | | | | |
| | | | | H4 [Physcomitrella patens subsp. patens] | | | | |
| | | | | &gt:gi\|168056875\|ref\|XP_001780443.1\| histone | | | | |
| | | | | H4 [Physcomitrella patens subsp. patens] | | | | |
| | | | | &gt:gi\|168063722\|ref\|XP_001783818.1\| histone | | | | |
| | | | | H4 [Physcomitrella patens subsp. patens] | | | | |
| | | | | &gt:gi\|224097150\|ref\|XP_002310853.1\| | | | | |
| | | | | histone H4 [Populus trichocarpa] | | | | |
| | | | | &gt:gi\|224097156\|ref\|XP_002310855.1\| | | | | |
| | | | | histone H4 [Populus trichocarpa] | | | | |
| | | | | &gt:gi\|224097164\|ref\|XP_002310859.1\| | | | | |
| | | | | histone H4 [Populus trichocarpa] | | | | |
| | | | | &gt:gi\|224098168\|ref\|XP_002311129.1\| | | | | |
| | | | | histone H4 [Populus trichocarpa] | | | | |
| | | | | &gt:gi\|224112905\|ref\|XP_002316326.1\| | | | | |
| | | | | histone H4 [Populus trichocarpa] | | | | |
| | | | | &gt:gi\|224126063\|ref\|XP_002329652.1\| | | | | |
| | | | | histone H4 [Populus trichocarpa] | | | | |
| | | | | &gt:gi\|224133734\|ref\|XP_002327667.1\| | | | | |
| | | | | histone H4 [Populus trichocarpa] | | | | |
| | | | | &gt:gi\|224133742\|ref\|XP_002327669.1\| | | | | |
| | | | | histone H4 [Populus trichocarpa] | | | | |
| | | | | &gt:gi\|224142251\|ref\|XP_002324472.1\| | | | | |
| | | | | histone H4 [Populus trichocarpa] | | | | |
| | | | | &gt:gi\|224142255\|ref\|XP_002324474.1\| | | | | |
| | | | | histone H4 [Populus trichocarpa] | | | | |
| | | | | &gt:gi\|224143736\|ref\|XP_002325056.1\| | | | | |
| | | | | histone H4 [Populus trichocarpa] | | | | |
| | | | | &gt:gi\|224167386\|ref\|XP_002339024.1\| | | | | |
| | | | | histone H4 [Populus trichocarpa] | | | | |
| | | | | &gt:gi\|225435016\|ref\|XP_002284158.1\| | | | | |
| | | | | PREDICTED: hypothetical protein | | | | |
| | | | | isoform 1 [Vitis vinifera] | | | | |
| | | | | &gt:gi\|225435124\|ref\|XP_002284569.1\| | | | | |
| | | | | PREDICTED: hypothetical protein | | | | |
| | | | | isoform 2 [Vitis vinifera] | | | | |
| | | | | &gt:gi\|225435126\|ref\|XP_002284564.1\| | | | | |
| | | | | PREDICTED: hypothetical protein | | | | |
| | | | | isoform 1 [Vitis vinifera] | | | | |
| | | | | &gt:gi\|225440304\|ref\|XP_002262845.1\| | | | | |
| | | | | PREDICTED: hypothetical protein | | | | |
| | | | | isoform 1 [Vitis vinifera] | | | | |
| | | | | &gt:gi\|225448771\|ref\|XP_002281801.1\| | | | | |
| | | | | PREDICTED: hypothetical protein | | | | |
| | | | | isoform 2 [Vitis vinifera] | | | | |
| | | | | &gt:gi\|225448773\|ref\|XP_002281789.1\| | | | | |
| | | | | PREDICTED: hypothetical protein | | | | |
| | | | | isoform 1 [Vitis vinifera] | | | | |
| | | | | &gt:gi\|225449567\|ref\|XP_002283894.1\| | | | | |
| | | | | PREDICTED: hypothetical protein | | | | |
| | | | | [Vitis vinifera] | | | | |
| | | | | &gt:gi\|225449569\|ref\|XP_002283901.1\| | | | | |
| | | | | PREDICTED: hypothetical protein | | | | |
| | | | | isoform 1 [Vitis vinifera] | | | | |
| | | | | &gt:gi\|225449573\|ref\|XP_002283912.1\| | | | | |
| | | | | PREDICTED: hypothetical protein | | | | |
| | | | | isoform 1 [Vitis vinifera] | | | | |
| | | | | &gt:gi\|242035235\|ref\|XP_002465012.1\| | | | | |
| | | | | hypothetical protein SORBIDRAFT_01g030460 | | | | |
| | | | | [Sorghum bicolor] | | | | |
| | | | | &gt:gi\|242042459\|ref\|XP_002468624.1\| | | | | |
| | | | | hypothetical protein SORBIDRAFT_01g049250 | | | | |
| | | | | [Sorghum bicolor] | | | | |
| | | | | &gt:gi\|242044758\|ref\|XP_002460250.1\| | | | | |
| | | | | hypothetical protein SORBIDRAFT_02g025440 | | | | |
| | | | | [Sorghum bicolor] | | | | |
| | | | | &gt:gi\|242050118\|ref\|XP_002462803.1\| | | | | |
| | | | | hypothetical protein SORBIDRAFT_02g032240 | | | | |
| | | | | [Sorghum bicolor] | | | | |
| | | | | &gt:gi\|242051969\|ref\|XP_002455130.1\| | | | | |
| | | | | hypothetical protein SORBIDRAFT_03g004840 | | | | |
| | | | | [Sorghum bicolor] | | | | |
| | | | | &gt:gi\|242051971\|ref\|XP_002455131.1\| | | | | |
| | | | | hypothetical protein SORBIDRAFT_03g004870 | | | | |
| | | | | [Sorghum bicolor] | | | | |
| | | | | &gt:gi\|242054901\|ref\|XP_002456596.1\| | | | | |
| | | | | hypothetical protein SORBIDRAFT_03g039090 | | | | |
| | | | | [Sorghum bicolor] | | | | |
| | | | | &gt:gi\|242056281\|ref\|XP_002457286.1\| | | | | |
| | | | | hypothetical protein SORBIDRAFT_03g004890 | | | | |
| | | | | [Sorghum bicolor] | | | | |
| | | | | &gt:gi\|242062908\|ref\|XP_002452743.1\| | | | | |
| | | | | hypothetical protein SORBIDRAFT_04g031620 | | | | |
| | | | | [Sorghum bicolor] | | | | |
| | | | | &gt:gi\|242076918\|ref\|XP_002448395.1\| | | | | |
| | | | | hypothetical protein SORBIDRAFT_06g026490 | | | | |
| | | | | [Sorghum bicolor] | | | | |
| | | | | &gt:gi\|242088199\|ref\|XP_002439932.1\| | | | | |
| | | | | hypothetical protein SORBIDRAFT_09g022920 | | | | |
| | | | | [Sorghum bicolor] | | | | |
| | | | | &gt:gi\|255537239\|ref\|XP_002509686.1\| | | | | |
| | | | | histone h4, putative [Ricinus communis] | | | | |
| | | | | &gt:gi\|255555809\|ref\|XP_002518940.1\| | | | | |
| | | | | histone h4, putative [Ricinus communis] | | | | |
| | | | | &gt:gi\|255568195\|ref\|XP_002525073.1\| | | | | |
| | | | | histone h4, putative [Ricinus communis] | | | | |
| | | | | &gt:gi\|255581703\|ref\|XP_002531654.1\| | | | | |
| | | | | histone h4, putative [Ricinus communis] | | | | |
| | | | | &gt:gi\|255581707\|ref\|XP_002531656.1\| | | | | |
| | | | | histone h4, putative [Ricinus communis] | | | | |
| | | | | &gt:gi\|255584136\|ref\|XP_002532808.1\| | | | | |
| | | | | histone h4, putative [Ricinus communis] | | | | |
| | | | | &gt:gi\|297793525\|ref\|XP_002864647.1\| | | | | |
| | | | | hypothetical protein ARALYDRAFT_496101 | | | | |
| | | | | [Arabidopsis lyrata subsp. lyrata] | | | | |
| | | | | &gt:gi\|297815748\|ref\|XP_002875757.1\| | | | | |
| | | | | hypothetical protein ARALYDRAFT_484971 | | | | |
| | | | | [Arabidopsis lyrata subsp. lyrata] | | | | |
| | | | | &gt:gi\|297819196\|ref\|XP_002877481.1\| | | | | |
| | | | | hypothetical protein ARALYDRAFT_485010 | | | | |
| | | | | [Arabidopsis lyrata subsp. lyrata] | | | | |
| | | | | &gt:gi\|297820110\|ref\|XP_002877938.1\| | | | | |
| | | | | hypothetical protein ARALYDRAFT_485763 | | | | |
| | | | | [Arabidopsis lyrata subsp. lyrata] | | | | |
| | | | | &gt:gi\|297826247\|ref\|XP_002881006.1\| | | | | |
| | | | | hypothetical protein ARALYDRAFT_481787 | | | | |
| | | | | [Arabidopsis lyrata subsp. lyrata] | | | | |
| | | | | &gt:gi\|28202123\|sp\|P59259.2\|H4_ARATH | | | | |
| | | | | RecName: Full = Histone H4 | | | | |
| | | | | &gt:gi\|51315699\|sp\|Q6LAF3.3\|H4_FLATR | | | | |
| | | | | RecName: Full = Histone H4 | | | | |
| | | | | &gt:gi\|51315702\|sp\|Q6PMI5.3\|H4_CHEMJ | | | | |
| | | | | RecName: Full = Histone H4 | | | | |
| | | | | &gt:gi\|51315711\|sp\|Q6WZ83.3\|H4_EUCGL | | | | |
| | | | | RecName: Full = Histone H4 | | | | |
| | | | | &gt:gi\|51315719\|sp\|Q76H85.3\|H4_SILLA | | | | |
| | | | | RecName: Full = Histone H4 | | | | |
| | | | | &gt:gi\|51317313\|sp\|P62788.2\|H4_PEA | | | | |
| | | | | RecName: Full = Histone H4 | | | | |
| | | | | &gt:gi\|51317325\|sp\|P62787.2\|H4_MAIZE | | | | |
| | | | | RecName: Full = Histone H4 | | | | |
| | | | | &gt:gi\|51317341\|sp\|P62887.2\|H4_LOLTE | | | | |
| | | | | RecName: Full = Histone H4 | | | | |
| | | | | &gt:gi\|78100002\|sp\|P62785.2\|H41_WHEAT | | | | |
| | | | | RecName: Full = Histone H4 variant TH011 | | | | |
| | | | | &gt:gi\|302425021\|sp\|P0CG89.1\|H4_SOYBN | | | | |
| | | | | RecName: Full = Histone H4 | | | | |
| | | | | &gt:gi\|8439886\|gb\|AAF75072.1\|AC007583_8 | | | | |
| | | | | Identical to histone H4 from | | | | |
| | | | | Arabidopsis thaliana gi\|S06904 | | | | |
| | | | | &gt:gi\|8439903\|gb\|AAF75089.1\|AC007583_25 | | | | |
| | | | | Identical to histone H4 from | | | | |
| | | | | Arabidopsis thaliana gi\|S06904 | | | | |
| | | | | &gt:gi\|11762277\|gb\|AAG40410.1\|AF325058_1 | | | | |
| | | | | AT5g59690 [Arabidopsis thaliana] | | | | |
| | | | | &gt:gi\|12039318\|gb\|AAG46106.1\|AC073166_4 | | | | |
| | | | | histone H4 [Oryza sativa Japonica Group] | | | | |
| | | | | &gt:gi\|12248031\|gb\|AAG50107.1\|AF334729_1 | | | | |
| | | | | putative histone H4 protein [Arabidopsis thaliana] | | | | |
| | | | | &gt:gi\|21795\|emb\|CAA24924.1\| | | | | |
| | | | | unnamed protein product [Triticum aestivum] | | | | |
| | | | | &gt:gi\|166740\|gb\|AAA32810.1\| | | | | |
| | | | | histone H4 [Arabidopsis thaliana] | | | | |
| | | | | &gt:gi\|166742\|gb\|AAA32811.1\| | | | | |
| | | | | histone H4 [Arabidopsis thaliana] | | | | |
| | | | | &gt:gi\|168499\|gb\|AAA33474.1\| | | | | |
| | | | | histone H4 (H4C13) [Zea mays] | | | | |
| | | | | &gt:gi\|168501\|gb\|AAA33475.1\| | | | | |
| | | | | histone H4 [Zea mays] | | | | |
| | | | | &gt:gi\|168503\|gb\|AAA33476.1\| | | | | |
| | | | | histone H4 [Zea mays] | | | | |
| | | | | &gt:gi\|498898\|gb\|AAA86948.1\| | | | | |
| | | | | histone H4 homolog [Pisum sativum] | | | | |
| | | | | &gt:gi\|1806285\|emb\|CAB01914.1\| | | | | |
| | | | | histone H4 homologue [Sesbania rostrata] | | | | |
| | | | | &gt:gi\|3927823\|gb\|AAC79580.1\| | | | | |
| | | | | histone H4 [Arabidopsis thaliana] | | | | |
| | | | | &gt:gi\|6009915\|dbj\|BAA85120.1\| | | | | |
| | | | | histone H4-like protein [Solanum melongena] | | | | |
| | | | | &gt:gi\|6522611\|emb\|CAB62023.1\| | | | | |
| | | | | histone H4-like protein [Arabidopsis thaliana] | | | | |
| | | | | &gt:gi\|7339494\|emb\|CAB82817.1\| | | | | |
| | | | | Histone H4-like protein [Arabidopsis thaliana] | | | | |
| | | | | &gt:gi\|7629993\|emb\|CAB88335.1\| | | | | |
| | | | | histone H4-like protein [Arabidopsis thaliana] | | | | |
| | | | | &gt:gi\|9757918\|dbj\|BAB08365.1\| | | | | |
| | | | | histone H4 [Arabidopsis thaliana] | | | | |
| | | | | &gt:gi\|9758835\|dbj\|BAB09507.1\| | | | | |
| | | | | histone H4 [Arabidopsis thaliana] | | | | |
| | | | | &gt:gi\|13277212\|emb\|CAC34411.1\| | | | | |
| | | | | histone H4 [Flaveria trinervia] | | | | |
| | | | | &gt:gi\|16209693\|gb\|AAL14404.1\| | | | | |
| | | | | AT5g59690/mth12_90 [Arabidopsis thaliana] | | | | |
| | | | | &gt:gi\|17065282\|gb\|AAL32795.1\| | | | | |
| | | | | histone H4-like protein [Arabidopsis thaliana] | | | | |
| | | | | &gt:gi\|17380766\|gb\|AAL36213.1\| putative | | | | |
| | | | | histone H4 protein [Arabidopsis thaliana] | | | | |
| | | | | &gt:gi\|20160804\|dbj\|BAB89744.1\| | | | | |
| | | | | histone H4 [Oryza sativa Japonica Group] | | | | |
| | | | | &gt:gi\|20198175\|gb\|AAM15445.1\| | | | | |
| | | | | histone H4 [Arabidopsis thaliana] | | | | |
| | | | | &gt:gi\|20260010\|gb\|AAM13352.1\| | | | | |
| | | | | histone H4-like protein [Arabidopsis thaliana] | | | | |
| | | | | &gt:gi\|20466418\|gb\|AAM20526.1\| | | | | |
| | | | | histone H4-like protein [Arabidopsis thaliana] | | | | |
| | | | | &gt:gi\|21537385\|gb\|AAM61726.1\| | | | | |
| | | | | histone H4-like protein [Arabidopsis thaliana] | | | | |
| | | | | &gt:gi\|21553628\|gb\|AAM62721.1\| | | | | |
| | | | | histone H4-like protein [Arabidopsis thaliana] | | | | |
| | | | | &gt:gi\|21554094\|gb\|AAM63175.1\| | | | | |
| | | | | histone H4-like protein [Arabidopsis thaliana] | | | | |
| | | | | &gt:gi\|21555353\|gb\|AAM63839.1\| | | | | |
| | | | | histone H4-like protein [Arabidopsis thaliana] | | | | |
| | | | | &gt:gi\|21592313\|gb\|AAM64264.1\| | | | | |
| | | | | histone H4-like protein [Arabidopsis thaliana] | | | | |
| | | | | &gt:gi\|21592673\|gb\|AAM64622.1\| | | | | |
| | | | | histone H4-like protein [Arabidopsis thaliana] | | | | |
| | | | | &gt:gi\|21592795\|gb\|AAM64744.1\| | | | | |
| | | | | histone H4-like protein [Arabidopsis thaliana] | | | | |
| | | | | &gt:gi\|21700843\|gb\|AAM70545.1\| | | | | |
| | | | | AT5g59690/mth12_90 [Arabidopsis thaliana] | | | | |
| | | | | &gt:gi\|22136354\|gb\|AAM91255.1\| | | | | |
| | | | | histone H4-like protein | | | | |
| | | | | [Arabidopsis thaliana] | | | | |
| | | | | &gt:gi\|22165124\|gb\|AAM93740.1\| | | | | |
| | | | | histone H4 [Oryza sativa Japonica Group] | | | | |
| | | | | &gt:gi\|23296862\|gb\|AAN13189.1\| | | | | |
| | | | | putative histone H4 | | | | |
| | | | | protein [Arabidopsis thaliana] | | | | |
| | | | | &gt:gi\|27452909\|gb\|AAO15293.1\| | | | | |
| | | | | Unknown protein [Oryza sativa Japonica Group] | | | | |
| | | | | &gt:gi\|28208264\|dbj\|BAC56852.1\| | | | | |
| | | | | histone H4 [Silene latifolia] | | | | |
| | | | | &gt:gi\|28393088\|gb\|AAO41978.1\| | | | | |
| | | | | putative histone H4 protein | | | | |
| | | | | [Arabidopsis thaliana] | | | | |
| | | | | &gt:gi\|28466803\|gb\|AAO44010.1\| | | | | |
| | | | | At1g07820 [Arabidopsis thaliana] | | | | |
| | | | | &gt:gi\|28564804\|dbj\|BAC57734.1\| | | | | |
| | | | | histone H4 [Oryza sativa Japonica Group] | | | | |
| | | | | &gt:gi\|28827318\|gb\|AAO50503.1\| | | | | |
| | | | | putative histone H4 | | | | |
| | | | | protein [Arabidopsis thaliana] | | | | |
| | | | | &gt:gi\|30575604\|gb\|AAP33088.1\| | | | | |
| | | | | histone H4 [Eucalyptus globulus] | | | | |
| | | | | &gt:gi\|31433309\|gb\|AAP54838.1\| | | | | |
| | | | | Histone H4, putative, expressed | | | | |
| | | | | [Oryza sativa Japonica Group] | | | | |
| | | | | &gt:gi\|38346810\|emb\|CAD41377.2\| | | | | |
| | | | | OSJNBa0088A01.17 [Oryza sativa Japonica Group] | | | | |
| | | | | &gt:gi\|41052706\|dbj\|BAD07563.1\| | | | | |
| | | | | histone H4 [Oryza sativa Japonica Group] | | | | |
| | | | | &gt:gi\|46811262\|gb\|AAT01924.1\| | | | | |
| | | | | histone H4 [Chelidonium majus] | | | | |
| | | | | &gt:gi\|47900360\|gb\|AAT39190.1\| | | | | |
| | | | | putative histone H4 [Oryza sativa Japonica Group] | | | | |
| | | | | &gt:gi\|49328063\|gb\|AAT58763.1\| | | | | |
| | | | | histone H4 [Oryza sativa Japonica Group] | | | | |
| | | | | &gt:gi\|49328086\|gb\|AAT58785.1\| | | | | |
| | | | | histone H4 [Oryza sativa Japonica Group] | | | | |
| | | | | &gt:gi\|50251938\|dbj\|BAD27874.1\| | | | | |
| | | | | histone H4 [Oryza sativa Japonica Group] | | | | |
| | | | | &gt:gi\|50726031\|dbj\|BAD33556.1\| | | | | |
| | | | | histone H4 [Oryza sativa Japonica Group] | | | | |
| | | | | &gt:gi\|51969168\|dbj\|BAD43276.1\| | | | | |
| | | | | histone H4 [Arabidopsis thaliana] | | | | |
| | | | | &gt:gi\|51969828\|dbj\|BAD43606.1\| | | | | |
| | | | | histone H4 [Arabidopsis thaliana] | | | | |
| | | | | &gt:gi\|51970436\|dbj\|BAD43910.1\| | | | | |
| | | | | histone H4 [Arabidopsis thaliana] | | | | |
| | | | | &gt:gi\|53749311\|gb\|AAU90170.1\| | | | | |
| | | | | histone H4 [Oryza sativa Japonica Group] | | | | |
| | | | | &gt:gi\|56798269\|dbj\|BAD82897.1\| | | | | |
| | | | | histone H4 [Fragaria × ananassa] | | | | |
| | | | | &gt:gi\|62642127\|gb\|AAX92702.1\| | | | | |
| | | | | histone 4 [Picea abies] | | | | |
| | | | | &gt:gi\|87138105\|gb\|ABD28289.1\| | | | | |
| | | | | histone H4-like protein [Glycine max] | | | | |
| | | | | &gt:gi\|88010997\|gb\|ABD38885.1\| | | | | |
| | | | | At3g45930 [Arabidopsis thaliana] | | | | |
| | | | | &gt:gi\|92885100\|gb\|ABE87620.1\| | | | | |
| | | | | Histone core [Medicago truncatula] | | | | |
| | | | | &gt:gi\|108705887\|gb\|ABF93682.1\| | | | | |
| | | | | Histone H4, putative, expressed | | | | |
| | | | | [Oryza sativa Japonica Group] | | | | |
| | | | | &gt:gi\|110738359\|dbj\|BAF01106.1 | | | | |
| | | | | Histone H4 like protein [Arabidopsis thaliana] | | | | |
| | | | | &gt:gi\|110742734\|dbj\|BAF00179.1\| | | | | |
| | | | | histone H4 [Arabidopsis thaliana] | | | | |
| | | | | &gt:gi\|113537293\|dbj\|BAF09676.1\| | | | | |
| | | | | Os02g0684500 [Oryza sativa Japonica Group] | | | | |
| | | | | &gt:gi\|113547254\|dbj\|BAF10697.1\| | | | | |
| | | | | Os03g0119900 [Oryza sativa Japonica Group] | | | | |
| | | | | &gt:gi\|113565235\|dbj\|BAF15578.1\| | | | | |
| | | | | Os04g0583600 [Oryza sativa Japonica Group] | | | | |
| | | | | &gt:gi\|113579320\|dbj\|BAF17683.1\| | | | | |
| | | | | Os05g0462700 [Oryza sativa Japonica Group] | | | | |
| | | | | &gt:gi\|113579337\|dbj\|BAF17700.1\| | | | | |
| | | | | Os05g0466600 [Oryza sativa Japonica Group] | | | | |
| | | | | &gt:gi\|113631478\|dbj\|BAF25159.1\| | | | | |
| | | | | Os09g0433600 [Oryza sativa Japonica Group] | | | | |
| | | | | &gt:gi\|113632111\|dbj\|BAF25792.1\| | | | | |
| | | | | Os09g0553100 [Oryza sativa Japonica Group] | | | | |
| | | | | &gt:gi\|113639788\|dbj\|BAF27093.1\| | | | | |
| | | | | Os10g0539500 [Oryza sativa Japonica Group] | | | | |
| | | | | &gt:gi\|116778467\|gb\|ABK20879.1\| | | | | |
| | | | | unknown [Picea sitchensis] | | | | |
| | | | | &gt:gi\|116782704\|gb\|ABK22619.1\| | | | | |
| | | | | unknown [Picea sitchensis] | | | | |
| | | | | &gt:gi\|116788052\|gb\|ABK24738.1\| | | | | |
| | | | | unknown [Picea sitchensis] | | | | |
| | | | | &gt:gi\|116793524\|gb\|ABK26777.1\| | | | | |
| | | | | unknown [Picea sitchensis] | | | | |
| | | | | &gt:gi\|118482735\|gb\|ABK93286.1\| | | | | |
| | | | | unknown [Populus trichocarpa] | | | | |
| | | | | &gt:gi\|118484754\|gb\|ABK94246.1\| | | | | |
| | | | | unknown [Populus trichocarpa] | | | | |
| | | | | &gt:gi\|118485565\|gb\|ABK94634.1\| | | | | |
| | | | | unknown [Populus trichocarpa] | | | | |
| | | | | &gt:gi\|124360937\|gb\|ABN08909.1\| | | | | |
| | | | | Histone core [Medicago truncatula] | | | | |
| | | | | &gt:gi\|125528296\|gb\|EAY76410.1\| | | | | |
| | | | | hypothetical protein OsI_04340 | | | | |
| | | | | [Oryza sativa Indica Group] | | | | |
| | | | | &gt:gi\|125532798\|gb\|EAY79363.1\| | | | | |
| | | | | hypothetical protein OsI_34491 | | | | |
| | | | | [Oryza sativa Indica Group] | | | | |
| | | | | &gt:gi\|125540704\|gb\|EAY87099.1\| | | | | |
| | | | | hypothetical protein OsI_08497 | | | | |
| | | | | [Oryza sativa Indica Group] | | | | |
| | | | | &gt:gi\|125542165\|gb\|EAY88304.1\| | | | | |
| | | | | hypothetical protein OsI_09762 | | | | |
| | | | | [Oryza sativa Indica Group] | | | | |
| | | | | &gt:gi\|125549476\|gb\|EAY95298.1\| | | | | |
| | | | | hypothetical protein OsI_17123 | | | | |
| | | | | [Oryza sativa Indica Group] | | | | |
| | | | | &gt:gi\|125552628\|gb\|EAY98337.1\| | | | | |
| | | | | hypothetical protein OsI_20247 | | | | |
| | | | | [Oryza sativa Indica Group] | | | | |
| | | | | &gt:gi\|125552649\|gb\|EAY98358.1\| | | | | |
| | | | | hypothetical protein OsI_20269 | | | | |
| | | | | [Oryza sativa Indica Group] | | | | |
| | | | | &gt:gi\|125558734\|gb\|EAZ04270.1\| | | | | |
| | | | | hypothetical protein OsI_26413 | | | | |
| | | | | [Oryza sativa Indica Group] | | | | |
| | | | | &gt:gi\|125563829\|gb\|EAZ09209.1\| | | | | |
| | | | | hypothetical protein OsI_31484 | | | | |
| | | | | [Oryza sativa Indica Group] | | | | |
| | | | | &gt:gi\|125564638\|gb\|EAZ10018.1\| | | | | |
| | | | | hypothetical protein OsI_32321 | | | | |
| | | | | [Oryza sativa Indica Group] | | | | |
| | | | | &gt:gi\|125572554\|gb\|EAZ14069.1\| | | | | |
| | | | | hypothetical protein OsJ_03994 | | | | |
| | | | | [Oryza sativa Japonica Group] | | | | |
| | | | | &gt:gi\|125575549\|gb\|EAZ16833.1\| | | | | |
| | | | | hypothetical protein OsJ_32304 | | | | |
| | | | | [Oryza sativa Japonica Group] | | | | |
| | | | | &gt:gi\|125583277\|gb\|EAZ24208.1\| | | | | |
| | | | | hypothetical protein OsJ_07955 | | | | |
| | | | | [Oryza sativa Japonica Group] | | | | |
| | | | | &gt:gi\|125584717\|gb\|EAZ25381.1\| | | | | |
| | | | | hypothetical protein OsJ_09199 | | | | |
| | | | | [Oryza sativa Japonica Group] | | | | |
| | | | | &gt:gi\|125591413\|gb\|EAZ31763.1\| | | | | |
| | | | | hypothetical protein OsJ_15915 | | | | |
| | | | | [Oryza sativa Japonica Group] | | | | |
| | | | | &gt:gi\|125600645\|gb\|EAZ40221.1\| | | | | |
| | | | | hypothetical protein OsJ_24666 | | | | |
| | | | | [Oryza sativa Japonica Group] | | | | |
| | | | | &gt:gi\|125606566\|gb\|EAZ45602.1\| | | | | |
| | | | | hypothetical protein OsJ_30268 | | | | |
| | | | | [Oryza sativa Japonica Group] | | | | |
| | | | | &gt:gi\|146403794\|gb\|ABQ32303.1\| | | | | |
| | | | | putative histone H4-like protein | | | | |
| | | | | [Artemisia annua] | | | | |
| | | | | &gt:gi\|147800359\|emb\|CAN64268.1\| | | | | |
| | | | | hypothetical protein VITISV_036365 | | | | |
| | | | | [Vitis vinifera] | | | | |
| | | | | &gt:gi\|147826823\|emb\|CAN59705.1\| | | | | |
| | | | | hypothetical protein VITISV_010247 | | | | |
| | | | | [Vitis vinifera] | | | | |
| | | | | &gt:gi\|147826824\|emb\|CAN59706.1\| | | | | |
| | | | | hypothetical protein VITISV_010248 | | | | |
| | | | | [Vitis vinifera] | | | | |
| | | | | &gt:gi\|147839844\|emb\|CAN68239.1\| | | | | |
| | | | | hypothetical protein VITISV_006985 | | | | |
| | | | | [Vitis vinifera] | | | | |
| | | | | &gt:gi\|147842470\|emb\|CAN63143.1\| | | | | |
| | | | | hypothetical protein VITISV_034577 | | | | |
| | | | | [Vitis vinifera] | | | | |
| | | | | &gt:gi\|147855175\|emb\|CAN79580.1\| | | | | |
| | | | | hypothetical protein VITISV_002271 | | | | |
| | | | | [Vitis vinifera] | | | | |
| | | | | &gt:gi\|147855176\|emb\|CAN79581.1\| | | | | |
| | | | | hypothetical protein VITISV_002272 | | | | |
| | | | | [Vitis vinifera] | | | | |
| | | | | &gt:gi\|147855413\|emb\|CAN79612.1\| | | | | |
| | | | | hypothetical protein VITISV_035467 | | | | |
| | | | | [Vitis vinifera] | | | | |
| | | | | &gt:gi\|147858185\|emb\|CAN79680.1\| | | | | |
| | | | | hypothetical protein VITISV_034640 | | | | |
| | | | | [Vitis vinifera] | | | | |
| | | | | &gt:gi\|147859377\|emb\|CAN83554.1\| | | | | |
| | | | | hypothetical protein VITISV_030356 | | | | |
| | | | | [Vitis vinifera] | | | | |
| | | | | &gt:gi\|158828217\|gb\|ABW81095.1\| | | | | |
| | | | | H4his18 [Cleome spinosa] | | | | |
| | | | | &gt:gi\|162664647\|gb\|EDQ51358.1\| histone | | | | |
| | | | | H4 [Physcomitrella patens subsp. patens] | | | | |
| | | | | &gt:gi\|162668119\|gb\|EDQ54733.1\| histone | | | | |
| | | | | H4 [Physcomitrella patens subsp. patens] | | | | |
| | | | | &gt:gi\|162668586\|gb\|EDQ55190.1\| histone | | | | |
| | | | | H4 [Physcomitrella patens subsp. patens] | | | | |
| | | | | &gt:gi\|162669106\|gb\|EDQ55700.1\| predicted protein | | | | |
| | | | | [Physcomitrella patens subsp. patens] | | | | |
| | | | | &gt:gi\|162672790\|gb\|EDQ59322.1\| histone | | | | |
| | | | | H4 [Physcomitrella patens subsp. patens] | | | | |
| | | | | &gt:gi\|162675123\|gb\|EDQ61622.1\| histone | | | | |
| | | | | H4 [Physcomitrella patens subsp. patens] | | | | |
| | | | | &gt:gi\|162677657\|gb\|EDQ64125.1\| histone | | | | |
| | | | | H4 [Physcomitrella patens subsp. patens] | | | | |
| | | | | &gt:gi\|162679040\|gb\|EDQ65492.1\| histone | | | | |
| | | | | H4 [Physcomitrella patens subsp. patens] | | | | |
| | | | | &gt:gi\|162680641\|gb\|EDQ67076.1\| histone | | | | |
| | | | | H4 [Physcomitrella patens subsp. patens] | | | | |
| | | | | &gt:gi\|162682563\|gb\|EDQ68981.1\| histone | | | | |
| | | | | H4 [Physcomitrella patens subsp. patens] | | | | |
| | | | | &gt:gi\|162696827\|gb\|EDQ83164.1\| histone | | | | |
| | | | | H4 [Physcomitrella patens subsp. patens] | | | | |
| | | | | &gt:gi\|194691936\|gb\|ACF80052.1\| | | | | |
| | | | | unknown [Zea mays] | | | | |
| | | | | &gt:gi\|194693488\|gb\|ACF80828.1\| | | | | |
| | | | | unknown [Zea mays] | | | | |
| | | | | &gt:gi\|194696282\|gb\|ACF82225.1\| | | | | |
| | | | | unknown [Zea mays] | | | | |
| | | | | &gt:gi\|194696408\|gb\|ACF82288.1\| | | | | |
| | | | | unknown [Zea mays] | | | | |
| | | | | &gt:gi\|194698290\|gb\|ACF83229.1\| | | | | |
| | | | | unknown [Zea mays] | | | | |
| | | | | &gt:gi\|194698982\|gb\|ACF83575.1\| | | | | |
| | | | | unknown [Zea mays] | | | | |
| | | | | &gt:gi\|194699362\|gb\|ACF83765.1\| | | | | |
| | | | | unknown [Zea mays] | | | | |
| | | | | &gt:gi\|194700348\|gb\|ACF84258.1\| | | | | |
| | | | | unknown [Zea mays] | | | | |
| | | | | &gt:gi\|194704392\|gb\|ACF86280.1\| | | | | |
| | | | | unknown [Zea mays] | | | | |
| | | | | &gt:gi\|194706260\|gb\|ACF87214.1\| | | | | |
| | | | | unknown [Zea mays] | | | | |
| | | | | &gt:gi\|194708346\|gb\|ACF88257.1\| | | | | |
| | | | | unknown [Zea mays] | | | | |
| | | | | &gt:gi\|195605566\|gb\|ACG24613.1\| | | | | |
| | | | | histone H4 [Zea mays] | | | | |
| | | | | &gt:gi\|195605632\|gb\|ACG24646.1\| | | | | |
| | | | | histone H4 [Zea mays] | | | | |
| | | | | &gt:gi\|195605640\|gb\|ACG24650.1\| | | | | |
| | | | | histone H4 [Zea mays] | | | | |
| | | | | &gt:gi\|195605982\|gb\|ACG24821.1\| | | | | |
| | | | | histone H4 [Zea mays] | | | | |
| | | | | &gt:gi\|195606488\|gb\|ACG25074.1\| | | | | |
| | | | | histone H4 [Zea mays] | | | | |
| | | | | &gt:gi\|195606652\|gb\|ACG25 156.1\| | | | | |
| | | | | histone H4 [Zea mays] | | | | |
| | | | | &gt:gi\|195607014\|gb\|ACG25337.1\| | | | | |
| | | | | histone H4 [Zea mays] | | | | |
| | | | | &gt:gi\|195607340\|gb\|ACG25500.1\| | | | | |
| | | | | histone H4 [Zea mays] | | | | |
| | | | | &gt:gi\|195617184\|gb\|ACG30422.1\| | | | | |
| | | | | histone H4 [Zea mays] | | | | |
| | | | | &gt:gi\|195617244\|gb\|ACG30452.1\| | | | | |
| | | | | histone H4 [Zea mays] | | | | |
| | | | | &gt:gi\|195617708\|gb\|ACG30684.1\| | | | | |
| | | | | histone H4 [Zea mays] | | | | |
| | | | | &gt:gi\|195617830\|gb\|ACG30745.1\| | | | | |
| | | | | histone H4 [Zea mays] | | | | |
| | | | | &gt:gi\|195617840\|gb\|ACG30750.1\| | | | | |
| | | | | histone H4 [Zea mays] | | | | |
| | | | | &gt:gi\|195617842\|gb\|ACG3075 1.1\| | | | | |
| | | | | histone H4 [Zea mays] | | | | |
| | | | | &gt:gi\|195617880\|gb\|ACG30770.1\| | | | | |
| | | | | histone H4 [Zea mays] | | | | |
| | | | | &gt:gi\|195618008\|gb\|ACG30834.1\| | | | | |
| | | | | histone H4 [Zea mays] | | | | |
| | | | | &gt:gi\|195618012\|gb\|ACG30836.1\| | | | | |
| | | | | histone H4 [Zea mays] | | | | |
| | | | | &gt:gi\|195618076\|gb\|ACG30868.1\| | | | | |
| | | | | histone H4 [Zea mays] | | | | |
| | | | | &gt:gi\|195618078\|gb\|ACG30869.1\| | | | | |
| | | | | histone H4 [Zea mays] | | | | |
| | | | | &gt:gi\|195618086\|gb\|ACG30873.1\| | | | | |
| | | | | histone H4 [Zea mays] | | | | |
| | | | | &gt:gi\|195618174\|gb\|ACG30917.1\| | | | | |
| | | | | histone H4 [Zea mays] | | | | |
| | | | | &gt:gi\|195618332\|gb\|ACG30996.1\| | | | | |
| | | | | histone H4 [Zea mays] | | | | |
| | | | | &gt:gi\|195618430\|gb\|ACG31045.1\| | | | | |
| | | | | histone H4 [Zea mays] | | | | |
| | | | | &gt:gi\|195618454\|gb\|ACG31057.1\| | | | | |
| | | | | histone H4 [Zea mays] | | | | |
| | | | | &gt:gi\|195618798\|gb\|ACG31229.1\| | | | | |
| | | | | histone H4 [Zea mays] | | | | |
| | | | | &gt:gi\|195618800\|gb\|ACG31230.1\| | | | | |
| | | | | histone H4 [Zea mays] | | | | |
| | | | | &gt:gi\|195618808\|gb\|ACG31234.1\| | | | | |
| | | | | histone H4 [Zea mays] | | | | |
| | | | | &gt:gi\|195618940\|gb\|ACG31300.1\| | | | | |
| | | | | histone H4 [Zea mays] | | | | |
| | | | | &gt:gi\|195618970\|gb\|ACG31315.1\| | | | | |
| | | | | histone H4 [Zea mays] | | | | |
| | | | | &gt:gi\|195620178\|gb\|ACG31919.1\| | | | | |
| | | | | histone H4 [Zea mays] | | | | |
| | | | | &gt:gi\|195621558\|gb\|ACG32609.1\| | | | | |
| | | | | histone H4 [Zea mays] | | | | |
| | | | | &gt:gi\|195623194\|gb\|ACG33427.1\| | | | | |
| | | | | histone H4 [Zea mays] | | | | |
| | | | | &gt:gi\|195625166\|gb\|ACG34413.1\| | | | | |
| | | | | histone H4 [Zea mays] | | | | |
| | | | | &gt:gi\|195626072\|gb\|ACG34866.1\| | | | | |
| | | | | histone H4 [Zea mays] | | | | |
| | | | | &gt:gi\|195628242\|gb\|ACG35951.1\| | | | | |
| | | | | histone H4 [Zea mays] | | | | |
| | | | | &gt:gi\|195628292\|gb\|ACG35976.1\| | | | | |
| | | | | histone H4 [Zea mays] | | | | |
| | | | | &gt:gi\|195628370\|gb\|ACG36015.1\| | | | | |
| | | | | histone H4 [Zea mays] | | | | |
| | | | | &gt:gi\|195629326\|gb\|ACG36304.1\| | | | | |
| | | | | histone H4 [Zea mays] | | | | |
| | | | | &gt:gi\|195630263\|gb\|ACG36622.1\| | | | | |
| | | | | histone H4 [Zea mays] | | | | |
| | | | | &gt:gi\|195635063\|gb\|ACG37000.1\| | | | | |
| | | | | histone H4 [Zea mays] | | | | |
| | | | | &gt:gi\|195635563\|gb\|ACG37250.1\| | | | | |
| | | | | histone H4 [Zea mays] | | | | |
| | | | | &gt:gi\|195636274\|gb\|ACG37605.1\| | | | | |
| | | | | histone H4 [Zea mays] | | | | |
| | | | | &gt:gi\|195636714\|gb\|ACG37825.1\| | | | | |
| | | | | histone H4 [Zea mays] | | | | |
| | | | | &gt:gi\|195638688\|gb\|ACG388 12.1\| | | | | |
| | | | | histone H4 [Zea mays] | | | | |
| | | | | &gt:gi\|195639506\|gb\|ACG39221.1\| | | | | |
| | | | | histone H4 [Zea mays] | | | | |
| | | | | &gt:gi\|195658023\|gb\|ACG48479.1\| | | | | |
| | | | | histone H4 [Zea mays] | | | | |
| | | | | &gt:gi\|195658045\|gb\|ACG48490.1\| | | | | |
| | | | | histone H4 [Zea mays] | | | | |
| | | | | &gt:gi\|195658083\|gb\|ACG48509.1\| | | | | |
| | | | | histone H4 [Zea mays] | | | | |
| | | | | &gt:gi\|195658353\|gb\|ACG48644.1\| | | | | |
| | | | | histone H4 [Zea mays] | | | | |
| | | | | &gt:gi\|195658451\|gb\|ACG48693.1\| | | | | |
| | | | | histone H4 [Zea mays] | | | | |
| | | | | &gt:gi\|195659307\|gb\|ACG49121.1\| | | | | |
| | | | | histone H4 [Zea mays] | | | | |
| | | | | &gt:gi\|215740727\|dbj\|BAG97383.1\| | | | | |
| | | | | unnamed protein product | | | | |
| | | | | [Oryza sativa Japonica Group] | | | | |
| | | | | &gt:gi\|215765078\|dbj\|BAG86775.1\| | | | | |
| | | | | unnamed protein product | | | | |
| | | | | [Oryza sativa Japonica Group] | | | | |
| | | | | &gt:gi\|215765094\|dbj\|BAG86791.1\| | | | | |
| | | | | unnamed protein product | | | | |
| | | | | [Oryza sativa Japonica Group] | | | | |
| | | | | &gt:gi\|215765174\|dbj\|BAG86871.1\| | | | | |
| | | | | unnamed protein product | | | | |
| | | | | [Oryza sativa Japonica Group] | | | | |
| | | | | &gt:gi\|215765195\|dbj\|BAG86892.1\| | | | | |
| | | | | unnamed protein product | | | | |
| | | | | [Oryza sativa Japonica Group] | | | | |
| | | | | &gt:gi\|215767370\|dbj\|BAG99598.1\| | | | | |
| | | | | unnamed protein product | | | | |
| | | | | [Oryza sativa Japonica Group] | | | | |
| | | | | &gt:gi\|215767525\|dbj\|BAG99753.1\| | | | | |
| | | | | unnamed protein product | | | | |
| | | | | [Oryza sativa Japonica Group] | | | | |
| | | | | &gt:gi\|222423594\|dbj\|BAH19766.1\| | | | | |
| | | | | AT1G07820 [Arabidopsis thaliana] | | | | |
| | | | | &gt:gi\|222631868\|gb\|EEE64000.1\| | | | | |
| | | | | hypothetical protein OsJ_18829 | | | | |
| | | | | [Oryza sativa Japonica Group] | | | | |
| | | | | &gt:gi\|222641632\|gb\|EEE69764.1\| | | | | |
| | | | | hypothetical protein OsJ_29473 | | | | |
| | | | | [Oryza sativa Japonica Group] | | | | |
| | | | | &gt:gi\|222836752\|gb\|EEE75145.1\| | | | | |
| | | | | histone H4 [Populus trichocarpa] | | | | |
| | | | | &gt:gi\|222836754\|gb\|EEE75147.1\| | | | | |
| | | | | histone H4 [Populus trichocarpa] | | | | |
| | | | | &gt:gi\|222850949\|gb\|EEE88496.1\| | | | | |
| | | | | histone H4 [Populus trichocarpa] | | | | |
| | | | | &gt:gi\|222853756\|gb\|EEE91303.1\| | | | | |
| | | | | histone H4 [Populus trichocarpa] | | | | |
| | | | | &gt:gi\|222853758\|gb\|EEE91305.1\| | | | | |
| | | | | histone H4 [Populus trichocarpa] | | | | |
| | | | | &gt:gi\|222853762\|gb\|EEE91309.1\| | | | | |
| | | | | histone H4 [Populus trichocarpa] | | | | |
| | | | | &gt:gi\|222865366\|gb\|EEF02497.1\| | | | | |
| | | | | histone H4 [Populus trichocarpa] | | | | |
| | | | | &gt:gi\|222865906\|gb\|EEF03037.1\| | | | | |
| | | | | histone H4 [Populus trichocarpa] | | | | |
| | | | | &gt:gi\|222865908\|gb\|EEF03039.1\| | | | | |
| | | | | histone H4 [Populus trichocarpa] | | | | |
| | | | | &gt:gi\|222866490\|gb\|EEF03621.1\| | | | | |
| | | | | histone H4 [Populus trichocarpa] | | | | |
| | | | | &gt:gi\|222870533\|gb\|EEF07664.1\| | | | | |
| | | | | histone H4 [Populus trichocarpa] | | | | |
| | | | | &gt:gi\|222874224\|gb\|EEF11355.1\| | | | | |
| | | | | histone H4 [Populus trichocarpa] | | | | |
| | | | | &gt:gi\|223527428\|gb\|EEF29565.1\| | | | | |
| | | | | histone h4, putative [Ricinus communis] | | | | |
| | | | | &gt:gi\|223528712\|gb\|EEF30724.1\| | | | | |
| | | | | histone h4, putative [Ricinus communis] | | | | |
| | | | | &gt:gi\|223528714\|gb\|EEF30726.1\| | | | | |
| | | | | histone h4, putative [Ricinus communis] | | | | |
| | | | | &gt:gi\|223535654\|gb\|EEF37320.1\| | | | | |
| | | | | histone h4, putative [Ricinus communis] | | | | |
| | | | | &gt:gi\|223541927\|gb\|EEF43473.1\| | | | | |
| | | | | histone h4, putative [Ricinus communis] | | | | |
| | | | | &gt:gi\|223549585\|gb\|EEF51073.1\| | | | | |
| | | | | histone h4, putative [Ricinus communis] | | | | |
| | | | | &gt:gi\|224032847\|gb\|ACN35499.1\| | | | | |
| | | | | unknown [Zea mays] | | | | |
| | | | | &gt:gi\|224285053\|gb\|ACN40254.1\| | | | | |
| | | | | unknown [Picea sitchensis] | | | | |
| | | | | &gt:gi\|238011888\|gb\|ACR36979.1\| | | | | |
| | | | | unknown [Zea mays] | | | | |
| | | | | &gt:gi\|238012310\|gb\|ACR37190.1\| | | | | |
| | | | | unknown [Zea mays] | | | | |
| | | | | &gt:gi\|238014142\|gb\|ACR38 106.1\| | | | | |
| | | | | unknown [Zea mays] | | | | |
| | | | | &gt:gi\|238014264\|gb\|ACR38167.1\| | | | | |
| | | | | unknown [Zea mays] | | | | |
| | | | | &gt:gi\|238014334\|gb\|ACR38202.1\| | | | | |
| | | | | unknown [Zea mays] | | | | |
| | | | | &gt:gi\|238014894\|gb\|ACR38482.1\| | | | | |
| | | | | unknown [Zea mays] | | | | |
| | | | | &gt:gi\|241918866\|gb\|EER92010.1\| | | | | |
| | | | | hypothetical protein SORBIDRAFT_01g030460 | | | | |
| | | | | [Sorghum bicolor] | | | | |
| | | | | &gt:gi\|241922478\|gb\|EER95622.1\| | | | | |
| | | | | hypothetical protein SORBIDRAFT_01g049250 | | | | |
| | | | | [Sorghum bicolor] | | | | |
| | | | | &gt:gi\|241923627\|gb\|EER96771.1\| | | | | |
| | | | | hypothetical protein SORBIDRAFT_02g025440 | | | | |
| | | | | [Sorghum bicolor] | | | | |
| | | | | &gt:gi\|241926180\|gb\|EER99324.1\| | | | | |
| | | | | hypothetical protein SORBIDRAFT_02g032240 | | | | |
| | | | | [Sorghum bicolor] | | | | |
| | | | | &gt:gi\|241927105\|gb\|EES00250.1\| | | | | |
| | | | | hypothetical protein SORBIDRAFT_03g004840 | | | | |
| | | | | [Sorghum bicolor] | | | | |
| | | | | &gt:gi\|241927106\|gb\|EES00251.1\| | | | | |
| | | | | hypothetical protein SORBIDRAFT_03g004870 | | | | |
| | | | | [Sorghum bicolor] | | | | |
| | | | | &gt:gi\|241928571\|gb\|EES01716.1\| | | | | |
| | | | | hypothetical protein SORBIDRAFT_03g039090 | | | | |
| | | | | [Sorghum bicolor] | | | | |
| | | | | &gt:gi\|241929261\|gb\|EES02406.1\| | | | | |
| | | | | hypothetical protein SORBIDRAFT_03g004890 | | | | |
| | | | | [Sorghum bicolor] | | | | |
| | | | | &gt:gi\|241932574\|gb\|EES05719.1\| | | | | |
| | | | | hypothetical protein SORBIDRAFT_04g031620 | | | | |
| | | | | [Sorghum bicolor] | | | | |
| | | | | &gt:gi\|241939578\|gb\|EES12723.1\| | | | | |
| | | | | hypothetical protein SORBIDRAFT_06g026490 | | | | |
| | | | | [Sorghum bicolor] | | | | |
| | | | | &gt:gi\|241945217\|gb\|EES18362.1\| | | | | |
| | | | | hypothetical protein SORBIDRAFT_09g022920 | | | | |
| | | | | [Sorghum bicolor] | | | | |
| | | | | &gt:gi\|255625991\|gb\|ACU13340.1\| | | | | |
| | | | | unknown [Glycine max] | | | | |
| | | | | &gt:gi\|255673853\|dbj\|BAF06643.2\| | | | | |
| | | | | Os01g0835900 [Oryza sativa Japonica Group] | | | | |
| | | | | &gt:gi\|255677871\|dbj\|BAH93978.1\| | | | | |
| | | | | Os07g0549900 [Oryza sativa Japonica Group] | | | | |
| | | | | &gt:gi\|297310482\|gb\|EFH40906.1\| | | | | |
| | | | | hypothetical protein ARALYDRAFT_496101 | | | | |
| | | | | [Arabidopsis lyrata subsp. lyrata] | | | | |
| | | | | &gt:gi\|297321595\|gb\|EFH52016.1\| | | | | |
| | | | | hypothetical protein ARALYDRAFT_484971 | | | | |
| | | | | [Arabidopsis lyrata subsp. lyrata] | | | | |
| | | | | &gt:gi\|297323319\|gb\|EFH53740.1\| | | | | |
| | | | | hypothetical protein ARALYDRAFT_485010 | | | | |
| | | | | [Arabidopsis lyrata subsp. lyrata] | | | | |
| | | | | &gt:gi\|297323776\|gb\|EFH54197.1\| | | | | |
| | | | | hypothetical protein ARALYDRAFT_485763 | | | | |
| | | | | [Arabidopsis lyrata subsp. lyrata] | | | | |
| | | | | &gt:gi\|297326845\|gb\|EFH57265.1\| | | | | |
| | | | | hypothetical protein ARALYDRAFT_481787 | | | | |
| | | | | [Arabidopsis lyrata subsp. lyrata] | | | | |
| | | | | &gt:gi\|326488189\|dbj\|BAJ89933.1\| | | | | |
| | | | | predicted protein | | | | |
| | | | | [Hordeum vulgare subsp. vulgare] | | | | |
| | | | | &gt:gi\|326489645\|dbj\|BAK01803.1\| | | | | |
| | | | | predicted protein | | | | |
| | | | | [Hordeum vulgare subsp. vulgare] | | | | |
| | | | | &gt:gi\|326490127\|dbj\|BAJ94137.1\| | | | | |
| | | | | predicted protein | | | | |
| | | | | [Hordeum vulgare subsp. vulgare] | | | | |
| | | | | &gt:gi\|326497283\|dbj\|BAK02226.1\| | | | | |
| | | | | predicted protein | | | | |
| | | | | [Hordeum vulgare subsp. vulgare] | | | | |
| | | | | &gt:gi\|326498581\|dbj\|BAJ98718.1\| | | | | |
| | | | | predicted protein | | | | |
| | | | | [Hordeum vulgare subsp. vulgare] | | | | |
| | | | | &gt:gi\|326500352\|dbj\|BAK06265.1\| | | | | |
| | | | | predicted protein | | | | |
| | | | | [Hordeum vulgare subsp. vulgare] | | | | |
| | | | | &gt:gi\|326503956\|dbj\|BAK02764.1\| | | | | |
| | | | | predicted protein | | | | |
| | | | | [Hordeum vulgare subsp. vulgare] | | | | |
| | | | | &gt:gi\|326505870\|dbj\|BAJ91174.1\| | | | | |
| | | | | predicted protein | | | | |
| | | | | [Hordeum vulgare subsp. vulgare] | | | | |
| | | | | &gt:gi\|326505922\|dbj\|BAJ91200.1\| | | | | |
| | | | | predicted protein | | | | |
| | | | | [Hordeum vulgare subsp. vulgare] | | | | |
| | | | | &gt:gi\|326506492\|dbj\|BAJ86564.1\| | | | | |
| | | | | predicted protein | | | | |
| | | | | [Hordeum vulgare subsp. vulgare] | | | | |
| | | | | &gt:gi\|326506520\|dbj\|BAJ86578.1\| | | | | |
| | | | | predicted protein | | | | |
| | | | | [Hordeum vulgare subsp. vulgare] | | | | |
| | | | | &gt:gi\|326522106\|dbj\|BAK04181.1\| | | | | |
| | | | | predicted protein | | | | |
| | | | | [Hordeum vulgare subsp. vulgare] | | | | |
| | | | | &gt:gi\|326523419\|dbj\|BAJ88750.1\| | | | | |
| | | | | predicted protein | | | | |
| | | | | [Hordeum vulgare subsp. vulgare] | | | | |
| | | | | &gt:gi\|326524814\|dbj\|BAK04343.1\| | | | | |
| | | | | predicted protein | | | | |
| | | | | [Hordeum vulgare subsp. vulgare] | | | | |
| | | | | &gt:gi\|326529405\|dbj\|BAK04649.1\| | | | | |
| | | | | predicted protein | | | | |
| | | | | [Hordeum vulgare subsp. vulgare] | | | | |
| | | | | &gt:gi\|326530001\|dbj\|BAK08280.1\| | | | | |
| | | | | predicted protein | | | | |
| | | | | [Hordeum vulgare subsp. vulgare] | | | | |
| | | | | &gt:gi\|326532258\|dbj\|BAK05058.1\| | | | | |
| | | | | predicted protein | | | | |
| | | | | [Hordeum vulgare subsp. vulgare] | | | | |
| | | | | &gt:gi\|326532458\|dbj\|BAK05158.1\| | | | | |
| | | | | predicted protein | | | | |
| | | | | [Hordeum vulgare subsp. vulgare] | | | | |
| | | | | &gt:gi\|326533346\|dbj\|BAJ93645.1\| | | | | |
| | | | | predicted protein | | | | |
| | | | | [Hordeum vulgare subsp. vulgare] | | | | |
| | | | | &gt:gi\|330253071\|gb\|AEC08165.1\| | | | | |
| | | | | histone H4 [Arabidopsis thaliana] | | | | |
| | | | | &gt:gi\|330318553\|gb\|AEC10949.1\| | | | | |
| | | | | histone H4 [Camellia sinensis] | | | | |
| | | | | &gt:gi\|332009837\|gb\|AED97220.1\| | | | | |
| | | | | histone H4 [Arabidopsis thaliana] | | | | |
| | | | | &gt:gi\|332009877\|gb\|AED97260.1\| | | | | |
| | | | | histone H4 [Arabidopsis thaliana] | | | | |
| | | | | &gt:gi\|332190037\|gb\|AEE28158.1\| | | | | |
| | | | | histone H4 [Arabidopsis thaliana] | | | | |
| | | | | &gt:gi\|332190066\|gb\|AEE28187.1\| | | | | |
| | | | | histone H4 [Arabidopsis thaliana] | | | | |
| | | | | &gt:gi\|332190067\|gb\|AEE28188.1\| | | | | |
| | | | | histone H4 [Arabidopsis thaliana] | | | | |
| | | | | &gt:gi\|332644570\|gb\|AEE78091.1\| | | | | |
| | | | | histone H4 [Arabidopsis thaliana] | | | | |
| | | | | &gt:gi\|332644625\|gb\|AEE78 146.1\| | | | | |
| | | | | histone H4 [Arabidopsis thaliana] | | | | |
| | | | | &gt:gi\|332645612\|gb\|AEE79133.1\| | | | | |
| | | | | histone H4 [Arabidopsis thaliana] | | | | |
| | | | | &gt:gi\|225838\|prf\|\|1314298A | | | | |
| | | | | histone H4 | | | | |
| | | AAT08725 | 47027019 | histone H4 [Hyacinthus orientalis] | 0.96875 | Hyacinthus orientalis | 3076 | 4452 |
| | | ADL36649 | 302398708 | C3HL domain class transcription factor | 0.96875 | Malus × domestica | 3077 | 4453 |
| | | | | [Malus × domestica] | | | | |
| | | | | &gt:gi\|302398719\|gb\|ADL36654.1\| | | | | |
| | | | | C3HL domain class transcription factor | | | | |
| | | | | [Malus × domestica] | | | | |
| | 15-33 | NP_001044757 | 297597935 | Os01g0840100 [Oryza sativa Japonica Group] | 1 | Oryza sativa Japonica Group | 3078 | 4454 |
| | | | | &gt:gi\|15623835\|dbj\|BAB67894.1\| putative | | | | |
| | | | | HSP70 [Oryza sativa Japonica Group] | | | | |
| | | | | &gt:gi\|21104622\|dbj\|BAB93214.1\| putative | | | | |
| | | | | HSP70 [Oryza sativa Japonica Group] | | | | |
| | | | | &gt:gi\|113534288\|dbj\|BAF06671.1\| Os01g0840100 | | | | |
| | | | | [Oryza sativa Japonica Group] | | | | |
| | | | | &gt:gi\|125572585\|gb\|EAZ14100.1\| hypothetical | | | | |
| | | | | protein OsJ_04024 [Oryza sativa Japonica Group] | | | | |
| | | | | &gt:gi\|215769289\|dbj\|BAH01518.1\| unnamed | | | | |
| | | | | protein product [Oryza sativa Japonica Group] | | | | |
| | | | | &gt:gi\|306416013\|gb\|ADM86881.1\| 70 kDa heat | | | | |
| | | | | shock protein [Oryza sativa Japonica Group] | | | | |
| | | | | &gt:gi\|313575779\|gb\|ADR66969.1\| 70 kDa heat | | | | |
| | | | | shock protein [Oryza sativa Japonica Group] | | | | |
| | | XP_002884912 | 297834059 | hypothetical protein ARALYDRAFT_897465 | 0.94444 | Arabidopsis lyrata subsp. lyrata | 3079 | 4455 |
| | | | | [Arabidopsis lyrata subsp. lyrata] | | | | |
| | | | | &gt:gi\|297330752\|gb\|EFH61171.1\| | | | | |
| | | | | hypothetical protein ARALYDRAFT_897465 | | | | |
| | | | | [Arabidopsis lyrata subsp. lyrata] | | | | |
| | 703-721 | NP_001144808 | 226501057 | hypothetical protein LOC100277884 [Zea mays] | 1 | Zea mays | 3080 | 4456 |
| | | | | &gt:gi\|195647306\|gb\|ACG43121.1\| | | | | |
| | | | | hypothetical protein [Zea mays] | | | | |
| | 152-170 | XP_002466218 | 242037646 | hypothetical protein SORBIDRAFT_01g003720 | 1 | Sorghum bicolor | 3081 | 4457 |
| | | | | [Sorghum bicolor] | | | | |
| | | | | &gt:gi\|241920072\|gb\|EER93216.1\| | | | | |
| | | | | hypothetical protein SORBIDRAFT_01g003720 | | | | |
| | | | | [Sorghum bicolor] | | | | |
| | | NP_001144602 | 226501987 | hypothetical protein LOC100277618 [Zea mays] | 0.952489 | Zea mays | 3082 | 4458 |
| | | | | &gt:gi\|195644458\|gb\|ACG41697.1\| | | | | |
| | | | | hypothetical protein [Zea mays] | | | | |
| | | CAE05414 | 61656646 | OSJNBa0035I04.2 [Oryza sativa Japonica Group] | 0.834842 | Oryza sativa Japonica Group | 3083 | 4459 |
| | | | | &gt:gi\|38605918\|emb\|CAE05953.3\| | | | | |
| | | | | OSJNBb0088C09.12 [Oryza sativa Japonica Group] | | | | |
| | | | | &gt:gi\|116309409\|emb\|CAH66485.1\| | | | | |
| | | | | OSIGBa0076I14.6 [Oryza sativa Indica Group] | | | | |
| | | EEC77275 | 54362548 | hypothetical protein OsI_15905 | 0.837104 | Oryza sativa Indica Group | 3084 | |
| | | | | [Oryza sativa Indica Group] | | | | |
| | | NP_001052798 | 297602722 | Os04g0423700 [Oryza sativa Japonica Group] | 0.834842 | Oryza sativa Japonica Group | 3085 | 4460 |
| | | | | &gt:gi\|255675459\|dbj\|BAF14712.2\| | | | | |
| | | | | Os04g0423700 [Oryza sativa Janonica Group] | | | | |
| | | ACN34295 | 224030438 | unknown [Zea mays] | 0.769231 | Zea mays | 3086 | 4461 |
| | | EEC73359 | 54362548 | hypothetical protein OsI_07583 | 0.794118 | Oryza sativa Indica Group | 3087 | |
| | | | | [Oryza sativa Indica Group] | | | | |
| | | XP_002453960 | 242065341 | hypothetical protein SORBIDRAFT_04g022230 | 0.721719 | Sorghum bicolor | 3088 | 4462 |
| | | | | [Sorghum bicolor] | | | | |
| | | | | &gt:gi\|241933791\|gb\|EES06936.1\| | | | | |
| | | | | hypothetical protein SORBIDRAFT_04g022230 | | | | |
| | | | | [Sorghum bicolor] | | | | |
| Predicted | 167-186 | ACR34837 | 238007603 | unknown [Zea mays] | 1 | Zea mays | 3089 | 4463 |
| siRNA | | | | | | | | |
| 59799 | | | | | | | | |
| | | ACF84683 | 194701197 | unknown [Zea mays] | 1 | Zea mays | 3090 | 4464 |
| | | | | &gt:gi\|195638498\|gb\|ACG38717.1\| | | | | |
| | | | | monoglyceride lipase [Zea mays] | | | | |
| | | NP_001136909 | 219363598 | hypothetical protein LOC100217066 [Zea mays] | 0.838235 | Zea mays | 3091 | 4465 |
| | | | | &gt:gi\|194697568\|gb\|ACF82868.1\| | | | | |
| | | | | unknown [Zea mays] | | | | |
| | | EEC70511 | 54362548 | hypothetical protein OsI_01608 | 0.814706 | Oryza sativa Indica Group | 3092 | |
| | | | | [Oryza sativa Indica Group] | | | | |
| | | XP_002439655 | 242087644 | hypothetical protein SORBIDRAFT_09g018060 | 0.752941 | Sorghum bicolor | 3093 | 4466 |
| | | | | [Sorghum bicolor] | | | | |
| | | | | &gt:gi\|241944940\|gb\|EES18085.1\| | | | | |
| | | | | hypothetical protein SORBIDRAFT_09g018060 | | | | |
| | | | | [Sorghum bicolor] | | | | |
| | | BAK01593 | 326489218 | predicted protein | 0.738235 | Hordeum vulgare subsp. vulgare | 3094 | 4467 |
| | | | | [Hordeum vulgare subsp. vulgare] | | | | |
| | | BAJ99202 | 326503153 | predicted protein | 0.738235 | Hordeum vulgare subsp. vulgare | 3095 | 4468 |
| | | | | [Hordeum vulgare subsp. vulgare] | | | | |
| | | NP_001055317 | 115463434 | Os05g0363100 [Oryza sativa Japonica Group] | 0.720588 | Oryza sativa Japonica Group | 3096 | 4469 |
| | | | | &gt:gi\|54287660\|gb\|AAV31404.1\| | | | | |
| | | | | putative phospholipase | | | | |
| | | | | [Oryza sativa Japonica Group] | | | | |
| | | | | &gt:gi\|113578868\|dbj\|BAF17231.1\| | | | | |
| | | | | Os05g0363100 [Oryza sativa Japonica Group] | | | | |
| | | EEE63431 | 54398660 | hypothetical protein OsJ_18244 | 0.720588 | Oryza sativa Japonica Group | 3097 | |
| | | | | [Oryza sativa Japonica Group] | | | | |
| | 255-274 | XP_002455548 | 242052804 | hypothetical protein SORBIDRAFT_03g012970 | 1 | Sorghum bicolor | 3098 | 4470 |
| | | | | [Sorghum bicolor] | | | | |
| | | | | &gt:gi\|241927523\|gb\|EES00668.1\| | | | | |
| | | | | hypothetical protein SORBIDRAFT_03g012970 | | | | |
| | | | | [Sorghum bicolor] | | | | |
| | 660-679 | XP_002444285 | 242079032 | hypothetical protein SORBIDRAFT_07g019500 | 1 | Sorghum bicolor | 3099 | 4471 |
| | | | | [Sorghum bicolor] | | | | |
| | | | | &gt:gi\|241940635\|gb\|EES13780.1\| | | | | |
| | | | | hypothetical protein SORBIDRAFT_07g019500 | | | | |
| | | | | [Sorghum bicolor] | | | | |
| | | NP_001149164 | 226533573 | F-box domain containing protein [Zea mays] | 0.795154 | Zea mays | 3100 | 4472 |
| | | | | &gt:gi\|195625194\|gb\|ACG34427.1\| F-box domain | | | | |
| | | | | containing protein [Zea mays] | | | | |
| Predicted | 344-363 | NP_001132616 | 239047681 | hypothetical protein LOC100194090 [Zea mays] | 1 | Zea mays | 3101 | 4473 |
| siRNA | | | | &gt:gi\|194703996\|gb\|ACF86082.1\| | | | | |
| 59800 | | | | unknown [Zea mays] | | | | |
| | | | | &gt:gi\|238908725\|gb\|ACF81540.21 | | | | |
| | | | | unknown [Zea mays] | | | | |
| | | NP_001147445 | 226501241 | myb-related protein Hv33 [Zea mays] | 0.960725 | Zea mays | 3102 | 4474 |
| | | | | &gt:gi\|195611444\|gb\|ACG27552.1\| | | | | |
| | | | | myb-related protein Hv33 [Zea mays] | | | | |
| | | XP_002465218 | 242035646 | hypothetical protein SORBIDRAFT_01g034270 | 0.864048 | Sorghum bicolor | 3103 | 4475 |
| | | | | [Sorghum bicolor] | | | | |
| | | | | &gt:gi\|241919072\|gb\|EER92216.1\| | | | | |
| | | | | hypothetical protein SORBIDRAFT_01g034270 | | | | |
| | | | | [Sorghum bicolor] | | | | |
| Predicted | 1526-1544 | NP_001143089 | 226498753 | hypothetical protein LOC100275564 [Zea mays] | 1 | Zea mays | 3104 | 4476 |
| siRNA | | | | &gt:gi\|195614134\|gb\|ACG28897.1\| | | | | |
| 59820 | | | | hypothetical protein [Zea mays] | | | | |
| Predicted | 871-891 | XP_002458387 | 242058482 | hypothetical protein SORBIDRAFT_03g032650 | 1 | Sorghum bicolor | 3105 | 4477 |
| siRNA | | | | [Sorghum bicolor] | | | | |
| 59918 | | | | &gt:gi\|241930362\|gb\|EES03507.1\| | | | | |
| | | | | hypothetical protein SORBIDRAFT_03g032650 | | | | |
| | | | | [Sorghum bicolor] | | | | |
| | | NP_001169333 | 293332220 | hypothetical protein LOC100383200 [Zea mays] | 0.862136 | Zea mays | 3106 | 4478 |
| | | | | &gt:gi\|224028761\|gb\|ACN33456.1\| | | | | |
| | | | | unknown [Zea mays] | | | | |
| | | EAY75564 | 54362548 | hypothetical protein OsI_03468 | 0.780583 | Oryza sativa Indica Group | 3107 | |
| | | | | [Oryza sativa Indica Group] | | | | |
| | | NP_001044038 | 115439516 | Os01g0710200 [Oryza sativa Japonica Group] | 0.778641 | Oryza sativa Japonica Group | 3108 | 4479 |
| | | | | &gt:gi\|56784137\|dbj\|BAD81522.1\| polyamine | | | | |
| | | | | oxidase-like [Oryza sativa Japonica Group] | | | | |
| | | | | &gt:gi\|113533569\|dbj\|BAF05952.1\| Os01g0710200 | | | | |
| | | | | [Oryza sativa Japonica Group] | | | | |
| | | | | &gt:gi\|125571772\|gb\|EAZ13287.1\| | | | | |
| | | | | hypothetical protein OsJ_03212 | | | | |
| | | | | [Oryza sativa Japonica Group] | | | | |
| | | BAK01686 | 326489405 | predicted protein | 0.765049 | Hordeum vulgare subsp. vulgare | 3109 | 4480 |
| | | | | [Hordeum vulgare subsp. vulgare] | | | | |
| Predicted | 20-37 | AAY42386 | 65335862 | inorganic phosphate transporter 2 | 1 | Zea mays | 3110 | 4481 |
| siRNA | | | | [Zea mays] | | | | |
| 60421 | | ACF85228 | 194702287 | unknown [Zea mays] | 0.987085 | Zea mays | 3111 | 4482 |
| | | | | &gt:gi\|238007276\|gb\|ACR34673.1\| | | | | |
| | | | | unknown [Zea mays] | | | | |
| | | NP_001105269 | 162458547 | phosphate transporter protein 1 [Zea mays] | 0.987085 | Zea mays | 3112 | 4483 |
| | | | | &gt:gi\|49066604\|gb\|AAT51690.1\| | | | | |
| | | | | phosphate transport protein [Zea mays] | | | | |
| | | ACF84347 | 194700525 | unknown [Zea mays] | 0.98524 | Zea mays | 3113 | 4484 |
| | | AAY42388 | 65335893 | inorganic phosphate transporter 4 | 0.98524 | Zea mays | 3114 | 4485 |
| | | | | [Zea mays] | | | | |
| | | XP_002464558 | 242034326 | hypothetical protein SORBIDRAFT_01g020570 | 0.948339 | Sorghum bicolor | 3115 | 4486 |
| | | | | [Sorghum bicolor] | | | | |
| | | | | &gt:gi\|241918412\|gb\|EER91556.1\| | | | | |
| | | | | hypothetical protein SORBIDRAFT_01g020570 | | | | |
| | | | | [Sorghum bicolor] | | | | |
| | | NP_001105816 | 162461218 | phosphate transporter protein2 [Zea mays] | 0.944649 | Zea mays | 3116 | 4487 |
| | | | | &gt:gi\|65335848\|gb\|AAY42385.1\| | | | | |
| | | | | inorganic phosphate transporter 1 | | | | |
| | | | | [Zea mays] | | | | |
| | | | | &gt:gi\|223944389\|gb\|ACN26278.1\| | | | | |
| | | | | unknown [Zea mays] | | | | |
| | | ACG38622 | 195638307 | inorganic phosphate transporter | 0.931734 | Zea mays | 3117 | 4488 |
| | | | | 1-4 [Zea mays] | | | | |
| | | AAO72437 | 29367130 | phosphate transporter HvPT4 | 0.841328 | Hordeum vulgare subsp. vulgare | 3118 | 4489 |
| | | | | [Hordeum vulgare subsp. vulgare] | | | | |
| | | | | &gt:gi\|29367133\|gb\|AAO72438.1\| | | | | |
| | | | | phosphate transporter HvPT4 | | | | |
| | | | | [Hordeum vulgare subsp. vulgare] | | | | |
| | | NP_001064708 | 115482229 | Os10g0444700 [Oryza sativa Japonica Group] | 0.845018 | Oryza sativa Japonica Group | 3119 | 4490 |
| | | | | &gt:gi\|75299854\|sp\|Q8H6G8.1\|PHT18_ORYSJ | | | | |
| | | | | RecName: Full = Probable inorganic phosphate | | | | |
| | | | | transporter 1-8; Short = OsPT8; Short = OsPht1; 8; | | | | |
| | | | | AltName: Full = H(+)/Pi cotransporter | | | | |
| | | | | &gt:gi\|23600468\|gb\|AAN39049.1\| | | | | |
| | | | | putative phosphate transporter OsPT8 | | | | |
| | | | | [Oryza sativa Japonica Group] | | | | |
| | | | | &gt:gi\|31432353\|gb\|AAP53996.1\| | | | | |
| | | | | phosphate: H+ symporter family protein, expressed | | | | |
| | | | | [Oryza sativa Japonica Group] | | | | |
| | | | | &gt:gi\|78708737\|gb\|ABB47712.1\| | | | | |
| | | | | phosphate: H+ symporter family protein, expressed | | | | |
| | | | | [Oryza sativa Japonica Group] | | | | |
| | | | | &gt:gi\|113639317\|dbj\|BAF26622.1\| | | | | |
| | | | | Os10g0444700 [Oryza sativa Japonica Group] | | | | |
| | | | | &gt:gi\|125532137\|gb\|EAY78702.1\| | | | | |
| | | | | hypothetical protein OsI_33803 | | | | |
| | | | | [Oryza sativa Indica Group] | | | | |
| | | | | &gt:gi\|125574950\|gb\|EAZ16234.1\| | | | | |
| | | | | hypothetical protein OsJ_31686 | | | | |
| | | | | [Oryza sativa Japonica Group] | | | | |
| | 531-548 | NP_001151553 | 226497123 | nucleolar protein, Nop52 containing | 1 | Zea mays | 3120 | 4491 |
| | | | | protein [Zea mays] | | | | |
| | | | | &gt:gi\|195647654\|gb\|ACG43295.1\| | | | | |
| | | | | nucleolar protein, Nop52 containing | | | | |
| | | | | protein [Zea mays] | | | | |
| | | NP_001168446 | 293336366 | hypothetical protein LOC100382218 [Zea mays] | 0.815742 | Zea mays | 3121 | 4492 |
| | | | | &gt:gi\|223948351\|gb\|ACN28259.1\| | | | | |
| | | | | unknown [Zea mays] | | | | |
| | | AAL87179 | 19387261 | unknown [Oryza sativa Japonica Group] | 0.711986 | Oryza sativa Japonica Group | 3122 | 4493 |
| | | | | &gt:gi\|38346404\|emb\|CAE04237.2\| | | | | |
| | | | | OSJNBa0011F23.10 [Oryza sativa Japonica Group] | | | | |
| | | | | &gt:gi\|222629700\|gb\|EEE61832.1\| | | | | |
| | | | | hypothetical protein OsJ_16477 | | | | |
| | | | | [Oryza sativa Japonica Group] | | | | |
| | | NP_001054129 | 115461057 | Os04g0658600 [Oryza sativa Japonica Group] | 0.703041 | Oryza sativa Japonica Group | 3123 | 4494 |
| | | | | &gt:gi\|113565700\|dbj\|BAF16043.1\| | | | | |
| | | | | Os04g0658600 [Oryza sativa Japonica Group] | | | | |
| | | CAH67793 | 116310844 | OSIGBa0132E09-OSIGBa0108L24.7 | 0.70483 | Oryza sativa Indica Group | 3124 | 4495 |
| | | | | [Oryza sativa Indica Group] | | | | |
| | | EAY95889 | 54362548 | hypothetical protein OsI_17752 | 0.703041 | Oryza sativa Indica Group | 3125 | |
| | | | | [Oryza sativa Indica Group] | | | | |
| | 117-134 | NP_001148391 | 226531629 | nitrate-induced NOI protein [Zea mays] | 1 | Zea mays | 3126 | 4496 |
| | | | | &gt:gi\|195618920\|gb\|ACG31290.1\| | | | | |
| | | | | nitrate-induced NOI protein [Zea mays] | | | | |
| | | XP_002448552 | 242077231 | hypothetical protein SORBIDRAFT_06g028920 | 0.858696 | Sorghum bicolor | 3127 | 4497 |
| | | | | [Sorghum bicolor] | | | | |
| | | | | &gt:gi\|241939735\|gb\|EES12880.1\| | | | | |
| | | | | hypothetical protein SORBIDRAFT_06g028920 | | | | |
| | | | | [Sorghum bicolor] | | | | |
| | | NP_001053908 | 115460615 | Os04g0620600 [Oryza sativa Japonica Group] | 0.717391 | Oryza sativa Japonica Group | 3128 | 4498 |
| | | | | &gt:gi\|38344338\|emb\|CAE02154.2\| | | | | |
| | | | | OSJNBa0058K23.20 [Oryza sativa Japonica Group] | | | | |
| | | | | &gt:gi\|113565479\|dbj\|BAF15822.1\| Os04g0620600 | | | | |
| | | | | [Oryza sativa Japonica Group] | | | | |
| | | | | &gt:gi\|116309950\|emb\|CAH66981.1\| | | | | |
| | | | | H0714H04.8 [Oryza sativa Indica Group] | | | | |
| | | | | &gt:gi\|215768265\|dbj\|BAH00494.1\| | | | | |
| | | | | unnamed protein product | | | | |
| | | | | [Oryza sativa Japonica Group] | | | | |
| | 986-1003 | ACF84537 | 194700905 | unknown [Zea mays] | 1 | Zea mays | 3129 | 4499 |
| | | AAM13441 | 20152972 | similar to H. sapiens NNP-1/Nop52 AP001752 | 0.710909 | Hordeum vulgare subsp. vulgare | 3130 | 4500 |
| | | | | (Score = 92; E = 9e−18) | | | | |
| | | | | [Hordeum vulgare subsp. vulgare] | | | | |
| | 3196-3213 | XP_002443497 | 242086143 | hypothetical protein SORBIDRAFT_08g020610 | 1 | Sorghum bicolor | 3131 | 4501 |
| | | | | [Sorghum bicolor] | | | | |
| | | | | &gt:gi\|241944190\|gb\|EES17335.1\| | | | | |
| | | | | hypothetical protein SORBIDRAFT_08g020610 | | | | |
| | | | | [Sorghum bicolor] | | | | |
| | | NP_001106067 | 239985666 | H+-translocating pyrophosphatase | 0.981227 | Zea mays | 3132 | 4502 |
| | | | | [Zea mays] | | | | |
| | | | | &gt:gi\|117622272\|gb\|ABK51382.1\| | | | | |
| | | | | H+-translocating pyrophosphatase | | | | |
| | | | | [Zea mays] | | | | |
| | | EEC73348 | 54362548 | hypothetical protein OsI_07556 | 0.946183 | Oryza sativa Indica Group | 3133 | |
| | | | | [Oryza sativa Indica Group] | | | | |
| | | NP_001047051 | 115446542 | Os02g0537900 [Oryza sativa Japonica Group] | 0.94368 | Oryza sativa Japonica Group | 3134 | 4503 |
| | | | | &gt:gi\|50251984\|dbj\|BAD27918.1\| | | | | |
| | | | | putative vacuolar-type H+-translocating | | | | |
| | | | | inorganic pyrophosphatase | | | | |
| | | | | [Oryza sativa Japonica Group] | | | | |
| | | | | &gt:gi\|50252660\|dbj\|BAD28829.1\| | | | | |
| | | | | putative vacuolar-type H+-translocating | | | | |
| | | | | inorganic pyrophosphatase | | | | |
| | | | | [Oryza sativa Japonica Group] | | | | |
| | | | | &gt:gi\|113536582\|dbj\|BAF08965.1\| | | | | |
| | | | | Os02g0537900 [Oryza sativa Japonica Group] | | | | |
| | | | | &gt:gi\|222623005\|gb\|EEE57137.1\| | | | | |
| | | | | hypothetical protein OsJ_07039 | | | | |
| | | | | [Oryza sativa Japonica Group] | | | | |
| | | BAJ95133 | 326500933 | predicted protein | 0.918648 | Hordeum vulgare subsp. vulgare | 3135 | 4504 |
| | | | | [Hordeum vulgare subsp. vulgare] | | | | |
| | | Q9FWR2 | | RecName: Full = Pyrophosphate-energized | 0.862328 | Arabidopsis thaliana | 3136 | |
| | | | | membrane proton pump 3; | | | | |
| | | | | AltName: Full = AVP1-like protein 2; | | | | |
| | | | | AltName: Full = Pyrophosphate-energized inorganic | | | | |
| | | | | pyrophosphatase 3; Short = H(+)-PPase 3 | | | | |
| | | | | &gt:gi\|9954727\|gb\|AAG09080.1\|AC026237_1 | | | | |
| | | | | Putative vacuolar-type H+-translocating | | | | |
| | | | | inorganic pyrophosphatase | | | | |
| | | | | [Arabidopsis thaliana] | | | | |
| | | NP_173122 | 334182630 | Pyrophosphate-energized membrane proton | 0.862328 | Arabidopsis thaliana | 3137 | 4505 |
| | | | | pump 3 [Arabidopsis thaliana] | | | | |
| | | | | &gt:gi\|332191375\|gb\|AEE29496.1\| | | | | |
| | | | | Pyrophosphate-energized membrane proton | | | | |
| | | | | pump 3 [Arabidopsis thaliana] | | | | |
| | | XP_002265811 | 225443360 | PREDICTED: hypothetical protein [Vitis vinifera] | 0.87234 | Vitis vinifera | 3138 | 4506 |
| | | | | &gt:gi\|297735766\|emb\|CBI18453.3\| | | | | |
| | | | | unnamed protein product [Vitis vinifera] | | | | |
| | | XP_002887762 | 297839760 | vacuolar H+-pyrophosphatase 2 | 0.862328 | Arabidopsis lyrata subsp. lyrata | 3139 | 4507 |
| | | | | [Arabidopsis lyrata subsp. lyrata] | | | | |
| | | | | &gt:gi\|297333603\|gb\|EFH64021.1\| | | | | |
| | | | | vacuolar H+-pyrophosphatase 2 | | | | |
| | | | | [Arabidopsis lyrata subsp. lyrata] | | | | |
| | | NP_565195 | 145337727 | pyrophosphate-energized membrane proton | 0.85607 | Arabidopsis thaliana | 3140 | 4508 |
| | | | | pump 2 [Arabidopsis thaliana] | | | | |
| | | | | &gt:gi\|186496309lref\|NP_001117619.1\| | | | | |
| | | | | pyrophosphate-energized membrane proton | | | | |
| | | | | pump 2 [Arabidopsis thaliana] | | | | |
| | | | | &gt:gi\|83287950\|sp\|Q56ZN6.2\|AVP2_ARATH | | | | |
| | | | | RecName: Full = Pyrophosphate-energized | | | | |
| | | | | membrane proton pump 2; | | | | |
| | | | | AltName: Full = AVP1-like protein 1; | | | | |
| | | | | AltName: Full = Pyrophosphate-energized inorganic | | | | |
| | | | | pyrophosphatase 2; Short = H(+)-PPase 2; | | | | |
| | | | | AltName: Full = Vacuolar proton pyrophosphatase 2 | | | | |
| | | | | &gt:gi\|7024455\|dbj\|BAA92151.1\| | | | | |
| | | | | vacuolar-pyrophosphatase like protein | | | | |
| | | | | [Arabidopsis thaliana] | | | | |
| | | | | &gt:gi\|15450810\|gb\|AAK96676.1\| | | | | |
| | | | | Similar to vacuolar H+-pyrophosphatase | | | | |
| | | | | [Arabidopsis thaliana] | | | | |
| | | | | &gt:gi\|34098827\|gb\|AAQ56796.1\| | | | | |
| | | | | At1g78920 [Arabidopsis thaliana] | | | | |
| | | | | &gt:gi\|332198056\|gb\|AEE36177.1\| | | | | |
| | | | | pyrophosphate-energized membrane proton | | | | |
| | | | | pump 2 [Arabidopsis thaliana] | | | | |
| | | | | &gt:gi\|332198057\|gb\|AEE36178.1\| | | | | |
| | | | | pyrophosphate-energized membrane proton | | | | |
| | | | | pump 2 [Arabidopsis thaliana] | | | | |
| Predicted | 35-54 | NP_001167652 | 293331418 | hypothetical protein LOC100381282 | 1 | Zea mays | 3141 | 4509 |
| siRNA | | | | [Zea mays] | | | | |
| 60533 | | | | &gt:gi\|194708280\|gb\|ACF88224.1\| | | | | |
| | | | | unknown [Zea mays] | | | | |
| | | NP_001060508 | 115473818 | Os07g0656900 [Oryza sativa Japonica Group] | 0.793451 | Oryza sativa Japonica Group | 3142 | 4510 |
| | | | | &gt:gi\|113612044\|dbj\|BAF22422.1\| | | | | |
| | | | | Os07g0656900 [Oryza sativa Japonica Group] | | | | |
| | | EEE67734 | 54398660 | hypothetical protein OsJ_25423 | 0.793451 | Oryza sativa Japonica Group | 3143 | |
| | | | | [Oryza sativa Japonica Group] | | | | |
| | | EEC82601 | 54362548 | hypothetical protein OsI_27169 | 0.7733 | Oryza sativa Indica Group | 3144 | |
| | | | | [Oryza sativa Indica Group] | | | | |
| | 220-239 | XP_002463867 | 242032944 | hypothetical protein SORBIDRAFT_01g007880 | 1 | Sorghum bicolor | 3145 | 4511 |
| | | | | [Sorghum bicolor] | | | | |
| | | | | &gt:gi\|241917721\|gb\|EER90865.1\| | | | | |
| | | | | hypothetical protein SORBIDRAFT_01g007880 | | | | |
| | | | | [Sorghum bicolor] | | | | |
| | | ACL82964 | 223890398 | glutathione transporter [Zea mays] | 0.973226 | Zea mays | 3146 | 4512 |
| | | AAQ91200 | 37362135 | putative glutathione transporter | 0.943775 | Zea mays | 3147 | 4513 |
| | | | | [Zea mays] | | | | |
| | | BAK07444 | 326520370 | predicted protein | 0.907631 | Hordeum vulgare subsp. vulgare | 3148 | 4514 |
| | | | | [Hordeum vulgare subsp. vulgare] | | | | |
| | | NP_001051290 | 115455378 | Os03g0751100 [Oryza sativa Japonica Group] | 0.89826 | Oryza sativa Japonica Group | 3149 | 4515 |
| | | | | &gt:gi\|40539006\|gb\|AAR87263.1\| | | | | |
| | | | | putative oligopeptide transporter protein | | | | |
| | | | | [Oryza sativa Japonica Group] | | | | |
| | | | | &gt:gi\|108711110\|gb\|ABF98905.1\| | | | | |
| | | | | oligopeptide transporter 3, putative, expressed | | | | |
| | | | | [Oryza sativa Japonica Group] | | | | |
| | | | | &gt:gi\|113549761\|dbj\|BAF13204.1\| | | | | |
| | | | | Os03g0751100 [Oryza sativa Japonica Group] | | | | |
| | | | | &gt:gi\|218193762\|gb\|EEC76189.1\| | | | | |
| | | | | hypothetical protein OsI_13528 | | | | |
| | | | | [Oryza sativa Indica Group] | | | | |
| | | | | &gt:gi\|222625807\|gb\|EEE59939.1\| | | | | |
| | | | | hypothetical protein OsJ_12589 | | | | |
| | | | | [Oryza sativa Japonica Group] | | | | |
| | | ABF70152 | 102140012 | oligopeptide transporter OPT | 0.8166 | Musa acuminata | 3150 | 4516 |
| | | | | family protein [Musa acuminata] | | | | |
| | | AAO32313 | 21070919 | putative oligopeptide transporter protein | 0.854083 | Oryza sativa Japonica Group | 3151 | 4517 |
| | | | | [Oryza sativa Japonica Group] | | | | |
| | | O23482 | | RecName: Full = Oligopeptide transporter 3; | 0.801874 | Arabidopsis thaliana | 3152 | |
| | | | | Short = AtOPT3 | | | | |
| | | | | &gt:gi\|25083021\|gb\|AAN72034.1\| | | | | |
| | | | | isp4 like protein | | | | |
| | | | | [Arabidopsis thaliana] | | | | |
| | | AAK96781 | 15451019 | Unknown protein [Arabidopsis thaliana] | 0.800535 | Arabidopsis thaliana | 3153 | 4518 |
| | | XP_002868139 | 297800509 | hypothetical protein ARALYDRAFT_355122 | 0.801874 | Arabidopsis lyrata subsp. lyrata | 3154 | 4519 |
| | | | | [Arabidopsis lyrata subsp. lyrata] | | | | |
| | | | | &gt:gi\|297313975\|gb\|EFH44398.1\| | | | | |
| | | | | hypothetical protein ARALYDRAFT_355122 | | | | |
| | | | | [Arabidopsis lyrata subsp. lyrata] | | | | |
| | 1285-1304 | ACR35065 | 238008059 | unknown [Zea mays] | 1 | Zea mays | 3155 | 4520 |
| | 542-561 | ACN29008 | 223949848 | unknown [Zea mays] | 1 | Zea mays | 3156 | 4521 |
| | | NP_001147904 | 226502317 | serine carboxypeptidase 1 [Zea mays] | 0.996183 | Zea mays | 3157 | 4522 |
| | | | | &gt:gi\|195614482\|gb\|ACG29071.1\| | | | | |
| | | | | serine carboxypeptidase 1 precursor | | | | |
| | | | | [Zea mays] | | | | |
| | | XP_002463311 | 242051133 | hypothetical protein SORBIDRAFT_02g041610 | 0.902672 | Sorghum bicolor | 3158 | 4523 |
| | | | | [Sorghum bicolor] | | | | |
| | | | | &gt:gi\|241926688\|gb\|EER99832.1\| | | | | |
| | | | | hypothetical protein SORBIDRAFT_02g041610 | | | | |
| | | | | [Sorghum bicolor] | | | | |
| | | ACN26414 | 223944660 | unknown [Zea mays] | 0.877863 | Zea mays | 3159 | 4524 |
| | | NP_001147829 | 226509933 | LOC100281439 [Zea mays] | 0.874046 | Zea mays | 3160 | 4525 |
| | | | | &gt:gi\|195613988\|gb\|ACG28824.1\| | | | | |
| | | | | serine carboxypeptidase 1 precursor | | | | |
| | | | | [Zea mays] | | | | |
| | | BAC16131 | 50510131 | putative serine carboxypeptidase II-3 | 0.736641 | Oryza sativa Japonica Group | 3161 | 4526 |
| | | | | precursor [Oryza sativa Japonica Group] | | | | |
| | | BAJ94105 | 326490062 | predicted protein | 0.732824 | Hordeum vulgare subsp. vulgare | 3162 | 4527 |
| | | | | [Hordeum vulgare subsp. vulgare] | | | | |
| | | P52711 | | RecName: Full = Serine carboxypeptidase II-3; | 0.730916 | Hordeum vulgare subsp. vulgare | 3163 | |
| | | | | AltName: Full = CP-MIL3; Contains: | | | | |
| | | | | RecName: Full = Serine carboxypeptidase II-3 | | | | |
| | | | | chain A; Contains: | | | | |
| | | | | RecName: Full = Serine carboxypeptidase | | | | |
| | | | | II-3 chain B; Flags: Precursor | | | | |
| | | | | &gt:gi\|474392\|emb\|CAA55478.1\| | | | | |
| | | | | serine carboxylase II-3 | | | | |
| | | | | [Hordeum vulgare subsp. vulgare] | | | | |
| | | | | &gt:gi\|619350\|gb\|AAB31589.1\| | | | | |
| | | | | CP-MII.3 = serine carboxypeptidase | | | | |
| | | | | [Hordeum vulgare= barley, cv. Alexis, | | | | |
| | | | | aleurone, Peptide, 516 aa] | | | | |
| Predicted | 161-182 | NP_001044720 | 115440880 | Os01g0834500 [Oryza sativa Japonica Group] | 1 | Oryza sativa Japonica Group | 3164 | 4528 |
| siRNA | | | | &gt:gi\|115456215\|ref\|NP_001051708.1\| | | | | |
| 60718 | | | | Os03g0818400 [Oryza sativa Japonica Group] | | | | |
| | | | | &gt:gi\|297720551\|ref\|NP_001172637.1\| | | | | |
| | | | | Os01g0834601 [Oryza sativa Japonica Group] | | | | |
| | | | | &gt:gi\|313103637\|pdb\|3IZ6\|L Chain L, | | | | |
| | | | | Localization Of The Small Subunit | | | | |
| | | | | Ribosomal Proteins Into A 5.5 A Cryo-Em | | | | |
| | | | | Map Of Triticum Aestivum Translating 80s Ribosome | | | | |
| | | | | &gt:gi\|20805266\|dbj\|BAB92932.1\| | | | | |
| | | | | putative 40s ribosomal protein S23 | | | | |
| | | | | [Oryza sativa Japonica Group] | | | | |
| | | | | &gt:gi\|20805267\|dbj\|BAB92933.1\| | | | | |
| | | | | putative 40s ribosomal protein S23 | | | | |
| | | | | [Oryza sativa Japonica Group] | | | | |
| | | | | &gt:gi\|21671347\|dbj\|BAC02683.1\| | | | | |
| | | | | putative 40s ribosomal protein S23 | | | | |
| | | | | [Oryza sativa Japonica Group] | | | | |
| | | | | &gt:gi\|21671348\|dbj\|BAC02684.1\| | | | | |
| | | | | putative 40s ribosomal protein S23 | | | | |
| | | | | [Oryza sativa Japonica Group] | | | | |
| | | | | &gt:gi\|28876025\|gb\|AAO60034.1\| | | | | |
| | | | | 40S ribosomal protein S23 | | | | |
| | | | | [Oryza sativa Japonica Group] | | | | |
| | | | | &gt:gi\|29124115\|gb\|AAO65856.1\| | | | | |
| | | | | 40S ribosomal protein S23 | | | | |
| | | | | [Oryza sativa Japonica Group] | | | | |
| | | | | &gt:gi\|108711771\|gb\|ABF99566.1\| | | | | |
| | | | | 40S ribosomal protein S23, putative, expressed | | | | |
| | | | | [Oryza sativa Japonica Group] | | | | |
| | | | | &gt:gi\|113534251\|dbj\|BAF06634.1\| | | | | |
| | | | | Os01g0834500 [Oryza sativa Japonica Group] | | | | |
| | | | | &gt:gi\|113550179\|dbj\|BAF13622.1\| | | | | |
| | | | | Os03g0818400 [Oryza sativa Japonica Group] | | | | |
| | | | | &gt:gi\|125528286\|gb\|EAY76400.1\| | | | | |
| | | | | hypothetical protein OsI_04329 | | | | |
| | | | | [Oryza sativa Indica Group] | | | | |
| | | | | &gt:gi\|125546216\|gb\|EAY92355.1\| | | | | |
| | | | | hypothetical protein OsI_14082 | | | | |
| | | | | [Oryza sativa Indica Group] | | | | |
| | | | | &gt:gi\|215697420\|dbj\|BAG91414.1\| | | | | |
| | | | | unnamed protein product | | | | |
| | | | | [Oryza sativa Japonica Group] | | | | |
| | | | | &gt:gi\|215734943\|dbj\|BAG95665.1\| | | | | |
| | | | | unnamed protein product | | | | |
| | | | | [Oryza sativa Japonica Group] | | | | |
| | | | | &gt:gi\|255673847\|dbj\|BAH91367.1\| | | | | |
| | | | | Os01g0834601 [Oryza sativa Japonica Group] | | | | |
| | | | | &gt:gi\|326501134\|dbj\|BAJ98798.1\| | | | | |
| | | | | predicted protein | | | | |
| | | | | [Hordeum vulgare subsp. vulgare] | | | | |
| | | | | &gt:gi\|326506086\|dbj\|BAJ91282.1\| | | | | |
| | | | | predicted protein | | | | |
| | | | | [Hordeum vulgare subsp. vulgare] | | | | |
| | | NP_001131287 | 212722729 | hypothetical protein LOC100192600 | 0.992958 | Zea mays | 3165 | 4529 |
| | | | | [Zea mays] | | | | |
| | | | | &gt:gi\|242032479\|ref\|XP_002463634.1\| | | | | |
| | | | | hypothetical protein SORBIDRAFT_01g003410 | | | | |
| | | | | [Sorghum bicolor] | | | | |
| | | | | &gt:gi\|242059153\|ref\|XP_002458722.1\| | | | | |
| | | | | hypothetical protein SORBIDRAFT_03g039010 | | | | |
| | | | | [Sorghum bicolor] | | | | |
| | | | | &gt:gi\|242090801\|ref\|XP_002441233.1\| | | | | |
| | | | | hypothetical protein SORBIDRAFT_09g022840 | | | | |
| | | | | [Sorghum bicolor] | | | | |
| | | | | &gt:gi\|194691088\|gb\|ACF79628.1\| | | | | |
| | | | | unknown [Zea mays] | | | | |
| | | | | &gt:gi\|194697612\|gb\|ACF82890.1\| | | | | |
| | | | | unknown [Zea mays] | | | | |
| | | | | &gt:gi\|194702740\|gb\|ACF85454.1\| | | | | |
| | | | | unknown [Zea mays] | | | | |
| | | | | &gt:gi\|195606082\|gb\|ACG24871.1\| | | | | |
| | | | | 40S ribosomal protein S23 | | | | |
| | | | | [Zea mays] | | | | |
| | | | | &gt:gi\|195618728\|gb\|ACG31194.1\| | | | | |
| | | | | 40S ribosomal protein S23 | | | | |
| | | | | [Zea mays] | | | | |
| | | | | &gt:gi\|195619636\|gb\|ACG31648.1\| | | | | |
| | | | | 40S ribosomal protein S23 | | | | |
| | | | | [Zea mays] | | | | |
| | | | | &gt:gi\|195625318\|gb\|ACG34489.1\| | | | | |
| | | | | 40S ribosomal protein S23 | | | | |
| | | | | [Zea mays] | | | | |
| | | | | &gt:gi\|195628702\|gb\|ACG36181.1\| | | | | |
| | | | | 40S ribosomal protein S23 | | | | |
| | | | | [Zea mays] | | | | |
| | | | | &gt:gi\|195657679\|gb\|ACG48307.1\| | | | | |
| | | | | 40S ribosomal protein S23 | | | | |
| | | | | [Zea mays] | | | | |
| | | | | &gt:gi\|238012290\|gb\|ACR37180.1\| | | | | |
| | | | | unknown [Zea mays] | | | | |
| | | | | &gt:gi\|241917488\|gb\|EER90632.1\| | | | | |
| | | | | hypothetical protein SORBIDRAFT_01g003410 | | | | |
| | | | | [Sorghum bicolor] | | | | |
| | | | | &gt:gi\|241930697\|gb\|EES03842.1\| | | | | |
| | | | | hypothetical protein SORBIDRAFT_03g039010 | | | | |
| | | | | [Sorghum bicolor] | | | | |
| | | | | &gt:gi\|241946518\|gb\|EES19663.1\| | | | | |
| | | | | hypothetical protein SORBIDRAFT_09g022840 | | | | |
| | | | | [Sorghum bicolor] | | | | |
| | | ACG32843 | 195622025 | 40S ribosomal protein S23 | 0. 985915 | Zea mays | 3166 | 4530 |
| | | | | [Zea mays] | | | | |
| | | ACF06518 | 192910819 | 40S ribosomal protein S23 | 0.978873 | Elaeis guineensis | 3167 | 4531 |
| | | | | [Elaeis guineensis] | | | | |
| | | | | &gt:gi\|192910894\|gb\|ACF06555.1\| | | | | |
| | | | | 40S ribosomal protein S23 | | | | |
| | | | | [Elaeis guineensis] | | | | |
| | | ACF06519 | 192910821 | 40S ribosomal protein S23 | 0.971831 | Elaeis guineensis | 3168 | 4532 |
| | | | | [Elaeis guineensis] | | | | |
| | | ABBl6993 | 77999292 | unknown [Solanum tuberosum] | 0.964789 | Solanum tuberosum | 3169 | 4533 |
| | | XP_002523902 | 255565825 | 40S ribosomal protein S23, putative | 0.964789 | Ricinus communis | 3170 | 4534 |
| | | | | [Ricinus communis] | | | | |
| | | | | &gt:gi\|255568414\|ref\|XP_002525181.1\| | | | | |
| | | | | 40S ribosomal protein S23, putative | | | | |
| | | | | [Ricinus communis] | | | | |
| | | | | &gt:gi\|223535478\|gb\|EEF37147.1\| | | | | |
| | | | | 40S ribosomal protein S23, putative | | | | |
| | | | | [Ricinus communis] | | | | |
| | | | | &gt:gi\|223536832\|gb\|EEF38471.1\| | | | | |
| | | | | 40S ribosomal protein S23, putative | | | | |
| | | | | [Ricinus communis] | | | | |
| | | XP_002279025 | 225439887 | PREDICTED: hypothetical protein | 0.964789 | Vitis vinifera | 3171 | 4535 |
| | | | | [Vitis vinifera] | | | | |
| Predicted | 658-679 | NP_001131832 | 212724035 | hypothetical protein LOC100193207 | 1 | Zea mays | 3172 | 4536 |
| siRNA | | | | [Zea mays] | | | | |
| 60742 | | | | &gt:gi\|194692668\|gb\|ACF80418.1\| | | | | |
| | | | | unknown [Zea mays] | | | | |
| | | XP_002455284 | 242052276 | hypothetical protein SORBIDRAFT_03g007780 | 0.745562 | Sorghum bicolor | 3173 | 4537 |
| | | | | [Sorghum bicolor] | | | | |
| | | | | &gt:gi\|241927259\|gb\|EES00404.1\| | | | | |
| | | | | hypothetical protein SORBIDRAFT_03g007780 | | | | |
| | | | | [Sorghum bicolor] | | | | |
| | 1293-1314 | XP_002451722 | 242060865 | hypothetical protein SORBIDRAFT_04g006610 | 1 | Sorghum bicolor | 3174 | 4538 |
| | | | | [Sorghum bicolor] | | | | |
| | | | | &gt:gi\|241931553\|gb\|EES04698.1\| | | | | |
| | | | | hypothetical protein SORBIDRAFT_04g006610 | | | | |
| | | | | [Sorghum bicolor] | | | | |
| Predicted | 437-454 | NP_001145763 | 226491569 | hypothetical protein LOC100279270 | 1 | Zea mays | 3175 | 4539 |
| siRNA | | | | [Zea mays] | | | | |
| 60833 | | | | &gt:gi\|219884277\|gb\|ACL52513.1\| | | | | |
| | | | | unknown [Zea mays] | | | | |
| | | | | &gt:gi\|219884335\|gb\|ACL52542.1\| | | | | |
| | | | | unknown [Zea mays] | | | | |
| | | | | &gt:gi\|224028501\|gb\|ACN33326.1\| | | | | |
| | | | | unknown [Zea mays] | | | | |
| | | NP_001054556 | 115461912 | Os05g0132100 [Oryza sativa Japonica Group] | 0.828358 | Oryza sativa Japonica Group | 3176 | 4540 |
| | | | | &gt:gi\|113578107\|dbj\|BAF16470.1\| | | | | |
| | | | | Os05g0132100 [Oryza sativa Japonica Group] | | | | |
| | | | | &gt:gi\|222630090\|gb\|EEE62222.1\| | | | | |
| | | | | hypothetical protein OsJ_17009 | | | | |
| | | | | [Oryza sativa Japonica Group] | | | | |
| | | EEC78466 | 54362548 | hypothetical protein OsI_18335 | 0.810448 | Oryza sativa Indica Group | 3177 | |
| | | | | [Oryza sativa Indica Group] | | | | |
| | | BAG97315 | 32990827 | unnamed protein product | 0.753731 | Oryza sativa Japonica Group | 3178 | 4541 |
| | | | | [Oryza sativa Japonica Group] | | | | |
| | 1660-1677 | XP_002456424 | 242054556 | hypothetical protein SORBIDRAFT_03g036030 | 1 | Sorghum bicolor | 3179 | 4542 |
| | | | | [Sorghum bicolor] | | | | |
| | | | | &gt:gi\|241928399\|gb\|EES01544.1\| | | | | |
| | | | | hypothetical protein SORBIDRAFT_03g036030 | | | | |
| | | | | [Sorghum bicolor] | | | | |
| | | NP_001142208 | 226496106 | hypothetical protein LOC100274376 | 0.987903 | Zea mays | 3180 | 4543 |
| | | | | [Zea mays] | | | | |
| | | | | &gt:gi\|194702286\|gb\|ACF85227.1\| | | | | |
| | | | | unknown [Zea mays] | | | | |
| | | | | &gt:gi\|194707600\|gb\|ACF87884.1\| | | | | |
| | | | | unknown [Zea mays] | | | | |
| | | NP_001044395 | 115440230 | Os01g0772800 [Oryza sativa Japonica Group] | 0.947581 | Oryza sativa Japonica Group | 3181 | 4544 |
| | | | | &gt:gi\|20160917\|dbj\|BAB89854.1\| | | | | |
| | | | | putative signal recognition particle 54 kD protein | | | | |
| | | | | [Oryza sativa Japonica Group] | | | | |
| | | | | &gt:gi\|21743252\|dbj\|BAC03250.1\| | | | | |
| | | | | putative signal recognition particle 54 kD protein | | | | |
| | | | | [Oryza sativa Japonica Group] | | | | |
| | | | | &gt:gi\|32879776\|dbj\|BAC79360.1\| | | | | |
| | | | | signal recognition particle 54 kDa subunit | | | | |
| | | | | [Oryza sativa Japonica Group] | | | | |
| | | | | &gt:gi\|113533926\|dbj\|BAF06309.1\| | | | | |
| | | | | Os01g0772800 [Oryza sativa Japonica Group] | | | | |
| | | | | &gt:gi\|215767979\|dbj\|BAH00208.1\| | | | | |
| | | | | unnamed protein product | | | | |
| | | | | [Oryza sativa Japonica Group] | | | | |
| | | | | &gt:gi\|222619331\|gb\|EEE55463.1\| | | | | |
| | | | | hypothetical protein OsJ_03626 | | | | |
| | | | | [Oryza sativa Japonica Group] | | | | |
| | | EEC71559 | 54362548 | hypothetical protein OsI_03915 | 0.945565 | Oryza sativa Indica Group | 3182 | |
| | | | | [Oryza sativa Indica Group] | | | | |
| | | XP_002517663 | 255553240 | signal recognition particle 54 kD protein, putative | 0.905242 | Ricinus communis | 3183 | 4545 |
| | | | | [Ricinus communis] | | | | |
| | | | | &gt:gi\|223543295\|gb\|EEF44827.1\| | | | | |
| | | | | signal recognition particle 54 kD protein, putative | | | | |
| | | | | [Ricinus communis] | | | | |
| | | XP_002299799 | 224059269 | predicted protein [Populus trichocarpa] | 0.887097 | Populus trichocarpa | 3184 | 4546 |
| | | | | &gt:gi\|222847057\|gb\|EEE84604.1\| | | | | |
| | | | | predicted protein [Populus trichocarpa] | | | | |
| | | XP_002264159 | 225442909 | PREDICTED: hypothetical protein | 0.887097 | Vitis vinifera | 3185 | 4547 |
| | | | | [Vitis vinifera] | | | | |
| | | XP_002322968 | 224139051 | predicted protein [Populus trichocarpa] | 0.893145 | Populus trichocarpa | 3186 | 4548 |
| | | | | &gt:gi\|222867598\|gb\|EEF04729.1\| | | | | |
| | | | | predicted protein [Populus trichocarpa] | | | | |
| | | ABK95819 | 118487995 | unknown [Populus trichocarpa] | 0.885081 | Populus trichocarpa | 3187 | 4549 |
| | 1015-1032 | XP_002466942 | 242039094 | hypothetical protein SORBIDRAFT_01g017160 | 1 | Sorghum bicolor | 3188 | 4550 |
| | | | | [Sorghum bicolor] | | | | |
| | | | | &gt:gi\|241920796\|gb\|EER93940.1\| | | | | |
| | | | | hypothetical protein SORBIDRAFT_01g017160 | | | | |
| | | | | [Sorghum bicolor] | | | | |
| | | NP_001146067 | 226501573 | hypothetical protein LOC100279598 | 0.925144 | Zea mays | 3189 | 4551 |
| | | | | [Zea mays] | | | | |
| | | | | &gt:gi\|224030981\|gb\|ACN34566.1\| | | | | |
| | | | | unknown [Zea mays] | | | | |
| | | ACG47544 | 195656152 | transposon protein [Zea mays] | 0.932821 | Zea mays | 3190 | 4552 |
| | | XP_002466943 | 242039096 | hypothetical protein SORBIDRAFT_01g017170 | 0.815739 | Sorghum bicolor | 3191 | 4553 |
| | | | | [Sorghum bicolor] | | | | |
| | | | | &gt:gi\|241920797\|gb\|EER93941.1\| | | | | |
| | | | | hypothetical protein SORBIDRAFT_01g017170 | | | | |
| | | | | [Sorghum bicolor] | | | | |
| | | BAJ94552 | 326494865 | predicted protein | 0.761996 | Hordeum vulgare subsp. vulgare | 3192 | 4554 |
| | | | | [Hordeum vulgare subsp. vulgare] | | | | |
| | | NP_001065056 | 115482925 | Os10g0515200 [Oryza sativa Japonica Group] | 0.740883 | Oryza sativa Japonica Group | 3193 | 4555 |
| | | | | &gt:gi\|10140669\|gb\|AAG13504.1\|AC068924_9 | | | | |
| | | | | putative cytochrome P450 | | | | |
| | | | | [Oryza sativa Japonica Group] | | | | |
| | | | | &gt:gi\|31433051\|gb\|AAP54611.1\| | | | | |
| | | | | transposon protein, putative, unclassified, expressed | | | | |
| | | | | [Oryza sativa Japonica Group] | | | | |
| | | | | &gt:gi\|113639665\|dbj\|BAF26970.1\| | | | | |
| | | | | Os10g0515200 [Oryza sativa Japonica Group] | | | | |
| | | | | &gt:gi\|215740677\|dbj\|BAG97333.1\| | | | | |
| | | | | unnamed protein product | | | | |
| | | | | [Oryza sativa Japonica Group] | | | | |
| | | EAY79199 | 54362548 | hypothetical protein OsI_34311 | 0.735125 | Oryza sativa Indica Group | 3194 | |
| | | | | [Oryza sativa Indica Group] | | | | |
| | | BAK03964 | 326520078 | predicted protein | 0.737044 | Hordeum vulgare subsp. vulgare | 3195 | 4556 |
| | | | | [Hordeum vulgare subsp. vulgare] | | | | |
| | | BAJ93808 | 326488278 | predicted protein | 0.723608 | Hordeum vulgare subsp. vulgare | 3196 | 4557 |
| | | | | [Hordeum vulgare subsp. vulgare] | | | | |
| | 14111-428 | XP_002448742 | 242077611 | hypothetical protein SORBIDRAFT_06g032450 | 1 | Sorghum bicolor | 3197 | 4558 |
| | | | | [Sorghum bicolor] | | | | |
| | | | | &gt:gi\|241939925\|gb\|EES13070.1\| | | | | |
| | | | | hypothetical protein SORBIDRAFT_06g032450 | | | | |
| | | | | [Sorghum bicolor] | | | | |
| | | ACF78749 | 238908545 | unknown [Zea mays] | 0.969199 | Zea mays | 3198 | 4559 |
| | | | | &gt:gi\|194707726\|gb\|ACF87947.1\| | | | | |
| | | | | unknown [Zea mays] | | | | |
| | | | | &gt:gi\|195611472\|gb\|ACG27566.1\| | | | | |
| | | | | lysine-specific histone demethylase 1 | | | | |
| | | | | [Zea mays] | | | | |
| | | | | &gt:gi\|195616900\|gb\|ACG30280.1\| | | | | |
| | | | | lysine-specific histone demethylase 1 | | | | |
| | | | | [Zea mays] | | | | |
| | | | | &gt:gi\|223950041\|gb\|ACN29104.1\| | | | | |
| | | | | unknown [Zea mays] | | | | |
| | | | | &gt:gi\|224031369\|gb\|ACN34760.1\| | | | | |
| | | | | unknown [Zea mays] | | | | |
| | | NP_001054218 | 115461235 | Os04g0671200 [Oryza sativa Japonica Group] | 0.905544 | Oryza sativa Japonica Group | 3199 | 4560 |
| | | | | &gt:gi\|32488409\|emb\|CAE02834.1\| | | | | |
| | | | | OSJNBa0043A12.39 [Oryza sativa Japonica Group] | | | | |
| | | | | &gt:gi\|90265248\|emb\|CAH67701.1\| | | | | |
| | | | | H0624F09.9 [Oryza sativa Indica Group] | | | | |
| | | | | &gt:gi\|113565789\|dbj\|BAF16132.1\| | | | | |
| | | | | Os04g0671200 [Oryza sativa Japonica Group] | | | | |
| | | | | &gt:gi\|125550177\|gb\|EAY95999.1\| | | | | |
| | | | | hypothetical protein OsI_17870 | | | | |
| | | | | [Oryza sativa Indica Group] | | | | |
| | | | | &gt:gi\|125592017\|gb\|EAZ32367.1\| | | | | |
| | | | | hypothetical protein OsJ_16578 | | | | |
| | | | | [Oryza sativa Japonica Group] | | | | |
| | | XP_002448744 | 242077615 | hypothetical protein SORBIDRAFT_06g032460 | 0.7577 | Sorghum bicolor | 3200 | 4561 |
| | | | | [Sorghum bicolor] | | | | |
| | | | | &gt:gi\|241939927\|gb\|EES13072.1\| | | | | |
| | | | | hypothetical protein SORBIDRAFT_06g032460 | | | | |
| | | | | [Sorghum bicolor] | | | | |
| | | NP_001054219 | 115461237 | Os04g0671300 [Oryza sativa Japonica Group] | 0.75154 | Oryza sativa Japonica Group | 3201 | 4562 |
| | | | | &gt:gi\|90265249\|emb\|CAH67702.1\| | | | | |
| | | | | H0624F09.10 [Oryza sativa Indica Group] | | | | |
| | | | | &gt:gi\|113565790\|dbj\|BAF16133.1\| | | | | |
| | | | | Os04g0671300 [Oryza sativa Japonica Group] | | | | |
| | | | | &gt:gi\|215704120\|dbj\|BAG92960.1\| | | | | |
| | | | | unnamed protein product | | | | |
| | | | | [Oryza sativa Japonica Group] | | | | |
| | | | | &gt:gi\|218195801\|gb\|EEC78228.1\| | | | | |
| | | | | hypothetical protein OsI_17871 | | | | |
| | | | | [Oryza sativa Indica Group] | | | | |
| | | | | &gt:gi\|222629752\|gb\|EEE61884.1\| | | | | |
| | | | | hypothetical protein OsJ_16579 | | | | |
| | | | | [Oryza sativa Japonica Group] | | | | |
| | | CAE03599 | 70663936 | OSJNBb0004A17.1 [Oryza sativa Japonica Group] | 0.74538 | Oryza sativa Japonica Group | 3202 | 4563 |
| | | NP_001170164 | 293332860 | hypothetical protein LOC100384102 | 0.743326 | Zea mays | 3203 | 4564 |
| | | | | [Zea mays] | | | | |
| | | | | &gt:gi\|195613858\|gb\|ACG28759.1\| | | | | |
| | | | | lysine-specific histone demethylase 1 | | | | |
| | | | | [Zea mays] | | | | |
| | | ACG29077 | 195614493 | lysine-specific histone demethylase 1 | 0.73922 | Zea mays | 3204 | 4565 |
| | | | | [Zea mays] | | | | |
| | | ACN36050 | 224033948 | unknown [Zea mays] | 0.741273 | Zea mays | 3205 | 4566 |
| | 1657-1674 | NP_001183903 | 308080099 | hypothetical protein LOC100502496 | 1 | Zea mays | 3206 | 4567 |
| | | | | [Zea mays] | | | | |
| | | | | &gt:gi\|238015356\|gb\|ACR38713.1\| | | | | |
| | | | | unknown [Zea mays] | | | | |
| | 462-479 | XP_002467261 | 242039732 | hypothetical protein SORBIDRAFT_01g022230 | 1 | Sorghum bicolor | 3207 | 4568 |
| | | | | [Sorghum bicolor] | | | | |
| | | | | &gt:gi\|24192115\|gb\|EER94259.1\| | | | | |
| | | | | hypothetical protein SORBIDRAFT_01g022230 | | | | |
| | | | | [Sorghum bicolor] | | | | |
| | | NP_001150190 | 226490935 | PP2Ac-4 - Phosphatase 2A isoform 4 belonging | 0.984127 | Zea mays | 3208 | 4569 |
| | | | | to family 2 [Zea mays] | | | | |
| | | | | &gt:gi\|195637426\|gb\|ACG38181.1\| | | | | |
| | | | | PP2Ac-4 -Phosphatase 2A isoform 4 belonging | | | | |
| | | | | to family 2 [Zea mays] | | | | |
| | | XP_002465801 | 242036812 | hypothetical protein SORBIDRAFT_01g046020 | 0.942857 | Sorghum bicolor | 3209 | 4570 |
| | | | | [Sorghum bicolor] | | | | |
| | | | | &gt:gi\|241919655\|gb\|EER92799.1\| | | | | |
| | | | | hypothetical protein SORBIDRAFT_01g046020 | | | | |
| | | | | [Sorghum bicolor] | | | | |
| | | ACG34764 | 195625867 | PP2Ac-5 - Phosphatase 2A isoform 5 belonging | 0.939683 | Zea mays | 3210 | 4571 |
| | | | | to family 2 [Zea mays] | | | | |
| | | P0C5D7 | | RecName: Full = Putative serine/threonine-protein | 0.942857 | Oryza sativa Indica Group | 3211 | |
| | | | | phosphatase PP2A-4 catalytic subunit | | | | |
| | | | | &gt:gi\|5714762\|gb\|AAD48068.1\|AF173881_1 | | | | |
| | | | | serine/threonine protein phosphatase PP2A-4 catalytic subunit | | | | |
| | | | | [Oryza sativa Indica Group] | | | | |
| | | NP_001130096 | 212274514 | hypothetical protein LOC100191189 | 0.936508 | Zea mays | 3212 | 4572 |
| | | | | [Zea mays] | | | | |
| | | | | &gt:gi\|194688278\|gb\|ACF78223.1\| | | | | |
| | | | | unknown [Zea mays] | | | | |
| | | ABOl6371 | 126517971 | serine/threonine-protein phosphatase PP2A-1 catalytic subunit | 0.936508 | Triticum aestivum | 3213 | 4573 |
| | | | | [Triticum aestivum] | | | | |
| | | NP_001149212 | 226495576 | PP2Ac-5 - Phosphatase 2A isoform 5 belonging | 0.933333 | Zea mays | 3214 | 4574 |
| | | | | to family 2 [Zea mays] | | | | |
| | | | | &gt:gi\|195625500\|gb\|ACG34580.1\| | | | | |
| | | | | PP2Ac-5 - Phosphatase 2A isoform 5 belonging | | | | |
| | | | | to family 2 [Zea mays] | | | | |
| | | NP_001049084 | 115450966 | Os03g0167700 [Oryza sativa Japonica Group] | 0.92381 | Oryza sativa Japonica Group | 3215 | 4575 |
| | | | | &gt:gi\|108706380\|gb\|ABF94175.1\| | | | | |
| | | | | Serine/threonine protein phosphatase | | | | |
| | | | | PP2A-4 catalytic subunit, putative, expressed | | | | |
| | | | | [Oryza sativa Japonica Group] | | | | |
| | | | | &gt:gi\|113547555\|dbj\|BAF10998.1\| | | | | |
| | | | | Os03g0167700 [Oryza sativa Japonica Group] | | | | |
| | | | | &gt:gi\|215697662\|dbj\|BAG91656.1\| | | | | |
| | | | | unnamed protein product | | | | |
| | | | | [Oryza sativa Japonica Group] | | | | |
| | | | | &gt:gi\|215701359\|dbj\|BAG92783.1\| | | | | |
| | | | | unnamed protein product | | | | |
| | | | | [Oryza sativa Japonica Group] | | | | |
| | 1228-1245 | AAL75481 | 46200524 | putative TNP2 [Zea mays] | 1 | Zea mays | 3216 | 4576 |
| | 735-752 | NP_001130504 | 212275861 | hypothetical protein LOC100191603 | 1 | Zea mays | 3217 | 4577 |
| | | | | [Zea mays] | | | | |
| | | | | &gt:gi\|195616620\|gb\|ACG30140.1\| | | | | |
| | | | | lysine-specific histone demethylase 1 | | | | |
| | | | | [Zea mays] | | | | |
| | 114-131 | XP_002447908 | 242075943 | hypothetical protein SORBIDRAFT_06g017770 | 1 | Sorghum bicolor | 3218 | 4578 |
| | | | | [Sorghum bicolor] | | | | |
| | | | | &gt:gi\|241939091\|gb\|EES12236.1\| | | | | |
| | | | | hypothetical protein SORBIDRAFT_06g017770 | | | | |
| | | | | [Sorghum bicolor] | | | | |
| | | NP_001105527 | 162461261 | barley mlo defense gene homolog3 [Zea mays] | 0.916667 | Zea mays | 3219 | 4579 |
| | | | | &gt:gi\|13784979\|gb\|AAK38339.1\| | | | | |
| | | | | seven transmembrane protein Mlo3 | | | | |
| | | | | [Zea mays] | | | | |
| | | EAY94273 | 54362548 | hypothetical protein OsI_16042 | 0.702381 | Oryza sativa Indica Group | 3220 | |
| | | | | [Oryza sativa Indica Group] | | | | |
| | | CAD40974 | 32482917 | OSJNBa0027P08.3 [Oryza sativa Japonica Group] | 0.702381 | Oryza sativa Japonica Group | 3221 | 4580 |
| | | | | &gt:gi\|21742880\|emb\|CAD41046.1\| | | | | |
| | | | | OSJNBa0058G03.6 [Oryza sativa Japonica Group] | | | | |
| | | | | &gt:gi\|125590510\|gb\|EAZ30860.1\| | | | | |
| | | | | hypothetical protein OsJ_14932 | | | | |
| | | | | [Oryza sativa Japonica Group] | | | | |
| | 361-378 | XP_002442895 | 242084939 | hypothetical protein SORBIDRAFT_08g004520 | 1 | Sorghum bicolor | 3222 | 4581 |
| | | | | [Sorghum bicolor] | | | | |
| | | | | &gt:gi\|241943588\|gb\|EES16733.1\| | | | | |
| | | | | hypothetical protein SORBIDRAFT_08g004520 | | | | |
| | | | | [Sorghum bicolor] | | | | |
| | | ACN36368 | 224034584 | unknown [Zea mays] | 0.769231 | Zea mays | 3223 | 4582 |
| | | NP_001152351 | 226496368 | bile acid sodium symporter/transporter | 0.764268 | Zea mays | 3224 | 4583 |
| | | | | [Zea mays] | | | | |
| | | | | &gt:gi\|195655405\|gb\|ACG47170.1\| | | | | |
| | | | | bile acid sodium symporter/transporter | | | | |
| | | | | [Zea mays] | | | | |
| | | ABA96556 | 108862058 | Sodium Bile acid symporter family protein, expressed | 0.722084 | Oryza sativa Japonica Group | 3225 | 4584 |
| | | | | [Oryza sativa Japonica Group] | | | | |
| | 680-697 | NP_001048539 | 115449732 | Os02g0820000 [Oryza sativa Japonica Group] | 1 | Oryza sativa Japonica Group | 3226 | 4585 |
| | | | | &gt:gi\|113538070\|dbj\|BAF10453.1\| | | | | |
| | | | | Os02g0820000 [Oryza sativa Japonica Group] | | | | |
| | | AAK64283 | 14495345 | protein phosphatase [Oryza sativa] | 0.921283 | Oryza sativa | 3227 | 4586 |
| | | | | &gt:gi\|48716362\|dbj\|BAD22973.1\| protein | | | | |
| | | | | phosphatase [Oryza sativa Japonica Group] | | | | |
| | | | | &gt:gi\|48716497\|dbj\|BAD23102.1\| protein | | | | |
| | | | | phosphatase [Oryza sativa Japonica Group] | | | | |
| | | | | &gt:gi\|215767785\|dbj\|BAH00014.1\| unnamed protein | | | | |
| | | | | product [Oryza sativa Japonica Group] | | | | |
| | | | | &gt:gi\|218191835\|gb\|EEC74262.1\| | | | | |
| | | | | hypothetical protein OsI_09476 | | | | |
| | | | | [Oryza sativa Indica Group] | | | | |
| | | | | &gt:gi\|222623927\|gb\|EEE58059.1\| | | | | |
| | | | | hypothetical protein OsJ_08899 | | | | |
| | | | | [Oryza sativa Japonica Group] | | | | |
| | | NP_001105341 | 162462901 | serine/threonine-protein phosphatase | 0.868805 | Zea mays | 3228 | 4587 |
| | | | | PP1 [Zea mays] | | | | |
| | | | | &gt:gi\|130709\|sp\|P22198.1\|PP1_MAIZE | | | | |
| | | | | RecName: Full = Serine/threonine-protein phosphatase PP1 | | | | |
| | | | | &gt:gi\|168723\|gb\|AAA33545.1\| | | | | |
| | | | | protein phosphatase-1 [Zea mays] | | | | |
| | | | | &gt:gi\|223944929\|gb\|ACN26548.1\| | | | | |
| | | | | unknown [Zea mays] | | | | |
| | | | | &gt:gi\|445586\|prf\|\|1909338A | | | | |
| | | | | protein phosphatase 1 | | | | |
| | | XP_002521968 | 255561918 | serine/threonine protein phosphatase, putative | 0.784257 | Ricinus communis | 3229 | 4588 |
| | | | | [Ricinus communis] | | | | |
| | | | | &gt:gi\|223538772\|gb\|EEF40372.1\| | | | | |
| | | | | serine/threonine protein phosphatase, putative | | | | |
| | | | | [Ricinus communis] | | | | |
| | | XP_002265552 | 225453025 | PREDICTED: hypothetical protein | 0.781341 | Vitis vinifera | 3230 | 4589 |
| | | | | [Vitis vinifera] | | | | |
| | | CAN71489 | 147842159 | hypothetical protein VITISV_005340 | 0.781341 | Vitis vinifera | 3231 | |
| | | | | [Vitis vinifera] | | | | |
| | | ACU24610 | 255648313 | unknown [Glycine max] | 0.790087 | Glycine max | 3232 | 4590 |
| | | XP_002319103 | 224123531 | predicted protein [Populus trichocarpa] | 0.769679 | Populus trichocarpa | 3233 | 4591 |
| | | | | &gt:gi\|222857479\|gb\|EEE95026.1\| | | | | |
| | | | | predicted protein [Populus trichocarpa] | | | | |
| | 103-120 | NP_001168450 | 293331506 | hypothetical protein LOC100382223 | 1 | Zea mays | 3234 | 4592 |
| | | | | [Zea mays] | | | | |
| | | | | &gt:gi\|223948377\|gb\|ACN28272.1\| | | | | |
| | | | | unknown [Zea mays] | | | | |
| | | NP_001142379 | 226504555 | hypothetical protein LOC100274552 | 0.911688 | Zea mays | 3235 | 4593 |
| | | | | [Zea mays] | | | | |
| | | | | &gt:gi\|194708552\|gb\|ACF88360.1\| | | | | |
| | | | | unknown [Zea mays] | | | | |
| | | XP_002438780 | 242096579 | hypothetical protein SORBIDRAFT_10g026080 | 0.901299 | Sorghum bicolor | 3236 | 4594 |
| | | | | [Sorghum bicolor] | | | | |
| | | | | &gt:gi\|241917003\|gb\|EER90147.1\| | | | | |
| | | | | hypothetical protein SORBIDRAFT_10g026080 | | | | |
| | | | | [Sorghum bicolor] | | | | |
| | | BAJ93429 | 326528494 | predicted protein | 0.823377 | Hordeum vulgare subsp. vulgare | 3237 | 4595 |
| | | | | [Hordeum vulgare subsp. vulgare] | | | | |
| | | NP_001058250 | 115469301 | Os06g0655500 [Oryza sativa Japonica Group] | 0.820779 | Oryza sativa Japonica Group | 3238 | 4596 |
| | | | | &gt:gi\|51535613\|dbj\|BAD37556.1\| | | | | |
| | | | | kelch repeat containing F-box protein-like | | | | |
| | | | | [Oryza sativa Japonica Group] | | | | |
| | | | | &gt:gi\|51536380\|dbj\|BAD37573.1\| | | | | |
| | | | | kelch repeat containing F-box protein-like | | | | |
| | | | | [Oryza sativa Japonica Group] | | | | |
| | | | | &gt:gi\|113596290\|dbj\|BAF20164.1\| | | | | |
| | | | | Os06g0655500 [Oryza sativa Japonica Group] | | | | |
| | | | | &gt:gi\|215697408\|dbj\|BAG91402.1\| | | | | |
| | | | | unnamed protein product | | | | |
| | | | | [Oryza sativa Japonica Group] | | | | |
| | 698-715 | NP_001149387 | 226496780 | LOC100283013 [Zea mays] | 1 | Zea mays | 3239 | 4597 |
| | | | | &gt:gi\|195626870\|gb\|ACG35265.1\| serine/threonine- | | | | |
| | | | | protein phosphatase PP1 [Zea mays] | | | | |
| | 175-192 | ACG35262 | 195626863 | seed specific protein Bn15D17A [Zea mays] | 1 | Zea mays | 3240 | 4598 |
| | | NP_001148471 | 226528520 | seed specific protein Bn15D17A [Zea mays] | 0.98722 | Zea mays | 3241 | 4599 |
| | | | | &gt:gi\|195619590\|gb\|ACG31625.1\| | | | | |
| | | | | seed specific protein Bn15D17A [Zea mays] | | | | |
| | | XP_002441388 | 242091110 | hypothetical protein SORBIDRAFT_09g025710 | 0.817891 | Sorghum bicolor | 3242 | 4600 |
| | | | | [Sorghum bicolor] | | | | |
| | | | | &gt:gi\|241946673\|gb\|EES19818.1\| | | | | |
| | | | | hypothetical protein SORBIDRAFT_09g025710 | | | | |
| | | | | [Sorghum bicolor] | | | | |
| | 542-559 | NP_001105206 | 162459735 | SET domain protein SDG117 [Zea mays] | 1 | Zea mays | 3243 | 4601 |
| | | | | &gt:gi\|28261315\|gb\|AAO32935.1\| | | | | |
| | | | | SET domain protein SDG117 [Zea mays] | | | | |
| | 258-275 | NP_001064578 | 115481969 | Os10g0410600 [Oryza sativa Japonica Group] | 1 | Oryza sativa Japonica Group | 3244 | 4602 |
| | | | | &gt:gi\|158513707\|sp\|A3C4N5.2\|PP2A4_ORYSJ | | | | |
| | | | | RecName: Full = Serine/threonine-protein phosphatase | | | | |
| | | | | PP2A-4 catalytic subunit | | | | |
| | | | | &gt:gi\|78708615\|gb\|ABB47590.1\| | | | | |
| | | | | Serine/threonine protein phosphatase | | | | |
| | | | | PP2A-4 catalytic subunit, putative, expressed | | | | |
| | | | | [Oryza sativa Japonica Group] | | | | |
| | | | | &gt:gi\|113639187\|dbj\|BAF26492.1\| | | | | |
| | | | | Os10g0410600 [Oryza sativa Japonica Group] | | | | |
| | | | | &gt:gi\|215704585\|dbj\|BAG94218.1\| | | | | |
| | | | | unnamed protein product | | | | |
| | | | | [Oryza sativa Japonica Group] | | | | |
| | | | | &gt:gi\|222612811\|gb\|EEE50943.1\| | | | | |
| | | | | hypothetical protein OsJ_31490 | | | | |
| | | | | [Oryza sativa Japonica Group] | | | | |
| | 162-179 | BAJ85175 | 326493427 | predicted protein | 1 | Hordeum vulgare subsp. vulgare | 3245 | 4603 |
| | | | | [Hordeum vulgare subsp. vulgare] | | | | |
| | | | | &gt:gi\|326500666\|dbj\|BAJ94999.1\| | | | | |
| | | | | predicted protein | | | | |
| | | | | [Hordeum vulgare subsp. vulgare] | | | | |
| | | | | &gt:gi\|326513058\|dbj\|BAK03436.1\| | | | | |
| | | | | predicted protein | | | | |
| | | | | [Hordeum vulgare subsp. vulgare] | | | | |
| | | BAF31132 | 114213457 | catalytic subunit of protein | 0.8375 | Vicia faba | 3246 | 4604 |
| | | | | phosphatase 1 [Vicia faba] | | | | |
| | | NP_001049669 | 115452136 | Os03g0268000 [Oryza sativa Japonica Group] | 0.80625 | Oryza sativa Japonica Group | 3247 | 4605 |
| | | | | &gt:gi\|108935873\|sp\|P48489.2\|PP1_ORYSJ | | | | |
| | | | | RecName: Full = Serine/threonine-protein phosphatase | | | | |
| | | | | PP1 &gt:gi\|108707369\|gb\|ABF95164.1\| | | | | |
| | | | | Serine/threonine protein phosphatase PP1, putative, | | | | |
| | | | | expressed [Oryza sativa Japonica Group] | | | | |
| | | | | &gt:gi\|108707370\|gb\|ABF95165.1\| | | | | |
| | | | | Serine/threonine protein phosphatase PP1, putative, | | | | |
| | | | | expressed [Oryza sativa Japonica Group] | | | | |
| | | | | &gt:gi\|108707371\|gb\|ABF95166.1\| | | | | |
| | | | | Serine/threonine protein phosphatase PP1, putative, | | | | |
| | | | | expressed [Oryza sativa Japonica Group] | | | | |
| | | | | &gt:gi\|113548140\|dbj\|BAF11583.1\| | | | | |
| | | | | Os03g0268000 [Oryza sativa Japonica Group] | | | | |
| | | | | &gt:gi\|215706450\|dbj\|BAG93306.1\| | | | | |
| | | | | unnamed protein | | | | |
| | | | | product [Oryza sativa Japonica Group] | | | | |
| Predicted | 2333-2353 | NP_001106014 | 162459693 | starch synthase IIb-2 precursor | 1 | Zea mays | 3248 | 4606 |
| siRNA | | | | [Zea mays] | | | | |
| 61212 | | | | &gt:gi\|145202746\|gb\|ABP35814.1\| | | | | |
| | | | | starch synthase IIb-2 precursor | | | | |
| | | | | [Zea mays] | | | | |
| | | ACC86845 | 186695419 | starch synthase IIb precursor | 0.880682 | Sorghum bicolor | 3249 | 4607 |
| | | | | [Sorghum bicolor] | | | | |
| | | ACN31779 | 223975182 | unknown [Zea mays] | 0.87642 | Zea mays | 3250 | 4608 |
| | | NP_001104880 | 162463587 | starch synthase homolog1 [Zea mays] | 0.872159 | Zea mays | 3251 | 4609 |
| | | | | &gt:gi\|2655031\|gb\|AAD13342.1\| | | | | |
| | | | | starch synthase isoform zSTSII-2 | | | | |
| | | | | [Zea mays] | | | | |
| | | Q6Z2T8 | | RecName: Full = Soluble starch synthase 2-2, | 0.764205 | Oryza sativa Japonica Group | 3252 | |
| | | | | chloroplastic/amyloplastic; | | | | |
| | | | | AltName: Full = Soluble starch synthase II-2; | | | | |
| | | | | Flags: Precursor | | | | |
| | | | | &gt:gi\|262345641\|gb\|ACY56184.1\| | | | | |
| | | | | soluble starch synthase II-2 | | | | |
| | | | | [Oryza sativa Japonica Group] | | | | |
| | | | | &gt:gi\|262345643\|gb\|ACY56185.1\| | | | | |
| | | | | soluble starch synthase II-2 | | | | |
| | | | | [Oryza sativa Japonica Group] | | | | |
| | | | | &gt:gi\|262345645\|gb\|ACY56186.1\| | | | | |
| | | | | soluble starch synthase II-2 | | | | |
| | | | | [Oryza sativa Japonica Group] | | | | |
| | | | | &gt:gi\|262345647\|gb\|ACY56187.1\| | | | | |
| | | | | soluble starch synthase II-2 | | | | |
| | | | | [Oryza sativa Japonica Group] | | | | |
| | | | | &gt:gi\|262345649\|gb\|ACY56188.1\| | | | | |
| | | | | soluble starch synthase II-2 | | | | |
| | | | | [Oryza sativa Japonica Group] | | | | |
| | | | | &gt:gi\|262345651\|gb\|ACY56189.1\| | | | | |
| | | | | soluble starch synthase II-2 | | | | |
| | | | | [Oryza sativa Japonica Group] | | | | |
| | | EEC73999 | 54362548 | hypothetical protein OsI_08916 | 0.762784 | Oryza sativa Indica Group | 3253 | |
| | | | | [Oryza sativa Indica Group] | | | | |
| | | | | &gt:gi\|262345653\|gb\|ACY56190.1\| | | | | |
| | | | | soluble starch synthase II-2 | | | | |
| | | | | [Oryza sativa Indica Group] | | | | |
| | | | | &gt:gi\|262345655\|gb\|ACY56191.1\| | | | | |
| | | | | soluble starch synthase II-2 | | | | |
| | | | | [Oryza sativa Indica Group] | | | | |
| | | | | &gt:gi\|262345657\|gb\|ACY56192.1\| | | | | |
| | | | | soluble starch synthase II-2 | | | | |
| | | | | [Oryza sativa Indica Group] | | | | |
| | | | | &gt:gi\|262345659\|gb\|ACY56193.1\| | | | | |
| | | | | soluble starch synthase II-2 | | | | |
| | | | | [Oryza sativa Indica Group] | | | | |
| | | | | &gt:gi\|262345661\|gb\|ACY56194.1\| | | | | |
| | | | | soluble starch synthase II-2 | | | | |
| | | | | [Oryza sativa Indica Group] | | | | |
| | | | | &gt:gi\|262345665\|gb\|ACY56196.1\| | | | | |
| | | | | soluble starch synthase II-2 | | | | |
| | | | | [Oryza sativa Indica Group] | | | | |
| | | | | &gt:gi\|262345667\|gb\|ACY56197.1\| | | | | |
| | | | | soluble starch synthase II-2 | | | | |
| | | | | [Oryza sativa Indica Group] | | | | |
| | | NP_001048104 | 115448648 | Os02g0744700 [Oryza sativa Japonica Group] | 0.75 | Oryza sativa Japonica Group | 3254 | 4610 |
| | | | | &gt:gi\|113537635\|dbj\|BAF10018.1\| | | | | |
| | | | | Os02g0744700 [Oryza sativa Japonica Group] | | | | |
| | | ACY56195 | 262345662 | soluble starch synthase II-2 | 0.761364 | Oryza sativa Indica Group | 3255 | 4611 |
| | | | | [Oryza sativa Indica Group] | | | | |
| | | AAK81729 | 15028466 | soluble starch synthase II-2 | 0.761364 | Oryza sativa | 3256 | 4612 |
| | | | | [Oryza sativa] | | | | |
| | 2530-2550 | XP_002452556 | 242062533 | hypothetical protein SORBIDRAFT_04g028060 | 1 | Sorghum bicolor | 3257 | 4613 |
| | | | | [Sorghum bicolor] | | | | |
| | | | | &gt:gi\|241932387\|gb\|EES05532.1\| | | | | |
| | | | | hypothetical protein SORBIDRAFT_04g028060 | | | | |
| | | | | [Sorghum bicolor] | | | | |
| Predicted | 854-874 | BAJ91900 | 326511510 | predicted protein | 1 | Hordeum vulgare subsp. vulgare | 3258 | 4614 |
| zma | | | | [Hordeum vulgare subsp. vulgare] | | | | |
| mir | | | | | | | | |
| 48327 | | | | | | | | |
| | | BAD52695 | 14164403 | putative protein kinase PK12 | 0.854415 | Oryza sativa Japonica Group | 3259 | 4615 |
| | | | | [Oryza sativa Japonica Group] | | | | |
| | | | | &gt:gi\|215694659\|dbj\|BAG89850.1\| | | | | |
| | | | | unnamed protein product | | | | |
| | | | | [Oryza sativa Japonica Group] | | | | |
| | | | | &gt:gi\|222618771\|gb\|EEE54903.1\| | | | | |
| | | | | hypothetical protein OsJ_02427 | | | | |
| | | | | [Oryza sativa Japonica Group] | | | | |
| | | ACG35326 | 195626991 | serine/threonine-protein kinase | 0.844869 | Zea mays | 3260 | 4616 |
| | | | | AFC3 [Zea mays] | | | | |
| | | ACR34364 | 238006657 | unknown [Zea mays] | 0.844869 | Zea mays | 3261 | 4617 |
| | | ACN28994 | 223949820 | unknown [Zea mays] | 0.837709 | Zea mays | 3262 | 4618 |
| | | NP_001043452 | 115438077 | Os01g0590900 [Oryza sativa Japonica Group] | 0.837709 | Oryza sativa Japonica Group | 3263 | 4619 |
| | | | | &gt:gi\|113532983\|dbj\|BAF05366.1\| | | | | |
| | | | | Os01g0590900 [Oryza sativa Japonica Group] | | | | |
| | | BAJ98839 | 326501215 | predicted protein | 0.768496 | Hordeum vulgare subsp. vulgare | 3264 | 4620 |
| | | | | [Hordeum vulgare subsp. vulgare] | | | | |
| | | XP_002455867 | 242053442 | hypothetical protein SORBIDRAFT_03g026540 | 0.708831 | Sorghum bicolor | 3265 | 4621 |
| | | | | [Sorghum bicolor] | | | | |
| | | | | &gt:gi\|241927842\|gb\|EES00987.1\| | | | | |
| | | | | hypothetical protein SORBIDRAFT_03g026540 | | | | |
| | | | | [Sorghum bicolor] | | | | |
| | | XP_002308625 | 224089073 | predicted protein [Populus trichocarpa] | 0.71599 | Populus trichocarpa | 3266 | 4622 |
| | | | | &gt:gi\|222854601\|gb\|EEE92148.1\| | | | | |
| | | | | predicted protein [Populus trichocarpa] | | | | |
| | 368-388 | XP_002456429 | 242054566 | hypothetical protein SORBIDRAFT_03g036130 | 1 | Sorghum bicolor | 3267 | 4623 |
| | | | | [Sorghum bicolor] | | | | |
| | | | | &gt:gi\|241928404\|gb\|EES01549.1\| | | | | |
| | | | | hypothetical protein SORBIDRAFT_03g036130 | | | | |
| | | | | [Sorghum bicolor] | | | | |
| | | NP_001146289 | 226499967 | hypothetical protein LOC100279864 [Zea mays] | 0.812362 | Zea mays | 3268 | 4624 |
| | | | | &gt:gi\|219886519\|gb\|ACL53634.1\| | | | | |
| | | | | unknown [Zea mays] | | | | |
| | 946-966 | EEC70991 | 54362548 | hypothetical protein OsI_02649 | 1 | Oryza sativa Indica Group | 3269 | |
| | | | | [Oryza sativa Indica Group] | | | | |
| Predicted | 179-200 | BAK06079 | 326499176 | predicted protein | 1 | Hordeum vulgare subsp. vulgare | 3270 | 4625 |
| zma | | | | [Hordeum vulgare subsp. vulgare] | | | | |
| mir | | | | | | | | |
| 48479 | | | | | | | | |
| | | NP_001140935 | 226492610 | hypothetical protein LOC100273013 [Zea mays] | 0.860399 | Zea mays | 3271 | 4626 |
| | | | | &gt:gi\|194701826\|gb\|ACF84997.1\| | | | | |
| | | | | unknown [Zea mays] | | | | |
| | | | | &gt:gi\|194703532\|gb\|ACF85850.1\| | | | | |
| | | | | unknown [Zea mays] | | | | |
| | | NP_001051729 | 115456256 | Os03g0821800 [Oryza sativa Japonica Group] | 0.874644 | Oryza sativa Japonica Group | 3272 | 4627 |
| | | | | &gt:gi\|108711803\|gb\|ABF99598.1\| | | | | |
| | | | | glycosyl transferase, group 2 family protein, expressed | | | | |
| | | | | [Oryza sativa Japonica Group] | | | | |
| | | | | &gt:gi\|113550200\|dbj\|BAF13643.1\| | | | | |
| | | | | Os03g0821800 [Oryza sativa Japonica Group] | | | | |
| | | | | &gt:gi\|215717119\|dbj\|BAG95482.1\| unnamed | | | | |
| | | | | protein product [Oryza sativa Japonica Group] | | | | |
| | | | | &gt:gi\|222626064\|gb\|EEE60196.1\| | | | | |
| | | | | hypothetical protein OsJ_13154 | | | | |
| | | | | [Oryza sativa Japonica Group] | | | | |
| | | AAO18453 | 24899477 | putative dolichyl-phosphate beta-glucosyltransferase | 0.794872 | Oryza sativa Japonica Group | 3273 | 4628 |
| | | | | [Oryza sativa Japonica Group] | | | | |
| | 467-488 | ACN31661 | 223974946 | unknown [Zea mays] | 1 | Zea mays | 3274 | 4629 |
| | | NP_001142978 | 226498261 | hypothetical protein LOC100275432 [Zea mays] | 0.972387 | Zea mays | 3275 | 4630 |
| | | | | &gt:gi\|195612426\|gb\|ACG28043.1\| | | | | |
| | | | | hypothetical protein [Zea mays] | | | | |
| | | XP_002458459 | 242058626 | hypothetical protein SORBIDRAFT_03g034030 | 0.767258 | Sorghum bicolor | 3276 | 4631 |
| | | | | [Sorghum bicolor] | | | | |
| | | | | &gt:gi\|241930434\|gb\|EES03579.1\| | | | | |
| | | | | hypothetical protein SORBIDRAFT_03g034030 | | | | |
| | | | | [Sorghum bicolor] | | | | |
| | 114-135 | ACG40719 | 195642501 | alpha-taxilin [Zea mays] | 1 | Zea mays | 3277 | 4632 |
| | | NP_001132592 | 212722627 | hypothetical protein LOC100194064 [Zea mays] | 0.99763 | Zea mays | 3278 | 4633 |
| | | | | &gt:gi\|194694846\|gb\|ACF81507.1\| | | | | |
| | | | | unknown [Zea mays] | | | | |
| | | XP_002468045 | 242041300 | hypothetical protein SORBIDRAFT_01g038590 | 0.890995 | Sorghum bicolor | 3279 | 4634 |
| | | | | [Sorghum bicolor] | | | | |
| | | | | &gt:gi\|241921899\|gb\|EER95043.1\| | | | | |
| | | | | hypothetical protein SORBIDRAFT_01g038590 | | | | |
| | | | | [Sorghum bicolor] | | | | |
| | | NP_001141235 | 226500461 | hypothetical protein LOC100273322 | 0.869668 | Zea mays | 3280 | 4635 |
| | | | | [Zea mays] | | | | |
| | | | | &gt:gi\|194703420\|gb\|ACF85794.1\| | | | | |
| | | | | unknown [Zea mays] | | | | |
| | | ACN28388 | 223948608 | unknown [Zea mays] | 0.872038 | Zea mays | 3281 | 4636 |
| | | ACL54606 | 219888462 | unknown [Zea mays] | 0.869668 | Zea mays | 3282 | 4637 |
| | | ACG42375 | 195645813 | alpha-taxilin [Zea mays] | 0.834123 | Zea mays | 3283 | 4638 |
| | 192-213 | EEC76428 | 54362548 | hypothetical protein OsI_14108 | 1 | Oryza sativa Indica Group | 3284 | |
| | | | | [Oryza sativa Indica Group] | | | | |
| Predicted | 254-275 | XP_002458718 | 242059144 | hypothetical protein SORBIDRAFT_03g038910 | 1 | Sorghum bicolor | 3285 | 4639 |
| zma | | | | [Sorghum bicolor] | | | | |
| mir | | | | &gt:gi\|241930693\|gb\|EES03838.1\| | | | | |
| 48482 | | | | hypothetical protein SORBIDRAFT_03g038910 | | | | |
| | | | | [Sorghum bicolor] | | | | |
| | | NP_001148462 | 226497951 | LOC100282077 [Zea mays] | 0.936306 | Zea mays | 3286 | 4640 |
| | | | | &gt:gi\|195619496\|gb\|ACG31578.1\| | | | | |
| | | | | CTD small phosphatase-like protein | | | | |
| | | | | [Zea mays] | | | | |
| | | NP_001136647 | 219363036 | hypothetical protein LOC100216776 | 0.933121 | Zea mays | 3287 | 4641 |
| | | | | [Zea mays] | | | | |
| | | | | &gt:gi\|194696496\|gb\|ACF82332.1\| | | | | |
| | | | | unknown [Zea mays] | | | | |
| | | ACG32959 | 195622257 | CTD small phosphatase-like protein | 0.933121 | Zea mays | 3288 | 4642 |
| | | | | [Zea mays] | | | | |
| | | EEC71746 | 54362548 | hypothetical protein OsI_04315 | 0.898089 | Oryza sativa Indica Group | 3289 | |
| | | | | [Oryza sativa Indica Group] | | | | |
| | | NP_001044710 | 115440860 | Os01g0833100 [Oryza sativa Japonica Group] | 0.894904 | Oryza sativa Japonica Group | 3290 | 4643 |
| | | | | &gt:gi\|56202314\|dbj\|BAD73773.1\| | | | | |
| | | | | NLI interacting factor (NIF) family protein-like | | | | |
| | | | | [Oryza sativa Japonica Group] | | | | |
| | | | | &gt:gi\|113534241\|dbj\|BAF06624.1\| | | | | |
| | | | | Os01g0833100 [Oryza sativa Japonica Group] | | | | |
| | | | | &gt:gi\|215697182\|dbj\|BAG91176.1\| | | | | |
| | | | | unnamed protein product | | | | |
| | | | | [Oryza sativa Japonica Group] | | | | |
| | | | | &gt:gi\|222619498\|gb\|EEE55630.1\| | | | | |
| | | | | hypothetical protein OsJ_03971 | | | | |
| | | | | [Oryza sativa Japonica Group] | | | | |
| | | BAJ91589 | 326509344 | predicted protein | 0.89172 | Hordeum vulgare subsp. vulgare | 3291 | 4644 |
| | | | | [Hordeum vulgare subsp. vulgare] | | | | |
| | | XP_002439933 | 242088200 | hypothetical protein SORBIDRAFT_09g022940 | 0.719745 | Sorghum bicolor | 3292 | 4645 |
| | | | | [Sorghum bicolor] | | | | |
| | | | | &gt:gi\|241945218\|gb\|EES18363.1\| | | | | |
| | | | | hypothetical protein SORBIDRAFT_09g022940 | | | | |
| | | | | [Sorghum bicolor] | | | | |
| | | EAY98360 | 54362548 | hypothetical protein OsI_20271 | 0.748408 | Oryza sativa Indica Group | 3293 | |
| | | | | [Oryza sativa Indica Group] | | | | |
| | | BAK00193 | 326519639 | predicted protein | 0.726115 | Hordeum vulgare subsp. vulgare | 3294 | 4646 |
| | | | | [Hordeum vulgare subsp. vulgare] | | | | |
| | | | | &gt:gi\|326529107\|dbj\|BAK00947.1\| | | | | |
| | | | | predicted protein | | | | |
| | | | | [Hordeum vulgare subsp. vulgare] | | | | |
| | 132-153 | XP_002463784 | 242032778 | hypothetical protein SORBIDRAFT_01g006100 | 1 | Sorghum bicolor | 3295 | 4647 |
| | | | | [Sorghum bicolor] | | | | |
| | | | | &gt:gi\|241917638\|gb\|EER90782.1\| | | | | |
| | | | | hypothetical protein SORBIDRAFT_01g006100 | | | | |
| | | | | [Sorghum bicolor] | | | | |
| | | ACG39703 | 195640469 | ferredoxin--NADP reductase, root isozyme | 0.936842 | Zea mays | 3296 | 4648 |
| | | | | [Zea mays] | | | | |
| | | NP_001051476 | 115455750 | Os03g0784700 [Oryza sativa Japonica Group] | 0.894737 | Oryza sativa Japonica Group | 3297 | 4649 |
| | | | | &gt:gi\|729480\|sp\|P41345.1\|FENR2_ORYSJ | | | | |
| | | | | RecName: Full = Ferredoxin--NADP reductase, root isozyme, | | | | |
| | | | | chloroplastic; Short = FNR; Flags: Precursor | | | | |
| | | | | &gt:gi\|14718314\|gb\|AAK72892.1\|AC091123_11 | | | | |
| | | | | ferredoxin-NADP+ reductase | | | | |
| | | | | [Oryza sativa Japonica Group] | | | | |
| | | | | &gt:gi\|435647\|dbj\|BAA04232.1\| | | | | |
| | | | | ferredoxin-NADP+ reductase | | | | |
| | | | | [Oryza sativa Japonica Group] | | | | |
| | | | | &gt:gi\|902936\|dbj\|BAA07479.1\| | | | | |
| | | | | root ferredoxin-NADP+ reductase | | | | |
| | | | | [Oryza sativa Japonica Group] | | | | |
| | | | | &gt:gi\|108711425\|gb\|ABF99220.1 | | | | |
| | | | | Ferredoxin-NADP reductase, root isozyme, | | | | |
| | | | | chloroplast precursor, putative, expressed | | | | |
| | | | | [Oryza sativa Japonica Group] | | | | |
| | | | | &gt:gi\|113549947\|dbj\|BAF13390.1\| | | | | |
| | | | | Os03g0784700 [Oryza sativa Japonica Group] | | | | |
| | | | | &gt:gi\|125545944\|gb\|EAY92083.1\| | | | | |
| | | | | hypothetical protein OsI_13788 | | | | |
| | | | | [Oryza sativa Indica Group] | | | | |
| | | | | &gt:gi\|125588154\|gb\|EAZ28818.1\| | | | | |
| | | | | hypothetical protein OsJ_12850 | | | | |
| | | | | [Oryza sativa Japonica Group] | | | | |
| | | | | &gt:gi\|215678914\|dbj\|BAG96344.1\| | | | | |
| | | | | unnamed protein product | | | | |
| | | | | [Oryza sativa Japonica Group] | | | | |
| | | | | &gt:gi\|1096932lprf\|\|2113196A ferredoxin-NADP | | | | |
| | | | | oxidoreductase | | | | |
| | | BAJ93902 | 326488466 | predicted protein | 0.878947 | Hordeum vulgare subsp. vulgare | 3298 | 4650 |
| | | | | [Hordeum vulgare subsp. vulgare] | | | | |
| | | NP_001058890 | 115470582 | Os07g0147900 [Oryza sativa Japonica Group] | 0.823684 | Oryza sativa Japonica Group | 3299 | 4651 |
| | | | | &gt:gi\|3913653\|sp\|O23877.1\|FENR3_ORYSJ | | | | |
| | | | | RecName: Full = Ferredoxin--NADP reductase, embryo | | | | |
| | | | | isozyme, chloroplastic; Short = FNR; Flags: Precursor | | | | |
| | | | | &gt:gi\|1778686\|dbj\|BAA13417.1\| precursor | | | | |
| | | | | ferredoxin-NADP+ oxidoreductase [Oryza sativa | | | | |
| | | | | (Japonica cultivar-group)] | | | | |
| | | | | &gt:gi\|34393644\|dbj\|BAC83340.1\| Ferredoxin-NADP | | | | |
| | | | | reductase, embryo isozyme, chloroplasl precursor | | | | |
| | | | | (FNR) [Oryza sativa Japonica Group] | | | | |
| | | | | &gt:gi\|11361 0426\|dbj\|BAF20804.1\| Os07g0147900 | | | | |
| | | | | [Oryza sativa Japonica Group] | | | | |
| | | | | &gt:gi\|125599119\|gb\|EAZ38695.1\| hypothetical | | | | |
| | | | | protein OsJ_23093 [Oryza sativa Japonica Group] | | | | |
| | | NP_001132762 | 212722161 | hypothetical protein LOC100194249 [Zea mays] | 0.823684 | Zea mays | 3300 | 4652 |
| | | | | &gt:gi\|194695328\|gb\|ACF81748.1\| | | | | |
| | | | | unknown [Zea mays] | | | | |
| | | ACG35047 | 195626433 | ferredoxin-NADP reductase, embryo isozyme [Zea mays] | 0.821053 | Zea mays | 3301 | 4653 |
| | | EAZ02773 | 54362548 | hypothetical protein OsI_24896 | 0.821053 | Oryza sativa Indica Group | 3302 | |
| | | | | [Oryza sativa Indica Group] | | | | |
| | | XP_002461452 | 242047411 | hypothetical protein SORBIDRAFT_02g002900 | 0.815789 | Sorghum bicolor | 3303 | 4654 |
| | | | | [Sorghum bicolor] | | | | |
| | | | | &gt:gi\|241924829\|gb\|EER97973.1\| | | | | |
| | | | | hypothetical protein SORBIDRAFT_02g002900 | | | | |
| | | | | [Sorghum bicolor] | | | | |
| | 172-193 | ACF87736 | 194707303 | unknown [Zea mays] | 1 | Zea mays | 3304 | 4655 |
| | | | | &gt:gi\|223949881\|gb\|ACN29024.1\| | | | | |
| | | | | unknown [Zea mays] | | | | |
| | | | | &gt:gi\|238009776\|gb\|ACR35923.1\| | | | | |
| | | | | unknown [Zea mays] | | | | |
| | | | | &gt:gi\|238011352\|gb\|ACR36711.1\| | | | | |
| | | | | unknown [Zea mays] | | | | |
| | | AAB40034 | 500750 | ferredoxin-NADP reductase precursor | 0.847769 | Zea mays | 3305 | 4656 |
| | | | | [Zea mays] | | | | |
| | | 1JB9_A | | Chain A, Crystal Structure Of The Ferredoxin: nadp+ | 0.829396 | Zea mays | 3306 | |
| | | | | Reductase From Maize Root At 1.7 Angstroms | | | | |
| | 302-323 | XP_002465048 | 242035306 | hypothetical protein SORBIDRAFT_01g031270 | 1 | Sorghum bicolor | 3307 | 4657 |
| | | | | [Sorghum bicolor] | | | | |
| | | | | &gt:gi\|241918902\|gb\|EER92046.1\| | | | | |
| | | | | hypothetical protein SORBIDRAFT_01g031270 | | | | |
| | | | | [Sorghum bicolor] | | | | |
| | | NP_001151845 | 226530638 | actin-depolymerizing factor [Zea mays] | 0.954248 | Zea mays | 3308 | 4658 |
| | | | | &gt:gi\|195650207\|gb\|ACG44571.1\| | | | | |
| | | | | actin-depolymerizing factor [Zea mays] | | | | |
| | | AAG13444 | 10122030 | putative actin depolymerizing factor | 0.954248 | Oryza sativa Japonica Group | 3309 | 4659 |
| | | | | [Oryza sativa Japonica Group] | | | | |
| | | | | &gt:gi\|22122913\|gb\|AAM92296.1\| | | | | |
| | | | | putative actin depolymerizing factor | | | | |
| | | | | [Oryza sativa Japonica Group] | | | | |
| | | | | &gt:gi\|125532673\|gb\|EAY79238.1\| | | | | |
| | | | | hypothetical protein OsI_34355 | | | | |
| | | | | [Oryza sativa Indica Group] | | | | |
| | | NP_001065088 | 115482989 | Os10g0521100 [Oryza sativa Japonica Group] | 0.895425 | Oryza sativa Japonica Group | 3310 | 4660 |
| | | | | &gt:gi\|122212110\|sp\|Q337A5.1\|ADF10_ORYSJ | | | | |
| | | | | RecName: Full = Actin-depolymerizing factor 10; | | | | |
| | | | | Short = ADF-10; Short = OsADF10 | | | | |
| | | | | &gt:gi\|78708922\|gb\|ABB47897.1\| | | | | |
| | | | | Actin-depolymerizing factor, putative, expressed | | | | |
| | | | | [Oryza sativa Japonica Group] | | | | |
| | | | | &gt:gi\|113639697\|dbj\|BAF27002.1\| | | | | |
| | | | | Os10g0521100 [Oryza sativa Japonica Group] | | | | |
| | | | | &gt:gi\|215693794\|dbj\|BAG88993.1\| | | | | |
| | | | | unnamed protein product | | | | |
| | | | | [Oryza sativa Japonica Group] | | | | |
| | | | | &gt:gi\|215768406\|dbj\|BAH00635.1\| | | | | |
| | | | | unnamed protein product | | | | |
| | | | | [Oryza sativa Japonica Group] | | | | |
| | | | | &gt:gi\|222613 147\|gb\|EEE51279.1\| | | | | |
| | | | | hypothetical protein OsJ_32187 | | | | |
| | | | | [Oryza sativa Japonica Group] | | | | |
| | | Q0D744 | | RecName: Full = Putative actin-depolymerizing factor 8; | 0.843137 | Oryza sativa Japonica Group | 3311 | |
| | | | | Short = ADF-8; Short = OsADF8 | | | | |
| | | | | &gt:gi\|34394310\|dbj\|BAC84792.1\| | | | | |
| | | | | putative actin depolymerizing factor | | | | |
| | | | | [Oryza sativa Japonica Group] | | | | |
| | | EEE58130 | 54398660 | hypothetical protein OsJ_09029 | 0.816993 | Oryza sativa Japonica Group | 3312 | |
| | | | | [Oryza sativa Japonica Group] | | | | |
| Predicted | 893-913 | XP_002465226 | 242035662 | hypothetical protein SORBIDRAFT_01g034550 | 1 | Sorghum bicolor | 3313 | 4661 |
| zma | | | | [Sorghum bicolor] | | | | |
| mir | | | | &gt:gi\|241919080\|gb\|EER92224.1\| | | | | |
| 49248 | | | | hypothetical protein SORBIDRAFT_01g034550 | | | | |
| | | | | [Sorghum bicolor] | | | | |
| | | ABF96114 | 108705663 | proline-rich family protein, putative, | 0.8583 | Oryza sativa Japonica Group | 3314 | 4662 |
| | | | | expressed [Oryza sativa Japonica Group] | | | | |
| | | | | &gt:gi\|108708320\|gb\|ABF96115.1\| | | | | |
| | | | | proline-rich family protein, putative, expressed | | | | |
| | | | | [Oryza sativa Japonica Group] | | | | |
| | | | | &gt:gi\|218192888\|gb\|EEC75315.1\| | | | | |
| | | | | hypothetical protein OsI_11686 | | | | |
| | | | | [Oryza sativa Indica Group] | | | | |
| | | | | &gt:gi\|222624967\|gb\|EEE59099.1\| | | | | |
| | | | | hypothetical protein OsJ_10953 | | | | |
| | | | | [Oryza sativa Japonica Group] | | | | |
| | | NP_001060438 | 115473678 | Os07g0642800 [Oryza sativa Japonica Group] | 0.722672 | Oryza sativa Japonica Group | 3315 | 4663 |
| | | | | &gt:gi\|33146645\|dbj\|BAC79975.1\| unknown protein | | | | |
| | | | | [Oryza sativa Japonica Group] | | | | |
| | | | | &gt:gi\|50509935\|dbj\|BAD30256.1\| unknown protein | | | | |
| | | | | [Oryza sativa Japonica Group] | | | | |
| | | | | &gt:gi\|113611 974\|dbj\|BAF22352.1\| Os07g0642800 | | | | |
| | | | | [Oryza sativa Japonica Group] | | | | |
| | | | | &gt:gi\|125601263\|gb\|EAZ40839.1\| hypothetical | | | | |
| | | | | protein OsJ_25318 [Oryza sativa Japonica Group] | | | | |
| | | | | &gt:gi\|215707132\|dbj\|BAG93592.1\| unnamed protein | | | | |
| | | | | product [Oryza sativa Japonica Group] | | | | |
| | | NP_001136832 | 219362356 | hypothetical protein LOC100216981 | 0.714575 | Zea mays | 3316 | 4664 |
| | | | | [Zea mays] | | | | |
| | | | | &gt:gi\|194697292\|gb\|ACF82730.1\| | | | | |
| | | | | unknown [Zea mays] | | | | |
| | | | | &gt:gi\|223944185\|gb\|ACN26176.1\| | | | | |
| | | | | unknown [Zea mays] | | | | |
| | | EAZ04885 | 54362548 | hypothetical protein OsI_27067 | 0.720648 | Oryza sativa Indica Group | 3317 | |
| | | | | [Oryza sativa Indica Group] | | | | |
| | | XP_002461113 | 242046733 | hypothetical protein SORBIDRAFT_02g040980 | 0.720648 | Sorghum bicolor | 3318 | 4665 |
| | | | | [Sorghum bicolor] | | | | |
| | | | | &gt:gi\|241924490\|gb\|EER97634.1\| | | | | |
| | | | | hypothetical protein SORBIDRAFT_02g040980 | | | | |
| | | | | [Sorghum bicolor] | | | | |
| | | BAK07098 | 326515703 | predicted protein | 0.718623 | Hordeum vulgare subsp. vulgare | 3319 | 4666 |
| | | | | [Hordeum vulgare subsp. vulgare] | | | | |
| | 320-340 | YP_003208225 | 260677373 | ribosomal protein S3 [Coix lacryma-jobi] | 1 | Coix lacryma-jobi | 3320 | 4667 |
| | | | | &gt:gi\|209361395\|gb\|ACI43310.1\| ribosomal protein | | | | |
| | | | | S3 [Coix lacryma-jobi] | | | | |
| | | YP_004733282 | 339906432 | ribosomal protein S3 [Indocalamus longiauritus] | 0.959821 | Indocalamus longiauritus | 3321 | 4668 |
| | | | | &gt:gi\|340034064\|ref\|YP_004733616.1\| ribosomal | | | | |
| | | | | protein S3 [Phyllostachys edulis] | | | | |
| | | | | &gt:gi\|340034235\|ref\|YP_004733798.1\| ribosomal | | | | |
| | | | | protein S3 [Acidosasa purpurea] | | | | |
| | | | | &gt:gi\|340034403\|ref\|YP_004734016.1\| ribosomal protein | | | | |
| | | | | S3 [Phyllostachys nigra var. henonis] | | | | |
| | | | | &gt:gi\|340034574\|ref\|YP_004734223.1\| ribosomal | | | | |
| | | | | protein S3 [Ferrocalamus rimosivaginus] | | | | |
| | | | | &gt:gi\|307133924\|gb\|ADN32929.1\| ribosomal protein | | | | |
| | | | | S3 [Phyllostachys nigra var. henonis] | | | | |
| | | | | &gt:gi\|309321655\|gb\|ADO65180.1\| ribosomal protein | | | | |
| | | | | S3 [Acidosasa purpurea] | | | | |
| | | | | &gt:gi\|309321739\|gb\|ADO65263.1\| ribosomal protein | | | | |
| | | | | S3 [Ferrocalamus rimosivaginus] | | | | |
| | | | | &gt:gi\|309321823\|gb\|ADO65346.1\| ribosomal protein | | | | |
| | | | | S3 [Indocalamus longiauritus] | | | | |
| | | | | &gt:gi\|309321906\|gb\|ADO65428.1\| ribosomal protein | | | | |
| | | | | S3 [Phyllostachys edulis] | | | | |
| | | AEI70820 | 337730951 | ribosomal protein S3 [Puelia olyriformis] | 0.955357 | Puelia olyriformis | 3322 | 4669 |
| | | YP_004734139 | 340034430 | ribosomal protein S3 [Bambusa emeiensis] | 0.946429 | Bambusa emeiensis | 3323 | 4670 |
| | | | | &gt:gi\|309321991\|gb\|ADO65512.1\| | | | | |
| | | | | ribosomal protein S3 | | | | |
| | | | | [Bambusa emeiensis] | | | | |
| Predicted | 482-503 | NP_001140256 | 226500157 | hypothetical protein LOC100272298 [Zea mays] | 1 | Zea mays | 3324 | 4671 |
| zma | | | | &gt:gi\|223947639\|gb\|ACN27903.1\| | | | | |
| mir | | | | unknown [Zea mays] | | | | |
| 49259 | | | | | | | | |
| | 374-395 | BAJ84854 | 326490380 | predicted protein | 1 | Hordeum vulgare subsp. vulgare | 3325 | 4672 |
| | | | | [Hordeum vulgare subsp. vulgare] | | | | |
| | | XP_002446506 | 242073139 | hypothetical protein SORBIDRAFT_06g017 100 | 0.901328 | Sorghum bicolor | 3326 | 4673 |
| | | | | [Sorghum bicolor] | | | | |
| | | | | &gt:gi\|241937689\|gb\|EES10834.1\| | | | | |
| | | | | hypothetical protein SORBIDRAFT_06g017100 | | | | |
| | | | | [Sorghum bicolor] | | | | |
| | | NP_001148156 | 226498467 | amino acid permease [Zea mays] | 0.893738 | Zea mays | 3327 | 4674 |
| | | | | &gt:gi\|195616234\|gb\|ACG29947.1\| | | | | |
| | | | | amino acid permease [Zea mays] | | | | |
| | | NP_001052846 | 115458491 | Os04g0435100 [Oryza sativa Japonica Group] | 0.86907 | Oryza sativa Japonica Group | 3328 | 4675 |
| | | | | &gt:gi\|21741208\|emb\|CAD41019.1\| | | | | |
| | | | | OSJNBb0086G13.12 [Oryza sativa Japonica Group] | | | | |
| | | | | &gt:gi\|113564417\|dbj\|BAF14760.1\| | | | | |
| | | | | Os04g0435100 [Oryza sativa Japonica Group] | | | | |
| | | | | &gt:gi\|116310284\|emb\|CAH67303.1\| | | | | |
| | | | | OSIGBa0102D10.6 [Oryza sativa Indica Group] | | | | |
| | | | | &gt:gi\|125548375\|gb\|EAY94197.1\| | | | | |
| | | | | hypothetical protein OsI_15971 [Oryza sativa Indica Group] | | | | |
| | | | | &gt:gi\|125590463\|gb\|EAZ30813.1\| | | | | |
| | | | | hypothetical protein OsJ_14882 | | | | |
| | | | | [Oryza sativa Japonica Group] | | | | |
| | | | | &gt:gi\|215687171\|dbj\|BAG90941.1\| | | | | |
| | | | | unnamed protein product | | | | |
| | | | | [Oryza sativa Japonica Group] | | | | |
| | | XP_002309938 | 224093574 | amino acid transporter | 0.827324 | Populus trichocarpa | 3329 | 4676 |
| | | | | [Populus trichocarpa] | | | | |
| | | | | &gt:gi\|222852841\|gb\|EEE90388.1\| | | | | |
| | | | | amino acid transporter | | | | |
| | | | | [Populus trichocarpa] | | | | |
| | | CBI21593 | 270232045 | unnamed protein product [Vitis vinifera] | 0.823529 | Vitis vinifera | 3330 | 4677 |
| | | XP_002274762 | 225438399 | PREDICTED: hypothetical protein | 0.821632 | Vitis vinifera | 3331 | 4678 |
| | | | | [Vitis vinifera] | | | | |
| | | CAN60789 | 147773951 | hypothetical protein VITISV_000645 | 0.819734 | Vitis vinifera | 3332 | 4679 |
| | | | | [Vitis vinifera] | | | | |
| | | XP_002520314 | 255558577 | GABA-specific permease, putative | 0.741935 | Ricinus communis | 3333 | 4680 |
| | | | | [Ricinus communis] | | | | |
| | | | | &gt:gi\|223540533\|gb\|EEF42100.1\| | | | | |
| | | | | GABA-specific permease, putative | | | | |
| | | | | [Ricinus communis] | | | | |
| | | XP_002284603 | 225459654 | PREDICTED: hypothetical protein | 0.753321 | Vitis vinifera | 3334 | 4681 |
| | | | | [Vitis vinifera] | | | | |
| | 28-49 | XP_002456818 | 242055344 | hypothetical protein SORBIDRAFT_03g043360 | 1 | Sorghum bicolor | 3335 | 4682 |
| | | | | [Sorghum bicolor] | | | | |
| | | | | &gt:gi\|241928793\|gb\|EES01938.1\| | | | | |
| | | | | hypothetical protein SORBIDRAFT_03g043360 | | | | |
| | | | | [Sorghum bicolor] | | | | |
| | | NP_001144608 | 226505097 | hypothetical protein LOC100277624 [Zea mays] | 0.899244 | Zea mays | 3336 | 4683 |
| | | | | &gt:gi\|195644550\|gb\|ACG41743.1\| | | | | |
| | | | | hypothetical protein [Zea mays] | | | | |
| | | | | &gt:gi\|224033445\|gb\|ACN35798.1\| | | | | |
| | | | | unknown [Zea mays] | | | | |
| | | NP_001062225 | 115477257 | Os08g0513300 [Oryza sativa Japonica Group] | 0.743073 | Oryza sativa Japonica Group | 3337 | 4684 |
| | | | | &gt:gi\|42408809\|dbj\|BAD10070.1\| | | | | |
| | | | | unknown protein [Oryza sativa Japonica Group] | | | | |
| | | | | &gt:gi\|113624194\|dbj\|BAF24139.1\| | | | | |
| | | | | Os08g0513300 [Oryza sativa Japonica Group] | | | | |
| | | | | &gt:gi\|125562163\|gb\|EAZ07611.1\| hypothetical | | | | |
| | | | | protein OsI_29862 [Oryza sativa Indica Group] | | | | |
| | | | | &gt:gi\|125603995\|gb\|EAZ43320.1\| hypothetical | | | | |
| | | | | protein OsJ_27916 | | | | |
| | | | | [Oryza sativa Japonica Group] | | | | |
| | | | | &gt:gi\|215697131\|dbj\|BAG91125.1\| | | | | |
| | | | | unnamed protein product | | | | |
| | | | | [Oryza sativa Japonica Group] | | | | |
| | | BAJ93470 | 326528576 | predicted protein | 0.70529 | Hordeum vulgare subsp. vulgare | 3338 | 4685 |
| | | | | [Hordeum vulgare subsp. vulgare] | | | | |
| | 950-971 | XP_002461022 | 242046301 | hypothetical protein SORBIDRAFT_02g039350 | 1 | Sorghum bicolor | 3339 | 4686 |
| | | | | [Sorghum bicolor] | | | | |
| | | | | &gt:gi\|241924399\|gb\|EER97543.1\| | | | | |
| | | | | hypothetical protein SORBIDRAFT_02g039350 | | | | |
| | | | | [Sorghum bicolor] | | | | |
| | 266-287 | NP_001147593 | 226502838 | receptor-like protein kinase [Zea mays] | 1 | Zea mays | 3340 | 4687 |
| | | | | &gt:gi\|195612392\|gb\|ACG28026.1\| | | | | |
| | | | | receptor-like protein kinase [Zea mays] | | | | |
| | | NP_001064013 | 115480839 | Os10g0101000 [Oryza sativa Japonica Group] | 0.736196 | Oryza sativa Japonica Group | 3341 | 4688 |
| | | | | &gt:gi\|18481964\|gb\|AAL73562.1\|AC079632_6 | | | | |
| | | | | Putative receptor-like protein kinase | | | | |
| | | | | [Oryza sativa Japonica Group] | | | | |
| | | | | &gt:gi\|19920204\|gb\|AAM08636.1\|AC108883_9 | | | | |
| | | | | Putative receptor-like protein kinase | | | | |
| | | | | [Oryza sativa Japonica Group] | | | | |
| | | | | &gt:gi\|31429736\|gb\|AAP51745.1\| | | | | |
| | | | | Protein kinase domain containing protein, expressed | | | | |
| | | | | [Oryza sativa Japonica Group] | | | | |
| | | | | &gt:gi\|113638622\|dbj\|BAF25927.1\| | | | | |
| | | | | Os10g0101000 [Oryza sativa Japonica Group] | | | | |
| | | | | &gt:gi\|125573756\|gb\|EAZ15040.1\| | | | | |
| | | | | hypothetical protein OsJ_30450 | | | | |
| | | | | [Oryza sativa Japonica Group] | | | | |
| | | BAJ93355 | 326528346 | predicted protein | 0.71411 | Hordeum vulgare subsp. vulgare | 3342 | 4689 |
| | | | | [Hordeum vulgare subsp. vulgare] | | | | |
| | | BAJ85838 | 326495483 | predicted protein | 0.712883 | Hordeum vulgare subsp. vulgare | 3343 | 4690 |
| | | | | [Hordeum vulgare subsp. vulgare] | | | | |
| Predicted | 732-753 | NP_001132844 | 239046576 | hypothetical protein LOC100194336 [Zea mays] | 1 | Zea mays | 3344 | 4691 |
| zma | | | | &gt:gi\|194695554\|gb\|ACF81861.1\| | | | | |
| mir | | | | unknown [Zea mays] | | | | |
| 49642 | | | | &gt:gi\|219885465\|gb\|ACL53107.1\| | | | | |
| | | | | unknown [Zea mays] | | | | |
| | | NP_001140555 | 226493194 | hypothetical protein LOC100272620 [Zea mays] | 0.85348 | Zea mays | 3345 | 4692 |
| | | | | &gt:gi\|194690356\|gb\|ACF79262.1\| | | | | |
| | | | | unknown [Zea mays] | | | | |
| | | | | &gt:gi\|194699966\|gb\|ACF84067.1\| unknown | | | | |
| | | | | [Zea mays] | | | | |
| | | | | &gt:gi\|219887213\|gb\|ACL53981.1\| | | | | |
| | | | | unknown [Zea mays] | | | | |
| | | XP_002460380 | 242045017 | hypothetical protein SORBIDRAFT_02g027300 | 0.827839 | Sorghum bicolor | 3346 | 4693 |
| | | | | [Sorghum bicolor] | | | | |
| | | | | &gt:gi\|241923757\|gb\|EER96901.1\| | | | | |
| | | | | hypothetical protein SORBIDRAFT_02g027300 | | | | |
| | | | | [Sorghum bicolor] | | | | |
| | | EEE69885 | 54398660 | hypothetical protein OsJ_29706 | 0.750916 | Oryza sativa Japonica Group | 3347 | |
| | | | | [Oryza sativa Japonica Group] | | | | |
| | | NP_001063436 | 115479684 | Os09g0470500 [Oryza sativa Japonica Group] | 0.754579 | Oryza sativa Japonica Group | 3348 | 4694 |
| | | | | &gt:gi\|75125073\|sp\|Q6K498.1\|HOX4_ORYSJ | | | | |
| | | | | RecName: Full = Homeobox-leucine zipper protein HOX4; | | | | |
| | | | | AltName: Full = HD-ZIP protein HOX4; | | | | |
| | | | | AltName: Full = Homeodomain transcription factor HOX4; | | | | |
| | | | | AltName: Full = OsHox4 | | | | |
| | | | | &gt:gi\|75315199\|sp\|Q9XH37.1\|HOX4_ORYSI | | | | |
| | | | | RecName: Full = Homeobox-leucine zipper protein HOX4; | | | | |
| | | | | AltName: Full = HD-ZIP protein HOX4; | | | | |
| | | | | AltName: Full = Homeodomain transcription factor HOX4; | | | | |
| | | | | AltName: Full = OsHox4 | | | | |
| | | | | &gt:gi\|5006853\|gb\|AAD37697.1\|AF145728_1 | | | | |
| | | | | homeodomain leucine zipper protein | | | | |
| | | | | [Oryza sativa Indica Group] | | | | |
| | | | | &gt:gi\|47848413\|dbj\|BAD22271.1\| | | | | |
| | | | | homeodomain leucine zipper protein | | | | |
| | | | | [Oryza sativa Japonica Group] | | | | |
| | | | | &gt:gi\|113631669\|dbj\|BAF25350.1\| | | | | |
| | | | | Os09g0470500 [Oryza sativa Japonica Group] | | | | |
| | | | | &gt:gi\|21 8202304\|gb\|EEC84731.1\| | | | | |
| | | | | hypothetical protein OsI_31718 | | | | |
| | | | | [Oryza sativa Indica Group] | | | | |
| | | BAJ85282 | 326493641 | predicted protein | 0.74359 | Hordeum vulgare subsp. vulgare | 3349 | 4695 |
| | | | | [Hordeum vulgare subsp. vulgare] | | | | |
| | | | | &gt:gi\|326502458\|dbj\|BAJ95292.1\| | | | | |
| | | | | predicted protein | | | | |
| | | | | [Hordeum vulgare subsp. vulgare] | | | | |
| | | | | &gt:gi\|326509779\|dbj\|BAJ87105.1\| | | | | |
| | | | | predicted protein | | | | |
| | | | | [Hordeum vulgare subsp. vulgare] | | | | |
| | 521-542 | XP_002489042 | 253761062 | hypothetical protein SORBIDRAFT_0283s002010 | 1 | Sorghum bicolor | 3350 | 4696 |
| | | | | [Sorghum bicolor] | | | | |
| | | | | &gt:gi\|241947303\|gb\|EES20448.1\| | | | | |
| | | | | hypothetical protein SORBIDRAFT_0283s002010 | | | | |
| | | | | [Sorghum bicolor] | | | | |
| | | ACG35382 | 195627103 | leucine-rich repeat receptor protein | 0.878116 | Zea mays | 3351 | 4697 |
| | | | | kinase EXS precursor [Zea mays] | | | | |
| | | ACG30302 | 195616943 | leucine-rich repeat receptor protein | 0.858726 | Zea mays | 3352 | 4698 |
| | | | | kinase EXS precursor [Zea mays] | | | | |
| | | NP_001053443 | 115459685 | Os04g0540900 [Oryza sativa Japonica Group] | 0.778393 | Oryza sativa Japonica Group | 3353 | 4699 |
| | | | | &gt:gi\|38344983\|emb\|CAE02789.2\| | | | | |
| | | | | OSJNBa0011L07.13 [Oryza sativa Japonica Group] | | | | |
| | | | | &gt:gi\|113565014\|dbj\|BAF15357.1\| | | | | |
| | | | | Os04g0540900 [Oryza sativa Japonica Group] | | | | |
| | | | | &gt:gi\|116310384\|emb\|CAH67395.1\| | | | | |
| | | | | H0115B09.7 [Oryza sativa Indica Group] | | | | |
| | | | | &gt:gi\|125549190\|gb\|EAY95012.1\| | | | | |
| | | | | hypothetical protein OsI_16820 | | | | |
| | | | | [Oryza sativa Indica Group] | | | | |
| | | | | &gt:gi\|125591143\|gb\|EAZ31493.1\| | | | | |
| | | | | hypothetical protein OsJ_15629 | | | | |
| | | | | [Oryza sativa Japonica Group] | | | | |
| | | | | &gt:gi\|215694759\|dbj\|BAG89950.1\| | | | | |
| | | | | unnamed protein product | | | | |
| | | | | [Oryza sativa Japonica Group] | | | | |
| | | BAJ85198 | 326493473 | predicted protein | 0.731302 | Hordeum vulgare subsp. vulgare | 3354 | 4700 |
| | | | | [Hordeum vulgare subsp. vulgare] | | | | |
| | | | | &gt:gi\|326516176\|dbj\|BAJ88111.1\| | | | | |
| | | | | predicted protein | | | | |
| | | | | [Hordeum vulgare subsp. vulgare] | | | | |
| | 129-150 | XP_002448827 | 242077781 | hypothetical protein SORBIDRAFT_06g033930 | 1 | Sorghum bicolor | 3355 | 4701 |
| | | | | [Sorghum bicolor] | | | | |
| | | | | &gt:gi\|241940010\|gb\|EES13155.1\| | | | | |
| | | | | hypothetical protein SORBIDRAFT_06g033930 | | | | |
| | | | | [Sorghum bicolor] | | | | |
| | | NP_001169688 | 293332518 | hypothetical protein LOC100383569 | 0.972477 | Zea mays | 3356 | 4702 |
| | | | | [Zea mays] | | | | |
| | | | | &gt:gi\|224030901\|gb\|ACN34526.1\| | | | | |
| | | | | unknown [Zea mays] | | | | |
| | | NP_001061552 | 115475911 | Os08g0326600 [Oryza sativa Japonica Group] | 0.902752 | Oryza sativa Japonica Group | 3357 | 4703 |
| | | | | &gt:gi\|24414065\|dbj\|BAC22314.1\| | | | | |
| | | | | putative GMP synthetase | | | | |
| | | | | [Oryza sativa Japonica Group] | | | | |
| | | | | &gt:gi\|113623521\|dbj\|BAF23466.1\| | | | | |
| | | | | Os08g0326600 [Oryza sativa Japonica Group] | | | | |
| | | | | &gt:gi\|215694477\|dbj\|BAG89422.1\| | | | | |
| | | | | unnamed protein product | | | | |
| | | | | [Oryza sativa Japonica Group] | | | | |
| | | BAK00536 | 326524304 | predicted protein | 0.880734 | Hordeum vulgare subsp. vulgare | 3358 | 4704 |
| | | | | [Hordeum vulgare subsp. vulgare] | | | | |
| | | XP_002274590 | 225426433 | PREDICTED: hypothetical protein [Vitis vinifera] | 0.746789 | Vitis vinifera | 3359 | 4705 |
| | | XP_002304500 | 224074940 | predicted protein [Populus trichocarpa] | 0.73945 | Populus trichocarpa | 3360 | 4706 |
| | | | | &gt:gi\|222841932\|gb\|EEE79479.1\| | | | | |
| | | | | predicted protein [Populus trichocarpa] | | | | |
| | | XP_002886394 | 297837024 | hypothetical protein ARALYDRAFT_893078 | 0.733945 | Arabidopsis lyrata subsp. lyrata | 3361 | 4707 |
| | | | | [Arabidopsis lyrata subsp. lyrata] | | | | |
| | | | | &gt:gi\|297332235\|gb\|EFH62653.1\| | | | | |
| | | | | hypothetical protein ARALYDRAFT_893078 | | | | |
| | | | | [Arabidopsis lyrata subsp. lyrata] | | | | |
| | | NP_176553 | 42562914 | GMP-synthase-C and glutamine amidotransferase | 0.733945 | Arabidopsis thaliana | 3362 | 4708 |
| | | | | domain-containing protein | | | | |
| | | | | [Arabidopsis thaliana] | | | | |
| | | | | &gt:gi\|12324937\|gb\|AAG52416.1\|AC011622_4 | | | | |
| | | | | GMP synthase; 61700-64653 | | | | |
| | | | | [Arabidopsis thaliana] | | | | |
| | | | | &gt:gi\|56381989\|gb\|AAV85713.1\| | | | | |
| | | | | At1g63660 [Arabidopsis thaliana] | | | | |
| | | | | &gt:gi\|332196006\|gb\|AEE34127.1\| | | | | |
| | | | | GMP-synthase-C and glutamine amidotransferase | | | | |
| | | | | domain-containing protein | | | | |
| | | | | [Arabidopsis thaliana] | | | | |
| | | AAO42053 | 28393248 | putative GMP synthase | 0.73211 | Arabidopsis thaliana | 3363 | 4709 |
| | | | | [Arabidopsis thaliana] | | | | |
| | 194-215 | XP_002467027 | 242039264 | hypothetical protein SORBIDRAFT_01g018490 | 1 | Sorghum bicolor | 3364 | 4710 |
| | | | | [Sorghum bicolor] | | | | |
| | | | | &gt:gi\|241920881\|gb\|EER94025.1\| | | | | |
| | | | | hypothetical protein SORBIDRAFT_01g018490 | | | | |
| | | | | [Sorghum bicolor] | | | | |
| | | NP_001149395 | 226532849 | LOC100283021 [Zea mays] | 0.857778 | Zea mays | 3365 | 4711 |
| | | | | &gt:gi\|195626940\|gb\|ACG35300.1\| | | | | |
| | | | | secretory protein [Zea mays] | | | | |
| | | ABV22582 | 157093711 | PR17c precursor | 0.808889 | Hordeum vulgare subsp. vulgare | 3366 | 4712 |
| | | | | [Hordeum vulgare subsp. vulgare] | | | | |
| | | | | &gt:gi\|157093714\|gb\|ABV22583.1\| | | | | |
| | | | | PR17c precursor | | | | |
| | | | | [Hordeum vulgare subsp. vulgare] | | | | |
| | | | | &gt:gi\|157093720\|gb\|ABV22586.1\| | | | | |
| | | | | PR17c precursor | | | | |
| | | | | [Hordeum vulgare subsp. vulgare] | | | | |
| | | CAA74594 | 2266665 | hypothetical protein [Hordeum vulgare] | 0.808889 | Hordeum vulgare | 3367 | 4713 |
| | | | | &gt:gi\|326494904\|dbj\|BAJ85547.1\| | | | | |
| | | | | predicted protein | | | | |
| | | | | [Hordeum vulgare subsp. vulgare] | | | | |
| | | | | &gt:gi\|326514206\|dbj\|BAJ92253.1\| | | | | |
| | | | | predicted protein | | | | |
| | | | | [Hordeum vulgare subsp. vulgare] | | | | |
| | | XP_002467026 | 242039262 | hypothetical protein SORBIDRAFT_01g018480 | 0.733333 | Sorghum bicolor | 3368 | 4714 |
| | | | | [Sorghum bicolor] | | | | |
| | | | | &gt:gi\|241920880\|gb\|EER94024.1\| | | | | |
| | | | | hypothetical protein SORBIDRAFT_01g018480 | | | | |
| | | | | [Sorghum bicolor] | | | | |
| | | AAD46133 | 5669007 | secretory protein [Triticum aestivum] | 0.791111 | Triticum aestivum | 3369 | 4715 |
| | | EEC67221 | 54362548 | hypothetical protein OsI_34133 | 0.702222 | Oryza sativa Indica Group | 3370 | |
| | | | | [Oryza sativa Indica Group] | | | | |
| | 1270-1291 | ACN25803 | 223943438 | unknown [Zea mays] | 1 | Zea mays | 3371 | 4716 |
| | | NP_001144623 | 226502924 | hypothetical protein LOC100277639 [Zea mays] | 0.975741 | Zea mays | 3372 | 4717 |
| | | | | &gt:gi\|195644748\|gb\|ACG41842.1\| | | | | |
| | | | | hypothetical protein [Zea mays] | | | | |
| | | XP_002456637 | 242054982 | hypothetical protein SORBIDRAFT_03g039920 | 0.830189 | Sorghum bicolor | 3373 | 4718 |
| | | | | [Sorghum bicolor] | | | | |
| | | | | &gt:gi\|241928612\|gb\|EES01757.1\| | | | | |
| | | | | hypothetical protein SORBIDRAFT_03g039920 | | | | |
| | | | | [Sorghum bicolor] | | | | |
| | | ACL53226 | 219885702 | unknown [Zea mays] | 0.762803 | Zea mays | 3374 | 4719 |
| | 1323-1344 | XP_002450912 | 242071270 | hypothetical protein SORBIDRAFT_05g021030 | 1 | Sorghum bicolor | 3375 | 4720 |
| | | | | [Sorghum bicolor] | | | | |
| | | | | &gt:gi\|241936755\|gb\|EES09900.1\| | | | | |
| | | | | hypothetical protein SORBIDRAFT_05g021030 | | | | |
| | | | | [Sorghum bicolor] | | | | |
| | | NP_001146157 | 226497549 | hypothetical protein LOC100279726 [Zea mays] | 0.936995 | Zea mays | 3376 | 4721 |
| | | | | &gt:gi\|224030419\|gb\|ACN34285.1\| | | | | |
| | | | | unknown [Zea mays] | | | | |
| | | NP_001068068 | 297611975 | Os11g0547000 [Oryza sativa Japonica Group] | 0.851373 | Oryza sativa Japonica Group | 3377 | 4722 |
| | | | | &gt:gi\|110832734\|sp\|Q2R2W1.2\|ADO3_ORYSJ | | | | |
| | | | | RecName: Full = Adagio-like protein 3 | | | | |
| | | | | &gt:gi\|108864481\|gb\|ABA94231.2\| Adagio 3, | | | | |
| | | | | putative, expressed [Oryza sativa Japonica Group] | | | | |
| | | | | &gt:gi\|255680164\|dbj\|BAF28431.2\| | | | | |
| | | | | Os11g0547000 | | | | |
| | | | | [Oryza sativa Japonica Group] | | | | |
| | | EAZ18680 | 54398660 | hypothetical protein OsJ_34201 | 0.849758 | Oryza sativa Japonica Group | 3378 | |
| | | | | [Oryza sativa Japonica Group] | | | | |
| | | BAJ95049 | 326500765 | predicted protein | 0.849758 | Hordeum vulgare subsp. vulgare | 3379 | 4723 |
| | | | | [Hordeum vulgare subsp. vulgare] | | | | |
| | | ABL11478 | 118767204 | FKF1 protein [Triticum aestivum] | 0.812601 | Triticum aestivum | 3380 | 4724 |
| | | ACL53371 | 219885992 | unknown [Zea mays] | 0.781906 | Zea mays | 3381 | 4725 |
| | | | | &gt:gi\|223942805\|gb\|ACN25486.1\| | | | | |
| | | | | unknown [Zea mays] | | | | |
| | | EAY81253 | 54362548 | hypothetical protein OsI_36431 | 0.767367 | Oryza sativa Indica Group | 3382 | |
| | | | | [Oryza sativa Indica Group] | | | | |
| | | ABG22521 | 108863896 | Adagio 3, putative, expressed | 0.767367 | Oryza sativa Japonica Group | 3383 | 4726 |
| | | | | [Oryza sativa Japonica Group] | | | | |
| | | ABD28287 | 87138100 | circadian clock-associated FKF1 | 0.710824 | Glycine max | 3384 | 4727 |
| | | | | [Glycine max] | | | | |
| | 196-217 | ACO72994 | 226701035 | DRE-binding protein 3 [Zea mays] | 1 | Zea mays | 3385 | 4728 |
| | | NP_001147172 | 226504965 | sbCBF6 [Zea mays] | 0.911765 | Zea mays | 3386 | 4729 |
| | | | | &gt:gi\|195607972\|gb\|ACG25816.1\| | | | | |
| | | | | sbCBF6 [Zea mays] | | | | |
| | | XP_002462691 | 242049893 | hypothetical protein SORBIDRAFT_02g030320 | 0.831933 | Sorghum bicolor | 3387 | 4730 |
| | | | | [Sorghum bicolor] | | | | |
| | | | | &gt:gi\|241926068\|gb\|EER99212.1\| | | | | |
| | | | | hypothetical protein SORBIDRAFT_02g030320 | | | | |
| | | | | [Sorghum bicolor] | | | | |
| | | NP_001063712 | 115480236 | Os09g0522200 [Oryza sativa Japonica Group] | 0.739496 | Oryza sativa Japonica Group | 3388 | 4731 |
| | | | | &gt:gi\|75253216\|sp\|Q64MA1.1\|DRE1A_ORYSJ | | | | |
| | | | | RecName: Full = Dehydration-responsive element- | | | | |
| | | | | binding protein 1A; Short = Protein DREB1A; | | | | |
| | | | | AltName: Full = Protein C-repeat-binding factor 3; | | | | |
| | | | | Short = rCBF3 | | | | |
| | | | | &gt:gi\|22594969\|gb\|AAN02486.1\|AF300970_1 | | | | |
| | | | | DRE-binding protein 1A | | | | |
| | | | | [Oryza sativa] | | | | |
| | | | | &gt:gi\|52075594\|dbj\|BAD46704.1\| | | | | |
| | | | | DRE-binding protein 1A | | | | |
| | | | | [Oryza sativa Japonica Group] | | | | |
| | | | | &gt:gi\|113631945\|dbj\|BAF25626.1\| | | | | |
| | | | | Os09g0522200 [Oryza sativa Japonica Group] | | | | |
| | | A2Z389 | | RecName: Full = Dehydration-responsive element- | 0.735294 | Oryza sativa | 3389 | |
| | | | | binding protein 1A; Short = Protein DREB1A; | | | | |
| | | | | AltName: Full = Protein C-repeat-binding factor 3; | | | | |
| | | | | Short = rCBF3 | | | | |
| | | | | &gt:gi\|33321848\|gb\|AAQ06658.1\| | | | | |
| | | | | apetala2 domain-containing CBF1-like protein | | | | |
| | | | | [Oryza sativa] | | | | |
| | | | | &gt:gi\|125564420\|gb\|EAZ09800.1\| | | | | |
| | | | | hypothetical protein OsI_32087 | | | | |
| | | | | [Oryza sativa Indica Group] | | | | |
| | | AAQ23983 | 33637697 | transcription factor RCBF3 | 0.735294 | Oryza sativa | 3390 | 4732 |
| | | | | [Oryza sativa] | | | | |
| | | ABG73450 | 110430645 | DREBlb [Oryza brachyantha] | 0.705882 | Oryza brachyantha | 3391 | |
| | | XP_002462692 | 242049895 | hypothetical protein SORBIDRAFT_02g030330 | 0.714286 | Sorghum bicolor | 3392 | 4733 |
| | | | | [Sorghum bicolor] | | | | |
| | | | | &gt:gi\|60593391\|gb\|AAX28960.1\| | | | | |
| | | | | SbCBF6 [Sorghum bicolor] | | | | |
| | | | | &gt:gi\|241926069\|gb\|EER99213.1\| | | | | |
| | | | | hypothetical protein SORBIDRAFT_02g030330 | | | | |
| | | | | [Sorghum bicolor] | | | | |
| | 1246-1267 | XP_002463252 | 242051015 | hypothetical protein SORBIDRAFT_02g040610 | 1 | Sorghum bicolor | 3393 | 4734 |
| | | | | [Sorghum bicolor] | | | | |
| | | | | &gt:gi\|241926629\|gb\|EER99773.1\| | | | | |
| | | | | hypothetical protein SORBIDRAFT_02g040610 | | | | |
| | | | | [Sorghum bicolor] | | | | |
| | | ACF78838 | 194689507 | unknown [Zea mays] | 0.958678 | Zea mays | 3394 | 4735 |
| | | | | &gt:gi\|238009848\|gb\|ACR35959.1\| | | | | |
| | | | | unknown [Zea mays] | | | | |
| | | NP_001105608 | 162458296 | pyruvate dehydrogenase (lipoamide) kinase2 | 0.955923 | Zea mays | 3395 | 4736 |
| | | | | [Zea mays] | | | | |
| | | | | &gt:gi\|3695005\|gb\|AAC63962.1\| | | | | |
| | | | | pyruvate dehydrogenase kinase isoform 2 | | | | |
| | | | | [Zea mays] | | | | |
| | | NP_001060401 | 115473604 | Os07g0637300 [Oryza sativa Japonica Group] | 0.898072 | Oryza sativa Japonica Group | 3396 | 4737 |
| | | | | &gt:gi\|12829952\|gb\|AAK01947.1\| | | | | |
| | | | | pyruvate dehydrogenase kinase 1 | | | | |
| | | | | [Oryza sativa Indica Group] | | | | |
| | | | | &gt:gi\|23237829\|dbj\|BAC16404.1\| | | | | |
| | | | | pyruvate dehydrogenase kinase 1 | | | | |
| | | | | [Oryza sativa Japonica Group] | | | | |
| | | | | &gt:gi\|60499792\|gb\|AAX22104.1\| | | | | |
| | | | | pyruvate dehydrogenase kinase | | | | |
| | | | | [Oryza sativa Indica Group] | | | | |
| | | | | &gt:gi\|60499794\|gb\|AAX22105.1\| | | | | |
| | | | | pyruvate dehydrogenase kinase | | | | |
| | | | | [Oryza sativa Indica Group] | | | | |
| | | | | &gt:gi\|113611937\|dbj\|BAF22315.1\| | | | | |
| | | | | Os07g0637300 [Oryza sativa Japonica Group] | | | | |
| | | EEC82518 | 54362548 | hypothetical protein OsI_27023 | 0.898072 | Oryza sativa Indica Group | 3397 | |
| | | | | [Oryza sativa Indica Group] | | | | |
| | | EEE67664 | 54398660 | hypothetical protein OsJ_25286 | 0.895317 | Oryza sativa Japonica Group | 3398 | |
| | | | | [Oryza sativa Japonica Group] | | | | |
| | | BAJ85210 | 326493497 | predicted protein | 0.878788 | Hordeum vulgare subsp. vulgare | 3399 | 4738 |
| | | | | [Hordeum vulgare subsp. vulgare] | | | | |
| | | XP_002467803 | 242040816 | hypothetical protein SORBIDRAFT_01g034390 | 0.812672 | Sorghum bicolor | 3400 | 4739 |
| | | | | [Sorghum bicolor] | | | | |
| | | | | &gt:gi\|241921657\|gb\|EER94801.1\| | | | | |
| | | | | hypothetical protein SORBIDRAFT_01g034390 | | | | |
| | | | | [Sorghum bicolor] | | | | |
| | | NP_001104897 | 162459440 | pyruvate dehydrogenase (lipoamide) kinase1 | 0.807163 | Zea mays | 3401 | 4740 |
| | | | | [Zea mays] | | | | |
| | | | | &gt:gi\|3746431\|gb\|AAC63961.1\| | | | | |
| | | | | pyruvate dehydrogenase kinase isoform 1 | | | | |
| | | | | [Zea mays] | | | | |
| | | | | &gt:gi\|219885093\|gb\|ACL52921.1\| | | | | |
| | | | | unknown [Zea mays] | | | | |
| | | NP_001132485 | 212721389 | hypothetical protein LOC100193944 | 0.801653 | Zea mays | 3402 | 4741 |
| | | | | [Zea mays] | | | | |
| | | | | &gt:gi\|194694512\|gb\|ACF81340.1\| | | | | |
| | | | | unknown [Zea mays] | | | | |
| Predicted | 829-850 | XP_002441194 | 242090722 | hypothetical protein SORBIDRAFT_09g022010 | 1 | Sorghum bicolor | 3403 | 4742 |
| zma | | | | [Sorghum bicolor] | | | | |
| mir | | | | &gt:gi\|241946479\|gb\|EES19624.1\| | | | | |
| 49718 | | | | hypothetical protein SORBIDRAFT_09g022010 | | | | |
| | | | | [Sorghum bicolor] | | | | |
| | | NP_001144777 | 226503764 | hypothetical protein LOC100277843 | 0.879684 | Zea mays | 3404 | 4743 |
| | | | | [Zea mays] | | | | |
| | | | | &gt:gi\|195646864\|gb\|ACG42900.1\| | | | | |
| | | | | hypothetical protein [Zea mays] | | | | |
| | | NP_001055695 | 115464190 | Os05g0447700 [Oryza sativa Japonica Group] | 0.783037 | Oryza sativa Japonica Group | 3405 | 4744 |
| | | | | &gt:gi\|51854380\|gb\|AAU10760.1\| unknown | | | | |
| | | | | protein [Oryza sativa Japonica Group] | | | | |
| | | | | &gt:gi\|113579246\|dbj\|BAF17609.1\| | | | | |
| | | | | Os05g0447700 [Oryza sativa Japonica Group] | | | | |
| | | | | &gt:gi\|215701012\|dbj\|BAG92436.1\| | | | | |
| | | | | unnamed protein product | | | | |
| | | | | [Oryza sativa Japonica Group] | | | | |
| | | | | &gt:gi\|215701421\|dbj\|BAG92845.1\| | | | | |
| | | | | unnamed protein product | | | | |
| | | | | [Oryza sativa Japonica Group] | | | | |
| | | | | &gt:gi\|222631775\|gb\|EEE63907.1\| | | | | |
| | | | | hypothetical protein OsJ_18732 | | | | |
| | | | | [Oryza sativa Japonica Group] | | | | |
| | | EAY98243 | 54362548 | hypothetical protein OsI_20153 | 0.781065 | Oryza sativa Indica Group | 3406 | |
| | | | | [Oryza sativa Indica Group] | | | | |
| | | BAJ91869 | 326511042 | predicted protein | 0.700197 | Hordeum vulgare subsp. vulgare | 3407 | 4745 |
| | | | | [Hordeum vulgare subsp. vulgare] | | | | |
| | 2065-2086 | BAD02277 | 38488589 | vacuolar proton pyrophosphatase | 1 | Oryza sativa Japonica Group | 3408 | |
| | | | | [Oryza sativa Japonica Group] | | | | |
| | | BAD25066 | 38488637 | putative inorganic diphosphatase | 0.998701 | Oryza sativa Japonica Group | 3409 | |
| | | | | [Oryza sativa Japonica Group] | | | | |
| | | | | &gt:gi\|222622322\|gb\|EEE56454.1\| | | | | |
| | | | | hypothetical protein OsJ_05651 | | | | |
| | | | | [Oryza sativa Japonica Group] | | | | |
| | | XP_002453419 | 242064259 | hypothetical protein SORBIDRAFT_04g005710 | 0.961039 | Sorghum bicolor | 3410 | 4746 |
| | | | | [Sorghum bicolor] | | | | |
| | | | | &gt:gi\|241933250\|gb\|EES06395.1\| | | | | |
| | | | | hypothetical protein SORBIDRAFT_04g005710 | | | | |
| | | | | [Sorghum bicolor] | | | | |
| | | CAG29370 | 57161867 | vacuolar H+-translocating inorganic | 0.950649 | Zea mays | 3411 | |
| | | | | pyrophosphatase [Zea mays] | | | | |
| | | BAJ91598 | 326509362 | predicted protein | 0.92987 | Hordeum vulgare subsp. vulgare | 3412 | 4747 |
| | | | | [Hordeum vulgare subsp. vulgare] | | | | |
| | | NP_001058184 | 115469169 | Os06g0644200 [Oryza sativa Japonica Group] | 0.937662 | Oryza sativa Japonica Group | 3413 | 4748 |
| | | | | &gt:gi\|51535512\|dbj\|BAD37431.1\| | | | | |
| | | | | inorganic diphosphatase, H+-translocating, vacuolar | | | | |
| | | | | membrane [Oryza sativa Japonica Group] | | | | |
| | | | | &gt:gi\|113596224\|dbj\|BAF20098.1\| | | | | |
| | | | | Os06g0644200 [Oryza sativa Japonica Group] | | | | |
| | | | | &gt:gi\|215704675\|dbj\|BAG94303.1\| | | | | |
| | | | | unnamed protein product | | | | |
| | | | | [Oryza sativa Japonica Group] | | | | |
| | | BAA08232 | 1747293 | vacuolar H+-pyrophosphatase | 0.936364 | Oryza sativa (Japonica cultivar-group)] | 3414 | 4749 |
| | | | | [Oryza sativa (Japonica cultivar-group)] | | | | |
| | | BAA31523 | 3298473 | Ovp1 [Oryza sativa] | 0.935065 | Oryza sativa | 3415 | 4750 |
| | | BAB18681 | 11527560 | vacuolar proton-inorganic pyrophosphatase | 0.925974 | Hordeum vulgare subsp. vulgare | 3416 | 4751 |
| | | | | [Hordeum vulgare subsp. vulgare] | | | | |
| | | EEE66103 | 54398660 | hypothetical protein OsJ_22136 | 0.935065 | Oryza sativa Japonica Group | 3417 | |
| | | | | [Oryza sativa Japonica Group] | | | | |
| Predicted | 749-769 | ACN36483 | 224034814 | unknown [Zea mays] | 1 | Zea mays | 3418 | 4752 |
| zma | | | | | | | | |
| mir | | | | | | | | |
| 50085 | | | | | | | | |
| | | XP_002464347 | 242033904 | hypothetical protein SORBIDRAFT_01g016710 | 0.753709 | Sorghum bicolor | 3419 | 4753 |
| | | | | [Sorghum bicolor] | | | | |
| | | | | &gt:gi\|241918201\|gb\|EER91345.1\| | | | | |
| | | | | hypothetical protein SORBIDRAFT_01g016710 | | | | |
| | | | | [Sorghum bicolor] | | | | |
| | 772-792 | XP_002441965 | 242083079 | hypothetical protein SORBIDRAFT_08g005660 | 1 | Sorghum bicolor | 3420 | 4754 |
| | | | | [Sorghum bicolor] | | | | |
| | | | | &gt:gi\|241942658\|gb\|EES15803.1\| | | | | |
| | | | | hypothetical protein SORBIDRAFT_08g005660 | | | | |
| | | | | [Sorghum bicolor] | | | | |
| | | NP_001147944 | 226491563 | AAP6 [Zea mays] | 0.887734 | Zea mays | 3421 | 4755 |
| | | | | &gt:gi\|195614738\|gb\|ACG29199.1\| | | | | |
| | | | | AAP6 [Zea mays] | | | | |
| | | ACF82748 | 194697327 | unknown [Zea mays] | 0.881497 | Zea mays | 3422 | 4756 |
| | | NP_001066353 | 115487731 | Os12g0194900 [Oryza sativa Japonica Group] | 0.881497 | Oryza sativa Japonica Group | 3423 | 4757 |
| | | | | &gt:gi\|108862289\|gb\|ABA96080.2\| | | | | |
| | | | | amino acid permease I, putative, expressed | | | | |
| | | | | [Oryza sativa Japonica Group] | | | | |
| | | | | &gt:gi\|113648860\|dbj\|BAF29372.1\| | | | | |
| | | | | Os12g0194900 [Oryza sativa Japonica Group] | | | | |
| | | | | &gt:gi\|125536049\|gb\|EAY82537.1\| | | | | |
| | | | | hypothetical protein OsI_37760 | | | | |
| | | | | [Oryza sativa Indica Group] | | | | |
| | 113-133 | EEC74142 | 54362548 | hypothetical protein OsI_09216 | 1 | Oryza sativa Indica Group | 3424 | |
| | | | | [Oryza sativa Indica Group] | | | | |
| | | NP_001152051 | 226502027 | serine esterase family protein | 0.848765 | Zea mays | 3425 | 4758 |
| | | | | [Zea mays] | | | | |
| | | | | &gt:gi\|195652153\|gb\|ACG45544.1\| | | | | |
| | | | | serine esterase family protein | | | | |
| | | | | [Zea mays] | | | | |
| | | ACF80204 | 194692239 | unknown [Zea mays] | 0.712963 | Zea mays | 3426 | 4759 |
| | | XP_002452943 | 242063307 | hypothetical protein SORBIDRAFT_04g035350 | 0.728395 | Sorghum bicolor | 3427 | 4760 |
| | | | | [Sorghum bicolor] | | | | |
| | | | | &gt:gi\|241932774\|gb\|EES05919.1\| | | | | |
| | | | | hypothetical protein SORBIDRAFT_04g035350 | | | | |
| | | | | [Sorghum bicolor] | | | | |
| | 200-220 | XP_002462815 | 242050141 | hypothetical protein SORBIDRAFT_02g032420 | 1 | Sorghum bicolor | 3428 | 4761 |
| | | | | [Sorghum bicolor] | | | | |
| | | | | &gt:gi\|241926192\|gb\|EER99336.1\| | | | | |
| | | | | hypothetical protein SORBIDRAFT_02g032420 | | | | |
| | | | | [Sorghum bicolor] | | | | |
| | | EEC85037 | 54362548 | hypothetical protein OsI_32346 | 0.964072 | Oryza sativa Japonica Group | 3429 | |
| | | | | [Oryza sativa Indica Group] | | | | |
| | | | | &gt:gi\|222642071\|gb\|EEE70203.1\| | | | | |
| | | | | hypothetical protein OsJ_30293 | | | | |
| | | | | [Oryza sativa Japonica Group] | | | | |
| | | NP_001142116 | 226502757 | hypothetical protein LOC100274280 | 0.976048 | Zea mays | 3430 | 4762 |
| | | | | [Zea mays] | | | | |
| | | | | &gt:gi\|194704262\|gb\|ACF86215.1\| | | | | |
| | | | | unknown [Zea mays] | | | | |
| | | | | &gt:gi\|194707182\|gb\|ACF87675.1\| | | | | |
| | | | | unknown [Zea mays] | | | | |
| | | BAJ85748 | 326495303 | predicted protein | 0.952096 | Hordeum vulgare subsp. vulgare | 3431 | 4763 |
| | | | | [Hordeum vulgare subsp. vulgare] | | | | |
| | | | | &gt:gi\|326508052\|dbj\|BAJ86769.1\| | | | | |
| | | | | predicted protein | | | | |
| | | | | [Hordeum vulgare subsp. vulgare] | | | | |
| | | | | &gt:gi\|326524422\|dbj\|BAK00594.1\| | | | | |
| | | | | predicted protein | | | | |
| | | | | [Hordeum vulgare subsp. vulgare] | | | | |
| | | ACU13649 | 255626608 | unknown [Glycine max] | 0.820359 | Glycine max | 3432 | 4764 |
| | | ACU14908 | 255629126 | unknown [Glycine max] | 0.820359 | Glycine max | 3433 | 4765 |
| | | XP_002284866 | 225451010 | PREDICTED: hypothetical protein | 0.790419 | Vitis vinifera | 3434 | 4766 |
| | | | | [Vitis vinifera] | | | | |
| | | ACG47475 | 195656014 | hypothetical protein [Zea mays] | 0.874251 | Zea mays | 3435 | 4767 |
| | | XP_002512352 | 255542577 | microsomal signal peptidase 23 kD subunit, putative | 0.784431 | Ricinus communis | 3436 | 4768 |
| | | | | [Ricinus communis] | | | | |
| | | | | &gt:gi\|223548313\|gb\|EEF49804.1\| | | | | |
| | | | | microsomal signal peptidase 23 kD subunit, putative | | | | |
| | | | | [Ricinus communis] | | | | |
| | 946-966 | XP_002452943 | 242063307 | hypothetical protein SORBIDRAFT_04g035350 | 1 | Sorghum bicolor | 3437 | 4769 |
| | | | | [Sorghum bicolor] | | | | |
| | | | | &gt:gi\|241932774\|gb\|EES05919.1\| | | | | |
| | | | | hypothetical protein SORBIDRAFT_04g035350 | | | | |
| | | | | [Sorghum bicolor] | | | | |
| | | ACF88103 | 194708037 | unknown [Zea mays] | 0.9 | Zea mays | 3438 | 4770 |
| | | NP_001048339 | 115449118 | Os02g0787100 [Oryza sativa Japonica Group] | 0.757895 | Oryza sativa Japonica Group | 3439 | 4771 |
| | | | | &gt:gi\|47497166\|dbj\|BAD19214.1\| | | | | |
| | | | | hypothetical protein [Oryza sativa Japonica Group] | | | | |
| | | | | &gt:gi\|47497751\|dbj\|BAD19851.1\| | | | | |
| | | | | hypothetical protein [Oryza sativa Japonica Group] | | | | |
| | | | | &gt:gi\|113537870\|dbj\|BAF10253.1\| | | | | |
| | | | | Os02g0787100 [Oryza sativa Japonica Group] | | | | |
| | | BAJ88501 | 326522910 | predicted protein | 0.7 | Hordeum vulgare subsp. vulgare | 3440 | 4772 |
| | | | | [Hordeum vulgare subsp. vulgare] | | | | |
| | 231-251 | NP_001150320 | 226502818 | signal peptidase complex subunit 3 | 1 | Zea mays | 3441 | 4773 |
| | | | | [Zea mays] | | | | |
| | | | | &gt:gi\|194695862\|gb\|ACF82015.1\| | | | | |
| | | | | unknown [Zea mays] | | | | |
| | | | | &gt:gi\|195638350\|gb\|ACG38643.1\| | | | | |
| | | | | signal peptidase complex subunit 3 | | | | |
| | | | | [Zea mays] | | | | |
| | | ACG47475 | 195656014 | hypothetical protein [Zea mays] | 0.862275 | Zea mays | 3442 | 4774 |
| | | AAO45754 | 307135766 | signal peptidase protein-like protein | 0.778443 | Cucumis melo subsp. melo | 3443 | 4775 |
| | | | | [Cucumis melo subsp. melo] | | | | |
| | 1001-1021 | XP_002436903 | 242092825 | hypothetical protein SORBIDRAFT_10g010840 | 1 | Sorghum bicolor | 3444 | 4776 |
| | | | | [Sorghum bicolor] | | | | |
| | | | | &gt:gi\|241915126\|gb\|EER88270.1\| | | | | |
| | | | | hypothetical protein SORBIDRAFT_10g010840 | | | | |
| | | | | [Sorghum bicolor] | | | | |
| | | NP_001130415 | 212274954 | hypothetical protein LOC100191511 | 0.954128 | Zea mays | 3445 | 4777 |
| | | | | [Zea mays] | | | | |
| | | | | &gt:gi\|194689060\|gb\|ACF78614.1\| | | | | |
| | | | | unknown [Zea mays] | | | | |
| | | | | &gt:gi\|223942719\|gb\|ACN25443.1\| | | | | |
| | | | | unknown [Zea mays] | | | | |
| | | | | &gt:gi\|224029573\|gb\|ACN33862.1\| | | | | |
| | | | | unknown [Zea mays] | | | | |
| | | NP_001057438 | 115467677 | Os06g0297700 [Oryza sativa Japonica Group] | 0.821101 | Oryza sativa Japonica Group | 3446 | 4778 |
| | | | | &gt:gi\|53791981\|dbj\|BAD54434.1\| | | | | |
| | | | | unknown protein | | | | |
| | | | | [Oryza sativa Japonica Group] | | | | |
| | | | | &gt:gi\|113595478\|dbj\|BAF19352.1\| | | | | |
| | | | | Os06g0297700 | | | | |
| | | | | [Oryza sativa Japonica Group] | | | | |
| | | BAK05539 | 326487733 | predicted protein | 0.80581 | Hordeum vulgare subsp. vulgare | 3447 | 4779 |
| | | | | [Hordeum vulgare subsp. vulgare] | | | | |
| | 537-557 | XP_002451778 | 242060977 | hypothetical protein SORBIDRAFT_04g007700 | 1 | Sorghum bicolor | 3448 | 4780 |
| | | | | [Sorghum bicolor] | | | | |
| | | | | &gt:gi\|241931609\|gb\|EES04754.1\| | | | | |
| | | | | hypothetical protein SORBIDRAFT_04g007700 | | | | |
| | | | | [Sorghum bicolor] | | | | |
| | | NP_001150860 | 226502363 | dihydrolipoamide S-acetyltransferase1 | 0.955473 | Zea mays | 3449 | 4781 |
| | | | | [Zea mays] | | | | |
| | | | | &gt:gi\|195642434\|gb\|ACG40685.1\| | | | | |
| | | | | dihydrolipoyllysine-residue acetyltransferase | | | | |
| | | | | component of pyruvatedehydrogenase complex | | | | |
| | | | | [Zea mays] | | | | |
| | | NP_001104936 | 162458261 | dihydrolipoamide S-acetyltransferase | 0.946197 | Zea mays | 3450 | 4782 |
| | | | | [Zea mays] | | | | |
| | | | | &gt:gi\|5669871\|gb\|AAD46491.1\|AF135014_1 | | | | |
| | | | | dihydrolipoamide S-acetyltransferase | | | | |
| | | | | [Zea mays] | | | | |
| | | NP_001059445 | 115471692 | Os07g0410100 [Oryza sativa Japonica Group] | 0.834879 | Oryza sativa Japonica Group | 3451 | 4783 |
| | | | | &gt:gi\|33354212\|dbj\|BAC81178.1\| | | | | |
| | | | | unknown protein | | | | |
| | | | | [Oryza sativa Japonica Group] | | | | |
| | | | | &gt:gi\|50510197\|dbj\|BAD31326.1\| | | | | |
| | | | | unknown protein | | | | |
| | | | | [Oryza sativa Japonica Group] | | | | |
| | | | | &gt:gi\|113610981\|dbj\|BAF21359.1\| | | | | |
| | | | | Os07g0410100 | | | | |
| | | | | [Oryza sativa Japonica Group] | | | | |
| | | | | &gt:gi\|215678515\|dbj\|BAG92170.1\| | | | | |
| | | | | unnamed protein | | | | |
| | | | | product [Oryza sativa Japonica Group] | | | | |
| | | EAZ03591 | 54362548 | hypothetical protein OsI_25727 | 0.831169 | Oryza sativa Indica Group | 3452 | |
| | | | | [Oryza sativa Indica Group] | | | | |
| | | NP_001045618 | 115443676 | Os02g0105200 [Oryza sativa Japonica Group] | 0.825603 | Oryza sativa Japonica Group | 3453 | 4784 |
| | | | | &gt:gi\|40363771\|dbj\|BAD06281.1\| | | | | |
| | | | | putative dihydrolipoamide S-acetyltransferase | | | | |
| | | | | [Oryza sativa Japonica Group] | | | | |
| | | | | &gt:gi\|41052549\|dbj\|BAD07541.1\| | | | | |
| | | | | putative dihydrolipoamide S-acetyltransferase | | | | |
| | | | | [Oryza sativa Japonica Group] | | | | |
| | | | | &gt:gi\|50252092\|dbj\|BAD28078.1\| | | | | |
| | | | | putative dihydrolipoamide S-acetyltransferase | | | | |
| | | | | [Oryza sativa Japonica Group] | | | | |
| | | | | &gt:gi\|113535149\|dbj\|BAF07532.1\| | | | | |
| | | | | Os02g0105200 | | | | |
| | | | | [Oryza sativa Japonica Group] | | | | |
| | | | | &gt:gi\|215693370\|dbj\|BAG88752.1\| | | | | |
| | | | | unnamed protein product | | | | |
| | | | | [Oryza sativa Japonica Group] | | | | |
| | | | | &gt:gi\|222622010\|gb\|EEE56142.1\| | | | | |
| | | | | hypothetical protein OsJ_05022 | | | | |
| | | | | [Oryza sativa Japonica Group] | | | | |
| | | EEC72305 | 54362548 | hypothetical protein OsI_05488 | 0.821892 | Oryza sativa Indica Group | 3454 | |
| | | | | [Oryza sativa Indica Group] | | | | |
| | | NP_001056555 | 115465911 | Os06g0105400 [Oryza sativa Japonica Group] | 0.766234 | Oryza sativa Japonica Group | 3455 | 4785 |
| | | | | &gt:gi\|55295833\|dbj\|BAD67701.1\| | | | | |
| | | | | putative dihydrolipoamide S-acetyltransferase | | | | |
| | | | | [Oryza sativa Japonica Group] | | | | |
| | | | | &gt:gi\|113594595\|dbj\|BAF18469.1\| | | | | |
| | | | | Os06g0105400 [Oryza sativa Japonica Group] | | | | |
| | | EEC79834 | 54362548 | hypothetical protein OsI_21298 | 0.756957 | Oryza sativa Indica Group | 3456 | |
| | | | | [Oryza sativa Indica Group] | | | | |
| | | BAJ97318 | 326526602 | predicted protein | 0.766234 | Hordeum vulgare subsp. vulgare | 3457 | 4786 |
| | | | | [Hordeum vulgare subsp. vulgare] | | | | |
| Predicted | 313-331 | NP_001183850 | 308080134 | hypothetical protein LOC100502443 | 1 | Zea mays | 3458 | 4787 |
| zma | | | | [Zea mays] | | | | |
| mir | | | | &gt:gi\|238015018\|gb\|ACR38544.1\| | | | | |
| 50289 | | | | unknown [Zea mays] | | | | |
| | 315-333 | NP_001105340 | 162462750 | ATP synthase subunit beta, mitochondrial precursor | 1 | Zea mays | 3459 | 4788 |
| | | | | [Zea mays] | | | | |
| | | | | &gt:gi\|114420\|sp\|P19023.1\|ATPBM_MAIZE | | | | |
| | | | | RecName: Full = ATP synthase subunit beta, mitochondrial; | | | | |
| | | | | Flags: Precursor | | | | |
| | | | | &gt:gi\|22173\|emb\|CAA38140.1\| | | | | |
| | | | | unnamed protein product | | | | |
| | | | | [Zea mays] | | | | |
| | | | | &gt:gi\|897618\|gb\|AAA70268.1\| | | | | |
| | | | | mitochondrial F-1-ATPase subunit 2 | | | | |
| | | | | [Zea mays] | | | | |
| | | | | &gt:gi\|223943103\|gb\|ACN25635.1\| | | | | |
| | | | | unknown [Zea mays] | | | | |
| | | NP_001151807 | 226493588 | ATP synthase beta chain [Zea mays] | 0.987342 | Zea mays | 3460 | 4789 |
| | | | | &gt:gi\|195614324\|gb\|ACG28992.1\| | | | | |
| | | | | ATP synthase beta chain [Zea mays] | | | | |
| | | | | &gt:gi\|195649793\|gb\|ACG44364.1\| | | | | |
| | | | | ATP synthase beta chain [Zea mays] | | | | |
| | | BAA01372 | 218146 | mitochondrial F1-ATPase beta subunit | 0.949367 | Oryza sativa Japonica Group | 3461 | 4790 |
| | | | | [Oryza sativa Japonica Group] | | | | |
| | | BAJ95263 | 326502399 | predicted protein | 0.933092 | Hordeum vulgare subsp. vulgare | 3462 | 4791 |
| | | | | [Hordeum vulgare subsp. vulgare] | | | | |
| | | | | &gt:gi\|326528609\|dbj\|BAJ97326.1\| | | | | |
| | | | | predicted protein | | | | |
| | | | | [Hordeum vulgare subsp. vulgare] | | | | |
| | | NP_001043900 | 115439240 | Os01g0685800 [Oryza sativa Japonica Group] | 0.94575 | Oryza sativa Japonica Group | 3463 | 4792 |
| | | | | &gt:gi\|113533431\|dbj\|BAF05814.1\| | | | | |
| | | | | Os01g0685800 [Oryza sativa Japonica Group] | | | | |
| | | AAD03392 | 3676295 | mitochondrial ATPase beta subunit | 0.896926 | Nicotiana sylvestris | 3464 | 4793 |
| | | | | [Nicotiana sylvestris] | | | | |
| | | AAD03394 | 3893823 | ATPase beta subunit | 0.898734 | Nicotiana sylvestris | 3465 | 4794 |
| | | | | [Nicotiana sylvestris] | | | | |
| | | AAD03393 | 3893821 | ATPase beta subunit | 0.893309 | Nicotiana sylvestris | 3466 | 4795 |
| | | | | [Nicotiana sylvestris] | | | | |
| | 739-757 | NP_001168893 | 293335318 | hypothetical protein LOC100382698 | 1 | Zea mays | 3467 | 4796 |
| | | | | [Zea mays] | | | | |
| | | | | &gt:gi\|223973523\|gb\|ACN30949.1\| | | | | |
| | | | | unknown [Zea mays] | | | | |
| | | NP_001131845 | 212721699 | hypothetical protein LOC100193221 | 0.991039 | Zea mays | 3468 | 4797 |
| | | | | [Zea mays] | | | | |
| | | | | &gt:gi\|194692700\|gb\|ACF80434.1\| | | | | |
| | | | | unknown [Zea mays] | | | | |
| | | XP_002462395 | 242049301 | hypothetical protein SORBIDRAFT_02g024970 | 0.799283 | Sorghum bicolor | 3469 | 4798 |
| | | | | [Sorghum bicolor] | | | | |
| | | | | &gt:gi\|241925772\|gb\|EER98916.1\| | | | | |
| | | | | hypothetical protein SORBIDRAFT_02g024970 | | | | |
| | | | | [Sorghum bicolor] | | | | |
| | 328-346 | XP_002456162 | 242054032 | hypothetical protein SORBIDRAFT_03g031470 | 1 | Sorghum bicolor | 3470 | 4799 |
| | | | | [Sorghum bicolor] | | | | |
| | | | | &gt:gi\|241928137\|gb\|EES01282.1\| | | | | |
| | | | | hypothetical protein SORBIDRAFT_03g031470 | | | | |
| | | | | [Sorghum bicolor] | | | | |
| | | P17614 | | RecName: Full = ATP synthase subunit beta, | 0.897436 | Nicotiana plumbaginifolia | 3471 | |
| | | | | mitochondrial; Flags: Precursor | | | | |
| | | | | &gt:gi\|19685\|emb\|CAA26620.1\| | | | | |
| | | | | ATP synthase beta subunit | | | | |
| | | | | [Nicotiana plumbaginifolia] | | | | |
| | 417-435 | NP_001056261 | 115465322 | Os05g0553000 [Oryza sativa Japonica Group] | 1 | Oryza sativa Japonica Group | 3472 | 4800 |
| | | | | &gt:gi\|84028177\|sp\|Q01859.2\|ATPBM_ORYSJ | | | | |
| | | | | RecName: Full = ATP synthase subunit beta, | | | | |
| | | | | mitochondrial; Flags: Precursor | | | | |
| | | | | &gt:gi\|49328022\|gb\|AAT58723.1\| | | | | |
| | | | | putative ATP synthase beta chain | | | | |
| | | | | [Oryza sativa Japonica Group] | | | | |
| | | | | &gt:gi\|50878425\|gb\|AAT85199.1\| | | | | |
| | | | | putative ATP synthase beta chain | | | | |
| | | | | [Oryza sativa Japonica Group] | | | | |
| | | | | &gt:gi\|113579812\|dbj\|BAF18175.1\| | | | | |
| | | | | Os05g0553000 [Oryza sativa Japonica Group] | | | | |
| | | | | &gt:gi\|215692634\|dbj\|BAG88054.1\| | | | | |
| | | | | unnamed protein product | | | | |
| | | | | [Oryza sativa Japonica Group] | | | | |
| | | ACS83602 | 242129045 | ATP synthase beta subunit 1 | 0.900362 | Gossypium hirsutum | 3473 | 4801 |
| | | | | [Gossypium hirsutum] | | | | |
| Predicted | 507-526 | EEE57671 | 54398660 | hypothetical protein OsJ_08115 | 1 | Oryza sativa Japonica Group | 3474 | |
| zma | | | | [Oryza sativa Japonica Group] | | | | |
| mir | | | | | | | | |
| 50480 | | | | | | | | |
| | | EAY87257 | 54362548 | hypothetical protein OsI_08658 | 0.984305 | Oryza sativa Indica Group | 3475 | |
| | | | | [Oryza sativa Indica Group] | | | | |
| | | NP_001146323 | 226494858 | hypothetical protein LOC100279899 | 0.826233 | Zea mays | 3476 | 4802 |
| | | | | [Zea mays] | | | | |
| | | | | &gt:gi\|219886633\|gb\|ACL53691.1\| | | | | |
| | | | | unknown [Zea mays] | | | | |
| | | BAJ88744 | 326523406 | predicted protein | 0.817265 | Hordeum vulgare subsp. vulgare | 3477 | 4803 |
| | | | | [Hordeum vulgare subsp. vulgare] | | | | |
| | 543-562 | AAV64247 | 55741096 | unknown [Zea mays] | 1 | Zea mays | 3478 | 4804 |
| | | AAV64209 | 55741054 | unknown [Zea mays] | 0.829971 | Zea mays | 3479 | 4805 |
| | 247-266 | ACG33184 | 195622707 | integral membrane protein DUF6 containing protein | 1 | Zea mays | 3480 | 4806 |
| | | | | [Zea mays] | | | | |
| | | | | &gt:gi\|223948881\|gb\|ACN28524.1\| | | | | |
| | | | | unknown [Zea mays] | | | | |
| | | XP_002462410 | 242049331 | hypothetical protein SORBIDRAFT_02g025210 | 0.879357 | Sorghum bicolor | 3481 | 4807 |
| | | | | [Sorghum bicolor] | | | | |
| | | | | &gt:gi\|241925787\|gb\|EER98931.1\| | | | | |
| | | | | hypothetical protein SORBIDRAFT_02g025210 | | | | |
| | | | | [Sorghum bicolor] | | | | |
| | | BAH01249 | 116011645 | unnamed protein product | 0.729223 | Oryza sativa Japonica Group | 3482 | 4808 |
| | | | | [Oryza sativa Japonica Group] | | | | |
| | | EEC84606 | 54362548 | hypothetical protein OsI_31435 | 0.713137 | Oryza sativa Indica Group | 3483 | |
| | | | | [Oryza sativa Indica Group] | | | | |
| | | | | &gt:gi\|222641609\|gb\|EEE69741.1\| | | | | |
| | | | | hypothetical protein OsJ_29432 | | | | |
| | | | | [Oryza sativa Japonica Group] | | | | |
| | 533-552 | NP_001147862 | 226497613 | LOC100281472 [Zea mays] | 1 | Zea mays | 3484 | 4809 |
| | | | | &gt:gi\|195614188\|gb\|ACG28924.1\| | | | | |
| | | | | transparent testa 12 protein | | | | |
| | | | | [Zea mays] | | | | |
| | 1939-1958 | XP_002463844 | 242032898 | hypothetical protein SORBIDRAFT_01g007310 | 1 | Sorghum bicolor | 3485 | 4810 |
| | | | | [Sorghum bicolor] | | | | |
| | | | | &gt:gi\|242033607\|ref\|XP_002464198.1\| | | | | |
| | | | | hypothetical protein SORBIDRAFT_01g013970 | | | | |
| | | | | [Sorghum bicolor] | | | | |
| | | | | &gt:gi\|241917698\|gb\|EER90842.1\| | | | | |
| | | | | hypothetical protein SORBIDRAFT_01g007310 | | | | |
| | | | | [Sorghum bicolor] | | | | |
| | | | | &gt:gi\|241918052\|gb\|EER91196.1\| | | | | |
| | | | | hypothetical protein SORBIDRAFT_01g013970 | | | | |
| | | | | [Sorghum bicolor] | | | | |
| | | NP_001151463 | 226529446 | LOC100285096 [Zea mays] | 0.941099 | Zea mays | 3486 | 4811 |
| | | | | &gt:gi\|195646966\|gb\|ACG42951.1\| | | | | |
| | | | | subtilisin-like protease precursor | | | | |
| | | | | [Zea mays] | | | | |
| | | NP_001051353 | 115455504 | Os03g0761500 [Oryza sativa Japonica Group] | 0.875654 | Oryza sativa Japonica Group | 3487 | 4812 |
| | | | | &gt:gi\|14488360\|gb\|AAK63927.1\|AC084282_8 | | | | |
| | | | | putative serine protease | | | | |
| | | | | [Oryza sativa Japonica Group] | | | | |
| | | | | &gt:gi\|108711215\|gb\|ABF99010.1\| | | | | |
| | | | | cucumisin-like serine protease, putative, expressed | | | | |
| | | | | [Oryza sativa Japonica Group] | | | | |
| | | | | &gt:gi\|113549824\|dbj\|BAF13267.1\| | | | | |
| | | | | Os03g0761500 [Oryza sativa Japonica Group] | | | | |
| | | | | &gt:gi\|125588004\|gb\|EAZ28668.1\| | | | | |
| | | | | hypothetical protein OsJ_12679 | | | | |
| | | | | [Oryza sativa Japonica Group] | | | | |
| | | | | &gt:gi\|215678732\|dbj\|BAG95169.1\| | | | | |
| | | | | unnamed protein product | | | | |
| | | | | [Oryza sativa Japonica Group] | | | | |
| | | | | &gt:gi\|215737049\|dbj\|BAG95978.1\| | | | | |
| | | | | unnamed protein product | | | | |
| | | | | [Oryza sativa Japonica Group] | | | | |
| | | BAJ99493 | 326508451 | predicted protein | 0.823298 | Hordeum vulgare subsp. vulgare | 3488 | 4813 |
| | | | | [Hordeum vulgare subsp. vulgare] | | | | |
| | 529-548 | ACN27570 | 223946972 | unknown [Zea mays] | 1 | Zea mays | 3489 | 4814 |
| | | EEC76224 | 54362548 | hypothetical protein OsI_13631 | 0.764368 | Oryza sativa Indica Group | 3490 | |
| | | | | [Oryza sativa Indica Group] | | | | |
| | 281-300 | XP_002467528 | 242040266 | hypothetical protein SORBIDRAFT_01g029660 | 1 | Sorghum bicolor | 3491 | 4815 |
| | | | | [Sorghum bicolor] | | | | |
| | | | | &gt:gi\|241921382\|gb\|EER94526.1\| | | | | |
| | | | | hypothetical protein SORBIDRAFT_01g029660 | | | | |
| | | | | [Sorghum bicolor] | | | | |
| | | NP_001147577 | 226531032 | proline oxidase [Zea mays] | 0.893788 | Zea mays | 3492 | 4816 |
| | | | | &gt:gi\|195612286\|gb\|ACG27973.1\| | | | | |
| | | | | proline oxidase [Zea mays] | | | | |
| | | NP_001147660 | 226505515 | proline oxidase [Zea mays] | 0.833667 | Zea mays | 3493 | 4817 |
| | | | | &gt:gi\|195612896\|gb\|ACG28278.1\| | | | | |
| | | | | proline oxidase [Zea mays] | | | | |
| | | BAK02892 | 326505009 | predicted protein | 0.787575 | Hordeum vulgare subsp. vulgare | 3494 | 4818 |
| | | | | [Hordeum vulgare subsp. vulgare] | | | | |
| | | EAY79449 | 54362548 | hypothetical protein OsI_34579 | 0.749499 | Oryza sativa Indica Group | 3495 | |
| | | | | [Oryza sativa Indica Group] | | | | |
| | | NP_001065321 | 115483301 | Os10g0550900 [Oryza sativa Japonica Group] | 0.749499 | Oryza sativa Japonica Group | 3496 | 4819 |
| | | | | &gt:gi\|78708991\|gb\|ABB47966.1\| | | | | |
| | | | | Proline dehydrogenase family protein, expressed | | | | |
| | | | | [Oryza sativa Japonica Group] | | | | |
| | | | | &gt:gi\|113639853\|dbj\|BAF27158.1\| | | | | |
| | | | | Os10g0550900 [Oryza sativa Japonica Group] | | | | |
| | | | | &gt:gi\|215768044\|dbj\|BAH00273.1\| | | | | |
| | | | | unnamed protein product | | | | |
| | | | | [Oryza sativa Japonica Group] | | | | |
| | | AAG13467 | 20143586 | putative proline oxidase | 0.731463 | Oryza sativa Japonica Group | 3497 | 4820 |
| | | | | [Oryza sativa Japonica Group] | | | | |
| | 471-490 | AAV64209 | 55741054 | unknown [Zea mays] | 1 | Zea mays | 3498 | 4821 |
| | 789-808 | NP_001151285 | 226528965 | plastid-specific 30S ribosomal protein 1 [Zea mays] | 1 | Zea mays | 3499 | 4822 |
| | | | | &gt:gi\|195645526\|gb\|ACG42231.1\| plastid-specific | | | | |
| | | | | 30S ribosomal protein 1 [Zea mays] | | | | |
| | | XP_002466065 | 242037340 | hypothetical protein SORBIDRAFT_01g000590 | 0.779935 | Sorghum bicolor | 3500 | 4823 |
| | | | | [Sorghum bicolor] | | | | |
| | | | | &gt:gi\|241919919\|gb\|EER93063.1\| | | | | |
| | | | | hypothetical protein SORBIDRAFT_01g000590 | | | | |
| | | | | [Sorghum bicolor] | | | | |
| | 79-98 | NP_001147623 | 226499377 | WRKY11 - superfamily of TFs having WRKY | 1 | Zea mays | 3501 | 4824 |
| | | | | and zinc finger domains [Zea mays] | | | | |
| | | | | &gt:gi\|194700780\|gb\|ACF84474.1\| | | | | |
| | | | | unknown [Zea mays] | | | | |
| | | | | &gt:gi\|195612626\|gb\|ACG28143.1\| | | | | |
| | | | | WRKY11 - superfamily of TFs having WRKY | | | | |
| | | | | and zinc finger domains [Zea mays] | | | | |
| | | XP_002455987 | 242053682 | hypothetical protein SORBIDRAFT_03g028530 | 0.892045 | Sorghum bicolor | 3502 | 4825 |
| | | | | [Sorghum bicolor] | | | | |
| | | | | &gt:gi\|241927962\|gb\|EES01107.1\| | | | | |
| | | | | hypothetical protein SORBIDRAFT_03g028530 | | | | |
| | | | | [Sorghum bicolor] | | | | |
| | 545-564 | XP_002446670 | 242073467 | hypothetical protein SORBIDRAFT_06g020170 | 1 | Sorghum bicolor | 3503 | 4826 |
| | | | | [Sorghum bicolor] | | | | |
| | | | | &gt:gi\|241937853\|gb\|EES10998.1\| | | | | |
| | | | | hypothetical protein SORBIDRAFT_06g020170 | | | | |
| | | | | [Sorghum bicolor] | | | | |
| | | NP_001151349 | 226528885 | eukaryotic translation initiation factor 4B | 0.905838 | Zea mays | 3504 | 4827 |
| | | | | [Zea mays] | | | | |
| | | | | &gt:gi\|195646008\|gb\|ACG42472.1\| | | | | |
| | | | | eukaryotic translation initiation factor 4B | | | | |
| | | | | [Zea mays] | | | | |
| | | BAH01553 | 116012715 | unnamed protein product | 0.785311 | Oryza sativa Japonica Group | 3505 | 4828 |
| | | | | [Oryza sativa Japonica Group] | | | | |
| | | EEE61194 | 54398660 | hypothetical protein OsJ_15202 | 0.755179 | Oryza sativa Japonica Group | 3506 | |
| | | | | [Oryza sativa Japonica Group] | | | | |
| | | CAE05890 | 38605759 | OSJNBa0044K18.31 | 0.774011 | Oryza sativa Japonica Group | 3507 | 4829 |
| | | | | [Oryza sativa Japonica Group] | | | | |
| | | AAC28254 | 3372235 | eukaryotic translation initiation factor 4B | 0.711864 | Triticum aestivum | 3508 | 4830 |
| | | | | [Triticum aestivum] | | | | |
| | | BAJ97809 | 326531609 | predicted protein | 0.721281 | Hordeum vulgare subsp. vulgare | 3509 | 4831 |
| | | | | [Hordeum vulgare subsp. vulgare] | | | | |
| | 459-478 | NP_001168251 | 293332880 | hypothetical protein LOC100382014 | 1 | Zea mays | 3510 | 4832 |
| | | | | [Zea mays] | | | | |
| | | | | &gt:gi\|223947025\|gb\|ACN27596.1\| | | | | |
| | | | | unknown [Zea mays] | | | | |
| | 741-760 | ACN27595 | 223947022 | unknown [Zea mays] | 1 | Zea mays | 3511 | 4833 |
| | | EAZ17908 | 54398660 | hypothetical protein OsJ_33455 | 0.766633 | Oryza sativa Japonica Group | 3512 | |
| | | | | [Oryza sativa Japonica Group] | | | | |
| | | EAY80442 | 54362548 | hypothetical protein OsI_35624 | 0.765609 | Oryza sativa Indica Group | 3513 | |
| | | | | [Oryza sativa Indica Group] | | | | |
| | | NP_001067561 | 115484834 | Os11g0232100 [Oryza sativa Japonica Group] | 0.765609 | Oryza sativa Japonica Group | 3514 | 4834 |
| | | | | &gt:gi\|62734108\|gb\|AAX96217.1\| | | | | |
| | | | | Leucine Rich Repeat, putative | | | | |
| | | | | [Oryza sativa Japonica Group] | | | | |
| | | | | &gt:gi\|77549387\|gb\|ABA92184.1\| | | | | |
| | | | | Leucine Rich Repeat family protein, expressed | | | | |
| | | | | [Oryza sativa Japonica Group] | | | | |
| | | | | &gt:gi\|113644783\|dbj\|BAF27924.1\| | | | | |
| | | | | Os11g0232100 [Oryza sativa Japonica Group] | | | | |
| | | BAJ87631 | 326511233 | predicted protein | 0.729785 | Hordeum vulgare subsp. vulgare | 3515 | 4835 |
| | | | | [Hordeum vulgare subsp. vulgare] | | | | |
| | | BAJ97282 | 326526530 | predicted protein | 0.728762 | Hordeum vulgare subsp. vulgare | 3516 | 4836 |
| | | | | [Hordeum vulgare subsp. vulgare] | | | | |
| | 1744-1763 | NP_001159284 | 259490169 | hypothetical protein LOC100304374 | 1 | Zea mays | 3517 | 4837 |
| | | | | [Zea mays] | | | | |
| | | | | &gt:gi\|223943193\|gb\|ACN25680.1\| | | | | |
| | | | | unknown [Zea mays] | | | | |
| | | XP_002463079 | 242050669 | hypothetical protein SORBIDRAFT_02g037440 | 0.856041 | Sorghum bicolor | 3518 | 4838 |
| | | | | [Sorghum bicolor] | | | | |
| | | | | &gt:gi\|241926456\|gb\|EER99600.1\| | | | | |
| | | | | hypothetical protein SORBIDRAFT_02g037440 | | | | |
| | | | | [Sorghum bicolor] | | | | |
| | 549-568 | NP_001047898 | 115448236 | Os02g0710900 [Oryza sativa Japonica Group] | 1 | Oryza sativa Japonica Group | 3519 | 4839 |
| | | | | &gt:gi\|41052647\|dbj\|BAD07495.1\| | | | | |
| | | | | putative growth regulator | | | | |
| | | | | [Oryza sativa Japonica Group] | | | | |
| | | | | &gt:gi\|41052863\|dbj\|BAD07777.1\| | | | | |
| | | | | putative growth regulator | | | | |
| | | | | [Oryza sativa Japonica Group] | | | | |
| | | | | &gt:gi\|113537429\|dbj\|BAF09812.1\| | | | | |
| | | | | Os02g0710900 [Oryza sativa Japonica Group] | | | | |
| | 165-184 | XP_002455851 | 242053410 | hypothetical protein SORBIDRAFT_03g026180 | 1 | Sorghum bicolor | 3520 | 4840 |
| | | | | [Sorghum bicolor] | | | | |
| | | | | &gt:gi\|241927826\|gb\|EES00971.1\| | | | | |
| | | | | hypothetical protein SORBIDRAFT_03g026 180 | | | | |
| | | | | [Sorghum bicolor] | | | | |
| | | NP_001130177 | 212275443 | hypothetical protein LOC100191271 | 0.944149 | Zea mays | 3521 | 4841 |
| | | | | [Zea mays] | | | | |
| | | | | &gt:gi\|194688476\|gb\|ACF78322.1\| | | | | |
| | | | | unknown [Zea mays] | | | | |
| | | | | &gt:gi\|195639048\|gb\|ACG38992.1\| | | | | |
| | | | | integral membrane protein [Zea mays] | | | | |
| | | | | &gt:gi\|219888143\|gb\|ACL54446.1\| | | | | |
| | | | | unknown [Zea mays] | | | | |
| | | | | &gt:gi\|224030309\|gb\|ACN34230.1\| | | | | |
| | | | | unknown [Zea mays] | | | | |
| | | NP_001043423 | 115437959 | Os01g0585100 [Oryza sativa Japonica Group] | 0.867021 | Oryza sativa Japonica Group | 3522 | 4842 |
| | | | | &gt:gi\|14588680\|dbj\|BAB61845.1\| | | | | |
| | | | | unknown protein | | | | |
| | | | | [Oryza sativa Japonica Group] | | | | |
| | | | | &gt:gi\|21644683\|dbj\|BAC01240.1\| | | | | |
| | | | | unknown protein | | | | |
| | | | | [Oryza sativa Japonica Group] | | | | |
| | | | | &gt:gi\|113532954\|dbj\|BAF05337.1\| | | | | |
| | | | | Os01g0585100 [Oryza sativa Japonica Group] | | | | |
| | | | | &gt:gi\|215697604\|dbj\|BAG91598.1\| | | | | |
| | | | | unnamed protein product | | | | |
| | | | | [Oryza sativa Japonica Group] | | | | |
| | | EAZ12481 | 54398660 | hypothetical protein OsJ_02378 | 0.867021 | Oryza sativa Japonica Group | 3523 | |
| | | | | [Oryza sativa Japonica Group] | | | | |
| | | EAY74703 | 54362548 | hypothetical protein OsI_02596 | 0.864362 | Oryza sativa Indica Group | 3524 | |
| | | | | [Oryza sativa Indica Group] | | | | |
| | | BAJ96508 | 326517031 | predicted protein | 0.856383 | Hordeum vulgare subsp. vulgare | 3525 | 4843 |
| | | | | [Hordeum vulgare subsp. vulgare] | | | | |
| | | XP_002280579 | 225444823 | PREDICTED: hypothetical protein | 0.712766 | Vitis vinifera | 3526 | 4844 |
| | | | | [Vitis vinifera] | | | | |
| | | ACU22841 | 255644677 | unknown [Glycine max] | 0.712766 | Glycine max | 3527 | 4845 |
| | | XP_002514086 | 255546052 | conserved hypothetical protein | 0.712766 | Ricinus communis | 3528 | 4846 |
| | | | | [Ricinus communis] | | | | |
| | | | | &gt:gi\|223546542\|gb\|EEF48040.1\| | | | | |
| | | | | conserved hypothetical protein | | | | |
| | | | | [Ricinus communis] | | | | |
| | 1215-1234 | NP_001146872 | 226501845 | gibberellin 2-beta-dioxygenase | 1 | Zea mays | 3529 | 4847 |
| | | | | [Zea mays] | | | | |
| | | | | &gt:gi\|195604518\|gb\|ACG24089.1\| | | | | |
| | | | | gibberellin 2-beta-dioxygenase | | | | |
| | | | | [Zea mays] | | | | |
| | | BAJ88432 | 326522772 | predicted protein | 0.742604 | Hordeum vulgare subsp. vulgare | 3530 | 4848 |
| | | | | [Hordeum vulgare subsp. vulgare] | | | | |
| | 725-744 | EEE68876 | 54398660 | hypothetical protein OsJ_27688 | 1 | Oryza sativa Japonica Group | 3531 | |
| | | | | [Oryza sativa Japonica Group] | | | | |
| | 1926-1945 | NP_001159342 | 259489829 | hypothetical protein LOC100304436 | 1 | Zea mays | 3532 | 4849 |
| | | | | [Zea mays] | | | | |
| | | | | &gt:gi\|223943517\|gb\|ACN25842.1\| | | | | |
| | | | | unknown [Zea mays] | | | | |
| | 632-651 | XP_002449308 | 242068062 | hypothetical protein SORBIDRAFT_05g007490 | 1 | Sorghum bicolor | 3533 | 4850 |
| | | | | [Sorghum bicolor] | | | | |
| | | | | &gt:gi\|241935151\|gb\|EES08296.1\| | | | | |
| | | | | hypothetical protein SORBIDRAFT_05g007490 | | | | |
| | | | | [Sorghum bicolor] | | | | |
| | 1268-1287 | XP_002443799 | 242078060 | hypothetical protein SORBIDRAFT_07g002260 | 1 | Sorghum bicolor | 3534 | 4851 |
| | | | | [Sorghum bicolor] | | | | |
| | | | | &gt:gi\|241940149\|gb\|EES13294.1\| | | | | |
| | | | | hypothetical protein SORBIDRAFT_07g002260 | | | | |
| | | | | [Sorghum bicolor] | | | | |
| | | BAJ93280 | 326526206 | predicted protein | 0.767241 | Hordeum vulgare subsp. vulgare | 3535 | 4852 |
| | | | | [Hordeum vulgare subsp. vulgare] | | | | |
| | 189-208 | NP_001151301 | 226508349 | protein binding protein [Zea mays] | 1 | Zea mays | 3536 | 4853 |
| | | | | &gt:gi\|195645662\|gb\|ACG42299.1\| | | | | |
| | | | | protein binding protein [Zea mays] | | | | |
| | | XP_002454380 | 242066181 | hypothetical protein SORBIDRAFT_04g029720 | 0.825 | Sorghum bicolor | 3537 | 4854 |
| | | | | [Sorghum bicolor] | | | | |
| | | | | &gt:gi\|241934211\|gb\|EES07356.1\| | | | | |
| | | | | hypothetical protein SORBIDRAFT_04g029720 | | | | |
| | | | | [Sorghum bicolor] | | | | |
| | | NP_001152525 | 226508021 | protein binding protein [Zea mays] | 0.79 | Zea mays | 3538 | 4855 |
| | | | | &gt:gi\|195657115\|gb\|ACG48025.1\| | | | | |
| | | | | protein binding protein [Zea mays] | | | | |
| | 241-260 | NP_001144625 | 226498917 | hypothetical protein LOC100277643 | 1 | Zea mays | 3539 | 4856 |
| | | | | [Zea mays] | | | | |
| | | | | &gt:gi\|195644788\|gb\|ACG41862.1\| | | | | |
| | | | | hypothetical protein [Zea mays] | | | | |
| | | ACR36806 | 238011541 | unknown [Zea mays] | 0747967 | Zea mays | 3540 | 4857 |
| | 135-154 | NP_001151654 | 226533332 | anthocyanin regulatory C1 protein [Zea mays] | 1 | Zea mays | 3541 | 4858 |
| | | | | &gt:gi\|195648418\|gb\|ACG43677.1lanthocyanin | | | | |
| | | | | regulatory C1 protein [Zea mays] | | | | |
| | 317-336 | NP_001148026 | 226530490 | RING-H2 finger protein ATL2K [Zea mays] | 1 | Zea mays | 3542 | 4859 |
| | | | | &gt:gi\|195615316\|gb\|ACG29488.1\| | | | | |
| | | | | RING-H2 finger protein ATL2K [Zea mays] | | | | |
| | | XP_002456372 | 242054452 | hypothetical protein SORBIDRAFT_03g034930 | 0.863071 | Sorghum bicolor | 3543 | 4860 |
| | | | | [Sorghum bicolor] | | | | |
| | | | | &gt:gi\|241928347\|gb\|EES01492.1\| | | | | |
| | | | | hypothetical protein SORBIDRAFT_03g034930 | | | | |
| | | | | [Sorghum bicolor] | | | | |
| | | NP_001148308 | 226499733 | RING-H2 finger protein ATL2K [Zea mays] | 0.792531 | Zea mays | 3544 | 4861 |
| | | | | &gt:gi\|195617376\|gb\|ACG30518.1\| | | | | |
| | | | | RING-H2 finger protein ATL2K [Zea mays] | | | | |
| | | ACN33508 | 224028864 | unknown [Zea mays] | 0.809129 | Zea mays | 3545 | 4862 |
| Predicted | 22-Mar | EEC68352 | 54362548 | hypothetical protein OsI_36482 | 1 | Oryza sativa Indica Group | 3546 | |
| zma | | | | [Oryza sativa Indica Group] | | | | |
| mir | | | | | | | | |
| 50481 | | | | | | | | |
| | | NP_001068093 | 297612029 | Os11g0557700 [Oryza sativa Japonica Group] | 0.995951 | Oryza sativa Japonica Group | 3547 | 4863 |
| | | | | &gt:gi\|77551490\|gb\|ABA94287.1\| | | | | |
| | | | | protease, putative, expressed | | | | |
| | | | | [Oryza sativa Japonica Group] | | | | |
| | | | | &gt:gi\|113645315\|dbj\|BAF28456.1\| | | | | |
| | | | | Os11g0557700 [Oryza sativa Japonica Group] | | | | |
| | | | | &gt:gi\|215701208\|dbj\|BAG92632.1\| | | | | |
| | | | | unnamed protein product | | | | |
| | | | | [Oryza sativa Japonica Group] | | | | |
| | | | | &gt:gi\|215741610\|dbj\|BAG98105.1\| | | | | |
| | | | | unnamed protein product | | | | |
| | | | | [Oryza sativa Japonica Group] | | | | |
| | | | | &gt:gi\|222616146\|gb\|EEE52278.1\| | | | | |
| | | | | hypothetical protein OsJ_34257 | | | | |
| | | | | [Oryza sativa Japonica Group] | | | | |
| | | BAJ87085 | 326509738 | predicted protein | 0.882591 | Hordeum vulgare subsp. vulgare | 3548 | 4864 |
| | | | | [Hordeum vulgare subsp. vulgare] | | | | |
| | | NP_001151887 | 226509855 | S-adenosylmethionine-dependent | 0.850202 | Zea mays | 3549 | 4865 |
| | | | | methyltransferase/catalytic [Zea mays] | | | | |
| | | | | &gt:gi\|195650607\|gb\|ACG44771.1\| | | | | |
| | | | | S-adenosylmethionine-dependent | | | | |
| | | | | methyltransferase/catalytic [Zea mays] | | | | |
| | | XP_002449700 | 242068846 | hypothetical protein SORBIDRAFT_05g021800 | 0.765182 | Sorghum bicolor | 3550 | 4866 |
| | | | | [Sorghum bicolor] | | | | |
| | | | | &gt:gi\|241935543\|gb\|EES08688.1\| | | | | |
| | | | | hypothetical protein SORBIDRAFT_05g021800 | | | | |
| | | | | [Sorghum bicolor] | | | | |
| | 336-355 | ACN35785 | 224033418 | unknown [Zea mays] | 1 | Zea mays | 3551 | 4867 |
| | | NP_001147826 | 226499285 | aspartic-type endopeptidase/pepsin A | 0.93186 | Zea mays | 3552 | 4868 |
| | | | | [Zea mays] | | | | |
| | | | | &gt:gi\|195613980\|gb\|ACG28820.1\| | | | | |
| | | | | aspartic-type endopeptidase/pepsin A | | | | |
| | | | | [Zea mays] | | | | |
| | | XP_002437603 | 242094225 | hypothetical protein SORBIDRAFT_10g030330 | 0.756906 | Sorghum bicolor | 3553 | 4869 |
| | | | | [Sorghum bicolor] | | | | |
| | | | | &gt:gi\|241915826\|gb\|EER88970.1\| | | | | |
| | | | | hypothetical protein SORBIDRAFT_10g030330 | | | | |
| | | | | [Sorghum bicolor] | | | | |
| | 88-107 | BAK07450 | 326520382 | predicted protein | 1 | Hordeum vulgare subsp. vulgare | 3554 | 4870 |
| | | | | [Hordeum vulgare subsp. vulgare] | | | | |
| | | BAJ96089 | 326512215 | predicted protein | 0.999001 | Hordeum vulgare subsp. vulgare | 3555 | 4871 |
| | | | | [Hordeum vulgare subsp. vulgare] | | | | |
| | | BAJ86216 | 326503419 | predicted protein | 0.953047 | Hordeum vulgare subsp. vulgare | 3556 | 4872 |
| | | | | [Hordeum vulgare subsp. vulgare] | | | | |
| | | NP_001182877 | 308081604 | hypothetical protein LOC100501149 | 0.865135 | Zea mays | 3557 | 4873 |
| | | | | [Zea mays] | | | | |
| | | | | &gt:gi\|238007924\|gb\|ACR34997.1\| | | | | |
| | | | | unknown [Zea mays] | | | | |
| | | BAD25508 | 41152728 | putative copper-exporting ATPase | 0.875125 | Oryza sativa Japonica Group | 3558 | |
| | | | | [Oryza sativa Japonica Group] | | | | |
| | | NP_001046033 | 115444506 | Os02g0172600 [Oryza sativa Japonica Group] | 0.875125 | Oryza sativa Japonica Group | 3559 | 4874 |
| | | | | &gt:gi\|113535564\|dbj\|BAF07947.1\| | | | | |
| | | | | Os02g0172600 [Oryza sativa Japonica Group] | | | | |
| | | EEC72585 | 54362548 | hypothetical protein OsI_06035 | 0.87013 | Oryza sativa Indica Group | 3560 | |
| | | | | [Oryza sativa Indica Group] | | | | |
| | | BAJ96159 | 326514343 | predicted protein | 0.827173 | Hordeum vulgare subsp. vulgare | 3561 | 4875 |
| | | | | [Hordeum vulgare subsp. vulgare] | | | | |
| | | | | &gt:gi\|326528265\|dbj\|BAJ93314.1\| | | | | |
| | | | | predicted protein | | | | |
| | | | | [Hordeum vulgare subsp. vulgare] | | | | |
| | | XP_002438813 | 242096645 | hypothetical protein SORBIDRAFT_10g026600 | 0.822178 | Sorghum bicolor | 3562 | 4876 |
| | | | | [Sorghum bicolor] | | | | |
| | | | | &gt:gi\|241917036\|gb\|EER90180.1\| | | | | |
| | | | | hypothetical protein SORBIDRAFT_10g026600 | | | | |
| | | | | [Sorghum bicolor] | | | | |
| | 666-685 | NP_001150862 | 226510390 | LOC100284495 [Zea mays] | 1 | Zea mays | 3563 | 4877 |
| | | | | &gt:gi\|195642440\|gb\|ACG40688.1\| | | | | |
| | | | | helix-loop-helix DNA-binding domain containing | | | | |
| | | | | protein [Zea mays] | | | | |
| | | XP_002456792 | 242055292 | hypothetical protein SORBIDRAFT_03g042860 | 0.846797 | Sorghum bicolor | 3564 | 4878 |
| | | | | [Sorghum bicolor] | | | | |
| | | | | &gt:gi\|241928767\|gb\|EES01912.1\| | | | | |
| | | | | hypothetical protein SORBIDRAFT_03g042860 | | | | |
| | | | | [Sorghum bicolor] | | | | |
| | 493-512 | NP_001144018 | 226499443 | hypothetical protein LOC100276839 | 1 | Zea mays | 3565 | 4879 |
| | | | | [Zea mays] | | | | |
| | | | | &gt:gi\|195635535\|gb\|ACG37236.1\| | | | | |
| | | | | hypothetical protein [Zea mays] | | | | |
| | | XP_002438996 | 242097011 | hypothetical protein SORBIDRAFT_10g029660 | 0.84058 | Sorghum bicolor | 3566 | 4880 |
| | | | | [Sorghum bicolor] | | | | |
| | | | | &gt:gi\|241917219\|gb\|EER90363.1\| | | | | |
| | | | | hypothetical protein SORBIDRAFT_10g029660 | | | | |
| | | | | [Sorghum bicolor] | | | | |
| | 330-349 | NP_001105960 | 162460273 | WUS1 protein [Zea mays] | 1 | Zea mays | 3567 | 4881 |
| | | | | &gt:gi\|116811056\|emb\|CAJ84136.1\| | | | | |
| | | | | WUS1 protein [Zea mays] | | | | |
| | | XP_002448707 | 242077541 | hypothetical protein SORBIDRAFT_06g031880 | 0.705128 | Sorghum bicolor | 3568 | 4882 |
| | | | | [Sorghum bicolor] | | | | |
| | | | | &gt:gi\|241939890\|gb\|EES13035.1\| | | | | |
| | | | | hypothetical protein SORBIDRAFT_06g03 1880 | | | | |
| | | | | [Sorghum bicolor] | | | | |
| | 739-758 | ACF82946 | 194697723 | unknown [Zea mays] | 1 | Zea mays | 3569 | 4883 |
| | | EAY95236 | 54362548 | hypothetical protein OsI_17053 | 0.761719 | Oryza sativa Indica Group | 3570 | |
| | | | | [Oryza sativa Indica Group] | | | | |
| | | XP_002446966 | 242074059 | hypothetical protein SORBIDRAFT_06g025990 | 0.757813 | Sorghum bicolor | 3571 | 4884 |
| | | | | [Sorghum bicolor] | | | | |
| | | | | &gt:gi\|241938149\|gb\|EES11294.1\| | | | | |
| | | | | hypothetical protein SORBIDRAFT_06g025990 | | | | |
| | | | | [Sorghum bicolor] | | | | |
| | | NP_001053615 | 115460029 | Os04g0573900 [Oryza sativa Japonica Group] | 0.759766 | Oryza sativa Japonica Group | 3572 | 4885 |
| | | | | &gt:gi\|38605952\|emb\|CAD41666.3\| | | | | |
| | | | | OSJNBa0019K04.13 [Oryza sativa Japonica Group] | | | | |
| | | | | &gt:gi\|113565186\|dbj\|BAF15529.1\| | | | | |
| | | | | Os04g0573900 [Oryza sativa Japonica Group] | | | | |
| | | NP_001182854 | 308044212 | hypothetical protein LOC100501109 | 0.728516 | Zea mays | 3573 | 4886 |
| | | | | [Zea mays] | | | | |
| | | | | &gt:gi\|238007736\|gb\|ACR34903.1\| | | | | |
| | | | | unknown [Zea mays] | | | | |
| | 1071-1090 | NP_001145817 | 226491437 | hypothetical protein LOC100279324 | 1 | Zea mays | 3574 | 4887 |
| | | | | [Zea mays] | | | | |
| | | | | &gt:gi\|219884539\|gb\|ACL52644.1\| | | | | |
| | | | | unknown [Zea mays] | | | | |
| | | XP_002460158 | 242044573 | hypothetical protein SORBIDRAFT_02g023600 | 0.773956 | Sorghum bicolor | 3575 | 4888 |
| | | | | [Sorghum bicolor] | | | | |
| | | | | &gt:gi\|241923535\|gb\|EER96679.1\| | | | | |
| | | | | hypothetical protein SORBIDRAFT_02g023600 | | | | |
| | | | | [Sorghum bicolor] | | | | |
| | 153-172 | XP_002455017 | 242051742 | hypothetical protein SORBIDRAFT_03g003020 | 1 | Sorghum bicolor | 3576 | 4889 |
| | | | | [Sorghum bicolor] | | | | |
| | | | | &gt:gi\|241926992\|gb\|EES00137.1\| | | | | |
| | | | | hypothetical protein SORBIDRAFT_03g003020 | | | | |
| | | | | [Sorghum bicolor] | | | | |
| | 513-532 | NP_001140853 | 226509373 | hypothetical protein LOC100272929 | 1 | Zea mays | 3577 | 4890 |
| | | | | [Zea mays] | | | | |
| | | | | &gt:gi\|194701444\|gb\|ACF84806.1\| | | | | |
| | | | | unknown [Zea mays] | | | | |
| | | | | &gt:gi\|194703860\|gb\|ACF86014.1\| | | | | |
| | | | | unknown [Zea mays] | | | | |
| | | | | &gt:gi\|195641672\|gb\|ACG40304.1\| | | | | |
| | | | | hypothetical protein [Zea mays] | | | | |
| | | NP_001144670 | 226506299 | hypothetical protein LOC100277696 | 0.82243 | Zea mays | 3578 | 4891 |
| | | | | [Zea mays] | | | | |
| | | | | &gt:gi\|194708364\|gb\|ACF88266.1\| | | | | |
| | | | | unknown [Zea mays] | | | | |
| | | | | &gt:gi\|195645476\|gb\|ACG42206.1\| | | | | |
| | | | | hypothetical protein [Zea mays] | | | | |
| | | XP_002460312 | 242044881 | hypothetical protein SORBIDRAFT_02g026310 | 0.75 | Sorghum bicolor | 3579 | 4892 |
| | | | | [Sorghum bicolor] | | | | |
| | | | | &gt:gi\|241923689\|gb\|EER96833.1\| | | | | |
| | | | | hypothetical protein SORBIDRAFT_02g026310 | | | | |
| | | | | [Sorghum bicolor] | | | | |
| | 420-439 | NP_001150595 | 226507979 | transferase, transferring glycosyl groups | 1 | Zea mays | 3580 | 4893 |
| | | | | [Zea mays] | | | | |
| | | | | &gt:gi\|195640434\|gb\|ACG39685.1\| | | | | |
| | | | | transferase, transferring glycosyl groups | | | | |
| | | | | [Zea mays] | | | | |
| | 538-557 | EEE57671 | 54398660 | hypothetical protein OsJ_08115 | 1 | Oryza sativa Japonica Group | 3581 | |
| | | | | [Oryza sativa Japonica Group] | | | | |
| | 76-95 | ACN31863 | 223975350 | unknown [Zea mays] | 1 | Zea mays | 3582 | 4894 |
| | | ACG44151 | 195649366 | choline-phosphate cytidylyltransferase B | 0.990164 | Zea mays | 3583 | 4895 |
| | | | | [Zea mays] | | | | |
| | | XP_002445104 | 242080670 | hypothetical protein SORBIDRAFT_07g004150 | 0.95082 | Sorghum bicolor | 3584 | 4896 |
| | | | | [Sorghum bicolor] | | | | |
| | | | | &gt:gi\|241941454\|gb\|EES14599.1\| | | | | |
| | | | | hypothetical protein SORBIDRAFT_07g004150 | | | | |
| | | | | [Sorghum bicolor] | | | | |
| | | NP_001061052 | 115474910 | Os08g0161800 [Oryza sativa Japonica Group] | 0.836066 | Oryza sativa Japonica Group | 3585 | 4897 |
| | | | | &gt:gi\|37806270\|dbj\|BAC99786.1\| | | | | |
| | | | | putative CTP: phosphorylcholine cytidylyltransferase | | | | |
| | | | | [Oryza sativa Japonica Group] | | | | |
| | | | | &gt:gi\|113623021\|dbj\|BAF22966.1\| | | | | |
| | | | | Os08g0161800 [Oryza sativa Japonica Group] | | | | |
| | | | | &gt:gi\|215765435\|dbj\|BAG87132.1\| | | | | |
| | | | | unnamed protein product | | | | |
| | | | | [Oryza sativa Japonica Group] | | | | |
| | | | | &gt:gi\|218200513\|gb\|EEC82940.1\| | | | | |
| | | | | hypothetical protein OsI_27913 | | | | |
| | | | | [Oryza sativa Indica Group] | | | | |
| | | EEE68089 | 54398660 | hypothetical protein OsJ_26135 | 0.832787 | Oryza sativa Japonica Group | 3586 | |
| | | | | [Oryza sativa Japonica Group] | | | | |
| | 32-51 | NP_001137004 | 219362694 | hypothetical protein LOC100217167 | 1 | Zea mays | 3587 | 4898 |
| | | | | [Zea mays] | | | | |
| | | | | &gt:gi\|195639104\|gb\|ACG39020.1\| | | | | |
| | | | | DNA binding protein [Zea mays] | | | | |
| | | | | &gt:gi\|224034497\|gb\|ACN36324.1\| | | | | |
| | | | | unknown [Zea mays] | | | | |
| | | ACF83052 | 194697935 | unknown [Zea mays] | 0.994334 | Zea mays | 3588 | 4899 |
| | | XP_002448987 | 242067420 | hypothetical protein SORBIDRAFT_05g002940 | 0.869688 | Sorghum bicolor | 3589 | 4900 |
| | | | | [Sorghum bicolor] | | | | |
| | | | | &gt:gi\|241934830\|gb\|EES07975.1\| | | | | |
| | | | | hypothetical protein SORBIDRAFT_05g002940 | | | | |
| | | | | [Sorghum bicolor] | | | | |
| | | NP_001065753 | 115484182 | Os11g0149100 [Oryza sativa Japonica Group] | 0.705382 | Oryza sativa Japonica Group | 3590 | 4901 |
| | | | | &gt:gi\|62701672\|gb\|AAX92745.1\| | | | | |
| | | | | expressed protein | | | | |
| | | | | [Oryza sativa Japonica Group] | | | | |
| | | | | &gt:gi\|77548692\|gb\|ABA91489.1\| | | | | |
| | | | | expressed protein | | | | |
| | | | | [Oryza sativa Japonica Group] | | | | |
| | | | | &gt:gi\|113644457\|dbj\|BAF27598.1\| | | | | |
| | | | | Os11g0149100 [Oryza sativa Japonica Group] | | | | |
| | | XP_002441824 | 242082797 | hypothetical protein SORBIDRAFT_08g002940 | 0.798867 | Sorghum bicolor | 3591 | 4902 |
| | | | | [Sorghum bicolor] | | | | |
| | | | | &gt:gi\|241942517\|gb\|EES15662.1\| | | | | |
| | | | | hypothetical protein SORBIDRAFT_08g002940 | | | | |
| | | | | [Sorghum bicolor] | | | | |
| | 573-592 | ACG34322 | 195624983 | fructokinase-2 [Zea mays] | 1 | Zea mays | 3592 | 4903 |
| | | | | &gt:gi\|195626972\|gb\|ACG35316.1\| | | | | |
| | | | | fructokinase-2 [Zea mays] | | | | |
| | | ACG38926 | 195638915 | fructokinase-2 [Zea mays] | 0.997015 | Zea mays | 3593 | 4904 |
| | | ACG34312 | 195624963 | fructokinase-2 [Zea mays] | 0.991045 | Zea mays | 3594 | 4905 |
| | | ACG33449 | 195623237 | fructokinase-2 [Zea mays] | 0.991045 | Zea mays | 3595 | 4906 |
| | | ACG39031 | 195639125 | fructokinase-2 [Zea mays] | 0.98806 | Zea mays | 3596 | 4907 |
| | | NP_001060837 | 115474480 | Os08g0113100 [Oryza sativa Japonica Group] | 0.901493 | Oryza sativa Japonica Group | 3597 | 4908 |
| | | | | &gt:gi\|122234591\|sp\|Q0J8G4.1\|SCRK2_ORYSJ | | | | |
| | | | | RecName: Full = Fructokinase-2; | | | | |
| | | | | AltName: Full = Fructokinase II; | | | | |
| | | | | AltName: Full = OsFKII | | | | |
| | | | | &gt:gi\|158513662\|sp\|A2YQL4.2\|SCRK2_ORYSI | | | | |
| | | | | RecName: Full = Fructokinase-2; | | | | |
| | | | | AltName: Full = Fructokinase II; | | | | |
| | | | | AltName: Full = OsFKII | | | | |
| | | | | &gt:gi\|16566704\|gb\|AAL26573.1\|AF429947_1 | | | | |
| | | | | putative fructokinase II | | | | |
| | | | | [Oryza sativa] | | | | |
| | | | | &gt:gi\|32352126\|dbj\|BAC78556.1\| | | | | |
| | | | | fructokinase [Oryza sativa Japonica Group] | | | | |
| | | | | &gt:gi\|42408363\|dbj\|BAD09515.1\| | | | | |
| | | | | putative fructokinase | | | | |
| | | | | [Oryza sativa Japonica Group] | | | | |
| | | | | &gt:gi\|113622806\|dbj\|BAF22751.1\| | | | | |
| | | | | Os08g0113100 [Oryza sativa Japonica Group] | | | | |
| | | | | &gt:gi\|125601970\|gb\|EAZ41295.1\| | | | | |
| | | | | hypothetical protein OsJ_25803 | | | | |
| | | | | [Oryza sativa Japonica Group] | | | | |
| | | | | &gt:gi\|215687214\|dbj\|BAG91779.1\| | | | | |
| | | | | unnamed protein product | | | | |
| | | | | [Oryza sativa Japonica Group] | | | | |
| | | | | &gt:gi\|215708813\|dbj\|BAG94082.1\| | | | | |
| | | | | unnamed protein product | | | | |
| | | | | [Oryza sativa Japonica Group] | | | | |
| | | EAZ05375 | 54362548 | hypothetical protein OsI_27579 | 0.901493 | Oryza sativa Indica Group | 3598 | |
| | | | | [Oryza sativa Indica Group] | | | | |
| | | BAK06949 | 326513417 | predicted protein | 0.862687 | Hordeum vulgare subsp. vulgare | 3599 | 4909 |
| | | | | [Hordeum vulgare subsp. vulgare] | | | | |
| | 333-352 | XP_002467133 | 242039476 | hypothetical protein SORBIDRAFT_01g020150 | 1 | Sorghum bicolor | 3600 | 4910 |
| | | | | [Sorghum bicolor] | | | | |
| | | | | &gt:gi\|241920987\|gb\|EER94131.1\| | | | | |
| | | | | hypothetical protein SORBIDRAFT_01g020150 | | | | |
| | | | | [Sorghum bicolor] | | | | |
| | | ACN29136 | 223950104 | unknown [Zea mays] | 0.958874 | Zea mays | 3601 | 4911 |
| | | NP_001064764 | 115482341 | Os10g0457600 [Oryza sativa Japonica Group] | 0.904762 | Oryza sativa Japonica Group | 3602 | 4912 |
| | | | | &gt:gi\|14140293\|gb\|AAK54299.1\|AC034258_17 | | | | |
| | | | | putative thiolase [Oryza sativa Japonica Group] | | | | |
| | | | | &gt:gi\|31432470\|gb\|AAP54100.1\| | | | | |
| | | | | 3-ketoacyl-CoA thiolase 2, peroxisomal | | | | |
| | | | | precursor, putative, expressed | | | | |
| | | | | [Oryza sativa Japonica Group] | | | | |
| | | | | &gt:gi\|113639373\|dbj\|BAF26678.1\| | | | | |
| | | | | Os10g0457600 [Oryza sativa Japonica Group] | | | | |
| | | | | &gt:gi\|125575033\|gb\|EAZ16317.1\| | | | | |
| | | | | hypothetical protein OsJ_31778 | | | | |
| | | | | [Oryza sativa Japonica Group] | | | | |
| | | | | &gt:gi\|215704141\|dbj\|BAG92981.1\| | | | | |
| | | | | unnamed protein product | | | | |
| | | | | [Oryza sativa Japonica Group] | | | | |
| | | EEC67095 | 54362548 | hypothetical protein OsI_33888 | 0.904762 | Oryza sativa Indica Group | 3603 | |
| | | | | [Oryza sativa Indica Group] | | | | |
| | | XP_002285653 | 225433423 | PREDICTED: hypothetical protein isoform 1 | 0.772727 | Vitis vinifera | 3604 | 4913 |
| | | | | [Vitis vinifera] | | | | |
| | | | | &gt:gi\|297741919\|emb\|CBI33354.3\| | | | | |
| | | | | unnamed protein product | | | | |
| | | | | [Vitis vinifera] | | | | |
| | | CAN81585 | 147866528 | hypothetical protein VITISV_023191 | 0.772727 | Vitis vinifera | 3605 | |
| | | | | [Vitis vinifera] | | | | |
| | | NP_001131193 | 212723031 | hypothetical protein LOC100192501 | 0.766234 | Zea mays | 3606 | 4914 |
| | | | | [Zea mays] | | | | |
| | | | | &gt:gi\|194690834\|gb\|ACF79501.1\| | | | | |
| | | | | unknown [Zea mays] | | | | |
| | | ACG36949 | 195634960 | 3-ketoacyl-CoA thiolase 2, peroxisomal | 0.764069 | Zea mays | 3607 | 4915 |
| | | | | precursor [Zea mays] | | | | |
| | | XP_002299284 | 224057613 | predicted protein [Populus trichocarpa] | 0.768398 | Populus trichocarpa | 3608 | 4916 |
| | | | | &gt:gi\|222846542\|gb\|EEE84089.1\| | | | | |
| | | | | predicted protein [Populus trichocarpa] | | | | |
| | | ACV70033 | 257815408 | 3-ketoacyl CoA thiolase 2 | 0.764069 | Petunia × hybrida | 3609 | 4917 |
| | | | | [Petunia × hybrida] | | | | |
| | 135-154 | XP_002437297 | 242093613 | hypothetical protein SORBIDRAFT_10g024430 | 1 | Sorghum bicolor | 3610 | 4918 |
| | | | | [Sorghum bicolor] | | | | |
| | | | | &gt:gi\|241915520\|gb\|EER88664.1\| | | | | |
| | | | | hypothetical protein SORBIDRAFT_10g024430 | | | | |
| | | | | [Sorghum bicolor] | | | | |
| | | ACR36817 | 238011563 | unknown [Zea mays] | 0.875 | Zea mays | 3611 | 4919 |
| | | NP_001147256 | 226507503 | LOC100280864 [Zea mays] | 0.871711 | Zea mays | 3612 | 4920 |
| | | | | &gt:gi\|195609146\|gb\|ACG26403.1\| | | | | |
| | | | | bZIP transcription factor protein | | | | |
| | | | | [Zea mays] | | | | |
| | 348-367 | XP_002437473 | 242093965 | hypothetical protein SORBIDRAFT_10g027790 | 1 | Sorghum bicolor | 3613 | 4921 |
| | | | | [Sorghum bicolor] | | | | |
| | | | | &gt:gi\|241915696\|gb\|EER88840.1\| | | | | |
| | | | | hypothetical protein SORBIDRAFT_10g027790 | | | | |
| | | | | [Sorghum bicolor] | | | | |
| | | NP_001058399 | 115469599 | Os06g0685700 [Oryza sativa Japonica Group] | 0.858956 | Oryza sativa Japonica Group | 3614 | 4922 |
| | | | | &gt:gi\|75253259\|sp\|Q653H7.1\|ARFR_ORYSJ | | | | |
| | | | | RecName: Full = Auxin response factor 18; | | | | |
| | | | | AltName: Full = OsARF10 | | | | |
| | | | | &gt:gi\|52076670\|dbj\|BAD45570.1\| | | | | |
| | | | | putative auxin response factor 10 | | | | |
| | | | | [Oryza sativa Japonica Group] | | | | |
| | | | | &gt:gi\|52077007\|dbj\|BAD46040.1\| | | | | |
| | | | | putative auxin response factor 10 | | | | |
| | | | | [Oryza sativa Japonica Group] | | | | |
| | | | | &gt:gi\|113596439\|dbj\|BAF20313.1\| | | | | |
| | | | | Os06g0685700 [Oryza sativa Japonica Group] | | | | |
| | | | | &gt:gi\|215713413\|dbj\|BAG94550.1\| | | | | |
| | | | | unnamed protein product | | | | |
| | | | | [Oryza sativa Japonica Group] | | | | |
| | | BAB85919 | 19352050 | auxin response factor 10 | 0.857546 | Oryza sativa | 3615 | 4923 |
| | | | | [Oryza sativa] | | | | |
| | | EEC81202 | 54362548 | hypothetical protein OsI_24228 | 0.722144 | Oryza sativa Indica Group | 3616 | |
| | | | | [Oryza sativa Indica Group] | | | | |
| | | EEE66240 | 54398660 | hypothetical protein OsJ_22412 | 0.77292 | Oryza sativa Japonica Group | 3617 | |
| | | | | [Oryza sativa Japonica Group] | | | | |
| | 85-104 | BAD12057 | 44885915 | plastidic glutamine synthetase | 1 | Phragmites australis | 3618 | 4924 |
| | | | | [Phragmites australis] | | | | |
| | | | | &gt:gi\|44885918\|dbj\|BAD12058.1\| | | | | |
| | | | | plastidic glutamine synthetase | | | | |
| | | | | [Phragmites australis] | | | | |
| | | NP_001054133 | 115461065 | Os04g0659100 [Oryza sativa Japonica Group] | 0.93007 | Oryza sativa Japonica Group | 3619 | 4925 |
| | | | | &gt:gi\|121343\|sp\|P14655.1\|GLNA2_ORYSJ | | | | |
| | | | | RecName: Full = Glutamine synthetase, chloroplastic; | | | | |
| | | | | AltName: Full = Glutamate--ammonia ligase; | | | | |
| | | | | AltName: Full = OsGS2; | | | | |
| | | | | Short = GS2; Flags: Precursor | | | | |
| | | | | &gt:gi\|20370\|emb\|CAA32462.1\| | | | | |
| | | | | unnamed protein product [Oryza sativa] | | | | |
| | | | | &gt:gi\|38345192\|emb\|CAE02885.2\| | | | | |
| | | | | OSJNBa0015K02.2 [Oryza sativa Japonica Group] | | | | |
| | | | | &gt:gi\|38346409\|emb\|CAE54574.1\| | | | | |
| | | | | OSJNBa0011F23.5 [Oryza sativa Japonica Group] | | | | |
| | | | | &gt:gi\|113565704\|dbj\|BAF16047.1\| | | | | |
| | | | | Os04g0659100 [Oryza sativa Japonica Group] | | | | |
| | | | | &gt:gi\|116310855\|emb\|CAH67797.1\| | | | | |
| | | | | OSIGBa0132E09-OSIGBa0108L24.11 | | | | |
| | | | | [Oryza sativa Indica Group] | | | | |
| | | | | &gt:gi\|218195744\|gb\|EEC78171.1\| | | | | |
| | | | | hypothetical protein OsI_17756 | | | | |
| | | | | [Oryza sativa Indica Group] | | | | |
| | | | | &gt:gi\|222629702\|gb\|EEE61834.1\| | | | | |
| | | | | hypothetical protein OsJ_16481 | | | | |
| | | | | [Oryza sativa Japonica Group] | | | | |
| | | BAD12059 | 44885919 | plastidic glutamine synthetase | 0.913753 | Phragmites australis | 3620 | 4926 |
| | | | | [Phragmites australis] | | | | |
| | | AAZ30062 | 71362639 | plastid glutamine synthetase isoform GS2c | 0.864802 | Triticum aestivum | 3621 | 4927 |
| | | | | [Triticum aestivum] | | | | |
| | | | | &gt:gi\|73672739\|gb\|AAZ80474.1\| GS2 | | | | |
| | | | | [Triticum aestivum] | | | | |
| | | | | &gt:gi\|251832981\|gb\|ACT22493.1\| | | | | |
| | | | | plastid glutamine synthetase 2 | | | | |
| | | | | [Triticum aestivum] | | | | |
| | | | | &gt:gi\|251832984\|gb\|ACT22495.1\| | | | | |
| | | | | plastid glutamine synthetase 2 | | | | |
| | | | | [Triticum aestivum] | | | | |
| | | | | &gt:gi\|251832990\|gb\|ACT22498.1\| | | | | |
| | | | | plastid glutamine synthetase 2 | | | | |
| | | | | [Triticum aestivum] | | | | |
| | | | | &gt:gi\|334855519\|gb\|AEH16638.1\| | | | | |
| | | | | glutamine synthetase | | | | |
| | | | | [Triticum aestivum] | | | | |
| | | BAJ91545 | 326509256 | predicted protein | 0.864802 | Hordeum vulgare subsp. vulgare | 3622 | 4928 |
| | | | | [Hordeum vulgare subsp. vulgare] | | | | |
| | | ACT22496 | 251832985 | plastid glutamine synthetase 2 | 0.862471 | Triticum aestivum | 3623 | 4929 |
| | | | | [Triticum aestivum] | | | | |
| | | | | &gt:gi\|251832988\|gb\|ACT22497.1\| | | | | |
| | | | | plastid glutamine synthetase 2 | | | | |
| | | | | [Triticum aestivum] | | | | |
| | | ACT22500 | 251832991 | plastid glutamine synthetase 2 | 0.862471 | Triticum aestivum | 3624 | 4930 |
| | | | | [Triticum aestivum] | | | | |
| | | CAA34131 | 18985 | unnamed protein product | 0.862471 | Hordeum vulgare subsp. vulgare | 3625 | 4931 |
| | | | | [Hordeum vulgare subsp. vulgare] | | | | |
| | | BAJ95492 | 326505641 | predicted protein | 0.86014 | Hordeum vulgare subsp. vulgare | 3626 | 4932 |
| | | | | [Hordeum vulgare subsp. vulgare] | | | | |
| | | AAL87183 | 19387261 | putative precursor chloroplastic glutamine | 0.902098 | Oryza sativa Japonica Group | 3627 | |
| | | | | synthetase [Oryza sativa Japonica Group] | | | | |
| | 252-271 | NP_001182863 | 308080633 | hypothetical protein LOC100501125 | 1 | Zea mays | 3628 | 4933 |
| | | | | [Zea mays] | | | | |
| | | | | &gt:gi\|238007838\|gb\|ACR34954.1\| | | | | |
| | | | | unknown [Zea mays] | | | | |
| | | AAK28346 | 13506811 | receptor-like protein kinase 1 [Zea mays] | 0.871329 | Zea mays | 3629 | 4934 |
| | | XP_002445874 | 242082210 | hypothetical protein SORBIDRAFT_07g027220 | 0.815385 | Sorghum bicolor | 3630 | 4935 |
| | | | | [Sorghum bicolor] | | | | |
| | | | | &gt:gi\|241942224\|gb\|EES15369.1\| | | | | |
| | | | | hypothetical protein SORBIDRAFT_07g027220 | | | | |
| | | | | [Sorghum bicolor] | | | | |
| | 888-907 | ACG36535 | 195629787 | fructokinase-2 [Zea mays] | 1 | Zea mays | 3631 | 4936 |
| | | NP_001060837 | 115474480 | Os08g0113100 [Oryza sativa Japonica Group] | 0.901493 | Oryza sativa Japonica Group | 3632 | 4937 |
| | | | | &gt:gi\|122234591\|sp\|QOJ8G4.1\|SCRK2_ORYSJ | | | | |
| | | | | RecName: Full = Fructokinase-2; | | | | |
| | | | | AltName: Full = Fructokinase II; | | | | |
| | | | | AltName: Full = OsFKII | | | | |
| | | | | &gt:gi\|158513662\|sp\|A2YQL4.2\|SCRK2_ORYSI | | | | |
| | | | | RecName: Full = Fructokinase-2; | | | | |
| | | | | AltName: Full = Fructokinase II; | | | | |
| | | | | AltName: Full = OsFKII | | | | |
| | | | | &gt:gi\|16566704\|gb\|AAL26573.1\|AF429947_1 | | | | |
| | | | | putative fructokinase II [Oryza sativa] | | | | |
| | | | | &gt:gi\|32352126\|dbj\|BAC78556.1\| | | | | |
| | | | | fructokinase [Oryza sativa Japonica Group] | | | | |
| | | | | &gt:gi\|42408363\|dbj\|BAD09515.1\| | | | | |
| | | | | putative fructokinase | | | | |
| | | | | [Oryza sativa Japonica Group] | | | | |
| | | | | &gt:gi\|113622806\|dbj\|BAF22751.1\| | | | | |
| | | | | Os08g0113100 [Oryza sativa Japonica Group] | | | | |
| | | | | &gt:gi\|125601970\|gb\|EAZ41295.1\| | | | | |
| | | | | hypothetical protein OsJ_25803 | | | | |
| | | | | [Oryza sativa Japonica Group] | | | | |
| | | | | &gt:gi\|215687214\|dbj\|BAG91779.1\| | | | | |
| | | | | unnamed protein product | | | | |
| | | | | [Oryza sativa Japonica Group] | | | | |
| | | | | &gt:gi\|215708813\|dbj\|BAG94082.1\| | | | | |
| | | | | unnamed protein product | | | | |
| | | | | [Oryza sativa Japonica Group] | | | | |
| | 140-159 | ACG30481 | 195617301 | hypothetical protein [Zea mays] | 1 | Zea mays | 3633 | 4938 |
| | | ACG37907 | 195636877 | hypothetical protein [Zea mays] | 0.931034 | Zea mays | 3634 | 4939 |
| | | ACF79048 | 194689927 | unknown [Zea mays] | 0.977011 | Zea mays | 3635 | 4940 |
| | | ACG29425 | 195615189 | hypothetical protein [Zea mays] | 0.95977 | Zea mays | 3636 | 4941 |
| | | | | &gt:gi\|195649199\|gb\|ACG44067.1\| | | | | |
| | | | | hypothetical protein [Zea mays] | | | | |
| | | NP_001144199 | 226501971 | hypothetical protein LOC100277059 | 0.890805 | Zea mays | 3637 | 4942 |
| | | | | [Zea mays] | | | | |
| | | | | &gt:gi\|195638304\|gb\|ACG38620.1\| | | | | |
| | | | | hypothetical protein [Zea mays] | | | | |
| | | EEC79730 | 54362548 | hypothetical protein OsI_21063 | 0.787356 | Oryza sativa Indica Group | 3638 | |
| | | | | [Oryza sativa Indica Group] | | | | |
| | | XP_002441542 | 242091418 | hypothetical protein SORBIDRAFT_09g029060 | 0.827586 | Sorghum bicolor | 3639 | 4943 |
| | | | | [Sorghum bicolor] | | | | |
| | | | | &gt:gi\|241946827\|gb\|EES19972.1\| | | | | |
| | | | | hypothetical protein SORBIDRAFT_09g029060 | | | | |
| | | | | [Sorghum bicolor] | | | | |
| | | NP_001056374 | 115465548 | Os05g0571400 [Oryza sativa Japonica Group] | 0.747126 | Oryza sativa Japonica Group | 3640 | 4944 |
| | | | | &gt:gi\|52353529\|gb\|AAU44095.1\| | | | | |
| | | | | unknown protein | | | | |
| | | | | [Oryza sativa Japonica Group] | | | | |
| | | | | &gt:gi\|113579925\|dbj\|BAF18288.1\| | | | | |
| | | | | Os05g0571400 [Oryza sativa Japonica Group] | | | | |
| | | | | &gt:gi\|215692864\|dbj\|BAG88284.1\| | | | | |
| | | | | unnamed protein product | | | | |
| | | | | [Oryza sativa Japonica Group] | | | | |
| | | BAK01837 | 326489712 | predicted protein | 0.729885 | Hordeum vulgare subsp. vulgare | 3641 | 4945 |
| | | | | [Hordeum vulgare subsp. vulgare] | | | | |
| | | | | &gt:gi\|326513576\|dbj\|BAJ87807.1\| | | | | |
| | | | | predicted protein | | | | |
| | | | | [Hordeum vulgare subsp. vulgare] | | | | |
| | 154-173 | ACN35719 | 224033286 | unknown [Zea mays] | 1 | Zea mays | 3642 | 4946 |
| | 1182-1201 | XP_002445912 | 242082286 | hypothetical protein SORBIDRAFT_07g027930 | 1 | Sorghum bicolor | 3643 | 4947 |
| | | | | [Sorghum bicolor] | | | | |
| | | | | &gt:gi\|241942262\|gb\|EES15407.1\| | | | | |
| | | | | hypothetical protein SORBIDRAFT_07g027930 | | | | |
| | | | | [Sorghum bicolor] | | | | |
| | | NP_001148257 | 226530356 | protein kinase [Zea mays] | 0.853273 | Zea mays | 3644 | 4948 |
| | | | | &gt:gi\|195616992\|gb\|ACG30326.1\| | | | | |
| | | | | protein kinase [Zea mays] | | | | |
| | | | | &gt:gi\|223974019\|gb\|ACN31197.1\| | | | | |
| | | | | unknown [Zea mays] | | | | |
| | 1224-1243 | NP_001130351 | 212274554 | hypothetical protein LOC100191446 | 1 | Zea mays | 3645 | 4949 |
| | | | | [Zea mays] | | | | |
| | | | | &gt:gi\|194688912\|gb\|ACF78540.1\| | | | | |
| | | | | unknown [Zea mays] | | | | |
| | | NP_001168165 | 293331166 | hypothetical protein LOC100381917 | 0.883562 | Zea mays | 3646 | 4950 |
| | | | | [Zea mays] | | | | |
| | | | | &gt:gi\|223946413\|gb\|ACN27290.1\| | | | | |
| | | | | unknown [Zea mays] | | | | |
| | | XP_002460448 | 242045153 | hypothetical protein SORBIDRAFT_02g028250 | 0.849315 | Sorghum bicolor | 3647 | 4951 |
| | | | | [Sorghum bicolor] | | | | |
| | | | | &gt:gi\|241923825\|gb\|EER96969.1\| | | | | |
| | | | | hypothetical protein SORBIDRAFT_02g028250 | | | | |
| | | | | [Sorghum bicolor] | | | | |
| | | BAJ96874 | 326521341 | predicted protein | 0.719178 | Hordeum vulgare subsp. vulgare | 3648 | 4952 |
| | | | | [Hordeum vulgare subsp. vulgare] | | | | |
| | 621-640 | NP_001132310 | 212723631 | hypothetical protein LOC100193752 | 1 | Zea mays | 3649 | 4953 |
| | | | | [Zea mays] | | | | |
| | | | | &gt:gi\|194694046\|gb\|ACF81107.1\| | | | | |
| | | | | unknown [Zea mays] | | | | |
| | | NP_001150692 | 226531587 | transmembrane receptor [Zea mays] | 0.861842 | Zea mays | 3650 | 4954 |
| | | | | &gt:gi\|195641100\|gb\|ACG40018.1\| | | | | |
| | | | | transmembrane receptor [Zea mays] | | | | |
| | | NP_001183230 | 308079992 | hypothetical protein LOC100501618 | 0.785088 | Zea mays | 3651 | 4955 |
| | | | | [Zea mays] | | | | |
| | | | | &gt:gi\|238010202\|gb\|ACR36136.1\| | | | | |
| | | | | unknown [Zea mays] | | | | |
| | | XP_002439928 | 242088190 | hypothetical protein SORBIDRAFT_09g022750 | 0.791667 | Sorghum bicolor | 3652 | 4956 |
| | | | | [Sorghum bicolor] | | | | |
| | | | | &gt:gi\|241945213\|gb\|EES18358.1\| | | | | |
| | | | | hypothetical protein SORBIDRAFT_09g022750 | | | | |
| | | | | [Sorghum bicolor] | | | | |
| | 315-334 | NP_001147345 | 226510092 | AP2 domain containing protein [Zea mays] | 1 | Zea mays | 3653 | 4957 |
| | | | | &gt:gi\|195610330\|gb\|ACG26995.1\| | | | | |
| | | | | AP2 domain containing protein [Zea mays] | | | | |
| | | XP_002452378 | 242062177 | hypothetical protein SORBIDRAFT_04g024690 | 0.884354 | Sorghum bicolor | 3654 | 4958 |
| | | | | [Sorghum bicolor] | | | | |
| | | | | &gt:gi\|241932209\|gb\|EES05354.1\| | | | | |
| | | | | hypothetical protein SORBIDRAFT_04g024690 | | | | |
| | | | | [Sorghum bicolor] | | | | |
| | 1284-1303 | XP_002453161 | 242063743 | hypothetical protein SORBIDRAFT_04g001020 | 1 | Sorghum bicolor | 3655 | 4959 |
| | | | | [Sorghum bicolor] | | | | |
| | | | | &gt:gi\|241932992\|gb\|EES06137.1\| | | | | |
| | | | | hypothetical protein SORBIDRAFT_04g001020 | | | | |
| | | | | [Sorghum bicolor] | | | | |
| | | NP_001132593 | 212722739 | hypothetical protein LOC100194065 | 0.852332 | Zea mays | 3656 | 4960 |
| | | | | [Zea mays] | | | | |
| | | | | &gt:gi\|194694848\|gb\|ACF81508.1\| | | | | |
| | | | | unknown [Zea mays] | | | | |
| | | XP_002445672 | 242081806 | hypothetical protein SORBIDRAFT_07g023970 | 0.725389 | Sorghum bicolor | 3657 | 4961 |
| | | | | [Sorghum bicolor] | | | | |
| | | | | &gt:gi\|241942022\|gb\|EES15167.1\| | | | | |
| | | | | hypothetical protein SORBIDRAFT_07g023970 | | | | |
| | | | | [Sorghum bicolor] | | | | |
| | | EAZ13316 | 54398660 | hypothetical protein OsJ_03238 | 0.722798 | Oryza sativa Japonica Group | 3658 | |
| | | | | [Oryza sativa Japonica Group] | | | | |
| | | BAD13218 | 42409221 | putative MtN21 | 0.722798 | Oryza sativa Japonica Group | 3659 | 4962 |
| | | | | [Oryza sativa Japonica Group] | | | | |
| | | NP_001172775 | 297720824 | Os02g0114050 [Oryza sativa Japonica Group] | 0.733161 | Oryza sativa Japonica Group | 3660 | 4963 |
| | | | | &gt:gi\|41052583\|dbj\|BAD07925.1\| | | | | |
| | | | | putative nodulin MIN21 | | | | |
| | | | | [Oryza sativa Japonica Group] | | | | |
| | | | | &gt:gi\|41052778\|dbj\|BAD07647.1\| | | | | |
| | | | | putative nodulin MIN21 | | | | |
| | | | | [Oryza sativa Japonica Group] | | | | |
| | | | | &gt:gi\|125580551\|gb\|EAZ21482.1\| | | | | |
| | | | | hypothetical protein OsJ_05091 | | | | |
| | | | | [Oryza sativa Japonica Group] | | | | |
| | | | | &gt:gi\|218189915\|gb\|EEC72342.1\| | | | | |
| | | | | hypothetical protein OsI_05563 | | | | |
| | | | | [Oryza sativa Indica Group] | | | | |
| | | | | &gt:gi\|255670546\|dbj\|BAH91504.1\| | | | | |
| | | | | Os02g0114050 [Oryza sativa Japonica Group] | | | | |
| | | EAZ08018 | 54362548 | hypothetical protein OsI_30283 | 0.720207 | Oryza sativa Indica Group | 3661 | |
| | | | | [Oryza sativa Indica Group] | | | | |
| | 760-779 | XP_002446967 | 242074061 | hypothetical protein SORBIDRAFT_06g026000 | 1 | Sorghum bicolor | 3662 | 4964 |
| | | | | [Sorghum bicolor] | | | | |
| | | | | &gt:gi\|241938150\|gb\|EES11295.1\| | | | | |
| | | | | hypothetical protein SORBIDRAFT_06g026000 | | | | |
| | | | | [Sorghum bicolor] | | | | |
| | 621-640 | NP_001104903 | 162460262 | protein terminal ear1 [Zea mays] | 1 | Zea mays | 3663 | 4965 |
| | | | | &gt:gi\|75318510\|sp\|O65001.1\|TE1_MAIZE | | | | |
| | | | | RecName: Full = Protein terminal ear1 | | | | |
| | | | | &gt:gi\|13540340\|gb\|AAK29419.1\|AF348319_1 | | | | |
| | | | | TERMINAL EAR1 [Zea mays] | | | | |
| | | | | &gt:gi\|3153237\|gb\|AAC39463.1\| | | | | |
| | | | | terminal ear1 [Zea mays] | | | | |
| | | XP_002456810 | 242055328 | hypothetical protein SORBIDRAFT_03g043230 | 0.858232 | Sorghum bicolor | 3664 | 4966 |
| | | | | [Sorghum bicolor] | | | | |
| | | | | &gt:gi\|241928785\|gb\|EES01930.1\| | | | | |
| | | | | hypothetical protein SORBIDRAFT_03g043230 | | | | |
| | | | | [Sorghum bicolor] | | | | |
| | 84-103 | XP_002437444 | 242093907 | hypothetical protein SORBIDRAFT_10g027180 | 1 | Sorghum bicolor | 3665 | 4967 |
| | | | | [Sorghum bicolor] | | | | |
| | | | | &gt:gi\|241915667\|gb\|EER88811.1\| | | | | |
| | | | | hypothetical protein SORBIDRAFT_10g027180 | | | | |
| | | | | [Sorghum bicolor] | | | | |
| | | NP_001151503 | 226492640 | rab geranylgeranyl transferase like protein | 0.935065 | Zea mays | 3666 | 4968 |
| | | | | [Zea mays] | | | | |
| | | | | &gt:gi\|195647272\|gb\|ACG43104.1\| | | | | |
| | | | | rab geranylgeranyl transferase like protein | | | | |
| | | | | [Zea mays] | | | | |
| | | BAJ96308 | 326514641 | predicted protein | 0.767677 | Hordeum vulgare subsp. vulgare | 3667 | 4969 |
| | | | | [Hordeum vulgare subsp. vulgare] | | | | |
| | | BAJ94589 | 326496253 | predicted protein | 0.766234 | Hordeum vulgare subsp. vulgare | 3668 | 4970 |
| | | | | [Hordeum vulgare subsp. vulgare] | | | | |
| | | EEC81176 | 54362548 | hypothetical protein OsI_24155 | 0.774892 | Oryza sativa Indica Group | 3669 | |
| | | | | [Oryza sativa Indica Group] | | | | |
| | | NP_001058357 | 115469515 | Os06g0677500 [Oryza sativa Japonica Group] | 0.772006 | Oryza sativa Japonica Group | 3670 | 4971 |
| | | | | &gt:gi\|52076622\|dbj\|BAD45523.1\| | | | | |
| | | | | putative Rab geranylgeranyl transferase, | | | | |
| | | | | a subunit [Oryza sativa Japonica Group] | | | | |
| | | | | &gt:gi\|52076908\|dbj\|BAD45920.1\| | | | | |
| | | | | putative Rab geranylgeranyl transferase, | | | | |
| | | | | a subunit [Oryza sativa Japonica Group] | | | | |
| | | | | &gt:gi\|113596397\|dbj\|BAF20271.1\| | | | | |
| | | | | Os06g0677500 [Oryza sativa Japonica Group] | | | | |
| | | | | &gt:gi\|125598230\|gb\|EAZ38010.1\| | | | | |
| | | | | hypothetical protein OsJ_22355 | | | | |
| | | | | [Oryza sativa Japonica Group] | | | | |
| | 2063-2082 | XP_002466382 | 242037974 | hypothetical protein SORBIDRAFT_01g006780 | 1 | Sorghum bicolor | 3671 | 4972 |
| | | | | [Sorghum bicolor] | | | | |
| | | | | &gt:gi\|241920236\|gb\|EER93380.1\| | | | | |
| | | | | hypothetical protein SORBIDRAFT_01g006780 | | | | |
| | | | | [Sorghum bicolor] | | | | |
| | | NP_001147990 | 226508033 | lectin-like receptor kinase 7 [Zea mays] | 0.898975 | Zea mays | 3672 | 4973 |
| | | | | &gt:gi\|195615004\|gb\|ACG29332.1\| | | | | |
| | | | | lectin-like receptor kinase 7 [Zea mays] | | | | |
| | | BAJ98654 | 326498452 | predicted protein | 0.778917 | Hordeum vulgare subsp. vulgare | 3673 | 4974 |
| | | | | [Hordeum vulgare subsp. vulgare] | | | | |
| | | EAY91999 | 54362548 | hypothetical protein OsI_13689 | 0.786237 | Oryza sativa Indica Group | 3674 | |
| | | | | [Oryza sativa Indica Group] | | | | |
| | | ABF99098 | 108705663 | lectin receptor kinase 7, putative, | 0.733529 | Oryza sativa Japonica Group | 3675 | |
| | | | | expressed [Oryza sativa Japonica Group] | | | | |
| | | EAZ28732 | 54398660 | hypothetical protein OsJ_12752 | 0.733529 | Oryza sativa Japonica Group | 3676 | |
| | | | | [Oryza sativa Japonica Group] | | | | |
| | 318-337 | NP_001150139 | 226506903 | 4-methyl-5-thiazole monophosphate biosynthesis | 1 | Zea mays | 3677 | 4975 |
| | | | | protein [Zea mays] | | | | |
| | | | | &gt:gi\|195637064\|gb\|ACG38000.1\| | | | | |
| | | | | 4-methyl-5-thiazole monophosphate biosynthesis | | | | |
| | | | | protein [Zea mays] | | | | |
| | | NP_001130715 | 212274550 | hypothetical protein LOC100191819 | 1.040921 | Zea mays | 3678 | 4976 |
| | | | | [Zea mays] | | | | |
| | | | | &gt:gi\|194689918\|gb\|ACF79043.1\| | | | | |
| | | | | unknown [Zea mays] | | | | |
| | | XP_002457130 | 242055968 | hypothetical protein SORBIDRAFT_03g001750 | 0.879795 | Sorghum bicolor | 3679 | 4977 |
| | | | | [Sorghum bicolor] | | | | |
| | | | | &gt:gi\|241929105\|gb\|EES02250.1\| | | | | |
| | | | | hypothetical protein SORBIDRAFT_03g001750 | | | | |
| | | | | [Sorghum bicolor] | | | | |
| | | XP_002457129 | 242055966 | hypothetical protein SORBIDRAFT_03g001740 | 0.713555 | Sorghum bicolor | 3680 | 4978 |
| | | | | [Sorghum bicolor] | | | | |
| | | | | &gt:gi\|241929104\|gb\|EES02249.1\| | | | | |
| | | | | hypothetical protein SORBIDRAFT_03g001740 | | | | |
| | | | | [Sorghum bicolor] | | | | |
| | 247-266 | ACF84329 | 194700489 | unknown [Zea mays] | 1 | Zea mays | 3681 | 4979 |
| | | NP_001143181 | 226491793 | hypothetical protein LOC100275681 | 0.983505 | Zea mays | 3682 | 4980 |
| | | | | [Zea mays] | | | | |
| | | | | &gt:gi\|195615484\|gb\|ACG29572.1\| | | | | |
| | | | | hypothetical protein [Zea mays] | | | | |
| | | XP_002439532 | 242087398 | hypothetical protein SORBIDRAFT_09g010410 | 0.775258 | Sorghum bicolor | 3683 | 4981 |
| | | | | [Sorghum bicolor] | | | | |
| | | | | &gt:gi\|241944817\|gb\|EES17962.1\| | | | | |
| | | | | hypothetical protein SORBIDRAFT_09g010410 | | | | |
| | | | | [Sorghum bicolor] | | | | |
| | 1371-1390 | XP_002463817 | 242032844 | hypothetical protein SORBIDRAFT_01g006730 | 1 | Sorghum bicolor | 3684 | 4982 |
| | | | | [Sorghum bicolor] | | | | |
| | | | | &gt:gi\|241917671\|gb\|EER90815.1\| | | | | |
| | | | | hypothetical protein SORBIDRAFT_01g006730 | | | | |
| | | | | [Sorghum bicolor] | | | | |
| | | ACF82838 | 238908755 | unknown [Zea mays] | 0.96789 | Zea mays | 3685 | 4983 |
| | | ACG44256 | 195649576 | hypothetical protein [Zea mays] | 0.965596 | Zea mays | 3686 | 4984 |
| | | NP_001141500 | 226490864 | hypothetical protein LOC100273612 | 0.933486 | Zea mays | 3687 | 4985 |
| | | | | [Zea mays] | | | | |
| | | | | &gt:gi\|194704836\|gb\|ACF86502.1\| | | | | |
| | | | | unknown [Zea mays] | | | | |
| | | | | &gt:gi\|194707468\|gb\|ACF87818.1\| | | | | |
| | | | | unknown [Zea mays] | | | | |
| | | EAY92004 | 54362548 | hypothetical protein OsI_13693 | 0.848624 | Oryza sativa Indica Group | 3688 | |
| | | | | [Oryza sativa Indica Group] | | | | |
| | | EAZ28736 | 54398660 | hypothetical protein OsJ_12756 | 0.848624 | Oryza sativa Japonica Group | 3689 | |
| | | | | [Oryza sativa Japonica Group] | | | | |
| | | NP_001051414 | 115455626 | Os03g0773000 [Oryza sativa Japonica Group] | 0.84633 | Oryza sativa Japonica Group | 3690 | 4986 |
| | | | | &gt:gi\|31745235\|gb\|AAP68895.1\| | | | | |
| | | | | unknown protein | | | | |
| | | | | [Oryza sativa Japonica Group] | | | | |
| | | | | &gt:gi\|108711308\|gb\|ABF99103.1\| | | | | |
| | | | | expressed protein | | | | |
| | | | | [Oryza sativa Japonica Group] | | | | |
| | | | | &gt:gi\|113549885\|dbj\|BAF13328.1\| | | | | |
| | | | | Os03g0773000 [Oryza sativa Japonica Group] | | | | |
| | | AAP68894 | 28269488 | unknown protein | 0.768349 | Oryza sativa Japonica Group | 3691 | 4987 |
| | | | | [Oryza sativa Japonica Group] | | | | |
| | | BAJ89011 | 326527918 | predicted protein | 0.729358 | Hordeum vulgare subsp. vulgare | 3692 | 4988 |
| | | | | [Hordeum vulgare subsp. vulgare] | | | | |
| | | NP_001058837 | 115470476 | Os07g0133500 [Oryza sativa Japonica Group] | 0.722477 | Oryza sativa Japonica Group | 3693 | 4989 |
| | | | | &gt:gi\|32352156\|dbj\|BAC78571.1\| | | | | |
| | | | | hypothetical protein | | | | |
| | | | | [Oryza sativa Japonica Group] | | | | |
| | | | | &gt:gi\|34393412\|dbj\|BAC82946.1\| | | | | |
| | | | | unknown protein | | | | |
| | | | | [Oryza sativa Japonica Group] | | | | |
| | | | | &gt:gi\|50509299\|dbj\|BAD30606.1\| | | | | |
| | | | | unknown protein | | | | |
| | | | | [Oryza sativa Japonica Group] | | | | |
| | | | | &gt:gi\|113610373\|dbj\|BAF20751.1\| | | | | |
| | | | | Os07g0133500 [Oryza sativa Japonica Group] | | | | |
| | | | | &gt:gi\|125599028\|gb\|EAZ38604.1\| | | | | |
| | | | | hypothetical protein OsJ_22992 | | | | |
| | | | | [Oryza sativa Japonica Group] | | | | |
| | | | | &gt:gi\|215741158\|dbj\|BAG97653.1\| | | | | |
| | | | | unnamed protein product | | | | |
| | | | | [Oryza sativa Japonica Group] | | | | |
| | 338-357 | XP_002465042 | 242035294 | hypothetical protein SORBIDRAFT_01g03 1090 | 1 | Sorghum bicolor | 3694 | 4990 |
| | | | | [Sorghum bicolor] | | | | |
| | | | | &gt:gi\|241918896\|gb\|EER92040.1\| | | | | |
| | | | | hypothetical protein SORBIDRAFT_01g03 1090 | | | | |
| | | | | [Sorghum bicolor] | | | | |
| | | ACG29187 | 195614713 | subtilisin-like protease precursor | 0.934211 | Zea mays | 3695 | 4991 |
| | | | | [Zea mays] | | | | |
| | | EAY79268 | 54362548 | hypothetical protein OsI_34383 | 0.878947 | Oryza sativa Indica Group | 3696 | |
| | | | | [Oryza sativa Indica Group] | | | | |
| | | NP_001065109 | 115483031 | Os10g0524600 [Oryza sativa Japonica Group] | 0.876316 | Oryza sativa Japonica Group | 3697 | 4992 |
| | | | | &gt:gi\|20146761\|gb\|AAM12497.1\|AC074232_24 | | | | |
| | | | | putative serine protease | | | | |
| | | | | [Oryza sativa Japonica Group] | | | | |
| | | | | &gt:gi\|27311277\|gb\|AAO00703.1\| | | | | |
| | | | | putative serine protease | | | | |
| | | | | [Oryza sativa Japonica Group] | | | | |
| | | | | &gt:gi\|31433153\|gb\|AAP54706.1\| | | | | |
| | | | | Subtilisin N-terminal Region family protein, | | | | |
| | | | | expressed [Oryza sativa Japonica Group] | | | | |
| | | | | &gt:gi\|113639718\|dbj\|BAF27023.1\| | | | | |
| | | | | Os10g0524600 [Oryza sativa Japonica Group] | | | | |
| | | | | &gt:gi\|125575456\|gb\|EAZ16740.1\| | | | | |
| | | | | hypothetical protein OsJ_32216 | | | | |
| | | | | [Oryza sativa Japonica Group] | | | | |
| | | | | &gt:gi\|215697336\|dbj\|BAG91330.1\| | | | | |
| | | | | unnamed protein product | | | | |
| | | | | [Oryza sativa Japonica Group] | | | | |
| | | NP_001048778 | 115450354 | Os03g0119300 [Oryza sativa Japonica Group] | 0.744737 | Oryza sativa Japonica Group | 3698 | 4993 |
| | | | | &gt:gi\|27452907\|gb\|AAO15291.1\| | | | | |
| | | | | Putative serine protease | | | | |
| | | | | [Oryza sativa Japonica Group] | | | | |
| | | | | &gt:gi\|108705882\|gb\|ABF93677.1\| | | | | |
| | | | | Subtilisin N-terminal Region family protein, | | | | |
| | | | | expressed [Oryza sativa Japonica Group] | | | | |
| | | | | &gt:gi\|113547249\|dbj\|BAF10692.1\| | | | | |
| | | | | Os03g0119300 | | | | |
| | | | | [Oryza sativa Japonica Group] | | | | |
| | | XP_002465976 | 242037162 | hypothetical protein SORBIDRAFT_01g049280 | 0.731579 | Sorghum bicolor | 3699 | 4994 |
| | | | | [Sorghum bicolor] | | | | |
| | | | | &gt:gi\|241919830\|gb\|EER92974.1\| | | | | |
| | | | | hypothetical protein SORBIDRAFT_01g049280 | | | | |
| | | | | [Sorghum bicolor] | | | | |
| | | BAK05599 | 326490998 | predicted protein | 0.728947 | Hordeum vulgare subsp. vulgare | 3700 | 4995 |
| | | | | [Hordeum vulgare subsp. vulgare] | | | | |
| | | | | &gt:gi\|326496769\|dbj\|BAJ98411.1\| | | | | |
| | | | | predicted protein | | | | |
| | | | | [Hordeum vulgare subsp. vulgare] | | | | |
| | | | | &gt:gi\|326497201\|dbj\|BAK02185.1\| | | | | |
| | | | | predicted protein | | | | |
| | | | | [Hordeum vulgare subsp. vulgare] | | | | |
| | | ACN30792 | 223973208 | unknown [Zea mays] | 0.715789 | Zea mays | 3701 | 4996 |
| | | NP_001148151 | 226498389 | xylem serine proteinase 1 | 0.714474 | Zea mays | 3702 | 4997 |
| | | | | [Zea mays] | | | | |
| | | | | &gt:gi\|195616146\|gb\|ACG29903.1\| | | | | |
| | | | | xylem serine proteinase 1 precursor | | | | |
| | | | | [Zea mays] | | | | |
| | 2007-2026 | XP_002465627 | 242036464 | hypothetical protein SORBIDRAFT_01g042530 | 1 | Sorghum bicolor | 3703 | 4998 |
| | | | | [Sorghum bicolor] | | | | |
| | | | | &gt:gi\|241919481\|gb\|EER92625.1\| | | | | |
| | | | | hypothetical protein SORBIDRAFT_01g042530 | | | | |
| | | | | [Sorghum bicolor] | | | | |
| | | NP_001159302 | 259490447 | hypothetical protein LOC100304394 | 0.919003 | Zea mays | 3704 | 4999 |
| | | | | [Zea mays] | | | | |
| | | | | &gt:gi\|223943285\|gb\|ACN25726.1\| | | | | |
| | | | | unknown [Zea mays] | | | | |
| | | NP_001049417 | 115451632 | Os03g0222100 [Oryza sativa Japonica Group] | 0.806854 | Oryza sativa Japonica Group | 3705 | 5000 |
| | | | | &gt:gi\|108706910\|gb\|ABF94705.1\| | | | | |
| | | | | MA3 domain-containing protein, putative, | | | | |
| | | | | expressed [Oryza sativa Japonica Group] | | | | |
| | | | | &gt:gi\|113547888\|dbj\|BAF11331.1\| | | | | |
| | | | | Os03g0222100 [Oryza sativa Japonica Group] | | | | |
| | | AAN05329 | 22417315 | Putative topoisomerase | 0.805296 | Oryza sativa Japonica Group | 3706 | 5001 |
| | | | | [Oryza sativa Japonica Group] | | | | |
| | | | | &gt:gi\|125542940\|gb\|EAY89079.1\| | | | | |
| | | | | hypothetical protein OsI_10565 | | | | |
| | | | | [Oryza sativa Indica Group] | | | | |
| | | BAK08048 | 326527546 | predicted protein | 0.816199 | Hordeum vulgare subsp. vulgare | 3707 | 5002 |
| | | | | [Hordeum vulgare subsp. vulgare] | | | | |
| | | EEE58613 | 54398660 | hypothetical protein OsJ_09961 | 0.772586 | Oryza sativa Japonica Group | 3708 | |
| | | | | [Oryza sativa Japonica Group] | | | | |
| | 924-943 | BAJ85289 | 326493655 | predicted protein | 1 | Hordeum vulgare subsp. vulgare | 3709 | 5003 |
| | | | | [Hordeum vulgare subsp. vulgare] | | | | |
| | | NP_001130482 | 212275084 | hypothetical protein LOC100191580 | 0.929688 | Zea mays | 3710 | 5004 |
| | | | | [Zea mays] | | | | |
| | | | | &gt:gi\|194689252\|gb\|ACF78710.1\| | | | | |
| | | | | unknown [Zea mays] | | | | |
| | | | | &gt:gi\|223972733\|gb\|ACN30554.1\| | | | | |
| | | | | unknown [Zea mays] | | | | |
| | | NP_001056670 | 115466141 | Os06g0128200 [Oryza sativa Japonica Group] | 0.904297 | Oryza sativa Japonica Group | 3711 | 5005 |
| | | | | &gt:gi\|75115092\|sp\|Q658I5.1\|LMBD1_ORYSJ | | | | |
| | | | | RecName: Full = LIMR family protein Os06g0128200 | | | | |
| | | | | &gt:gi\|52075611\|dbj\|BAD44782.1\| | | | | |
| | | | | LMBR1 integral membrane family protein-like | | | | |
| | | | | [Oryza sativa Japonica Group] | | | | |
| | | | | &gt:gi\|55296214\|dbj\|BAD67932.1\| | | | | |
| | | | | LMBR1 integral membrane family protein-like | | | | |
| | | | | [Oryza sativa Japonica Group] | | | | |
| | | | | &gt:gi\|113594710\|dbj\|BAF18584.1\| | | | | |
| | | | | Os06g0128200 [Oryza sativa Japonica Group] | | | | |
| | | | | &gt:gi\|215697147\|dbj\|BAG91141.1\| | | | | |
| | | | | unnamed protein product | | | | |
| | | | | [Oryza sativa Japonica Group] | | | | |
| | | | | &gt:gi\|218197487\|gb\|EEC79914.1\| | | | | |
| | | | | hypothetical protein OsI_21464 | | | | |
| | | | | [Oryza sativa Indica Group] | | | | |
| | | | | &gt:gi\|222634886\|gb\|EEE65018.1\| | | | | |
| | | | | hypothetical protein OsJ_19972 | | | | |
| | | | | [Oryza sativa Japonica Group] | | | | |
| | | XP_002873012 | 297810256 | LMBR1 integral membrane family protein | 0.802734 | Arabidopsis lyrata subsp. lyrata | 3712 | 5006 |
| | | | | [Arabidopsis lyrata subsp. lyrata] | | | | |
| | | | | &gt:gi\|297318849\|gb\|EFH49271.1\| | | | | |
| | | | | LMBR1 integral membrane family protein | | | | |
| | | | | [Arabidopsis lyrata subsp. lyrata] | | | | |
| | | XP_002528095 | 255574362 | conserved hypothetical protein [Ricinus communis] | 0.802734 | Ricinus communis | 3713 | 5007 |
| | | | | &gt:gi\|223532484\|gb\|EEF34274.1\| conserved | | | | |
| | | | | hypothetical protein [Ricinus communis] | | | | |
| | | XP_002882590 | 297829415 | hypothetical protein ARALYDRAFT_478196 | 0.806641 | Arabidopsis lyrata subsp. lyrata | 3714 | 5008 |
| | | | | [Arabidopsis lyrata subsp. lyrata] | | | | |
| | | | | &gt:gi\|297328430\|gb\|EFH58849.1\| | | | | |
| | | | | hypothetical protein ARALYDRAFT_478196 | | | | |
| | | | | [Arabidopsis lyrata subsp. lyrata] | | | | |
| | | NP_195766 | 30679270 | LMBR1-like membrane protein | 0.800781 | Arabidopsis thaliana | 3715 | 5009 |
| | | | | [Arabidopsis thaliana] | | | | |
| | | | | &gt:gi\|75181394\|sp\|Q9M028.1\|LMBD2_ARATH | | | | |
| | | | | RecName: Full = LIMR family protein At5g01460 | | | | |
| | | | | &gt:gi\|7320724\|emb\|CAB81929.1\| | | | | |
| | | | | putative protein [Arabidopsis thaliana] | | | | |
| | | | | &gt:gi\|18176296\|gb\|AAL60018.1\| | | | | |
| | | | | unknown protein [Arabidopsis thaliana] | | | | |
| | | | | &gt:gi\|20465353\|gb\|AAM20080.1\| | | | | |
| | | | | unknown protein [Arabidopsis thaliana] | | | | |
| | | | | &gt:gi\|332002964\|gb\|AED90347.1\| | | | | |
| | | | | LMBR1-like membrane protein | | | | |
| | | | | [Arabidopsis thaliana] | | | | |
| | | NP_566338 | 240255304 | LMBR1-like membrane protein | 0.804688 | Arabidopsis thaliana | 3716 | 5010 |
| | | | | [Arabidopsis thaliana] | | | | |
| | | | | &gt:gi\|226789815\|sp\|Q9SR93.2\|LMBD1_ARATH | | | | |
| | | | | RecName: Full = LIMR family protein At3g08930 | | | | |
| | | | | &gt:gi\|14334836\|gb\|AAK59596.1\| | | | | |
| | | | | unknown protein [Arabidopsis thaliana] | | | | |
| | | | | &gt:gi\|24417362\|gb\|AAN60291.1\| | | | | |
| | | | | unknown [Arabidopsis thaliana] | | | | |
| | | | | &gt:gi\|56550703\|gb\|AAV97805.1\| | | | | |
| | | | | At3g08930 [Arabidopsis thaliana] | | | | |
| | | | | &gt:gi\|332641175\|gb\|AEE74696.1\| | | | | |
| | | | | LMBR1-like membrane protein | | | | |
| | | | | [Arabidopsis thaliana] | | | | |
| | | XP_002280330 | 225440691 | PREDICTED: hypothetical protein | 0.796875 | Vitis vinifera | 3717 | 5011 |
| | | | | [Vitis vinifera] | | | | |
| | | | | &gt:gi\|297740207\|emb\|CBI30389.3\| | | | | |
| | | | | unnamed protein product [Vitis vinifera] | | | | |
| | | XP_002323579 | 224140416 | predicted protein [Populus trichocarpa] | 0.796875 | Populus trichocarpa | 3718 | 5012 |
| | | | | &gt:gi\|222868209\|gb\|EEF05340.1\| | | | | |
| | | | | predicted protein [Populus trichocarpa] | | | | |
| | 716-735 | BAJ97749 | 326531489 | predicted protein | 1 | Hordeum vulgare subsp. vulgare | 3719 | 5013 |
| | | | | [Hordeum vulgare subsp. vulgare] | | | | |
| | 714-733 | XP_002456960 | 242055628 | hypothetical protein SORBIDRAFT_03g046410 | 1 | Sorghum bicolor | 3720 | 5014 |
| | | | | [Sorghum bicolor] | | | | |
| | | | | &gt:gi\|241928935\|gb\|EES02080.1\| | | | | |
| | | | | hypothetical protein SORBIDRAFT_03g046410 | | | | |
| | | | | [Sorghum bicolor] | | | | |
| | | NP_001168278 | 293335408 | hypothetical protein LOC100382042 | 0.87905 | Zea mays | 3721 | 5015 |
| | | | | [Zea mays] | | | | |
| | | | | &gt:gi\|223947167\|gb\|ACN27667.1\| | | | | |
| | | | | unknown [Zea mays] | | | | |
| | | BAJ90008 | 326490681 | predicted protein | 0.721382 | Hordeum vulgare subsp. vulgare | 3722 | 5016 |
| | | | | [Hordeum vulgare subsp. vulgare] | | | | |
| | 1011-1030 | NP_001105211 | 162460524 | fructokinase-2 [Zea mays] | 1 | Zea mays | 3723 | 5017 |
| | | | | &gt:gi\|75293603\|sp\|Q6XZ78.1\|SCRK2_MAIZE | | | | |
| | | | | RecName: Full = Fructokinase-2; | | | | |
| | | | | AltName: Full = ZmFRK2 | | | | |
| | | | | &gt:gi\|31652276\|gb\|AAP42806.1\| | | | | |
| | | | | fructokinase 2 [Zea mays] | | | | |
| | | NP_001060837 | 115474480 | Os08g0113100 [Oryza sativa Japonica Group] | 0.907463 | Oryza sativa Japonica Group | 3724 | 5018 |
| | | | | &gt:gi\|122234591\|sp\|Q0J8G4.1\|SCRK2_ORYSJ | | | | |
| | | | | RecName: Full = Fructokinase-2; | | | | |
| | | | | AltName: Full = Fructokinase II; | | | | |
| | | | | AltName: Full = OsFKII | | | | |
| | | | | &gt:gi\|158513662\|sp\|A2YQL4.2\|SCRK2_ORYSI | | | | |
| | | | | RecName: Full = Fructokinase-2; | | | | |
| | | | | AltName: Full = Fructokinase II; | | | | |
| | | | | AltName: Full = OsFKII | | | | |
| | | | | &gt:gi\|16566704\|gb\|AAL26573.1\|AF429947_1 | | | | |
| | | | | putative fructokinase II [Oryza sativa] | | | | |
| | | | | &gt:gi\|32352126\|dbj\|BAC78556.1\| | | | | |
| | | | | fructokinase [Oryza sativa Japonica Group] | | | | |
| | | | | &gt:gi\|42408363\|dbj\|BAD09515.1\| | | | | |
| | | | | putative fructokinase | | | | |
| | | | | [Oryza sativa Japonica Group] | | | | |
| | | | | &gt:gi\|113622806\|dbj\|BAF22751.1\| | | | | |
| | | | | Os08g0113100 [Oryza sativa Japonica Group] | | | | |
| | | | | &gt:gi\|125601970\|gb\|EAZ41295.1\| | | | | |
| | | | | hypothetical protein OsJ_25803 | | | | |
| | | | | [Oryza sativa Japonica Group] | | | | |
| | | | | &gt:gi\|215687214\|dbj\|BAG91779.1\| | | | | |
| | | | | unnamed protein product | | | | |
| | | | | [Oryza sativa Japonica Group] | | | | |
| | | | | &gt:gi\|215708813\|dbj\|BAG94082.1\| | | | | |
| | | | | unnamed protein product | | | | |
| | | | | [Oryza sativa Japonica Group] | | | | |
| | 600-619 | NP_001158910 | 259490644 | hypothetical protein LOC100303804 | 1 | Zea mays | 3725 | 5019 |
| | | | | [Zea mays] | | | | |
| | | | | &gt:gi\|194703158\|gb\|ACF85663.1\| | | | | |
| | | | | unknown [Zea mays] | | | | |
| | 1224-1243 | NP_001183123 | 308044352 | hypothetical protein LOC100501490 | 1 | Zea mays | 3726 | 5020 |
| | | | | [Zea mays] | | | | |
| | | | | &gt:gi\|238009484\|gb\|ACR35777.1\| | | | | |
| | | | | unknown [Zea mays] | | | | |
| | | XP_002436478 | 242091975 | hypothetical protein SORBIDRAFT_10g003410 | 0.758519 | Sorghum bicolor | 3727 | 5021 |
| | | | | [Sorghum bicolor] | | | | |
| | | | | &gt:gi\|241914701\|gb\|EER87845.1\| | | | | |
| | | | | hypothetical protein SORBIDRAFT_10g003410 | | | | |
| | | | | [Sorghum bicolor] | | | | |
| | 1268-1287 | XP_002443799 | 242078060 | hypothetical protein SORBIDRAFT_07g002260 | 1 | Sorghum bicolor | 3728 | 5022 |
| | | | | [Sorghum bicolor] | | | | |
| | | | | &gt:gi\|241940149\|gb\|EES13294.1\| | | | | |
| | | | | hypothetical protein SORBIDRAFT_07g002260 | | | | |
| | | | | [Sorghum bicolor] | | | | |
| | 30-49 | NP_001183250 | 308080805 | hypothetical protein LOC100501641 | 1 | Zea mays | 3729 | 5023 |
| | | | | [Zea mays] | | | | |
| | | | | &gt:gi\|238010326\|gb\|ACR36198.1\| | | | | |
| | | | | unknown [Zea mays] | | | | |
| | 760-779 | NP_001143079 | 226492450 | hypothetical protein LOC100275552 | 1 | Zea mays | 3730 | 5024 |
| | | | | [Zea mays] | | | | |
| | | | | &gt:gi\|195613956\|gb\|ACG28808.1\| | | | | |
| | | | | hypothetical protein [Zea mays] | | | | |
| | 1545-1564 | ACF84241 | 194700313 | unknown [Zea mays] | 1 | Zea mays | 3731 | 5025 |
| | | | | &gt:gi\|194707260\|gb\|ACF87714.1\| | | | | |
| | | | | unknown [Zea mays] | | | | |
| | | NP_001147589 | 226507680 | endo-1,4-beta-xylanase [Zea mays] | 0.969543 | Zea mays | 3732 | 5026 |
| | | | | &gt:gi\|195612376\|gb\|ACG28018.1\| | | | | |
| | | | | endo-1,4-beta-xylanase [Zea mays] | | | | |
| | | XP_002455242 | 242052192 | hypothetical protein SORBIDRAFT_03g006980 | 0.873096 | Sorghum bicolor | 3733 | 5027 |
| | | | | [Sorghum bicolor] | | | | |
| | | | | &gt:gi\|241927217\|gb\|EES00362.1\| | | | | |
| | | | | hypothetical protein SORBIDRAFT_03g006980 | | | | |
| | | | | [Sorghum bicolor] | | | | |
| | | EAY72425 | 54362548 | hypothetical protein OsI_00279 | 0.746193 | Oryza sativa Indica Group | 3734 | |
| | | | | [Oryza sativa Indica Group] | | | | |
| | | EEE53821 | 54398660 | hypothetical protein OsJ_00270 | 0.71912 | Oryza sativa Japonica Group | 3735 | |
| | | | | [Oryza sativa Japonica Group] | | | | |
| | 273-292 | NP_001132755 | 212721503 | hypothetical protein LOC100194242 | 1 | Zea mays | 3736 | 5028 |
| | | | | [Zea mays] | | | | |
| | | | | &gt:gi\|194695312\|gb\|ACF81740.1\| | | | | |
| | | | | unknown [Zea mays] | | | | |
| | | | | &gt:gi\|223946859\|gb\|ACN27513.1\| | | | | |
| | | | | unknown [Zea mays] | | | | |
| | | ACG31916 | 195620171 | RHC1A [Zea mays] | 0.895385 | Zea mays | 3737 | 5029 |
| | | NP_001148878 | 226501599 | LOC100282497 [Zea mays] | 0.84 | Zea mays | 3738 | 5030 |
| | | | | &gt:gi\|195622884\|gb\|ACG33272.1\| | | | | |
| | | | | RHC1A [Zea mays] | | | | |
| | | ACF82310 | 194696451 | unknown [Zea mays] | 0.843077 | Zea mays | 3739 | 5031 |
| | 241-260 | NP_001144625 | 226498917 | hypothetical protein LOC100277643 | 1 | Zea mays | 3740 | 5032 |
| | | | | [Zea mays] | | | | |
| | | | | &gt:gi\|195644788\|gb\|ACG41862.1\| | | | | |
| | | | | hypothetical protein [Zea mays] | | | | |
| | 160-179 | XP_002440874 | 242090082 | hypothetical protein SORBIDRAFT_09g011240 | 1 | Sorghum bicolor | 3741 | 5033 |
| | | | | [Sorghum bicolor] | | | | |
| | | | | &gt:gi\|241946159\|gb\|EES19304.1\| | | | | |
| | | | | hypothetical protein SORBIDRAFT_09g011240 | | | | |
| | | | | [Sorghum bicolor] | | | | |
| | | ACL54333 | 219887916 | unknown [Zea mays] | 0.935484 | Zea mays | 3742 | 5034 |
| | | NP_001152417 | 226533398 | ubiquinone biosynthesis protein ubiB | 0.931276 | Zea mays | 3743 | 5035 |
| | | | | [Zea mays] | | | | |
| | | | | &gt:gi\|195656083\|gb\|ACG47509.1\| | | | | |
| | | | | ubiquinone biosynthesis protein ubiB | | | | |
| | | | | [Zea mays] | | | | |
| | | BAK08115 | 326527680 | predicted protein | 0.851332 | Hordeum vulgare subsp. vulgare | 3744 | 5036 |
| | | | | [Hordeum vulgare subsp. vulgare] | | | | |
| | | EEC78984 | 54362548 | hypothetical protein OsI_19471 | 0.778401 | Oryza sativa Indica Group | 3745 | |
| | | | | [Oryza sativa Indica Group] | | | | |
| | | EEE63256 | 54398660 | hypothetical protein OsJ_18066 | 0.776999 | Oryza sativa Japonica Group | 3746 | |
| | | | | [Oryza sativa Japonica Group] | | | | |
| | | XP_002301879 | 224065602 | predicted protein [Populus trichocarpa] | 0.709677 | Populus trichocarpa | 3747 | 5037 |
| | | | | &gt:gi\|222843605\|gb\|EEE81152.1\| | | | | |
| | | | | predicted protein [Populus trichocarpa] | | | | |
| | | XP_002263052 | 225464715 | PREDICTED: hypothetical protein | 0.737728 | Vitis vinifera | 3748 | 5038 |
| | | | | [Vitis vinifera] | | | | |
| | 150-169 | XP_002453372 | 242064165 | hypothetical protein SORBIDRAFT_04g004820 | 1 | Sorghum bicolor | 3749 | 5039 |
| | | | | [Sorghum bicolor] | | | | |
| | | | | &gt:gi\|241933203\|gb\|EES06348.1\| | | | | |
| | | | | hypothetical protein SORBIDRAFT_04g004820 | | | | |
| | | | | [Sorghum bicolor] | | | | |
| | | EEE56406 | 54398660 | hypothetical protein OsJ_05563 | 0.867458 | Oryza sativa Japonica Group | 3750 | |
| | | | | [Oryza sativa Japonica Group] | | | | |
| Predicted | 430-449 | XP_002439337 | 242087008 | hypothetical protein SORBIDRAFT_09g004620 | 1 | Sorghum bicolor | 3751 | 5040 |
| zma | | | | [Sorghum bicolor] | | | | |
| mir | | | | &gt:gi\|241944622\|gb\|EES17767.1\| | | | | |
| 50483 | | | | hypothetical protein SORBIDRAFT_09g004620 | | | | |
| | | | | [Sorghum bicolor] | | | | |
| | | ACG35753 | 195627845 | macrophage erythroblast attacher | 0.950617 | Zea mays | 3752 | 5041 |
| | | | | [Zea mays] | | | | |
| | | | | &gt:gi\|223949645\|gb\|ACN28906.1\| | | | | |
| | | | | unknown [Zea mays] | | | | |
| | | NP_001054719 | 115462238 | Os05g0160100 [Oryza sativa Japonica Group] | 0.814815 | Oryza sativa Japonica Group | 3753 | 5042 |
| | | | | &gt:gi\|51038108\|gb\|AAT93911.1\| unknown | | | | |
| | | | | protein [Oryza sativa Japonica Group] | | | | |
| | | | | &gt:gi\|55168055\|gb\|AAV43923.1\| unknown | | | | |
| | | | | protein [Oryza sativa Japonica Group] | | | | |
| | | | | &gt:gi\|113578270\|dbj\|BAF16633.1\| | | | | |
| | | | | Os05g0160100 [Oryza sativa Japonica Group] | | | | |
| | | | | &gt:gi\|125550933\|gb\|EAY96642.1\| | | | | |
| | | | | hypothetical protein OsI_18556 | | | | |
| | | | | [Oryza sativa Indica Group] | | | | |
| | | | | &gt:gi\|215707056\|dbj\|BAG93516.1\| | | | | |
| | | | | unnamed protein product | | | | |
| | | | | [Oryza sativa Japonica Group] | | | | |
| | | | | &gt:gi\|222630285\|gb\|EEE62417.1\| | | | | |
| | | | | hypothetical protein OsJ_17208 | | | | |
| | | | | [Oryza sativa Japonica Group] | | | | |
| | | BAK01850 | 326489738 | predicted protein | 0.859259 | Hordeum vulgare subsp. vulgare | 3754 | 5043 |
| | | | | [Hordeum vulgare subsp. vulgare] | | | | |
| | 898-917 | ACF80701 | 194693233 | unknown [Zea mays] | 1 | Zea mays | 3755 | 5044 |
| | | NP_001141965 | 226493338 | hypothetical protein LOC100274114 | 1 | Zea mays | 3756 | 5045 |
| | | | | [Zea mays] | | | | |
| | | | | &gt:gi\|194706606\|gb\|ACF87387.1\| | | | | |
| | | | | unknown [Zea mays] | | | | |
| | 150-169 | XP_002467057 | 242039324 | hypothetical protein SORBIDRAFT_01g018900 | 1 | Sorghum bicolor | 3757 | 5046 |
| | | | | [Sorghum bicolor] | | | | |
| | | | | &gt:gi\|241920911\|gb\|EER94055.1\| | | | | |
| | | | | hypothetical protein SORBIDRAFT_01g018900 | | | | |
| | | | | [Sorghum bicolor] | | | | |
| | | ACL52685 | 219884620 | unknown [Zea mays] | 0.922222 | Zea mays | 3758 | 5047 |
| | | NP_001150075 | 226501115 | lysM domain containing protein | 0.919444 | Zea mays | 3759 | 5048 |
| | | | | [Zea mays] | | | | |
| | | | | &gt:gi\|195636494\|gb\|ACG37715.1\| | | | | |
| | | | | lysM domain containing protein | | | | |
| | | | | [Zea mays] | | | | |
| | | NP_001152252 | 226496336 | lysM domain containing protein | 0.947222 | Zea mays | 3760 | 5049 |
| | | | | [Zea mays] | | | | |
| | | | | &gt:gi\|195654297\|gb\|ACG46616.1\| | | | | |
| | | | | lysM domain containing protein | | | | |
| | | | | [Zea mays] | | | | |
| | | NP_001064897 | 115482607 | Os10g0485500 [Oryza sativa Japonica Group] | 0.772222 | Oryza sativa Japonica Group | 3761 | 5050 |
| | | | | &gt:gi\|18087889\|gb\|AAL59043.1\|AC087182_26 | | | | |
| | | | | unknown protein [Oryza sativa Japonica Group] | | | | |
| | | | | &gt:gi\|31432752\|gb\|AAP54345.1\| | | | | |
| | | | | LysM domain containing protein, | | | | |
| | | | | expressed [Oryza sativa Japonica Group] | | | | |
| | | | | &gt:gi\|113639506\|dbj\|BAF26811.1\| | | | | |
| | | | | Os10g0485500 [Oryza sativa Japonica Group] | | | | |
| | | | | &gt:gi\|125532413\|gb\|EAY78978.1\| | | | | |
| | | | | hypothetical protein OsI_34084 | | | | |
| | | | | [Oryza sativa Indica Group] | | | | |
| | 691-710 | XP_002455492 | 242052692 | hypothetical protein SORBIDRAFT_03g011880 | 1 | Sorghum bicolor | 3762 | 5051 |
| | | | | [Sorghum bicolor] | | | | |
| | | | | &gt:gi\|229609765\|gb\|ACQ83498.1\| | | | | |
| | | | | CBL-interacting protein kinase 21 | | | | |
| | | | | [Sorghum bicolor] | | | | |
| | | | | &gt:gi\|241927467\|gb\|EES00612.1\| | | | | |
| | | | | hypothetical protein SORBIDRAFT_03g011880 | | | | |
| | | | | [Sorghum bicolor] | | | | |
| | | NP_001105967 | 162461846 | putative protein kinase [Zea mays] | 0.933045 | Zea mays | 3763 | 5052 |
| | | | | &gt:gi\|120400397\|gb\|ABM21449.1\| | | | | |
| | | | | putative protein kinase [Zea mays] | | | | |
| | | ACG35130 | 195626599 | CBL-interacting serine/threonine-protein | 0.922246 | Zea mays | 3764 | 5053 |
| | | | | kinase 1 [Zea mays] | | | | |
| | | NP_001042792 | 115436067 | Os01g0292200 [Oryza sativa Japonica Group] | 0.866091 | Oryza sativa Japonica Group | 3765 | 5054 |
| | | | | &gt:gi\|75334984\|sp\|Q9LGV5.1\|CIPK1_ORYSJ | | | | |
| | | | | RecName: Full = CBL-interacting protein kinase 1; | | | | |
| | | | | AltName: Full = OsCIPK01 | | | | |
| | | | | &gt:gi\|8468028\|dbj\|BAA96628.1\| | | | | |
| | | | | putative CBL-interacting protein kinase 1 | | | | |
| | | | | [Oryza sativa Japonica Group] | | | | |
| | | | | &gt:gi\|113532323\|dbj\|BAF04706.1\| | | | | |
| | | | | Os01g0292200 [Oryza sativa Japonica Group] | | | | |
| | | | | &gt:gi\|189099603\|gb\|ACD76973.1\| | | | | |
| | | | | CBL-interactingprotein kinase 1 | | | | |
| | | | | [Oryza sativa Japonica Group] | | | | |
| | | | | &gt:gi\|215686723\|dbj\|BAG89573.1\| | | | | |
| | | | | unnamed protein product | | | | |
| | | | | [Oryza sativa Japonica Group] | | | | |
| | | | | &gt:gi\|222618247\|gb\|EEE54379.1\| | | | | |
| | | | | hypothetical protein OsJ_01395 | | | | |
| | | | | [Oryza sativa Japonica Group] | | | | |
| | | NP_001054578 | 115461956 | Os08g0136200 [Oryza sativa Japonica Group] | 0.74514 | Oryza sativa Japonica Group | 3766 | 5055 |
| | | | | &gt:gi\|75326492\|sp\|Q75L42.1\|CIPKH_ORYSJ | | | | |
| | | | | RecName: Full = CBL-interacting protein kinase 17; | | | | |
| | | | | AltName: Full = OsCIPK17 | | | | |
| | | | | &gt:gi\|46485791\|gb\|AAS98416.1\| unknown | | | | |
| | | | | protein [Oryza sativa Japonica Group] | | | | |
| | | | | &gt:gi\|51038254\|gb\|AAT94057.1\| unknown | | | | |
| | | | | protein [Oryza sativa Japonica Group] | | | | |
| | | | | &gt:gi\|113578129\|dbj\|BAF16492.1\| | | | | |
| | | | | Os05g0136200 [Oryza sativa Japonica Group] | | | | |
| | | | | &gt:gi\|189099617\|gb\|ACD76980.1\| | | | | |
| | | | | CBL-interacting protein kinase 17 | | | | |
| | | | | [Oryza sativa Japonica Group] | | | | |
| | | | | &gt:gi\|222630113\|gb\|EEE62245.1\| | | | | |
| | | | | hypothetical protein OsJ_17032 | | | | |
| | | | | [Oryza sativa Japonica Group] | | | | |
| | | EEC78476 | 54362548 | hypothetical protein OsI_18365 | 0.74514 | Oryza sativa Indica Group | 3767 | |
| | | | | [Oryza sativa Indica Group] | | | | |
| | | BAJ96678 | 326519357 | predicted protein | 0.714903 | Hordeum vulgare subsp. vulgare | 3768 | 5056 |
| | | | | [Hordeum vulgare subsp. vulgare] | | | | |
| | 361-380 | NP_001142046 | 226531335 | hypothetical protein LOC100274202 | 1 | Zea mays | 3769 | 5057 |
| | | | | [Zea mays] | | | | |
| | | | | &gt:gi\|194706888\|gb\|ACF87528.1\| | | | | |
| | | | | unknown [Zea mays] | | | | |
| | | ACG42550 | 195646163 | F-box domain containing protein | 0.755501 | Zea mays | 3770 | 5058 |
| | | | | [Zea mays] | | | | |
| | | XP_002441523 | 242091380 | hypothetical protein SORBIDRAFT_09g028610 | 0.765281 | Sorghum bicolor | 3771 | 5059 |
| | | | | [Sorghum bicolor] | | | | |
| | | | | &gt:gi\|241946808\|gb\|EES19953.1\| | | | | |
| | | | | hypothetical protein SORBIDRAFT_09g028610 | | | | |
| | | | | [Sorghum bicolor] | | | | |
| | 123-142 | XP_002451328 | 242072102 | hypothetical protein SORBIDRAFT_05g027870 | 1 | Sorghum bicolor | 3772 | 5060 |
| | | | | [Sorghum bicolor] | | | | |
| | | | | &gt:gi\|241937171\|gb\|EES10316.1\| | | | | |
| | | | | hypothetical protein SORBIDRAFT_05g027870 | | | | |
| | | | | [Sorghum bicolor] | | | | |
| | | NP_001104921 | 162458160 | ribulose bisphosphate carboxylase/oxygenase activase, | 0.915909 | Zea mays | 3773 | 5061 |
| | | | | chloroplastic precursor [Zea mays] | | | | |
| | | | | &gt:gi\|29429152\|sp\|Q9ZT00.3\|RCA_MAIZE | | | | |
| | | | | RecName: Full = Ribulose bisphosphate | | | | |
| | | | | carboxylase/oxygenase activase, chloroplastic; | | | | |
| | | | | Short = RA; Short = RuBisCO activase; | | | | |
| | | | | Flags: Precursor | | | | |
| | | | | &gt:gi\|19855034\|gb\|AAC97932.3\| | | | | |
| | | | | ribulose-1,5-bisphosphate carboxylase/oxygenase | | | | |
| | | | | activase precursor [Zea mays] | | | | |
| | | | | &gt:gi\|195620038\|gb\|ACG31849.1\| | | | | |
| | | | | ribulose bisphosphate carboxylase/oxygenase | | | | |
| | | | | activase [Zea mays] | | | | |
| | | | | &gt:gi\|313574198\|dbj\|BAJ41042.1\| | | | | |
| | | | | ribulose-1,5-bisphosphate carboxylase/oxygenase | | | | |
| | | | | activase small isoform | | | | |
| | | | | [Zea mays] | | | | |
| | | ACN28387 | 223948606 | unknown [Zea mays] | 0.820455 | Zea mays | 3774 | 5062 |
| | | BAA97584 | 8918360 | RuBisCO activase small isoform precursor | 0.834091 | Oryza sativa | 3775 | 5063 |
| | | | | [Oryza sativa] | | | | |
| | | | | &gt:gi\|62733169\|gb\|AAX95286.1\| | | | | |
| | | | | RuBisCO activase small isoform precursor | | | | |
| | | | | [Oryza sativa Japonica Group] | | | | |
| | | | | &gt:gi\|77552726\|gb\|ABA95523.1\| | | | | |
| | | | | Ribulose bisphosphate carboxylase/oxygenase | | | | |
| | | | | activase, chloroplast precursor, putative, expressed | | | | |
| | | | | [Oryza sativa Japonica Group] | | | | |
| | | | | &gt:gi\|215694316\|dbj\|BAG89309.1\| | | | | |
| | | | | unnamed protein product | | | | |
| | | | | [Oryza sativa Japonica Group] | | | | |
| | | ABG22614 | 108863896 | Ribulose bisphosphate carboxylase/oxygenase | 0.834091 | Oryza sativa Japonica Group | 3776 | |
| | | | | activase, chloroplast precursor, putative, expressed | | | | |
| | | | | [Oryza sativa Japonica Group] | | | | |
| | | P93431 | | RecName: Full = Ribulose bisphosphate | 0.834091 | Oryza sativa (Japonica cultivar-group) | 3777 | |
| | | | | carboxylase/oxygenase activase, chloroplastic; | | | | |
| | | | | Short = RA; Short = RuBisCO activase; | | | | |
| | | | | Flags: Precursor | | | | |
| | | | | &gt:gi\|8918359\|dbj\|BAA97583.1\| | | | | |
| | | | | RuBisCO activase large isoform precursor | | | | |
| | | | | [Oryza sativa (Japonica cultivar-group)] | | | | |
| | | | | &gt:gi\|32352158\|dbj\|BAC78572.1\| | | | | |
| | | | | ribulose-bisphosphate carboxylase activase large | | | | |
| | | | | isoform precursor protein | | | | |
| | | | | [Oryza sativa Japonica Group] | | | | |
| | | | | &gt:gi\|77552725\|gb\|ABA95522.1\| | | | | |
| | | | | Ribulose bisphosphate carboxylase/oxygenase | | | | |
| | | | | activase, chloroplast precursor, putative, expressed | | | | |
| | | | | [Oryza sativa Japonica Group] | | | | |
| | | | | &gt:gi\|125578108\|gb\|EAZ19330.1\| | | | | |
| | | | | hypothetical protein OsJ_34880 | | | | |
| | | | | [Oryza sativa Japonica Group] | | | | |
| | | | | &gt:gi\|218186228\|gb\|EEC68655.1\| | | | | |
| | | | | hypothetical protein OsI_37096 | | | | |
| | | | | [Oryza sativa Indica Group] | | | | |
| | | AAC28134 | 3377792 | ribulose-1,5-bisphosphate carboxylase/oxygenase | 0.834091 | Oryza sativa Japonica Group | 3778 | 5064 |
| | | | | activase [Oryza sativa Japonica Group] | | | | |
| | | AAF71272 | 7960276 | ribulose bisphosphate carboxylase activase | 0.818182 | Triticum aestivum | 3779 | 5065 |
| | | | | B [Triticum aestivum] | | | | |
| | | AAP83928 | 32481062 | Rubisco activase beta form precursor | 0.815909 | Deschampsia antarctica | 3780 | 5066 |
| | | | | [Deschampsia antarctica] | | | | |
| | | AAH29790 | 20987526 | Unknown (protein for MGC: 35458) | 0.809091 | Homo sapiens | 3781 | 5067 |
| | | | | [Homo sapiens] | | | | |
| | 1071-1090 | XP_002441513 | 242091360 | hypothetical protein SORBIDRAFT_09g028410 | 1 | Sorghum bicolor | 3782 | 5068 |
| | | | | [Sorghum bicolor] | | | | |
| | | | | &gt:gi\|241946798\|gb\|EES19943.1\| | | | | |
| | | | | hypothetical protein SORBIDRAFT_09g028410 | | | | |
| | | | | [Sorghum bicolor] | | | | |
| | | ACF85297 | 194702425 | unknown [Zea mays] | 0.977901 | Zea mays | 3783 | 5069 |
| | | NP_001149016 | 226510213 | dnaJ subfamily B member 5 [Zea mays] | 0.972376 | Zea mays | 3784 | 5070 |
| | | | | &gt:gi\|195624008\|gb\|ACG33834.1\| | | | | |
| | | | | dnaJ subfamily B member 5 [Zea mays] | | | | |
| | | NP_001168528 | 293332110 | hypothetical protein LOC100382308 | 0.881215 | Zea mays | 3785 | 5071 |
| | | | | [Zea mays] | | | | |
| | | | | &gt:gi\|223948919\|gb\|ACN28543.1\| | | | | |
| | | | | unknown [Zea mays] | | | | |
| | | NP_001056320 | 115465440 | Os05g0562300 [Oryza sativa Japonica Group] | 0.90884 | Oryza sativa Japonica Group | 3786 | 5072 |
| | | | | &gt:gi\|51854270\|gb\|AAU10651.1\| | | | | |
| | | | | &apos:putative; heat shock protein, hsp40&apos: | | | | |
| | | | | [Oryza sativa Japonica Group] | | | | |
| | | | | &gt:gi\|113579871\|dbj\|BAF18234.1\| | | | | |
| | | | | Os05g0562300 [Oryza sativa Japonica Group] | | | | |
| | | | | &gt:gi\|215695218\|dbj\|BAG90409.1\| | | | | |
| | | | | unnamed protein product | | | | |
| | | | | [Oryza sativa Japonica Group] | | | | |
| | | | | &gt:gi\|222632556\|gb\|EEE64688.1\| | | | | |
| | | | | hypothetical protein OsJ_19543 | | | | |
| | | | | [Oryza sativa Japonica Group] | | | | |
| | | EAY99019 | 54362548 | hypothetical protein OsI_20977 | 0.906077 | Oryza sativa Indica Group | 3787 | |
| | | | | [Oryza sativa Indica Group] | | | | |
| | | XP_002278979 | 225443601 | PREDICTED: hypothetical protein | 0.767956 | Vitis vinifera | 3788 | 5073 |
| | | | | [Vitis vinifera] | | | | |
| | | ACU18092 | 255635479 | unknown [Glycine max] | 0.773481 | Glycine max | 3789 | 5074 |
| | | XP_002886287 | 297836809 | DNAJ heat shock family protein | 0.745856 | Arabidopsis lyrata subsp. lyrata | 3790 | 5075 |
| | | | | [Arabidopsis lyrata subsp. lyrata] | | | | |
| | | | | &gt:gi\|297332127\|gb\|EFH62546.1\| | | | | |
| | | | | DNAJ heat shock family protein | | | | |
| | | | | [Arabidopsis lyrata subsp. lyrata] | | | | |
| | | XP_002509430 | 255536726 | Protein SIS1, putative [Ricinus communis] | 0.756906 | Ricinus communis | 3791 | 5076 |
| | | | | &gt:gi\|223549329\|gb\|EEF50817.1\| | | | | |
| | | | | Protein SIS1, putative [Ricinus communis] | | | | |
| | 174-193 | XP_002457615 | 242056938 | hypothetical protein SORBIDRAFT_03g010370 | 1 | Sorghum bicolor | 3792 | 5077 |
| | | | | [Sorghum bicolor] | | | | |
| | | | | &gt:gi\|241929590\|gb\|EES02735.1\| | | | | |
| | | | | hypothetical protein SORBIDRAFT_03g010370 | | | | |
| | | | | [Sorghum bicolor] | | | | |
| | | ACG48423 | 195657910 | hypothetical protein [Zea mays] | 0.943478 | Zea mays | 3793 | 5078 |
| | | NP_001145494 | 226503300 | hypothetical protein LOC100278893 | 0.943478 | Zea mays | 3794 | 5079 |
| | | | | [Zea mays] | | | | |
| | | | | &gt:gi\|195657081\|gb\|ACG48008.1\| | | | | |
| | | | | hypothetical protein [Zea mays] | | | | |
| | | EAZ11347 | 54398660 | hypothetical protein OsJ_01214 | 0.743478 | Oryza sativa Japonica Group | 3795 | |
| | | | | [Oryza sativa Japonica Group] | | | | |
| | | NP_001042671 | 115435825 | Os01g0265700 [Oryza sativa Japonica Group] | 0.752174 | Oryza sativa Japonica Group | 3796 | 5080 |
| | | | | &gt:gi\|56783773\|dbj\|BAD81185.1\| unknown | | | | |
| | | | | protein [Oryza sativa Japonica Group] | | | | |
| | | | | &gt:gi\|113532202\|dbj\|BAF04585.1\| | | | | |
| | | | | Os01g0265700 [Oryza sativa Japonica Group] | | | | |
| | | | | &gt:gi\|125525302\|gb\|EAY73416.1\| | | | | |
| | | | | hypothetical protein OsI_01298 | | | | |
| | | | | [Oryza sativa Indica Group] | | | | |
| | | | | &gt:gi\|215766960\|dbj\|BAG99188.1\| | | | | |
| | | | | unnamed protein product | | | | |
| | | | | [Oryza sativa Japonica Group] | | | | |
| | | BAK07146 | 326515799 | predicted protein | 0.713043 | Hordeum vulgare subsp. vulgare | 3797 | 5081 |
| | | | | [Hordeum vulgare subsp. vulgare] | | | | |
| | 912-931 | NP_001151603 | 226509921 | VAMP protein SEC22 [Zea mays] | 1 | Zea mays | 3798 | 5082 |
| | | | | &gt:gi\|195648052\|gb\|ACG43494.1\| | | | | |
| | | | | VAMP protein SEC22 [Zea mays] | | | | |
| | | NP_001150617 | 226492448 | VAMP protein SEC22 [Zea mays] | 0.890688 | Zea mays | 3799 | 5083 |
| | | | | &gt:gi\|195640598\|gb\|ACG39767.1\| | | | | |
| | | | | VAMP protein SEC22 [Zea mays] | | | | |
| | | ACN30757 | 223973138 | unknown [Zea mays] | 0.874494 | Zea mays | 3800 | 5084 |
| | | BAK04669 | 326529444 | predicted protein | 0.700405 | Hordeum vulgare subsp. vulgare | 3801 | 5085 |
| | | | | [Hordeum vulgare subsp. vulgare] | | | | |
| | | NP_001052766 | 115458331 | Os04g0416700 [Oryza sativa Japonica Group] | 0.704453 | Oryza sativa Japonica Group | 3802 | 5086 |
| | | | | &gt:gi\|38344036\|emb\|CAE01528.2\| | | | | |
| | | | | OJ991214_12.17 [Oryza sativa Japonica Group] | | | | |
| | | | | &gt:gi\|39545714\|emb\|CAD40930.3\| | | | | |
| | | | | OSJNBa0033G16.4 [Oryza sativa Japonica Group] | | | | |
| | | | | &gt:gi\|113564337\|dbj\|BAF14680.1\| | | | | |
| | | | | Os04g0416700 [Oryza sativa Japonica Group] | | | | |
| | | | | &gt:gi\|116310806\|emb\|CAH67596.1\| | | | | |
| | | | | OSIGBa0092M08.8 [Oryza sativa Indica Group] | | | | |
| | | | | &gt:gi\|125548247\|gb\|EAY94069.1\| | | | | |
| | | | | hypothetical protein OsI_15845 | | | | |
| | | | | [Oryza sativa Indica Group] | | | | |
| | | | | &gt:gi\|125590354\|gb\|EAZ30704.1\| | | | | |
| | | | | hypothetical protein OsJ_14762 | | | | |
| | | | | [Oryza sativa Japonica Group] | | | | |
| | | | | &gt:gi\|215686964\|dbj\|BAG90834.1\| | | | | |
| | | | | unnamed protein product | | | | |
| | | | | [Oryza sativa Japonica Group] | | | | |
| | 387-406 | XP_002444876 | 242080214 | hypothetical protein SORBIDRAFT_07g000760 | 1 | Sorghum bicolor | 3803 | 5087 |
| | | | | [Sorghum bicolor] | | | | |
| | | | | &gt:gi\|241941226\|gb\|EES14371.1\| | | | | |
| | | | | hypothetical protein SORBIDRAFT_07g000760 | | | | |
| | | | | [Sorghum bicolor] | | | | |
| | | NP_001148818 | 226531679 | shwachman-Bodian-Diamond syndrome protein | 0.95493 | Zea mays | 3804 | 5088 |
| | | | | [Zea mays] | | | | |
| | | | | &gt:gi\|194708114\|gb\|ACF88141.1\| | | | | |
| | | | | unknown [Zea mays] | | | | |
| | | | | &gt:gi\|195622368\|gb\|ACG33014.1\| | | | | |
| | | | | shwachman-Bodian-Diamond syndrome protein | | | | |
| | | | | [Zea mays] | | | | |
| | | NP_001060798 | 115474402 | Os08g0107500 [Oryza sativa Japonica Group] | 0.850704 | Oryza sativa Japonica Group | 3805 | 5089 |
| | | | | &gt:gi\|42408234\|dbj\|BAD09391.1\| | | | | |
| | | | | putative Shwachman-Bodian-Diamond syndrome protein | | | | |
| | | | | [Oryza sativa Japonica Group] | | | | |
| | | | | &gt:gi\|113622767\|dbj\|BAF22712.1\| | | | | |
| | | | | Os08g0107500 [Oryza sativa Japonica Group] | | | | |
| | | | | &gt:gi\|215693786\|dbj\|BAG88985.1\| | | | | |
| | | | | unnamed protein product [Oryza sativa Japonica Group] | | | | |
| | | | | &gt:gi\|218200357\|gb\|EEC82784.1\| | | | | |
| | | | | hypothetical protein OsI_27530 | | | | |
| | | | | [Oryza sativa Indica Group] | | | | |
| | | | | &gt:gi\|222639780\|gb\|EEE67912.1\| | | | | |
| | | | | hypothetical protein OsJ_25763 | | | | |
| | | | | [Oryza sativa Japonica Group] | | | | |
| | | BAJ88850 | 326525606 | predicted protein | 0.785915 | Hordeum vulgare subsp. vulgare | 3806 | 5090 |
| | | | | [Hordeum vulgare subsp. vulgare] | | | | |
| | | | | &gt:gi\|326529251\|dbj\|BAK01019.1\| | | | | |
| | | | | predicted protein | | | | |
| | | | | [Hordeum vulgare subsp. vulgare] | | | | |
| | 231-250 | NP_001142230 | 226530616 | hypothetical protein LOC100274398 | 1 | Zea mays | 3807 | 5091 |
| | | | | [Zea mays] | | | | |
| | | | | &gt:gi\|194707712\|gb\|ACF87940.1\| | | | | |
| | | | | unknown [Zea mays] | | | | |
| | | | | &gt:gi\|323388581\|gb\|ADX60095.1\| | | | | |
| | | | | AP2-EREBP transcription factor | | | | |
| | | | | [Zea mays] | | | | |
| | | NP_001149871 | 226497003 | ethylene responsive protein [Zea mays] | 0.966667 | Zea mays | 3808 | 5092 |
| | | | | &gt:gi\|195635171\|gb\|ACG37054.1\| | | | | |
| | | | | ethylene responsive protein [Zea mays] | | | | |
| | | XP_002448280 | 242076687 | hypothetical protein SORBIDRAFT_06g024355 | 0.818519 | Sorghum bicolor | 3809 | 5093 |
| | | | | [Sorghum bicolor] | | | | |
| | | | | &gt:gi\|241939463\|gb\|EES12608.1\| | | | | |
| | | | | hypothetical protein SORBIDRAFT_06g024355 | | | | |
| | | | | [Sorghum bicolor] | | | | |
| | | NP_001152050 | 226498033 | ethylene-responsive transcription factor 2 | 0.87037 | Zea mays | 3810 | 5094 |
| | | | | [Zea mays] | | | | |
| | | | | &gt:gi\|195652151\|gb\|ACG45543.1\| | | | | |
| | | | | ethylene-responsive transcription factor 2 | | | | |
| | | | | [Zea mays] | | | | |
| | | ABQ52686 | 148009083 | ethylene-responsive factor | 0.77037 | Thinopyrum intermedium | 3811 | 5095 |
| | | | | [Thinopyrum intermedium] | | | | |
| | | ABQ52687 | 148009101 | pathogen-inducible transcription factor | 0.766667 | Triticum aestivum | 3812 | 5096 |
| | | | | ERF3 [Triticum aestivum] | | | | |
| | | NP_001053474 | 297603127 | Os04g0546800 [Oryza sativa Japonica Group] | 0.740741 | Oryza sativa Japonica Group | 3813 | 5097 |
| | | | | &gt:gi\|70663974\|emb\|CAD41472.3\| | | | | |
| | | | | OSJNBa0079A21.16 [Oryza sativa Japonica Group] | | | | |
| | | | | &gt:gi\|113565045\|dbj\|BAF15388.1\| | | | | |
| | | | | Os04g0546800 [Oryza sativa Japonica Group] | | | | |
| | | | | &gt:gi\|117501525\|gb\|ABK34954.1\| | | | | |
| | | | | development related ERF protein | | | | |
| | | | | [Oryza sativa Japonica Group] | | | | |
| | | | | &gt:gi\|215769250\|dbj\|BAH01479.1\| | | | | |
| | | | | unnamed protein product | | | | |
| | | | | [Oryza sativa Japonica Group] | | | | |
| | | CAH67263 | 116310240 | OSIGBa0101C23.15 [Oryza sativa Indica Group] | 0.740741 | Oryza sativa Indica Group | 3814 | 5098 |
| | | EAY95058 | 54362548 | hypothetical protein OsI_16873 | 0.740741 | Oryza sativa Indica Group | 3815 | |
| | | | | [Oryza sativa Indica Group] | | | | |
| | | BAK05766 | 326491332 | predicted protein | 0.737037 | Hordeum vulgare subsp. vulgare | 3816 | 5099 |
| | | | | [Hordeum vulgare subsp. vulgare] | | | | |
| | 345-364 | XP_002440768 | 242089870 | hypothetical protein SORBIDRAFT_09g006240 | 1 | Sorghum bicolor | 3817 | 5100 |
| | | | | [Sorghum bicolor] | | | | |
| | | | | &gt:gi\|241946053\|gb\|EES19198.1\| | | | | |
| | | | | hypothetical protein SORBIDRAFT_09g006240 | | | | |
| | | | | [Sorghum bicolor] | | | | |
| | | NP_001150209 | 226531775 | prenylated rab acceptor family protein | 0.918367 | Zea mays | 3818 | 5101 |
| | | | | [Zea mays] | | | | |
| | | | | &gt:gi\|195637576\|gb\|ACG38256.1\| | | | | |
| | | | | prenylated rab acceptor family protein | | | | |
| | | | | [Zea mays] | | | | |
| | | BAJ94157 | 326490166 | predicted protein | 0.714286 | Hordeum vulgare subsp. vulgare | 3819 | 5102 |
| | | | | [Hordeum vulgare subsp. vulgare] | | | | |
| | | AAT44309 | 37719165 | hypothetical protein | 0.739796 | Oryza sativa Japonica Group | 3820 | 5103 |
| | | | | [Oryza sativa Japonica Group] | | | | |
| | 1398-1417 | XP_002441339 | 242091012 | hypothetical protein SORBIDRAFT_09g024710 | 1 | Sorghum bicolor | 3821 | 5104 |
| | | | | [Sorghum bicolor] | | | | |
| | | | | &gt:gi\|241946624\|gb\|EES19769.1\| | | | | |
| | | | | hypothetical protein SORBIDRAFT_09g024710 | | | | |
| | | | | [Sorghum bicolor] | | | | |
| | | NP_001055967 | 115464734 | Os05g0500900 [Oryza sativa Japonica Group] | 0.79289 | Oryza sativa Japonica Group | 3822 | 5105 |
| | | | | &gt:gi\|75113903\|sp\|Q60EJ6.1\|GH34_ORYSJ | | | | |
| | | | | RecName: Full = Probable indole-3-acetic | | | | |
| | | | | acid-amido synthetase GH3.4; | | | | |
| | | | | AltName: Full = Auxin-responsive GH3-like protein 4; | | | | |
| | | | | Short = OsGH3-4 | | | | |
| | | | | &gt:gi\|53749366\|gb\|AAU90225.1\| | | | | |
| | | | | putative auxin-responsive protein GH3 | | | | |
| | | | | [Oryza sativa Japonica Group] | | | | |
| | | | | &gt:gi\|113579518\|dbj\|BAF17881.1\| | | | | |
| | | | | Os05g0500900 [Oryza sativa Japonica Group] | | | | |
| | | | | &gt:gi\|125552879\|gb\|EAY98588.1\| | | | | |
| | | | | hypothetical protein OsI_20501 | | | | |
| | | | | [Oryza sativa Indica Group] | | | | |
| | | | | &gt:gi\|222632129\|gb\|EEE64261.1\| | | | | |
| | | | | hypothetical protein OsJ_19094 | | | | |
| | | | | [Oryza sativa Japonica Group] | | | | |
| | | XP_002456458 | 242054624 | hypothetical protein SORBIDRAFT_03g036680 | 0.772798 | Sorghum bicolor | 3823 | 5106 |
| | | | | [Sorghum bicolor] | | | | |
| | | | | &gt:gi\|241928433\|gb\|EES01578.1\| | | | | |
| | | | | hypothetical protein SORBIDRAFT_03g036680 | | | | |
| | | | | [Sorghum bicolor] | | | | |
| | | NP_001044467 | 115440374 | Os01g0785400 [Oryza sativa Japonica Group] | 0.769706 | Oryza sativa Japonica Group | 3824 | 5107 |
| | | | | &gt:gi\|75272534\|sp\|Q8LQM5.1\|GH31_ORYSJ | | | | |
| | | | | RecName: Full = Probable indole-3-acetic | | | | |
| | | | | acid-amido synthetase GH3.1; | | | | |
| | | | | AltName: Full = Auxin-responsive GH3-like protein 1; | | | | |
| | | | | Short = OsGH3-1 | | | | |
| | | | | &gt:gi\|20804910\|dbj\|BAB92590.1\| | | | | |
| | | | | putative auxin-regulated protein GH3 | | | | |
| | | | | [Oryza sativa Japonica Group] | | | | |
| | | | | &gt:gi\|113533998\|dbj\|BAF06381.1\| | | | | |
| | | | | Os01g0785400 [Oryza sativa Japonica Group] | | | | |
| | | | | &gt:gi\|125572267\|gb\|EAZ13782.1\| | | | | |
| | | | | hypothetical protein OsJ_03707 | | | | |
| | | | | [Oryza sativa Japonica Group] | | | | |
| | | | | &gt:gi\|215693284\|dbj\|BAG88666.1\| | | | | |
| | | | | unnamed protein product | | | | |
| | | | | [Oryza sativa Japonica Group] | | | | |
| | | ACL52529 | 219884308 | unknown [Zea mays] | 0.734158 | Zea mays | 3825 | 5108 |
| | 489-508 | NP_001130602 | 212274530 | hypothetical protein LOC100191701 | 1 | Zea mays | 3826 | 5109 |
| | | | | [Zea mays] | | | | |
| | | | | &gt:gi\|194689604\|gb\|ACF78886.1\| | | | | |
| | | | | unknown [Zea mays] | | | | |
| | | | | &gt:gi\|219886741\|gb\|ACL53745.1\| | | | | |
| | | | | unknown [Zea mays] | | | | |
| | | XP_002457581 | 242056870 | hypothetical protein SORBIDRAFT_03g009790 | 0.868908 | Sorghum bicolor | 3827 | 5110 |
| | | | | [Sorghum bicolor] | | | | |
| | | | | &gt:gi\|241929556\|gb\|EES02701.1\| | | | | |
| | | | | hypothetical protein SORBIDRAFT_03g009790 | | | | |
| | | | | [Sorghum bicolor] | | | | |
| | | BAK07768 | 326522611 | predicted protein | 0.741176 | Hordeum vulgare subsp. vulgare | 3828 | 5111 |
| | | | | [Hordeum vulgare subsp. vulgare] | | | | |
| | | BAA84618 | 5922603 | putative pectinesterase | 0.712605 | Oryza sativa Japonica Group | 3829 | 5112 |
| | | | | [Oryza sativa Japonica Group] | | | | |
| | | | | &gt:gi\|6016850\|dbj\|BAA85193.1\| | | | | |
| | | | | putative pectinesterase | | | | |
| | | | | [Oryza sativa Japonica Group] | | | | |
| | 654-673 | XP_002458737 | 242059182 | hypothetical protein SORBIDRAFT_03g039330 | 1 | Sorghum bicolor | 3830 | 5113 |
| | | | | [Sorghum bicolor] | | | | |
| | | | | &gt:gi\|241930712\|gb\|EES03857.1\| | | | | |
| | | | | hypothetical protein SORBIDRAFT_03g039330 | | | | |
| | | | | [Sorghum bicolor] | | | | |
| | | NP_001147098 | 226508543 | osmotin-like protein [Zea mays] | 0.964143 | Zea mays | 3831 | 5114 |
| | | | | &gt:gi\|226958466\|ref\|NP_001152945.1\| | | | | |
| | | | | LOC100284104 [Zea mays] | | | | |
| | | | | &gt:gi\|195607196\|gb\|ACG25428.1\| | | | | |
| | | | | osmotin-like protein precursor [Zea mays] | | | | |
| | | | | &gt:gi\|195639504\|gb\|ACG39220.1\| | | | | |
| | | | | osmotin-like protein precursor [Zea mays] | | | | |
| | | ADI43217 | 297498988 | osmotin-like protein | 0888446 | Oryza sativa Indica Group | 3832 | 5115 |
| | | | | [Oryza sativa Indica Group] | | | | |
| | | NP_001044756 | 115440952 | Os01g0839900 [Oryza sativa Japonica Group] | 0.87251 | Oryza sativa Japonica Group | 3833 | 5116 |
| | | | | &gt:gi\|15623832\|dbj\|BAB67891.1\| | | | | |
| | | | | putative thaumatin-like cytokinin-binding | | | | |
| | | | | protein [Oryza sativa Japonica Group] | | | | |
| | | | | &gt:gi\|21104619\|dbj\|BAB93211.1\| | | | | |
| | | | | putative thaumatin-like cytokinin-binding protein | | | | |
| | | | | [Oryza sativa Japonica Group] | | | | |
| | | | | &gt:gi\|113534287\|dbj\|BAF06670.1\| | | | | |
| | | | | Os01g0839900 [Oryza sativa Japonica Group] | | | | |
| | | | | &gt:gi\|125528326\|gb\|EAY76440.1\| | | | | |
| | | | | hypothetical protein OsI_04374 | | | | |
| | | | | [Oryza sativa Indica Group] | | | | |
| | | | | &gt:gi\|125572584\|gb\|EAZ14099.1\| | | | | |
| | | | | hypothetical protein OsJ_04023 | | | | |
| | | | | [Oryza sativa Japonica Group] | | | | |
| | | | | &gt:gi\|215765831\|dbj\|BAG87528.1\| | | | | |
| | | | | unnamed protein product | | | | |
| | | | | [Oryza sativa Japonica Group] | | | | |
| | | ADI43216 | 297498986 | osmotin-like protein | 0.868526 | Oryza sativa Indica Group | 3834 | 5117 |
| | | | | [Oryza sativa Indica Group] | | | | |
| | | BAK00635 | 326524503 | predicted protein | 0.808765 | Hordeum vulgare subsp. vulgare | 3835 | 5118 |
| | | | | [Hordeum vulgare subsp. vulgare] | | | | |
| | 430-449 | ACN26514 | 223944860 | unknown [Zea mays] | 1 | Zea mays | 3836 | 5119 |
| | | NP_001151242 | 226533364 | triacylglycerol lipase [Zea mays] | 0.997512 | Zea mays | 3837 | 5120 |
| | | | | &gt:gi\|195645276\|gb\|ACG42106.1\| | | | | |
| | | | | triacylglycerol lipase [Zea mays] | | | | |
| | | XP_002441554 | 242091442 | hypothetical protein SORBIDRAFT_09g029230 | 0.81592 | Sorghum bicolor | 3838 | 5121 |
| | | | | [Sorghum bicolor] | | | | |
| | | | | &gt:gi\|241946839\|gb\|EES19984.1\| | | | | |
| | | | | hypothetical protein SORBIDRAFT_09g029230 | | | | |
| | | | | [Sorghum bicolor] | | | | |
| | | NP_001132361 | 212722431 | hypothetical protein LOC100193806 [Zea mays] | 0.716418 | Zea mays | 3839 | 5122 |
| | | | | &gt:gi\|194694184\|gb\|ACF81176.1\| | | | | |
| | | | | unknown [Zea mays] | | | | |
| | | XP_002440467 | 242089268 | hypothetical protein SORBIDRAFT_09g001420 | 0.706468 | Sorghum bicolor | 3840 | 5123 |
| | | | | [Sorghum bicolor] | | | | |
| | | | | &gt:gi\|241945752\|gb\|EES18897.1\| | | | | |
| | | | | hypothetical protein SORBIDRAFT_09g001420 | | | | |
| | | | | [Sorghum bicolor] | | | | |
| | | NP_001056387 | 115465574 | Os05g0574100 [Oryza sativa Japonica Group] | 0.803483 | Oryza sativa Japonica Group | 3841 | 5124 |
| | | | | &gt:gi\|75122568\|sp\|Q6F357.1\|PLA7_ORYSJ | | | | |
| | | | | RecName: Full = Phospholipase A1-II 7 | | | | |
| | | | | &gt:gi\|334350808\|sp\|A2Y7R2.1\|PLA7_ORYSI | | | | |
| | | | | RecName: Full = Phospholipase A1-II 7 | | | | |
| | | | | &gt:gi\|50080246\|gb\|AAT69581.1\| | | | | |
| | | | | putative lipase | | | | |
| | | | | [Oryza sativa Japonica Group] | | | | |
| | | | | &gt:gi\|52353545\|gb\|AAU44111.1\| | | | | |
| | | | | putative lipase | | | | |
| | | | | [Oryza sativa Japonica Group] | | | | |
| | | | | &gt:gi\|113579938\|dbj\|BAF18301.1\| | | | | |
| | | | | Os05g0574100 [Oryza sativa Japonica Group] | | | | |
| | | | | &gt:gi\|125553413\|gb\|EAY99122.1\| | | | | |
| | | | | hypothetical protein OsI_21081 | | | | |
| | | | | [Oryza sativa Indica Group] | | | | |
| | | XP_002465238 | 242035686 | hypothetical protein SORBIDRAFT_01g034810 | 0.708955 | Sorghum bicolor | 3842 | 5125 |
| | | | | [Sorghum bicolor] | | | | |
| | | | | &gt:gi\|241919092\|gb\|EER92236.1\| | | | | |
| | | | | hypothetical protein SORBIDRAFT_01g034810 | | | | |
| | | | | [Sorghum bicolor] | | | | |
| | 381-400 | NP_001152292 | 226492961 | LOC100285931 [Zea mays] | 1 | Zea mays | 3843 | 5126 |
| | | | | &gt:gi\|195654767\|gb\|ACG46851.1\| | | | | |
| | | | | poly [Zea mays] | | | | |
| | | | | &gt:gi\|238006566\|gb\|ACR34318.1\| | | | | |
| | | | | unknown [Zea mays] | | | | |
| | | XP_002465362 | 242035934 | hypothetical protein SORBIDRAFT_01g037200 | 0.891589 | Sorghum bicolor | 3844 | 5127 |
| | | | | [Sorghum bicolor] | | | | |
| | | | | &gt:gi\|241919216\|gb\|EER92360.1\| | | | | |
| | | | | hypothetical protein SORBIDRAFT_01g037200 | | | | |
| | | | | [Sorghum bicolor] | | | | |
| | 419-438 | XP_002455452 | 242052612 | hypothetical protein SORBIDRAFT_03g011010 | 1 | Sorghum bicolor | 3845 | 5128 |
| | | | | [Sorghum bicolor] | | | | |
| | | | | &gt:gi\|63087720\|emb\|CAI93175.1\| | | | | |
| | | | | glycosyltransferase [Sorghum bicolor] | | | | |
| | | | | &gt:gi\|241927427\|gb\|EES00572.1\| | | | | |
| | | | | hypothetical protein SORBIDRAFT_03g011010 | | | | |
| | | | | [Sorghum bicolor] | | | | |
| | 439-458 | NP_001141101 | 226505471 | hypothetical protein LOC100273184 | 1 | Zea mays | 3846 | 5129 |
| | | | | [Zea mays] | | | | |
| | | | | &gt:gi\|194702624\|gb\|ACF85396.1\| | | | | |
| | | | | unknown [Zea mays] | | | | |
| | | XP_002456304 | 242054316 | hypothetical protein SORBIDRAFT_03g033760 | 0.883772 | Sorghum bicolor | 3847 | 5130 |
| | | | | [Sorghum bicolor] | | | | |
| | | | | &gt:gi\|241928279\|gb\|EES01424.1\| | | | | |
| | | | | hypothetical protein SORBIDRAFT_03g033760 | | | | |
| | | | | [Sorghum bicolor] | | | | |
| | | NP_001044161 | 115439762 | Os01g0733500 [Oryza sativa Japonica Group] | 0.747807 | Oryza sativa Japonica Group | 3848 | 5131 |
| | | | | &gt:gi\|75164073\|sp\|Q942D4.1\|BURP3_ORYSJ | | | | |
| | | | | RecName: Full = BURP domain-containing protein 3; | | | | |
| | | | | Short = OsBURP03; Flags: Precursor | | | | |
| | | | | &gt:gi\|15624018\|dbj\|BAB68072.1\| | | | | |
| | | | | putative BURP domain-containing protein | | | | |
| | | | | [Oryza sativa Japonica Group] | | | | |
| | | | | &gt:gi\|20161002\|dbj\|BAB89935.1\| | | | | |
| | | | | putative BURP domain-containing protein | | | | |
| | | | | [Oryza sativa Japonica Group] | | | | |
| | | | | &gt:gi\|113533692\|dbj\|BAF06075.1\| | | | | |
| | | | | Os01g0733500 [Oryza sativa Japonica Group] | | | | |
| | | | | &gt:gi\|215694492\|dbj\|BAG89485.1\| | | | | |
| | | | | unnamed protein product | | | | |
| | | | | [Oryza sativa Japonica Group] | | | | |
| | | | | &gt:gi\|215741032\|dbj\|BAG97527.1\| | | | | |
| | | | | unnamed protein product | | | | |
| | | | | [Oryza sativa Japonica Group] | | | | |
| | | EAZ13440 | 54398660 | hypothetical protein OsJ_03360 | 0.745614 | Oryza sativa Japonica Group | 3849 | |
| | | | | [Oryza sativa Japonica Group] | | | | |
| | | EAY75726 | 54362548 | hypothetical protein OsI_03637 | 0.741228 | Oryza sativa Indica Group | 3850 | |
| | | | | [Oryza sativa Indica Group] | | | | |
| | | BAK06506 | 326502027 | predicted protein | 0.962719 | Hordeum vulgare subsp. vulgare | 3851 | 5132 |
| | | | | [Hordeum vulgare subsp. vulgare] | | | | |
| | 328-347 | ACN36219 | 224034286 | unknown [Zea mays] | 1 | Zea mays | 3852 | 5133 |
| | | NP_001150471 | 226532659 | LOC100284101 [Zea mays] | 0.780684 | Zea mays | 3853 | 5134 |
| | | | | &gt:gi\|195639480\|gb\|ACG39208.1\| | | | | |
| | | | | RNA-binding protein-like [Zea mays] | | | | |
| | 486-505 | XP_002464945 | 242035100 | hypothetical protein SORBIDRAFT_01g029270 | 1 | Sorghum bicolor | 3854 | 5135 |
| | | | | [Sorghum bicolor] | | | | |
| | | | | &gt:gi\|241918799\|gb\|EER91943.1\| | | | | |
| | | | | hypothetical protein SORBIDRAFT_01g029270 | | | | |
| | | | | [Sorghum bicolor] | | | | |
| | | NP_001105643 | 162458363 | beta-expansin 7 [Zea mays] | 0.924812 | Zea mays | 3855 | 5136 |
| | | | | &gt:gi\|14193773\|gb\|AAK56130.1\|AF332180_1 | | | | |
| | | | | beta-expansin 7 [Zea mays] | | | | |
| | | XP_002464944 | 242035098 | hypothetical protein SORBIDRAFT_01g029260 | 0.909774 | Sorghum bicolor | 3856 | 5137 |
| | | | | [Sorghum bicolor] | | | | |
| | | | | &gt:gi\|241918798\|gb\|EER91942.1\| | | | | |
| | | | | hypothetical protein SORBIDRAFT_01g029260 | | | | |
| | | | | [Sorghum bicolor] | | | | |
| | | NP_001148695 | 226494268 | beta-expansin 1a [Zea mays] | 0.887218 | Zea mays | 3857 | 5138 |
| | | | | &gt:gi\|195621464\|gb\|ACG32562.1\| | | | | |
| | | | | beta-expansin 1a precursor [Zea mays] | | | | |
| | | | | &gt:gi\|238010538\|gb\|ACR36304.1\| | | | | |
| | | | | unknown [Zea mays] | | | | |
| | | XP_002464943 | 242035096 | hypothetical protein SORBIDRAFT_01g029250 | 0.883459 | Sorghum bicolor | 3858 | 5139 |
| | | | | [Sorghum bicolor] | | | | |
| | | | | &gt:gi\|241918797\|gb\|EER91941.1\| | | | | |
| | | | | hypothetical protein SORBIDRAFT_01g029250 | | | | |
| | | | | [Sorghum bicolor] | | | | |
| | | XP_002464946 | 242035102 | hypothetical protein SORBIDRAFT_01g029273 | 0.868421 | Sorghum bicolor | 3859 | 5140 |
| | | | | [Sorghum bicolor] | | | | |
| | | | | &gt:gi\|241918800\|gb\|EER91944.1\| | | | | |
| | | | | hypothetical protein SORBIDRAFT_01g029273 | | | | |
| | | | | [Sorghum bicolor] | | | | |
| | | NP_001065351 | 115483361 | Os10g0555900 [Oryza sativa Japonica Group] | 0.834586 | Oryza sativa Japonica Group | 3860 | 5141 |
| | | | | &gt:gi\|115502197\|sp\|Q336T5.2\|EXPB3_ORYSJ | | | | |
| | | | | RecName: Full = Expansin-B3; | | | | |
| | | | | AltName: Full = Beta-expansin-3; | | | | |
| | | | | AltName: Full = OsEXPB3; | | | | |
| | | | | AltName: Full = OsaEXPb1.10; | | | | |
| | | | | Flags: Precursor | | | | |
| | | | | &gt:gi\|8118423\|gb\|AAF72984.1\|AF261271_2 | | | | |
| | | | | beta-expansin [Oryza sativa] | | | | |
| | | | | &gt:gi\|13194235\|gb\|AAK15453.1\|AC037426_15 | | | | |
| | | | | beta-expansin EXPB3 | | | | |
| | | | | [Oryza sativa Japonica Group] | | | | |
| | | | | &gt:gi\|14165333\|gb\|AAK55465.1\|AC069300_20 | | | | |
| | | | | beta-expansin (EXPB3) | | | | |
| | | | | [Oryza sativa Japonica Group] | | | | |
| | | | | &gt:gi\|110289548\|gb\|ABB47975.2\| | | | | |
| | | | | Beta-expansin 1a precursor, putative, | | | | |
| | | | | expressed [Oryza sativa Japonica Group] | | | | |
| | | | | &gt:gi\|113639883\|dbj\|BAF27188.1\| | | | | |
| | | | | Os10g0555900 [Oryza sativa Japonica Group] | | | | |
| | | | | &gt:gi\|125532914\|gb\|EAY79479.1\| | | | | |
| | | | | hypothetical protein OsI_34607 | | | | |
| | | | | [Oryza sativa Indica Group] | | | | |
| | | | | &gt:gi\|169244487\|gb\|ACA50517.1\| | | | | |
| | | | | beta-expansin [Oryza sativa Japonica Group] | | | | |
| | | | | &gt:gi\|215704824\|dbj\|BAG94852.1\| | | | | |
| | | | | unnamed protein product | | | | |
| | | | | [Oryza sativa Japonica Group] | | | | |
| | | XP_002452339 | 242062099 | hypothetical protein SORBIDRAFT_04g023980 | 0.808271 | Sorghum bicolor | 3861 | 5142 |
| | | | | [Sorghum bicolor] | | | | |
| | | | | &gt:gi\|241932170\|gb\|EES05315.1\| | | | | |
| | | | | hypothetical protein SORBIDRAFT_04g023980 | | | | |
| | | | | [Sorghum bicolor] | | | | |
| | | AAS48881 | 44894799 | expansin EXPB3 [Triticum aestivum] | 0.819549 | Triticum aestivum | 3862 | 5143 |
| | | AAT99293 | 51242710 | beta-expansin TaEXPB2 | 0.819549 | Triticum aestivum | 3863 | 5144 |
| | | | | [Triticum aestivum] | | | | |
| | 285-304 | ACF81426 | 194694683 | unknown [Zea mays] | 1 | Zea mays | 3864 | 5145 |
| | | | | &gt:gi\|223946585\|gb\|ACN27376.1\| | | | | |
| | | | | unknown [Zea mays] | | | | |
| | 71-90 | XP_002445056 | 242080574 | hypothetical protein SORBIDRAFT_07g003430 | 1 | Sorghum bicolor | 3865 | 5146 |
| | | | | [Sorghum bicolor] | | | | |
| | | | | &gt:gi\|241941406\|gb\|EES14551.1\| | | | | |
| | | | | hypothetical protein SORBIDRAFT_07g003430 | | | | |
| | | | | [Sorghum bicolor] | | | | |
| | | NP_001132060 | 212721279 | hypothetical protein LOC100193472 | 0.813309 | Zea mays | 3866 | 5147 |
| | | | | [Zea mays] | | | | |
| | | | | &gt:gi\|194693322\|gb\|ACF80745.1\| | | | | |
| | | | | unknown [Zea mays] | | | | |
| | 579-598 | XP_002439103 | 242086540 | hypothetical protein SORBIDRAFT_09g000540 | 1 | Sorghum bicolor | 3867 | 5148 |
| | | | | [Sorghum bicolor] | | | | |
| | | | | &gt:gi\|241944388\|gb\|EES17533.1\| | | | | |
| | | | | hypothetical protein SORBIDRAFT_09g000540 | | | | |
| | | | | [Sorghum bicolor] | | | | |
| | | NP_001151078 | 226529502 | ras-related protein Rab11C [Zea mays] | 0.954545 | Zea mays | 3868 | 5149 |
| | | | | &gt:gi\|226958337\|ref\|NP_001152941.1\| | | | | |
| | | | | ras-related protein Rab11C [Zea mays] | | | | |
| | | | | &gt:gi\|195629564\|gb\|ACG36423.1\| | | | | |
| | | | | ras-related protein Rab11C [Zea mays] | | | | |
| | | | | &gt:gi\|195644124\|gb\|ACG41530.1\| | | | | |
| | | | | ras-related protein Rab11C [Zea mays] | | | | |
| | | | | &gt:gi\|223973589\|gb\|ACN30982.1\| | | | | |
| | | | | unknown [Zea mays] | | | | |
| | | NP_001054408 | 115461616 | Os05g0105100 [Oryza sativa Japonica Group] | 0.945455 | Oryza sativa Japonica Group | 3869 | 5150 |
| | | | | &gt:gi\|46359908\|gb\|AAS88840.1\| | | | | |
| | | | | putative GTP-binding protein | | | | |
| | | | | [Oryza sativa Japonica Group] | | | | |
| | | | | &gt:gi\|52353601\|gb\|AAU44167.1\| | | | | |
| | | | | putative GTP-binding protein | | | | |
| | | | | [Oryza sativa Japonica Group] | | | | |
| | | | | &gt:gi\|113577959\|dbj\|BAF16322.1\| | | | | |
| | | | | Os05g0105100 [Oryza sativa Japonica Group] | | | | |
| | | | | &gt:gi\|125550513\|gb\|EAY96222.1\| | | | | |
| | | | | hypothetical protein OsI_18113 | | | | |
| | | | | [Oryza sativa Indica Group] | | | | |
| | | | | &gt:gi\|215734964\|dbj\|BAG95686.1\| | | | | |
| | | | | unnamed protein product | | | | |
| | | | | [Oryza sativa Japonica Group] | | | | |
| | | | | &gt:gi\|222629896\|gb\|EEE62028.1\| | | | | |
| | | | | hypothetical protein OsJ_16810 | | | | |
| | | | | [Oryza sativa Japonica Group] | | | | |
| | | BAK04054 | 326521851 | predicted protein | 0.859091 | Hordeum vulgare subsp. vulgare | 3870 | 5151 |
| | | | | [Hordeum vulgare subsp. vulgare] | | | | |
| | | XP_002892514 | 297849265 | hypothetical protein ARALYDRAFT_888208 | 0.781818 | Arabidopsis lyrata subsp. lyrata | 3871 | 5152 |
| | | | | [Arabidopsis lyrata subsp. lyrata] | | | | |
| | | | | &gt:gi\|297338356\|gb\|EFH68773.1\| | | | | |
| | | | | hypothetical protein ARALYDRAFT_888208 | | | | |
| | | | | [Arabidopsis lyrata subsp. lyrata] | | | | |
| | | NP_172434 | 30681250 | Ras-related protein RABA2a | 0.777273 | Arabidopsis thaliana | 3872 | 5153 |
| | | | | [Arabidopsis thaliana] | | | | |
| | | | | &gt:gi\|3024516\|sp\|O04486.1\|RAA2A_ARATH | | | | |
| | | | | RecName: Full = Ras-related protein RABA2a; | | | | |
| | | | | Short = AtRABA2a; | | | | |
| | | | | AltName: Full = Ras-related protein Rab11C; | | | | |
| | | | | Short = AtRab11C; Flags: Precursor | | | | |
| | | | | &gt:gi\|2160157\|gb\|AAB60720.1\| | | | | |
| | | | | Strong similarity to A. thaliana | | | | |
| | | | | ara-2 (gb\|ATHARA2). ESTs | | | | |
| | | | | gb\|ATTS2483, gb\|ATTS2484, gb\|AA042159 | | | | |
| | | | | come from this gene | | | | |
| | | | | [Arabidopsis thaliana] | | | | |
| | | | | &gt:gi\|2231303\|gb\|AAB61994.1\| | | | | |
| | | | | ras-related small GTPase | | | | |
| | | | | [Arabidopsis thaliana] | | | | |
| | | | | &gt:gi\|15010638\|gb\|AAK73978.1\| | | | | |
| | | | | At1g09630/F21M12_2 [Arabidopsis thaliana] | | | | |
| | | | | &gt:gi\|21553810\|gb\|AAM62903.1\| | | | | |
| | | | | putative RAS-related protein RAB11C | | | | |
| | | | | [Arabidopsis thaliana] | | | | |
| | | | | &gt:gi\|22137284\|gb\|AAM91487.1\| | | | | |
| | | | | At1g09630/F21M12_2 [Arabidopsis thaliana] | | | | |
| | | | | &gt:gi\|332190350\|gb\|AEE28471.1\| | | | | |
| | | | | Ras-related protein RABA2a | | | | |
| | | | | [Arabidopsis thaliana] | | | | |
| | | XP_002264444 | 225465199 | PREDICTED: hypothetical protein | 0.795455 | Vitis vinifera | 3873 | 5154 |
| | | | | [Vitis vinifera] | | | | |
| | | | | &gt:gi\|297739506\|emb\|CBI29688.3\| | | | | |
| | | | | unnamed protein product | | | | |
| | | | | [Vitis vinifera] | | | | |
| | | NP_001147252 | 226491887 | ras-related protein Rab11C [Zea mays] | 0.781818 | Zea mays | 3874 | 5155 |
| | | | | &gt:gi\|195609118\|gb\|ACG26389.1\| | | | | |
| | | | | ras-related protein Rab11C [Zea mays] | | | | |
| | | ACU23049 | 255645099 | unknown [Glycine max] | 0.772727 | Glycine max | 3875 | 5156 |
| | | XP_002331128 | 224120065 | predicted protein [Populus trichocarpa] | 0.772727 | Populus trichocarpa | 3876 | 5157 |
| | | | | &gt:gi\|222872856\|gb\|EEF09987.1\| | | | | |
| | | | | predicted protein [Populus trichocarpa] | | | | |
| | 627-646 | XP_002489255 | 253761759 | hypothetical protein SORBIDRAFT_0012s021070 | 1 | Sorghum bicolor | 3877 | 5158 |
| | | | | [Sorghum bicolor] | | | | |
| | | | | &gt:gi\|241947115\|gb\|EES20260.1\| | | | | |
| | | | | hypothetical protein SORBIDRAFT_0012s021070 | | | | |
| | | | | [Sorghum bicolor] | | | | |
| | | NP_001149604 | 226532613 | LOC100283230 [Zea mays] | 0.976959 | Zea mays | 3878 | 5159 |
| | | | | &gt:gi\|195628412\|gb\|ACG36036.1\| | | | | |
| | | | | ras-related protein Rab11D [Zea mays] | | | | |
| | | | | &gt:gi\|224032313\|gb\|ACN35232.1\| | | | | |
| | | | | unknown [Zea mays] | | | | |
| | | NP_001064476 | 115481765 | Os10g0377400 [Oryza sativa Japonica Group] | 0.953917 | Oryza sativa Japonica Group | 3879 | 5160 |
| | | | | &gt:gi\|78708412\|gb\|ABB47387.1\| | | | | |
| | | | | Ras-related protein Rab11D, putative, | | | | |
| | | | | expressed [Oryza sativa Japonica Group] | | | | |
| | | | | &gt:gi\|113639085\|dbj\|BAF26390.1\| | | | | |
| | | | | Os10g0377400 [Oryza sativa Japonica Group] | | | | |
| | | | | &gt:gi\|215767227\|dbj\|BAG99455.1\| | | | | |
| | | | | unnamed protein product | | | | |
| | | | | [Oryza sativa Japonica Group] | | | | |
| | | | | &gt:gi\|218184417\|gb\|EEC66844.1\| | | | | |
| | | | | hypothetical protein OsI_33317 | | | | |
| | | | | [Oryza sativa Indica Group] | | | | |
| | | | | &gt:gi\|222612729\|gb\|EEE50861.1\| | | | | |
| | | | | hypothetical protein OsJ_31310 | | | | |
| | | | | [Oryza sativa Japonica Group] | | | | |
| | | XP_002465727 | 242036664 | hypothetical protein SORBIDRAFT_01g044550 | 0.917051 | Sorghum bicolor | 3880 | 5161 |
| | | | | [Sorghum bicolor] | | | | |
| | | | | &gt:gi\|241919581\|gb\|EER92725.1\| | | | | |
| | | | | hypothetical protein SORBIDRAFT_01g044550 | | | | |
| | | | | [Sorghum bicolor] | | | | |
| | | NP_001151068 | 226529762 | ras-related protein Rab11D [Zea mays] | 0.917051 | Zea mays | 3881 | 5162 |
| | | | | &gt:gi\|226958362\|ref\|NP_001152898.1\| | | | | |
| | | | | hypothetical protein LOC100272235 | | | | |
| | | | | [Zea mays] | | | | |
| | | | | &gt:gi\|194696242\|gb\|ACF82205.1\| | | | | |
| | | | | unknown [Zea mays] | | | | |
| | | | | &gt:gi\|195644068\|gb\|ACG41502.1\| | | | | |
| | | | | ras-related protein Rab11D [Zea mays] | | | | |
| | | NP_001151560 | 226503740 | ras-related protein Rab11D [Zea mays] | 0.912442 | Zea mays | 3882 | 5163 |
| | | | | &gt:gi\|194708702\|gb\|ACF88435.1\| | | | | |
| | | | | unknown [Zea mays] | | | | |
| | | | | &gt:gi\|195647718\|gb\|ACG43327.1\| | | | | |
| | | | | ras-related protein Rab11D [Zea mays] | | | | |
| | | BAJ93377 | 326528454 | predicted protein | 0.921659 | Hordeum vulgare subsp. vulgare | 3883 | 5164 |
| | | | | [Hordeum vulgare subsp. vulgare] | | | | |
| | | AAM08543 | 17298574 | Putative Ras-related protein Rab | 0.889401 | Oryza sativa Japonica Group | 3884 | 5165 |
| | | | | [Oryza sativa Japonica Group] | | | | |
| | | XP_002310552 | 224096150 | predicted protein [Populus trichocarpa] | 0.843318 | Populus trichocarpa | 3885 | 5166 |
| | | | | &gt:gi\|222853455\|gb\|EEE91002.1\| | | | | |
| | | | | predicted protein [Populus trichocarpa] | | | | |
| | | XP_002273530 | 225468179 | PREDICTED: hypothetical protein | 0.829493 | Vitis vinifera | 3886 | 5167 |
| | | | | [Vitis vinifera] | | | | |
| | | | | &gt:gi\|147811153\|emb\|CAN70163.1\| | | | | |
| | | | | hypothetical protein VITISV_039255 | | | | |
| | | | | [Vitis vinifera] | | | | |
| | 241-260 | NP_001141352 | 226529852 | hypothetical protein LOC100273443 | 1 | Zea mays | 3887 | 5168 |
| | | | | [Zea mays] | | | | |
| | | | | &gt:gi\|194704134\|gb\|ACF86151.1\| | | | | |
| | | | | unknown [Zea mays] | | | | |
| | 902-921 | NP_001168477 | 293334238 | hypothetical protein LOC100382253 | 1 | Zea mays | 3888 | 5169 |
| | | | | [Zea mays] | | | | |
| | | | | &gt:gi\|223948517\|gb\|ACN28342.1\| | | | | |
| | | | | unknown [Zea mays] | | | | |
| | | ACR36409 | 238010747 | unknown [Zea mays] | 0.870253 | Zea mays | 3889 | 5170 |
| | 615-634 | XP_002437204 | 242093427 | hypothetical protein SORBIDRAFT_10g022830 | 1 | Sorghum bicolor | 3890 | 5171 |
| | | | | [Sorghum bicolor] | | | | |
| | | | | &gt:gi\|241915427\|gb\|EER88571.1\| | | | | |
| | | | | hypothetical protein SORBIDRAFT_10g022830 | | | | |
| | | | | [Sorghum bicolor] | | | | |
| | | NP_001150723 | 226503328 | pectate lyase [Zea mays] | 0.865639 | Zea mays | 3891 | 5172 |
| | | | | &gt:gi\|195641318\|gb\|ACG40127.1\| | | | | |
| | | | | pectate lyase precursor [Zea mays] | | | | |
| | | NP_001057945 | 115468691 | Os06g0583900 [Oryza sativa Japonica Group] | 0.751101 | Oryza sativa Japonica Group | 3892 | 5173 |
| | | | | &gt:gi\|55296520\|dbj\|BAD68734.1\| | | | | |
| | | | | putative pectate lyase | | | | |
| | | | | [Oryza sativa Japonica Group] | | | | |
| | | | | &gt:gi\|113595985\|dbj\|BAF19859.1\| | | | | |
| | | | | Os06g0583900 [Oryza sativa Japonica Group] | | | | |
| | | | | &gt:gi\|215704206\|dbj\|BAG93046.1\| | | | | |
| | | | | unnamed protein product | | | | |
| | | | | [Oryza sativa Japonica Group] | | | | |
| | | | | &gt:gi\|218198444\|gb\|EEC80871.1\| | | | | |
| | | | | hypothetical protein OsI_23497 | | | | |
| | | | | [Oryza sativa Indica Group] | | | | |
| | | BAK06965 | 326513449 | predicted protein | 0.729075 | Hordeum vulgare subsp. vulgare | 3893 | 5174 |
| | | | | [Hordeum vulgare subsp. vulgare] | | | | |
| | 329-348 | XP_002447622 | 242075371 | hypothetical protein SORBIDRAFT_06g009000 | 1 | Sorghum bicolor | 3894 | 5175 |
| | | | | [Sorghum bicolor] | | | | |
| | | | | &gt:gi\|241938805\|gb\|EES11950.1\| | | | | |
| | | | | hypothetical protein SORBIDRAFT_06g009000 | | | | |
| | | | | [Sorghum bicolor] | | | | |
| | 759-778 | NP_001130136 | 212275801 | hypothetical protein LOC100191230 | 1 | Zea mays | 3895 | 5176 |
| | | | | [Zea mays] | | | | |
| | | | | &gt:gi\|194688374\|gb\|ACF78271.1\| | | | | |
| | | | | unknown [Zea mays] | | | | |
| | | | | &gt:gi\|223946699\|gb\|ACN27433.1\| | | | | |
| | | | | unknown [Zea mays] | | | | |
| | | | | &gt:gi\|224033665\|gb\|ACN35908.1\| | | | | |
| | | | | unknown [Zea mays] | | | | |
| | | ACG32118 | 195620575 | exopolygalacturonase precursor | 0.985577 | Zea mays | 3896 | 5177 |
| | | | | [Zea mays] | | | | |
| | | XP_002455693 | 242053094 | hypothetical protein SORBIDRAFT_03g021050 | 0.834135 | Sorghum bicolor | 3897 | 5178 |
| | | | | [Sorghum bicolor] | | | | |
| | | | | &gt:gi\|241927668\|gb\|EES00813.1\| | | | | |
| | | | | hypothetical protein SORBIDRAFT_03g021050 | | | | |
| | | | | [Sorghum bicolor] | | | | |
| | | NP_001141594 | 226503987 | hypothetical protein LOC100273711 | 0.831731 | Zea mays | 3898 | 5179 |
| | | | | [Zea mays] | | | | |
| | | | | &gt:gi\|194705208\|gb\|ACF86688.1\| | | | | |
| | | | | unknown [Zea mays] | | | | |
| | | ACG34252 | 195624843 | exopolygalacturonase precursor | 0.829327 | Zea mays | 3899 | 5180 |
| | | | | [Zea mays] | | | | |
| | 419-438 | XP_002464517 | 242034244 | hypothetical protein SORBIDRAFT_01g019940 | 1 | Sorghum bicolor | 3900 | 5181 |
| | | | | [Sorghum bicolor] | | | | |
| | | | | &gt:gi\|241918371\|gb\|EER91515.1\| | | | | |
| | | | | hypothetical protein SORBIDRAFT_01g019940 | | | | |
| | | | | [Sorghum bicolor] | | | | |
| | | NP_001168725 | 293336668 | hypothetical protein LOC100382517 | 0.858 | Zea mays | 3901 | 5182 |
| | | | | [Zea mays] | | | | |
| | | | | &gt:gi\|223950415\|gb\|ACN29291.1\| | | | | |
| | | | | unknown [Zea mays] | | | | |
| | 1035-1054 | XP_002447810 | 242075747 | hypothetical protein SORBIDRAFT_06g016230 | 1 | Sorghum bicolor | 3902 | 5183 |
| | | | | [Sorghum bicolor] | | | | |
| | | | | &gt:gi\|241938993\|gb\|EES12138.1\| | | | | |
| | | | | hypothetical protein SORBIDRAFT_06g016230 | | | | |
| | | | | [Sorghum bicolor] | | | | |
| | 654-673 | XP_002460936 | 242046129 | hypothetical protein SORBIDRAFT_02g037770 | 1 | Sorghum bicolor | 3903 | 5184 |
| | | | | [Sorghum bicolor] | | | | |
| | | | | &gt:gi\|241924313\|gb\|EER97457.1\| | | | | |
| | | | | hypothetical protein SORBIDRAFT_02g037770 | | | | |
| | | | | [Sorghum bicolor] | | | | |
| | | NP_001145615 | 226507742 | hypothetical protein LOC100279098 | 0.873846 | Zea mays | 3904 | 5185 |
| | | | | [Zea mays] | | | | |
| | | | | &gt:gi\|195658887\|gb\|ACG48911.1\| | | | | |
| | | | | hypothetical protein [Zea mays] | | | | |
| | | NP_001145067 | 226495966 | hypothetical protein LOC100278263 | 0.830769 | Zea mays | 3905 | 5186 |
| | | | | [Zea mays] | | | | |
| | | | | &gt:gi\|195650593\|gb\|ACG44764.1\| | | | | |
| | | | | hypothetical protein [Zea mays] | | | | |
| | 1012-1031 | XP_002452495 | 242062411 | hypothetical protein SORBIDRAFT_04g026900 | 1 | Sorghum bicolor | 3906 | 5187 |
| | | | | [Sorghum bicolor] | | | | |
| | | | | &gt:gi\|241932326\|gb\|EES05471.1\| | | | | |
| | | | | hypothetical protein SORBIDRAFT_04g026900 | | | | |
| | | | | [Sorghum bicolor] | | | | |
| | | ACF88069 | 194707969 | unknown [Zea mays] | 0.898947 | Zea mays | 3907 | 5188 |
| | | NP_001152245 | 226533273 | lysosomal protective protein [Zea mays] | 0.907368 | Zea mays | 3908 | 5189 |
| | | | | &gt:gi\|195654245\|gb\|ACG46590.1\| | | | | |
| | | | | lysosomal protective protein precursor | | | | |
| | | | | [Zea mays] | | | | |
| | | NP_001047514 | 115447468 | Os02g0634700 [Oryza sativa Japonica Group] | 0.825263 | Oryza sativa Japonica Group | 3909 | 5190 |
| | | | | &gt:gi\|49387538\|dbj\|BAD25094.1\| | | | | |
| | | | | putative carboxypeptidase D | | | | |
| | | | | [Oryza sativa Japonica Group] | | | | |
| | | | | &gt:gi\|49388186\|dbj\|BAD25312.1\| | | | | |
| | | | | putative carboxypeptidase D | | | | |
| | | | | [Oryza sativa Japonica Group] | | | | |
| | | | | &gt:gi\|113537045\|dbj\|BAF09428.1\| | | | | |
| | | | | Os02g0634700 [Oryza sativa Japonica Group] | | | | |
| | | | | &gt:gi\|215737473\|dbj\|BAG96603.1\| | | | | |
| | | | | unnamed protein product | | | | |
| | | | | [Oryza sativa Japonica Group] | | | | |
| | | | | &gt:gi\|215741081\|dbj\|BAG97576.1\| | | | | |
| | | | | unnamed protein product | | | | |
| | | | | [Oryza sativa Japonica Group] | | | | |
| | | | | &gt:gi\|222623302\|gb\|EEE57434.1\| | | | | |
| | | | | hypothetical protein OsJ_07638 | | | | |
| | | | | [Oryza sativa Japonica Group] | | | | |
| | | EEC73659 | 54362548 | hypothetical protein OsI_08191 | 0.825263 | Oryza sativa Indica Group | 3910 | |
| | | | | [Oryza sativa Indica Group] | | | | |
| | | ABF70080 | 102139931 | serine carboxypeptidase (carboxypeptidase D), | 0.722105 | Musa acuminata | 3911 | 5191 |
| | | | | putative [Musa acuminata] | | | | |
| | 1137-1156 | XP_002451244 | 242071934 | hypothetical protein SORBIDRAFT_05g026400 | 1 | Sorghum bicolor | 3912 | 5192 |
| | | | | [Sorghum bicolor] | | | | |
| | | | | &gt:gi\|241937087\|gb\|EES10232.1\| | | | | |
| | | | | hypothetical protein SORBIDRAFT_05g026400 | | | | |
| | | | | [Sorghum bicolor] | | | | |
| | | NP_001145980 | 226531268 | hypothetical protein LOC100279508 | 0.834177 | Zea mays | 3913 | 5193 |
| | | | | [Zea mays] | | | | |
| | | | | &gt:gi\|219885199\|gb\|ACL52974.1\| | | | | |
| | | | | unknown [Zea mays] | | | | |
| | | NP_001068441 | 115486594 | Os11g0673200 [Oryza sativa Japonica Group] | 0.777215 | Oryza sativa Japonica Group | 3914 | 5194 |
| | | | | &gt:gi\|113645663\|dbj\|BAF28804.1\| | | | | |
| | | | | Os11g0673200 [Oryza sativa Japonica Group] | | | | |
| | | ACG29242 | 195614823 | auxin-induced beta-glucosidase [Zea mays] | 0.702532 | Zea mays | 3915 | 5195 |
| | | EAY81823 | 54362548 | hypothetical protein OsI_36995 | 0.775949 | Oryza sativa Indica Group | 3916 | |
| | | | | [Oryza sativa Indica Group] | | | | |
| | | ABA95273 | 108863896 | Beta-D-xylosidase, putative, expressed | 0.777215 | Oryza sativa Japonica Group | 3917 | |
| | | | | [Oryza sativa Japonica Group] | | | | |
| | 951-970 | NP_001147443 | 226493965 | peroxidase 52[Zea mays] | 1 | Zea mays | 3918 | 5196 |
| | | | | &gt:gi\|195611432\|gb\|ACG27546.1\| | | | | |
| | | | | peroxidase 52 precursor [Zea mays] | | | | |
| | | EEC75221 | 54362548 | hypothetical protein OsI_11488 | 0.779456 | Oryza sativa Indica Group | 3919 | |
| | | | | [Oryza sativa Indica Group] | | | | |
| | | CAH69282 | 55700946 | TPA: class III peroxidase 40 precursor | 0.776435 | Oryza sativa Japonica Group | 3920 | 5197 |
| | | | | [Oryza sativa Japonica Group] | | | | |
| | | ABF95843 | 108705663 | Peroxidase 52 precursor, putative, | 0.776435 | Oryza sativa Japonica Group | 3921 | |
| | | | | expressed [Oryza sativa Japonica Group] | | | | |
| | | | | &gt:gi\|222624896\|gb\|EEE59028.1\| | | | | |
| | | | | hypothetical protein OsJ_10775 | | | | |
| | | | | [Oryza sativa Japonica Group] | | | | |
| | | BAK00939 | 326529090 | predicted protein | 0.767372 | Hordeum vulgare subsp. vulgare | 3922 | 5198 |
| | | | | [Hordeum vulgare subsp. vulgare] | | | | |
| | 1197-1216 | ACG45528 | 195652120 | ubiquitin-protein ligase [Zea mays] | 1 | Zea mays | 3923 | 5199 |
| | | NP_001152043 | 226491951 | ubiquitin-protein ligase [Zea mays] | 0.997015 | Zea mays | 3924 | 5200 |
| | | | | &gt:gi\|194697400\|gb\|ACF82784.1\| | | | | |
| | | | | unknown [Zea mays] | | | | |
| | | | | &gt:gi\|219886369\|gb\|ACL53559.1\| | | | | |
| | | | | unknown [Zea mays] | | | | |
| | | | | &gt:gi\|223942179\|gb\|ACN25173.1\| | | | | |
| | | | | unknown [Zea mays] | | | | |
| | | | | &gt:gi\|224028595\|gb\|ACN33373.1\| | | | | |
| | | | | unknown [Zea mays] | | | | |
| | | XP_002454240 | 242065901 | hypothetical protein SORBIDRAFT_04g027350 | 0.877612 | Sorghum bicolor | 3925 | 5201 |
| | | | | [Sorghum bicolor] | | | | |
| | | | | &gt:gi\|241934071\|gb\|EES07216.1\| | | | | |
| | | | | hypothetical protein SORBIDRAFT_04g027350 | | | | |
| | | | | [Sorghum bicolor] | | | | |
| | | NP_001048172 | 115448784 | Os02g0757700 [Oryza sativa Japonica Group] | 0.79403 | Oryza sativa Japonica Group | 3926 | 5202 |
| | | | | &gt:gi\|46805687\|dbj\|BAD17088.1\| | | | | |
| | | | | F-box protein-like | | | | |
| | | | | [Oryza sativa Japonica Group] | | | | |
| | | | | &gt:gi\|113537703\|dbj\|BAF10086.1\| | | | | |
| | | | | Os02g0757700 [Oryza sativa Japonica Group] | | | | |
| | | | | &gt:gi\|125541199\|gb\|EAY87594.1\| | | | | |
| | | | | hypothetical protein OsI_09005 | | | | |
| | | | | [Oryza sativa Indica Group] | | | | |
| | | | | &gt:gi\|125583751\|gb\|EAZ24682.1\| | | | | |
| | | | | hypothetical protein OsJ_08452 | | | | |
| | | | | [Oryza sativa Japonica Group] | | | | |
| | | NP_001141525 | 226506077 | hypothetical protein LOC100273637 | 0.847761 | Zea mays | 3927 | 5203 |
| | | | | [Zea mays] | | | | |
| | | | | &gt:gi\|194696402\|gb\|ACF82285.1\| | | | | |
| | | | | unknown [Zea mays] | | | | |
| | | | | &gt:gi\|194704930\|gb\|ACF86549.1\| | | | | |
| | | | | unknown [Zea mays] | | | | |
| | | ACF79744 | 194691319 | unknown [Zea mays] | 0.844776 | Zea mays | 3928 | 5204 |
| | | ACG31703 | 195619745 | ubiquitin-protein ligase [Zea mays] | 0.844776 | Zea mays | 3929 | 5205 |
| | | NP_001057164 | 115467129 | Os06g0219700 [Oryza sativa Japonica Group] | 0.728358 | Oryza sativa Japonica Group | 3930 | 5206 |
| | | | | &gt:gi\|51535368\|dbj\|BAD37239.1\| | | | | |
| | | | | F-box protein-like | | | | |
| | | | | [Oryza sativa Japonica Group] | | | | |
| | | | | &gt:gi\|113595204\|dbj\|BAF19078.1\| | | | | |
| | | | | Os06g0219700 [Oryza sativa Japonica Group] | | | | |
| | | | | &gt:gi\|125554574\|gb\|EAZ00180.1\| | | | | |
| | | | | hypothetical protein OsI_22185 | | | | |
| | | | | [Oryza sativa Indica Group] | | | | |
| | | | | &gt:gi\|125596515\|gb\|EAZ36295.1\| | | | | |
| | | | | hypothetical protein OsJ_20616 | | | | |
| | | | | [Oryza sativa Japonica Group] | | | | |
| | | | | &gt:gi\|215697729\|dbj\|BAG91723.1\| | | | | |
| | | | | unnamed protein product | | | | |
| | | | | [Oryza sativa Japonica Group] | | | | |
| | | BAJ92240 | 326514179 | predicted protein | 0.701493 | Hordeum vulgare subsp. vulgare | 3931 | 5207 |
| | | | | [Hordeum vulgare subsp. vulgare] | | | | |
| | 779-798 | AAR30916 | 39777292 | phytochrome B [Sorghum propinquum] | 1 | Sorghum propinquum | 3932 | |
| | | AAR30915 | 39777290 | phytochrome B | 0.998301 | Sorghum bicolor subsp. × drummondii | 3933 | |
| | | | | [Sorghum bicolor subsp. × drummondii] | | | | |
| | | XP_002467973 | 242041156 | hypothetical protein SORBIDRAFT_01g037340 | 0.998301 | Sorghum bicolor | 3934 | 5208 |
| | | | | [Sorghum bicolor] | | | | |
| | | | | &gt:gi\|39777261\|gb\|AAR30900.1\| | | | | |
| | | | | phytochrome B [Sorghum bicolor] | | | | |
| | | | | &gt:gi\|39777263\|gb\|AAR30901.1\| | | | | |
| | | | | phytochrome B [Sorghum bicolor] | | | | |
| | | | | &gt:gi\|39777265\|gb\|AAR30902.1\| | | | | |
| | | | | phytochrome B [Sorghum bicolor] | | | | |
| | | | | &gt:gi\|39777269\|gb\|AAR30904.1\| | | | | |
| | | | | phytochrome B [Sorghum bicolor] | | | | |
| | | | | &gt:gi\|39777275\|gb\|AAR30907.1\| | | | | |
| | | | | phytochrome B | | | | |
| | | | | [Sorghum bicolor subsp. | | | | |
| | | | | verticilliflorum] | | | | |
| | | | | &gt:gi\|39777277\|gb\|AAR30908.1\| | | | | |
| | | | | phytochrome B | | | | |
| | | | | [Sorghum bicolor subsp. | | | | |
| | | | | verticilliflorum] | | | | |
| | | | | &gt:gi\|39777279\|gb\|AAR30909.1\| | | | | |
| | | | | phytochrome B | | | | |
| | | | | [Sorghum bicolor subsp. | | | | |
| | | | | verticilliflorum] | | | | |
| | | | | &gt:gi\|39777281\|gb\|AAR30910.1\| | | | | |
| | | | | phytochrome B | | | | |
| | | | | [Sorghum bicolor subsp. | | | | |
| | | | | verticilliflorum] | | | | |
| | | | | &gt:gi\|39777283\|gb\|AAR30911.1\| | | | | |
| | | | | phytochrome B | | | | |
| | | | | [Sorghum bicolor subsp. | | | | |
| | | | | verticilliflorum] | | | | |
| | | | | &gt:gi\|39777285\|gb\|AAR30912.1\| | | | | |
| | | | | phytochrome B | | | | |
| | | | | [Sorghum bicolor subsp. | | | | |
| | | | | verticilliflorum] | | | | |
| | | | | &gt:gi\|39777287\|gb\|AAR30913.1\| | | | | |
| | | | | phytochrome B | | | | |
| | | | | [Sorghum bicolor subsp. | | | | |
| | | | | verticilliflorum] | | | | |
| | | | | &gt:gi\|241921827\|gb\|EER94971.1\| | | | | |
| | | | | hypothetical protein SORBIDRAFT_01g037340 | | | | |
| | | | | [Sorghum bicolor] | | | | |
| | | AAR30914 | 39777288 | phytochrome B | 0.997451 | Sorghum bicolor subsp. | 3935 | |
| | | | | [Sorghum bicolor subsp. | | verticilliflorum | | |
| | | | | verticilliflorum] | | | | |
| | | AAR30903 | 39777266 | phytochrome B [Sorghum bicolor] | 0.997451 | Sorghum bicolor | 3936 | |
| | | AAR30905 | 39777270 | phytochrome B [Sorghum bicolor] | 0.996602 | Sorghum bicolor | 3937 | |
| | | | | &gt:gi\|39777273\|gb\|AAR30906.1\| | | | | |
| | | | | phytochrome B [Sorghum bicolor] | | | | |
| | | P93527 | | RecName: Full = Phytochrome B | 0.995752 | Sorghum bicolor | 3938 | |
| | | | | &gt:gi\|7110162\|gb\|AAB41398.2\| | | | | |
| | | | | phytochrome B [Sorghum bicolor] | | | | |
| | | NP_001169810 | 293335472 | hypothetical protein LOC100383702 | 0.95582 | Zea mays | 3939 | 5209 |
| | | | | [Zea mays] | | | | |
| | | | | &gt:gi\|37926866\|gb\|AAP06788.1\| | | | | |
| | | | | phytochrome B1 apoprotein [Zea mays] | | | | |
| | | NP_001168077 | 293336622 | hypothetical protein LOC100381811 | 0.932031 | Zea mays | 3940 | 5210 |
| | | | | [Zea mays] | | | | |
| | | | | &gt:gi\|37926881\|gb\|AAP06789.1\| | | | | |
| | | | | phytochrome B2 apoprotein [Zea mays] | | | | |
| | | Q10MG9 | | RecName: Full = Phytochrome B | 0.912489 | Oryza sativa Japonica Group | 3941 | |
| | | | | &gt:gi\|30578178\|dbj\|BAC76432.1\| | | | | |
| | | | | phytochrome B | | | | |
| | | | | [Oryza sativa Japonica Group] | | | | |
| | | | | &gt:gi\|57506674\|dbj\|BAD86669.1\| | | | | |
| | | | | phytochrome B | | | | |
| | | | | [Oryza sativa Japonica Group] | | | | |
| | | | | &gt:gi\|108707763\|gb\|ABF95558.1\| | | | | |
| | | | | Phytochrome B, putative, expressed | | | | |
| | | | | [Oryza sativa Japonica Group] | | | | |
| | 1398-1417 | NP_001146696 | 226503338 | hypothetical protein LOC100280297 | 1 | Zea mays | 3942 | 5211 |
| | | | | [Zea mays] | | | | |
| | | | | &gt:gi\|219888377\|gb\|ACL54563.1\| | | | | |
| | | | | unknown [Zea mays] | | | | |
| | 606-625 | NP_001151271 | 226500783 | beta-amylase [Zea mays] | 1 | Zea mays | 3943 | 5212 |
| | | | | &gt:gi\|195645426\|gb\|ACG42181.1\| | | | | |
| | | | | beta-amylase [Zea mays] | | | | |
| | | XP_002467860 | 242040930 | hypothetical protein SORBIDRAFT_01g035370 | 0.815642 | Sorghum bicolor | 3944 | 5213 |
| | | | | [Sorghum bicolor] | | | | |
| | | | | &gt:gi\|241921714\|gb\|EER94858.1\| | | | | |
| | | | | hypothetical protein SORBIDRAFT_01g035370 | | | | |
| | | | | [Sorghum bicolor] | | | | |
| Predicted | 269-290 | ACN35534 | 224032916 | unknown [Zea mays] | 1 | Zea mays | 3945 | 5214 |
| zma | | | | | | | | |
| mir | | | | | | | | |
| 50486 | | | | | | | | |
| | | ACG33974 | 195624287 | hypothetical protein [Zea mays] | 0.991189 | Zea mays | 3946 | 5215 |
| | | NP_001136584 | 219363442 | hypothetical protein LOC100216707 | 0.894273 | Zea mays | 3947 | 5216 |
| | | | | [Zea mays] | | | | |
| | | | | &gt:gi\|194696272\|gb\|ACF82220.1\| | | | | |
| | | | | unknown [Zea mays] | | | | |
| | | XP_002466626 | 242038462 | hypothetical protein SORBIDRAFT_01g011230 | 0.854626 | Sorghum bicolor | 3948 | 5217 |
| | | | | [Sorghum bicolor] | | | | |
| | | | | &gt:gi\|241920480\|gb\|EER93624.1\| | | | | |
| | | | | hypothetical protein SORBIDRAFT_01g011230 | | | | |
| | | | | [Sorghum bicolor] | | | | |
| | | NP_001050978 | 115454754 | Os03g0696000 [Oryza sativa Japonica Group] | 0.76652 | Oryza sativa Japonica Group | 3949 | 5218 |
| | | | | &gt:gi\|108710552\|gb\|ABF98347.1\| expressed | | | | |
| | | | | protein [Oryza sativa Japonica Group] | | | | |
| | | | | &gt:gi\|113549449\|dbj\|BAF12892.1\| | | | | |
| | | | | Os03g0696000 [Oryza sativa Japonica Group] | | | | |
| | | | | &gt:gi\|215766532\|dbj\|BAG98840.1\| | | | | |
| | | | | unnamed protein product | | | | |
| | | | | [Oryza sativa Japonica Group] | | | | |
| Predicted | 1103-1122 | NP_001130769 | 212276197 | hypothetical protein LOC100191873 [Zea mays] | 1 | Zea mays | 3950 | 5219 |
| zma | | | | &gt:gi\|194690070\|gb\|ACF79119.1\| | | | | |
| mir | | | | unknown [Zea mays] | | | | |
| 50570 | | | | | | | | |
| | | ACG47274 | 195655612 | cys2/His2 zinc-finger transcription | 0.961877 | Zea mays | 3951 | 5220 |
| | | | | factor [Zea mays] | | | | |
| | | NP_001152162 | 226532358 | cys2/His2 zinc-finger transcription | 0.777126 | Zea mays | 3952 | 5221 |
| | | | | factor [Zea mays] | | | | |
| | | | | &gt:gi\|195653369\|gb\|ACG46152.1\| | | | | |
| | | | | cys2/His2 zinc-finger transcription | | | | |
| | | | | factor [Zea mays] | | | | |
| | | | | &gt:gi\|223947915\|gb\|ACN28041.1\| | | | | |
| | | | | unknown [Zea mays] | | | | |
| | | XP_002464952 | 242035114 | hypothetical protein SORBIDRAFT_01g029370 | 0.765396 | Sorghum bicolor | 3953 | 5222 |
| | | | | [Sorghum bicolor] | | | | |
| | | | | &gt:gi\|241918806\|gb\|EER91950.1\| | | | | |
| | | | | hypothetical protein SORBIDRAFT_01g029370 | | | | |
| | | | | [Sorghum bicolor] | | | | |
| Predicted | 336-355 | ACG38830 | 195638723 | nucleotide binding protein [Zea mays] | 1 | Zea mays | 3954 | 5223 |
| zma | | | | | | | | |
| mir | | | | | | | | |
| 50682 | | | | | | | | |
| | | NP_001150233 | 226495198 | nucleotide binding protein [Zea mays] | 0.925466 | Zea mays | 3955 | 5224 |
| | | | | &gt:gi\|195637698\|gb\|ACG38317.1\| | | | | |
| | | | | nucleotide binding protein [Zea mays] | | | | |
| | 138-157 | XP_002453342 | 242064105 | hypothetical protein SORBIDRAFT_04g004 160 | 1 | Sorghum bicolor | 3956 | 5225 |
| | | | | [Sorghum bicolor] | | | | |
| | | | | &gt:gi\|241933173\|gb\|EES06318.1\| | | | | |
| | | | | hypothetical protein SORBIDRAFT_04g004 160 | | | | |
| | | | | [Sorghum bicolor] | | | | |
| | | EEE56349 | 54398660 | hypothetical protein OsJ_05464 | 0.838996 | Oryza sativa Japonica Group | 3957 | |
| | | | | [Oryza sativa Japonica Group] | | | | |
| | | BAD28046 | 45382008 | putative Helicase SKI2W | 0.774742 | Oryza sativa Japonica Group | 3958 | |
| | | | | [Oryza sativa Japonica Group] | | | | |
| | 554-573 | XP_002466420 | 242038050 | hypothetical protein SORBIDRAFT_01g007510 | 1 | Sorghum bicolor | 3959 | 5226 |
| | | | | [Sorghum bicolor] | | | | |
| | | | | &gt:gi\|241920274\|gb\|EER93418.1\| | | | | |
| | | | | hypothetical protein SORBIDRAFT_01g007510 | | | | |
| | | | | [Sorghum bicolor] | | | | |
| Predicted | 24-42 | XP_002437845 | 242094709 | hypothetical protein SORBIDRAFT_10g003610 | 1 | Sorghum bicolor | 3960 | 5227 |
| zma | | | | [Sorghum bicolor] | | | | |
| mir | | | | &gt:gi\|241916068\|gb\|EER89212.1\| | | | | |
| 50701 | | | | hypothetical protein SORBIDRAFT_10g003610 | | | | |
| | | | | [Sorghum bicolor] | | | | |
| | | NP_001149507 | 226500789 | LOC100283133 [Zea mays] | 0.986014 | Zea mays | 3961 | 5228 |
| | | | | &gt:gi\|195621266\|gb\|ACG32463.1\| | | | | |
| | | | | trafficking protein particle complex | | | | |
| | | | | subunit 4 [Zea mays] | | | | |
| | | | | &gt:gi\|195627666\|gb\|ACG35663.1\| | | | | |
| | | | | trafficking protein particle complex | | | | |
| | | | | subunit 4 [Zea mays] | | | | |
| | | | | &gt:gi\|223974417\|gb\|ACN31396.1\| | | | | |
| | | | | unknown [Zea mays] | | | | |
| | | | | &gt:gi\|223974509\|gb\|ACN3 1442.1\| | | | | |
| | | | | unknown [Zea mays] | | | | |
| | | NP_001131958 | 212721385 | hypothetical protein LOC100193354 | 0.979021 | Zea mays | 3962 | 5229 |
| | | | | [Zea mays] | | | | |
| | | | | &gt:gi\|194693036\|gb\|ACF80602.1\| | | | | |
| | | | | unknown [Zea mays] | | | | |
| | | | | &gt:gi\|194696020\|gb\|ACF82094.1\| | | | | |
| | | | | unknown [Zea mays] | | | | |
| | | | | &gt:gi\|223973055\|gb\|ACN30715.1\| | | | | |
| | | | | unknown [Zea mays] | | | | |
| | | ACG45824 | 195652712 | trafficking protein particle complex | 0.972028 | Zea mays | 3963 | 5230 |
| | | | | subunit 4 [Zea mays] | | | | |
| | | ACG47442 | 195655948 | trafficking protein particle complex | 0.972028 | Zea mays | 3964 | 5231 |
| | | | | subunit 4 [Zea mays] | | | | |
| | | NP_001059557 | 115471916 | Os07g0454700 [Oryza sativa Japonica Group] | 0.937063 | Oryza sativa Japonica Group | 3965 | 5232 |
| | | | | &gt:gi\|28971996\|dbj\|BAC65404.1\| | | | | |
| | | | | synbindin-like protein | | | | |
| | | | | [Oryza sativa Japonica Group] | | | | |
| | | | | &gt:gi\|113611093\|dbj\|BAF21471.1\| | | | | |
| | | | | Os07g0454700 [Oryza sativa Japonica Group] | | | | |
| | | | | &gt:gi\|125600106\|gb\|EAZ39682.1\| | | | | |
| | | | | hypothetical protein OsJ_24117 | | | | |
| | | | | [Oryza sativa Japonica Group] | | | | |
| | | | | &gt:gi\|215764944\|dbj\|BAG86641.1\| | | | | |
| | | | | unnamed protein product | | | | |
| | | | | [Oryza sativa Japonica Group] | | | | |
| | | | | &gt:gi\|218199540\|gb\|EEC81967.1\| | | | | |
| | | | | hypothetical protein OsI_25868 | | | | |
| | | | | [Oryza sativa Indica Group] | | | | |
| | | NP_001174604 | 297724480 | Os06g0151300 [Oryza sativa Japonica Group] | 0.923077 | Oryza sativa Japonica Group | 3966 | 5233 |
| | | | | &gt:gi\|222634966\|gb\|EEE65098.1\| | | | | |
| | | | | hypothetical protein OsJ_20146 | | | | |
| | | | | [Oryza sativa Japonica Group] | | | | |
| | | | | &gt:gi\|255676723\|dbj\|BAH93332.1\| | | | | |
| | | | | Os06g0151300 [Oryza sativa Japonica Group] | | | | |
| | | XP_002534307 | 255587545 | synbindin, putative [Ricinus communis] | 0.839161 | Ricinus communis | 3967 | 5234 |
| | | | | &gt:gi\|223525526\|gb\|EEF28075.1\| | | | | |
| | | | | synbindin, putative [Ricinus communis] | | | | |
| | | XP_002281149 | 225450513 | PREDICTED: hypothetical protein | 0.846154 | Vitis vinifera | 3968 | 5235 |
| | | | | [Vitis vinifera] | | | | |
| | | | | &gt:gi\|147807581\|emb\|CAN66318.1\| | | | | |
| | | | | hypothetical protein VITISV_040622 | | | | |
| | | | | [Vitis vinifera] | | | | |
| | | | | &gt:gi\|296089818\|emb\|CBI39637.3\| | | | | |
| | | | | unnamed protein product | | | | |
| | | | | [Vitis vinifera] | | | | |
| | | XP_002314001 | 224105980 | predicted protein [Populus trichocarpa] | 0.839161 | Populus trichocarpa | 3969 | 5236 |
| | | | | &gt:gi\|222850409\|gb\|EEE87956.1\| | | | | |
| | | | | predicted protein [Populus trichocarpa] | | | | |

Target sequences according to the teachings of the invention can be overexpressed or silenced as described herein. Methods of generating transgenic plants are described in Example 6, selection according to expression level is described in Example 7, selection according to tolerance to abiotic stress is described in Examples 8 and 9, above. Generally, target genes of upregulated miRNAs are contemplated to be downregulated; conversely target genes of downregulated miRNAs are contemplated to be upregulated according to the present teachings.

TABLE 23
Abbreviations of Plant Species

| | | Abbrevi- |
|---|---|---|
| Common Name | Organism Name | ation |
| Peanut | Arachis hypogaea | ahy |
| Arabidopsis lyrata | Arabidopsis lyrata | aly |
| Rocky Mountain Columbine | Aquilegia coerulea | aqc |
| Tausch's goatgrass | Aegilops taushii | ata |
| Arabidopsis thaliana | Arabidopsis thaliana | ath |
| Grass | Brachypodium distachyon | bdi |
| Brassica napus canola | Brassica napus | bna |
| (“liftit”) | | |
| Brassica oleracea wild | Brassica oleracea | bol |
| cabbage | | |
| Brassica rapa yellow | Brassica rapa | bra |
| mustard | | |
| Clementine | Citrus clementine | ccl |
| Orange | Citrus sinensis | csi |
| Trifoliate orange | Citrus trifoliata | ctr |
| Glycine max | Glycine max | gma |
| Wild soybean | Glycine soja | gso |
| Barley | Hordeum vulgare | hvu |
| Lotus japonicus | Lotus japonicus | lja |
| Medicago truncatula - | Medicago truncatula | mtr |
| Barrel Clover (“tiltan”) | | |
| Oryza sativa | Oryza sativa | osa |
| European spruce | Picea abies | pab |
| Physcomitrella patens | Physcomitrella patens | ppt |
| (moss) | | |
| Pinus taeda - Loblolly | Pinus taeda | pta |
| Pine | | |
| Populus trichocarpa - | Populus trichocarpa | ptc |
| black cotton wood | | |
| Castor bean (“kikayon”) | Ricinus communis | rco |
| Sorghum bicolor Dura | Sorghum bicolor | sbi |
| tomato microtom | Solanum lycopersicum | sly |
| Selaginella | Selaginella | smo |
| moellendorffii | moellendorffii | |
| Sugarcane | Saccharum spp | ssp |
| Triticum aestivum | Triticum aestivum | tae |
| cacao tree and cocoa tree | Theobroma cacao | tcc |
| Vitis vinifera Grapes | Vitis vinifera | vvi |
| corn | Zea mays | zma |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of improving abiotic stress tolerance, nitrogen use efficiency, biomass, vigor or yield of a plant, the method comprising expressing within the plant an exogenous polynucleotide having a nucleic acid sequence at least 90% identical to SEQ ID NOs: 103, 101-102, 104-216, 223-227, 264-416, wherein said nucleic acid sequence is capable of regulating abiotic stress tolerance of the plant, thereby improving abiotic stress tolerance, nitrogen use efficiency, biomass, vigor or yield of the plant.

2. A transgenic plant exogenously expressing a polynucleotide having a nucleic acid sequence at least 90% identical to SEQ ID NOs: 1-216, 223-227, 264-416, 615-626 or 639, wherein said nucleic acid sequence is capable of regulating abiotic stress tolerance of the plant.

3. The transgenic plant of claim 2, wherein said polynucleotide has a nucleic acid sequence selected from the group consisting of SEQ ID NOs: 1-216, 223-227, 264-416, 615-626 or 639.

4. The method of claim 1, wherein said exogenous polynucleotide encodes a precursor of said nucleic acid sequence.

5. The method of claim 1, wherein said precursor is at least 60% identical to SEQ ID NO: 217-222, 417-421 or 458-614.

6. The method of claim 1, wherein said exogenous polynucleotide encodes a miRNA or a precursor thereof.

7. The method of claim 1, wherein said exogenous polynucleotide encodes a siRNA.

8. The method of claim 1, wherein said exogenous polynucleotide is selected from the group consisting of SEQ ID NO: 103, 101-102, 104-216, 217-222, 223-227, 264-416, 417-421 or 458-614.

9. An isolated polynucleotide having a nucleic acid sequence at least 90% identical to SEQ ID NO: 16-113, 117-216, wherein said nucleic acid sequence is capable of regulating abiotic stress tolerance of a plant.

10. The isolated polynucleotide of claim 9, wherein said nucleic acid sequence is as set forth in SEQ ID NO: 16-113, 117-216.

11. The isolated polynucleotide of claim 9, wherein said polynucleotide encodes a precursor of said nucleic acid sequence.

12. The isolated polynucleotide of claim 9, wherein said polynucleotide encodes a miRNA or a precursor thereof.

13. The isolated polynucleotide of claim 9, wherein said polynucleotide encodes a siRNA.

14. A nucleic acid construct comprising the isolated polynucleotide of claim 9 under the regulation of a cis-acting regulatory element.

15. The nucleic acid construct of claim 14, wherein said cis-acting regulatory element comprises a promoter.

16. The nucleic acid construct of claim 15, wherein said promoter comprises a tissue-specific promoter.

17. The nucleic acid construct of claim 16, wherein said tissue-specific promoter comprises a root specific promoter.

18. A method of improving abiotic stress tolerance, nitrogen use efficiency, biomass, vigor or yield of a plant, the method comprising expressing within the plant an exogenous polynucleotide which downregulates an activity or expression of a gene encoding an RNAi molecule having a nucleic acid sequence at least 90% identical to a nucleic acid sequence selected from the group consisting of SEQ ID NOs: 1-100, 615-626 and 639, thereby improving abiotic stress tolerance, nitrogen use efficiency, biomass, vigor or yield of a plant.

19. A transgenic plant exogenously expressing a polynucleotide which downregulates an activity or expression of a gene encoding an RNAi molecule having a nucleic acid sequence at least 90% identical to a nucleic acid sequence selected from the group consisting of SEQ ID NOs: 1-100, 615-626 and 639.

20. An isolated polynucleotide which downregulates an activity or expression of a gene encoding an RNAi molecule having a nucleic acid sequence selected from the group consisting of SEQ ID NOs: 1-100, 615-626 and 639, 627-638 and 640.

21. The method of claim 18, wherein said polynucleotide encodes a miRNA-Resistant Target as set forth in Tables 14-16.

22. The method of claim 21, wherein said polynucleotide encoding miRNA-Resistant Target is as set forth in SEQ ID NO: 877-886, 893-913, 1226-1535.

23. The method of claim 18, wherein said isolated polynucleotide encodes a target mimic as set forth in Tables 17-19.

24. The method of claim 21, wherein said polynucleotide encoding said target mimic is as set forth in SEQ ID NO:1741-1815.

25. A nucleic acid construct comprising the isolated polynucleotide of claim 20 under the regulation of a cis-acting regulatory element.

26. The nucleic acid construct of claim 25, wherein said cis-acting regulatory element comprises a promoter.

27. The nucleic acid construct of claim 26, wherein said promoter comprises a tissue-specific promoter.

28. The nucleic acid construct of claim 27, wherein said tissue-specific promoter comprises a root specific promoter.

29. The method of claim 1, further comprising growing the plant under abiotic stress.

30. The method of claim 29, wherein said abiotic stress is selected from the group consisting of salinity, drought, water deprivation, flood, etiolation, low temperature, high temperature, heavy metal toxicity, anaerobiosis, nutrient deficiency, nutrient excess, low nitrogen, atmospheric pollution and UV irradiation.

31. The method of claim 1, wherein the plant is a monocotyledon.

32. The method of claim 1, wherein the plant is a dicotyledon.

33. A method of improving abiotic stress tolerance, nitrogen use efficiency, biomass, vigor or yield of a plant, the method comprising expressing within the plant an exogenous polynucleotide encoding a polypeptide having an amino acid sequence at least 80% homologous to SEQ ID NOs: 1861-1869, 1892-1915, 1921-1924, 1931-1939, 1952-1963, 2010-2014, 2327-2355, 2763-3040, 3044-3163, 3175-3269, 3313-3323, 3458-3944 or 3950-3969, wherein said polypeptide is capable of regulating abiotic stress tolerance of the plant, thereby improving abiotic stress tolerance, nitrogen use efficiency, biomass, vigor or yield of the plant.

34. A transgenic plant exogenously expressing a polynucleotide encoding a polypeptide having an amino acid sequence at least 80% homologous to SEQ ID NOs: 1816-2014, 2183-2355, 2500-3969, wherein said polypeptide is capable of regulating nitrogen use efficiency of the plant.

35. A nucleic acid construct comprising a polynucleotide encoding a polypeptide having an amino acid sequence at least 80% homologous to SEQ ID NOs: 1816-2014, 2183-2355, 2500-3969, wherein said polypeptide is capable of regulating abiotic stress tolerance of a plant, and wherein said polynucleotide is under a transcriptional control of a cis-acting regulatory element.

36. The method of claim 33, wherein said polynucleotide is selected from the group consisting of SEQ ID NO: 2053-2061, 2080-2101, 2106-2109, 2111-2116, 2126-2136, 2178-2182, 2478-2499, 4185-4418, 4422-4527, 4539-4624, 4661-4670, 4787-5213 and 5219-5238.

37. The method of claim 33, wherein said polypeptide is selected from the group consisting of SEQ ID NO: 1861-1869, 1892-1915, 1921-1924, 1931-1939, 1952-1963, 2010-2014, 2327-2355, 2763-3040, 3044-3163, 3175-3269, 3313-3323, 3458-3944 and 3950-3969.

38. The nucleic acid construct of claim 35, wherein said cis-acting regulatory element comprises a promoter.

39. The nucleic acid construct of claim 38, wherein said promoter comprises a tissue-specific promoter.

40. The nucleic acid construct of claim 39, wherein said tissue-specific promoter comprises a root specific promoter.

41. The method of claim 33, further comprising growing the plant under water deprivation conditions.

42. The method of claim 33, further comprising growing the plant under salinity stress.

43. The method of claim 33, further comprising growing the plant under high temperature stress.

44. The method of claim 33, further comprising growing the plant under abiotic stress.

45. The method of claim 44, wherein said abiotic stress is selected from the group consisting of salinity, drought, water deprivation, flood, etiolation, low temperature, high temperature, heavy metal toxicity, anaerobiosis, nutrient deficiency, nutrient excess, atmospheric pollution and UV irradiation.

46. The method of claim 33, wherein the plant is a monocotyledon.

47. The method of claim 33, wherein the plant is a dicotyledon.

48. A method of improving abiotic stress tolerance, nitrogen use efficiency, biomass, vigor or yield of a plant, the method comprising expressing within the plant an exogenous polynucleotide which downregulates an activity or expression of a polypeptide having an amino acid sequence at least 80% homologous to SEQ ID NOs: 1816-1860, 1870-1891, 1916-1920, 1925-1930, 1940-1951, 1964-2009, 2183-2326, 2500-2762, 3041-3043, 3164-3174, 3270-3312, 3324-3457, 3945-3949, wherein said polypeptide is capable of regulating abiotic stress tolerance of the plant, thereby improving abiotic stress tolerance, nitrogen use efficiency, biomass, vigor or yield of the plant.

49. A transgenic plant exogenously expressing a polynucleotide which downregulates an activity or expression of a polypeptide having an amino acid sequence at least 80% homologous to SEQ ID NOs: 1816-1860, 1870-1891, 1916-1920, 1925-1930, 1940-1951, 1964-2009, 2183-2326, 2500-2762, 3041-3043, 3164-3174, 3270-3312, 3324-3457, 3945-3979, wherein said polypeptide is capable of regulating abiotic stress tolerance of the plant.

50. A nucleic acid construct comprising a polynucleotide which downregulates an activity or expression of a polypeptide having an amino acid sequence at least 80% homologous to SEQ ID NOs: 1816-1860, 1870-1891, 1916-1920, 1925-1930, 1940-1951, 1964-2009, 2183-2326, 2500-2762, 3041-3043, 3164-3174, 3270-3312, 3324-3457, 3945-3949, wherein said polypeptide is capable of regulating abiotic stress tolerance of a plant, said nucleic acid sequence being under the regulation of a cis-acting regulatory element.

51. The method of claim 48, wherein said polynucleotide acts by a mechanism selected from the group consisting of sense suppression, antisense suppression, ribozyme inhibition, gene disruption.

52. The nucleic acid construct of claim 50, wherein said cis-acting regulatory element comprises a promoter.

53. The nucleic acid construct of claim 52, wherein said promoter comprises a tissue-specific promoter.

54. The nucleic acid construct of claim 53, wherein said tissue-specific promoter comprises a root specific promoter.

55. The method of claim 48, further comprising growing the plant under water deprivation conditions.

56. The method of claim 48, further comprising growing the plant under salinity stress.

57. The method of claim 48, further comprising growing the plant under high temperature stress.

58. The method of claim 48, further comprising growing the plant under abiotic stress.

59. The method of claim 58, wherein said abiotic stress is selected from the group consisting of salinity, drought, water deprivation, flood, etiolation, low temperature, high temperature, heavy metal toxicity, anaerobiosis, nutrient deficiency, nutrient excess, atmospheric pollution and UV irradiation.
